(19) United States
(12) Patent Application Publication
(10) Pub. No.: US 2014/0309192 A1
(43) Pub. Date: Oct. 16, 2014

(54) MODULATORS OF THE G PROTEIN-COUPLED MAS RECEPTOR AND THE TREATMENT OF DISORDERS RELATED THERETO

(71) Applicant: ARENA PHARMACEUTICALS, INC., San Diego, CA (US)

(72) Inventors: Thuy-Anh Tran, San Diego, CA (US); Anthony C. Blackburn, San Diego, CA (US); Bryan A. Kramer, San Diego, CA (US); Maiko Nagura, La Jolla, CA (US); Carleton R. Sage, Encinitas, CA (US); Young-Jun Shin, San Diego, CA (US); Ning Zou, San Diego, CA (US)

(21) Appl. No.: 14/356,792

(22) PCT Filed: Nov. 7, 2012
(86) PCT No.: PCT/US2012/063793

§ 371 Date: May 7, 2014

Related U.S. Application Data
(60) Provisional application No. 61/557,088, filed on Nov. 8, 2011.
(60) Provisional application No. 61/718,290, filed on Oct. 25, 2012.

(51) Int. Cl. C07D 295/135; C07F 9/6509; C07D 205/04; C07D 213/82; C07D 241/24; C07D 335/02; C07D 265/30; C07D 241/08

(52) U.S. Cl. CPC C07D 295/135; C07D 265/30; C07F 9/650958; C07D 241/08; C07D 213/82; C07D 241/24; C07D 335/02; C07D 205/04
USPC 514/85; 514/235.8; 514/252.11; 514/255.03; 544/121; 544/337; 544/357; 544/393; 544/365; 514/253.12; 544/374; 514/252.13; 544/372; 514/254.01; 544/359; 514/210.18

(57) ABSTRACT

The present invention relates to compounds of Formula (I) and pharmaceutically acceptable salts, solvates, and hydrates thereof:

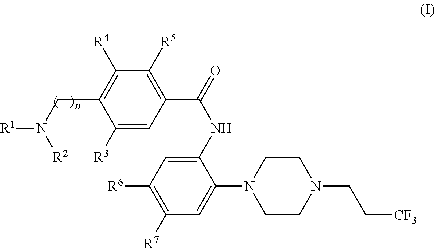

that are useful in methods of treatment and alleviation of diseases and disorders of the heart, brain, kidney, immune, and reproductive system resulting from ischemia, or reperfusion subsequent to ischemia, and any downstream complication(s) related thereto. The present invention further relates to methods of treatment and alleviation of diseases and disorders of the vasculature resulting from vasoconstriction or hypertension and any downstream complication(s) resulting from elevated blood pressure and/or reduced tissue perfusion.

### FIELD OF THE INVENTION

The present invention relates to compounds of Formula (I) and pharmaceutically acceptable salts, solvates, and hydrates thereof that modulate the activity of the Mas receptor, and are useful in methods of treatment and alleviation of diseases and disorders of the heart, brain, kidney, immune, and reproductive system resulting from ischemia, or reperfusion subsequent to ischemia, and any downstream complication(s) related thereto. The present invention further relates to methods of treatment and alleviation of diseases and disorders of the vasculature resulting from vasoconstriction or hypertension and any downstream complication(s) resulting from elevated blood pressure and/or reduced tissue perfusion. Theses diseases and disorders include, for example, vascular disorders, such as: coronary heart disease, atherosclerosis, ischemia, reperfusion injury, angina pectoris, myocardial infarction, the no-reflow phenomenon, hypertension, anxiety, transient ischemic attack, erectile dysfunction, ischemic colitis, mesenteric ischemia, acute limb ischemia, skin discoloration caused by reduced blood flow to the skin, renal artery stenosis, renovascular hypertension, renal failure, chronic kidney disease, and diabetic nephropathy; and calcium-signaling disorders such as: arrhythmia, tachycardia, bradycardia, supraventricular arrhythmia, atrial fibrillation, atrial flutter, paroxysmal supraventricular tachycardia, Wolff-Parkinson-White syndrome, ventricular arrhythmia, ventricular tachycardia, ventricular fibrillation, reperfusion arrhythmia, and reperfusion-induced cardiomyocyte cell death.

### BACKGROUND OF THE INVENTION

G protein-coupled receptors (GPCRs) share the common structural motif of having seven sequences of twenty-two to twenty-four hydrophobic amino acids that form seven alpha helices, each of which spans the cell membrane. The transmembrane helices are joined by strands of amino acids including a longer strand between the fourth and fifth transmembrane helices on the extracellular side of the membrane. Another longer strand, composed primarily of hydrophilic amino acids, joins transmembrane helices five and six on the intracellular side of the membrane. The carboxy terminus of the receptor lies intracellularly with the amino terminus residing in the extracellular space. It is believed that the intracellular amino acid strand joining helices five and six, as well as the carboxy terminus, interact with the G protein. In general, the G proteins that have been identified include G_{q}, Gₛ, Gᵢ, and Gₒ.

Under physiological conditions, GPCRs exist in the cell membrane in equilibrium between two different states or conformations: an “inactive” state and an “active” state. A receptor in an inactive state is unable to link to the intracellular transduction pathway to produce a biological response. A change of the receptor conformation to the active state allows linkage to the transduction pathway and produces a biological response. Physiologically, these conformational changes are induced in response to binding of a molecule to the receptor. Several types of biological molecules can bind to specific receptors, such as peptides, hormones or lipids, and can cause a cellular response. Modulation of particular cellular responses can be extremely useful for the treatment of disease states, as can a number of chemical agents that act on GPCRs.

The Mas receptor (Mas, or alternatively Mas1) is a class I rhodopsin-like GPCR. In mammals, Mas is expressed predominantly in brain and testis with moderate levels of expression in heart and kidney, and lower expression in several other tissues (Alenina N., et al., Exp Physiol 93:528-537 (2008); Metzger R., et al., FEBS Lett 357:27-32 (1995); Villar A. J. and Pedersen R. A., Nat Genet. 8:373-379 (1994); Young D., et al., Cell 45:711-719 (1986)). The Mas proto-oncogene encodes a GPCR protein (Mas) and was first detected in vivo by its tumorigenic properties which originate from rearrangement of its 5′ flanking region (Young, D., et al., Cell 45:711-719 (1996)). Subsequent studies have indicated that the tumorigenic properties of Mas appear to be negligible.

Although it was suggested in early studies that Mas is an angiotensin II (Ang II) receptor (Jackson T. R., et al. Nature 335: 437-440, (1988)), later studies demonstrated that Ang II-mediated intracellular signaling in Mas-transfected cells was only observed in cells endogenously expressing the AT₁ receptor (Ambroz C., et al. Biochim Biophys Acta 1133: 107-111, (1991)). Dong et al. reported that the Mas receptor did not bind to angiotensins I and II, but did bind to a peptide called NPFF, although fairly weakly (EC₅₀ about 400 nM) (Dong, et al., Cell 106:619-632 (2001)). Currently there is still uncertainty about the endogenous ligand for the Mas receptor although the peptide angiotensin-(1-7) (Ang-(1-7)), which is derived from Ang II, has recently been described as the endogenous agonist of Mas (Santos R. A., et al. Proc Natl Acad Sci USA 100: 8258-8263, 2003).

The present invention describes, inter alia, the proximal signaling pathways activated by Mas and the role of the Mas receptor in cardiac ischemia/reperfusion (I/R) injury in vivo. In addition, described herein include small molecule, non-peptide modulators of the Mas-G_{q}-phospholipase C(PLC) signaling pathway. These results demonstrate that the Mas receptor is a G_{q}-coupled receptor and that a reduction of Mas signaling activity, either by genetic alteration or with the pharmacological use of Mas inverse agonists, is cardioprotective during ischemia/reperfusion injury. These results further indicate that therapies aimed at reducing Mas receptor G_{q}-PLC signaling, such as the use of inverse agonists and particularly those inverse agonists described herein, represent a promising new strategy for treatment of ischemia/reperfusion injury, such as in organs where the Mas receptor is expressed, for example, heart, kidney, brain, and testis.

Citation of any reference throughout this application is not to be construed as an admission that such reference is prior art to the present application.

### SUMMARY OF THE INVENTION

The present invention relates to, inter alia, certain amide derivatives and pharmaceutically acceptable salts, solvates, and hydrates thereof, which are useful, for example, in methods of treatment or alleviation of ischemia-related disorders of the heart, brain, kidney, and reproductive systems.

While the literature cited above may indicate that an agonist of the Mas receptor would be cardioprotective and decrease blood pressure, applicants have unexpectedly identified compounds that can act as inverse agonists of the Mas receptor which are cardioprotective and do not raise blood pressure.

One aspect of the present invention is directed to compounds, as described herein, and pharmaceutically acceptable salts, solvates, and hydrates thereof, which bind to and modulate the activity of a GPCR, referred to herein as Mas, and uses related thereto.

One aspect of the present invention is directed to compounds of Formula (I) and pharmaceutically acceptable salts, solvates, and hydrates thereof:

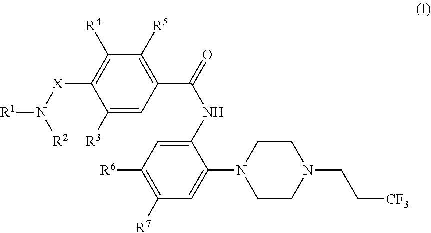

wherein:

X is CH₂ or CH₂CH₂; or X is absent;

R⁴, R⁵, R⁶, and R⁷ are each selected independently from: H and halogen; and

(A) R¹ is selected from: H, C₁-C₆ alkyl, C₁-C₆-alkyl-O—C₁-C₆-alkyl, C₃-C₇ cycloalkyl, C₄-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy;

R² is selected from: H and C₁-C₆ alkyl, wherein said C₁-C₆ alkyl is optionally substituted with one or more substituents selected from: hydroxyl and cyano; and

R³ is selected from: H and halogen; or

(B) R¹ and R² together with the nitrogen atom to which they are both bonded form a group selected from: heteroaryl and heterocyclyl, each optionally substituted with one or more substituents selected from: C₁-C₆ alkoxycarbonyl, C₁-C₆ alkoxycarbonylamino, C₁-C₆ alkyl, C₁-C₆ alkylcarboxamide, C₁-C₆ alkylsulfonyl, amino, C₃-C₇ cycloalkyl, C₄-C₁₃ cycloalkylalkyl, carboxamide, carboxyl, C₂-C₆ dialkylamino, C₂-C₆ dialkylcarboxamide, heteroaryl-C₁-C₆-alkyl, heterocyclyl, heterocyclyl-C₁-C₆-alkyl, hydroxyl, hydroxyheterocyclyl, and oxo, wherein said C₁-C₆ alkyl and C₁-C₆ alkylcarboxamide are each optionally substituted with one or more substituents selected from: carboxyl, hydroxyl, and oxo; and

R³ is selected from: H and halogen; or

(C) R¹ is selected from: H, C₁-C₆ alkyl, C₁-C₆-alkyl-O—C₁-C₆-alkyl, C₃-C₇ cycloalkyl, C₄-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy; and

R² and R³ together form CH₂.

One aspect of the present invention pertains to the following compound and pharmaceutically acceptable salts, solvates, hydrates, and crystalline forms thereof: (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide.

One aspect of the present invention pertains to compositions comprising a compound of the present invention or a crystalline form of the present invention.

One aspect of the present invention pertains to pharmaceutical products selected from: a pharmaceutical composition, a formulation, a unit dosage form, and a kit, each comprising a compound of the present invention or a crystalline form of the present invention.

One aspect of the present invention pertains to pharmaceutical compositions comprising a compound of the present invention or a crystalline form of the present invention, and a pharmaceutically acceptable carrier.

One aspect of the present invention pertains to methods for preparing a pharmaceutical composition comprising the step of admixing a compound of the present invention or a crystalline form of the present invention, and a pharmaceutically acceptable carrier.

One aspect of the present invention pertains to compositions comprising a compound of the present invention or a crystalline form of the present invention, and a second pharmaceutical agent.

One aspect of the present invention pertains to methods for preparing a composition comprising the step of admixing a compound of the present invention or a crystalline form of the present invention, and a second pharmaceutical agent.

One aspect of the present invention pertains to pharmaceutical products selected from: a pharmaceutical composition, a formulation, a dosage form, a combined preparation, a twin pack, and a kit; comprising a compound of the present invention or a crystalline form of the present invention and a second pharmaceutical agent.

One aspect of the present invention pertains to pharmaceutical compositions comprising a compound of the present invention or a crystalline form of the present invention, a second pharmaceutical agent, and a pharmaceutically acceptable carrier.

One aspect of the present invention pertains to methods for preparing a pharmaceutical composition comprising the step of admixing a compound of the present invention or a crystalline form of the present invention, a second pharmaceutical agent, and a pharmaceutically acceptable carrier.

One aspect of the present invention pertains to methods for the treatment of a Mas receptor-mediated disorder in an individual, comprising administering to the individual in need thereof, a therapeutically effective amount of a compound of the present invention; a crystalline form of the present invention; a composition of the present invention; a pharmaceutical product of the present invention; or a pharmaceutical composition of the present invention.

One aspect of the present invention pertains to methods for the treatment of a Mas receptor-mediated disorder in an individual, comprising prescribing to the individual in need thereof, a therapeutically effective amount of a compound of the present invention; a crystalline form of the present invention; a composition of the present invention; a pharmaceutical product of the present invention; or a pharmaceutical composition of the present invention.

One aspect of the present invention pertains to the use of a compound of the present invention; a crystalline form of the present invention; or a composition of the present invention; in the manufacture of a medicament for the treatment of a Mas receptor-mediated disorder.

One aspect of the present invention pertains to a compound of the present invention; a crystalline form of the present invention; a composition of the present invention; a pharmaceutical product of the present invention; or a pharmaceutical composition of the present invention; for use in a method of treatment of the human or animal body by therapy.

One aspect of the present invention pertains to a compound of the present invention; a crystalline form of the present invention; a composition of the present invention; a pharmaceutical product of the present invention; or a pharmaceutical composition of the present invention; for use in a method of treatment of a Mas receptor-mediated disorder.

One aspect of the present invention pertains to methods for the treatment of a Mas receptor-mediated disorder in an individual, comprising administering to the individual in need thereof, a therapeutically effective amount of a compound of the present invention; a crystalline form of the present invention; a composition of the present invention; a pharmaceutical product of the present invention; or a pharmaceutical composition of the present invention; each in combination with a therapeutically effective amount of a second pharmaceutical agent.

One aspect of the present invention pertains to methods for the treatment of a Mas receptor-mediated disorder in an individual, comprising prescribing to the individual in need thereof, a therapeutically effective amount of a compound of the present invention; a crystalline form of the present invention; a composition of the present invention; a pharmaceutical product of the present invention; or a pharmaceutical composition of the present invention; each in combination with a therapeutically effective amount of a second pharmaceutical agent.

One aspect of the present invention pertains to the use of a compound of the present invention; a crystalline form of the present invention; or a composition of the present invention; each in combination with a second pharmaceutical agent in the manufacture of a medicament for the treatment of a Mas receptor-mediated disorder.

One aspect of the present invention pertains to the use of a pharmaceutical agent in combination with a compound of the present invention; a crystalline form of the present invention; or a composition of the present invention; in the manufacture of a medicament for the treatment of a Mas receptor-mediated disorder.

One aspect of the present invention pertains to a compound of the present invention; a crystalline form of the present invention; a composition of the present invention; a pharmaceutical product of the present invention; or a pharmaceutical composition of the present invention; each in combination with a second pharmaceutical agent for use in a method of treatment of the human or animal body by therapy.

One aspect of the present invention pertains to a compound of the present invention; a crystalline form of the present invention; a composition of the present invention; a pharmaceutical product of the present invention; or a pharmaceutical composition of the present invention; each in combination with a second pharmaceutical agent for use in a method of treatment of a Mas receptor-mediated disorder.

One aspect of the present invention pertains to a pharmaceutical agent in combination with a compound of the present invention; a crystalline form of the present invention; a composition of the present invention; a pharmaceutical product of the present invention; or a pharmaceutical composition of the present invention; for use in a method of treatment of the human or animal body by therapy.

One aspect of the present invention pertains to a pharmaceutical agent in combination with a compound of the present invention; a crystalline form of the present invention; a composition of the present invention; a pharmaceutical product of the present invention; or a pharmaceutical composition of the present invention; for use in a method of treatment of a Mas receptor-mediated disorder.

One aspect of the present invention pertains to a pharmaceutical product selected from: a pharmaceutical composition, a formulation, a unit dosage form, and a kit; each comprising a compound of the present invention; a crystalline form of the present invention; or a composition of the present invention; in combination with a second pharmaceutical agent; for use in a method of treatment of the human or animal body by therapy.

One aspect of the present invention pertains to a pharmaceutical product selected from: a pharmaceutical composition, a formulation, a unit dosage form, and a kit; each comprising a compound of the present invention; a crystalline form of the present invention; or a composition of the present invention; in combination with a second pharmaceutical agent; for use in a method of treatment of a Mas receptor-mediated disorder.

One aspect of the present invention pertains to a composition of the present invention; methods of the present invention; a pharmaceutical product of the present invention; a pharmaceutical composition of the present invention; a use of the present invention; a compound of the present invention; a crystalline form of the present invention; or a pharmaceutical agent of the present invention; wherein the pharmaceutical agent or the second pharmaceutical agent is selected from: an ACE inhibitor, a beta blocker, a calcium channel blocker, a diuretic, a nitrate, a statin, aspirin, an anti-platelet, adenosine, an endothelin receptor antagonist, and a PDE5 inhibitor.

These and other aspects of the invention disclosed herein will be set forth in greater detail as the patent disclosure proceeds.

### BRIEF DESCRIPTION OF THE DRAWINGS

FIG. 1 shows a general synthetic scheme for the preparation of compounds of Formula (I) wherein X is absent.

FIG. 2 shows a general synthetic scheme for the preparation of useful intermediates for preparing compounds of Formula (I) wherein X is CH₂.

FIG. 3 shows a general synthetic scheme for the preparation of compounds of Formula (I) wherein X is CH₂.

FIG. 4A shows a general synthetic scheme for the preparation of certain compounds of Formula (I) wherein X is CH₂ or CH₂CH₂, and R² and R³ together form CH₂. In certain embodiments, R² and R³ together with the nitrogen atom to which R² is bonded, and the phenyl ring to which R³ is bonded, and X form a group selected from: 1,2,3,4-tetrahydroisoquinolinyl and isoindolinyl.

FIG. 4B shows a general synthetic scheme for the preparation of certain compounds of Formula (I) using amines and protected amines with bis(2,5-dioxopyrrolidin-1-yl) carbonate to form ureas.

FIG. 4C shows a general synthetic scheme for the preparation of certain compounds of Formula (I). Using (2,5-dioxopyrrolidin-1-yloxy)carbonylamino intermediates and protected amines, such as BOC protected amines, certain cyclic ureas of the present invention can be prepared, see Example 1.439.

FIG. 5 shows the dose dependent effect of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) on reduction of myocardial infarction size in rats subjected to coronary artery ligation and reperfusion.

FIG. 6 shows the effect of Compound 170 on mean arterial pressure (MAP) measured at drug steady state (25 min after starting dosing).

FIG. 7 shows a powder X-ray diffraction (PXRD) pattern for a sample containing Compound 170 as prepared according to Example 2.5A.

FIG. 8 shows a differential scanning calorimetry (DSC) thermogram for a sample containing Compound 170 as prepared according to Example 2.5A.

FIG. 9 shows a powder X-ray diffraction (PXRD) pattern for a sample containing Compound 170 as prepared according to Example 2.5B.

FIG. 10 shows a differential scanning calorimetry (DSC) thermogram for a sample containing Compound 170 as prepared according to Example 2.5B.

FIG. 11 shows a dynamic moisture sorption (DMS) analysis for a sample containing Compound 170 as prepared according to Example 2.5B.

FIG. 12 shows a powder X-ray diffraction (PXRD) pattern for a sample containing Compound 170 dihydrochloride.

FIG. 13 shows a thermogravimetric analysis (TGA) thermogram of a sample containing Compound 170 dihydrochloride.

FIG. 14 shows a dynamic moisture sorption (DMS) analysis of a sample containing Compound 170 dihydrochloride.

FIG. 15 shows a powder X-ray diffraction (PXRD) pattern for a sample containing Compound 170 dihydrochloride hydrate.

FIG. 16 shows a thermogravimetric analysis (TGA) thermogram of a sample containing Compound 170 dihydrochloride hydrate.

FIG. 17 shows a dynamic moisture sorption (DMS) analysis of a sample containing Compound 170 dihydrochloride hydrate.

FIG. 18 shows a powder X-ray diffraction (PXRD) pattern for a sample containing Compound 170 dihydrochloride solvate as prepared according to Example 2.8.

FIG. 19 shows a thermogravimetric analysis (TGA) thermogram of a sample containing Compound 170 dihydrochloride solvate as prepared according to Example 2.8.

FIG. 20 shows a dynamic moisture sorption (DMS) analysis of a sample containing Compound 170 dihydrochloride solvate as prepared according to Example 2.8.

FIG. 21 shows a powder X-ray diffraction (PXRD) pattern for a sample containing Compound 170 sulfate solvate as prepared according to Example 2.9.

FIG. 22 shows a thermogravimetric analysis (TGA) thermogram of a sample containing Compound 170 sulfate solvate as prepared according to Example 2.9.

FIG. 23 shows a powder X-ray diffraction (PXRD) pattern for a sample containing Compound 170 di-mesylate.

FIG. 24 shows a thermogravimetric analysis (TGA) thermogram of a sample containing Compound 170 di-mesylate.

FIG. 25 shows a dynamic moisture sorption (DMS) analysis of a sample containing Compound 170 di-mesylate. The isotherm adsorption and desorption cycles show a plateau forming between 50 and 86% relative humidity (RH). This is consistent with a hydrate as the weight gain matches the theoretical amount of water, 2.33% for a monohydrate. The critical water activity for this hydrate is between 0.3 and 0.7. The hydrate is lost at lower RH to give Compound 170 di-mesylate.

FIG. 26 shows Mas mRNA expression analysis by RT-PCR in rat heart. cDNA from adult male Sprague-Dawley rat atria, right ventricle and left ventricle was tested for Mas receptor mRNA expression. GAPDH expression in the same samples was used as an internal control for cDNA quality. Results are representative of three independent experiments.

FIG. 27 shows cellular expression of Mas in coronary arteries. Adult rat ventricular cryosections were co-stained with antibodies for Mas and SM-actin (a marker for smooth muscle cells) or Mas and vWF (a marker for endothelial cells). Mas protein expression overlapped with markers for smooth muscle cells and endothelial cells indicating expression in both smooth muscle and endothelial cells in coronary arteries.

FIG. 28 shows Mas mRNA expression analysis by RT-PCR in human cardiovascular cDNA panel. cDNA prepared from human cardiovascular and non-cardiovascular (placenta) tissues was analyzed for Mas mRNA expression. Actin mRNA expression was measured and used as a control for cDNA quality. Results are representative of three independent experiments.

FIG. 29 shows immunohistochemical staining of Mas in human left ventricular sections. Human myocardial cryosections were stained with either Mas antibody or Mas antibody preabsorbed with blocking peptide. Panel A shows positive staining for Mas in cardiomyocytes. Panel B shows positive staining for Mas in coronary arteries (black arrow). Preincubation of Mas antibody with the blocking peptide demonstrates the level of non-specific staining in human myocardial sections. The dark staining indicates hematoxylin counterstaining of cell nuclei.

FIG. 30 shows constitutive Mas G_{q} activity for human and rat receptor constructs. Human and rat Mas receptors were transiently transfected into HEK293 cells and G_{q} signaling was measured using an HTRF IP1 assay performed 48 h post transfection. HEK293 cells transfected with pHM6 empty vector (vector) served as a control. n=14 per group; ***p<0.001 vs vector control.

FIG. 31 shows a Mas agonist and an inverse agonist that modulate G_{q} in HEK293 cells expressing human Mas. Increasing concentrations of Mas agonist (AR234960) and inverse agonist (AR244555) (Zhang, T., et. al., Am J Physiol Heart Circ Physiol 302:H299-H311, (2012)) were incubated with HEK293 cells stably expressing human Mas for 4 h, then G_{q} signaling was measured using an HTRF IP1 assay. Measurements were made in triplicate.

FIG. 32 shows a Mas agonist and an inverse agonist that modulate G_{q} in HEK293 cells expressing rat Mas. Mas agonist (AR234960) and inverse agonist (AR244555) were incubated with HEK293 cells stably expressing rat Mas for 4 h, then G_{q} signaling was measured using HTRF IP1 assays. Measurements were made in triplicate.

FIG. 33 shows the effect of the Mas agonist (AR234960) on intracellular Ca²⁺ Levels. Fluorometric assays were used to monitor Ca²⁺ levels in HEK293 cells stably expressing human Mas receptor. Changes in intracellular Ca²⁺ were monitored before and after addition of the Mas agonist AR234960 at indicated concentrations. Measurements were made in triplicate.

FIG. 34 shows Mas agonist-mediated decrease in coronary flow is Mas receptor dependent. Coronary flow was measured in isolated perfused mouse hearts. Mas agonist AR234960 (1 μM) decreased coronary flow significantly in wild type (Mas^{+/+}) mice but not in hearts from Mas knockout (Mas^{-/-}) mice. n=4-6 mice per group; *p<0.05 vs. Mas^{+/+}/vehicle.

FIG. 35 shows the effects of Mas compounds on rat coronary flow. Coronary flow was measured in isolated perfused rat hearts. Coronary flow was significantly increased upon stimulation with Mas inverse agonist AR244555 (5 μM) and significantly decreased with Mas agonist AR234960 (1 μM). Pretreatment with the Mas inverse agonist AR244555 (5 μM) or the PLC inhibitor U-73122 (0.5 μM) prevented the decrease in coronary flow induced by AR234960. Changes in coronary flow induced by AR234960 with the pretreatment of AR244555 or U-73122 were calculated as percentage of the coronary flow at 10 min after AR234960 treatment relative to the coronary flow measured immediately prior to addition of AR234960. In endothelium denuded hearts (Endo (−)), the AR234960-mediated decrease in coronary flow was preserved. n=4-6 hearts per group; ***p<0.001 vs. vehicle.

FIG. 36 shows the effects of Mas compounds on rat coronary flow after ischemia and reperfusion. Coronary flow was measured continuously in isolated perfused rat hearts that were subjected to 30 min of global ischemia followed by 30 min of reperfusion. Mas agonist (1 μM AR234960), Mas inverse agonist (5 μM AR244555) or vehicle (0.01% DMSO) was added to the perfusate during reperfusion. Changes in coronary flow were analyzed at 10 min intervals and are represented as a percentage of baseline flow determined 10 min prior to ischemia. n=6-7 rats per group; *p<0.05 vs. vehicle.

FIG. 37 shows ablation of Mas receptor in mice is cardioprotective during ischemia/reperfusion injury (I/R injury). Regional ischemia/reperfusion injury was produced in mice by ligation of the left anterior descending coronary artery for 30 min followed by release of the ligation (reperfusion). After 2 hr of reperfusion, hearts were removed and infarct size was measured as a percentage of the area at risk (AAR), n=7-9 mice per group; **p<0.01 vs. WT.

FIG. 38 shows the inverse agonist of the Mas receptor (AR244555) was cardioprotective in rats when administered prior to ischemia or immediately before reperfusion. Regional ischemia/reperfusion injury was produced in rats by ligation of the left anterior descending coronary artery for 30 min followed by reperfusion for 2 h. Vehicle (20% HPBCD, i.v.) or the Mas inverse agonist (AR244555, 10 mg/kg i.v.) was administered 10 min before ligation (pre-ischemia) or 3 min before reperfusion (pre-reperfusion). Infarct size was measured as a percentage of the area at risk (AAR); n=8 rats per group; ***p<0.001 vs. vehicle.

FIG. 39 shows expression of Mas receptor following lipopolysaccharide (LPS) stimulation.

FIG. 40 shows expression of mTNFα following LPS stimulation in mice.

FIG. 41 shows suppression of TNFα induction with an inverse agonist of the Mas receptor (Compound 170) in mice.

FIG. 42 shows Mas receptor inverse agonist suppresses paw swelling in the Carrageenan-Induced Inflammatory Paw Swelling Model.

FIG. 43 shows the protocol for the left kidney artery ischemia reperfusion injury model used in Example 9.

FIG. 44 shows Compound 170 improves kidney function compared to vehicle treatment as measured by blood creatinine.

FIG. 45 shows Compound 170 improves kidney function compared to vehicle treatment as measured by blood urea nitrogen (BUN).

FIG. 46 shows the protocol for the transient cerebral ischemia/stroke rat model used in Example 10.

FIG. 47 shows Compound 170 reduced brain damage associated with transient ischemic injury in the rat.

### DETAILED DESCRIPTION OF THE INVENTION

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. Accordingly, all combinations of uses and medical indications described herein specifically embraced by the present invention just as if each and every subcombination of uses and medical indications was individually and explicitly recited herein.

### DEFINITIONS

For clarity and consistency, the following definitions will be used throughout this patent document.

The term “agonist” as used herein refers to a moiety that interacts with and activates a G-protein-coupled receptor, and can thereby initiate a physiological or pharmacological response characteristic of that receptor. For example, an agonist may activate an intracellular response upon binding to a receptor, or enhance GTP binding to a membrane.

The term “antagonist” as used herein refers to a moiety that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

The term “composition” refers to a compound of the present invention, including but not limited to, salts, solvates, and hydrates of a compound of the present invention, in combination with at least one additional component.

The term “Mas” as used herein includes the human amino acid sequences found in GeneBank accession number CR542261, and naturally-occurring allelic variants thereof, and mammalian orthologs thereof. A preferred human Mas for use in screening and testing of the compounds of the invention is provided by the nucleotide sequence and the corresponding amino acid sequence found in GeneBank accession number CR542261.

The term “in need of treatment” and the term “in need thereof” when referring to treatment are used interchangeably and refer to a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” refers to any animal, including mammals, preferably mice, rats, other rodents, rabbits, dogs, cats, swine, cattle, sheep, horses, or primates, and most preferably humans.

The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist, or decreases GTP binding to a membrane. Preferably, the baseline intracellular response is inhibited in the presence of the inverse agonist by at least 30%, more preferably by at least 50% and most preferably by at least 75%, as compared with the baseline response in the absence of the inverse agonist.

The term “modulate or modulating” refers to an increase or decrease in the amount, quality, response or effect of a particular activity, function or molecule.

The term “partial agonist” refers to a moiety that by virtue of binding to a GPCR activates the GPCR so as to elicit an intracellular response mediated by the GPCR, albeit to a lesser extent or degree than does a full agonist.

The term “pharmaceutical composition” refers to a composition comprising at least one active ingredient, such as a compound of Formula (I) or a salt, solvate, or hydrate thereof, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “therapeutically effective amount” refers to the amount of active compound or pharmaceutical agent that elicits the biological or medicinal response in a tissue, system, animal, individual or human that is being sought by a researcher, veterinarian, medical doctor or other clinician or caregiver or by an individual, which includes one or more of the following:

(1) preventing the disease, for example, preventing a disease, condition or disorder in an individual that may be predisposed to the disease, condition or disorder but does not yet experience or display the pathology or symptomatology of the disease;

(2) inhibiting the disease, for example, inhibiting a disease, condition or disorder in an individual that is experiencing or displaying the pathology or symptomatology of the disease, condition or disorder (i.e., arresting further development of the pathology and/or symptomatology); and

(3) ameliorating the disease, for example, ameliorating a disease, condition or disorder in an individual that is experiencing or displaying the pathology or symptomatology of the disease, condition or disorder (i.e., reversing the pathology and/or symptomatology).

### Chemical Group, Moiety or Radical

The term “acetamido” refers to the radical of the formula: —NHC(═O)CH₃.

The term “C₁-C₆ alkoxy” refers to a radical consisting of a C₁-C₆ alkyl group attached to an oxygen atom, wherein C₁-C₆ alkyl has the same definition as found herein. Some embodiments contain 1 to 5 carbons. Some embodiments contain 1 to 4 carbons. Some embodiments contain 1 to 3 carbons. Some embodiments contain 1 or 2 carbons. Examples include, but are not limited to methoxy, ethoxy, n-propoxy, isopropoxy, n-butoxy, tert-butoxy, isobutoxy, and sec-butoxy.

The term “amino” refers to the radical —NH₂.

The term “C₁-C₆ alkoxycarbonyl” refers to a radical consisting of a C₁-C₆ alkoxy group attached to a carbonyl group, wherein C₁-C₆ alkoxy has the same definition as found herein. Examples include, but are not limited to, methoxycarbonyl, ethoxycarbonyl, propoxycarbonyl, isopropoxycarbonyl, n-butoxycarbonyl, isobutoxycarbonyl, and tert-butoxycarbonyl.

The term “C₁-C₆ alkoxycarbonylamino” refers to a radical consisting of a C₁-C₆ alkoxycarbonyl radical attached to an amino group, wherein C₁-C₆ alkoxycarbonyl has the same definition as found herein. Examples include, but are not limited to, ethoxycarbonylamino, isopropoxycarbonylamino, and tert-butoxycarbonylamino.

The term “C₁-C₆ alkyl” refers to a radical consisting of a straight or branched carbon radical consisting of 1 to 6 carbons. Some embodiments contain 2 to 6 carbons. Some embodiments contain 1 to 5 carbons. Some embodiments contain 1 to 4 carbons. Some embodiments contain 1 to 3 carbons. Some embodiments contain 1 or 2 carbons. Examples of an alkyl group include, but are not limited to, methyl, ethyl, n-propyl, isopropyl, n-butyl, sec-butyl, isobutyl, tert-butyl, pentyl, isopentyl, t-pentyl, neopentyl, 1-methylbutyl [i.e., —CH(CH₃)CH₂CH₂CH₃], 2-methylbutyl [i.e., —CH₂CH(CH₃)CH₂CH₃], and n-hexyl.

The term “C₁-C₆ alkyl-O—C₁-C₆ alkyl” refers to a radical consisting of a C₁-C₆ alkyl group attached to an oxygen atom wherein the oxygen is further attached to a C₁-C₆ alkyl group, wherein C₁-C₆ alkyl refers to a straight or branched carbon radical containing 1 to 6 carbons. Examples include, but are not limited to, 2-methoxyethyl (i.e., CH₃—O—CH₂CH₂—), (2-propoxyethyl (i.e., CH₃CH₂CH₂—O—CH₂CH₂—), 2-ethoxyethyl (i.e., CH₃CH₂—O—CH₂CH₂—), and 2-isopropoxyethyl (i.e., (CH₃)₂CH—O—CH₂CH₂—).

The term “C₁-C₆ alkylcarboxamide” refers to a radical consisting of one C₁-C₆ alkyl group attached to either the carbon or the nitrogen of an amide group, wherein C₁-C₆ alkyl has the same definition as found herein. The C₁-C₆ alkylcarboxamide group can be represented by the following formulae:

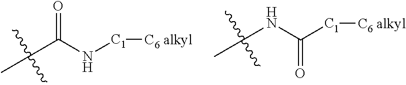

Examples include, but are not limited to, N-methylcarboxamide, N-ethylcarboxamide, N-n-propylcarboxamide, N-isopropylcarboxamide, N-n-butylcarboxamide, N-sec-butylcarboxamide, N-isobutylcarboxamide, and N-tert-butylcarboxamide.

The term “C₁-C₆ alkylsulfinyl” refers to a radical consisting of a C₁-C₆ alkyl group attached to the sulfur of an sulfinyl group (i.e., —S(O)—), wherein C₁-C₆ alkyl has the same definition as described herein. Examples include, but are not limited to, methylsulfinyl, ethylsulfinyl, n-propylsulfinyl, isopropylsulfinyl, n-butylsulfinyl, sec-butylsulfinyl, isobutylsulfinyl, and tert-butylsulfinyl.

The term “C₁-C₆ alkylsulfonyl” refers to a radical consisting of a C₁-C₆ alkyl group attached to the sulfur of a sulfonyl group (i.e., —S(O)₂—), wherein C₁-C₆ alkyl has the same definition as described herein. Examples include, but are not limited to, methylsulfonyl, ethylsulfonyl, n-propylsulfonyl, isopropylsulfonyl, n-butylsulfonyl, sec-butylsulfonyl, isobutylsulfonyl, and tert-butylsulfonyl.

The term “amino-C₁-C₆ alkoxy” refers to a radical consisting of an amino group attached to a carbon atom of a C₁-C₆ alkoxy group, wherein C₁-C₆ alkoxy has the same definition as described herein. Examples include, but are not limited to, 2-aminoethoxy (i.e., H₂NCH₂CH₂O—), 3-aminopropoxy, and 4-aminobutoxy.

The term “carbonyl” refers to a C═O group.

The term “C₃-C₇ cycloalkyl” refers to a saturated ring radical consisting of 3 to 7 carbons. Some embodiments contain 3 to 4 carbons. Some embodiments contain 3 to 5 carbons. Some embodiments contain 4 to 6 carbons. Some embodiments contain 5 to 6 carbons. Examples include cyclopropyl, cyclobutyl, cyclopentyl, and cyclohexyl.

The term “C₄-C₁₃ cycloalkylalkyl” refers to a radical consisting of a C₃-C₇ cycloalkyl group attached to a C₁-C₆ alkyl group, wherein the C₃-C₇ cycloalkyl and C₁-C₆ alkyl groups have the same definitions as described herein. Examples include, but are not limited to, cyclopropylmethyl, cyclobutylmethyl, cyclopentylmethyl, cyclohexylmethyl, and cyclopropylethyl.

The term “carboxamide” refers to the group —CONH₂.

The term “carboxyl” refers to the group —CO₂H.

The term “cyano” refers to the group —CN.

The term “C₂-C₆ dialkylamino” refers to a radical consisting of an amino group substituted with two of the same or different C₁-C₃ alkyl groups, wherein C₁-C₃ alkyl has the same definition as found herein. Some examples include, but are not limited to, dimethylamino, methylethylamino, diethylamino, methylpropylamino, methylisopropylamino, ethylpropylamino, ethylisopropylamino, dipropylamino, and propylisopropylamino.

The term “C₂-C₆ dialkylcarboxamide” refers to a radical consisting of a carboxamide group, wherein either the nitrogen is substituted with two of the same or different C₁-C₃ alkyl groups, or the nitrogen and the carbonyl of the carboxamide group are each substituted with one C₁-C₃ alkyl group and can be the same or different, wherein C₁-C₃ alkyl has the same definition as found herein. The “C₂-C₆ dialkylcarboxamide” group can be represented by the following formulae:

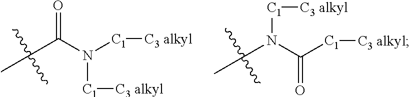

wherein C₁-C₃ alkyl has the same definition as found herein. Examples include, but are not limited to, N,N-dimethylcarboxamide, N,N-methylethylcarboxamide, and N,N-diethylcarboxamide.

The term “halogen” refers to a fluoro, chloro, bromo, or iodo group.

The term “heteroaryl” refers to a ring system consisting of 5 to 10 ring atoms, that may contain a single ring or two fused rings, and wherein at least one ring is aromatic and at least one ring atom is a heteroatom selected from, for example: O, S and N, wherein N is optionally substituted with H, C₁-C₄ acyl, C₁-C₄ alkyl, or O (i.e., forming an N-oxide) and S is optionally substituted with one or two oxygen atoms. In some embodiments, the aromatic ring contains one heteroatom. In some embodiments, the aromatic ring contains two heteroatoms. In some embodiments, the aromatic ring contains three heteroatoms. Some embodiments are directed to a heteroaryl selected from: furanyl, thienyl, pyrrolyl, imidazolyl, oxazolyl, thiazolyl, isoxazolyl, pyrazolyl, isothiazolyl, oxadiazolyl, triazolyl, tetrazolyl, thiadiazolyl, pyridinyl, pyrazinyl, pyrimidinyl, pyridazinyl, triazinyl, 4,5,6,7-tetrahydro-1H-imidazo[4,5-c]pyridinyl, and 5,6,7,8-tetrahydroimidazo[1,2-a]pyrazinyl. Some embodiments are directed to a heteroaryl selected from: 1H-imidazol-1-yl, 1H-1,2,4-triazol-1-yl, 1H-pyrazol-1-yl, pyridine-2-yl, 1H-pyrrol-1-yl, 2H-tetrazol-5-yl, 6,7-dihydro-1H-imidazo[4,5-c]pyridin-5(4H)-yl, and 5,6-dihydroimidazo[1,2-a]pyrazin-7(8H)-yl. Some embodiments are directed to 5-membered heteroaryl rings. Examples of a 5-membered heteroaryl ring include, but are not limited to, furanyl, thienyl, pyrrolyl, imidazolyl, oxazolyl, thiazolyl, isoxazolyl, pyrazolyl, isothiazolyl, oxadiazolyl, triazolyl, tetrazolyl, and thiadiazolyl. Some embodiments are directed to 6-membered heteroaryl rings. Examples of a 6-membered heteroaryl ring include, but are not limited to, pyridinyl, pyrazinyl, pyrimidinyl, pyridazinyl, triazinyl, and 2-oxo-1,2-dihydropyrimidinyl. Some embodiments are directed to heteroaryl rings that consist of two fused rings, examples include, but not limited to, 4,5,6,7-tetrahydro-1H-imidazo[4,5-c]pyridinyl, and 5,6,7,8-tetrahydroimidazo[1,2-a]pyrazinyl.

The term “heteroaryl-C₁-C₆-alkyl” refers to a radical consisting of a heteroaryl group attached to a C₁-C₆ alkyl group, wherein the heteroaryl and C₁-C₆ alkyl groups have the same definitions as described herein. Examples include, but are not limited to, 3-(imidazol-1-yl)propyl, 2-(pyridine-2-yl)ethyl, 2-(pyridine-3-yl)ethyl, 2-(imidazol-1-yl)ethyl, (imidazol-5-yl)ethyl, and (imidazol-5-yl)methyl.

The term “heterocyclyl” refers to a non-aromatic ring radical consisting of 3 to 10 ring atoms, wherein one, two or three ring atoms are heteroatoms selected independently from, for example: O, S, and N. It is understood that the sulfur atom can be optionally substituted with one or two oxo groups. The term encompasses spiro heterocyclyl, fused heterocyclyl, and bicyclic heterocyclyl groups. Examples of a heterocyclyl group include, but are not limited to, aziridinyl, azetidinyl, piperidinyl, morpholinyl, piperazinyl, pyrrolidinyl, [1,3]-dioxolanyl, thiomorpholinyl, [1,4]oxazepanyl, 1,1-dioxothiomorpholinyl, azepanyl, tetrahydrofuranyl, tetrahydropyranyl, tetrahydrothiopyranyl, 1-oxo-hexahydro-1λ⁴-thiopyranyl, 1,1-dioxo-hexahydro-1λ⁶-thiopyranyl, azabicyclo[3.2.1]octanyl, hexahydropyrrolo[1,2-a]pyrazinyl, 2,7-diazaspiro[4.4]nonanyl, 5,6-dihydropyrimidinyl, 2,3,4,6,7,8-hexahydro-1H-pyrimido[1,2-a]pyrimidinyl, 1,4-oxazepanyl, 2,5-diazabicyclo[2.2.1]heptanyl, 1,4-diazepanyl, and 2,7-diazaspiro[3.5]nonanyl. In some embodiments, heterocyclyl is selected from: thiomorpholin-4-yl, pyrrolidin-1-yl, piperazin-1-yl, hexahydropyrrolo[1,2-a]pyrazin-2(1H)-yl, piperidin-1-yl, morpholino, 2,7-diazaspiro[4.4]nonan-2-yl, 5,6-dihydropyrimidin-1(4H)-yl, 2,3,4,6,7,8-hexahydro-1H-pyrimido[1,2-a]pyrimidin-1-yl, 1,4-oxazepan-4-yl, azetidin-1-yl, 2,5-diazabicyclo[2.2.1]heptan-2-yl, 1,4-diazepan-1-yl, and 2,7-diazaspiro[3.5]nonan-2-yl.

The term “heterocyclyl-C₁-C₆-alkyl” refers to a radical consisting of a heterocyclyl group attached to a C₁-C₆ alkyl radical, wherein heterocyclyl and C₁-C₆ alkyl have the same definitions as described herein. Examples of a heterocyclyl-C₁-C₆-alkyl group include, but are not limited to, aziridinylmethyl, azetidinylmethyl, piperidinylmethyl, morpholinylmethyl, piperazinylmethyl, pyrrolidinylmethyl, [1,3]-dioxolanylmethyl, thiomorpholinylmethyl, [1,4]oxazepanylmethyl, 1,1-dioxothiomorpholinylmethyl, azepanylmethyl, tetrahydrofuranylmethyl, tetrahydropyranylmethyl, tetrahydrothiopyranylmethyl, 1-oxo-hexahydro-1λ⁴-thiopyranylmethyl, 1,1-dioxo-hexahydro-1λ⁶-thiopyranylmethyl, and azabicyclo[3.2.1]octanylmethyl.

The term “imino” refers to the diradical ═NH.

The term “isoindolinyl” refers to the group represented by the following formula:

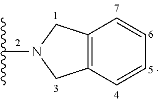 [SMILES: *N1Cc2ccccc2C1]

The term “hydroxy-C₁-C₆-alkyl” refers to a radical consisting of a hydroxyl group attached to a C₁-C₆ alkyl radical, wherein hydroxyl and C₁-C₆ alkyl have the same definitions as described herein. Examples include, but are not limited to hydroxymethyl, 2-hydroxyethyl, and 1-hydroxyethyl.

The term “hydroxyl” refers to the radical —OH.

The term “hydroxyheterocyclyl” refers to a radical consisting of a hydroxyl group attached to a heterocyclyl radical, wherein hydroxyl and heterocyclyl have the same definitions as described herein. Examples include, but are not limited to 3-hydroxyazetidin-1-yl, 3-hydroxypyrrolidin-1-yl, 3-hydroxypiperidin-1-yl, and 4-hydroxypiperidin-1-yl.

The term “oxo” refers to the diradical ═O.

The term “phenyl” refers to the group —C₆H₅.

The term “phosphonooxy” refers to the radical —OPO₃H₂.

The term “1,2,3,4-tetrahydroisoquinolinyl” refers to the group represented by the following formula:

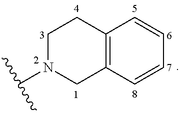 [SMILES: *N1CCc2ccccc2C1]

### Compounds of the Present Invention

One aspect of the present invention encompasses, inter alia, certain amide derivatives selected from compounds of Formula (I) and pharmaceutically acceptable salts, solvates, and hydrates thereof:

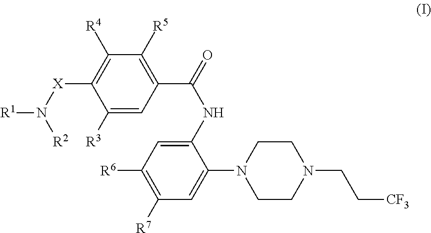

wherein R¹, R², R³, R⁴, R⁵, R⁶, R⁷, and “X”, have the same definitions as described herein.

One aspect of the present invention is directed to compounds of Formula (Ia) and pharmaceutically acceptable salts, solvates, and hydrates thereof:

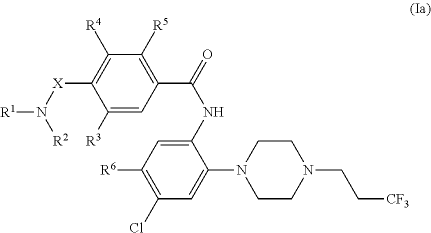

wherein R¹, R², R³, R⁴, R⁵, R⁶, and “X”, have the same definitions as described herein.

One aspect of the present invention is directed to compounds of Formula (Ic) and pharmaceutically acceptable salts, solvates, and hydrates thereof:

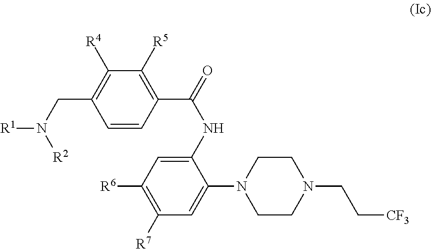

wherein R¹, R², R⁴, R⁵, R⁶, and R⁷, have the same definitions as described herein.

One aspect of the present invention is directed to compounds of Formula (Ie) and pharmaceutically acceptable salts, solvates, and hydrates thereof:

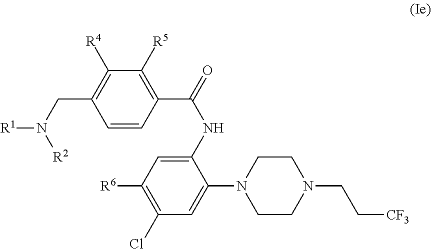

wherein R¹, R², R⁴, R⁵, and R⁶, have the same definitions as described herein.

One aspect of the present invention is directed to compounds of Formula (Ig) and pharmaceutically acceptable salts, solvates, and hydrates thereof:

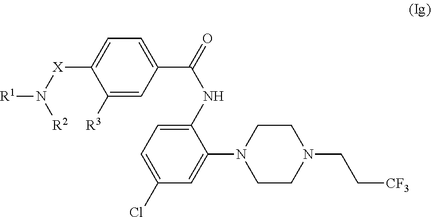

wherein R¹, R², R³, and “X”, have the same definitions as described herein.

One aspect of the present invention is directed to compounds of Formula (Ii) and pharmaceutically acceptable salts, solvates, and hydrates thereof:

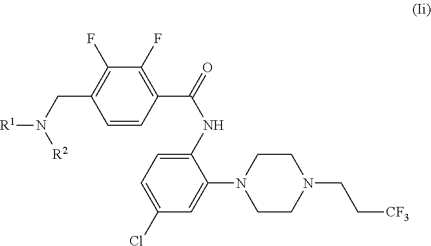

wherein R¹ and R², have the same definitions as described herein.

One aspect of the present invention is directed to compounds of Formula (Ik) and pharmaceutically acceptable salts, solvates, and hydrates thereof:

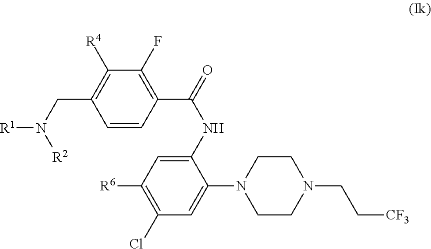

wherein R¹, R², R⁴, and R⁶, have the same definitions as described herein.

Some embodiments are directed to compounds and pharmaceutically acceptable salts, solvates, and hydrates of the present invention provided that the nitrogen atom bonded to R¹, R², and X is not directly bonded to a carbonyl group.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables (e.g., R¹, R², R³, R⁴, R⁵, R⁶, R⁷, and “X”) contained within the generic chemical formulae described herein are specifically embraced by the present invention just as if each and every combination was individually and explicitly recited, to the extent that such combinations embrace compounds that result in stable compounds (i.e., compounds that can be isolated, characterized, and tested for biological activity). In addition, all subcombinations of the chemical groups listed in the embodiments describing such variables, as well as all subcombinations of uses and medical indications described herein, are also specifically embraced by the present invention just as if each and every subcombination of chemical groups and subcombination of uses and medical indications was individually and explicitly recited herein.

As used herein, “substituted” indicates that at least one hydrogen atom of the chemical group is replaced by a non-hydrogen substituent or group, which can be monovalent or divalent. When the substituent or group is divalent, then it is understood that this group can be further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents, and the like. Likewise, “substituted with one or more substituents” refers to the substitution of a group with one substituent up to the total number of substituents physically allowed by the group. Further, when a group is substituted with more than one group they can be identical or they can be different.

Compounds of the invention can also include tautomeric forms, such as keto-enol tautomers and the like. Tautomeric forms can be in equilibrium or sterically locked into one form by appropriate substitution. It is understood that the various tautomeric forms are within the scope of the compounds of the present invention.

It is understood and appreciated that compounds of Formula (I) and formulae related thereto may have one or more chiral centers and therefore can exist as enantiomers and/or diastereoisomers. The invention is understood to extend to and embrace all such enantiomers, diastereoisomers and mixtures thereof, including but not limited to racemates. It is understood that compounds of Formula (I) and formulae used throughout this disclosure represent all individual enantiomers and mixtures thereof, unless stated or shown otherwise.

### the Group X

In some embodiments, X is CH₂ or CH₂CH₂; or X is absent.

In some embodiments, X is CH₂ or CH₂CH₂.

In some embodiments, X is CH₂.

In some embodiments, X is CH₂CH₂.

In some embodiments, X is absent.

### The Group R¹

In some embodiments, R¹ is selected from: H, C₁-C₆ alkyl, C₁-C₆-alkyl-O—C₁-C₆-alkyl, C₃-C₇ cycloalkyl, C₄-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy.

In some embodiments, R¹ is selected from: H, C₁-C₆ alkyl, C₁-C₆-alkyl-O—C₁-C₆-alkyl, C₄-C₁₃ cycloalkylalkyl, heteroaryl-C₁-C₆-alkyl, and heterocyclyl-C₁-C₆-alkyl, each optionally substituted with one or more substituents selected from: C₁-C₆ alkoxycarbonyl, C₁-C₆ alkylsulfonyl, carboxamide, cyano, C₂-C₆ dialkylamino, hydroxyl, and oxo.

In some embodiments, R¹ is selected from: H, C₁-C₆ alkyl, C₁-C₆-alkyl-O—C₁-C₆-alkyl, and heterocyclyl-C₁-C₆-alkyl, each optionally substituted with one or more substituents selected from: C₁-C₆ alkoxycarbonyl, C₁-C₆ alkylsulfonyl, carboxamide, cyano, C₂-C₆ dialkylamino, and hydroxyl.

In some embodiments, R¹ is selected from: H, C₁-C₆ alkyl, C₁-C₆-alkyl-O—C₁-C₆-alkyl, C₃-C₇ cycloalkyl, C₄-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: tert-butoxycarbonylamino, 2-aminoethoxy, methoxycarbonyl, tert-butoxycarbonyl, methyl, ethyl, propan-1-yl, 3,3-dimethylbutyl, acetamido, methylsulfinyl, amino, carboxamide, carboxyl, cyano, dimethylamino, diethylamino, hydroxyl, hydroxymethyl, 2-hydroxyethyl, 3-hydroxypropyl, imino, oxo, phenyl, and phosphonooxy.

In some embodiments, R¹ is selected from: H, C₁-C₆ alkyl, C₁-C₆-alkyl-O—C₁-C₆-alkyl, C₃-C₇ cycloalkyl, C₄-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: tert-butoxycarbonylamino, 2-aminoethoxy, methoxycarbonyl, methyl, ethyl, acetamido, methylsulfinyl, amino, carboxamide, carboxyl, cyano, dimethylamino, diethylamino, hydroxyl, hydroxymethyl, imino, oxo, phenyl, and phosphonooxy.

In some embodiments, R¹ is selected from: H, C₁-C₆ alkyl, C₁-C₆-alkyl-O—C₁-C₆-alkyl, C₄-C₁₃ cycloalkylalkyl, heteroaryl-C₁-C₆-alkyl, and heterocyclyl-C₁-C₆-alkyl, each optionally substituted with one or more substituents selected from: ethoxycarbonyl, methylsulfonyl, carboxamide, cyano, diethylamino, hydroxyl, and oxo.

In some embodiments, R¹ is selected from: H, C₁-C₆ alkyl, C₁-C₆-alkyl-O—C₁-C₆-alkyl, and heterocyclyl-C₁-C₆-alkyl, each optionally substituted with one or more substituents selected from: ethoxycarbonyl, methylsulfonyl, carboxamide, cyano, diethylamino, and hydroxyl.

In some embodiments, R¹ is selected from: H, ethyl, methyl, 4-methylpentan-2-yl, propan-2-yl, propan-1-yl, tert-butyl, butan-2-yl, 3-methylpentan-2-yl, pentan-1-yl, butan-1-yl, isobutyl, isopentyl, 2-ethoxyethyl, cyclohexyl, cyclopentyl, (cyclohexyl)methyl, pyridin-3-yl, 3-(1H-imidazol-1-yl)propyl, 2-(1H-imidazol-5-yl)ethyl, (1H-imidazol-5-yl)methyl, 2-(pyridin-3-yl)ethyl, (2H-tetrazol-5-yl)methyl, tetrahydrothiophen-3-yl, azepan-3-yl, pyrrolidin-3-yl, piperidin-3-yl, piperidin-4-yl, (piperidin-4-yl)methyl, 2-(imidazolidin-1-yl)ethyl, (pyrrolidin-2-yl)methyl, 2-(piperazin-1-yl)ethyl, 2-(pyrrolidin-2-yl)ethyl, 2-morpholinoethyl, (tetrahydrothiophen-3-yl)methyl, 2-(pyrrolidin-1-yl)ethyl, 2-methyl-(2-(piperidin-1-yl))propan-1-yl, 2-(azepan-1-yl)ethyl, (2,3-dihydroisoxazol-5-yl)methyl, piperidin-4-ylmethyl, morpholin-2-ylmethyl, pyrrolidin-2-ylmethyl, thiomorpholin-3-ylmethyl, 2-(morpholin-4-yl)ethyl, 2-(1H-tetrazol-5-yl)ethyl, (2,3-dihydroisoxazol-5-yl)methyl, (1,6-dihydropyridazin-3-yl)methyl, (pyrimidin-5-yl)methyl, (4,5-dihydro-1H-1,2,4-triazol-3-yl)methyl, (tetrahydro-2H-thiopyran-4-yl)methyl, pyridin-3-ylmethyl, pyridin-4-ylmethyl, (1,2,3,6-tetrahydropyrimidin-4-yl)methyl, (1H-pyrazol-3-yl)methyl, 3-(isoxazol-4-yl)propyl, pyrazin-2-ylmethyl, pyridin-2-ylmethyl, pentyl, cyclopropylmethyl, butyl, neopentyl, 2-ethylbutyl, 4-methylpentyl, 2-(piperidin-2-yl)ethyl, 2-(pyrrolidin-3-yl)ethyl, 3-(piperidin-1-yl)propyl, pyrrolidin-3-ylmethyl, 2-cyclohexylethyl, 3-morpholinopropyl, cyclopentylmethyl, piperidin-3-ylmethyl, 2-(piperidin-3-yl)ethyl, azetidin-3-ylmethyl, 2-(piperidin-1-yl)ethyl, 2-(1,4-diazepan-1-yl)ethyl, 2-(2,5-diazabicyclo[2.2.1]heptan-2-yl)ethyl, 2-(hexahydropyrrolo[1,2-a]pyrazin-2(1H)-yl)ethyl, pyrrolidin-1-ylmethyl, (1,1-dioxotetrahydro-2H-thiopyran-4-yl)methyl, (1H-1,2,3-triazol-4-yl)methyl, piperazin-1-ylmethyl, piperidin-1-ylmethyl, azetidin-1-ylmethyl, and (morpholin-4-yl)methyl; each optionally substituted with one or more substituents selected from: tert-butoxycarbonylamino, 2-aminoethoxy, methoxycarbonyl, tert-butoxycarbonyl, methyl, ethyl, propan-1-yl, 3,3-dimethylbutyl, acetamido, methylsulfinyl, amino, carboxamide, carboxyl, cyano, dimethylamino, diethylamino, hydroxyl, hydroxymethyl, 2-hydroxyethyl, 3-hydroxypropyl, imino, oxo, phenyl, and phosphonooxy.

In some embodiments, R¹ is selected from: H, ethyl, methyl, 4-methylpentan-2-yl, propan-2-yl, propan-1-yl, tert-butyl, butan-2-yl, 3-methylpentan-2-yl, pentan-1-yl, butan-1-yl, isobutyl, isopentyl, 2-ethoxyethyl, cyclohexyl, cyclopentyl, (cyclohexyl)methyl, pyridin-3-yl, 3-(1H-imidazol-1-yl)propyl, 2-(1H-imidazol-5-yl)ethyl, (1H-imidazol-5-yl)methyl, 2-(pyridin-3-yl)ethyl, (2H-tetrazol-5-yl)methyl, tetrahydrothiophen-3-yl, azepan-3-yl, pyrrolidin-3-yl, piperidin-3-yl, piperidin-4-yl, (piperidin-4-yl)methyl, 2-(imidazolidin-1-yl)ethyl, (pyrrolidin-2-yl)methyl, 2-(piperazin-1-yl)ethyl, 2-(pyrrolidin-2-yl)ethyl, 2-(morpholino)ethyl, (tetrahydrothiophen-3-yl)methyl, 2-(pyrrolidin-1-yl)ethyl, 2-methyl-(2-(piperidin-1-yl))propan-1-yl, 2-(azepan-1-yl)ethyl, and (2,3-dihydroisoxazol-5-yl)methyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy.

In some embodiments, R¹ is selected from: H, methyl, butan-1-yl, propan-1-yl, 3,3-dimethylbutyl, ethyl, 2-methoxyethyl, 2-ethoxyethyl, (tetrahydro-2H-pyran-4-yl)methyl, 2-ethylbutyl, thiophen-2-ylmethyl, pyridin-3-ylmethyl, and 2-cyclopentylethyl, each optionally substituted with one or more substituents selected from: C₁-C₆ alkoxycarbonyl, C₁-C₆ alkylsulfonyl, carboxamide, cyano, C₂-C₆ dialkylamino, hydroxyl, and oxo.

In some embodiments, R¹ is selected from: H, methyl, butan-1-yl, propan-1-yl, 3,3-dimethylbutyl, ethyl, 2-methoxyethyl, 2-ethoxyethyl, and (tetrahydro-2H-pyran-4-yl)methyl, each optionally substituted with one or more substituents selected from: C₁-C₆ alkoxycarbonyl, C₁-C₆ alkylsulfonyl, carboxamide, cyano, C₂-C₆ dialkylamino, and hydroxyl.

In some embodiments, R¹ is selected from: H, ethyl, methyl, 4-methylpentan-2-yl, propan-2-yl, propan-1-yl, tert-butyl, butan-2-yl, 3-methylpentan-2-yl, pentan-1-yl, butan-1-yl, isobutyl, isopentyl, 2-ethoxyethyl, cyclohexyl, cyclopentyl, (cyclohexyl)methyl, pyridin-3-yl, 3-(1H-imidazol-1-yl)propyl, 2-(1H-imidazol-5-yl)ethyl, (1H-imidazol-5-yl)methyl, 2-(pyridin-3-yl)ethyl, (2H-tetrazol-5-yl)methyl, tetrahydrothiophen-3-yl, azepan-3-yl, pyrrolidin-3-yl, piperidin-3-yl, piperidin-4-yl, (piperidin-4-yl)methyl, 2-(imidazolidin-1-yl)ethyl, (pyrrolidin-2-yl)methyl, 2-(piperazin-1-yl)ethyl, 2-(pyrrolidin-2-yl)ethyl, 2-(morpholino)ethyl, (tetrahydrothiophen-3-yl)methyl, 2-(pyrrolidin-1-yl)ethyl, 2-methyl-(2-(piperidin-1-yl))propan-1-yl, 2-(azepan-1-yl)ethyl, and (2,3-dihydroisoxazol-5-yl)methyl, each optionally substituted with one or more substituents selected from: tert-butoxycarbonylamino, 2-aminoethoxy, methoxycarbonyl, methyl, ethyl, acetamido, methylsulfinyl, amino, carboxamide, carboxyl, cyano, dimethylamino, diethylamino, hydroxyl, hydroxymethyl, imino, oxo, phenyl, and phosphonooxy.

In some embodiments, R¹ is selected from: H, methyl, butan-1-yl, propan-1-yl, 3,3-dimethylbutyl, ethyl, 2-methoxyethyl, 2-ethoxyethyl, (tetrahydro-2H-pyran-4-yl)methyl, 2-ethylbutyl, thiophen-2-ylmethyl, pyridin-3-ylmethyl, and 2-cyclopentylethyl, each optionally substituted with one or more substituents selected from: ethoxycarbonyl, methylsulfonyl, carboxamide, cyano, diethylamino, hydroxyl, and oxo.

In some embodiments, R¹ is selected from: H, methyl, butan-1-yl, propan-1-yl, 3,3-dimethylbutyl, ethyl, 2-methoxyethyl, 2-ethoxyethyl, and (tetrahydro-2H-pyran-4-yl)methyl, each optionally substituted with one or more substituents selected from: ethoxycarbonyl, methylsulfonyl, carboxamide, cyano, diethylamino, and hydroxyl.

In some embodiments, R¹ is selected from: H, ethyl, 2-hydroxyethyl, 3-(1H-imidazol-1-yl)propyl, 4-methylpyridin-3-yl, methyl, 2-cyanoethyl, 2-amino-2-oxoethylamino, (1-methylpiperidin-4-yl)methyl, cyanomethyl, 1-amino-1-oxopropan-2-yl, 1,1-dioxo-tetrahydrothiophen-3-yl, 1-hydroxy-4-methylpentan-2-yl, 2-(1H-imidazol-5-yl)ethyl, (1-methyl-1H-imidazol-5-yl)methyl, 2-carbamoylcyclohexyl, 3-hydroxy-1-methoxy-1-oxopropan-2-yl, 1,3-dihydroxypropan-2-yl, 1-amino-3-hydroxy-1-oxopropan-2-yl, 2-hydroxycyclohexyl, 2-oxoazepan-3-yl, 2-(2-oxoimidazolidin-1-yl)ethyl, pyrrolidin-2-ylmethyl, pyrrolidin-3-yl, piperidin-3-yl, piperidin-4-yl, 2-hydroxypropyl, 2-hydroxypyridin-3-yl, 2-(4-methylpiperazin-1-yl)ethyl, 1-hydroxypropan-2-yl, 1,3-dihydroxy-2-(hydroxymethyl)propan-2-yl, 2-acetamidoethyl, 1-hydroxybutan-2-yl, 2-(1-methylpyrrolidin-2-yl)ethyl, 2-(dimethylamino)ethyl, 2-morpholinoethyl, 1-ethyl-2-oxoazepan-3-yl, 3-(dimethylamino)tetrahydrothiophen-3-yl)methyl, 2-(diethylamino)ethyl, 1-hydroxy-3-methylpentan-2-yl, 5-aminopentyl, 3-amino-1-imino-3-oxopropyl, (1-hydroxycyclohexyl)methyl, 2-(hydroxymethyl)pyrrolidin-1-yl)ethyl, 2-methyl-2-(piperidin-1-yl)propyl, benzyl, 2-(methylsulfinyl)ethyl, 2-(azepan-1-yl)ethyl, 3-hydroxybutyl, 1-amino-3-methyl-1-oxobutan-2-yl, 2-(2-(2-aminoethoxy)ethoxy)ethyl, 2-(hydroxymethyl)pyrrolidin-1-yl, 1,3-dihydroxybutan-2-yl, 2-morpholino-2-oxoethyl, 2-(dimethylamino)-2-(pyridin-3-yl)ethyl, 2-(pyrrolidin-1-yl)ethyl, 3-amino-1-methoxy-1-oxopropan-2-yl, 4-amino-1-methoxy-1-oxobutan-2-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, 2-hydroxycyclopentyl, piperidine-4-carbonyl, 2-aminocyclohexanecarbonyl, morpholine-2-carbonyl, 3-aminopropanoyl, 2-aminoacetyl, 4-hydroxypyrrolidine-2-carbonyl, 2-aminopropanoyl, 2-amino-3-hydroxypropanoyl, 2-hydroxyacetyl, thiomorpholine-3-carbonyl, pyrrolidine-2-carbonyl, 2-(morpholin-4-yl)acetyl, 2-(1H-tetrazol-5-yl)acetyl, 2-(dimethylamino)acetyl, 3-oxo-2,3-dihydroisoxazole-5-carbonyl, 6-oxo-1,6-dihydropyridazine-3-carbonyl, 2,4-dihydroxypyrimidine-5-carbonyl, 5-oxo-4,5-dihydro-1H-1,2,4-triazole-3-carbonyl, 4-aminotetrahydro-2H-thiopyran-4-carbonyl, 2-(3-amino-2-oxopyrrolidin-1-yl)acetyl, 6-hydroxynicotinoyl, 2-hydroxynicotinoyl, 2,6-dihydroxyisonicotinoyl, 2,6-dioxo-1,2,3,6-tetrahydropyrimidine-4-carbonyl, 5-hydroxy-1-methyl-1H-pyrazole-3-carbonyl, 3-(3-hydroxyisoxazol-4-yl)propanoyl, 3-carboxypropanoyl, 5-hydroxypyrazine-2-carbonyl, 6-hydroxypicolinoyl, 4-methylmorpholine-2-carbonyl, 4-ethylmorpholine-2-carbonyl, 4-(2-hydroxyethyl)morpholine-2-carbonyl, 4-(3,3-dimethylbutyl)morpholine-2-carbonyl, 4-(2-hydroxyethyl)morpholine-3-carbonyl, 4-ethylmorpholine-3-carbonyl, 4-(2-hydroxyethyl)thiomorpholine-3-carbonyl, 4-ethylthiomorpholine-3-carbonyl, 3-hydroxypropanoyl, 4-hydroxycyclohexanecarbonyl, 3-hydroxypentanoyl, 2-hydroxy-2-methylpropanoyl, 1-hydroxycyclopropanecarbonyl, 3-hydroxybutanoyl, 3-hydroxy-2,2-dimethylpropanoyl, 4-hydroxybutanoyl, 2-ethyl-2-hydroxybutanoyl, 2-hydroxycyclohexanecarbonyl, 2-cyclohexyl-2-hydroxyacetyl, 3-hydroxy-3-methylbutanoyl, 2-hydroxy-4-methylpentanoyl, 1-(tert-butoxycarbonyl)-4-hydroxypyrrolidine-2-carbonyl, 4-(tert-butoxycarbonyl)thiomorpholine-3-carbonyl, 2-(1-(tert-butoxycarbonyl)piperidin-2-yl)acetyl, 3-hydroxy-2-(hydroxymethyl)-2-methylpropanoyl, 2-(piperidin-2-yl)acetyl, 4-(hydroxymethyl)cyclohexanecarbonyl, 3-(dimethylamino)propanoyl, 2-(pyrrolidin-3-yl)acetyl, 3-(piperidin-1-yl)propanoyl, 4-aminocyclohexanecarbonyl, pyrrolidine-3-carbonyl, 3-(diethylamino)propanoyl, 2-(4-aminocyclohexyl)acetyl, 3-morpholinopropanoyl, 1-methylpiperidine-4-carbonyl, 3-aminocyclohexanecarbonyl, 2-amino-4-carboxybutanoyl, 4-amino-4-carboxybutanoyl, 3-aminocyclopentanecarbonyl, 1-methylpiperidine-3-carbonyl, 2-(piperidin-3-yl)acetyl, azetidine-3-carbonyl, 2-(4-(hydroxymethyl)piperidin-1-yl)acetyl, 2-(3-hydroxypiperidin-1-yl)acetyl, 2-(piperazin-1-yl)acetyl, 2-(3-aminopyrrolidin-1-yl)acetyl, 2-(2-(hydroxymethyl)morpholino)acetyl, 2-(4-propylpiperazin-1-yl)acetyl, 2-(5-oxo-1,4-diazepan-1-yl)acetyl, 2-(4-carbamoylpiperidin-1-yl)acetyl, 2-(2-carbamoylpyrrolidin-1-yl)acetyl, 2-(4-(dimethylamino)piperidin-1-yl)acetyl, 2-(3-(dimethylamino)pyrrolidin-1-yl)acetyl, 2-(4-hydroxypiperidin-1-yl)acetyl, 2-(2,5-diazabicyclo[2.2.1]heptan-2-yl)acetyl, 2-(hexahydropyrrolo[1,2-a]pyrazin-2(1H)-yl)acetyl, 2-(3-(hydroxymethyl)piperidin-1-yl)acetyl, 2-(3-methylpiperazin-1-yl)acetyl, 2-(4-methylpiperidin-1-yl)acetyl, 2-(3-oxopiperazin-1-yl)acetyl, 2-(4-carbamoylpiperazin-1-yl)acetyl, 2-(3-methylpiperidin-1-yl)acetyl, 2-(4-methylpiperazin-1-yl)acetyl, 2-(4-ethylpiperazin-1-yl)acetyl, 2-(2-(2-hydroxyethyl)piperidin-1-yl)acetyl, 2-(3-hydroxypyrrolidin-1-yl)acetyl, 2-(2-(hydroxymethyl)pyrrolidin-1-yl)acetyl, 2-(3-carbamoylpiperidin-1-yl)acetyl, 4-(phosphonooxy)cyclohexanecarbonyl, 2-(phosphonooxy)acetyl, 3-(tert-butoxycarbonylamino)pyrrolidine-1-carbonyl, 2-amino-4-methylpentanoyl, 2-amino-3-cyanopropanoyl, 4-amino-1,1-dioxotetrahydro-2H-thiopyran-4-carbonyl, 2,4-diamino-4-oxobutanoyl, 3-amino-2-hydroxypropanoyl, 2-hydroxypropanoyl, 5-(hydroxymethyl)-1H-1,2,3-triazole-4-carbonyl, piperazine-1-carbonyl, 4-ethylpiperazine-1-carbonyl, 1,1-dioxotetrahydro-2H-thiopyran-4-carbonyl, 3-hydroxypyrrolidine-1-carbonyl, 4-(2-hydroxyethyl)piperazine-1-carbonyl, 4-(hydroxymethyl)piperidine-1-carbonyl, 3-aminopiperidine-1-carbonyl, 3-hydroxyazetidine-1-carbonyl, 3-aminopyrrolidine-1-carbonyl, 2-carbamoylpyrrolidine-1-carbonyl, 4-(dimethylamino)piperidine-1-carbonyl, 4-carbamoylpiperazine-1-carbonyl, 3-oxopiperazine-1-carbonyl, 2-(hydroxymethyl)pyrrolidine-1-carbonyl, 2-(2-hydroxyethyl)piperidine-1-carbonyl, 2-(hydroxymethyl)morpholine-4-carbonyl, 3-carboxyazetidine-1-carbonyl, 4-(3-hydroxypropyl)piperidine-1-carbonyl, 3-hydroxypiperidine-1-carbonyl, 4-cyanopiperidine-1-carbonyl, 2-(hydroxymethyl)piperidine-1-carbonyl, 4-hydroxypiperidine-1-carbonyl, 2-oxopyrrolidine-1-carbonyl, 3-(hydroxymethyl)piperidine-1-carbonyl, 3-(hydroxymethyl)pyrrolidine-1-carbonyl, 3-(phosphonooxy)pyrrolidine-1-carbonyl, 1-(tert-butoxycarbonyl)piperidine-4-carbonyl, 2-(tert-butoxycarbonylamino)-cyclohexanecarbonyl, 1-(tert-butoxycarbonyl)piperidine-3-carbonyl, 3-(tert-butoxycarbonylamino)piperidine-1-carbonyl, 4-(tert-butoxycarbonyl)morpholine-2-carbonyl, 3-(tert-butoxycarbonylamino)propanoyl, 2-(tert-butoxycarbonylamino)acetyl, 3-(tert-butoxycarbonylamino)-2-hydroxypropanoyl, 2-(tert-butoxycarbonylamino)propanoyl, 2-(tert-butoxycarbonylamino)-3-hydroxypropanoyl, 1-(tert-butoxycarbonyl)pyrrolidine-2-carbonyl, 4-(tert-butoxycarbonylamino)tetrahydro-2H-thiopyran-4-carbonyl, 4-tert-butoxy-4-oxobutanoyl, 2-(3-(tert-butoxycarbonylamino)-2-oxopyrrolidin-1-yl)acetyl, 4-amino-2-(tert-butoxycarbonylamino)-4-oxobutanoyl, 2-(1-(tert-butoxycarbonyl)pyrrolidin-3-yl)acetyl, 4-(tert-butoxycarbonylamino)cyclohexanecarbonyl, 1-(tert-butoxycarbonyl)pyrrolidine-3-carbonyl, 2-(4-(tert-butoxycarbonylamino)cyclohexyl)acetyl, 3-(tert-butoxycarbonylamino)cyclohexanecarbonyl, 2-(tert-butoxycarbonylamino)-4-carboxybutanoyl, 4-(tert-butoxycarbonylamino)-4-carboxybutanoyl, 3-(tert-butoxycarbonylamino)cyclopentanecarbonyl, 2-(1-(tert-butoxycarbonyl)piperidin-3-yl)acetyl, 1-(tert-butoxycarbonyl)azetidine-3-carbonyl, 2-(3-(tert-butoxycarbonylamino)pyrrolidin-1-yl, 2-(4-(tert-butoxycarbonyl)-3-methylpiperazin-1-yl)acetyl, 2-(tert-butoxycarbonylamino)-4-methylpentanoyl, 2-(tert-butoxycarbonylamino)-3-cyanopropanoyl, and 4-(tert-butoxycarbonylamino)-1,1-dioxotetrahydro-2H-thiopyran-4-carbonyl.

In some embodiments, R¹ is selected from: H, ethyl, 2-hydroxyethyl, 3-(1H-imidazol-1-yl)propyl, 4-methylpyridin-3-yl, methyl, 2-cyanoethyl, 2-amino-2-oxoethylamino, (1-methylpiperidin-4-yl)methyl, cyanomethyl, 1-amino-1-oxopropan-2-yl, 1,1-dioxo-tetrahydrothiophen-3-yl, 1-hydroxy-4-methylpentan-2-yl, 2-(1H-imidazol-5-yl)ethyl, (1-methyl-1H-imidazol-5-yl)methyl, 2-carbamoylcyclohexyl, 3-hydroxy-1-methoxy-1-oxopropan-2-yl, 1,3-dihydroxypropan-2-yl, 1-amino-3-hydroxy-1-oxopropan-2-yl, 2-hydroxycyclohexyl, 2-oxoazepan-3-yl, 2-(2-oxoimidazolidin-1-yl)ethyl, pyrrolidin-2-ylmethyl, pyrrolidin-3-yl, piperidin-3-yl, piperidin-4-yl, 2-hydroxypropyl, 2-hydroxypyridin-3-yl, 2-(4-methylpiperazin-1-yl)ethyl, 1-hydroxypropan-2-yl, 1,3-dihydroxy-2-(hydroxymethyl)propan-2-yl, 2-acetamidoethyl, 1-hydroxybutan-2-yl, 2-(1-methylpyrrolidin-2-yl)ethyl, 2-(dimethylamino)ethyl, 2-morpholinoethyl, 1-ethyl-2-oxoazepan-3-yl, 3-(dimethylamino)tetrahydrothiophen-3-yl)methyl, 2-(diethylamino)ethyl, 1-hydroxy-3-methylpentan-2-yl, 5-aminopentyl, 3-amino-1-imino-3-oxopropyl, (1-hydroxycyclohexyl)methyl, 2-(hydroxymethyl)pyrrolidin-1-yl)ethyl, 2-methyl-2-(piperidin-1-yl)propyl, benzyl, 2-(methylsulfinyl)ethyl, 2-(azepan-1-yl)ethyl, 3-hydroxybutyl, 1-amino-3-methyl-1-oxobutan-2-yl, 2-(2-(2-aminoethoxy)ethoxy)ethyl, 2-(hydroxymethyl)pyrrolidin-1-yl, 1,3-dihydroxybutan-2-yl, 2-morpholino-2-oxoethyl, 2-(dimethylamino)-2-(pyridin-3-yl)ethyl, 2-(pyrrolidin-1-yl)ethyl, 3-amino-1-methoxy-1-oxopropan-2-yl, 4-amino-1-methoxy-1-oxobutan-2-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, and 2-hydroxycyclopentyl.

In some embodiments, R¹ is selected from: H, methyl, butyl, 3-hydroxypropyl, 3,3-dimethylbutyl, (tetrahydro-2H-pyran-4-yl)methyl, 2-methoxyethyl, 3-amino-3-oxopropyl, 2-hydroxyethyl, 2-ethoxy-2-oxoethyl, 2-amino-2-oxoethyl, cyanomethyl, 2-ethoxyethyl, 2-(diethylamino)ethyl, 2-(methylsulfonyl)ethyl, butyr-1-yl, 2-ethylbutanoyl, thiophene-2-carbonyl, nicotinoyl, and 2-cyclopentylacetyl.

In some embodiments, R¹ is selected from: H, methyl, butyl, 3-hydroxypropyl, 3,3-dimethylbutyl, (tetrahydro-2H-pyran-4-yl)methyl, 2-methoxyethyl, 3-amino-3-oxopropyl, 2-hydroxyethyl, 2-ethoxy-2-oxoethyl, 2-amino-2-oxoethyl, cyanomethyl, 2-ethoxyethyl, 2-(diethylamino)ethyl, and 2-(methylsulfonyl)ethyl.

In some embodiments, R¹ is H. In some embodiments, R¹ is ethyl. In some embodiments, R¹ is 2-hydroxyethyl. In some embodiments, R¹ is 3-(1H-imidazol-1-yl)propyl. In some embodiments, R¹ is 4-methylpyridin-3-yl. In some embodiments, R¹ is methyl. In some embodiments, R¹ is 2-cyanoethyl. In some embodiments, R¹ is 2-amino-2-oxoethylamino. In some embodiments, R¹ is (1-methylpiperidin-4-yl)methyl. In some embodiments, R¹ is cyanomethyl. In some embodiments, R¹ is 1-amino-1-oxopropan-2-yl. In some embodiments, R¹ is 1,1-dioxo-tetrahydrothiophen-3-yl. In some embodiments, R¹ is 1-hydroxy-4-methylpentan-2-yl. In some embodiments, R¹ is 2-(1H-imidazol-5-yl)ethyl. In some embodiments, R¹ is (1-methyl-1H-imidazol-5-yl)methyl. In some embodiments, R¹ is 2-carbamoylcyclohexyl. In some embodiments, R¹ is 3-hydroxy-1-methoxy-1-oxopropan-2-yl. In some embodiments, R¹ is 1,3-dihydroxypropan-2-yl. In some embodiments, R¹ is 1-amino-3-hydroxy-1-oxopropan-2-yl. In some embodiments, R¹ is 2-hydroxycyclohexyl. In some embodiments, R¹ is 2-oxoazepan-3-yl.

In some embodiments, R¹ is 2-(2-oxoimidazolidin-1-yl)ethyl. In some embodiments, R¹ is pyrrolidin-2-ylmethyl. In some embodiments, R¹ is pyrrolidin-3-yl. In some embodiments, R¹ is piperidin-3-yl. In some embodiments, R¹ is piperidin-4-yl. In some embodiments, R¹ is 2-hydroxypropyl. In some embodiments, R¹ is 2-hydroxypyridin-3-yl. In some embodiments, R¹ is 2-(4-methylpiperazin-1-yl)ethyl. In some embodiments, R¹ is 1-hydroxypropan-2-yl. In some embodiments, R¹ is 1,3-dihydroxy-2-(hydroxymethyl)propan-2-yl. In some embodiments, R¹ is 2-acetamidoethyl. In some embodiments, R¹ is 1-hydroxybutan-2-yl. In some embodiments, R¹ is 2-(1-methylpyrrolidin-2-yl)ethyl. In some embodiments, R¹ is 2-(dimethylamino)ethyl. In some embodiments, R¹ is 2-morpholinoethyl. In some embodiments, R¹ is 1-ethyl-2-oxoazepan-3-yl. In some embodiments, R¹ is 3-(dimethylamino)tetrahydrothiophen-3-yl)methyl. In some embodiments, R¹ is 2-(diethylamino)ethyl. In some embodiments, R¹ is 1-hydroxy-3-methylpentan-2-yl. In some embodiments, R¹ is 5-aminopentyl. In some embodiments, R¹ is 3-amino-1-imino-3-oxopropyl. In some embodiments, R¹ is (1-hydroxycyclohexyl)methyl. In some embodiments, R¹ is 2-(hydroxymethyl)pyrrolidin-1-yl)ethyl. In some embodiments, R¹ is 2-methyl-2-(piperidin-1-yl)propyl. In some embodiments, R¹ is benzyl. In some embodiments, R¹ is 2-(methylsulfinyl)ethyl. In some embodiments, R¹ is 2-(azepan-1-yl)ethyl. In some embodiments, R¹ is 3-hydroxybutyl. In some embodiments, R¹ is 1-amino-3-methyl-1-oxobutan-2-yl. In some embodiments, R¹ is 2-(2-(2-aminoethoxy)ethoxy)ethyl. In some embodiments, R¹ is 2-(hydroxymethyl)pyrrolidin-1-yl. In some embodiments, R¹ is 1,3-dihydroxybutan-2-yl. In some embodiments, R¹ is 2-morpholino-2-oxoethyl. In some embodiments, R¹ is 2-(dimethylamino)-2-(pyridin-3-yl)ethyl. In some embodiments, R¹ is 2-(pyrrolidin-1-yl)ethyl. In some embodiments, R¹ is 3-amino-1-methoxy-1-oxopropan-2-yl. In some embodiments, R¹ is 4-amino-1-methoxy-1-oxobutan-2-yl. In some embodiments, R¹ is 1-carboxy-2-hydroxyethyl. In some embodiments, R¹ is (2H-tetrazol-5-yl)methyl. In some embodiments, R¹ is 3-oxo-2,3-dihydroisoxazol-5-yl)methyl. In some embodiments, R¹ is carboxymethyl. In some embodiments, R¹ is 3-carboxypropyl. In some embodiments, R¹ is 2-carboxyethyl. In some embodiments, R¹ is 3-amino-1-carboxy-3-oxopropyl. In some embodiments, R¹ is 1-carboxy-3-methylbutyl. In some embodiments, R¹ is 1,3-dicarboxypropyl. In some embodiments, R¹ is 2-carboxypropan-2-yl. In some embodiments, R¹ is 4-carboxy-1-methoxy-1-oxobutan-2-yl. In some embodiments, R¹ is 3-carboxy-1-methoxy-1-oxopropan-2-yl. In some embodiments, R¹ is 3-(tert-butoxycarbonylamino)-1-carboxypropyl. In some embodiments, R¹ is 2-(tert-butoxycarbonylamino)-1-carboxyethyl. In some embodiments, R¹ is 3-amino-1-carboxypropyl.

In some embodiments, R¹ is 2-amino-1-carboxyethyl. In some embodiments, R¹ is 5-carboxypentyl. In some embodiments, R¹ is 1-amino-1-oxo-3-(phosphonooxy)propan-2-yl. In some embodiments, R¹ is 2-carbamoylcyclopentyl. In some embodiments, R¹ is 2-hydroxycyclopentyl. In some embodiments, R¹ is piperidine-4-carbonyl. In some embodiments, R¹ is 2-aminocyclohexanecarbonyl. In some embodiments, R¹ is morpholine-2-carbonyl. In some embodiments, R¹ is 3-aminopropanoyl. In some embodiments, R¹ is 2-aminoacetyl. In some embodiments, R¹ is 4-hydroxypyrrolidine-2-carbonyl. In some embodiments, R¹ is 2-aminopropanoyl. In some embodiments, R¹ is 2-amino-3-hydroxypropanoyl. In some embodiments, R¹ is 2-hydroxyacetyl. In some embodiments, R¹ is thiomorpholine-3-carbonyl. In some embodiments, R¹ is pyrrolidine-2-carbonyl. In some embodiments, R¹ is 2-(morpholin-4-yl)acetyl. In some embodiments, R¹ is 2-(1H-tetrazol-5-yl)acetyl. In some embodiments, R¹ is 2-(dimethylamino)acetyl. In some embodiments, R¹ is 3-oxo-2,3-dihydroisoxazole-5-carbonyl. In some embodiments, R¹ is 6-oxo-1,6-dihydropyridazine-3-carbonyl. In some embodiments, R¹ is 2,4-dihydroxypyrimidine-5-carbonyl. In some embodiments, R¹ is 5-oxo-4,5-dihydro-1H-1,2,4-triazole-3-carbonyl. In some embodiments, R¹ is 4-aminotetrahydro-2H-thiopyran-4-carbonyl. In some embodiments, R¹ is 2-(3-amino-2-oxopyrrolidin-1-yl)acetyl. In some embodiments, R¹ is 6-hydroxynicotinoyl. In some embodiments, R¹ is 2-hydroxynicotinoyl. In some embodiments, R¹ is 2,6-dihydroxyisonicotinoyl. In some embodiments, R¹ is 2,6-dioxo-1,2,3,6-tetrahydropyrimidine-4-carbonyl. In some embodiments, R¹ is 5-hydroxy-1-methyl-1H-pyrazole-3-carbonyl. In some embodiments, R¹ is 3-(3-hydroxyisoxazol-4-yl)propanoyl. In some embodiments, R¹ is 3-carboxypropanoyl. In some embodiments, R¹ is 5-hydroxypyrazine-2-carbonyl. In some embodiments, R¹ is 6-hydroxypicolinoyl. In some embodiments, R¹ is 4-methylmorpholine-2-carbonyl. In some embodiments, R¹ is 4-ethylmorpholine-2-carbonyl. In some embodiments, R¹ is 4-(2-hydroxyethyl)morpholine-2-carbonyl. In some embodiments, R¹ is 4-(3,3-dimethylbutyl)morpholine-2-carbonyl. In some embodiments, R¹ is 4-(2-hydroxyethyl)morpholine-3-carbonyl. In some embodiments, R¹ is 4-ethylmorpholine-3-carbonyl. In some embodiments, R¹ is 4-(2-hydroxyethyl)thiomorpholine-3-carbonyl. In some embodiments, R¹ is 4-ethylthiomorpholine-3-carbonyl. In some embodiments, R¹ is 3-hydroxypropanoyl. In some embodiments, R¹ is 4-hydroxycyclohexanecarbonyl. In some embodiments, R¹ is 3-hydroxypentanoyl. In some embodiments, R¹ is 2-hydroxy-2-methylpropanoyl. In some embodiments, R¹ is 1-hydroxycyclopropanecarbonyl. In some embodiments, R¹ is 3-hydroxybutanoyl. In some embodiments, R¹ is 3-hydroxy-2,2-dimethylpropanoyl. In some embodiments, R¹ is 4-hydroxybutanoyl. In some embodiments, R¹ is 2-ethyl-2-hydroxybutanoyl. In some embodiments, R¹ is 2-hydroxycyclohexanecarbonyl. In some embodiments, R¹ is 2-cyclohexyl-2-hydroxyacetyl. In some embodiments, R¹ is 3-hydroxy-3-methylbutanoyl. In some embodiments, R¹ is 2-hydroxy-4-methylpentanoyl. In some embodiments, R¹ is 1-(tert-butoxycarbonyl)-4-hydroxypyrrolidine-2-carbonyl. In some embodiments, R¹ is 4-(tert-butoxycarbonyl)thiomorpholine-3-carbonyl. In some embodiments, R¹ is 2-(1-(tert-butoxycarbonyl)piperidin-2-yl)acetyl. In some embodiments, R¹ is 3-hydroxy-2-(hydroxymethyl)-2-methylpropanoyl. In some embodiments, R¹ is 2-(piperidin-2-yl)acetyl. In some embodiments, R¹ is 4-(hydroxymethyl)cyclohexanecarbonyl. In some embodiments, R¹ is 3-(dimethylamino)propanoyl. In some embodiments, R¹ is 2-(pyrrolidin-3-yl)acetyl. In some embodiments, R¹ is 3-(piperidin-1-yl)propanoyl. In some embodiments, R¹ is 4-aminocyclohexanecarbonyl. In some embodiments, R¹ is pyrrolidine-3-carbonyl. In some embodiments, R¹ is 3-(diethylamino)propanoyl. In some embodiments, R¹ is 2-(4-aminocyclohexyl)acetyl. In some embodiments, R¹ is 3-morpholinopropanoyl. In some embodiments, R¹ is 1-methylpiperidine-4-carbonyl. In some embodiments, R¹ is 3-aminocyclohexanecarbonyl. In some embodiments, R¹ is 2-amino-4-carboxybutanoyl. In some embodiments, R¹ is 4-amino-4-carboxybutanoyl. In some embodiments, R¹ is 3-aminocyclopentanecarbonyl. In some embodiments, R¹ is 1-methylpiperidine-3-carbonyl. In some embodiments, R¹ is 2-(piperidin-3-yl)acetyl. In some embodiments, R¹ is azetidine-3-carbonyl. In some embodiments, R¹ is 2-(4-(hydroxymethyl)piperidin-1-yl)acetyl. In some embodiments, R¹ is 2-(3-hydroxypiperidin-1-yl)acetyl. In some embodiments, R¹ is 2-(piperazin-1-yl)acetyl. In some embodiments, R¹ is 2-(3-aminopyrrolidin-1-yl)acetyl. In some embodiments, R¹ is 2-(2-(hydroxymethyl)morpholino)acetyl. In some embodiments, R¹ is 2-(4-propylpiperazin-1-yl)acetyl. In some embodiments, R¹ is 2-(5-oxo-1,4-diazepan-1-yl)acetyl. In some embodiments, R¹ is 2-(4-carbamoylpiperidin-1-yl)acetyl. In some embodiments, R¹ is 2-(2-carbamoylpyrrolidin-1-yl)acetyl. In some embodiments, R¹ is 2-(4-(dimethylamino)piperidin-1-yl)acetyl. In some embodiments, R¹ is 2-(3-(dimethylamino)pyrrolidin-1-yl)acetyl. In some embodiments, R¹ is 2-(4-hydroxypiperidin-1-yl)acetyl. In some embodiments, R¹ is 2-(2,5-diazabicyclo[2.2.1]heptan-2-yl)acetyl. In some embodiments, R¹ is 2-(hexahydropyrrolo[1,2-a]pyrazin-2(1H)-yl)acetyl. In some embodiments, R¹ is 2-(3-(hydroxymethyl)piperidin-1-yl)acetyl. In some embodiments, R¹ is 2-(3-methylpiperazin-1-yl)acetyl. In some embodiments, R¹ is 2-(4-methylpiperidin-1-yl)acetyl. In some embodiments, R¹ is 2-(3-oxopiperazin-1-yl)acetyl. In some embodiments, R¹ is 2-(4-carbamoylpiperazin-1-yl)acetyl. In some embodiments, R¹ is 2-(3-methylpiperidin-1-yl)acetyl. In some embodiments, R¹ is 2-(4-methylpiperazin-1-yl)acetyl. In some embodiments, R¹ is 2-(4-ethylpiperazin-1-yl)acetyl. In some embodiments, R¹ is 2-(2-(2-hydroxyethyl)piperidin-1-yl)acetyl. In some embodiments, R¹ is 2-(3-hydroxypyrrolidin-1-yl)acetyl. In some embodiments, R¹ is 2-(2-(hydroxymethyl)pyrrolidin-1-yl)acetyl. In some embodiments, R¹ is 2-(3-carbamoylpiperidin-1-yl)acetyl. In some embodiments, R¹ is 4-(phosphonooxy)cyclohexanecarbonyl. In some embodiments, R¹ is 2-(phosphonooxy)acetyl. In some embodiments, R¹ is 3-(tert-butoxycarbonylamino)pyrrolidine-1-carbonyl. In some embodiments, R¹ is 2-amino-4-methylpentanoyl. In some embodiments, R¹ is 2-amino-3-cyanopropanoyl. In some embodiments, R¹ is 4-amino-1,1-dioxotetrahydro-2H-thiopyran-4-carbonyl. In some embodiments, R¹ is 2,4-diamino-4-oxobutanoyl. In some embodiments, R¹ is 3-amino-2-hydroxypropanoyl. In some embodiments, R¹ is 2-hydroxypropanoyl. In some embodiments, R¹ is 5-(hydroxymethyl)-1H-1,2,3-triazole-4-carbonyl. In some embodiments, R¹ is piperazine-1-carbonyl. In some embodiments, R¹ is 4-ethylpiperazine-1-carbonyl. In some embodiments, R¹ is 1,1-dioxotetrahydro-2H-thiopyran-4-carbonyl. In some embodiments, R¹ is 3-hydroxypyrrolidine-1-carbonyl. In some embodiments, R¹ is 4-(2-hydroxyethyl)piperazine-1-carbonyl. In some embodiments, R¹ is 4-(hydroxymethyl)piperidine-1-carbonyl. In some embodiments, R¹ is 3-aminopiperidine-1-carbonyl. In some embodiments, R¹ is 3-hydroxyazetidine-1-carbonyl. In some embodiments, R¹ is 3-aminopyrrolidine-1-carbonyl. In some embodiments, R¹ is 2-carbamoylpyrrolidine-1-carbonyl. In some embodiments, R¹ is 4-(dimethylamino)piperidine-1-carbonyl. In some embodiments, R¹ is 4-carbamoylpiperazine-1-carbonyl. In some embodiments, R¹ is 3-oxopiperazine-1-carbonyl. In some embodiments, R¹ is 2-(hydroxymethyl)pyrrolidine-1-carbonyl. In some embodiments, R¹ is 2-(2-hydroxyethyl)piperidine-1-carbonyl. In some embodiments, R¹ is 2-(hydroxymethyl)morpholine-4-carbonyl. In some embodiments, R¹ is 3-carboxyazetidine-1-carbonyl. In some embodiments, R¹ is 4-(3-hydroxypropyl)piperidine-1-carbonyl. In some embodiments, R¹ is 3-hydroxypiperidine-1-carbonyl. In some embodiments, R¹ is 4-cyanopiperidine-1-carbonyl. In some embodiments, R¹ is 2-(hydroxymethyl)piperidine-1-carbonyl. In some embodiments, R¹ is 4-hydroxypiperidine-1-carbonyl. In some embodiments, R¹ is 2-oxopyrrolidine-1-carbonyl. In some embodiments, R¹ is 3-(hydroxymethyl)piperidine-1-carbonyl. In some embodiments, R¹ is 3-(hydroxymethyl)pyrrolidine-1-carbonyl. In some embodiments, R¹ is 3-(phosphonooxy)pyrrolidine-1-carbonyl. In some embodiments, R¹ is 1-(tert-butoxycarbonyl)piperidine-4-carbonyl. In some embodiments, R¹ is 2-(tert-butoxycarbonylamino)-cyclohexanecarbonyl. In some embodiments, R¹ is 1-(tert-butoxycarbonyl)piperidine-3-carbonyl. In some embodiments, R¹ is 3-(tert-butoxycarbonylamino)piperidine-1-carbonyl. In some embodiments, R¹ is 4-(tert-butoxycarbonyl)morpholine-2-carbonyl. In some embodiments, R¹ is 3-(tert-butoxycarbonylamino)propanoyl. In some embodiments, R¹ is 2-(tert-butoxycarbonylamino)acetyl. In some embodiments, R¹ is 3-(tert-butoxycarbonylamino)-2-hydroxypropanoyl. In some embodiments, R¹ is 2-(tert-butoxycarbonylamino)propanoyl. In some embodiments, R¹ is 2-(tert-butoxycarbonylamino)-3-hydroxypropanoyl. In some embodiments, R¹ is 1-(tert-butoxycarbonyl)pyrrolidine-2-carbonyl. In some embodiments, R¹ is 4-(tert-butoxycarbonylamino)tetrahydro-2H-thiopyran-4-carbonyl. In some embodiments, R¹ is 4-tert-butoxy-4-oxobutanoyl. In some embodiments, R¹ is 2-(3-(tert-butoxycarbonylamino)-2-oxopyrrolidin-1-yl)acetyl. In some embodiments, R¹ is 4-amino-2-(tert-butoxycarbonylamino)-4-oxobutanoyl. In some embodiments, R¹ is 2-(1-(tert-butoxycarbonyl)pyrrolidin-3-yl)acetyl. In some embodiments, R¹ is 4-(tert-butoxycarbonylamino)cyclohexanecarbonyl. In some embodiments, R¹ is 1-(tert-butoxycarbonyl)pyrrolidine-3-carbonyl. In some embodiments, R¹ is 2-(4-(tert-butoxycarbonylamino)cyclohexyl)acetyl. In some embodiments, R¹ is 3-(tert-butoxycarbonylamino)cyclohexanecarbonyl. In some embodiments, R¹ is 2-(tert-butoxycarbonylamino)-4-carboxybutanoyl. In some embodiments, R¹ is 4-(tert-butoxycarbonylamino)-4-carboxybutanoyl. In some embodiments, R¹ is 3-(tert-butoxycarbonylamino)cyclopentanecarbonyl. In some embodiments, R¹ is 2-(1-(tert-butoxycarbonyl)piperidin-3-yl)acetyl. In some embodiments, R¹ is 1-(tert-butoxycarbonyl)azetidine-3-carbonyl. In some embodiments, R¹ is 2-(3-(tert-butoxycarbonylamino)pyrrolidin-1-yl. In some embodiments, R¹ is 2-(4-(tert-butoxycarbonyl)-3-methylpiperazin-1-yl)acetyl. In some embodiments, R¹ is 2-(tert-butoxycarbonylamino)-4-methylpentanoyl. In some embodiments, R¹ is 2-(tert-butoxycarbonylamino)-3-cyanopropanoyl. In some embodiments, R¹ is 4-(tert-butoxycarbonylamino)-1,1-dioxotetrahydro-2H-thiopyran-4-carbonyl.

In some embodiments, R¹ is butyl. In some embodiments, R¹ is 3-hydroxypropyl. In some embodiments, R¹ is 3,3-dimethylbutyl. In some embodiments, R¹ is (tetrahydro-2H-pyran-4-yl)methyl. In some embodiments, R¹ is 2-methoxyethyl. In some embodiments, R¹ is 3-amino-3-oxopropyl. In some embodiments, R¹ is 2-ethoxy-2-oxoethyl. In some embodiments, R¹ is 2-amino-2-oxoethyl. In some embodiments, R¹ is 2-ethoxyethyl. In some embodiments, R¹ is 2-(methylsulfonyl)ethyl. In some embodiments, R¹ is a group other than H.

### the Group R²

In some embodiments, R² is selected from: H and C₁-C₆ alkyl, wherein said C₁-C₆ alkyl is optionally substituted with one or more substituents selected from: hydroxyl and cyano.

In some embodiments, R² is selected from: H, ethyl, methyl, propan-2-yl, and tert-butyl, each optionally substituted with one or more substituents selected from: cyano and hydroxyl.

In some embodiments, R² is selected from: H, ethyl, methyl, isopropyl, 2-hydroxyethyl, 2-cyanoethyl, and tert-butyl.

In some embodiments, R² is H. In some embodiments, R² is ethyl. In some embodiments, R² is methyl. In some embodiments, R² is isopropyl. In some embodiments, R² is 2-hydroxyethyl. In some embodiments, R² is 2-cyanoethyl. In some embodiments, R² is tert-butyl.

### the Group R³

In some embodiments, R³ is selected from: H and halogen. In some embodiments, R³ is selected from: H, fluoro, and chloro. In some embodiments, R³ is selected from: H and chloro. In some embodiments, R³ is H. In some embodiments, R³ is chloro.

### the Groups R¹ and R²

In some embodiments, R¹ and R² together with the nitrogen atom to which they are both bonded form a group selected from: heteroaryl and heterocyclyl, each optionally substituted with one or more substituents selected from: C₁-C₆ alkoxycarbonyl, C₁-C₆ alkoxycarbonylamino, C₁-C₆ alkyl, C₁-C₆ alkylcarboxamide, C₁-C₆ alkylsulfonyl, amino, C₃-C₇ cycloalkyl, C₄-C₁₃ cycloalkylalkyl, carboxamide, carboxyl, C₂-C₆ dialkylamino, C₂-C₆ dialkylcarboxamide, heteroaryl-C₁-C₆-alkyl, heterocyclyl, heterocyclyl-C₁-C₆-alkyl, hydroxyl, hydroxyheterocyclyl, and oxo, wherein said C₁-C₆ alkyl and C₁-C₆ alkylcarboxamide are each optionally substituted with one or more substituents selected from: carboxyl, hydroxyl, and oxo.

In some embodiments, R¹ and R² together with the nitrogen atom to which they are both bonded form a group selected from: heteroaryl and heterocyclyl, each optionally substituted with one or more substituents selected from: methoxycarbonyl, ethoxycarbonyl, tert-butoxycarbonyl, ethyl, methyl, methylcarbamoyl, ethylcarbamoyl, isopropylcarbamoyl, methylsulfonyl, amino, cyclopentyl, 2-cyclohexylethyl, cyclohexylmethyl, carboxamide, carboxyl, dimethylamino, diethylcarbamoyl, 2-(pyridin-2-yl)ethyl, morpholino, piperidin-1-yl, pyrrolidin-1-ylmethyl, hydroxyl, 4-hydroxy-piperidin-1-yl, and oxo, wherein said ethyl, methyl, ethylcarbamoyl, isopropylcarbamoyl are each optionally substituted with one or more substituents selected from: carboxyl, hydroxyl, and oxo.

In some embodiments, R¹ and R² together with the nitrogen atom to which they are both bonded form a group selected from: 1H-imidazol-1-yl, 1H-1,2,4-triazol-1-yl, 1H-pyrazol-1-yl, 1H-pyrrol-1-yl, 2H-tetrazol-5-yl, 6,7-dihydro-1H-imidazo[4,5-c]pyridin-5(4H)-yl, 5,6-dihydroimidazo[1,2-a]pyrazin-7(8H)-yl, thiomorpholin-4-yl, pyrrolidin-1-yl, piperazin-1-yl, hexahydropyrrolo[1,2-a]pyrazin-2(1H)-yl, piperidin-1-yl, morpholino, 2,7-diazaspiro[4.4]nonan-2-yl, 5,6-dihydropyrimidin-1(4H)-yl, 2,3,4,6,7,8-hexahydro-1H-pyrimido[1,2-a]pyrimidin-1-yl, 1,4-oxazepan-4-yl, azetidin-1-yl, 2,5-diazabicyclo[2.2.1]heptan-2-yl, 1,4-diazepan-1-yl, 2,7-diazaspiro[3.5]nonan-2-yl, imidazolidin-1-yl, tetrahydropyrimidin-1(2H)-yl, each optionally substituted with one or more substituents selected from: C₁-C₆ alkoxycarbonyl, C₁-C₆ alkoxycarbonylamino, C₁-C₆ alkyl, C₁-C₆ alkylcarboxamide, C₁-C₆ alkylsulfonyl, amino, C₃-C₇ cycloalkyl, C₄-C₁₃ cycloalkylalkyl, carboxamide, carboxyl, C₂-C₆ dialkylamino, C₂-C₆ dialkylcarboxamide, heteroaryl-C₁-C₆-alkyl, heterocyclyl, heterocyclyl-C₁-C₆-alkyl, hydroxyl, hydroxyheterocyclyl, and oxo, wherein said C₁-C₆ alkyl and C₁-C₆ alkylcarboxamide are each optionally substituted with one or more substituents selected from: carboxyl, hydroxyl, and oxo.

In some embodiments, R¹ and R² together with the nitrogen atom to which they are both bonded form a group selected from: 1H-imidazol-1-yl, 1H-1,2,4-triazol-1-yl, 1H-pyrazol-1-yl, 1H-pyrrol-1-yl, 2H-tetrazol-5-yl, 6,7-dihydro-1H-imidazo[4,5-c]pyridin-5(4H)-yl, 5,6-dihydroimidazo[1,2-a]pyrazin-7(8H)-yl, thiomorpholin-4-yl, pyrrolidin-1-yl, piperazin-1-yl, hexahydropyrrolo[1,2-a]pyrazin-2(1H)-yl, piperidin-1-yl, morpholino, 2,7-diazaspiro[4.4]nonan-2-yl, 5,6-dihydropyrimidin-1(4H)-yl, 2,3,4,6,7,8-hexahydro-1H-pyrimido[1,2-a]pyrimidin-1-yl, 1,4-oxazepan-4-yl, azetidin-1-yl, 2,5-diazabicyclo[2.2.1]heptan-2-yl, 1,4-diazepan-1-yl, and 2,7-diazaspiro[3.5]nonan-2-yl, each optionally substituted with one or more substituents selected from: C₁-C₆ alkoxycarbonyl, C₁-C₆ alkoxycarbonylamino, C₁-C₆ alkyl, C₁-C₆ alkylcarboxamide, C₁-C₆ alkylsulfonyl, amino, C₃-C₇ cycloalkyl, C₄-C₁₃ cycloalkylalkyl, carboxamide, carboxyl, C₂-C₆ dialkylamino, C₂-C₆ dialkylcarboxamide, heteroaryl-C₁-C₆-alkyl, heterocyclyl, heterocyclyl-C₁-C₆-alkyl, hydroxyl, hydroxyheterocyclyl, and oxo, wherein said C₁-C₆ alkyl and C₁-C₆ alkylcarboxamide are each optionally substituted with one or more substituents selected from: carboxyl, hydroxyl, and oxo.

In some embodiments, R¹ and R² together with the nitrogen atom to which they are both bonded form a group selected from: 1H-imidazol-1-yl, 1H-1,2,4-triazol-1-yl, 1H-pyrazol-1-yl, 1H-pyrrol-1-yl, 2H-tetrazol-5-yl, 6,7-dihydro-1H-imidazo[4,5-c]pyridin-5(4H)-yl, 5,6-dihydroimidazo[1,2-a]pyrazin-7(8H)-yl, thiomorpholin-4-yl, pyrrolidin-1-yl, piperazin-1-yl, hexahydropyrrolo[1,2-a]pyrazin-2(1H)-yl, piperidin-1-yl, morpholino, 2,7-diazaspiro[4.4]nonan-2-yl, 5,6-dihydropyrimidin-1(4H)-yl, 2,3,4,6,7,8-hexahydro-1H-pyrimido[1,2-a]pyrimidin-1-yl, 1,4-oxazepan-4-yl, azetidin-1-yl, 2,5-diazabicyclo[2.2.1]heptan-2-yl, 1,4-diazepan-1-yl, 2,7-diazaspiro[3.5]nonan-2-yl, imidazolidin-1-yl, and tetrahydropyrimidin-1(2H)-yl, each optionally substituted with one or more substituents selected from: methoxycarbonyl, ethoxycarbonyl, tert-butoxycarbonyl, ethyl, methyl, isobutyl, methylcarbamoyl, ethylcarbamoyl, isopropylcarbamoyl, methylsulfonyl, amino, cyclopentyl, 2-cyclohexylethyl, cyclohexylmethyl, carboxamide, carboxyl, dimethylamino, diethylcarbamoyl, 2-(pyridin-2-yl)ethyl, morpholino, piperidin-1-yl, pyrrolidin-1-ylmethyl, hydroxyl, 4-hydroxy-piperidin-1-yl, and oxo, wherein said ethyl, methyl, ethylcarbamoyl, isopropylcarbamoyl are each optionally substituted with one or more substituents selected from: carboxyl, hydroxyl, and oxo.

In some embodiments, R¹ and R² together with the nitrogen atom to which they are both bonded form a group selected from: 1H-imidazol-1-yl, 1H-1,2,4-triazol-1-yl, 1H-pyrazol-1-yl, 1H-pyrrol-1-yl, 2H-tetrazol-5-yl, 6,7-dihydro-1H-imidazo[4,5-c]pyridin-5(4H)-yl, 5,6-dihydroimidazo[1,2-a]pyrazin-7(8H)-yl, thiomorpholin-4-yl, pyrrolidin-1-yl, piperazin-1-yl, hexahydropyrrolo[1,2-a]pyrazin-2(1H)-yl, piperidin-1-yl, morpholino, 2,7-diazaspiro[4.4]nonan-2-yl, 5,6-dihydropyrimidin-1(4H)-yl, 2,3,4,6,7,8-hexahydro-1H-pyrimido[1,2-a]pyrimidin-1-yl, 1,4-oxazepan-4-yl, azetidin-1-yl, 2,5-diazabicyclo[2.2.1]heptan-2-yl, 1,4-diazepan-1-yl, and 2,7-diazaspiro[3.5]nonan-2-yl, each optionally substituted with one or more substituents selected from: methoxycarbonyl, ethoxycarbonyl, tert-butoxycarbonyl, ethyl, methyl, methylcarbamoyl, ethylcarbamoyl, isopropylcarbamoyl, methylsulfonyl, amino, cyclopentyl, 2-cyclohexylethyl, cyclohexylmethyl, carboxamide, carboxyl, dimethylamino, diethylcarbamoyl, 2-(pyridin-2-yl)ethyl, morpholino, piperidin-1-yl, pyrrolidin-1-ylmethyl, hydroxyl, 4-hydroxy-piperidin-1-yl, and oxo, wherein said ethyl, methyl, ethylcarbamoyl, isopropylcarbamoyl are each optionally substituted with one or more substituents selected from: carboxyl, hydroxyl, and oxo.

In some embodiments, R¹ and R² together with the nitrogen atom to which they are both bonded form a group selected from: 1,1-dioxo-thiomorpholin-4-yl, 3-hydroxypyrrolidin-1-yl, 4-(2-hydroxyethyl)piperazin-1-yl, hexahydropyrrolo[1,2-a]pyrazin-2(1H)-yl, 4-ethylpiperazin-1-yl, piperidin-1-yl, 1H-imidazol-1-yl, morpholino, 4-methylpiperazin-1-yl, pyrrolidin-1-yl, 1H-1,2,4-triazol-1-yl, 1H-pyrazol-1-yl, 1H-pyrrol-1-yl, 2H-tetrazol-5-yl, piperazin-1-yl, 4-(dimethylamino)piperidin-1-yl, 4-(hydroxymethyl)piperidin-1-yl, 2-carbamoylpyrrolidin-1-yl, 2-(2-hydroxyethyl)piperidin-1-yl, 4-carbamoylpiperazin-1-yl, 3-oxopiperazin-1-yl, 4-(2-cyclohexylethyl)piperazin-1-yl, 2,7-diazaspiro[4.4]nonan-2-yl, 3-(methylsulfonyl)pyrrolidin-1-yl, 6,7-dihydro-1H-imidazo[4,5-c]pyridin-5(4H)-yl, 2-(hydroxymethyl)piperidin-1-yl, 3-aminopyrrolidin-1-yl, 2-methylpiperazin-1-yl, 3-aminopiperidin-1-yl, 4-aminopiperidin-1-yl, 2-carbamoylpiperidin-1-yl, 5,6-dihydropyrimidin-1(4H)-yl, 4-hydroxy-2-(methoxycarbonyl)pyrrolidin-1-yl, 4-hydroxypiperidin-1-yl, 4-(2-(pyridin-2-yl)ethyl)piperazin-1-yl, 3-hydroxypiperidin-1-yl, 3-(diethylcarbamoyl)piperidin-1-yl, 2,3,4,6,7,8-hexahydro-1H-pyrimido[1,2-a]pyrimidin-1-yl, 4-cyclopentylpiperazin-1-yl, 1,4-oxazepan-4-yl, 2-(pyrrolidin-1-ylmethyl)pyrrolidin-1-yl, 4-morpholinopiperidin-1-yl, 4-(cyclohexylmethyl)piperazin-1-yl, 4-oxopiperidin-1-yl, 4-acetylpiperazin-1-yl, 1,4′-bipiperidin-1′-yl, 4-(ethoxycarbonyl)piperidin-1-yl, 2-(hydroxymethyl)morpholino, 2-(hydroxymethyl)pyrrolidin-1-yl, 3-hydroxyazetidin-1-yl, 4-hydroxy-1,4′-bipiperidin-1′-yl, 3-(hydroxymethyl)piperidin-1-yl, 2,5-diazabicyclo[2.2.1]heptan-2-yl, 5-oxo-1,4-diazepan-1-yl, 4-(2-hydroxyethyl)piperidin-1-yl, 3-(carboxymethyl)pyrrolidin-1-yl, 2,7-diazaspiro[3.5]nonan-2-yl, 4-(tert-butoxycarbonyl)-2-(carboxymethyl)piperazin-1-yl, 4-(tert-butoxycarbonyl)-2-carboxypiperazin-1-yl, 4-carboxypiperidin-1-yl, 2-(carboxymethyl)morpholino, 2-(carboxymethyl)piperazin-1-yl, 2-carboxypiperazin-1-yl, 4-(carboxymethyl)piperazin-1-yl, 2-carboxy-5,6-dihydroimidazo[1,2-a]pyrazin-7(8H)-yl, 2-carbamoylpiperazin-1-yl, 2-(methylcarbamoyl)piperazin-1-yl, 2-(2-hydroxyethylcarbamoyl)piperazin-1-yl, 2-(1-hydroxypropan-2-ylcarbamoyl)piperazin-1-yl, 3-carbamoylpiperidin-1-yl, 4-carbamoylpiperidin-1-yl, 3-(hydroxymethyl)pyrrolidin-1-yl, 2-oxopyrrolidin-1-yl, 2,5-dioxoimidazolidin-1-yl, 2,6-dioxotetrahydropyrimidin-1(2H)-yl, 3-methyl-2,5-dioxoimidazolidin-1-yl, and 4-isobutyl-2,5-dioxoimidazolidin-1-yl.

In some embodiments, R¹ and R² together with the nitrogen atom to which they are both bonded form a group selected from: 1,1-dioxo-thiomorpholin-4-yl, 3-hydroxypyrrolidin-1-yl, 4-(2-hydroxyethyl)piperazin-1-yl, hexahydropyrrolo[1,2-a]pyrazin-2(1H)-yl, 4-ethylpiperazin-1-yl, piperidin-1-yl, 1H-imidazol-1-yl, morpholino, 4-methylpiperazin-1-yl, pyrrolidin-1-yl, 1H-1,2,4-triazol-1-yl, 1H-pyrazol-1-yl, 1H-pyrrol-1-yl, 2H-tetrazol-5-yl, piperazin-1-yl, 4-(dimethylamino)piperidin-1-yl, 4-(hydroxymethyl)piperidin-1-yl, 2-carbamoylpyrrolidin-1-yl, 2-(2-hydroxyethyl)piperidin-1-yl, 4-carbamoylpiperazin-1-yl, 3-oxopiperazin-1-yl, 4-(2-cyclohexylethyl)piperazin-1-yl, 2,7-diazaspiro[4.4]nonan-2-yl, 3-(methylsulfonyl)pyrrolidin-1-yl, 6,7-dihydro-1H-imidazo[4,5-c]pyridin-5(4H)-yl, 2-(hydroxymethyl)piperidin-1-yl, 3-aminopyrrolidin-1-yl, 2-methylpiperazin-1-yl, 3-aminopiperidin-1-yl, 4-aminopiperidin-1-yl, 2-carbamoylpiperidin-1-yl, 5,6-dihydropyrimidin-1(4H)-yl, 4-hydroxy-2-(methoxycarbonyl)pyrrolidin-1-yl, 4-hydroxypiperidin-1-yl, 4-(2-(pyridin-2-yl)ethyl)piperazin-1-yl, 3-hydroxypiperidin-1-yl, 3-(diethylcarbamoyl)piperidin-1-yl, 2,3,4,6,7,8-hexahydro-1H-pyrimido[1,2-a]pyrimidin-1-yl, 4-cyclopentylpiperazin-1-yl, 1,4-oxazepan-4-yl, 2-(pyrrolidin-1-ylmethyl)pyrrolidin-1-yl, 4-morpholinopiperidin-1-yl, 4-(cyclohexylmethyl)piperazin-1-yl, 4-oxopiperidin-1-yl, 4-acetylpiperazin-1-yl, 1,4′-bipiperidin-1′-yl, 4-(ethoxycarbonyl)piperidin-1-yl, 2-(hydroxymethyl)morpholino, 2-(hydroxymethyl)pyrrolidin-1-yl, 3-hydroxyazetidin-1-yl, 4-hydroxy-1,4′-bipiperidin-1′-yl, 3-(hydroxymethyl)piperidin-1-yl, 2,5-diazabicyclo[2.2.1]heptan-2-yl, 5-oxo-1,4-diazepan-1-yl, 4-(2-hydroxyethyl)piperidin-1-yl, 3-(carboxymethyl)pyrrolidin-1-yl, 2,7-diazaspiro[3.5]nonan-2-yl, 4-(tert-butoxycarbonyl)-2-(carboxymethyl)piperazin-1-yl, 4-(tert-butoxycarbonyl)-2-carboxypiperazin-1-yl, 4-carboxypiperidin-1-yl, 2-(carboxymethyl)morpholino, 2-(carboxymethyl)piperazin-1-yl, 2-carboxypiperazin-1-yl, 4-(carboxymethyl)piperazin-1-yl, 2-carboxy-5,6-dihydroimidazo[1,2-a]pyrazin-7(8H)-yl, 2-carbamoylpiperazin-1-yl, 2-(methylcarbamoyl)piperazin-1-yl, 2-(2-hydroxyethylcarbamoyl)piperazin-1-yl, 2-(1-hydroxypropan-2-ylcarbamoyl)piperazin-1-yl, 3-carbamoylpiperidin-1-yl, 4-carbamoylpiperidin-1-yl, and 3-(hydroxymethyl)pyrrolidin-1-yl.

In some embodiments, R¹ and R² together with the nitrogen atom to which they are both bonded form 1,1-dioxo-thiomorpholin-4-yl. In some embodiments, R¹ and R² together with the nitrogen atom to which they are both bonded form 3-hydroxypyrrolidin-1-yl. In some embodiments, R¹ and R² together with the nitrogen atom to which they are both bonded form 4-(2-hydroxyethyl)piperazin-1-yl. In some embodiments, R¹ and R² together with the nitrogen atom to which they are both bonded form hexahydropyrrolo[1,2-a]pyrazin-2(1H)-yl. In some embodiments, R¹ and R² together with the nitrogen atom to which they are both bonded form 4-ethylpiperazin-1-yl. In some embodiments, R¹ and R² together with the nitrogen atom to which they are both bonded form piperidin-1-yl. In some embodiments, R¹ and R² together with the nitrogen atom to which they are both bonded form 1H-imidazol-1-yl. In some embodiments, R¹ and R² together with the nitrogen atom to which they are both bonded form morpholino. In some embodiments, R¹ and R² together with the nitrogen atom to which they are both bonded form 4-methylpiperazin-1-yl. In some embodiments, R¹ and R² together with the nitrogen atom to which they are both bonded form pyrrolidin-1-yl. In some embodiments, R¹ and R² together with the nitrogen atom to which they are both bonded form 1H-1,2,4-triazol-1-yl. In some embodiments, R¹ and R² together with the nitrogen atom to which they are both bonded form 1H-pyrazol-1-yl. In some embodiments, R¹ and R² together with the nitrogen atom to which they are both bonded form 1H-pyrrol-1-yl. In some embodiments, R¹ and R² together with the nitrogen atom to which they are both bonded form 2H-tetrazol-5-yl. In some embodiments, R¹ and R² together with the nitrogen atom to which they are both bonded form piperazin-1-yl. In some embodiments, R¹ and R² together with the nitrogen atom to which they are both bonded form 4-(dimethylamino)piperidin-1-yl. In some embodiments, R¹ and R² together with the nitrogen atom to which they are both bonded form 4-(hydroxymethyl)piperidin-1-yl. In some embodiments, R¹ and R² together with the nitrogen atom to which they are both bonded form 2-carbamoylpyrrolidin-1-yl. In some embodiments, R¹ and R² together with the nitrogen atom to which they are both bonded form 2-(2-hydroxyethyl)piperidin-1-yl. In some embodiments, R¹ and R² together with the nitrogen atom to which they are both bonded form 4-carbamoylpiperazin-1-yl. In some embodiments, R¹ and R² together with the nitrogen atom to which they are both bonded form 3-oxopiperazin-1-yl. In some embodiments, R¹ and R² together with the nitrogen atom to which they are both bonded form 4-(2-cyclohexylethyl)piperazin-1-yl. In some embodiments, R¹ and R² together with the nitrogen atom to which they are both bonded form 2,7-diazaspiro[4.4]nonan-2-yl. In some embodiments, R¹ and R² together with the nitrogen atom to which they are both bonded form 3-(methylsulfonyl)pyrrolidin-1-yl. In some embodiments, R¹ and R² together with the nitrogen atom to which they are both bonded form 6,7-dihydro-1H-imidazo[4,5-c]pyridin-5(4H)-yl. In some embodiments, R¹ and R² together with the nitrogen atom to which they are both bonded form 2-(hydroxymethyl)piperidin-1-yl. In some embodiments, R¹ and R² together with the nitrogen atom to which they are both bonded form 3-aminopyrrolidin-1-yl. In some embodiments, R¹ and R² together with the nitrogen atom to which they are both bonded form 2-methylpiperazin-1-yl. In some embodiments, R¹ and R² together with the nitrogen atom to which they are both bonded form 3-aminopiperidin-1-yl. In some embodiments, R¹ and R² together with the nitrogen atom to which they are both bonded form 4-aminopiperidin-1-yl. In some embodiments, R¹ and R² together with the nitrogen atom to which they are both bonded form 2-carbamoylpiperidin-1-yl. In some embodiments, R¹ and R² together with the nitrogen atom to which they are both bonded form 5,6-dihydropyrimidin-1(4H)-yl. In some embodiments, R¹ and R² together with the nitrogen atom to which they are both bonded form 4-hydroxy-2-(methoxycarbonyl)pyrrolidin-1-yl. In some embodiments, R¹ and R² together with the nitrogen atom to which they are both bonded form 4-hydroxypiperidin-1-yl. In some embodiments, R¹ and R² together with the nitrogen atom to which they are both bonded form 2-(pyridin-2-yl)ethyl)piperazin-1-yl. In some embodiments, R¹ and R² together with the nitrogen atom to which they are both bonded form 3-hydroxypiperidin-1-yl. In some embodiments, R¹ and R² together with the nitrogen atom to which they are both bonded form 3-(diethylcarbamoyl)piperidin-1-yl. In some embodiments, R¹ and R² together with the nitrogen atom to which they are both bonded form 2,3,4,6,7,8-hexahydro-1H-pyrimido[1,2-a]pyrimidin-1-yl. In some embodiments, R¹ and R² together with the nitrogen atom to which they are both bonded form 4-cyclopentylpiperazin-1-yl. In some embodiments, R¹ and R² together with the nitrogen atom to which they are both bonded form 1,4-oxazepan-4-yl. In some embodiments, R¹ and R² together with the nitrogen atom to which they are both bonded form 2-(pyrrolidin-1-ylmethyl)pyrrolidin-1-yl. In some embodiments, R¹ and R² together with the nitrogen atom to which they are both bonded form 4-morpholinopiperidin-1-yl. In some embodiments, R¹ and R² together with the nitrogen atom to which they are both bonded form 4-(cyclohexylmethyl)piperazin-1-yl. In some embodiments, R¹ and R² together with the nitrogen atom to which they are both bonded form 4-oxopiperidin-1-yl. In some embodiments, R¹ and R² together with the nitrogen atom to which they are both bonded form 4-acetylpiperazin-1-yl. In some embodiments, R¹ and R² together with the nitrogen atom to which they are both bonded form 1,4′-bipiperidin-1′-yl. In some embodiments, R¹ and R² together with the nitrogen atom to which they are both bonded form 4-(ethoxycarbonyl)piperidin-1-yl. In some embodiments, R¹ and R² together with the nitrogen atom to which they are both bonded form 2-(hydroxymethyl)morpholino. In some embodiments, R¹ and R² together with the nitrogen atom to which they are both bonded form 2-(hydroxymethyl)pyrrolidin-1-yl. In some embodiments, R¹ and R² together with the nitrogen atom to which they are both bonded form 3-hydroxyazetidin-1-yl. In some embodiments, R¹ and R² together with the nitrogen atom to which they are both bonded form 4-hydroxy-1,4′-bipiperidin-1′-yl. In some embodiments, R¹ and R² together with the nitrogen atom to which they are both bonded form 3-(hydroxymethyl)piperidin-1-yl. In some embodiments, R¹ and R² together with the nitrogen atom to which they are both bonded form 2,5-diazabicyclo[2.2.1]heptan-2-yl. In some embodiments, R¹ and R² together with the nitrogen atom to which they are both bonded form 5-oxo-1,4-diazepan-1-yl. In some embodiments, R¹ and R² together with the nitrogen atom to which they are both bonded form 4-(2-hydroxyethyl)piperidin-1-yl. In some embodiments, R¹ and R² together with the nitrogen atom to which they are both bonded form 3-(carboxymethyl)pyrrolidin-1-yl. In some embodiments, R¹ and R² together with the nitrogen atom to which they are both bonded form 2,7-diazaspiro[3.5]nonan-2-yl. In some embodiments, R¹ and R² together with the nitrogen atom to which they are both bonded form 4-(tert-butoxycarbonyl)-2-(carboxymethyl)piperazin-1-yl. In some embodiments, R¹ and R² together with the nitrogen atom to which they are both bonded form 4-(tert-butoxycarbonyl)-2-carboxypiperazin-1-yl. In some embodiments, R¹ and R² together with the nitrogen atom to which they are both bonded form 4-carboxypiperidin-1-yl. In some embodiments, R¹ and R² together with the nitrogen atom to which they are both bonded form 2-(carboxymethyl)morpholino. In some embodiments, R¹ and R² together with the nitrogen atom to which they are both bonded form 2-(carboxymethyl)piperazin-1-yl. In some embodiments, R¹ and R² together with the nitrogen atom to which they are both bonded form 2-carboxypiperazin-1-yl. In some embodiments, R¹ and R² together with the nitrogen atom to which they are both bonded form 4-(carboxymethyl)piperazin-1-yl. In some embodiments, R¹ and R² together with the nitrogen atom to which they are both bonded form 2-carboxy-5,6-dihydroimidazo[1,2-a]pyrazin-7(8H)-yl. In some embodiments, R¹ and R² together with the nitrogen atom to which they are both bonded form 2-carbamoylpiperazin-1-yl. In some embodiments, R¹ and R² together with the nitrogen atom to which they are both bonded form 2-(methylcarbamoyl)piperazin-1-yl. In some embodiments, R¹ and R² together with the nitrogen atom to which they are both bonded form 2-(2-hydroxyethylcarbamoyl)piperazin-1-yl. In some embodiments, R¹ and R² together with the nitrogen atom to which they are both bonded form 2-(1-hydroxypropan-2-ylcarbamoyl)piperazin-1-yl. In some embodiments, R¹ and R² together with the nitrogen atom to which they are both bonded form 3-carbamoylpiperidin-1-yl. In some embodiments, R¹ and R² together with the nitrogen atom to which they are both bonded form 4-carbamoylpiperidin-1-yl. In some embodiments, R¹ and R² together with the nitrogen atom to which they are both bonded form 3-(hydroxymethyl)pyrrolidin-1-yl. In some embodiments, R¹ and R² together with the nitrogen atom to which they are both bonded form 2-oxopyrrolidin-1-yl. In some embodiments, R¹ and R² together with the nitrogen atom to which they are both bonded form 2,5-dioxoimidazolidin-1-yl. In some embodiments, R¹ and R² together with the nitrogen atom to which they are both bonded form 2,6-dioxotetrahydropyrimidin-1(2H)-yl. In some embodiments, R¹ and R² together with the nitrogen atom to which they are both bonded form 3-methyl-2,5-dioxoimidazolidin-1-yl. In some embodiments, R¹ and R² together with the nitrogen atom to which they are both bonded form 4-isobutyl-2,5-dioxoimidazolidin-1-yl.

### the Groups R² and R³

In some embodiments, R² and R³ together form CH₂.

In some embodiments, R² and R³ together with the nitrogen atom to which R² is bonded, and the phenyl ring to which R³ is bonded, and X form a group selected from: 1,2,3,4-tetrahydroisoquinolinyl and isoindolinyl.

### the Groups R⁴, R⁵, R⁶, and R⁷

In some embodiments, R⁴, R⁵, R⁶, and R⁷ are each selected independently from: H and halogen.

In some embodiments, R⁴, R⁵, R⁶, and R⁷ are independently selected from: H, bromo, fluoro, and chloro.

In some embodiments, R⁴, R⁵, R⁶, and R⁷ are independently selected from: H, fluoro, and chloro.

### the Groups R⁴ and R⁵

In some embodiments, R⁴ and R⁵ are each selected independently from: H and halogen.

In some embodiments, R⁴ and R⁵ are each selected independently from: H and fluoro.

In some embodiments, R⁴ and R⁵ are each fluoro.

### the Groups R⁶ and R⁷

In some embodiments, R⁶ and R⁷ are each selected independently from: H and halogen.

In some embodiments, R⁶ is H; and R⁷ is fluoro.

### the Group R⁴

In some embodiments, R⁴ is selected from: H and halogen.

In some embodiments, R⁴ is selected from: H, fluoro, and chloro.

In some embodiments, R⁴ is selected from: fluoro and chloro.

In some embodiments, R⁴ is H.

In some embodiments, R⁴ is fluoro.

In some embodiments, R⁴ is chloro.

### the Group R⁵

In some embodiments, R⁵ is selected from: H and halogen.

In some embodiments, R⁵ is selected from: H and fluoro.

In some embodiments, R⁵ is H.

In some embodiments, R⁵ is fluoro.

### the Group R⁶

In some embodiments, R⁶ is selected from: H and halogen.

In some embodiments, R⁶ is selected from: H, fluoro, and chloro.

In some embodiments, R⁶ is selected from: H and chloro.

In some embodiments, R⁶ is fluoro.

In some embodiments, R⁶ is H.

In some embodiments, R⁶ is chloro.

### the Group R⁷

In some embodiments, R⁷ is halogen.

In some embodiments, R⁷ is selected from: chloro and bromo.

In some embodiments, R⁷ is chloro.

In some embodiments, R⁷ is bromo.

### Certain Combinations

One aspect of the present invention is directed to compounds selected from compounds of Formula (Ic) and pharmaceutically acceptable salts, solvates, and hydrates thereof:

wherein:

R¹ is selected from: H, C₁-C₆ alkyl, C₁-C₆-alkyl-O—C₁-C₆-alkyl, C₃-C₇ cycloalkyl, C₄-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy;

R² is selected from: H and C₁-C₆ alkyl, wherein said C₁-C₆ alkyl is optionally substituted with one or more substituents selected from: hydroxyl and cyano; and

R⁴, R⁵, R⁶, and R⁷ are each selected independently from: H and halogen.

One aspect of the present invention is directed to compounds selected from compounds of Formula (Ic) and pharmaceutically acceptable salts, solvates, and hydrates thereof:

wherein:

R¹ is selected from: H, ethyl, 2-hydroxyethyl, 3-(1H-imidazol-1-yl)propyl, 4-methylpyridin-3-yl, methyl, 2-cyanoethyl, 2-amino-2-oxoethylamino, (1-methylpiperidin-4-yl)methyl, cyanomethyl, 1-amino-1-oxopropan-2-yl, 1,1-dioxo-tetrahydrothiophen-3-yl, 1-hydroxy-4-methylpentan-2-yl, 2-(1H-imidazol-5-yl)ethyl, (1-methyl-1H-imidazol-5-yl)methyl, 2-carbamoylcyclohexyl, 3-hydroxy-1-methoxy-1-oxopropan-2-yl, 1,3-dihydroxypropan-2-yl, 1-amino-3-hydroxy-1-oxopropan-2-yl, 2-hydroxycyclohexyl, 2-oxoazepan-3-yl, 2-(2-oxoimidazolidin-1-yl)ethyl, pyrrolidin-2-ylmethyl, pyrrolidin-3-yl, piperidin-3-yl, piperidin-4-yl, 2-hydroxypropyl, 2-hydroxypyridin-3-yl, 2-(4-methylpiperazin-1-yl)ethyl, 1-hydroxypropan-2-yl, 1,3-dihydroxy-2-(hydroxymethyl)propan-2-yl, 2-acetamidoethyl, 1-hydroxybutan-2-yl, 2-(1-methylpyrrolidin-2-yl)ethyl, 2-(dimethylamino)ethyl, 2-morpholinoethyl, 1-ethyl-2-oxoazepan-3-yl, 3-(dimethylamino)tetrahydrothiophen-3-yl)methyl, 2-(diethylamino)ethyl, 1-hydroxy-3-methylpentan-2-yl, 5-aminopentyl, 3-amino-1-imino-3-oxopropyl, (1-hydroxycyclohexyl)methyl, 2-(hydroxymethyl)pyrrolidin-1-yl)ethyl, 2-methyl-2-(piperidin-1-yl)propyl, benzyl, 2-(methylsulfinyl)ethyl, 2-(azepan-1-yl)ethyl, 3-hydroxybutyl, 1-amino-3-methyl-1-oxobutan-2-yl, 2-(2-(2-aminoethoxy)ethoxy)ethyl, 2-(hydroxymethyl)pyrrolidin-1-yl, 1,3-dihydroxybutan-2-yl, 2-morpholino-2-oxoethyl, 2-(dimethylamino)-2-(pyridin-3-yl)ethyl, 2-(pyrrolidin-1-yl)ethyl, 3-amino-1-methoxy-1-oxopropan-2-yl, 4-amino-1-methoxy-1-oxobutan-2-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, 2-hydroxycyclopentyl, piperidine-4-carbonyl, 2-aminocyclohexanecarbonyl, morpholine-2-carbonyl, 3-aminopropanoyl, 2-aminoacetyl, 4-hydroxypyrrolidine-2-carbonyl, 2-aminopropanoyl, 2-amino-3-hydroxypropanoyl, 2-hydroxyacetyl, thiomorpholine-3-carbonyl, pyrrolidine-2-carbonyl, 2-(morpholin-4-yl)acetyl, 2-(1H-tetrazol-5-yl)acetyl, 2-(dimethylamino)acetyl, 3-oxo-2,3-dihydroisoxazole-5-carbonyl, 6-oxo-1,6-dihydropyridazine-3-carbonyl, 2,4-dihydroxypyrimidine-5-carbonyl, 5-oxo-4,5-dihydro-1H-1,2,4-triazole-3-carbonyl, 4-aminotetrahydro-2H-thiopyran-4-carbonyl, 2-(3-amino-2-oxopyrrolidin-1-yl)acetyl, 6-hydroxynicotinoyl, 2-hydroxynicotinoyl, 2,6-dihydroxyisonicotinoyl, 2,6-dioxo-1,2,3,6-tetrahydropyrimidine-4-carbonyl, 5-hydroxy-1-methyl-1H-pyrazole-3-carbonyl, 3-(3-hydroxyisoxazol-4-yl)propanoyl, 3-carboxypropanoyl, 5-hydroxypyrazine-2-carbonyl, 6-hydroxypicolinoyl, 4-methylmorpholine-2-carbonyl, 4-ethylmorpholine-2-carbonyl, 4-(2-hydroxyethyl)morpholine-2-carbonyl, 4-(3,3-dimethylbutyl)morpholine-2-carbonyl, 4-(2-hydroxyethyl)morpholine-3-carbonyl, 4-ethylmorpholine-3-carbonyl, 4-(2-hydroxyethyl)thiomorpholine-3-carbonyl, 4-ethylthiomorpholine-3-carbonyl, 3-hydroxypropanoyl, 4-hydroxycyclohexanecarbonyl, 3-hydroxypentanoyl, 2-hydroxy-2-methylpropanoyl, 1-hydroxycyclopropanecarbonyl, 3-hydroxybutanoyl, 3-hydroxy-2,2-dimethylpropanoyl, 4-hydroxybutanoyl, 2-ethyl-2-hydroxybutanoyl, 2-hydroxycyclohexanecarbonyl, 2-cyclohexyl-2-hydroxyacetyl, 3-hydroxy-3-methylbutanoyl, 2-hydroxy-4-methylpentanoyl, 1-(tert-butoxycarbonyl)-4-hydroxypyrrolidine-2-carbonyl, 4-(tert-butoxycarbonyl)thiomorpholine-3-carbonyl, 2-(1-(tert-butoxycarbonyl)piperidin-2-yl)acetyl, 3-hydroxy-2-(hydroxymethyl)-2-methylpropanoyl, 2-(piperidin-2-yl)acetyl, 4-(hydroxymethyl)cyclohexanecarbonyl, 3-(dimethylamino)propanoyl, 2-(pyrrolidin-3-yl)acetyl, 3-(piperidin-1-yl)propanoyl, 4-aminocyclohexanecarbonyl, pyrrolidine-3-carbonyl, 3-(diethylamino)propanoyl, 2-(4-aminocyclohexyl)acetyl, 3-morpholinopropanoyl, 1-methylpiperidine-4-carbonyl, 3-aminocyclohexanecarbonyl, 2-amino-4-carboxybutanoyl, 4-amino-4-carboxybutanoyl, 3-aminocyclopentanecarbonyl, 1-methylpiperidine-3-carbonyl, 2-(piperidin-3-yl)acetyl, azetidine-3-carbonyl, 2-(4-(hydroxymethyl)piperidin-1-yl)acetyl, 2-(3-hydroxypiperidin-1-yl)acetyl, 2-(piperazin-1-yl)acetyl, 2-(3-aminopyrrolidin-1-yl)acetyl, 2-(2-(hydroxymethyl)morpholino)acetyl, 2-(4-propylpiperazin-1-yl)acetyl, 2-(5-oxo-1,4-diazepan-1-yl)acetyl, 2-(4-carbamoylpiperidin-1-yl)acetyl, 2-(2-carbamoylpyrrolidin-1-yl)acetyl, 2-(4-(dimethylamino)piperidin-1-yl)acetyl, 2-(3-(dimethylamino)pyrrolidin-1-yl)acetyl, 2-(4-hydroxypiperidin-1-yl)acetyl, 2-(2,5-diazabicyclo[2.2.1]heptan-2-yl)acetyl, 2-(hexahydropyrrolo[1,2-a]pyrazin-2(1H)-yl)acetyl, 2-(3-(hydroxymethyl)piperidin-1-yl)acetyl, 2-(3-methylpiperazin-1-yl)acetyl, 2-(4-methylpiperidin-1-yl)acetyl, 2-(3-oxopiperazin-1-yl)acetyl, 2-(4-carbamoylpiperazin-1-yl)acetyl, 2-(3-methylpiperidin-1-yl)acetyl, 2-(4-methylpiperazin-1-yl)acetyl, 2-(4-ethylpiperazin-1-yl)acetyl, 2-(2-(2-hydroxyethyl)piperidin-1-yl)acetyl, 2-(3-hydroxypyrrolidin-1-yl)acetyl, 2-(2-(hydroxymethyl)pyrrolidin-1-yl)acetyl, 2-(3-carbamoylpiperidin-1-yl)acetyl, 4-(phosphonooxy)cyclohexanecarbonyl, 2-(phosphonooxy)acetyl, 3-(tert-butoxycarbonylamino)pyrrolidine-1-carbonyl, 2-amino-4-methylpentanoyl, 2-amino-3-cyanopropanoyl, 4-amino-1,1-dioxotetrahydro-2H-thiopyran-4-carbonyl, 2,4-diamino-4-oxobutanoyl, 3-amino-2-hydroxypropanoyl, 2-hydroxypropanoyl, 5-(hydroxymethyl)-1H-1,2,3-triazole-4-carbonyl, piperazine-1-carbonyl, 4-ethylpiperazine-1-carbonyl, 1,1-dioxotetrahydro-2H-thiopyran-4-carbonyl, 3-hydroxypyrrolidine-1-carbonyl, 4-(2-hydroxyethyl)piperazine-1-carbonyl, 4-(hydroxymethyl)piperidine-1-carbonyl, 3-aminopiperidine-1-carbonyl, 3-hydroxyazetidine-1-carbonyl, 3-aminopyrrolidine-1-carbonyl, 2-carbamoylpyrrolidine-1-carbonyl, 4-(dimethylamino)piperidine-1-carbonyl, 4-carbamoylpiperazine-1-carbonyl, 3-oxopiperazine-1-carbonyl, 2-(hydroxymethyl)pyrrolidine-1-carbonyl, 2-(2-hydroxyethyl)piperidine-1-carbonyl, 2-(hydroxymethyl)morpholine-4-carbonyl, 3-carboxyazetidine-1-carbonyl, 4-(3-hydroxypropyl)piperidine-1-carbonyl, 3-hydroxypiperidine-1-carbonyl, 4-cyanopiperidine-1-carbonyl, 2-(hydroxymethyl)piperidine-1-carbonyl, 4-hydroxypiperidine-1-carbonyl, 2-oxopyrrolidine-1-carbonyl, 3-(hydroxymethyl)piperidine-1-carbonyl, 3-(hydroxymethyl)pyrrolidine-1-carbonyl, 3-(phosphonooxy)pyrrolidine-1-carbonyl, 1-(tert-butoxycarbonyl)piperidine-4-carbonyl, 2-(tert-butoxycarbonylamino)-cyclohexanecarbonyl, 1-(tert-butoxycarbonyl)piperidine-3-carbonyl, 3-(tert-butoxycarbonylamino)piperidine-1-carbonyl, 4-(tert-butoxycarbonyl)morpholine-2-carbonyl, 3-(tert-butoxycarbonylamino)propanoyl, 2-(tert-butoxycarbonylamino)acetyl, 3-(tert-butoxycarbonylamino)-2-hydroxypropanoyl, 2-(tert-butoxycarbonylamino)propanoyl, 2-(tert-butoxycarbonylamino)-3-hydroxypropanoyl, 1-(tert-butoxycarbonyl)pyrrolidine-2-carbonyl, 4-(tert-butoxycarbonylamino)tetrahydro-2H-thiopyran-4-carbonyl, 4-tert-butoxy-4-oxobutanoyl, 2-(3-(tert-butoxycarbonylamino)-2-oxopyrrolidin-1-yl)acetyl, 4-amino-2-(tert-butoxycarbonylamino)-4-oxobutanoyl, 2-(1-(tert-butoxycarbonyl)pyrrolidin-3-yl)acetyl, 4-(tert-butoxycarbonylamino)cyclohexanecarbonyl, 1-(tert-butoxycarbonyl)pyrrolidine-3-carbonyl, 2-(4-(tert-butoxycarbonylamino)cyclohexyl)acetyl, 3-(tert-butoxycarbonylamino)cyclohexanecarbonyl, 2-(tert-butoxycarbonylamino)-4-carboxybutanoyl, 4-(tert-butoxycarbonylamino)-4-carboxybutanoyl, 3-(tert-butoxycarbonylamino)cyclopentanecarbonyl, 2-(1-(tert-butoxycarbonyl)piperidin-3-yl)acetyl, 1-(tert-butoxycarbonyl)azetidine-3-carbonyl, 2-(3-(tert-butoxycarbonylamino)pyrrolidin-1-yl, 2-(4-(tert-butoxycarbonyl)-3-methylpiperazin-1-yl)acetyl, 2-(tert-butoxycarbonylamino)-4-methylpentanoyl, 2-(tert-butoxycarbonylamino)-3-cyanopropanoyl, and 4-(tert-butoxycarbonylamino)-1,1-dioxotetrahydro-2H-thiopyran-4-carbonyl;

R² is selected from: H, ethyl, methyl, isopropyl, 2-hydroxyethyl, 2-cyanoethyl, and tert-butyl;

R⁴ is selected from: H, fluoro, and chloro;

R⁵ is selected from: H and fluoro;

R⁶ is selected from: H, fluoro, and chloro; and

R⁷ is selected from: bromo and chloro.

One aspect of the present invention is directed to compounds selected from compounds of Formula (Ic) and pharmaceutically acceptable salts, solvates, and hydrates thereof:

wherein:

R¹ is selected from: H, ethyl, 2-hydroxyethyl, 3-(1H-imidazol-1-yl)propyl, 4-methylpyridin-3-yl, methyl, 2-cyanoethyl, 2-amino-2-oxoethylamino, (1-methylpiperidin-4-yl)methyl, cyanomethyl, 1-amino-1-oxopropan-2-yl, 1,1-dioxo-tetrahydrothiophen-3-yl, 1-hydroxy-4-methylpentan-2-yl, 2-(1H-imidazol-5-yl)ethyl, (1-methyl-1H-imidazol-5-yl)methyl, 2-carbamoylcyclohexyl, 3-hydroxy-1-methoxy-1-oxopropan-2-yl, 1,3-dihydroxypropan-2-yl, 1-amino-3-hydroxy-1-oxopropan-2-yl, 2-hydroxycyclohexyl, 2-oxoazepan-3-yl, 2-(2-oxoimidazolidin-1-yl)ethyl, pyrrolidin-2-ylmethyl, pyrrolidin-3-yl, piperidin-3-yl, piperidin-4-yl, 2-hydroxypropyl, 2-hydroxypyridin-3-yl, 2-(4-methylpiperazin-1-yl)ethyl, 1-hydroxypropan-2-yl, 1,3-dihydroxy-2-(hydroxymethyl)propan-2-yl, 2-acetamidoethyl, 1-hydroxybutan-2-yl, 2-(1-methylpyrrolidin-2-yl)ethyl, 2-(dimethylamino)ethyl, 2-morpholinoethyl, 1-ethyl-2-oxoazepan-3-yl, 3-(dimethylamino)tetrahydrothiophen-3-yl)methyl, 2-(diethylamino)ethyl, 1-hydroxy-3-methylpentan-2-yl, 5-aminopentyl, 3-amino-1-imino-3-oxopropyl, (1-hydroxycyclohexyl)methyl, 2-(hydroxymethyl)pyrrolidin-1-yl)ethyl, 2-methyl-2-(piperidin-1-yl)propyl, benzyl, 2-(methylsulfinyl)ethyl, 2-(azepan-1-yl)ethyl, 3-hydroxybutyl, 1-amino-3-methyl-1-oxobutan-2-yl, 2-(2-(2-aminoethoxy)ethoxy)ethyl, 2-(hydroxymethyl)pyrrolidin-1-yl, 1,3-dihydroxybutan-2-yl, 2-morpholino-2-oxoethyl, 2-(dimethylamino)-2-(pyridin-3-yl)ethyl, 2-(pyrrolidin-1-yl)ethyl, 3-amino-1-methoxy-1-oxopropan-2-yl, 4-amino-1-methoxy-1-oxobutan-2-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, and 2-hydroxycyclopentyl;

R² is selected from: H, ethyl, methyl, isopropyl, 2-hydroxyethyl, 2-cyanoethyl, and tert-butyl;

R⁴ is selected from: H, fluoro, and chloro;

R⁵ is selected from: H and fluoro;

R⁶ is selected from: H and chloro; and

R⁷ is selected from: bromo and chloro.

One aspect of the present invention is directed to compounds selected from compounds of Formula (Ie) and pharmaceutically acceptable salts, solvates, and hydrates thereof:

wherein:

R¹ and R² together with the nitrogen atom to which they are both bonded form a group selected from: heteroaryl and heterocyclyl, each optionally substituted with one or more substituents selected from: C₁-C₆ alkoxycarbonyl, C₁-C₆ alkoxycarbonylamino, C₁-C₆ alkyl, C₁-C₆ alkylcarboxamide, C₁-C₆ alkylsulfonyl, amino, C₃-C₇ cycloalkyl, C₄-C₁₃ cycloalkylalkyl, carboxamide, carboxyl, C₂-C₆ dialkylamino, C₂-C₆ dialkylcarboxamide, heteroaryl-C₁-C₆-alkyl, heterocyclyl, heterocyclyl-C₁-C₆-alkyl, hydroxyl, hydroxyheterocyclyl, and oxo, wherein said C₁-C₆ alkyl and C₁-C₆ alkylcarboxamide are each optionally substituted with one or more substituents selected from: carboxyl, hydroxyl, and oxo; and

R⁴, R⁵, and R⁶ are each selected independently from: H and halogen.

One aspect of the present invention is directed to compounds selected from compounds of Formula (Ie) and pharmaceutically acceptable salts, solvates, and hydrates thereof:

wherein:

R¹ and R² together with the nitrogen atom to which they are both bonded form a group selected from: 1,1-dioxo-thiomorpholin-4-yl, 3-hydroxypyrrolidin-1-yl, 4-(2-hydroxyethyl)piperazin-1-yl, hexahydropyrrolo[1,2-a]pyrazin-2(1H)-yl, 4-ethylpiperazin-1-yl, piperidin-1-yl, 1H-imidazol-1-yl, morpholino, 4-methylpiperazin-1-yl, pyrrolidin-1-yl, 1H-1,2,4-triazol-1-yl, 1H-pyrazol-1-yl, 1H-pyrrol-1-yl, 2H-tetrazol-5-yl, piperazin-1-yl, 4-(dimethylamino)piperidin-1-yl, 4-(hydroxymethyl)piperidin-1-yl, 2-carbamoylpyrrolidin-1-yl, 2-(2-hydroxyethyl)piperidin-1-yl, 4-carbamoylpiperazin-1-yl, 3-oxopiperazin-1-yl, 4-(2-cyclohexylethyl)piperazin-1-yl, 2,7-diazaspiro[4.4]nonan-2-yl, 3-(methylsulfonyl)pyrrolidin-1-yl, 6,7-dihydro-1H-imidazo[4,5-c]pyridin-5(4H)-yl, 2-(hydroxymethyl)piperidin-1-yl, 3-aminopyrrolidin-1-yl, 2-methylpiperazin-1-yl, 3-aminopiperidin-1-yl, 4-aminopiperidin-1-yl, 2-carbamoylpiperidin-1-yl, 5,6-dihydropyrimidin-1(4H)-yl, 4-hydroxy-2-(methoxycarbonyl)pyrrolidin-1-yl, 4-hydroxypiperidin-1-yl, 4-(2-(pyridin-2-yl)ethyl)piperazin-1-yl, 3-hydroxypiperidin-1-yl, 3-(diethylcarbamoyl)piperidin-1-yl, 2,3,4,6,7,8-hexahydro-1H-pyrimido[1,2-a]pyrimidin-1-yl, 4-cyclopentylpiperazin-1-yl, 1,4-oxazepan-4-yl, 2-(pyrrolidin-1-ylmethyl)pyrrolidin-1-yl, 4-morpholinopiperidin-1-yl, 4-(cyclohexylmethyl)piperazin-1-yl, 4-oxopiperidin-1-yl, 4-acetylpiperazin-1-yl, 1,4′-bipiperidin-1′-yl, 4-(ethoxycarbonyl)piperidin-1-yl, 2-(hydroxymethyl)morpholino, 2-(hydroxymethyl)pyrrolidin-1-yl, 3-hydroxyazetidin-1-yl, 4-hydroxy-1,4′-bipiperidin-1′-yl, 3-(hydroxymethyl)piperidin-1-yl, 2,5-diazabicyclo[2.2.1]heptan-2-yl, 5-oxo-1,4-diazepan-1-yl, 4-(2-hydroxyethyl)piperidin-1-yl, 3-(carboxymethyl)pyrrolidin-1-yl, 2,7-diazaspiro[3.5]nonan-2-yl, 4-(tert-butoxycarbonyl)-2-(carboxymethyl)piperazin-1-yl, 4-(tert-butoxycarbonyl)-2-carboxypiperazin-1-yl, 4-carboxypiperidin-1-yl, 2-(carboxymethyl)morpholino, 2-(carboxymethyl)piperazin-1-yl, 2-carboxypiperazin-1-yl, 4-(carboxymethyl)piperazin-1-yl, 2-carboxy-5,6-dihydroimidazo[1,2-a]pyrazin-7(8H)-yl, 2-carbamoylpiperazin-1-yl, 2-(methylcarbamoyl)piperazin-1-yl, 2-(2-hydroxyethylcarbamoyl)piperazin-1-yl, 2-(1-hydroxypropan-2-ylcarbamoyl)piperazin-1-yl, 3-carbamoylpiperidin-1-yl, 4-carbamoylpiperidin-1-yl, 3-(hydroxymethyl)pyrrolidin-1-yl, 2-oxopyrrolidin-1-yl, 2,5-dioxoimidazolidin-1-yl, 2,6-dioxotetrahydropyrimidin-1(2H)-yl, 3-methyl-2,5-dioxoimidazolidin-1-yl, and 4-isobutyl-2,5-dioxoimidazolidin-1-yl;

R⁴ is selected from: H and fluoro;

R⁵ is selected from: H and fluoro; and

R⁶ is selected from: H and chloro.

One aspect of the present invention is directed to compounds selected from compounds of Formula (Ie) and pharmaceutically acceptable salts, solvates, and hydrates thereof:

wherein:

R¹ and R² together with the nitrogen atom to which they are both bonded form a group selected from: 1,1-dioxo-thiomorpholin-4-yl, 3-hydroxypyrrolidin-1-yl, 4-(2-hydroxyethyl)piperazin-1-yl, hexahydropyrrolo[1,2-a]pyrazin-2(1H)-yl, 4-ethylpiperazin-1-yl, piperidin-1-yl, 1H-imidazol-1-yl, morpholino, 4-methylpiperazin-1-yl, pyrrolidin-1-yl, 1H-1,2,4-triazol-1-yl, 1H-pyrazol-1-yl, 1H-pyrrol-1-yl, 2H-tetrazol-5-yl, piperazin-1-yl, 4-(dimethylamino)piperidin-1-yl, 4-(hydroxymethyl)piperidin-1-yl, 2-carbamoylpyrrolidin-1-yl, 2-(2-hydroxyethyl)piperidin-1-yl, 4-carbamoylpiperazin-1-yl, 3-oxopiperazin-1-yl, 4-(2-cyclohexylethyl)piperazin-1-yl, 2,7-diazaspiro[4.4]nonan-2-yl, 3-(methylsulfonyl)pyrrolidin-1-yl, 6,7-dihydro-1H-imidazo[4,5-c]pyridin-5(4H)-yl, 2-(hydroxymethyl)piperidin-1-yl, 3-aminopyrrolidin-1-yl, 2-methylpiperazin-1-yl, 3-aminopiperidin-1-yl, 4-aminopiperidin-1-yl, 2-carbamoylpiperidin-1-yl, 5,6-dihydropyrimidin-1(4H)-yl, 4-hydroxy-2-(methoxycarbonyl)pyrrolidin-1-yl, 4-hydroxypiperidin-1-yl, 4-(2-(pyridin-2-yl)ethyl)piperazin-1-yl, 3-hydroxypiperidin-1-yl, 3-(diethylcarbamoyl)piperidin-1-yl, 2,3,4,6,7,8-hexahydro-1H-pyrimido[1,2-a]pyrimidin-1-yl, 4-cyclopentylpiperazin-1-yl, 1,4-oxazepan-4-yl, 2-(pyrrolidin-1-ylmethyl)pyrrolidin-1-yl, 4-morpholinopiperidin-1-yl, 4-(cyclohexylmethyl)piperazin-1-yl, 4-oxopiperidin-1-yl, 4-acetylpiperazin-1-yl, 1,4′-bipiperidin-1′-yl, 4-(ethoxycarbonyl)piperidin-1-yl, 2-(hydroxymethyl)morpholino, 2-(hydroxymethyl)pyrrolidin-1-yl, 3-hydroxyazetidin-1-yl, 4-hydroxy-1,4′-bipiperidin-1′-yl, 3-(hydroxymethyl)piperidin-1-yl, 2,5-diazabicyclo[2.2.1]heptan-2-yl, 5-oxo-1,4-diazepan-1-yl, 4-(2-hydroxyethyl)piperidin-1-yl, 3-(carboxymethyl)pyrrolidin-1-yl, 2,7-diazaspiro[3.5]nonan-2-yl, 4-(tert-butoxycarbonyl)-2-(carboxymethyl)piperazin-1-yl, 4-(tert-butoxycarbonyl)-2-carboxypiperazin-1-yl, 4-carboxypiperidin-1-yl, 2-(carboxymethyl)morpholino, 2-(carboxymethyl)piperazin-1-yl, 2-carboxypiperazin-1-yl, 4-(carboxymethyl)piperazin-1-yl, 2-carboxy-5,6-dihydroimidazo[1,2-a]pyrazin-7(8H)-yl, 2-carbamoylpiperazin-1-yl, 2-(methylcarbamoyl)piperazin-1-yl, 2-(2-hydroxyethylcarbamoyl)piperazin-1-yl, 2-(1-hydroxypropan-2-ylcarbamoyl)piperazin-1-yl, 3-carbamoylpiperidin-1-yl, 4-carbamoylpiperidin-1-yl, and 3-(hydroxymethyl)pyrrolidin-1-yl;

R⁴ is selected from: H and fluoro;

R⁵ is selected from: H and fluoro; and

R⁶ is selected from: H and chloro.

One aspect of the present invention is directed to compounds selected from compounds of Formula (Ig) and pharmaceutically acceptable salts, solvates, and hydrates thereof:

wherein:

X is CH₂ or CH₂CH₂;

R¹ is selected from: H, C₁-C₆ alkyl, C₁-C₆-alkyl-O—C₁-C₆-alkyl, and heterocyclyl-C₁-C₆-alkyl, each optionally substituted with one or more substituents selected from: C₁-C₆ alkoxycarbonyl, C₁-C₆ alkylsulfonyl, carboxamide, cyano, C₂-C₆ dialkylamino, and hydroxyl; and

R² and R³ together form CH₂.

One aspect of the present invention is directed to compounds selected from compounds of Formula (Ig) and pharmaceutically acceptable salts, solvates, and hydrates thereof:

wherein:

X is CH₂ or CH₂CH₂;

R¹ is selected from: H, methyl, butyl, 3-hydroxypropyl, 3,3-dimethylbutyl, (tetrahydro-2H-pyran-4-yl)methyl, 2-methoxyethyl, 3-amino-3-oxopropyl, 2-hydroxyethyl, 2-ethoxy-2-oxoethyl, 2-amino-2-oxoethyl, cyanomethyl, 2-ethoxyethyl, 2-(diethylamino)ethyl, 2-(methylsulfonyl)ethyl, butyr-1-yl, 2-ethylbutanoyl, thiophene-2-carbonyl, nicotinoyl, and 2-cyclopentylacetyl;

R² and R³ together form CH₂.

One aspect of the present invention is directed to compounds selected from compounds of Formula (Ig) and pharmaceutically acceptable salts, solvates, and hydrates thereof:

wherein:

X is CH₂ or CH₂CH₂;

R¹ is selected from: H, methyl, butyl, 3-hydroxypropyl, 3,3-dimethylbutyl, (tetrahydro-2H-pyran-4-yl)methyl, 2-methoxyethyl, 3-amino-3-oxopropyl, 2-hydroxyethyl, 2-ethoxy-2-oxoethyl, 2-amino-2-oxoethyl, cyanomethyl, 2-ethoxyethyl, 2-(diethylamino)ethyl, and 2-(methylsulfonyl)ethyl; and

R² and R³ together form CH₂.

One aspect of the present invention is directed to compounds selected from compounds of Formula (Ii) and pharmaceutically acceptable salts, solvates, and hydrates thereof:

wherein:

R¹ is selected from: H, C₁-C₆ alkyl, C₃-C₇ cycloalkyl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, C₁-C₆ alkoxycarbonyl, amino, carboxamide, carboxyl, cyano, hydroxyl, hydroxy-C₁-C₆-alkyl, oxo, and phosphonooxy; and

R² is selected from: H and C₁-C₆ alkyl, wherein said C₁-C₆ alkyl is optionally substituted with one or more hydroxyl substituents.

One aspect of the present invention is directed to compounds selected from compounds of Formula (Ii) and pharmaceutically acceptable salts, solvates, and hydrates thereof:

wherein:

R¹ is selected from: H, 2-hydroxyethyl, 2-cyanoethyl, 1,1-dioxo-tetrahydrothiophen-3-yl, 2-carbamoylcyclohexyl, 1-amino-3-hydroxy-1-oxopropan-2-yl, 2-hydroxycyclohexyl, piperidin-3-yl, piperidin-4-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, and 2-hydroxycyclopentyl; and

R² is selected from: H, ethyl, methyl, and 2-hydroxyethyl.

One aspect of the present invention is directed to compounds selected from compounds of Formula (Ii) and pharmaceutically acceptable salts, solvates, and hydrates thereof:

wherein:

R¹ and R² together with the nitrogen atom to which they are both bonded form a group selected from: heteroaryl and heterocyclyl, each optionally substituted with one or more substituents selected from: C₁-C₆ alkoxycarbonyl, C₁-C₆ alkyl, C₁-C₆ alkylcarboxamide, carboxamide, carboxyl, hydroxyl, and oxo, wherein said C₁-C₆ alkyl and C₁-C₆ alkylcarboxamide are each optionally substituted with one or more substituents selected from: carboxyl and hydroxyl.

One aspect of the present invention is directed to compounds selected from compounds of Formula (Ii) and pharmaceutically acceptable salts, solvates, and hydrates thereof:

wherein:

R¹ and R² together with the nitrogen atom to which they are both bonded form a group selected from: 1,1-dioxo-thiomorpholin-4-yl, 3-hydroxypyrrolidin-1-yl, 4-(2-hydroxyethyl)piperazin-1-yl, piperazin-1-yl, 4-(hydroxymethyl)piperidin-1-yl, 2-carbamoylpyrrolidin-1-yl, 2-(2-hydroxyethyl)piperidin-1-yl, 4-carbamoylpiperazin-1-yl, 3-oxopiperazin-1-yl, 4-hydroxypiperidin-1-yl, 3-hydroxypiperidin-1-yl, 2-(hydroxymethyl)morpholino, 2-(hydroxymethyl)pyrrolidin-1-yl, 3-hydroxyazetidin-1-yl, 4-(2-hydroxyethyl)piperidin-1-yl, 3-(carboxymethyl)pyrrolidin-1-yl, 4-(tert-butoxycarbonyl)-2-(carboxymethyl)piperazin-1-yl, 4-(tert-butoxycarbonyl)-2-carboxypiperazin-1-yl, 4-carboxypiperidin-1-yl, 2-(carboxymethyl)morpholino, 2-(carboxymethyl)piperazin-1-yl, 2-carboxypiperazin-1-yl, 4-(carboxymethyl)piperazin-1-yl, 2-carboxy-5,6-dihydroimidazo[1,2-a]pyrazin-7(8H)-yl, 2-carbamoylpiperazin-1-yl, 2-(methylcarbamoyl)piperazin-1-yl, 2-(2-hydroxyethylcarbamoyl)piperazin-1-yl, 2-(1-hydroxypropan-2-ylcarbamoyl)piperazin-1-yl, 3-carbamoylpiperidin-1-yl, 4-carbamoylpiperidin-1-yl, and 3-(hydroxymethyl)pyrrolidin-1-yl.

One aspect of the present invention is directed to compounds selected from compounds of Formula (Ik) and pharmaceutically acceptable salts, solvates, and hydrates thereof:

wherein:

R¹ is selected from: C₁-C₆ alkyl, C₄-C₁₃ cycloalkylalkyl, heteroaryl-C₁-C₆-alkyl, and heterocyclyl-C₁-C₆-alkyl, each optionally substituted with one or more substituents selected from: amino, carboxamide, hydroxyl, hydroxy-C₁-C₆-alkyl, oxo, and phosphonooxy; and

R² is H; or

R¹ and R² together with the nitrogen atom to which they are both bonded form a heterocyclyl group optionally substituted with one or more oxo substituents; and

R⁴ and R⁶ are each selected independently from: H and halogen.

One aspect of the present invention is directed to compounds selected from compounds of Formula (Ik) and pharmaceutically acceptable salts, solvates, and hydrates thereof:

wherein:

R¹ is selected from: ethyl, propan-1-yl, propan-2-yl, butan-1-yl, isobutyl, morpholin-2-ylmethyl, 2-(morpholin-4-yl)ethyl, (4,5-dihydro-1H-1,2,4-triazol-3-yl)methyl, pyridin-3-ylmethyl, pyrazin-2-ylmethyl, cyclohexylmethyl, 4-methylpentyl, pyrrolidin-1-ylmethyl, (1,1-dioxotetrahydro-2H-thiopyran-4-yl)methyl, piperidin-1-ylmethyl, piperazin-1-ylmethyl, azetidin-1-ylmethyl, and (morpholin-4-yl)methyl; each optionally substituted with one or more substituents selected from: amino, carboxamide, hydroxyl, hydroxymethyl, oxo, and phosphonooxy; and

R² is H; or

R¹ and R² together with the nitrogen atom to which they are both bonded form a piperazinyl group optionally substituted with one or more oxo substituents; and

R⁴ and R⁶ are each selected independently from: H, fluoro, and chloro.

One aspect of the present invention is directed to compounds selected from compounds of Formula (Ik) and pharmaceutically acceptable salts, solvates, and hydrates thereof:

wherein:

R¹ is selected from: 1-amino-3-hydroxy-1-oxopropan-2-yl, 1-amino-1-oxo-3-(phosphonooxy)propan-2-yl, 2-hydroxyacetyl, morpholine-2-carbonyl, 2-(morpholin-4-yl)acetyl, 5-oxo-4,5-dihydro-1H-1,2,4-triazole-3-carbonyl, 2-hydroxynicotinoyl, 5-hydroxypyrazine-2-carbonyl, 4-hydroxycyclohexanecarbonyl, 2-hydroxy-2-methylpropanoyl, 1-hydroxycyclopropanecarbonyl, 3-hydroxybutanoyl, 2-hydroxy-4-methylpentanoyl, 3-hydroxy-2-(hydroxymethyl)-2-methylpropanoyl, 4-(hydroxymethyl)cyclohexanecarbonyl, 4-(phosphonooxy)cyclohexanecarbonyl, 2-(phosphonooxy)acetyl, 4-amino-1,1-dioxotetrahydro-2H-thiopyran-4-carbonyl, 2-hydroxypropanoyl, 3-hydroxypyrrolidine-1-carbonyl, 4-(hydroxymethyl)piperidine-1-carbonyl, 3-hydroxyazetidine-1-carbonyl, 2-carbamoylpyrrolidine-1-carbonyl, 3-oxopiperazine-1-carbonyl, 2-(hydroxymethyl)pyrrolidine-1-carbonyl, 2-(hydroxymethyl)morpholine-4-carbonyl, 3-hydroxypiperidine-1-carbonyl, 4-hydroxypiperidine-1-carbonyl, 3-(phosphonooxy)pyrrolidine-1-carbonyl, and 3-(hydroxymethyl)pyrrolidine-1-carbonyl; and

R² is H; or

R¹ and R² together with the nitrogen atom to which they are both bonded form a 3-oxopiperazin-1-yl group;

R⁴ is selected from: H and fluoro; and

R⁶ is selected from: H and chloro. Some embodiments of the present invention include every combination of one or more compounds selected from the following group, where the number in the parentheses directly preceding the chemical name refers to the Compound Number that is used throughout this disclosure: (#1), N-(4-Chloro-2-(4-(3,3,3-trifluoropropyl)piperazin-1-yl)phenyl)-4-((1,1-dioxo-thiomorpholin-4-yl)methyl)benzamide; (#2), N-(4-Chloro-2-(4-(3,3,3-trifluoropropyl)piperazin-1-yl)phenyl)-4-((diethylamino)methyl)benzamide; (#3), N-(4-Chloro-2-(4-(3,3,3-trifluoropropyl)piperazin-1-yl)phenyl)-4-((2-hydroxyethylamino)methyl)benzamide; (#4), N-(4-Chloro-2-(4-(3,3,3-trifluoropropyl)piperazin-1-yl)phenyl)-4-((ethylamino)methyl)benzamide; (#5), (S)—N-(4-Chloro-2-(4-(3,3,3-trifluoropropyl)piperazin-1-yl)phenyl)-4-((3-hydroxypyrrolidin-1-yl)methyl)benzamide; (#6), N-(4-Chloro-2-(4-(3,3,3-trifluoropropyl)piperazin-1-yl)phenyl)-4-((4-(2-hydroxyethyl)piperazin-1-yl)methyl)benzamide; (#7), (S)—N-(4-Chloro-2-(4-(3,3,3-trifluoropropyl)piperazin-1-yl)phenyl)-4-((hexahydropyrrolo[1,2-a]pyrazin-2(1H)-yl)methyl)benzamide; (#8), N-(4-Chloro-2-(4-(3,3,3-trifluoropropyl)piperazin-1-yl)phenyl)-4-(((2-hydroxyethyl)(methyl)amino)methyl)benzamide; (#9), N-(4-Chloro-2-(4-(3,3,3-trifluoropropyl)piperazin-1-yl)phenyl)-4-((4-ethylpiperazin-1-yl)methyl)benzamide; (#10), 4-((3-(1H-Imidazol-1-yl)propylamino)methyl)-N-(4-chloro-2-(4-(3,3,3-trifluoropropyl)piperazin-1-yl)phenyl)benzamide; (#11), N-(4-Chloro-2-(4-(3,3,3-trifluoropropyl)piperazin-1-yl)phenyl)-4-((4-methylpyridin-3-ylamino)methyl)benzamide; (#12), N-(4-Chloro-2-(4-(3,3,3-trifluoropropyl)piperazin-1-yl)phenyl)-4-(piperidin-1-ylmethyl)benzamide; (#13), N-(4-Chloro-2-(4-(3,3,3-trifluoropropyl)piperazin-1-yl)phenyl)-1,2,3,4-tetrahydroisoquinoline-7-carboxamide; (#14), N-(4-Chloro-2-(4-(3,3,3-trifluoropropyl)piperazin-1-yl)phenyl)-2,3-difluoro-4-(((1R,2R)-2-hydroxycyclopentylamino)methyl)benzamide; (#15), 2-Butyl-N-(4-chloro-2-(4-(3,3,3-trifluoropropyl)piperazin-1-yl)phenyl)-1,2,3,4-tetrahydroisoquinoline-7-carboxamide; (#16), N-(4-Chloro-2-(4-(3,3,3-trifluoropropyl)piperazin-1-yl)phenyl)-2-(3-hydroxypropyl)-1,2,3,4-tetrahydroisoquinoline-7-carboxamide; (#17), N-(4-Chloro-2-(4-(3,3,3-trifluoropropyl)piperazin-1-yl)phenyl)-2-(3,3-dimethylbutyl)-1,2,3,4-tetrahydroisoquinoline-7-carboxamide; (#18), N-(4-Chloro-2-(4-(3,3,3-trifluoropropyl)piperazin-1-yl)phenyl)-2-((tetrahydro-2H-pyran-4-yl)methyl)-1,2,3,4-tetrahydroisoquinoline-7-carboxamide; (#19), N-(4-Chloro-2-(4-(3,3,3-trifluoropropyl)piperazin-1-yl)phenyl)-2-(2-methoxyethyl)-1,2,3,4-tetrahydroisoquinoline-7-carboxamide; (#20), 2-(3-Amino-3-oxopropyl)-N-(4-chloro-2-(4-(3,3,3-trifluoropropyl)piperazin-1-yl)phenyl)-1,2,3,4-tetrahydroisoquinoline-7-carboxamide; (#21), N-(4-Chloro-2-(4-(3,3,3-trifluoropropyl)piperazin-1-yl)phenyl)-2-(2-hydroxyethyl)-1,2,3,4-tetrahydroisoquinoline-7-carboxamide; (#22), Ethyl 2-(7-(4-chloro-2-(4-(3,3,3-trifluoropropyl)piperazin-1-yl)phenylcarbamoyl)-3,4-dihydroisoquinolin-2(1H)-yl)acetate; (#23), 2-(2-Amino-2-oxoethyl)-N-(4-chloro-2-(4-(3,3,3-trifluoropropyl)piperazin-1-yl)phenyl)-1,2,3,4-tetrahydroisoquinoline-7-carboxamide; (#24), N-(4-Chloro-2-(4-(3,3,3-trifluoropropyl)piperazin-1-yl)phenyl)-2-(cyanomethyl)-1,2,3,4-tetrahydroisoquinoline-7-carboxamide; (#25), N-(4-Chloro-2-(4-(3,3,3-trifluoropropyl)piperazin-1-yl)phenyl)isoindoline-5-carboxamide; (#26), 4-(Aminomethyl)-N-(4-chloro-2-(4-(3,3,3-trifluoropropyl)piperazin-1-yl)phenyl)-2-fluorobenzamide; (#27), Ethyl 2-(5-(4-chloro-2-(4-(3,3,3-trifluoropropyl)piperazin-1-yl)phenylcarbamoyl)isoindolin-2-yl)acetate; (#28), N-(4-Chloro-2-(4-(3,3,3-trifluoropropyl)piperazin-1-yl)phenyl)-2-(2-hydroxyethyl)isoindoline-5-carboxamide; (#29), N-(4-Chloro-2-(4-(3,3,3-trifluoropropyl)piperazin-1-yl)phenyl)-2-(cyanomethyl)isoindoline-5-carboxamide; (#30), 2-(2-Amino-2-oxoethyl)-N-(4-chloro-2-(4-(3,3,3-trifluoropropyl)piperazin-1-yl)phenyl)isoindoline-5-carboxamide; (#31), N-(4-Chloro-2-(4-(3,3,3-trifluoropropyl)piperazin-1-yl)phenyl)-2-(2-ethoxyethyl)isoindoline-5-carboxamide; (#32), N-(4-Chloro-2-(4-(3,3,3-trifluoropropyl)piperazin-1-yl)phenyl)-2-(2-(diethylamino)ethyl)isoindoline-5-carboxamide; (#33), 2-(3-Amino-3-oxopropyl)-N-(4-chloro-2-(4-(3,3,3-trifluoropropyl)piperazin-1-yl)phenyl)isoindoline-5-carboxamide; (#34), N-(4-Chloro-2-(4-(3,3,3-trifluoropropyl)piperazin-1-yl)phenyl)-4-(diethylamino)benzamide; (#35), N-(4-Chloro-2-(4-(3,3,3-trifluoropropyl)piperazin-1-yl)phenyl)-4-(dimethylamino)benzamide; (#36), N-(4-Chloro-2-(4-(3,3,3-trifluoropropyl)piperazin-1-yl)phenyl)-2-(2-(methylsulfonyl)ethyl)-1,2,3,4-tetrahydroisoquinoline-7-carboxamide; (#37), N-(4-Chloro-2-(4-(3,3,3-trifluoropropyl)piperazin-1-yl)phenyl)-2-(2-(methylsulfonyl)ethyl)isoindoline-5-carboxamide; (#38), N-(4-Chloro-2-(4-(3,3,3-trifluoropropyl)piperazin-1-yl)phenyl)-4-(1H-imidazol-1-yl)benzamide; (#39), N-(4-Chloro-2-(4-(3,3,3-trifluoropropyl)piperazin-1-yl)phenyl)-4-(morpholinomethyl)benzamide; (#40), N-(4-Chloro-2-(4-(3,3,3-trifluoropropyl)piperazin-1-yl)phenyl)-4-(4-methylpiperazin-1-yl)benzamide; (#41), N-(4-Chloro-2-(4-(3,3,3-trifluoropropyl)piperazin-1-yl)phenyl)-4-(pyrrolidin-1-yl)benzamide; (#42), N-(4-Chloro-2-(4-(3,3,3-trifluoropropyl)piperazin-1-yl)phenyl)-4-morpholinobenzamide; (#43), N-(4-Chloro-2-(4-(3,3,3-trifluoropropyl)piperazin-1-yl)phenyl)-4-(1H-1,2,4-triazol-1-yl)benzamide; (#44), N-(4-Chloro-2-(4-(3,3,3-trifluoropropyl)piperazin-1-yl)phenyl)-4-(pyrrolidin-1-ylmethyl)benzamide; (#45), N-(4-Chloro-2-(4-(3,3,3-trifluoropropyl)piperazin-1-yl)phenyl)-4-(1H-pyrazol-1-yl)benzamide; (#46), N-(4-Chloro-2-(4-(3,3,3-trifluoropropyl)piperazin-1-yl)phenyl)-4-(1H-pyrrol-1-yl)benzamide; (#47), N-(4-Chloro-2-(4-(3,3,3-trifluoropropyl)piperazin-1-yl)phenyl)-2,3-difluoro-4-(((1R,2S)-2-hydroxycyclopentylamino)methyl)benzamide; (#48), 4-(Aminomethyl)-N-(4-chloro-2-(4-(3,3,3-trifluoropropyl)piperazin-1-yl)phenyl)-2,3-difluorobenzamide; (#49), N-(4-Chloro-2-(4-(3,3,3-trifluoropropyl)piperazin-1-yl)phenyl)-2-fluoro-4-(piperazin-1-ylmethyl)benzamide; (#50), N-(4-Chloro-2-(4-(3,3,3-trifluoropropyl)piperazin-1-yl)phenyl)-2-fluoro-4-((1,1-dioxo-thiomorpholin-4-yl)methyl)benzamide; (#51), (S)—N-(4-Chloro-2-(4-(3,3,3-trifluoropropyl)piperazin-1-yl)phenyl)-2-fluoro-4-((3-hydroxypyrrolidin-1-yl)methyl)benzamide; (#52), N-(4-Chloro-2-(4-(3,3,3-trifluoropropyl)piperazin-1-yl)phenyl)-4-(((2-cyanoethyl)(methyl)amino)methyl)-2-fluorobenzamide; (#53), N-(4-Chloro-2-(4-(3,3,3-trifluoropropyl)piperazin-1-yl)phenyl)-4-((4-(dimethylamino)piperidin-1-yl)methyl)-2-fluorobenzamide; (#54), N-(4-Chloro-2-(4-(3,3,3-trifluoropropyl)piperazin-1-yl)phenyl)-2-fluoro-4-((4-(hydroxymethyl)piperidin-1-yl)methyl)benzamide; (#55), 4-((2-Amino-2-oxoethylamino)methyl)-N-(4-chloro-2-(4-(3,3,3-trifluoropropyl)piperazin-1-yl)phenyl)-2-fluorobenzamide; (#56), (S)-1-(4-(4-Chloro-2-(4-(3,3,3-trifluoropropyl)piperazin-1-yl)phenylcarbamoyl)-3-fluorobenzyl)pyrrolidine-2-carboxamide; (#57), N-(4-Chloro-2-(4-(3,3,3-trifluoropropyl)piperazin-1-yl)phenyl)-2-fluoro-4-(((2-hydroxyethyl)(methyl)amino) methyl)benzamide; (#58), N-(4-Chloro-2-(4-(3,3,3-trifluoropropyl)piperazin-1-yl)phenyl)-2-fluoro-4-((2-(2-hydroxyethyl)piperidin-1-yl)methyl)benzamide; (#59), 4-(4-(4-Chloro-2-(4-(3,3,3-trifluoropropyl)piperazin-1-yl)phenylcarbamoyl)-3-fluorobenzyl)piperazine-1-carboxamide; (#60), N-(4-Chloro-2-(4-(3,3,3-trifluoropropyl)piperazin-1-yl)phenyl)-2-fluoro-4-((3-oxopiperazin-1-yl)methyl)benzamide; (#61), N-(4-Chloro-2-(4-(3,3,3-trifluoropropyl)piperazin-1-yl)phenyl)-2-fluoro-4-(((1-methylpiperidin-4-yl)methylamino)methyl)benzamide; (#62), N-(4-Chloro-2-(4-(3,3,3-trifluoropropyl)piperazin-1-yl)phenyl)-2-fluoro-4-((2-hydroxyethylamino)methyl)benzamide; (#63), 4-((3-(1H-Imidazol-1-yl)propylamino)methyl)-N-(4-chloro-2-(4-(3,3,3-trifluoropropyl)piperazin-1-yl)phenyl)-2-fluorobenzamide; (#64), N-(4-Chloro-2-(4-(3,3,3-trifluoropropyl)piperazin-1-yl)phenyl)-2-fluoro-4-((4-methylpyridin-3-ylamino)methyl)benzamide; (#65), N-(4-Chloro-2-(4-(3,3,3-trifluoropropyl)piperazin-1-yl)phenyl)-4-((cyanomethylamino)methyl)-2-fluorobenzamide; (#66), N-(4-Chloro-2-(4-(3,3,3-trifluoropropyl)piperazin-1-yl)phenyl)-4-((4-(2-cyclohexylethyl)piperazin-1-yl)methyl)-2-fluorobenzamide; (#67), (S)-4-((1-Amino-1-oxopropan-2-ylamino)methyl)-N-(4-chloro-2-(4-(3,3,3-trifluoropropyl)piperazin-1-yl)phenyl)-2-fluorobenzamide; (#68), N-(4-Chloro-2-(4-(3,3,3-trifluoropropyl)piperazin-1-yl)phenyl)-2-fluoro-4-((1,1-dioxo-tetrahydrothiophen-3-ylamino)methyl)benzamide; (#69), (R)—N-(4-Chloro-2-(4-(3,3,3-trifluoropropyl)piperazin-1-yl)phenyl)-2-fluoro-4-((1-hydroxy-4-methylpentan-2-ylamino)methyl)benzamide; (#70), 4-(((2-(1H-Imidazol-5-yl)ethyl)(methyl)amino)methyl)-N-(4-chloro-2-(4-(3,3,3-trifluoropropyl)piperazin-1-yl)phenyl)-2-fluorobenzamide; (#71), N-(4-Chloro-2-(4-(3,3,3-trifluoropropyl)piperazin-1-yl)phenyl)-2-fluoro-4-(((1-methyl-1H-imidazol-5-yl)methylamino)methyl)benzamide; (#72), 4-(2,7-Diazaspiro[4.4]nonan-2-ylmethyl)-N-(4-chloro-2-(4-(3,3,3-trifluoropropyl)piperazin-1-yl)phenyl)-2-fluorobenzamide; (#73), 4-(((1S,2R)-2-Carbamoylcyclohexylamino)methyl)-N-(4-chloro-2-(4-(3,3,3-trifluoropropyl)piperazin-1-yl)phenyl)-2-fluorobenzamide; (#74), (S)-Methyl 2-(4-(4-chloro-2-(4-(3,3,3-trifluoropropyl)piperazin-1-yl)phenylcarbamoyl)-3-fluorobenzylamino)-3-hydroxypropanoate; (#75), N-(4-Chloro-2-(4-(3,3,3-trifluoropropyl)piperazin-1-yl)phenyl)-4-((1,3-dihydroxypropan-2-ylamino)methyl)-2-fluorobenzamide; (#76), (S)-4-((1-Amino-3-hydroxy-1-oxopropan-2-ylamino)methyl)-N-(4-chloro-2-(4-(3,3,3-trifluoropropyl)piperazin-1-yl)phenyl)-2-fluorobenzamide; (#77), N-(4-Chloro-2-(4-(3,3,3-trifluoropropyl)piperazin-1-yl)phenyl)-2-fluoro-4-((3-(methylsulfonyl)pyrrolidin-1-yl)methyl)benzamide; (#78), N-(4-Chloro-2-(4-(3,3,3-trifluoropropyl)piperazin-1-yl)phenyl)-2-fluoro-4-(((1S,2S)-2-hydroxycyclohexylamino)methyl)benzamide; (#79), N-(4-Chloro-2-(4-(3,3,3-trifluoropropyl)piperazin-1-yl)phenyl)-2-fluoro-4-((2-oxoazepan-3-ylamino)methyl)benzamide; (#80), N-(4-Chloro-2-(4-(3,3,3-trifluoropropyl)piperazin-1-yl)phenyl)-2-fluoro-4-(((2-hydroxyethyl)(isopropyl)amino)methyl)benzamide; (#81), N-(4-Chloro-2-(4-(3,3,3-trifluoropropyl)piperazin-1-yl)phenyl)-2-fluoro-4-((2-(2-oxoimidazolidin-1-yl)ethylamino)methyl)benzamide; (#82), N-(4-Chloro-2-(4-(3,3,3-trifluoropropyl)piperazin-1-yl)phenyl)-4-((6,7-dihydro-1H-imidazo[4,5-c]pyridin-5(4H)-yl)methyl)-2-fluorobenzamide; (#83), N-(4-Chloro-2-(4-(3,3,3-trifluoropropyl)piperazin-1-yl)phenyl)-2-fluoro-4-((2-(hydroxymethyl)piperidin-1-yl)methyl)benzamide; (#84), (S)-4-((3-Aminopyrrolidin-1-yl)methyl)-N-(4-chloro-2-(4-(3,3,3-trifluoropropyl)piperazin-1-yl)phenyl)-2-fluorobenzamide; (#85), (R)-4-((3-Aminopyrrolidin-1-yl)methyl)-N-(4-chloro-2-(4-(3,3,3-trifluoropropyl)piperazin-1-yl)phenyl)-2-fluorobenzamide; (#86), (S)—N-(4-Chloro-2-(4-(3,3,3-trifluoropropyl)piperazin-1-yl)phenyl)-2-fluoro-4-((pyrrolidin-2-ylmethylamino)methyl)benzamide; (#87), (S)—N-(4-Chloro-2-(4-(3,3,3-trifluoropropyl)piperazin-1-yl)phenyl)-2-fluoro-4-((pyrrolidin-3-ylamino)methyl)benzamide; (#88), (R)—N-(4-Chloro-2-(4-(3,3,3-trifluoropropyl)piperazin-1-yl)phenyl)-2-fluoro-4-((2-methylpiperazin-1-yl)methyl)benzamide; (#89), (S)-4-((3-Aminopiperidin-1-yl)methyl)-N-(4-chloro-2-(4-(3,3,3-trifluoropropyl)piperazin-1-yl)phenyl)-2-fluorobenzamide; (#90), (R)-4-((3-Aminopiperidin-1-yl)methyl)-N-(4-chloro-2-(4-(3,3,3-trifluoropropyl)piperazin-1-yl)phenyl)-2-fluorobenzamide; (#91), (S)—N-(4-Chloro-2-(4-(3,3,3-trifluoropropyl)piperazin-1-yl)phenyl)-2-fluoro-4-((piperidin-3-ylamino)methyl)benzamide; (#92), 4-((4-Aminopiperidin-1-yl)methyl)-N-(4-chloro-2-(4-(3,3,3-trifluoropropyl)piperazin-1-yl)phenyl)-2-fluorobenzamide; (#93), N-(4-Chloro-2-(4-(3,3,3-trifluoropropyl)piperazin-1-yl)phenyl)-2-fluoro-4-((piperidin-4-ylamino)methyl)benzamide; (#94), 1-(4-(4-Chloro-2-(4-(3,3,3-trifluoropropyl)piperazin-1-yl)phenylcarbamoyl)-3-fluorobenzyl)piperidine-2-carboxamide; (#95), N-(4-Chloro-2-(4-(3,3,3-trifluoropropyl)piperazin-1-yl)phenyl)-4-((5,6-dihydropyrimidin-1(4H)-yl)methyl)-2-fluorobenzamide; (#96), (4R)-Methyl 1-(4-(4-chloro-2-(4-(3,3,3-trifluoropropyl)piperazin-1-yl)phenylcarbamoyl)-3-fluorobenzyl)-4-hydroxypyrrolidine-2-carboxylate; (#97), N-(4-Chloro-2-(4-(3,3,3-trifluoropropyl)piperazin-1-yl)phenyl)-2-fluoro-4-((2-hydroxypropylamino)methyl)benzamide; (#98), N-(4-Chloro-2-(4-(3,3,3-trifluoropropyl)piperazin-1-yl)phenyl)-2-fluoro-4-((4-hydroxypiperidin-1-yl)methyl)benzamide; (#99), N-(4-Chloro-2-(4-(3,3,3-trifluoropropyl)piperazin-1-yl)phenyl)-2-fluoro-4-((4-(2-(pyridin-2-yl)ethyl)piperazin-1-yl)methyl)benzamide; (#100), N-(4-Chloro-2-(4-(3,3,3-trifluoropropyl)piperazin-1-yl)phenyl)-4-((ethyl(2-hydroxypyridin-3-yl)amino)methyl)-2-fluorobenzamide; (#101), N-(4-Chloro-2-(4-(3,3,3-trifluoropropyl)piperazin-1-yl)phenyl)-2-fluoro-4-((2-(4-methylpiperazin-1-yl)ethylamino)methyl)benzamide; (#102), (S)—N-(4-Chloro-2-(4-(3,3,3-trifluoropropyl)piperazin-1-yl)phenyl)-2-fluoro-4-((1-hydroxypropan-2-ylamino)methyl)benzamide; (#103), N-(4-Chloro-2-(4-(3,3,3-trifluoropropyl)piperazin-1-yl)phenyl)-4-((1,3-dihydroxy-2-(hydroxymethyl)propan-2-ylamino)methyl)-2-fluorobenzamide; (#104), N-(4-Chloro-2-(4-(3,3,3-trifluoropropyl)piperazin-1-yl)phenyl)-2-fluoro-4-((3-hydroxypiperidin-1-yl)methyl)benzamide; (#105), 4-((2-Acetamidoethylamino)methyl)-N-(4-chloro-2-(4-(3,3,3-trifluoropropyl)piperazin-1-yl)phenyl)-2-fluorobenzamide; (#106), (R)—N-(4-Chloro-2-(4-(3,3,3-trifluoropropyl)piperazin-1-yl)phenyl)-2-fluoro-4-((1-hydroxybutan-2-ylamino)methyl)benzamide; (#107), 1-(4-(4-Chloro-2-(4-(3,3,3-trifluoropropyl)piperazin-1-yl)phenylcarbamoyl)-3-fluorobenzyl)-N,N-diethylpiperidine-3-carboxamide; (#108), N-(4-Chloro-2-(4-(3,3,3-trifluoropropyl)piperazin-1-yl)phenyl)-2-fluoro-4-((2,3,4,6,7,8-hexahydro-1H-pyrimido[1,2-a]pyrimidin-1-yl)methyl)benzamide; (#109), N-(4-Chloro-2-(4-(3,3,3-trifluoropropyl)piperazin-1-yl)phenyl)-2-fluoro-4-((2-(1-methylpyrrolidin-2-yl)ethylamino)methyl)benzamide; (#110), N-(4-Chloro-2-(4-(3,3,3-trifluoropropyl)piperazin-1-yl)phenyl)-4-(((2-(dimethylamino)ethyl)(methyl)amino)methyl)-2-fluorobenzamide; (#111), N-(4-Chloro-2-(4-(3,3,3-trifluoropropyl)piperazin-1-yl)phenyl)-4-((4-cyclopentylpiperazin-1-yl)methyl)-2-fluorobenzamide; (#112), 4-((1,4-Oxazepan-4-yl)methyl)-N-(4-chloro-2-(4-(3,3,3-trifluoropropyl)piperazin-1-yl)phenyl)-2-fluorobenzamide; (#113), (S)—N-(4-Chloro-2-(4-(3,3,3-trifluoropropyl)piperazin-1-yl)phenyl)-2-fluoro-4-((2-(pyrrolidin-1-ylmethyl)pyrrolidin-1-yl)methyl)benzamide; (#114), N-(4-Chloro-2-(4-(3,3,3-trifluoropropyl)piperazin-1-yl)phenyl)-2-fluoro-4-((2-morpholinoethylamino)methyl)benzamide; (#115), N-(4-Chloro-2-(4-(3,3,3-trifluoropropyl)piperazin-1-yl)phenyl)-2-fluoro-4-((4-morpholinopiperidin-1-yl)methyl)benzamide; (#116), N-(4-Chloro-2-(4-(3,3,3-trifluoropropyl)piperazin-1-yl)phenyl)-4-((2-(dimethylamino)ethylamino)methyl)-2-fluorobenzamide; (#117), N-(4-Chloro-2-(4-(3,3,3-trifluoropropyl)piperazin-1-yl)phenyl)-4-((1-ethyl-2-oxoazepan-3-ylamino)methyl)-2-fluorobenzamide; (#118), N-(4-Chloro-2-(4-(3,3,3-trifluoropropyl)piperazin-1-yl)phenyl)-4-((4-(cyclohexylmethyl)piperazin-1-yl)methyl)-2-fluorobenzamide; (#119), N-(4-Chloro-2-(4-(3,3,3-trifluoropropyl)piperazin-1-yl)phenyl)-2-fluoro-4-((4-oxopiperidin-1-yl)methyl)benzamide; (#120), N-(4-Chloro-2-(4-(3,3,3-trifluoropropyl)piperazin-1-yl)phenyl)-4-(((3-(dimethylamino)tetrahydrothiophen-3-yl)methylamino)methyl)-2-fluorobenzamide; (#121), N-(4-Chloro-2-(4-(3,3,3-trifluoropropyl)piperazin-1-yl)phenyl)-4-(((2-(diethylamino)ethyl)(methyl)amino)methyl)-2-fluorobenzamide; (#122), N-(4-Chloro-2-(4-(3,3,3-trifluoropropyl)piperazin-1-yl)phenyl)-2-fluoro-4-(((2S,3S)-1-hydroxy-3-methylpentan-2-ylamino)methyl)benzamide; (#123), 4-((5-Aminopentylamino)methyl)-N-(4-chloro-2-(4-(3,3,3-trifluoropropyl)piperazin-1-yl)phenyl)-2-fluorobenzamide; (#124), 4-((3-Amino-3-oxopropanimidamido)methyl)-N-(4-chloro-2-(4-(3,3,3-trifluoropropyl)piperazin-1-yl)phenyl)-2-fluorobenzamide; (#125), 4-((4-Acetylpiperazin-1-yl)methyl)-N-(4-chloro-2-(4-(3,3,3-trifluoropropyl)piperazin-1-yl)phenyl)-2-fluorobenzamide; (#126), 4-(1,4′-Bipiperidin-1′-ylmethyl)-N-(4-chloro-2-(4-(3,3,3-trifluoropropyl)piperazin-1-yl)phenyl)-2-fluorobenzamide; (#127), N-(4-Chloro-2-(4-(3,3,3-trifluoropropyl)piperazin-1-yl)phenyl)-2-fluoro-4-(((1-hydroxycyclohexyl)methylamino)methyl)benzamide; (#128), (S)—N-(4-Chloro-2-(4-(3,3,3-trifluoropropyl)piperazin-1-yl)phenyl)-2-fluoro-4-((2-oxoazepan-3-ylamino)methyl)benzamide; (#129), Ethyl 1-(4-(4-chloro-2-(4-(3,3,3-trifluoropropyl)piperazin-1-yl)phenylcarbamoyl)-3-fluorobenzyl)piperidine-4-carboxylate; (#130), (R)—N-(4-Chloro-2-(4-(3,3,3-trifluoropropyl)piperazin-1-yl)phenyl)-2-fluoro-4-((2-hydroxypropylamino)methyl)benzamide; (#131), N-(4-Chloro-2-(4-(3,3,3-trifluoropropyl)piperazin-1-yl)phenyl)-2-fluoro-4-((2-(hydroxymethyl)morpholino) methyl)benzamide; (#132), (R)—N-(4-Chloro-2-(4-(3,3,3-trifluoropropyl)piperazin-1-yl)phenyl)-2-fluoro-4-((2-(2-(hydroxymethyl)pyrrolidin-1-yl)ethylamino)methyl)benzamide; (#133), N-(4-Chloro-2-(4-(3,3,3-trifluoropropyl)piperazin-1-yl)phenyl)-2-fluoro-4-((2-methyl-2-(piperidin-1-yl)propylamino)methyl)benzamide; (#134), (S)—N-(4-Chloro-2-(4-(3,3,3-trifluoropropyl)piperazin-1-yl)phenyl)-2-fluoro-4-((2-(hydroxymethyl)pyrrolidin-1-yl)methyl)benzamide; (#135), N-(4-Chloro-2-(4-(3,3,3-trifluoropropyl)piperazin-1-yl)phenyl)-2-fluoro-4-((3-hydroxyazetidin-1-yl)methyl)benzamide; (#136), (S)—N-(4-Chloro-2-(4-(3,3,3-trifluoropropyl)piperazin-1-yl)phenyl)-2-fluoro-4-((2-hydroxypropylamino)methyl)benzamide; (#137), N-(4-Chloro-2-(4-(3,3,3-trifluoropropyl)piperazin-1-yl)phenyl)-2-fluoro-4-((4-hydroxy-1,4′-bipiperidin-1′-yl)methyl)benzamide; (#138), N-(4-Chloro-2-(4-(3,3,3-trifluoropropyl)piperazin-1-yl)phenyl)-4-((2-(diethylamino)ethylamino)methyl)-2-fluorobenzamide; (#139), 4-(Benzyl(2-hydroxyethyl)amino)methyl)-N-(4-chloro-2-(4-(3,3,3-trifluoropropyl)piperazin-1-yl)phenyl)-2-fluorobenzamide; (#140), N-(4-Chloro-2-(4-(3,3,3-trifluoropropyl)piperazin-1-yl)phenyl)-2-fluoro-4-((2-(methylsulfinyl)ethylamino)methyl)benzamide; (#141), N-(4-Chloro-2-(4-(3,3,3-trifluoropropyl)piperazin-1-yl)phenyl)-2-fluoro-4-((3-hydroxypyrrolidin-1-yl)methyl)benzamide; (#142), 4-(Bis(2-cyanoethyl)amino)methyl)-N-(4-chloro-2-(4-(3,3,3-trifluoropropyl)piperazin-1-yl)phenyl)-2-fluorobenzamide; (#143), 4-((2-(Azepan-1-yl)ethylamino)methyl)-N-(4-chloro-2-(4-(3,3,3-trifluoropropyl)piperazin-1-yl)phenyl)-2-fluorobenzamide; (#144), 4-(Bis(2-hydroxyethyl)amino)methyl)-N-(4-chloro-2-(4-(3,3,3-trifluoropropyl)piperazin-1-yl)phenyl)-2-fluorobenzamide; (#145), N-(4-Chloro-2-(4-(3,3,3-trifluoropropyl)piperazin-1-yl)phenyl)-2-fluoro-4-((3-hydroxybutylamino)methyl)benzamide; (#146), 4-((tert-butyl(2-hydroxyethyl)amino)methyl)-N-(4-chloro-2-(4-(3,3,3-trifluoropropyl)piperazin-1-yl)phenyl)-2-fluorobenzamide; (#147), N-(4-Chloro-2-(4-(3,3,3-trifluoropropyl)piperazin-1-yl)phenyl)-2-fluoro-4-((3-(hydroxymethyl)piperidin-1-yl)methyl)benzamide; (#148), 4-(2,5-Diazabicyclo[2.2.1]heptan-2-ylmethyl)-N-(4-chloro-2-(4-(3,3,3-trifluoropropyl)piperazin-1-yl)phenyl)-2-fluorobenzamide; (#149), (S)-4-((1-Amino-3-methyl-1-oxobutan-2-ylamino)methyl)-N-(4-chloro-2-(4-(3,3,3-trifluoropropyl)piperazin-1-yl)phenyl)-2-fluorobenzamide; (#150), 4-((2-(2-(2-Aminoethoxy)ethoxy)ethylamino)methyl)-N-(4-chloro-2-(4-(3,3,3-trifluoropropyl)piperazin-1-yl)phenyl)-2-fluorobenzamide; (#151), (S)—N-(4-Chloro-2-(4-(3,3,3-trifluoropropyl)piperazin-1-yl)phenyl)-2-fluoro-4-((2-(2-(hydroxymethyl)pyrrolidin-1-yl)ethylamino)methyl)benzamide; (#152), (R)-Methyl 2-(4-(4-chloro-2-(4-(3,3,3-trifluoropropyl)piperazin-1-yl)phenylcarbamoyl)-3-fluorobenzylamino)-3-hydroxypropanoate; (#153), N-(4-Chloro-2-(4-(3,3,3-trifluoropropyl)piperazin-1-yl)phenyl)-4-(((2R,3S)-1,3-dihydroxybutan-2-ylamino)methyl)-2-fluorobenzamide; (#154), N-(4-Chloro-2-(4-(3,3,3-trifluoropropyl)piperazin-1-yl)phenyl)-2-fluoro-4-((2-morpholino-2-oxoethylamino)methyl)benzamide; (#155), N-(4-Chloro-2-(4-(3,3,3-trifluoropropyl)piperazin-1-yl)phenyl)-4-((2-(dimethylamino)-2-(pyridin-3-yl)ethylamino)methyl)-2-fluorobenzamide; (#156), N-(4-Chloro-2-(4-(3,3,3-trifluoropropyl)piperazin-1-yl)phenyl)-2-fluoro-4-((5-oxo-1,4-diazepan-1-yl)methyl)benzamide; (#157), N-(4-Chloro-2-(4-(3,3,3-trifluoropropyl)piperazin-1-yl)phenyl)-2-fluoro-4-((hexahydropyrrolo[1,2-a]pyrazin-2(1H)-yl)methyl)benzamide; (#158), N-(4-Chloro-2-(4-(3,3,3-trifluoropropyl)piperazin-1-yl)phenyl)-2-fluoro-4-((methyl(2-morpholino-2-oxoethyl)amino)methyl)benzamide; (#159), N-(4-Chloro-2-(4-(3,3,3-trifluoropropyl)piperazin-1-yl)phenyl)-2-fluoro-4-((2-(pyrrolidin-1-yl)ethylamino)methyl)benzamide; (#160), (S)-4-((1-Amino-3-hydroxy-1-oxopropan-2-ylamino)methyl)-N-(4,5-dichloro-2-(4-(3,3,3-trifluoropropyl)piperazin-1-yl)phenyl)-2-fluorobenzamide; (#161), N-(4,5-Dichloro-2-(4-(3,3,3-trifluoropropyl)piperazin-1-yl)phenyl)-2-fluoro-4-((1,1-dioxo-thiomorpholin-4-yl)methyl)benzamide; (#162), (S)-1-(4-(4,5-Dichloro-2-(4-(3,3,3-trifluoropropyl)piperazin-1-yl)phenylcarbamoyl)-3-fluorobenzyl)pyrrolidine-2-carboxamide; (#163), N-(4,5-Dichloro-2-(4-(3,3,3-trifluoropropyl)piperazin-1-yl)phenyl)-2-fluoro-4-((3-oxopiperazin-1-yl)methyl)benzamide; (#164), N-(4,5-Dichloro-2-(4-(3,3,3-trifluoropropyl)piperazin-1-yl)phenyl)-2-fluoro-4-((1,1-dioxo-tetrahydrothiophen-3-ylamino)methyl)benzamide; (#165), N-(4-Chloro-2-(4-(3,3,3-trifluoropropyl)piperazin-1-yl)phenyl)-2,3-difluoro-4-((4-(hydroxymethyl)piperidin-1-yl)methyl)benzamide; (#166), N-(4-Chloro-2-(4-(3,3,3-trifluoropropyl)piperazin-1-yl)phenyl)-2,3-difluoro-4-((4-hydroxypiperidin-1-yl)methyl)benzamide; (#167), N-(4-Chloro-2-(4-(3,3,3-trifluoropropyl)piperazin-1-yl)phenyl)-2,3-difluoro-4-((3-oxopiperazin-1-yl)methyl)benzamide; (#168), N-(4-Chloro-2-(4-(3,3,3-trifluoropropyl)piperazin-1-yl)phenyl)-2,3-difluoro-4-((1,1-dioxo-thiomorpholin-4-yl)methyl)benzamide; (#169), (S)-1-(4-(4-Chloro-2-(4-(3,3,3-trifluoropropyl)piperazin-1-yl)phenylcarbamoyl)-2,3-difluorobenzyl)pyrrolidine-2-carboxamide; (#170), (S)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide; (#171), N-(4-Chloro-2-(4-(3,3,3-trifluoropropyl)piperazin-1-yl)phenyl)-2,3-difluoro-4-((1,1-dioxo-tetrahydrothiophen-3-ylamino)methyl)benzamide; (#172), N-(4-Chloro-2-(4-(3,3,3-trifluoropropyl)piperazin-1-yl)phenyl)-2,3-difluoro-4-(((2-hydroxyethyl)(methyl)amino)methyl)benzamide; (#173), N-(4-Chloro-2-(4-(3,3,3-trifluoropropyl)piperazin-1-yl)phenyl)-2,3-difluoro-4-((2-(2-hydroxyethyl)piperidin-1-yl)methyl)benzamide; (#174), (R)—N-(4-Chloro-2-(4-(3,3,3-trifluoropropyl)piperazin-1-yl)phenyl)-2,3-difluoro-4-((2-(hydroxymethyl)pyrrolidin-1-yl)methyl)benzamide; (#175), N-(4-Chloro-2-(4-(3,3,3-trifluoropropyl)piperazin-1-yl)phenyl)-2,3-difluoro-4-((4-(2-hydroxyethyl)piperazin-1-yl)methyl)benzamide; (#176), N-(4-Chloro-2-(4-(3,3,3-trifluoropropyl)piperazin-1-yl)phenyl)-2,3-difluoro-4-(((1R,2S)-2-hydroxycyclohexylamino) methyl)benzamide; (#177), (S)—N-(4-Chloro-2-(4-(3,3,3-trifluoropropyl)piperazin-1-yl)phenyl)-2,3-difluoro-4-((3-hydroxypyrrolidin-1-yl)methyl)benzamide; (#178), 4-(Bis(2-hydroxyethyl)amino)methyl)-N-(4-chloro-2-(4-(3,3,3-trifluoropropyl)piperazin-1-yl)phenyl)-2,3-difluorobenzamide; (#179), N-(4-Chloro-2-(4-(3,3,3-trifluoropropyl)piperazin-1-yl)phenyl)-2,3-difluoro-4-((3-hydroxyazetidin-1-yl)methyl)benzamide; (#180), N-(4-Chloro-2-(4-(3,3,3-trifluoropropyl)piperazin-1-yl)phenyl)-2,3-difluoro-4-((2-(hydroxymethyl)morpholino) methyl)benzamide; (#181), (R)—N-(4-Chloro-2-(4-(3,3,3-trifluoropropyl)piperazin-1-yl)phenyl)-2,3-difluoro-4-((3-hydroxypiperidin-1-yl)methyl)benzamide; (#182), (S)—N-(4-Chloro-2-(4-(3,3,3-trifluoropropyl)piperazin-1-yl)phenyl)-2,3-difluoro-4-((3-hydroxypiperidin-1-yl)methyl)benzamide; (#183), N-(4-Chloro-2-(4-(3,3,3-trifluoropropyl)piperazin-1-yl)phenyl)-2,3-difluoro-4-((4-(2-hydroxyethyl)piperidin-1-yl)methyl)benzamide; (#184), N-(4-Chloro-2-(4-(3,3,3-trifluoropropyl)piperazin-1-yl)phenyl)-2,3-difluoro-4-(piperazin-1-ylmethyl)benzamide; (#185), (S)-4-((1-Amino-3-hydroxy-1-oxopropan-2-ylamino)methyl)-N-(4,5-dichloro-2-(4-(3,3,3-trifluoropropyl)piperazin-1-yl)phenyl)-2,3-difluorobenzamide; (#186), N-(4,5-Dichloro-2-(4-(3,3,3-trifluoropropyl)piperazin-1-yl)phenyl)-2,3-difluoro-4-((1,1-dioxo-thiomorpholin-4-yl)methyl)benzamide; (#187), (S)-1-(4-(4,5-Dichloro-2-(4-(3,3,3-trifluoropropyl)piperazin-1-yl)phenylcarbamoyl)-2,3-difluorobenzyl)pyrrolidine-2-carboxamide; (#188), N-(4,5-Dichloro-2-(4-(3,3,3-trifluoropropyl)piperazin-1-yl)phenyl)-2,3-difluoro-4-((3-oxopiperazin-1-yl)methyl)benzamide; (#189), N-(4,5-Dichloro-2-(4-(3,3,3-trifluoropropyl)piperazin-1-yl)phenyl)-2,3-difluoro-4-((1,1-dioxo-tetrahydrothiophen-3-ylamino)methyl)benzamide; (#190), N-(4,5-Dichloro-2-(4-(3,3,3-trifluoropropyl)piperazin-1-yl)phenyl)-2,3-difluoro-4-((4-(2-hydroxyethyl)piperidin-1-yl)methyl)benzamide; (#191), N-(4,5-Dichloro-2-(4-(3,3,3-trifluoropropyl)piperazin-1-yl)phenyl)-2,3-difluoro-4-((2-(hydroxymethyl)morpholino) methyl)benzamide; (#192), N-(4,5-Dichloro-2-(4-(3,3,3-trifluoropropyl)piperazin-1-yl)phenyl)-2,3-difluoro-4-((3-hydroxyazetidin-1-yl)methyl)benzamide; (#193), 4-Amino-N-(4-chloro-2-(4-(3,3,3-trifluoropropyl)piperazin-1-yl)phenyl)-2-fluorobenzamide; (#194), 4-Amino-5-chloro-N-(4-chloro-2-(4-(3,3,3-trifluoropropyl)piperazin-1-yl)phenyl)-2-fluorobenzamide; (#195), (R)-4-((1-Amino-3-hydroxy-1-oxopropan-2-ylamino)methyl)-N-(4-chloro-2-(4-(3,3,3-trifluoropropyl)piperazin-1-yl)phenyl)-2-fluorobenzamide; (#196), (R)-Methyl 3-amino-2-(4-(4-chloro-2-(4-(3,3,3-trifluoropropyl)piperazin-1-yl)phenylcarbamoyl)-3-fluorobenzylamino)propanoate; (#197), (R)-Methyl 4-amino-2-(4-(4-chloro-2-(4-(3,3,3-trifluoropropyl)piperazin-1-yl)phenylcarbamoyl)-3-fluorobenzylamino)butanoate; (#198), (S)-Methyl 3-amino-2-(4-(4-chloro-2-(4-(3,3,3-trifluoropropyl)piperazin-1-yl)phenylcarbamoyl)-3-fluorobenzylamino)propanoate; (#199), (R)-2-(4-(4-Chloro-2-(4-(3,3,3-trifluoropropyl)piperazin-1-yl)phenylcarbamoyl)-3-fluorobenzylamino)-3-hydroxypropanoic acid; (#200), 4-(((2H-Tetrazol-5-yl)methylamino)methyl)-N-(4-chloro-2-(4-(3,3,3-trifluoropropyl)piperazin-1-yl)phenyl)-2,3-difluorobenzamide; (#201), N-(4-Chloro-2-(4-(3,3,3-trifluoropropyl)piperazin-1-yl)phenyl)-2,3-difluoro-4-(((3-oxo-2,3-dihydroisoxazol-5-yl)methylamino)methyl)benzamide; (#202), 2-(4-(4-Chloro-2-(4-(3,3,3-trifluoropropyl)piperazin-1-yl)phenylcarbamoyl)-2,3-difluorobenzylamino)acetic acid; (#203), 4-(4-(4-Chloro-2-(4-(3,3,3-trifluoropropyl)piperazin-1-yl)phenylcarbamoyl)-2,3-difluorobenzylamino)butanoic acid; (#204), (S)-2-(4-(4-Chloro-2-(4-(3,3,3-trifluoropropyl)piperazin-1-yl)phenylcarbamoyl)-2,3-difluorobenzylamino)-3-hydroxypropanoic acid; (#205), 4-Amino-3-chloro-N-(4-chloro-2-(4-(3,3,3-trifluoropropyl)piperazin-1-yl)phenyl)-2-fluorobenzamide; (#206), 2-(1-(4-(4-Chloro-2-(4-(3,3,3-trifluoropropyl)piperazin-1-yl)phenylcarbamoyl)-2,3-difluorobenzyl)pyrrolidin-3-yl)acetic acid; (#207), 3-(4-(4-Chloro-2-(4-(3,3,3-trifluoropropyl)piperazin-1-yl)phenylcarbamoyl)-2,3-difluorobenzylamino)propanoic acid; (#208), (S)-4-Amino-2-(4-(4-chloro-2-(4-(3,3,3-trifluoropropyl)piperazin-1-yl)phenylcarbamoyl)-2,3-difluorobenzylamino)-4-oxobutanoic acid; (#209), (S)-2-(4-(4-Chloro-2-(4-(3,3,3-trifluoropropyl)piperazin-1-yl)phenylcarbamoyl)-2,3-difluorobenzylamino)-4-methylpentanoic acid; (#210), 2-((4-(4-Chloro-2-(4-(3,3,3-trifluoropropyl)piperazin-1-yl)phenylcarbamoyl)-2,3-difluorobenzyl)(methyl)amino)acetic acid; (#211), (S)-2-(4-(4-Chloro-2-(4-(3,3,3-trifluoropropyl)piperazin-1-yl)phenylcarbamoyl)-2,3-difluorobenzylamino)pentanedioic acid; (#212), 2-(4-(4-Chloro-2-(4-(3,3,3-trifluoropropyl)piperazin-1-yl)phenylcarbamoyl)-2,3-difluorobenzylamino)-2-methylpropanoic acid; (#213), (S)-4-(4-(4-Chloro-2-(4-(3,3,3-trifluoropropyl)piperazin-1-yl)phenylcarbamoyl)-2,3-difluorobenzylamino)-5-methoxy-5-oxopentanoic acid; (#214), (S)-3-(4-(4-Chloro-2-(4-(3,3,3-trifluoropropyl)piperazin-1-yl)phenylcarbamoyl)-2,3-difluorobenzylamino)-4-methoxy-4-oxobutanoic acid; (#215), N-(4-Chloro-2-(4-(3,3,3-trifluoropropyl)piperazin-1-yl)phenyl)-2,3-difluoro-4-((piperidin-4-ylamino)methyl)benzamide; (#216), 4-(2,7-Diazaspiro[3.5]nonan-2-ylmethyl)-N-(4-chloro-2-(4-(3,3,3-trifluoropropyl)piperazin-1-yl)phenyl)-2,3-difluorobenzamide; (#217), (R)—N-(4-Chloro-2-(4-(3,3,3-trifluoropropyl)piperazin-1-yl)phenyl)-2,3-difluoro-4-((piperidin-3-ylamino)methyl)benzamide; (#218), (R)—N-(4-Chloro-2-(4-(3,3,3-trifluoropropyl)piperazin-1-yl)phenyl)-2,3-difluoro-4-((2-(hydroxymethyl)morpholino) methyl)benzamide; (#219), 2-(4-(tert-Butoxycarbonyl)-1-(4-(4-chloro-2-(4-(3,3,3-trifluoropropyl)piperazin-1-yl)phenylcarbamoyl)-2,3-difluorobenzyl)piperazin-2-yl)acetic acid; (#220), 4-(tert-Butoxycarbonyl)-1-(4-(4-chloro-2-(4-(3,3,3-trifluoropropyl)piperazin-1-yl)phenylcarbamoyl)-2,3-difluorobenzyl)piperazine-2-carboxylic acid; (#221), (S)-4-(tert-Butoxycarbonylamino)-2-(4-(4-chloro-2-(4-(3,3,3-trifluoropropyl)piperazin-1-yl)phenylcarbamoyl)-2,3-difluorobenzylamino)butanoic acid; (#222), (R)-3-(tert-Butoxycarbonylamino)-2-(4-(4-chloro-2-(4-(3,3,3-trifluoropropyl)piperazin-1-yl)phenylcarbamoyl)-2,3-difluorobenzylamino)propanoic acid; (#223), (S)-2-(4-(tert-Butoxycarbonyl)-1-(4-(4-chloro-2-(4-(3,3,3-trifluoropropyl)piperazin-1-yl)phenylcarbamoyl)-2,3-difluorobenzyl)piperazin-2-yl)acetic acid; (#224), 1-(4-(4-Chloro-2-(4-(3,3,3-trifluoropropyl)piperazin-1-yl)phenylcarbamoyl)-2,3-difluorobenzyl)piperidine-4-carboxylic acid; (#225), 2-(4-(4-(4-Chloro-2-(4-(3,3,3-trifluoropropyl)piperazin-1-yl)phenylcarbamoyl)-2,3-difluorobenzyl)morpholin-2-yl)acetic acid; (#226), 2-(1-(4-(4-Chloro-2-(4-(3,3,3-trifluoropropyl)piperazin-1-yl)phenylcarbamoyl)-2,3-difluorobenzyl)piperazin-2-yl)acetic acid; (#227), 1-(4-(4-Chloro-2-(4-(3,3,3-trifluoropropyl)piperazin-1-yl)phenylcarbamoyl)-2,3-difluorobenzyl)piperazine-2-carboxylic acid; (#228), (R)-2-(4-(4-Chloro-2-(4-(3,3,3-trifluoropropyl)piperazin-1-yl)phenylcarbamoyl)-2,3-difluorobenzylamino)-3-hydroxypropanoic acid; (#229), (S)-4-Amino-2-(4-(4-chloro-2-(4-(3,3,3-trifluoropropyl)piperazin-1-yl)phenylcarbamoyl)-2,3-difluorobenzylamino)butanoic acid; (#230), (R)-3-Amino-2-(4-(4-chloro-2-(4-(3,3,3-trifluoropropyl)piperazin-1-yl)phenylcarbamoyl)-2,3-difluorobenzylamino)propanoic acid; (#231), (S)-2-(1-(4-(4-Chloro-2-(4-(3,3,3-trifluoropropyl)piperazin-1-yl)phenylcarbamoyl)-2,3-difluorobenzyl)piperazin-2-yl)acetic acid; (#232), 2-(4-(4-(4-Chloro-2-(4-(3,3,3-trifluoropropyl)piperazin-1-yl)phenylcarbamoyl)-2,3-difluorobenzyl)piperazin-1-yl)acetic acid; (#233), 6-(4-(4-Chloro-2-(4-(3,3,3-trifluoropropyl)piperazin-1-yl)phenylcarbamoyl)-2,3-difluorobenzylamino)hexanoic acid; (#234), 7-(4-(4-Chloro-2-(4-(3,3,3-trifluoropropyl)piperazin-1-yl)phenylcarbamoyl)-2,3-difluorobenzyl)-5,6,7,8-tetrahydroimidazo[1,2-a]pyrazine-2-carboxylic acid; (#235), N-(4-Chloro-2-(4-(3,3,3-trifluoropropyl)piperazin-1-yl)phenyl)-4-((2-cyanoethylamino)methyl)-2,3-difluorobenzamide; (#236), 4-Amino-N-(4-chloro-2-(4-(3,3,3-trifluoropropyl)piperazin-1-yl)phenyl)-2,3-difluorobenzamide; (#237), 4-(((1S,2R)-2-Carbamoylcyclohexylamino)methyl)-N-(4-chloro-2-(4-(3,3,3-trifluoropropyl)piperazin-1-yl)phenyl)-2,3-difluorobenzamide; (#238), 4-(Aminomethyl)-N-(4-bromo-2-(4-(3,3,3-trifluoropropyl)piperazin-1-yl)phenyl)-2,3-difluorobenzamide; (#239), 1-(4-(4-Chloro-2-(4-(3,3,3-trifluoropropyl)piperazin-1-yl)phenylcarbamoyl)-2,3-difluorobenzyl)piperazine-2-carboxamide; (#240), 1-(4-(4-Chloro-2-(4-(3,3,3-trifluoropropyl)piperazin-1-yl)phenylcarbamoyl)-2,3-difluorobenzyl)-N-methylpiperazine-2-carboxamide; (#241), 1-(4-(4-Chloro-2-(4-(3,3,3-trifluoropropyl)piperazin-1-yl)phenylcarbamoyl)-2,3-difluorobenzyl)-N-(2-hydroxyethyl)piperazine-2-carboxamide; (#242), 1-(4-(4-Chloro-2-(4-(3,3,3-trifluoropropyl)piperazin-1-yl)phenylcarbamoyl)-2,3-difluorobenzyl)-N—((S)-1-hydroxypropan-2-yl)piperazine-2-carboxamide; (#243), 4-(4-(4-Chloro-2-(4-(3,3,3-trifluoropropyl)piperazin-1-yl)phenylcarbamoyl)-2,3-difluorobenzyl)piperazine-1-carboxamide; (#244), 1-(4-(4-Chloro-2-(4-(3,3,3-trifluoropropyl)piperazin-1-yl)phenylcarbamoyl)-2,3-difluorobenzyl)piperidine-3-carboxamide; (#245), 1-(4-(4-Chloro-2-(4-(3,3,3-trifluoropropyl)piperazin-1-yl)phenylcarbamoyl)-2,3-difluorobenzyl)piperidine-4-carboxamide; (#246), (R)-1-(4-(4-Chloro-2-(4-(3,3,3-trifluoropropyl)piperazin-1-yl)phenylcarbamoyl)-2,3-difluorobenzyl)pyrrolidine-2-carboxamide; (#247), (R)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide; (#248), (S)-4-(((1-Amino-3-hydroxy-1-oxopropan-2-yl)(ethyl)amino)methyl)-N-(4-chloro-2-(4-(3,3,3-trifluoropropyl)piperazin-1-yl)phenyl)-2,3-difluorobenzamide; (#249), (S)-3-Amino-2-(4-(4-chloro-2-(4-(3,3,3-trifluoropropyl)piperazin-1-yl)phenylcarbamoyl)-2,3-difluorobenzylamino)-3-oxopropyl dihydrogenphosphate; (#250), (R)-3-Amino-2-(4-(4-chloro-2-(4-(3,3,3-trifluoropropyl)piperazin-1-yl)phenylcarbamoyl)-2,3-difluorobenzylamino)-3-oxopropyl dihydrogenphosphate; (#251), (S)—N-(4-Chloro-2-(4-(3,3,3-trifluoropropyl)piperazin-1-yl)phenyl)-2,3-difluoro-4-((3-(hydroxymethyl)piperidin-1-yl)methyl)benzamide; (#252), (R)—N-(4-Chloro-2-(4-(3,3,3-trifluoropropyl)piperazin-1-yl)phenyl)-2,3-difluoro-4-((3-(hydroxymethyl)piperidin-1-yl)methyl)benzamide; (#253), (S)—N-(4-Chloro-2-(4-(3,3,3-trifluoropropyl)piperazin-1-yl)phenyl)-2,3-difluoro-4-((2-(hydroxymethyl)pyrrolidin-1-yl)methyl)benzamide; (#254), (S)—N-(4-Chloro-2-(4-(3,3,3-trifluoropropyl)piperazin-1-yl)phenyl)-2,3-difluoro-4-((3-(hydroxymethyl)pyrrolidin-1-yl)methyl)benzamide; (#255), 4-(((1S,2R)-2-Carbamoylcyclopentylamino)methyl)-N-(4-chloro-2-(4-(3,3,3-trifluoropropyl)piperazin-1-yl)phenyl)-2,3-difluorobenzamide; (#256), 2-Butyryl-N-(4-chloro-2-(4-(3,3,3-trifluoropropyl)piperazin-1-yl)phenyl)-1,2,3,4-tetrahydroisoquinoline-7-carboxamide; (#257), N-(4-Chloro-2-(4-(3,3,3-trifluoropropyl)piperazin-1-yl)phenyl)-2-(2-ethylbutanoyl)-1,2,3,4-tetrahydroisoquinoline-7-carboxamide; (#258), N-(4-Chloro-2-(4-(3,3,3-trifluoropropyl)piperazin-1-yl)phenyl)-2-(thiophene-2-carbonyl)-1,2,3,4-tetrahydroisoquinoline-7-carboxamide; (#259), N-(4-Chloro-2-(4-(3,3,3-trifluoropropyl)piperazin-1-yl)phenyl)-2-nicotinoyl-1,2,3,4-tetrahydroisoquinoline-7-carboxamide; (#260), N-(4-Chloro-2-(4-(3,3,3-trifluoropropyl)piperazin-1-yl)phenyl)-2-(2-cyclopentylacetyl)-1,2,3,4-tetrahydroisoquinoline-7-carboxamide; (#261), N-(4-(4-Chloro-2-(4-(3,3,3-trifluoropropyl)piperazin-1-yl)phenylcarbamoyl)-2,3-difluorobenzyl)piperidine-4-carboxamide; (#262), 4-(((1R,2S)-2-Aminocyclohexanecarboxamido)methyl)-N-(4-chloro-2-(4-(3,3,3-trifluoropropyl)piperazin-1-yl)phenyl)-2,3-difluorobenzamide; (#263), (S)—N-(4-(4-Chloro-2-(4-(3,3,3-trifluoropropyl)piperazin-1-yl)phenylcarbamoyl)-2,3-difluorobenzyl)piperidine-3-carboxamide; (#264), N-(4-(4-Chloro-2-(4-(3,3,3-trifluoropropyl)piperazin-1-yl)phenylcarbamoyl)-2,3-difluorobenzyl)morpholine-2-carboxamide; (#265), 4-((3-Aminopropanamido)methyl)-N-(4-chloro-2-(4-(3,3,3-trifluoropropyl)piperazin-1-yl)phenyl)-2,3-difluorobenzamide; (#266), 4-((2-Aminoacetamido)methyl)-N-(4-chloro-2-(4-(3,3,3-trifluoropropyl)piperazin-1-yl)phenyl)-2,3-difluorobenzamide; (#267), (2S,4R)—N-(4-(4-Chloro-2-(4-(3,3,3-trifluoropropyl)piperazin-1-yl)phenylcarbamoyl)-2,3-difluorobenzyl)-4-hydroxypyrrolidine-2-carboxamide; (#268), (S)-4-((2-Aminopropanamido)methyl)-N-(4-chloro-2-(4-(3,3,3-trifluoropropyl)piperazin-1-yl)phenyl)-2,3-difluorobenzamide; (#269), (S)-4-(2-Amino-3-hydroxypropanamido)methyl)-N-(4-chloro-2-(4-(3,3,3-trifluoropropyl)piperazin-1-yl)phenyl)-2,3-difluorobenzamide; (#270), N-(4-Chloro-2-(4-(3,3,3-trifluoropropyl)piperazin-1-yl)phenyl)-2,3-difluoro-4-((2-hydroxyacetamido)methyl)benzamide; (#271), (S)—N-(4-(4-Chloro-2-(4-(3,3,3-trifluoropropyl)piperazin-1-yl)phenylcarbamoyl)-2,3-difluorobenzyl)morpholine-2-carboxamide; (#272), (R)—N-(4-(4-Chloro-2-(4-(3,3,3-trifluoropropyl)piperazin-1-yl)phenylcarbamoyl)-2,3-difluorobenzyl)morpholine-2-carboxamide; (#273), (R)—N-(4-(4-Chloro-2-(4-(3,3,3-trifluoropropyl)piperazin-1-yl)phenylcarbamoyl)-2,3-difluorobenzyl)thiomorpholine-3-carboxamide; (#274), (S)—N-(4-(4-Chloro-2-(4-(3,3,3-trifluoropropyl)piperazin-1-yl)phenylcarbamoyl)-2,3-difluorobenzyl)pyrrolidine-2-carboxamide; (#275), (R)-4-(2-Aminopropanamido)methyl)-N-(4-chloro-2-(4-(3,3,3-trifluoropropyl)piperazin-1-yl)phenyl)-2,3-difluorobenzamide; (#276), (2S)—N-(4-(4-Chloro-2-(4-(3,3,3-trifluoropropyl)piperazin-1-yl)phenylcarbamoyl)-2,3-difluorobenzyl)-4-hydroxypyrrolidine-2-carboxamide; (#277), N-(4-Chloro-2-(4-(3,3,3-trifluoropropyl)piperazin-1-yl)phenyl)-2,3-difluoro-4-((2-morpholinoacetamido)methyl)benzamide; (#278), 4-((2-(1H-Tetrazol-5-yl)acetamido)methyl)-N-(4-chloro-2-(4-(3,3,3-trifluoropropyl)piperazin-1-yl)phenyl)-2,3-difluorobenzamide; (#279), N-(4-Chloro-2-(4-(3,3,3-trifluoropropyl)piperazin-1-yl)phenyl)-4-((2-(dimethylamino)acetamido)methyl)-2,3-difluorobenzamide; (#280), N-(4-(4-Chloro-2-(4-(3,3,3-trifluoropropyl)piperazin-1-yl)phenylcarbamoyl)-2,3-difluorobenzyl)-3-oxo-2,3-dihydroisoxazole-5-carboxamide; (#281), N-(4-(4-Chloro-2-(4-(3,3,3-trifluoropropyl)piperazin-1-yl)phenylcarbamoyl)-2,3-difluorobenzyl)-6-oxo-1,6-dihydropyridazine-3-carboxamide; (#282), N-(4-(4-Chloro-2-(4-(3,3,3-trifluoropropyl)piperazin-1-yl)phenylcarbamoyl)-2,3-difluorobenzyl)-2,4-dihydroxypyrimidine-5-carboxamide; (#283), N-(4-(4-Chloro-2-(4-(3,3,3-trifluoropropyl)piperazin-1-yl)phenylcarbamoyl)-2,3-difluorobenzyl)-5-oxo-4,5-dihydro-1H-1,2,4-triazole-3-carboxamide; (#284), 4-Amino-N-(4-(4-chloro-2-(4-(3,3,3-trifluoropropyl)piperazin-1-yl)phenylcarbamoyl)-2,3-difluorobenzyl)tetrahydro-2H-thiopyran-4-carboxamide; (#285), (S)-4-((2-(3-Amino-2-oxopyrrolidin-1-yl)acetamido)methyl)-N-(4-chloro-2-(4-(3,3,3-trifluoropropyl)piperazin-1-yl)phenyl)-2,3-difluorobenzamide; (#286), N-(4-Chloro-2-(4-(3,3,3-trifluoropropyl)piperazin-1-yl)phenyl)-2,3-difluoro-4-((2-oxopyrrolidin-1-yl)methyl)benzamide; (#287), N-(4-(4-Chloro-2-(4-(3,3,3-trifluoropropyl)piperazin-1-yl)phenylcarbamoyl)-2,3-difluorobenzyl)-6-hydroxynicotinamide; (#288), N-(4-(4-Chloro-2-(4-(3,3,3-trifluoropropyl)piperazin-1-yl)phenylcarbamoyl)-2,3-difluorobenzyl)-2-hydroxynicotinamide; (#289), N-(4-(4-Chloro-2-(4-(3,3,3-trifluoropropyl)piperazin-1-yl)phenylcarbamoyl)-2,3-difluorobenzyl)-2,6-dihydroxyisonicotinamide; (#290), N-(4-(4-Chloro-2-(4-(3,3,3-trifluoropropyl)piperazin-1-yl)phenylcarbamoyl)-2,3-difluorobenzyl)-2,6-dioxo-1,2,3,6-tetrahydropyrimidine-4-carboxamide; (#291), N-(4-(4-Chloro-2-(4-(3,3,3-trifluoropropyl)piperazin-1-yl)phenylcarbamoyl)-2,3-difluorobenzyl)-5-hydroxy-1-methyl-1H-pyrazole-3-carboxamide; (#292), N-(4-Chloro-2-(4-(3,3,3-trifluoropropyl)piperazin-1-yl)phenyl)-2,3-difluoro-4-((3-(3-hydroxyisoxazol-4-yl)propanamido)methyl)benzamide; (#293), 4-(4-(4-Chloro-2-(4-(3,3,3-trifluoropropyl)piperazin-1-yl)phenylcarbamoyl)-2,3-difluorobenzylamino)-4-oxobutanoic acid; (#294), N-(4-(4-Chloro-2-(4-(3,3,3-trifluoropropyl)piperazin-1-yl)phenylcarbamoyl)-2,3-difluorobenzyl)-5-hydroxypyrazine-2-carboxamide; (#295), N-(4-(4-Chloro-2-(4-(3,3,3-trifluoropropyl)piperazin-1-yl)phenylcarbamoyl)-2,3-difluorobenzyl)-6-hydroxypicolinamide; (#296), (S)—N-(4-(4-Chloro-2-(4-(3,3,3-trifluoropropyl)piperazin-1-yl)phenylcarbamoyl)-2,3-difluorobenzyl)-4-methylmorpholine-2-carboxamide; (#297), (S)—N-(4-(4-Chloro-2-(4-(3,3,3-trifluoropropyl)piperazin-1-yl)phenylcarbamoyl)-2,3-difluorobenzyl)-4-ethylmorpholine-2-carboxamide; (#298), (S)—N-(4-(4-Chloro-2-(4-(3,3,3-trifluoropropyl)piperazin-1-yl)phenylcarbamoyl)-2,3-difluorobenzyl)-4-(2-hydroxyethyl)morpholine-2-carboxamide; (#299), (S)—N-(4-(4-Chloro-2-(4-(3,3,3-trifluoropropyl)piperazin-1-yl)phenylcarbamoyl)-2,3-difluorobenzyl)-4-(3,3-dimethylbutyl)morpholine-2-carboxamide; (#300), (S)—N-(4-(4-Chloro-2-(4-(3,3,3-trifluoropropyl)piperazin-1-yl)phenylcarbamoyl)-3-fluorophenyl)morpholine-2-carboxamide; (#301), (R)—N-(4-(4-Chloro-2-(4-(3,3,3-trifluoropropyl)piperazin-1-yl)phenylcarbamoyl)-2,3-difluorobenzyl)morpholine-3-carboxamide; (#302), (R)—N-(4-(4-Chloro-2-(4-(3,3,3-trifluoropropyl)piperazin-1-yl)phenylcarbamoyl)-2,3-difluorobenzyl)-4-(2-hydroxyethyl)morpholine-3-carboxamide; (#303), (R)—N-(4-(4-Chloro-2-(4-(3,3,3-trifluoropropyl)piperazin-1-yl)phenylcarbamoyl)-2,3-difluorobenzyl)-4-ethylmorpholine-3-carboxamide; (#304), (R)—N-(4-(4-Chloro-2-(4-(3,3,3-trifluoropropyl)piperazin-1-yl)phenylcarbamoyl)-2,3-difluorobenzyl)-4-(2-hydroxyethyl)thiomorpholine-3-carboxamide; (#305), (R)—N-(4-(4-Chloro-2-(4-(3,3,3-trifluoropropyl)piperazin-1-yl)phenylcarbamoyl)-2,3-difluorobenzyl)-4-ethylthiomorpholine-3-carboxamide; (#306), N-(4-Chloro-2-(4-(3,3,3-trifluoropropyl)piperazin-1-yl)phenyl)-2,3-difluoro-4-((3-hydroxypropanamido)methyl)benzamide; (#307), N-(4-Chloro-2-(4-(3,3,3-trifluoropropyl)piperazin-1-yl)phenyl)-2,3-difluoro-4-((4-hydroxycyclohexanecarboxamido)methyl)benzamide; (#308), (R)—N-(4-Chloro-2-(4-(3,3,3-trifluoropropyl)piperazin-1-yl)phenyl)-2,3-difluoro-4-((3-hydroxypentanamido)methyl)benzamide; (#309), N-(4-Chloro-2-(4-(3,3,3-trifluoropropyl)piperazin-1-yl)phenyl)-2,3-difluoro-4-((3-hydroxypentanamido)methyl)benzamide; (#310), N-(4-Chloro-2-(4-(3,3,3-trifluoropropyl)piperazin-1-yl)phenyl)-2,3-difluoro-4-((2-hydroxy-2-methylpropanamido)methyl)benzamide; (#311), N-(4-Chloro-2-(4-(3,3,3-trifluoropropyl)piperazin-1-yl)phenyl)-2,3-difluoro-4-((1-hydroxycyclopropanecarboxamido)methyl)benzamide; (#312), N-(4-Chloro-2-(4-(3,3,3-trifluoropropyl)piperazin-1-yl)phenyl)-2,3-difluoro-4-(((1r,4r)-4-hydroxycyclohexanecarboxamido)methyl)benzamide; (#313), (R)—N-(4-Chloro-2-(4-(3,3,3-trifluoropropyl)piperazin-1-yl)phenyl)-2,3-difluoro-4-((3-hydroxybutanamido)methyl)benzamide; (#314), N-(4-Chloro-2-(4-(3,3,3-trifluoropropyl)piperazin-1-yl)phenyl)-2,3-difluoro-4-((3-hydroxy-2,2-dimethylpropanamido)methyl)benzamide; (#315), (S)—N-(4-Chloro-2-(4-(3,3,3-trifluoropropyl)piperazin-1-yl)phenyl)-2,3-difluoro-4-((3-hydroxybutanamido)methyl)benzamide; (#316), N-(4-Chloro-2-(4-(3,3,3-trifluoropropyl)piperazin-1-yl)phenyl)-2,3-difluoro-4-((4-hydroxybutanamido)methyl)benzamide; (#317), N-(4-Chloro-2-(4-(3,3,3-trifluoropropyl)piperazin-1-yl)phenyl)-4-((2-ethyl-2-hydroxybutanamido)methyl)-2,3-difluorobenzamide; (#318), N-(4-Chloro-2-(4-(3,3,3-trifluoropropyl)piperazin-1-yl)phenyl)-2,3-difluoro-4-((2-hydroxycyclohexanecarboxamido)methyl)benzamide; (#319), (R)—N-(4-Chloro-2-(4-(3,3,3-trifluoropropyl)piperazin-1-yl)phenyl)-4-((2-cyclohexyl-2-hydroxyacetamido)methyl)-2,3-difluorobenzamide; (#320), N-(4-Chloro-2-(4-(3,3,3-trifluoropropyl)piperazin-1-yl)phenyl)-2,3-difluoro-4-((3-hydroxy-3-methylbutanamido)methyl)benzamide; (#321), (S)—N-(4-Chloro-2-(4-(3,3,3-trifluoropropyl)piperazin-1-yl)phenyl)-2,3-difluoro-4-((2-hydroxy-4-methylpentanamido)methyl)benzamide; (#322), (R)—N-(4-Chloro-2-(4-(3,3,3-trifluoropropyl)piperazin-1-yl)phenyl)-2,3-difluoro-4-((2-hydroxy-4-methylpentanamido)methyl)benzamide; (#323), (2S,4R)-tert-Butyl 2-(4-(4-chloro-2-(4-(3,3,3-trifluoropropyl)piperazin-1-yl)phenylcarbamoyl)-2,3-difluorobenzylcarbamoyl)-4-hydroxypyrrolidine-1-carboxylate; (#324), tert-Butyl 3-(4-(4-chloro-2-(4-(3,3,3-trifluoropropyl)piperazin-1-yl)phenylcarbamoyl)-2,3-difluorobenzylcarbamoyl)thiomorpholine-4-carboxylate; (#325), (R)-tert-Butyl 2-(2-(4-(4-chloro-2-(4-(3,3,3-trifluoropropyl)piperazin-1-yl)phenylcarbamoyl)-2,3-difluorobenzylamino)-2-oxoethyl)piperidine-1-carboxylate; (#326), N-(4-Chloro-2-(4-(3,3,3-trifluoropropyl)piperazin-1-yl)phenyl)-2,3-difluoro-4-((3-hydroxy-2-(hydroxymethyl)-2-methylpropanamido)methyl)benzamide; (#327), N-(4-(4-Chloro-2-(4-(3,3,3-trifluoropropyl)piperazin-1-yl)phenylcarbamoyl)-2,3-difluorobenzyl)thiomorpholine-3-carboxamide; (#328), (R)—N-(4-Chloro-2-(4-(3,3,3-trifluoropropyl)piperazin-1-yl)phenyl)-2,3-difluoro-4-((2-(piperidin-2-yl)acetamido)methyl)benzamide; (#329), N-(4-Chloro-2-(4-(3,3,3-trifluoropropyl)piperazin-1-yl)phenyl)-2,3-difluoro-4-(((1r,4r)-4-(hydroxymethyl)cyclohexanecarboxamido)methyl)benzamide; (#330), N-(4-Chloro-2-(4-(3,3,3-trifluoropropyl)piperazin-1-yl)phenyl)-2,3-difluoro-4-(((1s,4s)-4-hydroxycyclohexanecarboxamido)methyl)benzamide; (#331), N-(4-Chloro-2-(4-(3,3,3-trifluoropropyl)piperazin-1-yl)phenyl)-4-((3-(dimethylamino)propanamido)methyl)-2,3-difluorobenzamide; (#332), (S)—N-(4-Chloro-2-(4-(3,3,3-trifluoropropyl)piperazin-1-yl)phenyl)-2,3-difluoro-4-((2-(pyrrolidin-3-yl)acetamido)methyl)benzamide; (#333), N-(4-Chloro-2-(4-(3,3,3-trifluoropropyl)piperazin-1-yl)phenyl)-2,3-difluoro-4-((3-(piperidin-1-yl)propanamido)methyl)benzamide; (#334), 4-(((1r,4r)-4-Aminocyclohexanecarboxamido)methyl)-N-(4-chloro-2-(4-(3,3,3-trifluoropropyl)piperazin-1-yl)phenyl)-2,3-difluorobenzamide; (#335), (S)—N-(4-Chloro-2-(4-(3,3,3-trifluoropropyl)piperazin-1-yl)phenyl)-2,3-difluoro-4-((2-(piperidin-2-yl)acetamido)methyl)benzamide; (#336), (S)—N-(4-(4-Chloro-2-(4-(3,3,3-trifluoropropyl)piperazin-1-yl)phenylcarbamoyl)-2,3-difluorobenzyl)pyrrolidine-3-carboxamide; (#337), N-(4-Chloro-2-(4-(3,3,3-trifluoropropyl)piperazin-1-yl)phenyl)-4-((3-(diethylamino)propanamido)methyl)-2,3-difluorobenzamide; (#338), 4-((2-((1s,4s)-4-Aminocyclohexyl)acetamido)methyl)-N-(4-chloro-2-(4-(3,3,3-trifluoropropyl)piperazin-1-yl)phenyl)-2,3-difluorobenzamide; (#339), N-(4-Chloro-2-(4-(3,3,3-trifluoropropyl)piperazin-1-yl)phenyl)-2,3-difluoro-4-((3-morpholinopropanamido)methyl)benzamide; (#340), N-(4-(4-Chloro-2-(4-(3,3,3-trifluoropropyl)piperazin-1-yl)phenylcarbamoyl)-2,3-difluorobenzyl)-1-methylpiperidine-4-carboxamide; (#341), 4-(((1s,4s)-4-Aminocyclohexanecarboxamido)methyl)-N-(4-chloro-2-(4-(3,3,3-trifluoropropyl)piperazin-1-yl)phenyl)-2,3-difluorobenzamide; (#342), 4-(((1R,3S)-3-Aminocyclohexanecarboxamido)methyl)-N-(4-chloro-2-(4-(3,3,3-trifluoropropyl)piperazin-1-yl)phenyl)-2,3-difluorobenzamide; (#343), (S)-4-Amino-5-(4-(4-chloro-2-(4-(3,3,3-trifluoropropyl)piperazin-1-yl)phenylcarbamoyl)-2,3-difluorobenzylamino)-5-oxopentanoic acid; (#344), (S)-2-Amino-5-(4-(4-chloro-2-(4-(3,3,3-trifluoropropyl)piperazin-1-yl)phenylcarbamoyl)-2,3-difluorobenzylamino)-5-oxopentanoic acid; (#345), 4-(((1R,3S)-3-Aminocyclopentanecarboxamido)methyl)-N-(4-chloro-2-(4-(3,3,3-trifluoropropyl)piperazin-1-yl)phenyl)-2,3-difluorobenzamide; (#346), N-(4-(4-Chloro-2-(4-(3,3,3-trifluoropropyl)piperazin-1-yl)phenylcarbamoyl)-2,3-difluorobenzyl)-1-methylpiperidine-3-carboxamide; (#347), (R)—N-(4-Chloro-2-(4-(3,3,3-trifluoropropyl)piperazin-1-yl)phenyl)-2,3-difluoro-4-((2-(piperidin-3-yl)acetamido)methyl)benzamide; (#348), (R)—N-(4-Chloro-2-(4-(3,3,3-trifluoropropyl)piperazin-1-yl)phenyl)-2,3-difluoro-4-((2-(pyrrolidin-3-yl)acetamido)methyl)benzamide; (#349), N-(4-(4-Chloro-2-(4-(3,3,3-trifluoropropyl)piperazin-1-yl)phenylcarbamoyl)-2,3-difluorobenzyl)azetidine-3-carboxamide; (#350), N-(4-Chloro-2-(4-(3,3,3-trifluoropropyl)piperazin-1-yl)phenyl)-2,3-difluoro-4-((2-(4-(hydroxymethyl)piperidin-1-yl)acetamido)methyl)benzamide; (#351), (S)—N-(4-Chloro-2-(4-(3,3,3-trifluoropropyl)piperazin-1-yl)phenyl)-2,3-difluoro-4-((2-(3-hydroxypiperidin-1-yl)acetamido)methyl)benzamide; (#352), N-(4-Chloro-2-(4-(3,3,3-trifluoropropyl)piperazin-1-yl)phenyl)-2,3-difluoro-4-((2-(piperazin-1-yl)acetamido)methyl)benzamide; (#353), (R)-4-((2-(3-Aminopyrrolidin-1-yl)acetamido)methyl)-N-(4-chloro-2-(4-(3,3,3-trifluoropropyl)piperazin-1-yl)phenyl)-2,3-difluorobenzamide; (#354), N-(4-Chloro-2-(4-(3,3,3-trifluoropropyl)piperazin-1-yl)phenyl)-2,3-difluoro-4-(((1s,4s)-4-(hydroxymethyl)cyclohexanecarboxamido)methyl)benzamide; (#355), (R)—N-(4-Chloro-2-(4-(3,3,3-trifluoropropyl)piperazin-1-yl)phenyl)-2,3-difluoro-4-((2-(2-(hydroxymethyl)morpholino)acetamido)methyl)benzamide; (#356), N-(4-Chloro-2-(4-(3,3,3-trifluoropropyl)piperazin-1-yl)phenyl)-2,3-difluoro-4-((2-(4-propylpiperazin-1-yl)acetamido)methyl)benzamide; (#357), N-(4-Chloro-2-(4-(3,3,3-trifluoropropyl)piperazin-1-yl)phenyl)-2,3-difluoro-4-((2-(5-oxo-1,4-diazepan-1-yl)acetamido)methyl)benzamide; (#358), 1-(2-(4-(4-Chloro-2-(4-(3,3,3-trifluoropropyl)piperazin-1-yl)phenylcarbamoyl)-2,3-difluorobenzylamino)-2-oxoethyl)piperidine-4-carboxamide; (#359), (R)-1-(2-(4-(4-Chloro-2-(4-(3,3,3-trifluoropropyl)piperazin-1-yl)phenylcarbamoyl)-2,3-difluorobenzylamino)-2-oxoethyl)pyrrolidine-2-carboxamide; (#360), N-(4-Chloro-2-(4-(3,3,3-trifluoropropyl)piperazin-1-yl)phenyl)-4-((2-(4-(dimethylamino)piperidin-1-yl)acetamido)methyl)-2,3-difluorobenzamide; (#361), (S)—N-(4-Chloro-2-(4-(3,3,3-trifluoropropyl)piperazin-1-yl)phenyl)-4-((2-(3-(dimethylamino)pyrrolidin-1-yl)acetamido)methyl)-2,3-difluorobenzamide; (#362), N-(4-Chloro-2-(4-(3,3,3-trifluoropropyl)piperazin-1-yl)phenyl)-2,3-difluoro-4-((2-(4-hydroxypiperidin-1-yl)acetamido)methyl)benzamide; (#363), 4-((2-(2,5-Diazabicyclo[2.2.1]heptan-2-yl)acetamido)methyl)-N-(4-chloro-2-(4-(3,3,3-trifluoropropyl)piperazin-1-yl)phenyl)-2,3-difluorobenzamide; (#364), (R)—N-(4-Chloro-2-(4-(3,3,3-trifluoropropyl)piperazin-1-yl)phenyl)-4-((2-(3-(dimethylamino)pyrrolidin-1-yl)acetamido)methyl)-2,3-difluorobenzamide; (#365), (S)—N-(4-Chloro-2-(4-(3,3,3-trifluoropropyl)piperazin-1-yl)phenyl)-2,3-difluoro-4-((2-(hexahydropyrrolo[1,2-a]pyrazin-2(1H)-yl)acetamido)methyl)benzamide; (#366), N-(4-Chloro-2-(4-(3,3,3-trifluoropropyl)piperazin-1-yl)phenyl)-2,3-difluoro-4-((2-(3-(hydroxymethyl)piperidin-1-yl)acetamido)methyl)benzamide; (#367), (S)—N-(4-Chloro-2-(4-(3,3,3-trifluoropropyl)piperazin-1-yl)phenyl)-2,3-difluoro-4-((2-(3-methylpiperazin-1-yl)acetamido)methyl)benzamide; (#368), N-(4-Chloro-2-(4-(3,3,3-trifluoropropyl)piperazin-1-yl)phenyl)-2,3-difluoro-4-((2-(4-methylpiperidin-1-yl)acetamido)methyl)benzamide; (#369), N-(4-Chloro-2-(4-(3,3,3-trifluoropropyl)piperazin-1-yl)phenyl)-2,3-difluoro-4-((2-(3-oxopiperazin-1-yl)acetamido)methyl)benzamide; (#370), 4-(2-(4-(4-Chloro-2-(4-(3,3,3-trifluoropropyl)piperazin-1-yl)phenylcarbamoyl)-2,3-difluorobenzylamino)-2-oxoethyl)piperazine-1-carboxamide; (#371), N-(4-Chloro-2-(4-(3,3,3-trifluoropropyl)piperazin-1-yl)phenyl)-2,3-difluoro-4-((2-(3-methylpiperidin-1-yl)acetamido)methyl)benzamide; (#372), N-(4-Chloro-2-(4-(3,3,3-trifluoropropyl)piperazin-1-yl)phenyl)-2,3-difluoro-4-((2-(3-hydroxypiperidin-1-yl)acetamido)methyl)benzamide; (#373), N-(4-Chloro-2-(4-(3,3,3-trifluoropropyl)piperazin-1-yl)phenyl)-2,3-difluoro-4-((2-(4-methylpiperazin-1-yl)acetamido)methyl)benzamide; (#374), N-(4-Chloro-2-(4-(3,3,3-trifluoropropyl)piperazin-1-yl)phenyl)-4-((2-(4-ethylpiperazin-1-yl)acetamido)methyl)-2,3-difluorobenzamide; (#375), N-(4-Chloro-2-(4-(3,3,3-trifluoropropyl)piperazin-1-yl)phenyl)-2,3-difluoro-4-((2-(2-(2-hydroxyethyl)piperidin-1-yl)acetamido)methyl)benzamide; (#376), (S)—N-(4-Chloro-2-(4-(3,3,3-trifluoropropyl)piperazin-1-yl)phenyl)-2,3-difluoro-4-((2-(3-hydroxypyrrolidin-1-yl)acetamido)methyl)benzamide; (#377), (R)—N-(4-Chloro-2-(4-(3,3,3-trifluoropropyl)piperazin-1-yl)phenyl)-2,3-difluoro-4-((2-(2-(hydroxymethyl)pyrrolidin-1-yl)acetamido)methyl)benzamide; (#378), (S)—N-(4-Chloro-2-(4-(3,3,3-trifluoropropyl)piperazin-1-yl)phenyl)-2,3-difluoro-4-((2-(2-(hydroxymethyl)pyrrolidin-1-yl)acetamido)methyl)benzamide; (#379), 1-(2-(4-(4-Chloro-2-(4-(3,3,3-trifluoropropyl)piperazin-1-yl)phenylcarbamoyl)-2,3-difluorobenzylamino)-2-oxoethyl)piperidine-3-carboxamide; (#380), (S)-1-(2-(4-(4-Chloro-2-(4-(3,3,3-trifluoropropyl)piperazin-1-yl)phenylcarbamoyl)-2,3-difluorobenzylamino)-2-oxoethyl)pyrrolidine-2-carboxamide; (#381), (1r,4r)-4-(4-(4-Chloro-2-(4-(3,3,3-trifluoropropyl)piperazin-1-yl)phenylcarbamoyl)-2,3-difluorobenzylcarbamoyl)cyclohexyl dihydrogen phosphate; (#382), 2-(4-(4-Chloro-2-(4-(3,3,3-trifluoropropyl)piperazin-1-yl)phenylcarbamoyl)-2,3-difluorobenzylamino)-2-oxoethyl dihydrogen phosphate; (#383), (R)-tert-Butyl 1-(4-(4-chloro-2-(4-(3,3,3-trifluoropropyl)piperazin-1-yl)phenylcarbamoyl)-3-fluorobenzylcarbamoyl)pyrrolidin-3-ylcarbamate; (#384), (S)-tert-Butyl 1-(4-(4-chloro-2-(4-(3,3,3-trifluoropropyl)piperazin-1-yl)phenylcarbamoyl)-3-fluorobenzylcarbamoyl)pyrrolidin-3-ylcarbamate; (#385), N-(4-Chloro-2-(4-(3,3,3-trifluoropropyl)piperazin-1-yl)phenyl)-2,3-difluoro-4-((4-(hydroxymethyl)cyclohexanecarboxamido)methyl)benzamide; (#386), (S)-4-(2-Amino-4-methylpentanamido)methyl)-N-(4-chloro-2-(4-(3,3,3-trifluoropropyl)piperazin-1-yl)phenyl)-2,3-difluorobenzamide; (#387), (S)-4-(2-Amino-3-cyanopropanamido)methyl)-N-(4-chloro-2-(4-(3,3,3-trifluoropropyl)piperazin-1-yl)phenyl)-2,3-difluorobenzamide; (#388), 4-Amino-N-(4-(4-chloro-2-(4-(3,3,3-trifluoropropyl)piperazin-1-yl)phenylcarbamoyl)-2,3-difluorobenzyl)-1,1-dioxotetrahydro-2H-thiopyran-4-carboxamide; (#389), (S)-2-Amino-N1-(4-(4-chloro-2-(4-(3,3,3-trifluoropropyl)piperazin-1-yl)phenylcarbamoyl)-2,3-difluorobenzyl)succinamide; (#390), 4-((3-Amino-2-hydroxypropanamido)methyl)-N-(4-chloro-2-(4-(3,3,3-trifluoropropyl)piperazin-1-yl)phenyl)-2,3-difluorobenzamide; (#391), N-(4-Chloro-2-(4-(3,3,3-trifluoropropyl)piperazin-1-yl)phenyl)-2,3-difluoro-4-((2-hydroxypropanamido)methyl)benzamide; (#392), (1s,4s)-4-(4-(4-Chloro-2-(4-(3,3,3-trifluoropropyl)piperazin-1-yl)phenylcarbamoyl)-2,3-difluorobenzylcarbamoyl)cyclohexyl dihydrogen phosphate; (#393), N-(4-(4-Chloro-2-(4-(3,3,3-trifluoropropyl)piperazin-1-yl)phenylcarbamoyl)-2,3-difluorobenzyl)-5-(hydroxymethyl)-1H-1,2,3-triazole-4-carboxamide; (#394), N-(4-(4-Chloro-2-(4-(3,3,3-trifluoropropyl)piperazin-1-yl)phenylcarbamoyl)-3-fluorobenzyl)piperazine-1-carboxamide; (#395), N-(4-(4-Chloro-2-(4-(3,3,3-trifluoropropyl)piperazin-1-yl)phenylcarbamoyl)-3-fluorobenzyl)-4-ethylpiperazine-1-carboxamide; (#396), N-(4-(4-Chloro-2-(4-(3,3,3-trifluoropropyl)piperazin-1-yl)phenylcarbamoyl)-3-fluorobenzyl)-1,1-dioxo-thiomorpholine-4-carboxamide; (#397), (S)—N-(4-(4-Chloro-2-(4-(3,3,3-trifluoropropyl)piperazin-1-yl)phenylcarbamoyl)-3-fluorobenzyl)-3-hydroxypyrrolidine-1-carboxamide; (#398), N-(4-(4-Chloro-2-(4-(3,3,3-trifluoropropyl)piperazin-1-yl)phenylcarbamoyl)-3-fluorobenzyl)-4-(2-hydroxyethyl)piperazine-1-carboxamide; (#399), N-(4-(4-Chloro-2-(4-(3,3,3-trifluoropropyl)piperazin-1-yl)phenylcarbamoyl)-3-fluorobenzyl)-4-(hydroxymethyl)piperidine-1-carboxamide; (#400), (S)-3-Amino-N-(4-(4-chloro-2-(4-(3,3,3-trifluoropropyl)piperazin-1-yl)phenylcarbamoyl)-3-fluorobenzyl)piperidine-1-carboxamide; (#401), N-(4-(4-Chloro-2-(4-(3,3,3-trifluoropropyl)piperazin-1-yl)phenylcarbamoyl)-3-fluorobenzyl)-3-hydroxyazetidine-1-carboxamide; (#402), (R)-3-Amino-N-(4-(4-chloro-2-(4-(3,3,3-trifluoropropyl)piperazin-1-yl)phenylcarbamoyl)-3-fluorobenzyl)pyrrolidine-1-carboxamide; (#403), (S)-3-Amino-N-(4-(4-chloro-2-(4-(3,3,3-trifluoropropyl)piperazin-1-yl)phenylcarbamoyl)-3-fluorobenzyl)pyrrolidine-1-carboxamide; (#404), N-(4-(4-Chloro-2-(4-(3,3,3-trifluoropropyl)piperazin-1-yl)phenylcarbamoyl)-2,3-difluorobenzyl)piperazine-1-carboxamide; (#405), N-(4-(4-Chloro-2-(4-(3,3,3-trifluoropropyl)piperazin-1-yl)phenylcarbamoyl)-2,3-difluorobenzyl)-4-(2-hydroxyethyl)piperazine-1-carboxamide; (#406), (S)—N-(4-(4-Chloro-2-(4-(3,3,3-trifluoropropyl)piperazin-1-yl)phenylcarbamoyl)-2,3-difluorobenzyl)-3-hydroxypyrrolidine-1-carboxamide; (#407), N-(4-(4-Chloro-2-(4-(3,3,3-trifluoropropyl)piperazin-1-yl)phenylcarbamoyl)-2,3-difluorobenzyl)-4-(hydroxymethyl)piperidine-1-carboxamide; (#408), (S)—N¹-(4-(4-Chloro-2-(4-(3,3,3-trifluoropropyl)piperazin-1-yl)phenylcarbamoyl)-2,3-difluorobenzyl)pyrrolidine-1,2-dicarboxamide; (#409), N-(4-(4-Chloro-2-(4-(3,3,3-trifluoropropyl)piperazin-1-yl)phenylcarbamoyl)-2,3-difluorobenzyl)-4-(dimethylamino)piperidine-1-carboxamide; (#410), N-(4-(4-Chloro-2-(4-(3,3,3-trifluoropropyl)piperazin-1-yl)phenylcarbamoyl)-2,3-difluorobenzyl)-1,1-dioxo-thiomorpholine-4-carboxamide; (#411), N¹-(4-(4-Chloro-2-(4-(3,3,3-trifluoropropyl)piperazin-1-yl)phenylcarbamoyl)-2,3-difluorobenzyl)piperazine-1,4-dicarboxamide; (#412), N-(4-(4-Chloro-2-(4-(3,3,3-trifluoropropyl)piperazin-1-yl)phenylcarbamoyl)-2,3-difluorobenzyl)-3-oxopiperazine-1-carboxamide; (#413), (R)—N-(4-(4-Chloro-2-(4-(3,3,3-trifluoropropyl)piperazin-1-yl)phenylcarbamoyl)-2,3-difluorobenzyl)-2-(hydroxymethyl)pyrrolidine-1-carboxamide; (#414), N-(4-(4-Chloro-2-(4-(3,3,3-trifluoropropyl)piperazin-1-yl)phenylcarbamoyl)-2,3-difluorobenzyl)-2-(2-hydroxyethyl)piperidine-1-carboxamide; (#415), N-(4-(4-Chloro-2-(4-(3,3,3-trifluoropropyl)piperazin-1-yl)phenylcarbamoyl)-2,3-difluorobenzyl)-3-hydroxyazetidine-1-carboxamide; (#416), N-(4-(4-Chloro-2-(4-(3,3,3-trifluoropropyl)piperazin-1-yl)phenylcarbamoyl)-2,3-difluorobenzyl)-2-(hydroxymethyl)morpholine-4-carboxamide; (#417), N-(4-(4-Chloro-2-(4-(3,3,3-trifluoropropyl)piperazin-1-yl)phenylcarbamoyl)-3-fluorophenyl)-4-(hydroxymethyl)piperidine-1-carboxamide; (#418), 1-(4-(4-Chloro-2-(4-(3,3,3-trifluoropropyl)piperazin-1-yl)phenylcarbamoyl)-2,3-difluorobenzylcarbamoyl)azetidine-3-carboxylic acid; (#419), (R)—N-(4-(4-Chloro-2-(4-(3,3,3-trifluoropropyl)piperazin-1-yl)phenylcarbamoyl)-2,3-difluorobenzyl)-2-(hydroxymethyl)morpholine-4-carboxamide; (#420), N-(4-(4-Chloro-2-(4-(3,3,3-trifluoropropyl)piperazin-1-yl)phenylcarbamoyl)-2,3-difluorobenzyl)-4-(3-hydroxypropyl)piperidine-1-carboxamide; (#421), N-(4-(4-Chloro-2-(4-(3,3,3-trifluoropropyl)piperazin-1-yl)phenylcarbamoyl)-2,3-difluorobenzyl)-3-hydroxypiperidine-1-carboxamide; (#422), N-(4-(4-Chloro-2-(4-(3,3,3-trifluoropropyl)piperazin-1-yl)phenylcarbamoyl)-2,3-difluorobenzyl)-4-cyanopiperidine-1-carboxamide; (#423), N-(4-(4-Chloro-2-(4-(3,3,3-trifluoropropyl)piperazin-1-yl)phenylcarbamoyl)-2,3-difluorobenzyl)-2-(hydroxymethyl)piperidine-1-carboxamide; (#424), N-(4-(4-Chloro-2-(4-(3,3,3-trifluoropropyl)piperazin-1-yl)phenylcarbamoyl)-2,3-difluorobenzyl)-4-hydroxypiperidine-1-carboxamide; (#425), (R)—N-(4-(4-Chloro-2-(4-(3,3,3-trifluoropropyl)piperazin-1-yl)phenylcarbamoyl)-2,3-difluorobenzyl)-3-hydroxypyrrolidine-1-carboxamide; (#426), N-(4-(4,5-Dichloro-2-(4-(3,3,3-trifluoropropyl)piperazin-1-yl)phenylcarbamoyl)-2,3-difluorobenzyl)-4-(hydroxymethyl)piperidine-1-carboxamide; (#427), N-(4-(4-Chloro-5-fluoro-2-(4-(3,3,3-trifluoropropyl)piperazin-1-yl)phenylcarbamoyl)-2,3-difluorobenzyl)-4-(hydroxymethyl)piperidine-1-carboxamide; (#428), N-(4-Chloro-2-(4-(3,3,3-trifluoropropyl)piperazin-1-yl)phenyl)-4-((2,5-dioxoimidazolidin-1-yl)methyl)-2,3-difluorobenzamide; (#429), N-(4-Chloro-2-(4-(3,3,3-trifluoropropyl)piperazin-1-yl)phenyl)-4-((2,6-dioxotetrahydropyrimidin-1(2H)-yl)methyl)-2,3-difluorobenzamide; (#430), N-(4-(4-Chloro-2-(4-(3,3,3-trifluoropropyl)piperazin-1-yl)phenylcarbamoyl)-2,3-difluorobenzyl)-2-oxopyrrolidine-1-carboxamide; (#431), N-(4-Chloro-2-(4-(3,3,3-trifluoropropyl)piperazin-1-yl)phenyl)-2,3-difluoro-4-((3-methyl-2,5-dioxoimidazolidin-1-yl)methyl)benzamide; (#432), (S)—N-(4-Chloro-2-(4-(3,3,3-trifluoropropyl)piperazin-1-yl)phenyl)-2,3-difluoro-4-((4-isobutyl-2,5-dioxoimidazolidin-1-yl)methyl)benzamide; (#433), (S)—N-(4-(4-Chloro-2-(4-(3,3,3-trifluoropropyl)piperazin-1-yl)phenylcarbamoyl)-2,3-difluorobenzyl)-3-(hydroxymethyl)piperidine-1-carboxamide; (#434), N-(4-(4,5-Difluoro-2-(4-(3,3,3-trifluoropropyl)piperazin-1-yl)phenylcarbamoyl)-2,3-difluorobenzyl)-4-(hydroxymethyl)piperidine-1-carboxamide; (#435), (R)—N-(4-(4-Chloro-2-(4-(3,3,3-trifluoropropyl)piperazin-1-yl)phenylcarbamoyl)-2,3-difluorobenzyl)-3-(hydroxymethyl)piperidine-1-carboxamide; (#436), (R)—N-(4-(4-Chloro-2-(4-(3,3,3-trifluoropropyl)piperazin-1-yl)phenylcarbamoyl)-2,3-difluorobenzyl)-3-hydroxypiperidine-1-carboxamide; (#437), (S)—N-(4-(4-Chloro-2-(4-(3,3,3-trifluoropropyl)piperazin-1-yl)phenylcarbamoyl)-2,3-difluorobenzyl)-3-hydroxypiperidine-1-carboxamide; (#438), (S)—N-(4-(4-Chloro-2-(4-(3,3,3-trifluoropropyl)piperazin-1-yl)phenylcarbamoyl)-2,3-difluorobenzyl)-2-(hydroxymethyl)pyrrolidine-1-carboxamide; (#439), (R)—N-(4-(4-Chloro-2-(4-(3,3,3-trifluoropropyl)piperazin-1-yl)phenylcarbamoyl)-2,3-difluorobenzyl)-3-(hydroxymethyl)pyrrolidine-1-carboxamide; (#440), (S)-1-(4-(4-Chloro-2-(4-(3,3,3-trifluoropropyl)piperazin-1-yl)phenylcarbamoyl)-2,3-difluorobenzylcarbamoyl)pyrrolidin-3-yl dihydrogen phosphate; (#441), (R)-1-(4-(4-Chloro-2-(4-(3,3,3-trifluoropropyl)piperazin-1-yl)phenylcarbamoyl)-2,3-difluorobenzylcarbamoyl)pyrrolidin-3-yl dihydrogen phosphate; (#442), (S)-tert-Butyl 1-(4-(4-chloro-2-(4-(3,3,3-trifluoropropyl)piperazin-1-yl)phenylcarbamoyl)-3-fluorobenzylcarbamoyl)piperidin-3-ylcarbamate; (#443), tert-Butyl 4-(4-(4-chloro-2-(4-(3,3,3-trifluoropropyl)piperazin-1-yl)phenylcarbamoyl)-2,3-difluorobenzylcarbamoyl)piperidine-1-carboxylate; (#444), tert-Butyl (1S,2R)-2-(4-(4-chloro-2-(4-(3,3,3-trifluoropropyl)piperazin-1-yl)phenylcarbamoyl)-2,3-difluorobenzylcarbamoyl)cyclohexylcarbamate; (#445), (S)-tert-Butyl 3-(4-(4-chloro-2-(4-(3,3,3-trifluoropropyl)piperazin-1-yl)phenylcarbamoyl)-2,3-difluorobenzylcarbamoyl)piperidine-1-carboxylate; (#446), tert-Butyl 2-(4-(4-chloro-2-(4-(3,3,3-trifluoropropyl)piperazin-1-yl)phenylcarbamoyl)-2,3-difluorobenzylcarbamoyl)morpholine-4-carboxylate; (#447), tert-Butyl 3-(4-(4-chloro-2-(4-(3,3,3-trifluoropropyl)piperazin-1-yl)phenylcarbamoyl)-2,3-difluorobenzylamino)-3-oxopropylcarbamate; (#448), tert-Butyl 2-(4-(4-chloro-2-(4-(3,3,3-trifluoropropyl)piperazin-1-yl)phenylcarbamoyl)-2,3-difluorobenzylamino)-2-oxoethylcarbamate; (#449), tert-Butyl 3-(4-(4-chloro-2-(4-(3,3,3-trifluoropropyl)piperazin-1-yl)phenylcarbamoyl)-2,3-difluorobenzylamino)-2-hydroxy-3-oxopropylcarbamate; (#450), (S)-tert-Butyl 1-(4-(4-chloro-2-(4-(3,3,3-trifluoropropyl)piperazin-1-yl)phenylcarbamoyl)-2,3-difluorobenzylamino)-1-oxopropan-2-ylcarbamate; (#451), (S)-tert-Butyl 1-(4-(4-chloro-2-(4-(3,3,3-trifluoropropyl)piperazin-1-yl)phenylcarbamoyl)-2,3-difluorobenzylamino)-3-hydroxy-1-oxopropan-2-ylcarbamate; (#452), (S)-tert-Butyl 2-(4-(4-chloro-2-(4-(3,3,3-trifluoropropyl)piperazin-1-yl)phenylcarbamoyl)-2,3-difluorobenzylcarbamoyl)morpholine-4-carboxylate; (#453), (R)-tert-Butyl 2-(4-(4-chloro-2-(4-(3,3,3-trifluoropropyl)piperazin-1-yl)phenylcarbamoyl)-2,3-difluorobenzylcarbamoyl)morpholine-4-carboxylate; (#454), (R)-tert-Butyl 3-(4-(4-chloro-2-(4-(3,3,3-trifluoropropyl)piperazin-1-yl)phenylcarbamoyl)-2,3-difluorobenzylcarbamoyl)thiomorpholine-4-carboxylate; (#455), (S)-tert-Butyl 2-(4-(4-chloro-2-(4-(3,3,3-trifluoropropyl)piperazin-1-yl)phenylcarbamoyl)-2,3-difluorobenzylcarbamoyl)pyrrolidine-1-carboxylate; (#456), (R)-tert-Butyl 1-(4-(4-chloro-2-(4-(3,3,3-trifluoropropyl)piperazin-1-yl)phenylcarbamoyl)-2,3-difluorobenzylamino)-1-oxopropan-2-ylcarbamate; (#457), (2S)-tert-Butyl 2-(4-(4-chloro-2-(4-(3,3,3-trifluoropropyl)piperazin-1-yl)phenylcarbamoyl)-2,3-difluorobenzylcarbamoyl)-4-hydroxypyrrolidine-1-carboxylate; (#458), tert-Butyl 4-(4-(4-chloro-2-(4-(3,3,3-trifluoropropyl)piperazin-1-yl)phenylcarbamoyl)-2,3-difluorobenzylcarbamoyl)tetrahydro-2H-thiopyran-4-ylcarbamate; (#459), tert-Butyl 4-(4-(4-chloro-2-(4-(3,3,3-trifluoropropyl)piperazin-1-yl)phenylcarbamoyl)-2,3-difluorobenzylamino)-4-oxobutanoate; (#460), (R)-tert-Butyl 2-(4-(4-chloro-2-(4-(3,3,3-trifluoropropyl)piperazin-1-yl)phenylcarbamoyl)-2,3-difluorophenylcarbamoyl)morpholine-4-carboxylate; (#461), (S)-tert-Butyl 1-(2-(4-(4-chloro-2-(4-(3,3,3-trifluoropropyl)piperazin-1-yl)phenylcarbamoyl)-2,3-difluorobenzylamino)-2-oxoethyl)-2-oxopyrrolidin-3-ylcarbamate; (#462), (S)-tert-Butyl 2-(4-(4-chloro-2-(4-(3,3,3-trifluoropropyl)piperazin-1-yl)phenylcarbamoyl)-3-fluorophenylcarbamoyl)morpholine-4-carboxylate; (#463), (S)-tert-Butyl 4-amino-1-(4-(4-chloro-2-(4-(3,3,3-trifluoropropyl)piperazin-1-yl)phenylcarbamoyl)-2,3-difluorobenzylamino)-1,4-dioxobutan-2-ylcarbamate; (#464), (S)-tert-Butyl 3-(2-(4-(4-chloro-2-(4-(3,3,3-trifluoropropyl)piperazin-1-yl)phenylcarbamoyl)-2,3-difluorobenzylamino)-2-oxoethyl)pyrrolidine-1-carboxylate; (#465), tert-Butyl (1r,4r)-4-(4-(4-chloro-2-(4-(3,3,3-trifluoropropyl)piperazin-1-yl)phenylcarbamoyl)-2,3-difluorobenzylcarbamoyl)cyclohexylcarbamate; (#466), (S)-tert-Butyl 2-(2-(4-(4-chloro-2-(4-(3,3,3-trifluoropropyl)piperazin-1-yl)phenylcarbamoyl)-2,3-difluorobenzylamino)-2-oxoethyl)piperidine-1-carboxylate; (#467), (S)-tert-Butyl 3-(4-(4-chloro-2-(4-(3,3,3-trifluoropropyl)piperazin-1-yl)phenylcarbamoyl)-2,3-difluorobenzylcarbamoyl)pyrrolidine-1-carboxylate; (#468), tert-Butyl (1s,4s)-4-(2-(4-(4-chloro-2-(4-(3,3,3-trifluoropropyl)piperazin-1-yl)phenylcarbamoyl)-2,3-difluorobenzylamino)-2-oxoethyl)cyclohexylcarbamate; (#469), tert-Butyl (1s,4s)-4-(4-(4-chloro-2-(4-(3,3,3-trifluoropropyl)piperazin-1-yl)phenylcarbamoyl)-2,3-difluorobenzylcarbamoyl)cyclohexylcarbamate; (#470), tert-Butyl (1S,3R)-3-(4-(4-chloro-2-(4-(3,3,3-trifluoropropyl)piperazin-1-yl)phenylcarbamoyl)-2,3-difluorobenzylcarbamoyl)cyclohexylcarbamate; (#471), (S)-4-(tert-Butoxycarbonylamino)-5-(4-(4-chloro-2-(4-(3,3,3-trifluoropropyl)piperazin-1-yl)phenylcarbamoyl)-2,3-difluorobenzylamino)-5-oxopentanoic acid; (#472), (S)-2-(tert-Butoxycarbonylamino)-5-(4-(4-chloro-2-(4-(3,3,3-trifluoropropyl)piperazin-1-yl)phenylcarbamoyl)-2,3-difluorobenzylamino)-5-oxopentanoic acid; (#473), tert-Butyl (1S,3R)-3-(4-(4-chloro-2-(4-(3,3,3-trifluoropropyl)piperazin-1-yl)phenylcarbamoyl)-2,3-difluorobenzylcarbamoyl)cyclopentylcarbamate; (#474), (R)-tert-Butyl 3-(2-(4-(4-chloro-2-(4-(3,3,3-trifluoropropyl)piperazin-1-yl)phenylcarbamoyl)-2,3-difluorobenzylamino)-2-oxoethyl)piperidine-1-carboxylate; (#475), (R)-tert-Butyl 3-(2-(4-(4-chloro-2-(4-(3,3,3-trifluoropropyl)piperazin-1-yl)phenylcarbamoyl)-2,3-difluorobenzylamino)-2-oxoethyl)pyrrolidine-1-carboxylate; (#476), tert-Butyl 3-(4-(4-chloro-2-(4-(3,3,3-trifluoropropyl)piperazin-1-yl)phenylcarbamoyl)-2,3-difluorobenzylcarbamoyl)azetidine-1-carboxylate; (#477), (R)-tert-Butyl 1-(2-(4-(4-chloro-2-(4-(3,3,3-trifluoropropyl)piperazin-1-yl)phenylcarbamoyl)-2,3-difluorobenzylamino)-2-oxoethyl)pyrrolidin-3-ylcarbamate; (#478), (S)-tert-Butyl 4-(2-(4-(4-chloro-2-(4-(3,3,3-trifluoropropyl)piperazin-1-yl)phenylcarbamoyl)-2,3-difluorobenzylamino)-2-oxoethyl)-2-methylpiperazine-1-carboxylate; (#479), (S)-tert-Butyl 1-(4-(4-chloro-2-(4-(3,3,3-trifluoropropyl)piperazin-1-yl)phenylcarbamoyl)-2,3-difluorobenzylamino)-4-methyl-1-oxopentan-2-ylcarbamate; (#480), (S)-tert-Butyl 1-(4-(4-chloro-2-(4-(3,3,3-trifluoropropyl)piperazin-1-yl)phenylcarbamoyl)-2,3-difluorobenzylamino)-3-cyano-1-oxopropan-2-ylcarbamate; and (#481), tert-Butyl 4-(4-(4-chloro-2-(4-(3,3,3-trifluoropropyl)piperazin-1-yl)phenylcarbamoyl)-2,3-difluorobenzylcarbamoyl)-1,1-dioxotetrahydro-2H-thiopyran-4-ylcarbamate.

Additionally, individual compounds and chemical genera of the present invention, for example those compounds found in the directly above group, including diastereoisomers and enantiomers thereof, encompass all pharmaceutically acceptable salts, solvates, and hydrates, thereof.

The compounds of Formula (I) of the present invention may be prepared according to relevant published literature procedures that are used by one skilled in the art. Exemplary reagents and procedures for these reactions appear hereinafter in the working Examples. Protection and deprotection may be carried out by procedures generally known in the art (see, for example, Greene, T. W. and Wuts, P. G. M., Protecting Groups in Organic Synthesis, 3^{rd} Edition, 1999 [Wiley]).

It is understood that the present invention embraces each diastereoisomer, each enantiomer and mixtures thereof of each compound and generic formulae disclosed herein just as if they were each individually disclosed with the specific stereochemical designation for each chiral carbon. Separation of the individual isomers (such as, by chiral HPLC, recrystallization of diastereoisomeric mixtures and the like) or selective synthesis (such as, by enantiomeric selective syntheses and the like) of the individual isomers is accomplished by application of various methods which are well known to practitioners in the art.

### Certain Embodiments

### Compositions and Methods Related Thereto

One aspect of the present invention pertains to compositions comprising a compound of the present invention. One aspect of the present invention pertains to pharmaceutical products selected from: a pharmaceutical composition, a formulation, a unit dosage form, and a kit; each comprising a compound of the present invention. One aspect of the present invention pertains to pharmaceutical compositions comprising a compound of the present invention, and a pharmaceutically acceptable carrier. One aspect of the present invention pertains to methods for preparing a pharmaceutical composition comprising the step of admixing a compound of the present invention, and a pharmaceutically acceptable carrier; some embodiments pertain to pharmaceutical compositions obtained by any of the methods described herein. One aspect of the present invention pertains to compositions comprising a compound of the present invention, and a second pharmaceutical agent.

In any of the embodiments that recites the terms “a pharmaceutical agent” and “a second pharmaceutical agent”, it is appreciated that these terms in some aspects be further limited to a pharmaceutical agent/second pharmaceutical agent that is not a compound of Formula (I) or a compounds related thereto. It is understood that the terms “a pharmaceutical agent” and “a second pharmaceutical agent” may refer to a pharmaceutical agent that is not detectable or has an IC₅₀ that is greater than a value selected from: 50 μM, 10 μM, 1 μM, and 0.1 μM in a Mas receptor activity assay as described in Example 3, such as the HTRF assay in Example 3.1.

One aspect of the present invention pertains to methods for preparing a composition comprising the step of admixing a compound of the present invention, and a second pharmaceutical agent; some embodiments pertain to compositions obtained by any of the methods described herein. One aspect of the present invention pertains to pharmaceutical products selected from: a pharmaceutical composition, a formulation, a unit dosage form, a combined preparation, a twin pack, and a kit; each comprising a compound of the present invention, and a second pharmaceutical agent. One aspect of the present invention pertains to pharmaceutical compositions comprising a compound of the present invention, a second pharmaceutical agent, and a pharmaceutically acceptable carrier. One aspect of the present invention pertains to methods for preparing a pharmaceutical composition comprising the step of admixing a compound of the present invention, a second pharmaceutical agent, and a pharmaceutically acceptable carrier; some embodiments pertain to pharmaceutical compositions obtained by any of the methods described herein.

### Certain Embodiments

### Methods, Pharmaceutical Products, Combinations, and Uses of the Present Invention

### Mas Receptor

In mammals, Mas is expressed predominantly in brain and testis with moderate levels of expression in heart and kidney, and lower expression in several other tissues (Alenina N., et al., Exp Physiol 93:528-537 (2008); Metzger R., et al., FEBS Lett 357:27-32 (1995); Villar A. J. and Pedersen R. A., Nat Genet. 8:373-379 (1994); Young D., et al., Cell 45:711-719 (1986)). As further described herein, Mas is expressed in cardiovascular tissue (Example 5.5). In the mouse heart, low levels of Mas mRNA transcripts have been detected in cardiomyocytes and higher concentrations in the endothelium of coronary arteries (Alenina N., et al., Exp Physiol 93:528-537 (2008)). We confirm Mas mRNA and protein expression in rat hearts, with cardiomyocytes and coronary arteries as sites of enriched expression (Example 5.5). Additionally, by co-localization studies we determined that both smooth muscle cells and endothelial cells in coronary arteries express Mas. Most importantly, as shown herein, the Mas is expressed in human heart (Example 5.5). Consistent with its expression in rodents, Mas is expressed in all human cardiac chambers, and in both human cardiomyocytes and human coronary arteries indicating that Mas plays a role in human heart function.

In addition, small molecule/non-peptide modulators were used to confirm the Mas-G_{q}-PLC signaling pathway (Example 3 and Example 5.7). These results demonstrate that Mas is a G_{q}-coupled receptor. The Mas receptor was discovered more than two decades ago. Based on the sequence, Mas was predicted to be a GPCR; however, an understanding of its intracellular signaling pathways has been slow to develop. Some studies have suggested that Mas may couple to G_{q} or Gᵢ (Bikkavilli R. K., et al., Biochem Pharmacol 71:319-337, (2006); Canals M, et al., J Biol Chem 281:16757-16767 (2006); and Singh A, et al., J Mol Signal 5:11 (2010)). As disclosed herein we examined the G-protein coupling of Mas in both HEK293 cells and in the more biologically relevant cardiac myocytes. Our data demonstrate that in both cell types Mas constitutively couples to the G_{q} protein, which in turn activates PLC and causes inositol phosphate accumulation (Example 3). Mas-G_{q} coupling was confirmed using a novel Mas agonist and inverse agonists. These ligands modulated Mas-dependent IP accumulation and calcium mobilization in a dose-dependent manner. The absence of constitutive adenylate cyclase activity in the same cellular context suggests that Mas couples preferentially to G_{q}, although Gᵢ coupling was activated by high concentrations of the Mas agonist. Our results demonstrate higher sensitivity for G_{q} coupling (IC₅₀=0.351±0.055 μM) with agonist AR234960 compared to Gᵢ coupling (IC₅₀=0.719±0.012 μM). In addition, the preferred G_{q} coupling observed with the Mas agonist was also observed in cells expressing rat Mas (Table C and D) and was confirmed with additional Mas-G_{q} agonists (Example 3).

Although there is currently no direct evidence that Mas signals via activation of G₁₂/G₁₃ it has been reported that Mas transformation in NIH 3T3 cells is mediated through Rac1, a member of the Rho family proteins (Zohn I. E., et al., Mol Cell Biol 18:1225-1235 (1998)). Data described herein do not rule out the possibility that Mas is also coupled to G₁₂/G₁₃ in the heart.

While we have observed G_{q} coupling of the Mas receptor in Mas-expressing cells in vitro, it was also important to confirm the signaling pathway of the endogenous Mas receptor in the heart. To address this, and the relationship between Mas signaling and biological function in the heart, the ex vivo coronary flow functional assay and the in vivo coronary artery ligation model was utilized as described herein.

Agonist stimulation of arterial smooth muscle G_{q}-coupled receptors (e.g. endothelin ET_{A}), results in increased cytosolic Ca²⁺, vasoconstriction and decreased arterial blood flow (Seo B., et al., Circulation 89:1203-1208 (1994); and Wynne B. M., et al., J Am Soc Hypertens 3:84-95 (2009)). Conversely, pharmacological blockade of the ET_{A} receptor results in vasodilation and increased coronary flow (Halcox J. P., et al., Hypertension 49:1134-1141, 2007; and Kyriakides Z. S., et al., Heart 84:176-182 (2000)). Therefore, the activation of the G_{q}-coupled Mas receptor in coronary arteries would lead to vasoconstriction and decreased coronary flow. In the present study, we have demonstrated that Mas-G_{q} agonist treatment causes vasoconstriction resulting in decreased coronary flow and conversely, Mas inverse agonist treatment results in vasodilation and increased coronary flow. Inhibition of PLC attenuated the effect of the Mas agonist, confirming that vasoconstriction via the endogenous Mas receptor is mediated through the G_{q}-PLC pathway. The observation that Mas agonist-induced vasoconstriction is preserved in endothelium-denuded hearts indicates that this response is mediated by Mas receptors on smooth muscle cells in coronary arteries.

The Mas receptor has been implicated in the regulation of cardiac function during ischemia and reperfusion in isolated hearts (Castro C. H., et al., Life Sci 80:264-268 (2006)). To determine whether the Mas receptor also plays a role in regional ischemia/reperfusion injury in vivo, we performed coronary artery ligation studies in Mas^{+/+} (wild type) and Mas^{-/-} (Mas knockout) mice. The data demonstrate that infarct size is significantly reduced in Mas^{-/-} mice after ischemia/reperfusion (Example 5.6). Interestingly, ablation of Mas expression in mice also renders the kidneys resistant to ischemia/reperfusion injury (Esteban V., et al., PLoS One 4:e5406 (2009)). Finally, we confirmed a role for Mas in myocardial ischemia/reperfusion injury by treating rats with Mas inverse agonists either prior to ischemia or immediately before reperfusion. Both treatment protocols resulted in reduced infarct size, indicating that excessive Mas-G_{q} signaling occurs both during ischemia and during reperfusion. These data are consistent with the cardioprotection observed with other inhibitors of other myocardial G_{q}-coupled receptors in the setting of reperfusion injury (Watanabe T., et al., Br J Pharmacol 114:949-954, 1995; and Dai W., et al., Cardiovasc Ther 28:30-37 (2010)). Importantly, the decreased infarct size observed with Mas inverse agonist treatment in vivo resulted in improved long-term cardiac function.

One mechanism whereby Mas inverse agonists and antagonists provide cardioprotection is by improving cardiac blood flow. It has been suggested that reduced coronary flow is an important factor that contributes to ischemia/reperfusion injury (Collard C. D. and Gelman S., Anesthesiology 94:1133-1138 (2001)). The Mas receptor present on arterial smooth muscle cells promotes vasoconstriction, whereas inhibition of Mas signaling by Mas inverse agonists promotes vasodilation, resulting in improved blood flow. Thus, it is likely that the improved coronary flow resulting from Mas inverse agonist treatment accounts, at least in part, for the cardioprotective properties of these compounds.

A second mechanism whereby Mas inverse agonists can provide cardioprotection is by reducing apoptosis. Mas is expressed in cardiomyocytes where ischemia/reperfusion is known to cause a marked increase in intracellular Ca²⁺ content (Tani M. and Neely J. R., Circ Res 65:1045-1056 (1989); and Murphy E. and Steenbergen C., Physiol Rev 88:581-609 (2008)). Cytosolic and subsequent mitochondrial Ca²⁺ overload results in cell death during myocardial ischemia/reperfusion injury (Talukder M. A., et al., Cardiovasc Res 84:345-352 (2009)). Therefore the activation of Mas in cardiomyocytes during ischemia/reperfusion should lead to activation of G_{q}-PLC-IP3-Ca²⁺ signaling, which should contribute to elevated cytosolic and mitochondrial Ca²⁺ loading and thus contribute to cell death by either apoptosis or necrosis. Cardiomyocyte loss by apoptosis has been recognized as a major factor contributing to ischemia/reperfusion injury (Mani K., Heart Fail Rev 13:193-209 (2008)). Indeed, we see marked increases in cardiomyocyte apoptosis in rat hearts after ischemia/reperfusion injury. Our observation that administration of Mas receptor inverse agonists prior to reperfusion reduces cardiomyocyte apoptosis supports this mechanism.

In addition to reducing infarct size, the improved coronary flow and Ca²⁺ handling during reperfusion should result in fewer ventricular arrhythmias. Our observation that a Mas inverse agonist decreased the incidence of ventricular arrhythmias during reperfusion indicates that this is a third mechanism whereby Mas inverse agonists have the potential to provide cardioprotection.

As stated earlier, our data demonstrate that the Mas receptor is expressed in cardiomyocytes and coronary arteries across multiple species including humans. More importantly, our studies have focused on a previously underappreciated aspect of Mas receptor pharmacology; G-protein signaling. We find that the Mas receptor preferentially couples to G_{q} resulting in PLC activation and increased intracellular calcium, and that Gᵢ coupling can also occur at higher drug concentrations (Example 3.3). Our coronary flow studies in isolated hearts support this signaling mechanism. Inhibition of Mas signaling was shown to provide cardioprotection through a mechanism involving improved coronary flow, reduced apoptosis, and reduced in the incidence of arrhythmias. Furthermore, we show that inhibition of Mas receptor G_{q} signaling in the heart protects against ischemia/reperfusion injury in vivo as demonstrated by reduced infarct size (Example 4). Together, these results reveal a previously unrecognized pathological role for excessive Mas-G_{q} signaling in the setting of myocardial ischemia/reperfusion injury, and indicate that inhibition of Mas-G_{q} signaling is therapeutically beneficial.

The standard treatment for myocardial infarction is reperfusion of the ischemic area by thrombolysis or percutaneous coronary angioplasty. Release of the blockage and return of blood flow to the affected area is crucial for heart tissue survival; however, damage beyond that generated by ischemia is typically observed in the reperfused heart tissue. The manifestations of reperfusion injury include arrhythmia, reversible contractile dysfunction-myocardial stunning, endothelial dysfunction and cell death. Currently, there is no effective treatment for reperfusion injury available. Inverse agonists/antagonists of the Mas receptor are cardio-protective. The cardio-protection observed with inhibitors of the Mas receptor is consistent with the inhibition of other myocardial G_{q} coupled receptors, such as the angiotensin AT₁ receptor (De Gasparo, M. et al., Pharmacol Rev 52:415-472 (2000)) and the endothelin receptor ET_{A} (Douglas, S. A. and Ohlstein, E. H. Vascular Research 34:152-164 (1997) and Takigawa, M. et al., Eur. L. Biochem. 228:102-108 (1995)).

Based on expression data, the cellular signaling of the Mas receptor (G_{q}/PLC activation and increased intracellular Ca⁺²), and comparison with similar G_{q} coupled receptors (e.g., AT₁ and ETD, Mas inverse agonists of Formula (I) are useful in the treatment of a number of the conditions, such as, hypertension, recurrence of atrial fibrillation, reduction in the incidence of Alzheimer's disease, progression of Alzheimer's disease, dementia, and other conditions provided herein. Angiotensin AT₁ receptor inhibitors are well known in medicine. Examples of AT₁ receptor inhibitors include candesartan (Atacand™), eprosartan (Teveten™), irbesartan (Avapro™), telmisartan (Micardis™), valsartan (Diovan™), losartan (Cozaar™), and olmesartan (Benicar™). AT₁ receptor inhibitors are useful in the treatment of hypertension (high blood pressure). Persistent hypertension is one of the risk factors for stroke, myocardial infarction, heart failure and arterial aneurysm, and is a leading cause of chronic kidney failure (Pierdomenico, S. D., et al., American J. Hypertension 22:842-847 (2009)). AT₁ receptor inhibitors also prevent/treat the recurrence of atrial fibrillation. In addition, AT₁ receptor inhibitors are associated with a significant reduction in the incidence and progression of Alzheimer's disease and dementia compared with angiotensin converting enzyme inhibitors or other cardiovascular drugs (Li, N.-C. et al., BMJ 2010; 340:b5465).

Inverse agonists and antagonists of the G protein-coupled Mas receptor and pharmaceutical compositions comprising the same are useful in methods of treatment or alleviation of diseases or disorders of the heart, brain, kidney, and reproductive system resulting from ischemia, or reperfusion subsequent to ischemia, and any downstream complication(s) related thereto. The present invention further relates to methods of treatment or alleviation of diseases or disorders of the vasculature resulting from vasoconstriction or hypertension and any downstream complication(s) resulting from elevated blood pressure and/or reduced tissue perfusion. Specifically, the Mas receptor inverse agonists and antagonists are useful in treating diseases or disorders characterized by an active, hyperactive, or an improperly active Mas receptor, and/or in ameliorating the symptoms thereof in a subject in need of such treatment. The methods involve contacting a cell, a tissue, or an organ expressing a Mas receptor of a subject in need of such treatment with an effective amount of an inverse agonist or antagonist of the Mas receptor. The contacted cell, tissue, or organ may be in a patient, or may be isolated from the patient, contacted with the Mas receptor inverse agonist or antagonist, and returned to the patient's body. The Mas receptor inverse agonist or antagonist decreases the activity of, or signaling through, the Mas receptor thereby treating the disease/disorder, reducing the risk of developing the disease/disorder, or alleviating the symptoms of the disease/disorder.

### 1. Regulation of the Vascular System

Mas receptor inverse agonists and antagonists are useful in prophylactic and therapeutic treatments, in part, because of their ability to reduce or inhibit vasoconstriction and/or promote vasodilation. Regulating the vascular system (e.g., by vasodilation and/or vasorelaxation) is helpful in treating conditions where there is a restriction or impediment to normal blood flow, or reducing the symptoms of such conditions. Non-limiting examples of conditions that benefit from vascular regulation using Mas receptor inverse agonists and antagonists are provided below.

a. Heart

The compounds described herein are particularly useful in reducing the likelihood of developing coronary heart disease as well as in the treatment of coronary heart disease and the symptoms thereof. Coronary heart disease, also known as coronary artery disease, is a narrowing of the small blood vessels that supply blood and oxygen to the heart and is the leading cause of death in the United States for men and women. This disease is usually caused by a condition called atherosclerosis, which occurs when fatty material and other substances form a plaque build-up on the walls of the arteries causing them to get narrow. As the coronary arteries narrow, blood flow to the heart can slow down or stop.

A restriction in blood supply can lead to ischemia. Ischemia results in tissue damage because of a lack of oxygen and nutrients. Mas receptor inverse agonists and antagonists are effective in reducing ischemia by reducing vasoconstriction and removing the restriction on blood flow. Thus, compounds of the present invention are useful for providing cardioprotection during and/or following an obstruction or reduced blood flow in the heart.

Ischemia can result in a condition called angina pectoris, more commonly called angina, which is a temporary and often recurring chest pain caused by a lack of or inadequate oxygenated blood feeding the heart muscles. The compounds of the present invention are useful in reducing the risk of angina attacks or the symptoms thereof.

Myocardial infarction, more commonly known as heart attack, occurs when the blood supply to a part of the heart is interrupted by blockage of the coronary blood vessels causing heart cells in that part of the heart to die. The Mas receptor inverse agonists and antagonists are helpful in reducing vasoconstriction thereby reducing the risk of a myocardial infarction. In addition, the inverse agonists and antagonists of the present invention are helpful in promoting vasorelaxation following myocardial infarction.

The no-reflow phenomenon, which usually manifests as ECG changes and chest pain is a failure to restore normal myocardial blood flow despite removal of the coronary obstruction. The no-reflow phenomenon has been shown to complicate thrombolytic therapy and percutaneous revascularization. The compounds of the present invention are useful in the treatment of the no-reflow phenomenon and the symptoms thereof.

Hypertension is a cardiac chronic condition in which the systemic arterial blood pressure is elevated. Persistent hypertension is one of the risk factors for myocardial infarction. Pulmonary hypertension is an increase in blood pressure in the pulmonary artery, pulmonary vein, or pulmonary capillaries, together known as the lung vasculature. Pulmonary hypertension can be a severe disease with a markedly increased risk for heart failure. Mas receptor inverse agonists and antagonists are useful in stabilizing blood pressure and thereby reducing hypertension and also ameliorating the symptoms thereof.

Angioplasty is a catheter-based technique used to open arteries obstructed by a blood clot. Mas receptor inverse agonists and antagonists, by promoting vasodilation, can have the effect of reducing the risk of the formation of blood clots following this procedure.

Coronary bypass surgery is a surgical procedure in which an artery or vein is taken from elsewhere in the body and grafted to a blocked coronary artery, rerouting blood around the blockage and through the newly attached vessel. The compounds of the present invention are helpful in reducing the risk of vasoconstriction following this procedure.

Ischemia/reperfusion Injury is the tissue damage that is caused when blood supply returns to the tissue after a period of ischemia. The absence of oxygen and nutrients from blood creates a condition in which the restoration of circulation results inflammation and oxidative damage through the induction of oxidative stress rather than restoration of normal function. Mas receptor inverse agonists and antagonists are useful in treating reperfusion injury. In some embodiments, the reperfusion injury is injury following cardioplegia. In some embodiments, the reperfusion injury is injury following angioplasty.

b. Brain

A transient ischemic attack or mini-stroke is a brief interruption of blood flow to the brain caused by an obstruction to blood flow. Example 10 shows that inhibiting Mas receptor signaling reduced brain damage associated with transient ischemic stroke. Thus, Mas receptor inverse agonists and antagonists are helpful in treating transient ischemic attack and the symptoms thereof.

A stroke is an event in which the brain does not receive adequate amounts of oxygenated blood and is usually caused by ischemia (resulting from blockage of a cerebral blood vessel) or a hemorrhage. The compounds of the present invention are useful in treating strokes and the symptoms thereof. In addition, these compounds are useful in reducing the risk of the reoccurrence of a stroke or a mini stroke.

Thus, compounds of the present invention are useful for providing neuroprotection during and/or following an obstruction or reduced blood flow in the brain and in the treatment of one or more of the following Mas receptor-mediated disorders: stroke, brain attack, neuroprotection, brain ischemia (thrombotic, embolic and hypoperfusion), focal or multifocal brain ischemia, global brain ischemia, ischemic brain injury, acute ischemic brain damage, acute ischemic brain injury, brain infarction, brain reperfusion injury, brain hypoxia, cerebral reperfusion injury, neuronal reperfusion injury, ischemic neurological disorders, ischemic brain damage, cerebral hypoxia, cerebral ischemia, cerebral ischemic injury, hypoxic-ischemic brain injury, anoxic brain injury, anoxic brain damage, anoxic encephalopathy, subcortical ischemic depression, moyamoya disease, and cardiorespiratory arrest.

c. Reproductive System

Erectile dysfunction is the inability of a male subject to develop or maintain penile erection for normal sexual performance. A penile erection is the hydraulic effect of blood entering and being retained in the corpus cavernosa, which are sponge-like bodies within the penis. Erectile dysfunction is indicated when an erection is difficult to produce. The Mas receptor is expressed in the corpus cavernosa and the vasodilatory properties of the inverse agonists and antagonists of the Mas receptor make them useful in treating erectile dysfunction.

d. Intestine

Both the large and small bowel can also be affected by ischemia. Ischemic colitis is a medical condition in which inflammation and injury of the large intestine result from inadequate blood supply usually caused by changes in the systemic circulation (e.g. low blood pressure) or local factors such as constriction of blood vessels or a blood clot. Ischemia of the small bowel is called mesenteric ischemia. The compounds of the present invention are useful in reducing ischemia of both the large and small intestine.

e. Limbs—Peripheral Vascular Disease

Acute limb ischemia is caused by the lack of, or reduced, blood flow to a limb. It is usually due to either an embolism or thrombosis of an artery in subjects with underlying peripheral vascular disease. A blockage in the legs can lead to leg pain or cramps with activity (claudication), changes in skin color, sores or ulcers, and feeling tired in the legs. Total loss of circulation can lead to gangrene and loss of a limb. Mas receptor inverse agonists and antagonists can improve blood flow thereby treating the risk of developing acute limb ischemia in subjects in need thereof.

f. Kidney

Renal artery stenosis is a decrease in the diameter of the renal arteries. The resulting restriction of blood flow to the kidneys may lead to impaired kidney function and high blood pressure, referred to as renovascular hypertension (RVHT). Renal artery stenosis is a major cause of RVHT and accounts for 1%-10% of the roughly 50 million cases of hypertension in the United States. Renovascular hypertension occurs when the artery to one of the kidneys is narrowed, while renal failure occurs when the arteries to both kidneys are narrowed. The decreased blood flow to both kidneys increasingly impairs renal function. Example 9 demonstrates that Mas inverse agonists can be protective for kidney function following ischemia reperfusion injury. Thus, the compounds of the present invention are useful in improving blood flow to and within the kidneys. In addition, Mas receptor inverse agonists and antagonists are helpful in treating or reducing the risk of developing renal artery stenosis, renovascular hypertension, and renal failure. Furthermore, the compounds described herein are also useful in treating chronic kidney disease and diabetic nephropathy and the symptoms thereof.

Thus, compounds of the present invention are useful for providing renoprotection/renal protection during and/or following an obstruction or reduced blood flow in the kidney and in the treatment of one or more of the following Mas receptor-mediated disorders: nephropathy, nephrotic syndrome, obstruction nephropathy, obstructive nephropathy, diabetic nephropathy, renal hypertension, renovascular hypertension, renal ischemia, renal ischemic injury, renal ischemia-reperfusion injury, renal reperfusion injury, acute renal injury, acute kidney injury, acute renal failure, acute kidney failure, acute tubular necrosis, contrast nephropathy, chronic kidney disease, chronic renal failure, chronic renal insufficiency, end stage renal disease, end stage renal failure, focal segmental glomerulosclerosis, glomerulonephritis, diabetes and diabetic kidney disease, diabetes insipidus, Fabry's disease, focal segmental glomerulosclerosis, focal sclerosis, focal glomerulosclerosis, Gitelman syndrome, glomerular diseases, high blood pressure and kidney disease, IgA nephropathy (Berger's disease), interstitial nephritis, lupus, malignant hypertension, microscopic polyangiitis (MPA), preeclampsia, polyarteritis, proteinuria, renal artery stenosis, renal infarction, reflux nephropathy, scleroderma renal crisis, tuberous sclerosis, and warfarin-related nephropathy.

### 2. Inhibiting Calcium Signaling

Mas receptor inverse agonists and antagonists are also useful in prophylactic and therapeutic treatments, in part, because of their ability to reduce or inhibit calcium signaling in cells or correct improper calcium handling by cells.

The Mas receptor is a G_{q} coupled receptor. Stimulation of the Mas receptor leads to the release of calcium stored within intracellular compartments. Contractility of cardiac myocytes is regulated by changes in intracellular calcium concentration. Improper calcium handling by cardiac myocytes can lead to inappropriate contractile activity. In addition, inappropriate calcium release from intracellular compartments can result in conditions such as cardiac arrhythmias, pathological structural changes in the myocardium, and apoptosis. Inverse agonists and antagonists of the Mas receptor are useful in treating any disease or disorder arising from improper regulation of calcium signaling and/or handling by cells, or the symptoms thereof.

a. Arrhythmias

An arrhythmia is a problem with the rate or rhythm of the heartbeat. During an arrhythmia, the heart can beat too fast, too slow, or with an irregular rhythm. A heartbeat that is too fast is called tachycardia. A heartbeat that is too slow is called bradycardia. When the heart rate is too fast, too slow, or irregular, the heart may not be able to pump enough blood to the body. Lack of blood flow can damage the brain, heart, and other organs. There are several known types of arrhythmias such as supraventricular arrhythmias, ventricular arrhythmias, and bradyarrhythmias.

Supraventricular arrhythmias are tachycardias that start in the atria or the atrioventricular node and include atrial fibrillation, atrial flutter, paroxysmal supraventricular tachycardia, and Wolff-Parkinson-White syndrome. Atrial fibrillation, a condition which affects roughly two million Americans each year, is the most common type of arrhythmia. In this condition, the atria (the upper chambers of the heart) rapidly fire electrical signals that cause them to quiver rather than contract normally. The result is an abnormally fast and highly irregular heartbeat.

Ventricular arrhythmias are abnormal rapid heart rhythms that originate in the ventricles (the lower chambers of the heart). Ventricular arrhythmias include ventricular tachycardia and ventricular fibrillation, both of which are life-threatening arrhythmias most commonly associated with heart attacks.

Bradyarrhythmias are arrhythmias in which the heart rate is slower than normal. If the heart rate is too slow, not enough blood reaches the brain.

Reperfusion of the ischemic myocardium may play an important role in the genesis of life-threatening reperfusion arrhythmias. Reflow may occur as a result of abrupt cessation of coronary artery spasm or upon dislodgment of platelet aggregates with the attendant washout of products of cellular ischemia and the released substances exert a transient but potent arrhythmogenic effect resulting in reperfusion arrhythmias.

The Mas receptor inverse agonists and antagonists are effective in treating and/or reducing the likelihood of developing arrhythmias and are also effective in treating the symptoms of arrhythmias.

b. Apoptosis

Apoptosis is an important component of normal development as well as the pathogenesis of several diseases including cardiovascular diseases. Calcium levels play a key role in apoptosis of cardiomyocytes. Signaling through the Mas receptor mobilizes calcium stores and triggers apoptosis by elevation of intracellular fee calcium. Inverse agonists and antagonists of the Mas receptor are useful in protecting the myocardium from cell death.

### 3. Inflammatory Disorders, Autoimmune Disorders, and Associated Conditions

Inflammation is a complex biological response of vascular tissues to harmful stimuli, such as pathogens, damaged cells, or irritants. While after injury or in certain conditions inflammation is a normal, healthy response, inflammation that results in the immune system attacking the body's own cells or tissues may also cause abnormal inflammation, which results in chronic pain, redness, swelling, stiffness, and damage to normal tissues. Prolonged inflammation, known as chronic inflammation, can lead to a host of diseases, such as hay fever, periodontitis, atherosclerosis, rheumatoid arthritis, and even cancer (e.g., gallbladder carcinoma). Mas receptor inverse agonists and antagonists such as those described herein are useful in the treatment and/or prevention of inflammatory disorders as well as conditions associated with inflammation.

Inflammatory disorders are generally associated with elevated levels of certain cytokines. Cytokines include interleukins (IL), interferons (IFNs), chemokines (proteins that direct white blood cells to move to sites of inflammation), tumor necrosis factors (TNFs), and colony stimulating factors (CSFs). The cytokines associated with increased inflammation are called “proinflammatory cytokines” and include IL-1α, IL-1β, IL-2, IL-6, IL-10, IL-12, IL-15, IL-18, TNFα, secreted lymphotoxin α(TNFβ), lymphotoxin β, IFNα, IFNβ, IFNγ, GM-CSF, M-CSF, lymphotoxin αβ, LIGHT, CD40 ligand, Fas ligand, CD30 ligand, CD27 ligand, 4-1BB ligand, the Ox40 ligand, TRAIL, TWEAK, TRAMP, CXC chemokines (e.g., L-8, GRO-α, GRO-β, PF-4, IP-10, and Mig), and CC chemokines (e.g., eotaxin, eotaxin-2, and MCP-4). The compounds of the present invention are useful in treating or preventing inflammation, at least in part, by reducing the levels of such proinflammatory cytokines.

For example, the compounds described herein can be used to treat inflammatory disorders such as, those mediated by tumor necrosis factor-α (TNFα). Mas receptor gene expression correlates with TNFα expression by inflammatory immune cells, such macrophages (Example 6). TNFα is a cytokine that has been identified as a mediator of immunity, of inflammation, of cell proliferation, and of fibrosis. This mediator is present in large quantities in inflamed synovial tissues and plays an important role in the pathogenesis of autoimmunity (Black et, al., Annu. Rep. Med. Chem., 32:241-250 (1997)). Elevated levels of TNFα levels have been associated with many inflammatory diseases such as sepsis and rheumatoid arthritis. Rheumatoid arthritis is a chronic inflammatory disorder that affects multiple peripheral joints. Over expression of TNFα and other proinflammatory cytokines has been observed in patients with arthritis (Feldmann et. al., Prog Growth Factor Res 4:247-55 (1992)). Furthermore, transgenic animals that over express human TNFα develop an erosive polyarthritis with many characteristics associated with the disease (Keffer et. al., EMBO J. 10(13):4025-31 (1991)). The success of anti-TNFα antibody therapy (Rituximab) has transformed the management of the disease (Edwards, et. al., N. Engl. J. Med., 350(25): 2572-81 (2004)). It has now been found that compounds of the present invention are capable of decreasing levels of TNFα, see Example 7, and have also been shown in a well-known animal model of inflammation (the carrageenan-induced inflammation paw swelling model) to be effective in reducing inflammation, see Example 8. Given the ability of the compounds of the present invention to reduce TNFα levels, the Mas receptor inverse agonists and antagonists are beneficial in treating TNFα-related disorders such as, but not limited to:

(A) acute and chronic immune and autoimmune pathologies, such as systemic lupus erythematosus (SLE), rheumatoid arthritis, juvenile rheumatoid arthritis, psoriatic arthritis, osteoarthritis, refractory rheumatoid arthritis, chronic non-rheumatoid arthritis, osteoporosis/bone resorption, thyroidosis, graft versus host disease, scleroderma, diabetes mellitus, Graves' disease, and the like;

(B) infections, including, but not limited to, sepsis syndrome, cachexia, septic shock, endotoxic shock, circulatory collapse and shock resulting from acute or chronic bacterial infection, acute and chronic parasitic and/or infectious diseases, bacterial, fungal, or viral such as AIDS (including symptoms of cachexia, autoimmune disorders, AIDS dementia complex and infections);

(C) inflammatory diseases, such as chronic inflammatory pathologies and vascular inflammatory pathologies, including chronic inflammatory pathologies such as sarcoidosis, inflammatory bowel disease, ulcerative colitis, Crohn's disease, systemic sclerosis, psoriasis, dermatomyositis, polyomyositis, and vascular inflammatory pathologies, such as, but not limited to, disseminated intravascular coagulation, atherosclerosis, and Kawasaki's pathology;

(D) neurodegenerative diseases, including, but not limited to, demyelinating diseases, such as multiple sclerosis and acute transverse myelitis; extrapyramidal and cerebellar disorders such as lesions of the corticospinal system; disorders of the basal ganglia or cerebellar disorders; hyperkinetic movement disorders such as Huntington's chorea and senile chorea; drug-induced movement disorders, such as those induced by drugs which block CNS dopamine receptors; hypokinetic movement disorders, such as Parkinson's disease; progressive supranucleo palsy; cerebellar and spinocerebellar disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and Machado-Joseph); and systemic disorders (Refsum's disease, abetalipoprotemia, ataxia, telangiectasia, and mitochondrial multi-system disorder); demyelinating core disorders, such as multiple sclerosis, acute transverse myelitis; disorders of the motor unit, such as neurogenic muscular atrophies (anterior horn cell degeneration, such as amyotrophic lateral sclerosis, infantile spinal muscular atrophy and juvenile spinal muscular atrophy), Alzheimer's disease, Down's syndrome in middle age, diffuse Lewy body disease, senile dementia of Lewy body type, Wernicke-Korsakoff syndrome, chronic alcoholism, Creutzfeldt-Jakob disease, subacute sclerosing panencephalitis, Hallerrorden-Spatz disease, and dementia pugilistica, or any subset thereof;

(E) malignant pathologies involving TNF-secreting tumors or other malignancies involving TNF, such as, but not limited to leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or mycosis fungoides)); and

(F) alcohol-induced hepatitis.

The compounds of the invention are equally useful in treating and/or preventing IL-1 related disorders. In certain embodiments, the IL-1 related disorder includes (a) inflammatory diseases such as osteoarthritis, pancreatitis and asthma; (b) autoimmune diseases such as glomerular nephritis, rheumatoid arthritis, scleroderma, and alphosis; and (c) infectious diseases such as septicemia and septic shock.

The cytokine IL-6 acts as a proinflammatory cytokine in part through its effects on TNFα and IL-1. Thus, the compounds of the present invention are also useful in treating IL-6 related disorders such as autoimmune diseases and chronic inflammatory proliferative diseases. In specific embodiments, the compounds of the present invention are useful in treating and/or preventing rheumatoid arthritis, systemic-onset juvenile chronic arthritis, osteoporosis, psoriasis, diabetes, atherosclerosis, depression, Alzheimer's disease, systemic lupus erythematosus, and prostate cancer.

The compounds of the present invention are also useful to treat and/or prevent disorders associated with deregulated expression and/or activity of other proinflammatory cytokines such as IL-2, IL-10, IL-12, IL-15, IL-18, TNFβ, lymphotoxin β, IFNα, IFNβ, IFNγ, GM-CSF, M-CSF, lymphotoxin αβ, LIGHT, CD40 ligand, Fas ligand, CD30 ligand, CD27 ligand, 4-1BB ligand, the Ox40 ligand, TRAIL, TWEAK, TRAMP, CXC chemokines (e.g., L-8, GRO-α, GRO-β, PF-4, IP-10, and Mig), and CC chemokines (e.g., eotaxin, eotaxin-2, and MCP-4).

Mas receptor inverse agonists and antagonists such as those described herein are useful in the treatment and/or prevention of autoimmune and autoinflammatory disorders. An autoimmune disorder is a condition that occurs when the immune system mistakenly attacks and destroys healthy body tissue. An autoimmune disorder may result in the destruction of one or more types of body tissue; abnormal growth of an organ; and/or changes in organ function. Autoinflammatory diseases are a relatively new category of diseases that are different from autoimmune diseases. However, autoimmune and autoinflammatory diseases share common characteristics in that both groups of disorders result from the immune system attacking the body's own tissues, and also result in increased inflammation.

The compounds of the present invention are useful in the treatment of one or more of the following inflammatory disorders, autoimmune disorders, and/or disorders related to inflammatory or autoimmune diseases: acne vulgaris, adult respiratory distress syndrome, allergy, allergic asthma, Alzheimer's disease, amyloidosis, ankylosing spondylitis, asthma, bronchopulmonary aspergillosis, allergic rhinitis, autoimmune hemolytic anemia, acanthosis nigricans, allergic contact dermatitis, Addison's disease, atopic dermatitis, alopecia greata, alopecia universalis, amyloidosis, anaphylactoid purpura, anaphylactoid reaction, aplastic anemia, hereditary angioedema, idiopathic angioedema, cranial arteritis, giant cell arteritis, Takayasu's arteritis, temporal arteritis, asthma, autoimmune oophoritis, autoimmune orchitis, autoimmune polyendocrine failure, bacterial septic shock, bacterial toxic shock, Behcet's disease, Berger's disease, Buerger's disease, bronchitis, bullous pemphigus, chronic mucocutaneous candidiasis, chronic grafts versus host disease, Caplan's syndrome, post-myocardial infarction syndrome, post-pericardiotomy syndrome, carditis, celiac disease, celiac sprue; Chagas disease, Chediak-Higashi syndrome, Churg-Strauss disease, chronic recurrent uveitis, Cogan's syndrome, cold agglutinin disease, CREST syndrome, Crohn's disease, cryoglobulinemia, cryptogenic fibrosing alveolitis, delayed type hypersensitivity disorders, dermatitis herpetifomis, dermatomyositis, juvenile dermatomyositis, diabetes mellitus, Diamond-Blackfan syndrome, DiGeorge syndrome, discoid lupus erythematosus, endometriosis, eosinophilic fasciitis, episcleritis, drythema elevatum diutinum, erythema marginatum, erythema multiforme, erythema nodosum, familial amyloid polyneuropathies, familial Mediterranean fever, Felty's syndrome, pulmonary fibrosis, anaphylactoid glomerulonephritis, autoimmune glomerulonephritis, post-streptococcal glomerulonephritis, post-transplantation glomerulonephritis, membranous glomerulopathy, Goodpasture's syndrome, immune-mediated granulocytopenia, graft versus host disease, granuloma annulare, allergic granulomatosis, granulomatous myositis, Grave's disease, Hashimoto's thyroiditis, hemolytic disease of the newborn, idiopathic hemochromatosis, Henoch-Schoenlein purpura, chronic active and chronic progressive hepatitis, histiocytosis X, hypereosinophilic syndrome, hypersensitivities, idiopathic thrombocytopenic purpura, immune deficiency, common variable immunodeficiency, interstitial cystitis, Job's syndrome, juvenile rheumatoid arthritis (juvenile chronic arthritis), Kawasaki's disease, keratitis, keratoconjunctivitis sicca, Landry-Guillain-Barre-Strohl syndrome, lepromatous leprosy, Loeffler's syndrome, lupus, Lyell's syndrome, lyme disease, lymphomatoid granulomatosis, lymphoproliferative disease, malaria, meningitis, systemic mastocytosis, mixed connective tissue disease, mononeuritis multiplex, Muckle-Wells syndrome, mucocutaneous lymph node syndrome, mucocutaneous lymph node syndrome, multicentric reticulohistiocytosis, multiple sclerosis, myasthenia gravis, mycosis fungoides, myeloproliferative disorder, nephrotic syndrome, ovarian cancer, recurrent ovarian cancer, overlap syndrome, panniculitis, paroxysmal cold hemoglobinuria, paroxysmal nocturnal hemoglobinuria, pelvic inflammatory diseases, pemphigoid, pemphigus, pemphigus erythematosus, pemphigus foliaceus, pemphigus vulgaris, pigeon breeder's disease, plasmacytoma, pneumonitis, polyarteritis nodosa, refractory multiple myeloma, rheumatic polymyalgia, polymyositis, diopathic polyneuritis, preeclampsia/eclampsia, primary biliary cirrhosis, systemic sclerosis, progressive systemic sclerosis (scleroderma), multiple sclerosis, psoriasis, psoriatic arthritis, pulmonary alveolar proteinosis, pulmonary fibrosis, Raynaud's phenomenon/syndrome, Reidel's thyroiditis, Reiter's syndrome, relapsing polychrondritis, rheumatic fever, rheumatoid arthritis, sarcoidosis, scleritis, sclerosing cholangitis, serum sickness, Sezary syndrome, Sjogren's syndrome, Stevens-Johnson syndrome, Still's disease, subacute sclerosing panencephalitis, sympathetic ophthalmia, systemic lupus erythematosus, transplant rejection, tumor proliferation and metastasis, ulcerative colitis, undifferentiated connective tissue disease, chronic urticaria, cold urticaria, uveitis, vasculitis, systemic necrotizing vasculitis, viral replication in AIDS, vitiligo, Weber-Christian disease, Wegener's granulomatosis, and Wiskott-Aldrich syndrome.

One aspect of the present invention pertains to methods selected from one or more of the following for: 1) the treatment of a Mas receptor-mediated disorder in an individual; 2) the treatment of a disorder alleviated by vasodilation in an individual; 3) the treatment of a disorder alleviated by vasorelaxation in an individual; 4) the treatment of a disorder alleviated by inhibiting vasoconstriction in an individual; 5) the treatment of a disorder alleviated by promoting normal blood flow in an individual; 6) the treatment of the formation of blood clots following angioplasty in an individual; 7) reducing injury due to blood clot formation in an individual; 8) reducing injury due to blood clot formation following angioplasty in an individual; 9) the treatment of vasoconstriction following coronary bypass surgery in an individual; 10) the treatment of ischemia reperfusion injury during and/or following coronary bypass surgery in an individual; 11) the treatment of ischemia reperfusion myocardial injury during and/or following coronary bypass surgery in an individual; 12) the treatment of a disorder alleviated by inhibiting calcium signaling in cells in an individual; 13) the treatment of a disorder alleviated by correcting improper calcium handling by cells in an individual; 14) the treatment of arrhythmia in an individual; 15) the treatment of ischemia reperfusion-induced arrhythmia in an individual; 16) the treatment of reperfusion-induced myocardial injury in an individual; 17) the treatment of reperfusion-induced cardiomyocyte injury in an individual; 18) the treatment of reperfusion-induced cardiomyocyte cell death in an individual; 19) the treatment of an inflammatory disorder in an individual; 20) for providing neuroprotection in an individual; and 21) for providing renal protection in an individual; comprising administering to the individual in need thereof or prescribing to the individual in need thereof, a therapeutically effective amount of: A) a compound of the present invention; B) a crystalline form of the present invention; C) compositions of the present invention; D) a pharmaceutical product of the present invention; or E) a pharmaceutical composition of the present invention; each optionally in combination with a therapeutically effective amount of a second pharmaceutical agent.

One aspect of the present invention pertains to the use of: A) a compound of the present invention; B) a crystalline form of the present invention; or C) compositions of the present invention; each optionally in combination with a second pharmaceutical agent, in the manufacture of a medicament, selected from one or more of the following: 1) the treatment of a Mas receptor-mediated disorder in an individual; 2) the treatment of a disorder alleviated by vasodilation in an individual; 3) the treatment of a disorder alleviated by vasorelaxation in an individual; 4) the treatment of a disorder alleviated by inhibiting vasoconstriction in an individual; 5) the treatment of a disorder alleviated by promoting normal blood flow in an individual; 6) the treatment of the formation of blood clots following angioplasty in an individual; 7) reducing injury due to blood clot formation in an individual; 8) reducing injury due to blood clot formation following angioplasty in an individual; 9) the treatment of vasoconstriction following coronary bypass surgery in an individual; 10) the treatment of ischemia reperfusion injury during and/or following coronary bypass surgery in an individual; 11) the treatment of ischemia reperfusion myocardial injury during and/or following coronary bypass surgery in an individual; 12) the treatment of a disorder alleviated by inhibiting calcium signaling in cells in an individual; 13) the treatment of a disorder alleviated by correcting improper calcium handling by cells in an individual; 14) the treatment of arrhythmia in an individual; 15) the treatment of ischemia reperfusion-induced arrhythmia in an individual; 16) the treatment of reperfusion-induced myocardial injury in an individual; 17) the treatment of reperfusion-induced cardiomyocyte injury in an individual; 18) the treatment of reperfusion-induced cardiomyocyte cell death in an individual; 19) the treatment of an inflammatory disorder in an individual; 20) for providing neuroprotection in an individual; and 21) for providing renal protection in an individual.

One aspect of the present invention pertains to: A) compounds of the present invention; B) crystalline forms of the present invention; C) compositions of the present invention; D) pharmaceutical products of the present invention; or E) pharmaceutical compositions of the present invention; each optionally in combination with a second pharmaceutical agent, for use in a method of treatment of the human or animal body by therapy.

One aspect of the present invention pertains to: A) compounds of the present invention; B) crystalline forms of the present invention; C) compositions of the present invention; D) pharmaceutical products of the present invention; or E) pharmaceutical compositions of the present invention; each optionally in combination with a second pharmaceutical agent, for use in a method of treatment of/for, selected from one or more for the following: 1) the treatment of a Mas receptor-mediated disorder in an individual; 2) the treatment of a disorder alleviated by vasodilation in an individual; 3) the treatment of a disorder alleviated by vasorelaxation in an individual; 4) the treatment of a disorder alleviated by inhibiting vasoconstriction in an individual; 5) the treatment of a disorder alleviated by promoting normal blood flow in an individual; 6) the treatment of the formation of blood clots following angioplasty in an individual; 7) reducing injury due to blood clot formation in an individual; 8) reducing injury due to blood clot formation following angioplasty in an individual; 9) the treatment of vasoconstriction following coronary bypass surgery in an individual; 10) the treatment of ischemia reperfusion injury during and/or following coronary bypass surgery in an individual; 11) the treatment of ischemia reperfusion myocardial injury during and/or following coronary bypass surgery in an individual; 12) the treatment of a disorder alleviated by inhibiting calcium signaling in cells in an individual; 13) the treatment of a disorder alleviated by correcting improper calcium handling by cells in an individual; 14) the treatment of arrhythmia in an individual; 15) the treatment of ischemia reperfusion-induced arrhythmia in an individual; 16) the treatment of reperfusion-induced myocardial injury in an individual; 17) the treatment of reperfusion-induced cardiomyocyte injury in an individual; 18) the treatment of reperfusion-induced cardiomyocyte cell death in an individual; 19) the treatment of an inflammatory disorder in an individual; 20) for providing neuroprotection in an individual; and 21) for providing renal protection in an individual.

One aspect of the present invention pertains to the use of a pharmaceutical agent in combination with: A) a compound of the present invention; B) a crystalline form of the present invention; or C) compositions of the present invention; in the manufacture of a medicament, selected from one or more of the following: 1) the treatment of a Mas receptor-mediated disorder in an individual; 2) the treatment of a disorder alleviated by vasodilation in an individual; 3) the treatment of a disorder alleviated by vasorelaxation in an individual; 4) the treatment of a disorder alleviated by inhibiting vasoconstriction in an individual; 5) the treatment of a disorder alleviated by promoting normal blood flow in an individual; 6) the treatment of the formation of blood clots following angioplasty in an individual; 7) reducing injury due to blood clot formation in an individual; 8) reducing injury due to blood clot formation following angioplasty in an individual; 9) the treatment of vasoconstriction following coronary bypass surgery in an individual; 10) the treatment of ischemia reperfusion injury during and/or following coronary bypass surgery in an individual; 11) the treatment of ischemia reperfusion myocardial injury during and/or following coronary bypass surgery in an individual; 12) the treatment of a disorder alleviated by inhibiting calcium signaling in cells in an individual; 13) the treatment of a disorder alleviated by correcting improper calcium handling by cells in an individual; 14) the treatment of arrhythmia in an individual; 15) the treatment of ischemia reperfusion-induced arrhythmia in an individual; 16) the treatment of reperfusion-induced myocardial injury in an individual; 17) the treatment of reperfusion-induced cardiomyocyte injury in an individual; 18) the treatment of reperfusion-induced cardiomyocyte cell death in an individual; 19) the treatment of an inflammatory disorder in an individual; 20) for providing neuroprotection in an individual; and 21) for providing renal protection in an individual.

One aspect of the present invention pertains to pharmaceutical agents in combination with: A) a compound of the present invention; B) a crystalline form of the present invention; C) a composition of the present invention; D) a pharmaceutical product of the present invention; or E) a pharmaceutical composition of the present invention; for use in a method of treatment of the human or animal body by therapy.

One aspect of the present invention pertains to pharmaceutical agents in combination with: A) a compound of the present invention; B) a crystalline form of the present invention; C) compositions of the present invention; D) a pharmaceutical product of the present invention; or E) a pharmaceutical composition of the present invention; for use in a method of treatment of/for, selected from one or more for the following: 1) the treatment of a Mas receptor-mediated disorder in an individual; 2) the treatment of a disorder alleviated by vasodilation in an individual; 3) the treatment of a disorder alleviated by vasorelaxation in an individual; 4) the treatment of a disorder alleviated by inhibiting vasoconstriction in an individual; 5) the treatment of a disorder alleviated by promoting normal blood flow in an individual; 6) the treatment of the formation of blood clots following angioplasty in an individual; 7) reducing injury due to blood clot formation in an individual; 8) reducing injury due to blood clot formation following angioplasty in an individual; 9) the treatment of vasoconstriction following coronary bypass surgery in an individual; 10) the treatment of ischemia reperfusion injury during and/or following coronary bypass surgery in an individual; 11) the treatment of ischemia reperfusion myocardial injury during and/or following coronary bypass surgery in an individual; 12) the treatment of a disorder alleviated by inhibiting calcium signaling in cells in an individual; 13) the treatment of a disorder alleviated by correcting improper calcium handling by cells in an individual; 14) the treatment of arrhythmia in an individual; 15) the treatment of ischemia reperfusion-induced arrhythmia in an individual; 16) the treatment of reperfusion-induced myocardial injury in an individual; 17) the treatment of reperfusion-induced cardiomyocyte injury in an individual; 18) the treatment of reperfusion-induced cardiomyocyte cell death in an individual; 19) the treatment of an inflammatory disorder in an individual; 20) for providing neuroprotection in an individual; and 21) for providing renal protection in an individual.

One aspect of the present invention pertains to a pharmaceutical product selected from: a pharmaceutical composition, a formulation, a unit dosage form, and a kit; each comprising: A) a compound of the present invention; B) a crystalline form of the present invention; or C) compositions of the present invention; in combination with a second pharmaceutical agent; for use in a method of treatment of the human or animal body by therapy.

One aspect of the present invention pertains to a pharmaceutical product selected from: a pharmaceutical composition, a formulation, a unit dosage form, and a kit; each comprising: A) a compound of the present invention; B) a crystalline form of the present invention; or C) compositions of the present invention; in combination with a second pharmaceutical agent; for use in a method of treatment of/for, selected from one or more for the following: 1) the treatment of a Mas receptor-mediated disorder in an individual; 2) the treatment of a disorder alleviated by vasodilation in an individual; 3) the treatment of a disorder alleviated by vasorelaxation in an individual; 4) the treatment of a disorder alleviated by inhibiting vasoconstriction in an individual; 5) the treatment of a disorder alleviated by promoting normal blood flow in an individual; 6) the treatment of the formation of blood clots following angioplasty in an individual; 7) reducing injury due to blood clot formation in an individual; 8) reducing injury due to blood clot formation following angioplasty in an individual; 9) the treatment of vasoconstriction following coronary bypass surgery in an individual; 10) the treatment of ischemia reperfusion injury during and/or following coronary bypass surgery in an individual; 11) the treatment of ischemia reperfusion myocardial injury during and/or following coronary bypass surgery in an individual; 12) the treatment of a disorder alleviated by inhibiting calcium signaling in cells in an individual; 13) the treatment of a disorder alleviated by correcting improper calcium handling by cells in an individual; 14) the treatment of arrhythmia in an individual; 15) the treatment of ischemia reperfusion-induced arrhythmia in an individual; 16) the treatment of reperfusion-induced myocardial injury in an individual; 17) the treatment of reperfusion-induced cardiomyocyte injury in an individual; 18) the treatment of reperfusion-induced cardiomyocyte cell death in an individual; 19) the treatment of an inflammatory disorder in an individual; 20) for providing neuroprotection in an individual; and 21) for providing renal protection in an individual.

One aspect of the present invention pertains to pharmaceutical products of the present invention; methods of the present invention; or pharmaceutical agents of the present invention; wherein the pharmaceutical product comprises a pharmaceutical composition. In some embodiments, the pharmaceutical product comprises a formulation. In some embodiments, the pharmaceutical product comprises a unit dosage form. In some embodiments, the pharmaceutical product comprises a kit. In some embodiments, the pharmaceutical product comprises a combined preparation. In some embodiments, the pharmaceutical product comprises a twin pack.

One aspect of the present invention pertains to methods of the present invention; uses of the present invention; compounds of the present invention; crystalline forms of the present invention; compositions of the present invention; pharmaceutical products of the present invention; pharmaceutical compositions of the present invention; or pharmaceutical agents of the present invention; wherein the compound or the crystalline form, and the pharmaceutical agent or the second pharmaceutical agent are administered simultaneously, separately, or sequentially. In some embodiments, the compound or the crystalline form, and the pharmaceutical agent or the second pharmaceutical agent are administered simultaneously. In some embodiments, the compound or the crystalline form, and the pharmaceutical agent or the second pharmaceutical agent are administered separately. In some embodiments, the compound or the crystalline form, and the pharmaceutical agent or the second pharmaceutical agent are administered sequentially.

### Certain Combination Therapies and Pharmaceutical Agents Related Thereto

The inverse agonists and antagonists described herein can be combined with one or more agents that are known to be useful in the treatment of the condition being treated. These agents may be formulated for administration to the subject as a single pharmaceutical composition with the Mas receptor inverse agonists and antagonists of the present invention, or may be formulated as separate compositions. These compositions may be administered to the subject separately, simultaneously, or sequentially.

Therefore, another aspect of the present invention includes methods of treatment or alleviation of diseases or disorders of the heart, brain, kidney, and reproductive system among others and/or in ameliorating the symptoms thereof, comprising administering to an individual in need thereof a therapeutically effective amount of a compound of Formula (I) or a pharmaceutically acceptable salt, solvate, or hydrate thereof, in combination with one or more additional pharmaceutical agents, as described herein.

One aspect of the present invention relates to compositions of the present invention; methods of the present invention; pharmaceutical products of the present invention; pharmaceutical compositions of the present invention; uses of the present invention; compounds of the present invention; crystalline forms of the present invention; or pharmaceutical agents of the present invention; wherein the pharmaceutical agent or the second pharmaceutical agent is selected from: an ACE inhibitor, a beta blocker, a calcium channel blocker, a diuretic, a nitrate, a statin, aspirin, an anti-platelet, adenosine, an endothelin receptor antagonist, a PDE5 inhibitor, an anti-TNF agent (i.e., an agent that inhibits the activity of TNF), and a cardioplegic solution.

In some embodiments, the pharmaceutical agent or the second pharmaceutical agent is an ACE inhibitor. In some embodiments, the pharmaceutical agent or the second pharmaceutical agent is a beta blocker. In some embodiments, the pharmaceutical agent or the second pharmaceutical agent is a calcium channel blocker. In some embodiments, the pharmaceutical agent or the second pharmaceutical agent is a diuretic. In some embodiments, the pharmaceutical agent or the second pharmaceutical agent is a nitrate. In some embodiments, the pharmaceutical agent or the second pharmaceutical agent is a statin. In some embodiments, the pharmaceutical agent or the second pharmaceutical agent is aspirin. In some embodiments, the pharmaceutical agent or the second pharmaceutical agent is an anti-platelet. In some embodiments, the pharmaceutical agent or the second pharmaceutical agent is adenosine. In some embodiments, the pharmaceutical agent or the second pharmaceutical agent is an endothelin receptor antagonist. In some embodiments, the pharmaceutical agent or the second pharmaceutical agent is a PDE5 inhibitor. In some embodiments, the pharmaceutical agent or the second pharmaceutical agent is an anti-TNF agent. In some embodiments, the pharmaceutical agent or the second pharmaceutical agent is a cardioplegic solution.

Non-limiting examples of ACE inhibitors include captopril, zofenopril, enalapril, ramipril, quinapril, perindopril, lisinopril, benazepril, fosinopril, casokinins, lactokinins, and the lactotripeptides Val-Pro-Pro and Ile-Pro-Pro, for example lactotripeptides produced by the probiotic Lactobacillus helveticus or derived from casein. In some embodiments, the pharmaceutical agent or the second pharmaceutical agent is an ACE inhibitor selected from: captopril, zofenopril, enalapril, ramipril, quinapril, perindopril, lisinopril, benazepril, fosinopril, casokinins, lactokinins, Val-Pro-Pro, and Ile-Pro-Pro.

Non-limiting examples of beta-blockers include non-selective agents such as:

alprenolol, bucindolol, carteolol, carvedilol, labetalol, nadolol, penbutolol, pindolol, propranolol, sotalol, and timolol; β1-selective agents such as: acebutolol, atenolol, betaxolol, bisoprolol, celiprolol, esmolol, metoprolol, and nebivolol; β2-selective agents such as: butaxamine and (2R,3R)-3-(isopropylamino)-1-(7-methyl-2,3-dihydro-1H-inden-4-yloxy)butan-2-ol (ICI-118,551); and β3-selective agents such as (S)-1-(2-ethylphenoxy)-3-((S)-1,2,3,4-tetrahydronaphthalen-1-ylamino)propan-2-ol oxalate (SR 59230A). In some embodiments, the pharmaceutical agent or the second pharmaceutical agent is a beta blocker selected from: alprenolol, bucindolol, carteolol, carvedilol, labetalol, nadolol, penbutolol, pindolol, propranolol, sotalol, timolol, acebutolol, atenolol, betaxolol, bisoprolol, celiprolol, esmolol, metoprolol, nebivolol, butaxamine, (2R,3R)-3-(isopropylamino)-1-(7-methyl-2,3-dihydro-1H-inden-4-yloxy)butan-2-ol (ICI-118,551), and (S)-1-(2-ethylphenoxy)-3-((S)-1,2,3,4-tetrahydronaphthalen-1-ylamino)propan-2-ol oxalate (SR 59230A).

Non-limiting examples of calcium channel blockers include dihydropyridine calcium channel blockers such as: amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, and pranidipine; phenylalkylamine calcium channel blockers such as: verapamil and gallopamil; benzothiazepine calcium channel blockers such as diltiazem; and non-selective calcium blockers such as mibefradil, bepridil, fluspirilene, and fendiline. In some embodiments, the pharmaceutical agent or the second pharmaceutical agent is a calcium channel blocker selected from: amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, verapamil, gallopamil, Diltiazem, mibefradil, bepridil, fluspirilene, and fendiline.

Non-limiting examples of diuretics include loop diuretics such as: furosemide, ethacrynic acid, torsemide and bumetanide; thiazide-type diuretics such as: hydrochlorothiazide; carbonic anhydrase inhibitors such as acetazolamide and methazolamide; potassium-sparing diuretics such as: spironolactone, potassium canreonate, amiloride and triamterene; calcium-sparing diuretics such as: the thiazides; osmotic diuretics such as: mannitol and glucose; and low ceiling diuretics such as: the thiazides; and digitalis. In some embodiments, the pharmaceutical agent or the second pharmaceutical agent is a diuretic selected from: furosemide, ethacrynic acid, torsemide, bumetanide, hydrochlorothiazide, acetazolamide, methazolamide, spironolactone, potassium canreonate, amiloride, triamterene; a thiazide, mannitol, glucose, and digitalis.

Non-limiting examples of nitrates include amyl nitrite, nitroglycerin, isosorbide dinitrate, isosorbide-5-mononitrate, and erythrityl tetranitrate. In some embodiments, the pharmaceutical agent or the second pharmaceutical agent is a nitrate selected from: amyl nitrite, nitroglycerin, isosorbide dinitrate, isosorbide-5-mononitrate, and erythrityl tetranitrate.

Non-limiting examples of statins include atorvastatin, cerivastatin, fluvastatin, lovastatin, mevastatin, pitavastatin, pravastatin, rosuvastatin, and simvastatin. In some embodiments, the pharmaceutical agent or the second pharmaceutical agent is a statin selected from: atorvastatin, cerivastatin, fluvastatin, lovastatin, mevastatin, pitavastatin, pravastatin, rosuvastatin, and simvastatin.

Non-limiting examples of anti-platelet agents include clopidogrel (Plavix®), prasugrel (Effient®), ticlopidine (Ticlid®), and temanogrel. In some embodiments, the pharmaceutical agent or the second pharmaceutical agent is an anti-platelet selected from: clopidogrel, prasugrel, ticlopidine, and temanogrel.

Non-limiting examples of endothelin receptor antagonists/inhibitors include bosentan, tezosentan, sitaxentan, ambrisentan, atrasentan, BQ-123 (i.e., cyclo(D-trp-D-asp-L-pro-D-val-L-leu)), and BQ-788 (i.e., N-cis-2,6-dimethylpiperidinocarbonyl-L-γ-MeLeu-D-Trp(MeOCO)-D-Nle-OH sodium salt).

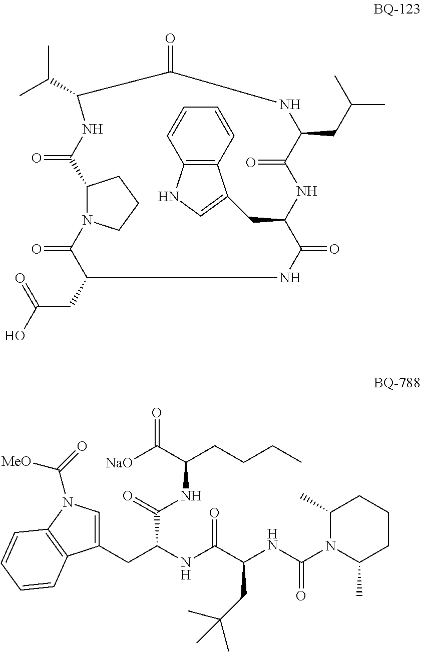

In some embodiments, the pharmaceutical agent or the second pharmaceutical agent is an endothelin receptor antagonist selected from: bosentan, tezosentan, sitaxentan, ambrisentan, atrasentan, BQ-123, and BQ-788.

Non-limiting examples of PDE5 inhibitors include sildenafil, avanafil, lodenafil, mirodenafil, sildenafil citrate, tadalafil, vardenafil, and udenafil. In some embodiments, the pharmaceutical agent or the second pharmaceutical agent is a PDE5 inhibitor selected from: sildenafil, avanafil, lodenafil, mirodenafil, sildenafil citrate, tadalafil, vardenafil, and udenafil.

Non-limiting examples of agents that inhibit the activity of TNF include small molecules, small interfering RNAs (siRNAs), anti-sense RNAs, antibodies that specifically bind to TNF, soluble TNF receptors, or dominant negative-TNF molecules (such as a dominant negative TNF protein or a nucleic acid encoding a dominant negative TNF protein). It is understood that an agent that inhibits TNF can be one that inhibits the ability of TNF to activate a receptor, but does not inhibit the binding of TNF to the receptor. Anti-TNF antibodies include, e.g., infliximab (Remicade®), D2E7 (adalumimab; Humira™), certolizumab (CDP-870), and CDP-571 (see, e.g., Sandborn et al., Gut 53(10):1485-1493 (2004); Choy et al., Rheumatology 41(10):1133-1137 (2002); and Kaushik et al., Expert Opinion on Biological Therapy 5(4):601-606(6) (2005)). Soluble TNF receptors include, e.g., etanercept (sTNF-RII:Fc; Enbrel®). Exemplary anti-TNF therapies are described in, e.g., U.S. Pat. No. 6,270,766.

Compounds of the present invention can also be used in combination with a cardioplegic solution. As used herein, a cardioplegic or cardioplesia solution is a solution infused into the heart, such as into the aortic root or the coronary ostia, to induce cardiac arrest during heart surgery or as a solution for use in the storage of the heart in preparation for transportation and eventual transplantation into the recipient. Compounds of the present invention can be used in combination with a variety of cardioplegic solutions known in the art. In some embodiments, the cardioplegic solution has a potassium chloride concentration in the range of about 15 mmol/L to about 35 mmol/L. In some embodiments, the cardioplegic solution has a potassium chloride concentration in the range of about 20 mmol/L to about 30 mmol/L. Examples of cardioplesia solutions include, but are not limited to, Plegisol™, Celsior®, Custodiol® HTK (Bretschneider's cardioplegic solution), CoStorSol® (University of Wisconsin) Solution, St. Thomas' Hospital solution (STH), and National Institutes of Health (NIH) solution.

One aspect of the present invention pertains to compounds of Formula (I) and compositions, pharmaceutical compositions, medicaments, unit dosage forms, methods, uses of compounds, compounds for use, and pharmaceutical products, each comprising a compound of Formula (I), in combination with one or more agents selected from the agents as described herein.

One embodiment pertains to methods for the treatment of a disorder, as described herein, in an individual comprising administering to an individual in need thereof, a therapeutically effective amount of: a compound of Formula (I), a composition comprising a compound of Formula (I), a pharmaceutical composition comprising a compound of Formula (I), a medicament comprising a compound of Formula (I), and/or a unit dosage form comprising a compound of Formula (I), in combination with a therapeutically effective amount of one or more agents selected from the agents as described herein.

One embodiment pertains to uses of: a compound of Formula (I), a composition comprising a compound of Formula (I), a pharmaceutical composition comprising a compound of Formula (I), a medicament comprising a compound of Formula (I), and/or a unit dosage form comprising a compound of Formula (I), in combination with one or more agents selected from the agents as described herein, in the manufacture of a medicament for the treatment of a disorder, as described herein, in an individual.

One embodiment pertains to: a compound of Formula (I), a composition comprising a compound of Formula (I), a pharmaceutical composition comprising a compound of Formula (I), a medicament comprising a compound of Formula (I), and/or a unit dosage form comprising a compound of Formula (I), in combination with one or more agents selected from the agents as described herein, for use in a method of treatment of the human or animal body by therapy.

One embodiment pertains to: a compound of Formula (I), a composition comprising a compound of Formula (I), a pharmaceutical composition comprising a compound of Formula (I), a medicament comprising a compound of Formula (I), and/or a unit dosage form comprising a compound of Formula (I), in combination with one or more agents selected from the agents as described herein for use, in a method of treatment of one or more disorder as described herein.

In some embodiments, a compound of Formula (I), either alone or present in a composition, a pharmaceutical composition, a medicament, and/or a unit dosage form; and the one or more agents are administered simultaneously, separately, or sequentially.

One aspect of the present invention relates to methods for preparing pharmaceutical products of the present invention comprising the steps: mixing said compound with a first pharmaceutically acceptable carrier to prepare a compound unit dosage form; mixing said second pharmaceutical agent with a second pharmaceutically acceptable carrier to prepare a second pharmaceutical agent unit dosage form; and combining said compound unit dosage form and said second pharmaceutical agent unit dosage form in a combined unit dosage form for simultaneous, separate, or sequential use.

In some embodiments, the first pharmaceutically acceptable carrier is different from the second pharmaceutically acceptable carrier. In some embodiments, the different pharmaceutically acceptable carriers are suitable for administration by the same route. In some embodiments, the different pharmaceutically acceptable carriers are suitable for administration by different routes. In some embodiments, the first pharmaceutically acceptable carrier is substantially the same as the second pharmaceutically acceptable carrier. In some embodiments, the substantially the same pharmaceutically acceptable carriers are suitable for oral administration.

### Certain Indications of the Present Invention:

One aspect of the present invention pertains to methods of the present invention; uses of the present invention; compounds of the present invention; crystalline forms of the present invention; compositions of the present invention; pharmaceutical products of the present invention; pharmaceutical compositions of the present invention; or pharmaceutical agents of the present invention; wherein the Mas receptor-mediated disorder is selected from: coronary heart disease, atherosclerosis, ischemia, reperfusion injury, reperfusion injury following cardioplegia, reperfusion injury following angioplasty, angina pectoris, myocardial infarction, no-reflow phenomenon, hypertension, pulmonary hypertension, anxiety, transient ischemic attack, erectile dysfunction, ischemic colitis, mesenteric ischemia, acute limb ischemia, skin discoloration caused by reduced blood flow to the skin, renal artery stenosis, renovascular hypertension, renal failure, chronic kidney disease, and diabetic nephropathy.

In some embodiments, the Mas receptor-mediated disorder is coronary heart disease. In some embodiments, the Mas receptor-mediated disorder is atherosclerosis. In some embodiments, the Mas receptor-mediated disorder is ischemia. In some embodiments, the Mas receptor-mediated disorder is reperfusion injury. In some embodiments, the Mas receptor-mediated disorder is reperfusion injury following cardioplegia. In some embodiments, the Mas receptor-mediated disorder is reperfusion injury following angioplasty. In some embodiments, the Mas receptor-mediated disorder is angina pectoris. In some embodiments, the Mas receptor-mediated disorder is myocardial infarction. In some embodiments, the Mas receptor-mediated disorder is the no-reflow phenomenon. In some embodiments, the Mas receptor-mediated disorder is hypertension. In some embodiments, the Mas receptor-mediated disorder is pulmonary hypertension. In some embodiments, the Mas receptor-mediated disorder is transient ischemic attack. In some embodiments, the Mas receptor-mediated disorder is erectile dysfunction. In some embodiments, the Mas receptor-mediated disorder is ischemic colitis. In some embodiments, the Mas receptor-mediated disorder is mesenteric ischemia. In some embodiments, the Mas receptor-mediated disorder is acute limb ischemia. In some embodiments, the Mas receptor-mediated disorder is skin discoloration caused by reduced blood flow to the skin. In some embodiments, the Mas receptor-mediated disorder is renal artery stenosis. In some embodiments, the Mas receptor-mediated disorder is renovascular hypertension. In some embodiments, the Mas receptor-mediated disorder is renal failure. In some embodiments, the Mas receptor-mediated disorder is chronic kidney disease. In some embodiments, the Mas receptor-mediated disorder is diabetic nephropathy.

One aspect of the present invention pertains to methods of the present invention; uses of the present invention; compounds of the present invention; crystalline forms of the present invention; compositions of the present invention; pharmaceutical products of the present invention; pharmaceutical compositions of the present invention; or pharmaceutical agents of the present invention; for the treatment or in a method of treatment of arrhythmia. In some embodiments, the arrhythmia is tachycardia. In some embodiments, the arrhythmia is bradycardia. In some embodiments, the arrhythmia is supraventricular arrhythmia. In some embodiments, the supraventricular arrhythmia is selected from: atrial fibrillation, atrial flutter, paroxysmal supraventricular tachycardia, and Wolff-Parkinson-White syndrome. In some embodiments, the arrhythmia is ventricular arrhythmia. In some embodiments, the ventricular arrhythmia is selected from: ventricular tachycardia and ventricular fibrillation. In some embodiments, the arrhythmia is reperfusion arrhythmia.

### Compositions and Formulations

One aspect of the present invention pertains to compositions comprising a compound of the present invention.

One aspect of the present invention pertains to compositions comprising a compound of the present invention and a pharmaceutically acceptable carrier.

One aspect of the present invention pertains to compositions obtained by a method of the present invention.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before lyophilization, or simply filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro et al.)

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

Pharmaceutical formulations include those suitable for oral, rectal, nasal, topical (including buccal and sub-lingual), vaginal or parenteral (including intramuscular, subcutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention and solvates, hydrates and other physiologically functional derivatives thereof can be used as active ingredients in pharmaceutical compositions, specifically as Mas receptor modulators. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds of the present invention can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis conducted or on whether further active compounds are administered in addition to the compounds of the present invention. Representative doses of the present invention include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt, solvate, or hydrate of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5% to about 90% of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” refers to the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Compounds of the present invention may be formulated as an aqueous solution, an aqua-alcoholic solution, a solid suspension, an emulsion, a liposomal suspension, or a freeze-dried powder for reconstitution. Such pharmaceutical compositions may be administered directly or as an admixture for further dilution/reconstitution. Route of administration includes intravenous bolus, intravenous infusion, irrigation, and instillation. Suitable solvents include water, alcohols, PEG, propylene glycol, and lipids; pH adjustments using an acid, e.g., HCl or citric acid, can be used to increase solubility and resulting compositions subjected to suitable sterilization procedures know in the art, such as, aseptic filtration. In some embodiments, the pH of the aqueous solution is about 2.0 to about 4.0. In some embodiments, the pH of the aqueous solution is about 2.5 to about 3.5.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well known to the person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

Compounds of the present invention may also be administered via a rapid dissolving or a slow release composition, wherein the composition includes a biodegradable rapid dissolving or slow release carrier (such as a polymer carrier and the like) and a compound of the invention.

Rapid dissolving or slow release carriers are well known in the art and are used to form complexes that capture therein an active compound(s) and either rapidly or slowly degrade/dissolve in a suitable environment (e.g., aqueous, acidic, basic, etc.). Such particles are useful because they degrade/dissolve in body fluids and release the active compound(s) therein. The particle size of a compound of the present invention, carrier or any excipient used in such a composition may be optimally adjusted using techniques known to those of ordinary skill in the art.

Particle size can play an important role in formulation. Reducing the size of the particles can be used to modify the physical characteristics. Particle size reduction increases both the number of particles and the amount of surface area per unit of volume. The increased surface area can improve the rate of solvation and therefore solubility. In addition, particle size reduction can improve gastrointestinal absorption for less soluble compounds. Particle size reduction can be obtained by any of the methods know in the art, for example, precipitation/crystallization, comminution (size reduction by a mechanical process), and the like, see for example Remington, The Science and Practice of Pharmacy, 20th Edition, 2000, Lippincott Williams & Wilkins, (Editors: Gennaro et al.).

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain compounds of the present invention which contain a carboxylic acid functional group may optionally exist as pharmaceutically acceptable salts containing non-toxic, pharmaceutically acceptable metal cations and cations derived from organic bases. Representative metals include, but are not limited to, aluminum, calcium, lithium, magnesium, potassium, sodium, zinc and the like. In some embodiments the pharmaceutically acceptable metal is sodium. Representative organic bases include, but are not limited to, benzathine (N¹,N²-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et al., Journal of Pharmaceutical Sciences, 66:1-19 (1977) and in “Handbook of Pharmaceutical Salts, Properties, Selection, and Use; Stahl, P. H. and Wermuth, C. G. (Eds.), Wiley-VCH (2002).

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

It is noted that when the Mas receptor modulators are utilized as active ingredients in pharmaceutical compositions, these are not intended for use in humans only, but in non-human mammals as well. Recent advances in the area of animal health-care mandate that consideration be given for the use of active agents, such as Mas receptor modulators, for the treatment of a Mas receptor-associated disease or disorder in companionship animals (e.g., cats, dogs, etc.) and in livestock animals (e.g., horses, cows, chickens, fish, etc.) Those of ordinary skill in the art are readily credited with understanding the utility of such compounds in such settings.

### Hydrates and Solvates

It is understood that when the phrase “pharmaceutically acceptable salts, solvates, and hydrates” or the phrase “pharmaceutically acceptable salt, solvate, or hydrate” is used when referring to compounds described herein, it embraces pharmaceutically acceptable solvates and/or hydrates of the compounds, pharmaceutically acceptable salts of the compounds, as well as pharmaceutically acceptable solvates and/or hydrates of pharmaceutically acceptable salts of the compounds. It is also understood that when the phrase “pharmaceutically acceptable solvates and hydrates” or the phrase “pharmaceutically acceptable solvate or hydrate” is used when referring to salts described herein, it embraces pharmaceutically acceptable solvates and/or hydrates of such salts.

It will be apparent to those skilled in the art that the dosage forms described herein may comprise, as the active component, either a compound described herein or a pharmaceutically acceptable salt or as a pharmaceutically acceptable solvate or hydrate thereof. Moreover, various hydrates and solvates of the compounds described herein and their salts can find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present invention pertains to methods of administering hydrates and solvates of compounds described herein and/or their pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

One aspect of the present invention pertains to solvates of salts of compounds of the present invention. One aspect of the present invention pertains to solvates of a hydrochloride salt of a compound of the present invention. In some embodiments the salt is (S)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide.

### Polymorphs and Pseudopolymorphs

Polymorphism is the ability of a substance to exist as two or more crystalline phases that have different arrangements and/or conformations of the molecules in the crystal lattice. Compounds that form polymorphs show the same properties in the liquid or gaseous state, but in the solid state their polymorphs behave differently.

Besides single-component polymorphs, drugs can also exist as salts and other multicomponent crystalline phases. For example, solvates and hydrates may contain an API host and either solvent or water molecules, respectively, as guests. Analogously, when the guest compound is a solid at room temperature, the resulting form is often called a cocrystal. Salts, solvates, hydrates, and cocrystals may show polymorphism as well. Crystalline phases that share the same API host, but differ with respect to their guests, may be referred to as pseudopolymorphs of one another.

Solvates contain molecules of the solvent of crystallization in a definite crystal lattice. Solvates, in which the solvent of crystallization is water, are termed hydrates. Because water is a constituent of the atmosphere, hydrates of drugs may be formed rather easily and may be thermodynamically favored over anhydrous polymorphs.

By way of example, Stahly recently published a polymorph screen of 245 compounds consisting of a “wide variety of structural types” that revealed about 90% of the compounds exhibited multiple solid forms. Overall, approximately half the compounds were polymorphic, often having one to three forms. About one-third of the compounds formed hydrates, and about one-third formed other solvates. Data from cocrystal screens of 64 compounds showed that 60% formed cocrystals other than hydrates or solvates (G. P. Stahly, Crystal Growth & Design (2007), 7(6), 1007-1026).

Crystalline forms, such as those described herein, can be identified by their unique solid state signature with respect to, for example, differential scanning calorimetry (DSC), powder X-ray diffraction (PXRD), and other solid state methods.

Further characterization with respect to water or solvent content of crystalline forms can be determined by any of the following methods, for example, thermogravimetric analysis (TGA), Karl Fischer analysis, and the like.

For DSC, it is known that the temperatures observed will depend upon sample purity, the rate of temperature change, as well as sample preparation technique and the particular instrument employed. Thus, the values reported herein relating to DSC thermograms can vary by plus or minus about 4° C. (±4° C.). The values reported herein relating to DSC thermograms can also vary by plus or minus about 20 joules per gram (±20 joules per gram).

In some embodiments, the DSC thermogram values reported herein relate to desolvation events. When DSC thermogram values reported herein relate to desolvation events, the values reported herein are estimates. Scan rate and pan closure can influence DSC values for desolvation events, which can vary by plus or minus about 25° C. DSC values for desolvation events reported herein were recorded using a sample in an aluminum pan with an uncrimped lid and a scan rate of 10° C./min.

For PXRD, the relative intensities of the peaks can vary, depending upon the sample preparation technique, the sample mounting procedure and the particular instrument employed. Moreover, instrument variation and other factors can often affect the 2θ values. Therefore, the peak assignments of diffraction patterns can vary by plus or minus 0.2 °2θ(±0.2 °2θ).

For TGA, the features reported herein can vary by plus or minus about 5° C. (±5° C.). The TGA features reported herein can also vary by plus or minus about 2% (±2%) weight change due to, for example, sample variation.

Further characterization with respect to hygroscopicity of the crystalline forms can be gauged by, for example, dynamic moisture sorption (DMS). The DMS features reported herein can vary by plus or minus about 5% (±5%) relative humidity. The DMS features reported herein can also vary by plus or minus about 5% (±5%) weight change.

One aspect of the present invention is directed, inter alia, to crystalline forms of (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) and salts, solvates and hydrates thereof.

### Examples of Pharmaceutically Acceptable Salts, Solvates, Hydrates, and Crystalline Forms of the Present Invention

One aspect of the present invention is directed to compounds selected from compounds of Formula (I) and pharmaceutically acceptable salts, solvates and hydrates thereof.

One aspect of the present invention is directed to (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) and pharmaceutically acceptable salts (such as, HCl, sulfate, and mesylate salts), solvates and hydrates thereof.

One aspect of the present invention is directed to (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide.

One aspect of the present invention relates a crystalline form of (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide.

One aspect of the present invention is directed to (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride salt and solvates and hydrates thereof. One aspect of the present invention is directed to a crystalline form of anhydrous (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride. One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride hydrate. One aspect of the present invention is directed to a crystalline form of (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride solvate.

One aspect of the present invention is directed to (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide sulfate salt and solvates and hydrates thereof. One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide sulfate solvate.

One aspect of the present invention is directed to (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide di-mesylate. One aspect of the present invention is directed to a crystalline form of anhydrous (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide di-mesylate.

### Free Base and Crystalline Forms

One aspect of the present invention relates crystalline forms of (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170).

One crystalline form of Compound 170 was prepared according to Example 2.5A. The solid-state properties for a crystalline form as determined by PXRD and DSC are summarized in Table 1A below.

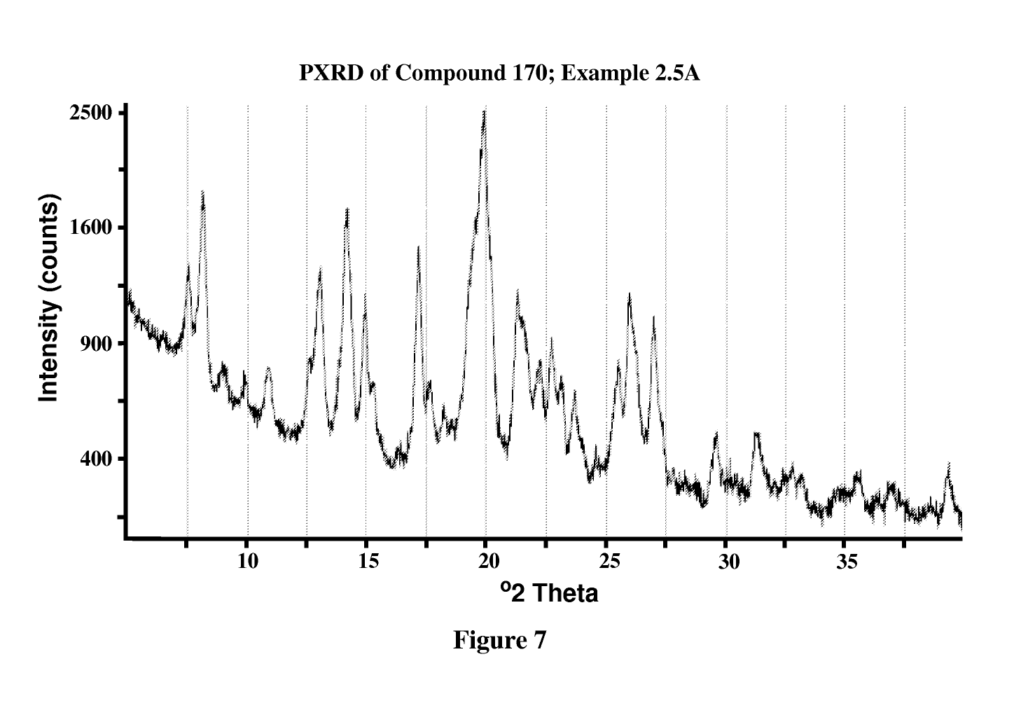

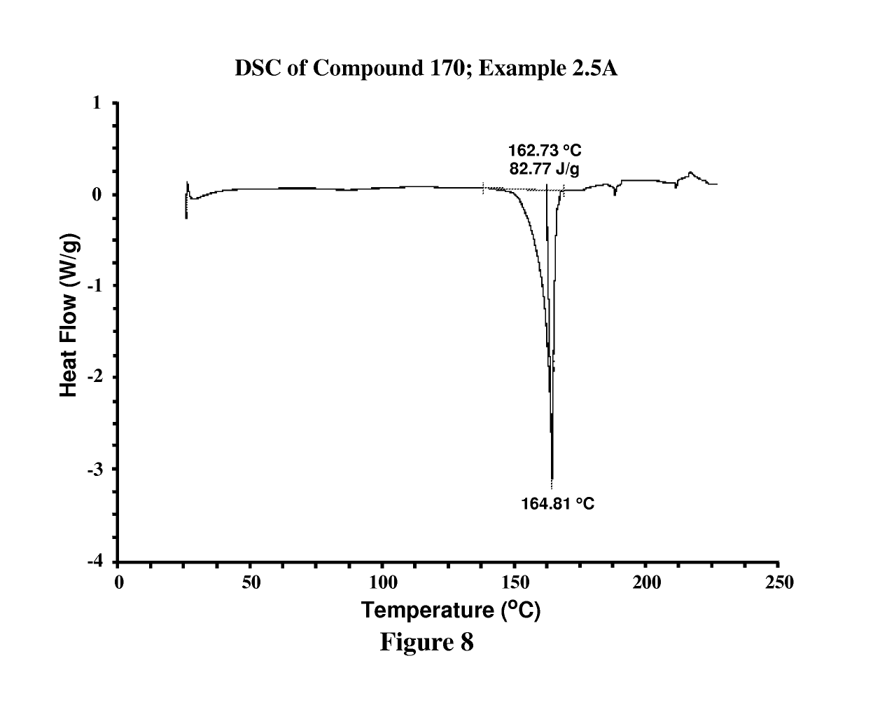

TABLE 1A

| | Compound 170 (Example 2.5A) |
|---|---|
| PXRD | FIG. 7: Peaks of about ≧25% relative intensity at 7.56, 8.15, |
| | 13.05, 14.17, 14.95, 17.17, 19.90, 21.31, 22.73, 25.52, 25.98, |
| | and 27.01 °2θ |
| DSC | FIG. 8: Endotherm extrapolated onset temperature: about |
| | 164° C. |

Certain powder X-ray diffraction peaks for (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) as prepared according to Example 2.5A are shown in Table 2A below.

TABLE 2A

| | d-spacing | Rel. Int. |
|---|---|---|
| Pos. [°2θ.] | [Å] | [%] |
| 5.29 | 16.72 | 3.07 |
| 7.56 | 11.70 | 26.29 |
| 8.15 | 10.85 | 53.56 |
| 9.00 | 9.83 | 6.18 |
| 9.92 | 8.91 | 6.70 |
| 10.91 | 8.11 | 12.67 |
| 12.61 | 7.02 | 17.98 |
| 13.05 | 6.79 | 41.36 |
| 14.17 | 6.25 | 62.33 |
| 14.95 | 5.92 | 33.85 |
| 15.27 | 5.80 | 15.93 |
| 17.17 | 5.16 | 51.91 |
| 17.64 | 5.03 | 16.42 |
| 18.24 | 4.86 | 10.35 |
| 19.24 | 4.61 | 36.58 |
| 19.54 | 4.54 | 59.66 |
| 19.90 | 4.46 | 100.00 |
| 20.24 | 4.39 | 42.40 |
| 21.31 | 4.17 | 39.16 |
| 21.68 | 4.10 | 25.88 |
| 22.22 | 4.00 | 21.32 |
| 22.73 | 3.91 | 27.48 |
| 23.13 | 3.84 | 17.67 |
| 23.71 | 3.75 | 16.73 |
| 25.52 | 3.49 | 24.16 |
| 25.98 | 3.43 | 41.67 |
| 26.28 | 3.39 | 26.90 |
| 27.01 | 3.30 | 33.69 |
| 29.60 | 3.02 | 11.70 |
| 31.29 | 2.86 | 12.40 |
| 32.66 | 2.74 | 5.78 |
| 33.15 | 2.70 | 5.72 |
| 35.53 | 2.53 | 5.90 |
| 36.91 | 2.44 | 4.63 |
| 39.30 | 2.29 | 7.20 |

One aspect of the present invention is directed to a crystalline form of Compound 170 (as prepared according to Example 2.5A), having a powder X-ray diffraction pattern comprising a peak, in terms of 2θ, at 19.90°±0.20°. One aspect of the present invention is directed to a crystalline form of Compound 170, having a powder X-ray diffraction pattern comprising a peak, in terms of 2θ, at 17.17°±0.20°. One aspect of the present invention is directed to a crystalline form of Compound 170, having a powder X-ray diffraction pattern comprising a peak, in terms of 2θ, at 14.17°±0.20°. One aspect of the present invention is directed to a crystalline form of Compound 170, having a powder X-ray diffraction pattern comprising peaks, in terms of 2θ, at 14.17°±0.20°, 17.17°±0.20°, and 19.90°±0.20°. One aspect of the present invention is directed to a crystalline form of Compound 170, having a powder X-ray diffraction pattern comprising peaks, in terms of 2θ, at 8.15°±0.20°, 14.17°±0.20°, 17.17°±0.20°, 19.90°±0.20°, and 25.98°±0.20°. One aspect of the present invention is directed to a crystalline form of Compound 170, having a powder X-ray diffraction pattern comprising peaks, in terms of 2θ, at 8.15°±0.20°, 13.05°±0.20°, 14.17°±0.20°, 17.17°±0.20°, 19.90°±0.20°, 21.31°±0.20°, and 25.98°±0.20°. One aspect of the present invention is directed to a crystalline form of Compound 170, having a powder X-ray diffraction pattern comprising peaks, in terms of 2θ, at 8.15°±0.20°, 13.05°±0.20°, 14.17°±0.20°, 14.95°±0.20°, 17.17°±0.20°, 19.90°±0.20°, 21.31°±0.20°, 25.98°±0.20°, and 27.01°±0.20°. One aspect of the present invention is directed to a crystalline form of Compound 170, having a powder X-ray diffraction pattern comprising peaks, in terms of 2θ, at 7.56°±0.20°, 8.15°±0.20°, 13.05°±0.20°, 14.17°±0.20°, 14.95°±0.20°, 17.17°±0.20°, 19.90°±0.20°, 21.31°±0.20°, 22.73°±0.20°, 25.52°±0.20°, 25.98°±0.20°, and 27.01°±0.20°. One aspect of the present invention is directed to a crystalline form of Compound 170, having a powder X-ray diffraction pattern comprising one or more peaks listed in Table 2A. One aspect of the present invention is directed to a crystalline form of Compound 170, having a powder X-ray diffraction pattern substantially as shown in FIG. 7, wherein by “substantially” is meant that the reported peaks can vary by about ±0.2 °2θ and also that the relative intensities of the reported peaks can vary. One aspect of the present invention is directed to a crystalline form of Compound 170, having a differential scanning calorimetry thermogram comprising an endotherm with an extrapolated onset temperature between 158° C.±4° C. and 168° C.±4° C. One aspect of the present invention is directed to a crystalline form of Compound 170, having a differential scanning calorimetry thermogram comprising an endotherm with an extrapolated onset temperature at 164° C.±4° C. One aspect of the present invention is directed to a crystalline form of Compound 170, having a differential scanning calorimetry thermogram comprising an endotherm with an associated heat flow of 83 joules per gram±20 joules per gram. One aspect of the present invention is directed to a crystalline form of Compound 170, having a differential scanning calorimetry thermogram substantially as shown in FIG. 8, wherein by “substantially” is meant that the reported DSC features can vary by about ±4° C. and by about ±20 joules per gram.

Another crystalline form of Compound 170 was prepared according to Example 2.5B. The solid-state properties for the crystalline form as determined by PXRD, DSC, and DMS are summarized in Table 1B below.

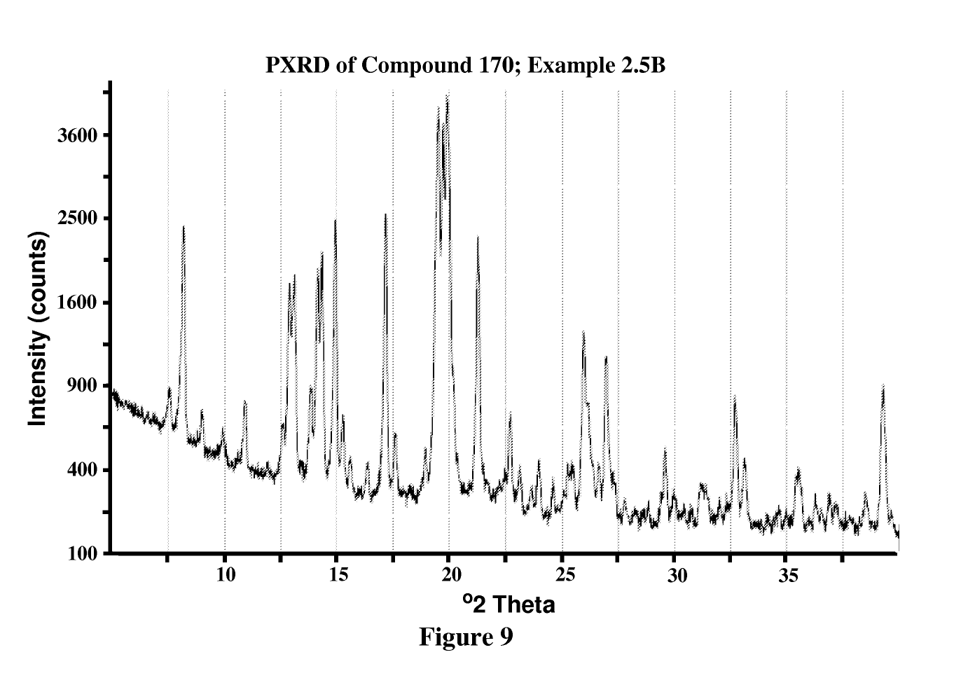

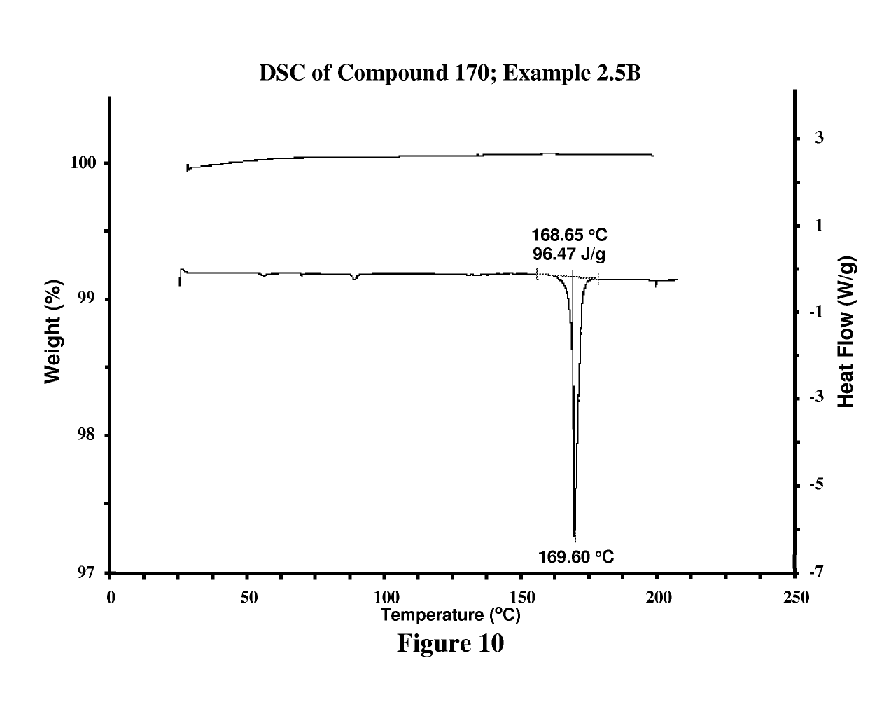

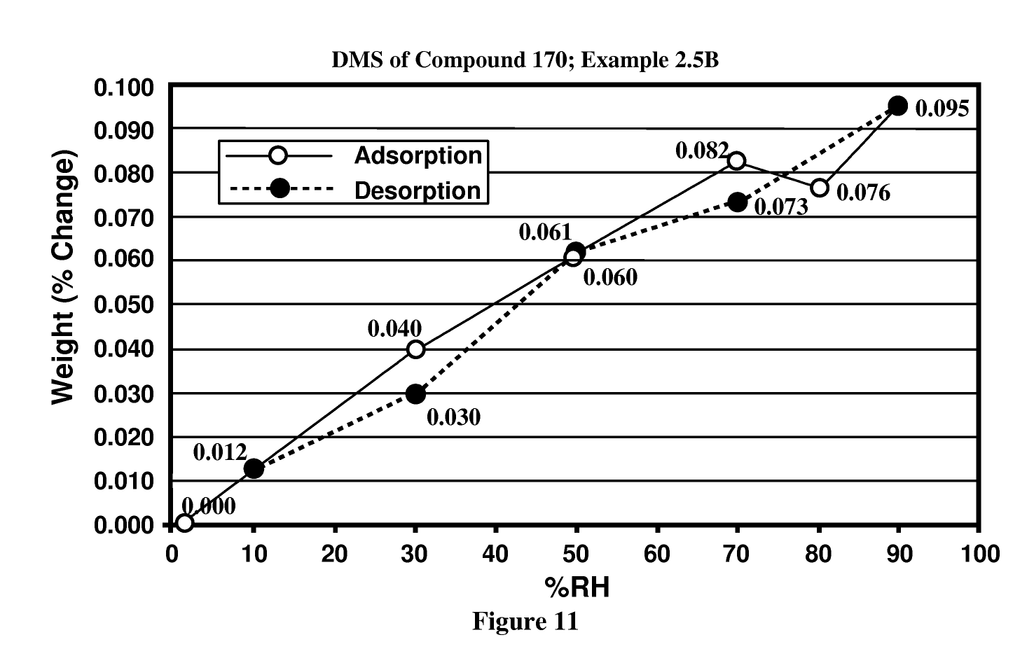

TABLE 1B

| | Compound 170 (Example 2.5B) |
|---|---|
| PXRD | FIG. 9: Peaks of about ≧17% relative intensity at 8.18, 12.89, |
| | 13.11, 14.33, 14.93, 17.16, 19.51, 19.72, 19.91, 21.28, 25.98, |
| | 26.98, 32.71, and 39.28 °2θ |
| DSC | FIG. 10: Endotherm extrapolated onset temperature: about |
| | 169° C. |
| DMS | FIG. 11: This polymorph of the free base is non-hygroscopic, |
| | increasing ~0.1% weight at 90% RH and 25° C. |

Certain powder X-ray diffraction peaks for (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) as prepared according to Example 2.5B are shown in Table 2B below.

TABLE 2B

| | d-spacing | Rel. Int. |
|---|---|---|
| Pos. [°2θ.] | [Å] | [%] |
| 7.57 | 11.67 | 7.69 |
| 8.18 | 10.80 | 49.33 |
| 9.01 | 9.82 | 6.81 |
| 10.92 | 8.11 | 10.93 |
| 12.60 | 7.02 | 7.85 |
| 12.89 | 6.87 | 39.02 |
| 13.11 | 6.75 | 40.63 |
| 13.84 | 6.40 | 14.73 |
| 14.15 | 6.26 | 42.73 |
| 14.33 | 6.18 | 48.20 |
| 14.93 | 5.93 | 59.05 |
| 15.27 | 5.80 | 11.48 |
| 17.16 | 5.17 | 60.59 |
| 17.60 | 5.04 | 8.77 |
| 18.94 | 4.69 | 6.21 |
| 19.51 | 4.55 | 97.59 |
| 19.72 | 4.50 | 93.09 |
| 19.91 | 4.46 | 100.00 |
| 21.28 | 4.18 | 53.08 |
| 22.71 | 3.92 | 13.28 |
| 23.13 | 3.84 | 5.19 |
| 23.98 | 3.71 | 6.61 |
| 25.29 | 3.52 | 5.48 |
| 25.47 | 3.50 | 5.84 |
| 25.98 | 3.43 | 30.65 |
| 26.16 | 3.41 | 15.81 |
| 26.64 | 3.35 | 6.33 |
| 26.98 | 3.30 | 25.40 |
| 29.59 | 3.02 | 8.83 |
| 32.71 | 2.74 | 17.88 |
| 33.13 | 2.70 | 7.74 |
| 35.43 | 2.53 | 5.49 |
| 35.59 | 2.52 | 5.26 |
| 39.28 | 2.29 | 19.81 |

One aspect of the present invention is directed to a crystalline form of Compound 170 (as prepared according to Example 2.5B), having a powder X-ray diffraction pattern comprising a peak, in terms of 2θ, at 19.91°±0.20°. One aspect of the present invention is directed to a crystalline form of Compound 170, having a powder X-ray diffraction pattern comprising a peak, in terms of 2θ, at 21.28°±0.20°. One aspect of the present invention is directed to a crystalline form of Compound 170, having a powder X-ray diffraction pattern comprising a peak, in terms of 2θ, at 17.16°±0.20°. One aspect of the present invention is directed to a crystalline form of Compound 170, having a powder X-ray diffraction pattern comprising peaks, in terms of 2θ, at 17.16°±0.20°, 19.91°±0.20°, and 21.28°±0.20°. One aspect of the present invention is directed to a crystalline form of Compound 170, having a powder X-ray diffraction pattern comprising peaks, in terms of 2θ, at 14.93°±0.20°, 17.16°±0.20°, 19.51°±0.20°, 19.72°±0.20°, 19.91°±0.20°, and 21.28°±0.20°. One aspect of the present invention is directed to a crystalline form of Compound 170, having a powder X-ray diffraction pattern comprising peaks, in terms of 2θ, at 8.18°±0.20°, 13.11°±0.20°, 14.33°±0.20°, 14.93°±0.20°, 17.16°±0.20°, 19.51°±0.20°, 19.72°±0.20°, 19.91°±0.20°, and 21.28°±0.20°. One aspect of the present invention is directed to a crystalline form of Compound 170, having a powder X-ray diffraction pattern comprising peaks, in terms of 2θ, at 8.18°±0.20°, 12.89°±0.20°, 13.11°±0.20°, 14.33°±0.20°, 14.93°±0.20°, 17.16°±0.20°, 19.51°±0.20°, 19.72°±0.20°, 19.91°±0.20°, 21.28°±0.20°, 25.98°±0.20°, and 26.98°±0.20°. One aspect of the present invention is directed to a crystalline form of Compound 170, having a powder X-ray diffraction pattern comprising peaks, in terms of 2θ, at 8.18°±0.20°, 12.89°±0.20°, 13.11°±0.20°, 14.33°±0.20°, 14.93°±0.20°, 17.16°±0.20°, 19.51°±0.20°, 19.72°±0.20°, 19.91°±0.20°, 21.28°±0.20°, 25.98°±0.20°, 26.98°±0.20°, 32.71°±0.20°, and 39.28°±0.20°. One aspect of the present invention is directed to a crystalline form of Compound 170, having a powder X-ray diffraction pattern comprising one or more peaks listed in Table 2B. One aspect of the present invention is directed to a crystalline form of Compound 170, having a powder X-ray diffraction pattern substantially as shown in FIG. 9, wherein by “substantially” is meant that the reported peaks can vary by about ±0.2 °2θ, and also that the relative intensities of the reported peaks can vary. One aspect of the present invention is directed to a crystalline form of Compound 170, having a differential scanning calorimetry thermogram comprising an endotherm with an extrapolated onset temperature between 163° C.±4° C. and 173° C.±4° C. One aspect of the present invention is directed to a crystalline form of Compound 170, having a differential scanning calorimetry thermogram comprising an endotherm with an extrapolated onset temperature at 169° C.±4° C. One aspect of the present invention is directed to a crystalline form of Compound 170, having a differential scanning calorimetry thermogram comprising an endotherm with an associated heat flow of 96 joules per gram±20 joules per gram. One aspect of the present invention is directed to a crystalline form of Compound 170, having a differential scanning calorimetry thermogram substantially as shown in FIG. 10, wherein by “substantially” is meant that the reported DSC features can vary by about ±4° C. and by about ±20 joules per gram. One aspect of the present invention is directed to a crystalline form of Compound 170, having a dynamic moisture sorption profile substantially as shown in FIG. 11, wherein by “substantially” is meant that the reported DMS features can vary by about ±5% relative humidity and by about ±5% weight change.

### Dihydrochloride (i.e., Di-HCl) Salt and Crystalline Forms

One aspect of the present invention is directed to (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) dihydrochloride and solvates and hydrates thereof.

a. Dihydrochloride Form

One aspect of the present invention is directed to the anhydrous form of (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride. Certain solid-state properties of the crystalline form of the anhydrous dihydrochloride are summarized in Table 3 below.

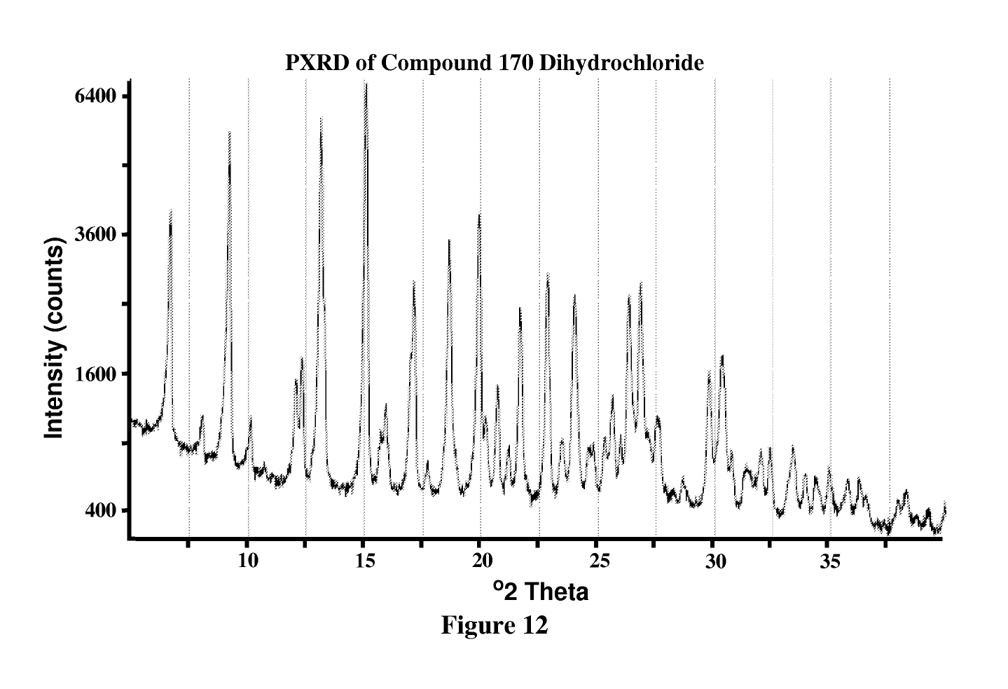

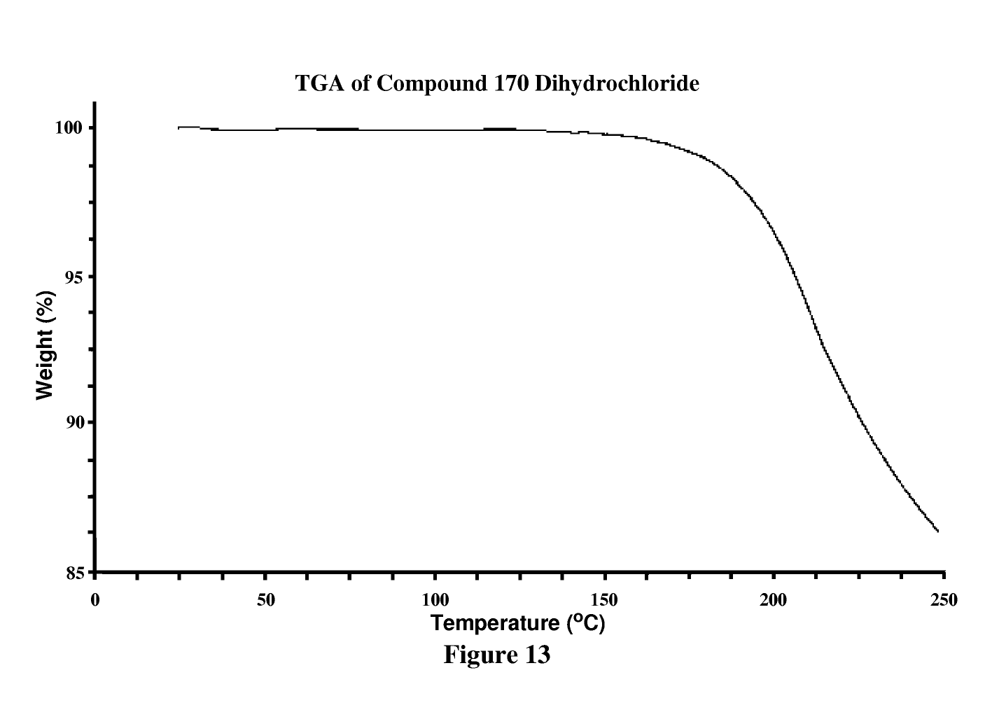

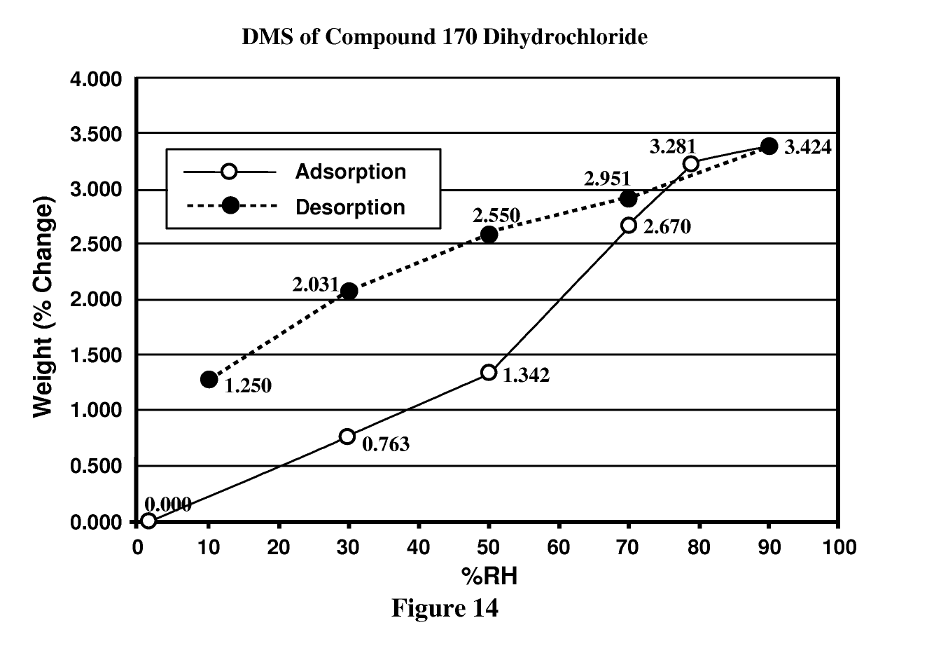

TABLE 3

| | Compound 170 dihydrochloride |
|---|---|
| PXRD | FIG. 12: Peaks of about ≧20% relative intensity at 6.69, 9.22, |
| | 12.33, 13.14, 15.08, 17.13, 18.64, 19.92, 21.69, 22.85, 24.01, |
| | 26.34, 26.84, 29.78, and 30.33 °2θ |
| TGA | FIG. 13: No substantial weight loss out to about 150° C. |
| DSC | There is no definitive melting using a DSC run at 10° C./min. |
| DMS | FIG. 14: The total weight gain at 90% RH and 25° C., after a |
| | drying period, was about 3.4%. It was shown by PXRD of the |
| | sample post-DMS analysis that it had converted to the known |
| | small-channel hydrate form (see Table 7). |

Certain powder X-ray diffraction peaks for the anhydrous form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride are shown in Table 4 below.

TABLE 4

| | d-spacing | Rel. Int. |
|---|---|---|
| Pos. [°2θ.] | [Å] | [%] |
| 6.69 | 13.22 | 50.60 |
| 8.04 | 11.00 | 6.65 |
| 9.22 | 9.60 | 79.77 |
| 10.11 | 8.75 | 7.18 |
| 10.72 | 8.26 | 1.94 |
| 12.07 | 7.33 | 15.18 |
| 12.33 | 7.18 | 20.00 |
| 13.14 | 6.74 | 84.91 |
| 15.08 | 5.88 | 100.00 |
| 15.68 | 5.65 | 7.67 |
| 15.91 | 5.57 | 12.02 |
| 17.13 | 5.18 | 37.34 |
| 17.70 | 5.01 | 4.05 |
| 18.64 | 4.76 | 49.18 |
| 19.92 | 4.46 | 56.58 |
| 20.20 | 4.40 | 10.75 |
| 20.71 | 4.29 | 15.81 |
| 21.18 | 4.19 | 5.76 |
| 21.69 | 4.10 | 31.04 |
| 22.85 | 3.89 | 39.74 |
| 23.49 | 3.79 | 7.71 |
| 24.01 | 3.71 | 35.02 |
| 24.63 | 3.61 | 6.10 |
| 24.80 | 3.59 | 6.91 |
| 25.30 | 3.52 | 7.99 |
| 25.63 | 3.48 | 14.81 |
| 25.98 | 3.43 | 7.61 |
| 26.34 | 3.38 | 34.78 |
| 26.84 | 3.32 | 38.36 |
| 27.54 | 3.24 | 10.62 |
| 27.64 | 3.23 | 10.48 |
| 28.66 | 3.11 | 3.10 |
| 29.78 | 3.00 | 19.27 |
| 30.33 | 2.95 | 22.04 |
| 30.75 | 2.91 | 6.48 |
| 31.27 | 2.86 | 3.84 |
| 32.00 | 2.80 | 6.78 |
| 32.39 | 2.76 | 7.62 |
| 33.37 | 2.68 | 7.98 |
| 33.90 | 2.64 | 4.73 |
| 34.35 | 2.61 | 4.25 |
| 34.93 | 2.57 | 5.46 |
| 35.73 | 2.51 | 4.53 |
| 36.22 | 2.48 | 4.64 |
| 36.48 | 2.46 | 3.01 |
| 37.88 | 2.38 | 2.85 |
| 38.23 | 2.35 | 3.77 |
| 39.16 | 2.30 | 1.71 |

One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) dihydrochloride.

One aspect of the present invention is directed to a crystalline form of Compound 170 dihydrochloride, having a powder X-ray diffraction pattern comprising a peak, in terms of 2θ, at 15.08°±0.20°. One aspect of the present invention is directed to a crystalline form of Compound 170 dihydrochloride, having a powder X-ray diffraction pattern comprising a peak, in terms of 2θ, at 13.14°±0.20°. One aspect of the present invention is directed to a crystalline form of Compound 170 dihydrochloride, having a powder X-ray diffraction pattern comprising a peak, in terms of 2θ, at 9.22°±0.20°. One aspect of the present invention is directed to a crystalline form of Compound 170 dihydrochloride, having a powder X-ray diffraction pattern comprising peaks, in terms of 2θ, at 9.22°±0.20°, 13.14°±0.20°, and 15.08°±0.20°. One aspect of the present invention is directed to a crystalline form of Compound 170 dihydrochloride, having a powder X-ray diffraction pattern comprising peaks, in terms of 2θ, at 6.69°±0.20°, 9.22°±0.20°, 13.14°±0.20°, 15.08°±0.20°, and 18.64°±0.20°. One aspect of the present invention is directed to a crystalline form of Compound 170 dihydrochloride, having a powder X-ray diffraction pattern comprising peaks, in terms of 2θ, at 6.69°±0.20°, 9.22°±0.20°, 13.14°±0.20°, 15.08°±0.20°, 17.13°±0.20°, and 18.64°±0.20°. One aspect of the present invention is directed to a crystalline form of Compound 170 dihydrochloride, having a powder X-ray diffraction pattern comprising peaks, in terms of 2θ, at 6.69°±0.20°, 9.22°±0.20°, 13.14°±0.20°, 15.08°±0.20°, 17.13°±0.20°, 18.64°±0.20°, 19.92°±0.20°, 22.85°±0.20°, and 24.01°±0.20°. One aspect of the present invention is directed to a crystalline form of Compound 170 dihydrochloride, having a powder X-ray diffraction pattern comprising peaks, in terms of 2θ, at 6.69°±0.20°, 9.22°±0.20°, 12.33°±0.20°, 13.14°±0.20°, 15.08°±0.20°, 17.13°±0.20°, 18.64°±0.20°, 19.92°±0.20°, 22.85°±0.20°, 24.01°±0.20°, 26.34°±0.20°, and 26.84°±0.20°. One aspect of the present invention is directed to a crystalline form of Compound 170 dihydrochloride, having a powder X-ray diffraction pattern comprising one or more peaks listed in Table 4. One aspect of the present invention is directed to a crystalline form of Compound 170 dihydrochloride, having a powder X-ray diffraction pattern substantially as shown in FIG. 12, wherein by “substantially” is meant that the reported peaks can vary by about ±0.2 °2θ and also that the relative intensities of the reported peaks can vary. One aspect of the present invention is directed to a crystalline form of Compound 170 dihydrochloride, having a thermogravimetric analysis profile substantially as shown in FIG. 13, wherein by “substantially” is meant that the reported TGA features can vary by about ±5° C. and by about ±2% weight change. One aspect of the present invention is directed to a crystalline form of Compound 170 dihydrochloride, having a dynamic moisture sorption profile substantially as shown in FIG. 14, wherein by “substantially” is meant that the reported DMS features can vary by about ±5% relative humidity and by about ±5% weight change.

B. Dihydrochloride Hydrate

One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) dihydrochloride hydrate. Certain solid-state properties of the crystalline form of the dihydrochloride hydrate are summarized in Table 5 below.

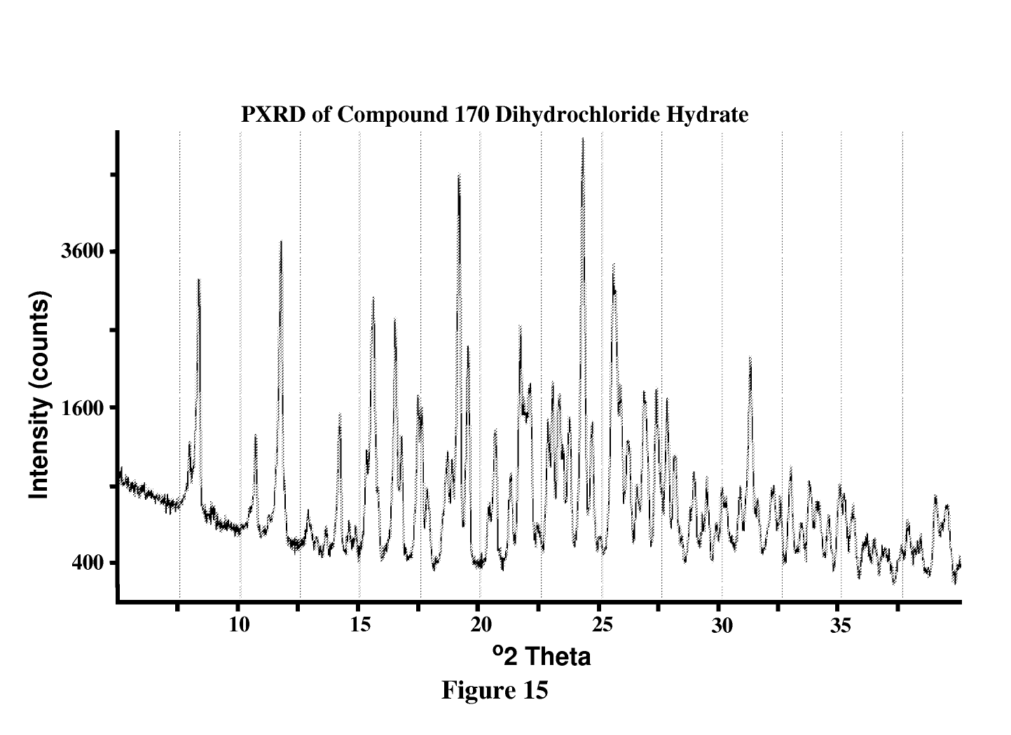

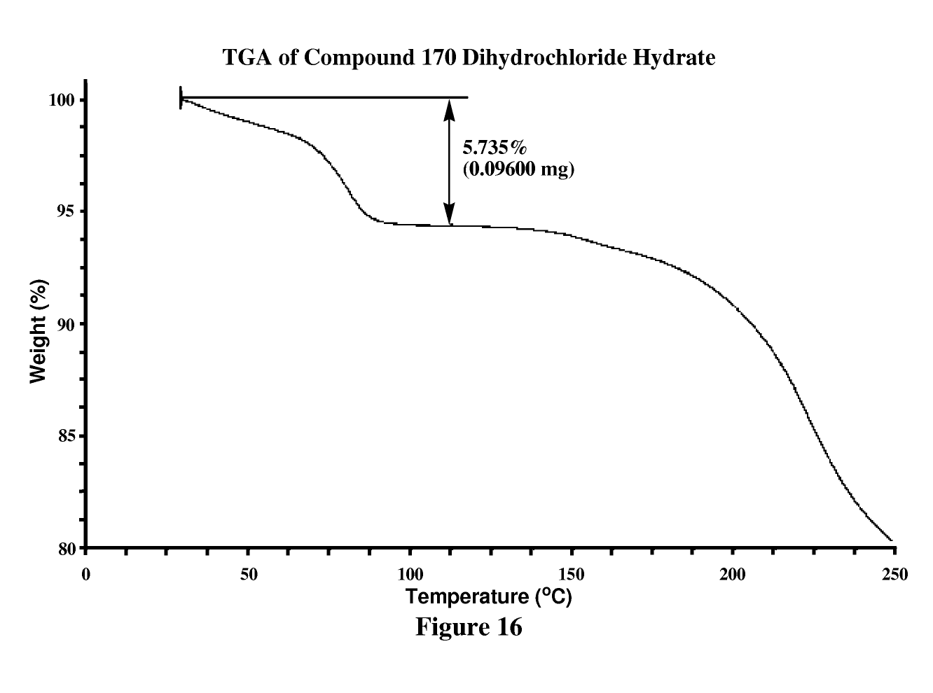

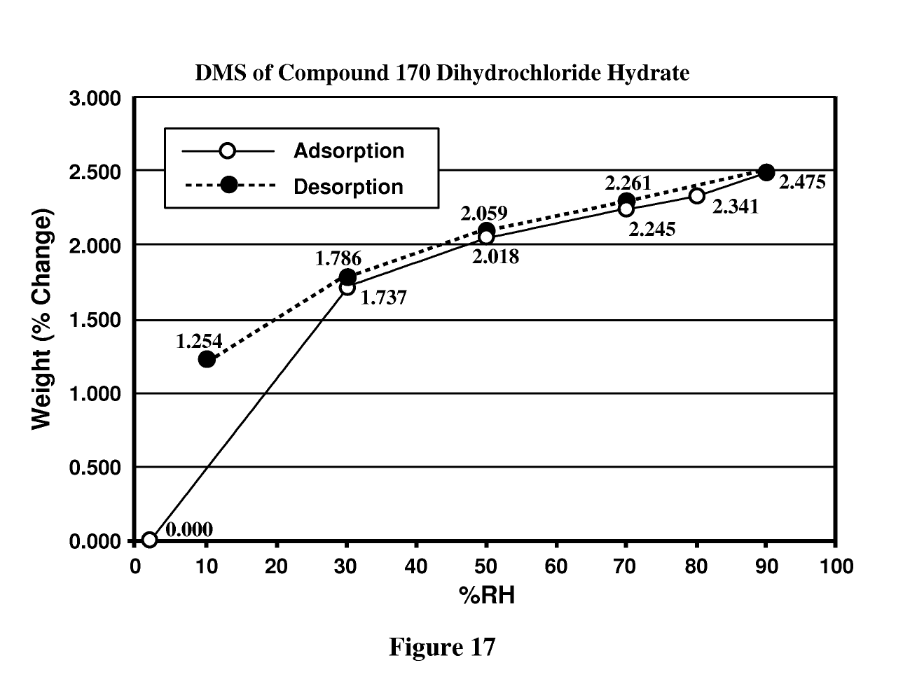

TABLE 5

| | Compound 170 dihydrochloride hydrate |
|---|---|
| PXRD | FIG. 15: Peaks of about ≧20% relative intensity at 8.30, 11.71, |
| | 15.54, 16.45, 17.40, 19.10, 19.47, 21.64, 21.79, 22.04, 22.98, |
| | 23.25, 23.67, 24.23, 25.51, 26.78, 27.29, 27.74, and 31.18 °2θ |
| TGA | FIG. 16: Decrease in weight of about 5.7% out to about 125° C. |
| | The amount of water loss is not stoichiometric and may be |
| | characterized as a channel hydrate or a combination of a channel |
| | and lattice-site hydrate. |
| DMS | FIG. 17: The sample lost 1.8% weight during a drying step (not |
| | shown in this isotherm plot). This more loosely held water likely |
| | corresponds to channel hydration. The total weight gain from the |
| | dried state was about 2.5%. The sample picked up only about |
| | 0.7% water from 30% RH to 90% RH at 25° C. |

Certain powder X-ray diffraction peaks for (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) dihydrochloride hydrate are shown in Table 6 below.

TABLE 6

| | d-spacing | Rel. Int. |
|---|---|---|
| Pos. [°2θ.] | [Å] | [%] |
| 7.91 | 11.18 | 10.62 |
| 8.30 | 10.66 | 48.67 |
| 10.66 | 8.30 | 15.05 |
| 11.71 | 7.56 | 63.48 |
| 14.14 | 6.26 | 19.60 |
| 15.27 | 5.80 | 15.30 |
| 15.54 | 5.70 | 48.59 |
| 16.45 | 5.39 | 42.99 |
| 16.71 | 5.31 | 17.83 |
| 17.40 | 5.10 | 25.36 |
| 17.54 | 5.06 | 23.55 |
| 18.62 | 4.77 | 15.13 |
| 18.80 | 4.72 | 14.41 |
| 19.10 | 4.65 | 86.82 |
| 19.47 | 4.56 | 36.52 |
| 20.60 | 4.31 | 19.29 |
| 21.24 | 4.18 | 11.45 |
| 21.64 | 4.11 | 41.62 |
| 21.79 | 4.08 | 35.68 |
| 22.04 | 4.03 | 28.25 |
| 22.79 | 3.90 | 19.66 |
| 22.98 | 3.87 | 28.62 |
| 23.25 | 3.83 | 25.48 |
| 23.42 | 3.80 | 15.67 |
| 23.67 | 3.76 | 20.59 |
| 24.23 | 3.67 | 100.00 |
| 24.61 | 3.62 | 19.98 |
| 25.51 | 3.49 | 57.94 |
| 25.60 | 3.49 | 49.82 |
| 25.80 | 3.45 | 28.17 |
| 26.11 | 3.41 | 16.91 |
| 26.78 | 3.33 | 26.89 |
| 26.91 | 3.32 | 16.22 |
| 27.29 | 3.26 | 27.34 |
| 27.74 | 3.21 | 25.39 |
| 28.06 | 3.18 | 14.34 |
| 28.86 | 3.09 | 11.88 |
| 29.38 | 3.04 | 11.00 |
| 30.78 | 2.90 | 10.08 |
| 31.18 | 2.87 | 33.37 |
| 32.86 | 2.72 | 12.02 |
| 33.65 | 2.66 | 10.79 |
| 34.90 | 2.57 | 11.00 |

One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) dihydrochloride hydrate.

One aspect of the present invention is directed to a crystalline form of Compound 170 dihydrochloride hydrate, having a powder X-ray diffraction pattern comprising a peak, in terms of 2θ, at 24.23°±0.20°. One aspect of the present invention is directed to a crystalline form of Compound 170 dihydrochloride hydrate, having a powder X-ray diffraction pattern comprising a peak, in terms of 2θ, at 19.10°±0.20°. One aspect of the present invention is directed to a crystalline form of Compound 170 dihydrochloride hydrate, having a powder X-ray diffraction pattern comprising a peak, in terms of 2θ, at 11.71°±0.20°. One aspect of the present invention is directed to a crystalline form of Compound 170 dihydrochloride hydrate, having a powder X-ray diffraction pattern comprising peaks, in terms of 2θ, at 11.71°±0.20°, 19.10°±0.20°, and 24.23°±0.20°. One aspect of the present invention is directed to a crystalline form of Compound 170 dihydrochloride hydrate, having a powder X-ray diffraction pattern comprising peaks, in terms of 2θ, at 8.30°±0.20°, 11.71°±0.20°, 19.10°±0.20°, and 24.23°±0.20°. One aspect of the present invention is directed to a crystalline form of Compound 170 dihydrochloride hydrate, having a powder X-ray diffraction pattern comprising peaks, in terms of 2θ, at 8.30°±0.20°, 11.71°±0.20°, 15.54°±0.20°, 16.45°±0.20°, 19.10°±0.20°, and 24.23°±0.20°. One aspect of the present invention is directed to a crystalline form of Compound 170 dihydrochloride hydrate, having a powder X-ray diffraction pattern comprising peaks, in terms of 2θ, at 8.30°±0.20°, 11.71°±0.20°, 15.54°±0.20°, 16.45°±0.20°, 19.10°±0.20°, 21.64°±0.20°, and 24.23°±0.20°. One aspect of the present invention is directed to a crystalline form of Compound 170 dihydrochloride hydrate, having a powder X-ray diffraction pattern comprising one or more peaks listed in Table 6. One aspect of the present invention is directed to a crystalline form of Compound 170 dihydrochloride hydrate, having a powder X-ray diffraction pattern substantially as shown in FIG. 15, wherein by “substantially” is meant that the reported peaks can vary by about ±0.2 °2θ, and also that the relative intensities of the reported peaks can vary. One aspect of the present invention is directed to a crystalline form of Compound 170 dihydrochloride hydrate, having a thermogravimetric analysis profile substantially as shown in FIG. 16, wherein by “substantially” is meant that the reported TGA features can vary by about ±5° C. and by about ±2% weight change. One aspect of the present invention is directed to a crystalline form of Compound 170 dihydrochloride hydrate, having a dynamic moisture sorption profile substantially as shown in FIG. 17, wherein by “substantially” is meant that the reported DMS features can vary by about ±5% relative humidity and by about ±5% weight change.

C. Dihydrochloride Solvate

One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) dihydrochloride solvate. This dihydrochloride solvate can be characterized as a small-channel solvate and can be either a MeOH solvate or H₂O solvate (i.e., hydrate). Replacing MeOH with EtOH shifts the peaks in the PXRD pattern indicating a slight increase in the channel size. Certain solid-state properties of the crystalline form as prepared according to Example 2.8 are summarized in Table 7 below.

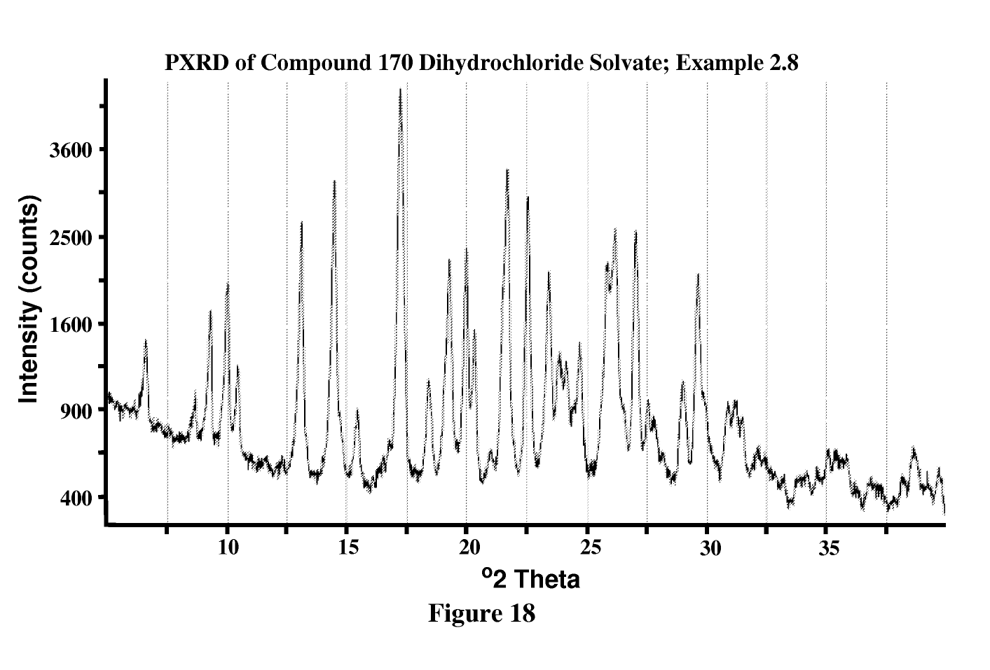

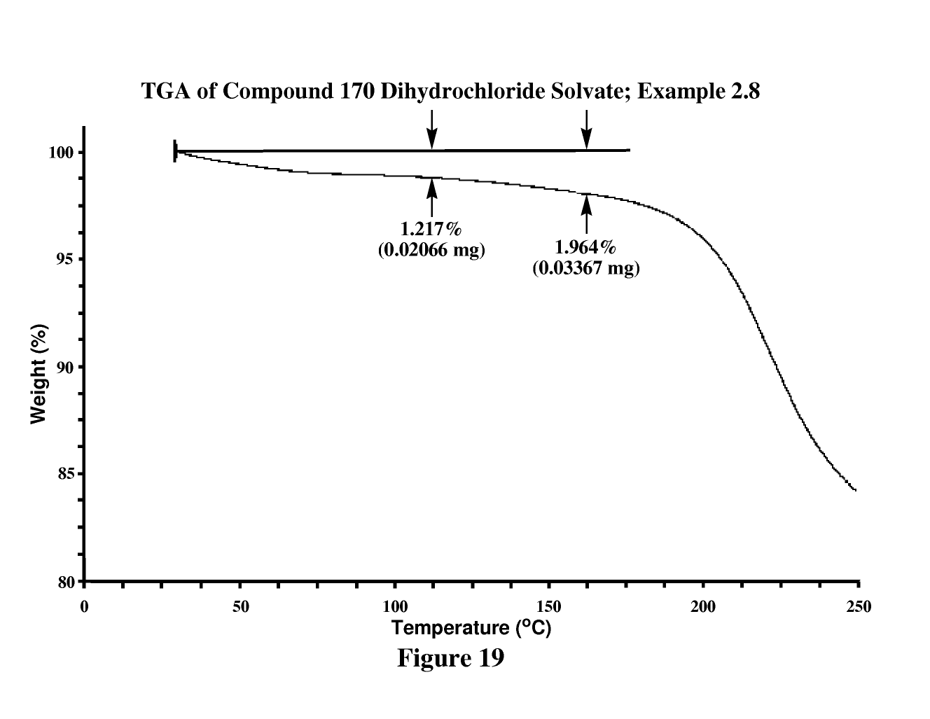

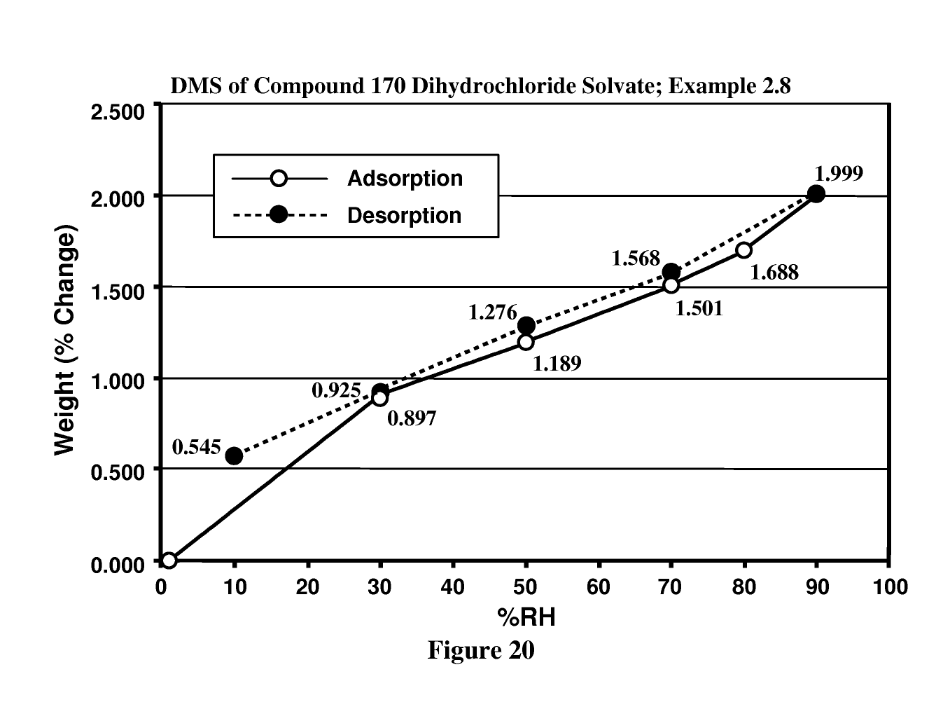

TABLE 7

| | Compound 170 dihydrochloride solvate |
|---|---|
| | (MeOH or H₂O); Example 2.8 |
| PXRD | FIG. 18: Peaks of about ≧15% relative intensity at 6.60, 9.30, |
| | 10.00, 10.45, 13.10, 14.47, 17.22, 18.40, 19.26, 19.97, 20.31, |
| | 21.68, 22.54, 23.41, 23.84, 24.15, 24.70, 25.86, 26.18, 27.04, |
| | 29.01, and 29.63 °2θ. A MeOH form that is indistinguishable |
| | by PXRD (± 0.2 °2θ) to the H₂O form was prepared in |
| | solvent containing ≧30% MeOH. |
| TGA | FIG. 19: Weight loss of about 1.2% out to about 112° C. |
| | (sample prepared in the absence of MeOH). |
| | Compound 170 dihydrochloride solvate (MeOH or H₂O); |
| | Example 2.8 |
| DMS | FIG. 20: The sample lost nearly 1% weight during the drying |
| | step at ~1% RH and 40° C. (Not shown in this isotherm plot). |
| | The total weight gain from the dried state was about 2%, thus |
| | the small-channel hydrate form typically holds about 1% water |
| | by weight under nominal conditions (30-50% RH and 25° C.), |
| | and can hold up to an additional ~1% at 90% RH and 25° C. |

Certain powder X-ray diffraction peaks for (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) dihydrochloride solvate are shown in Table 8 below.

TABLE 8

| | d-spacing | Rel. Int. |
|---|---|---|
| Pos. [°2θ.] | [Å] | [%] |
| 6.60 | 13.39 | 16.49 |
| 8.65 | 10.23 | 8.78 |
| 9.30 | 9.51 | 27.34 |
| 10.00 | 8.84 | 35.02 |
| 10.45 | 8.47 | 15.87 |
| 11.58 | 7.64 | 2.10 |
| 13.10 | 6.76 | 53.91 |
| 14.47 | 6.12 | 68.12 |
| 15.44 | 5.74 | 11.52 |
| 17.22 | 5.15 | 100.00 |
| 18.40 | 4.82 | 17.04 |
| 19.26 | 4.61 | 45.23 |
| 19.97 | 4.45 | 48.14 |
| 20.31 | 4.37 | 27.46 |
| 20.98 | 4.24 | 5.15 |
| 21.68 | 4.10 | 72.40 |
| 22.54 | 3.95 | 63.79 |
| 23.41 | 3.80 | 42.01 |
| 23.84 | 3.73 | 21.57 |
| 24.15 | 3.69 | 20.21 |
| 24.70 | 3.60 | 25.05 |
| 25.86 | 3.45 | 43.79 |
| 26.18 | 3.40 | 54.19 |
| 27.04 | 3.30 | 53.57 |
| 27.56 | 3.24 | 13.69 |
| 27.80 | 3.21 | 10.34 |
| 29.01 | 3.08 | 17.69 |
| 29.63 | 3.01 | 42.36 |
| 30.89 | 2.89 | 13.52 |
| 31.19 | 2.87 | 13.64 |
| 31.47 | 2.84 | 11.87 |
| 32.13 | 2.79 | 6.21 |
| 33.18 | 2.70 | 2.86 |
| 33.78 | 2.65 | 2.96 |
| 34.19 | 2.62 | 4.00 |
| 35.06 | 2.56 | 6.73 |
| 35.83 | 2.51 | 6.09 |
| 36.82 | 2.44 | 3.39 |
| 38.17 | 2.36 | 3.53 |
| 38.64 | 2.33 | 7.19 |

One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) dihydrochloride solvate. In some embodiments, the solvate is a hydrate. In some embodiments, the solvate is a MeOH solvate. In some embodiments, the solvate is an EtOH solvate.

One aspect of the present invention is directed to a crystalline form of Compound 170 dihydrochloride solvate, having a powder X-ray diffraction pattern comprising a peak, in terms of 2θ, at 17.22°±0.20°. One aspect of the present invention is directed to a crystalline form of Compound 170 dihydrochloride solvate, having a powder X-ray diffraction pattern comprising a peak, in terms of 2θ, at 14.47°±0.20°. One aspect of the present invention is directed to a crystalline form of Compound 170 dihydrochloride solvate, having a powder X-ray diffraction pattern comprising a peak, in terms of 2θ, at 13.10°±0.20°. One aspect of the present invention is directed to a crystalline form of Compound 170 dihydrochloride solvate, having a powder X-ray diffraction pattern comprising peaks, in terms of 2θ, at 13.10°±0.20°, 14.47°±0.20°, and 17.22°±0.20°. One aspect of the present invention is directed to a crystalline form of Compound 170 dihydrochloride solvate, having a powder X-ray diffraction pattern comprising peaks, in terms of 2θ, at 13.10°±0.20°, 14.47°±0.20°, 17.22°±0.20°, 21.68°±0.20°, and 22.54°±0.20°. One aspect of the present invention is directed to a crystalline form of Compound 170 dihydrochloride solvate, having a powder X-ray diffraction pattern comprising peaks, in terms of 2θ, at 13.10°±0.20°, 14.47°±0.20°, 17.22°±0.20°, 21.68°±0.20°, 22.54°±0.20°, 23.41°±0.20°, 27.04°±0.20°, and 29.63°±0.20°. One aspect of the present invention is directed to a crystalline form of Compound 170 dihydrochloride solvate, having a powder X-ray diffraction pattern comprising peaks, in terms of 2θ, at 13.10°±0.20°, 14.47°±0.20°, 17.22°±0.20°, 19.26°±0.20°, 19.97°±0.20°, 21.68°±0.20°, 22.54°±0.20°, 23.41°±0.20°, 27.04°±0.20°, and 29.63°±0.20°. One aspect of the present invention is directed to a crystalline form of Compound 170 dihydrochloride solvate, having a powder X-ray diffraction pattern comprising peaks, in terms of 2θ, at 9.30°±0.20°, 10.00°±0.20°, 13.10°±0.20°, 14.47°±0.20°, 17.22°±0.20°, 19.26°±0.20°, 19.97°±0.20°, 21.68°±0.20°, 22.54°±0.20°, 23.41°±0.20°, 26.18±0.20°, 27.04°±0.20°, and 29.63°±0.20°. One aspect of the present invention is directed to a crystalline form of Compound 170 dihydrochloride solvate, having a powder X-ray diffraction pattern comprising one or more peaks listed in Table 8. One aspect of the present invention is directed to a crystalline form of Compound 170 dihydrochloride solvate, having a powder X-ray diffraction pattern substantially as shown in FIG. 18, wherein by “substantially” is meant that the reported peaks can vary by about ±0.2 °2θ, and also that the relative intensities of the reported peaks can vary. One aspect of the present invention is directed to a crystalline form of Compound 170 dihydrochloride solvate, having a thermogravimetric analysis profile substantially as shown in FIG. 19, wherein by “substantially” is meant that the reported TGA features can vary by about ±5° C. and by about ±2% weight change. One aspect of the present invention is directed to a crystalline form of Compound 170 dihydrochloride solvate, having a dynamic moisture sorption profile substantially as shown in FIG. 20, wherein by “substantially” is meant that the reported DMS features can vary by about ±5% relative humidity and by about ±5% weight change.

### Sulfate Salt Solvate and Crystalline Form

One aspect of the present invention is directed to (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) sulfate solvate. Certain solid-state properties of the crystalline form of the sulfate solvate as prepared according to Example 2.9 are summarized in Table 9 below.

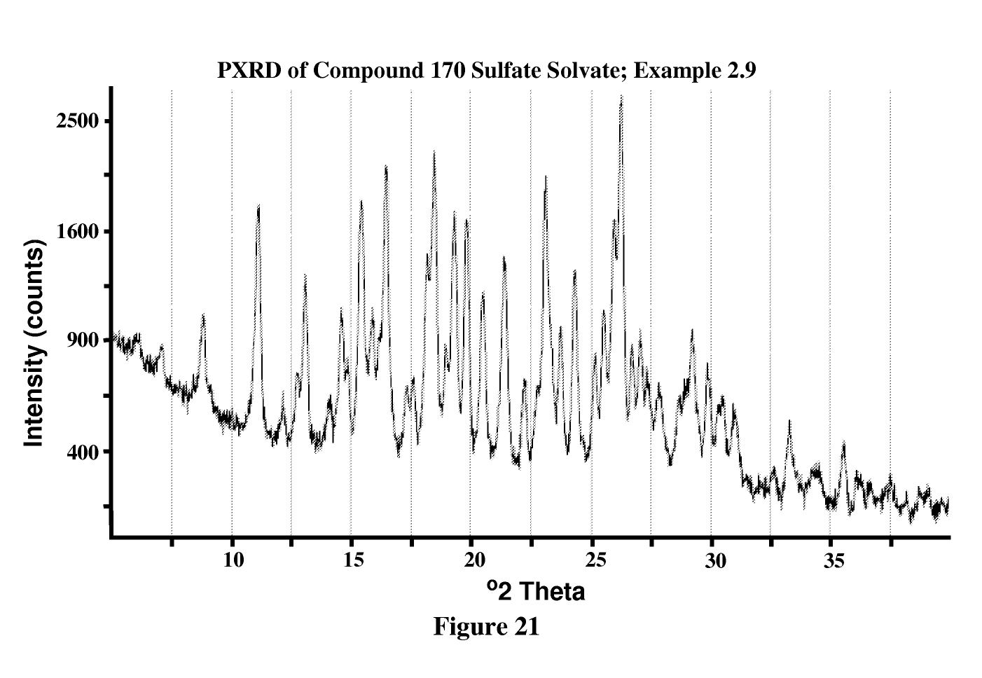

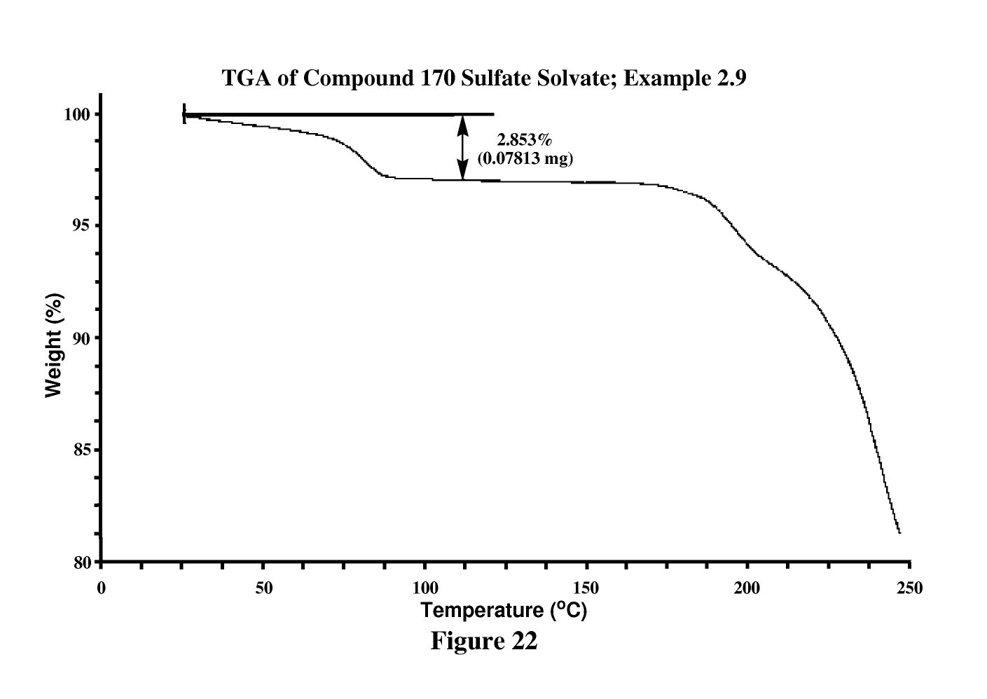

TABLE 9

| | Compound 170 sulfate solvate; Example 2.9 |
|---|---|
| PXRD | FIG. 21: Peaks of about ≧20% relative intensity at 8.82, 11.12, |
| | 13.09, 14.60, 15.42, 15.87, 16.46, 18.18, 18.47, 18.95, 19.31, |
| | 19.82, 20.50, 21.40, 23.12, 23.74, 24.34, 25.19, 25.54, 25.98, |
| | 26.26, 26.71, 27.07, and 29.22 °2θ. |
| TGA | FIG. 22: Weight loss of about 2.9% out to about 112° C. |

Certain powder X-ray diffraction peaks for (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) sulfate solvate are shown in Table 10 below.

TABLE 10

| | d-spacing | Rel. Int. |
|---|---|---|
| Pos. [°2θ.] | [Å] | [%] |
| 6.08 | 14.55 | 5.12 |
| 7.09 | 12.47 | 7.16 |
| 8.82 | 10.02 | 21.50 |
| 11.12 | 7.96 | 55.95 |
| 12.15 | 7.28 | 7.06 |
| 12.75 | 6.95 | 13.28 |
| 13.09 | 6.76 | 37.05 |
| 14.09 | 6.29 | 7.95 |
| 14.60 | 6.07 | 27.11 |
| 14.83 | 5.97 | 15.48 |
| 15.42 | 5.75 | 60.16 |
| 15.87 | 5.58 | 27.43 |
| 16.46 | 5.39 | 72.30 |
| 17.32 | 5.12 | 12.35 |
| 17.59 | 5.04 | 14.05 |
| 18.18 | 4.88 | 44.41 |
| 18.47 | 4.80 | 78.13 |
| 18.95 | 4.68 | 20.93 |
| 19.31 | 4.60 | 57.83 |
| 19.82 | 4.48 | 53.80 |
| 20.50 | 4.33 | 35.12 |
| 21.40 | 4.15 | 42.49 |
| 22.24 | 4.00 | 14.55 |
| 22.74 | 3.91 | 13.72 |
| 23.12 | 3.85 | 67.79 |
| 23.74 | 3.75 | 27.73 |
| 24.34 | 3.66 | 42.08 |
| 25.19 | 3.54 | 21.53 |
| 25.54 | 3.49 | 32.18 |
| 25.98 | 3.43 | 58.26 |
| 26.26 | 3.39 | 100.00 |
| 26.71 | 3.34 | 22.74 |
| 27.07 | 3.29 | 26.30 |
| 27.36 | 3.26 | 17.43 |
| 27.83 | 3.21 | 15.12 |
| 28.68 | 3.11 | 14.24 |
| 29.22 | 3.06 | 27.33 |
| 29.89 | 2.99 | 19.56 |
| 30.50 | 2.93 | 15.02 |
| 31.00 | 2.88 | 12.22 |
| 32.63 | 2.74 | 3.99 |
| 33.29 | 2.69 | 11.52 |
| 34.42 | 2.61 | 4.52 |
| 35.54 | 2.53 | 8.36 |
| 36.10 | 2.49 | 5.19 |
| 36.29 | 2.48 | 3.55 |
| 37.46 | 2.40 | 3.48 |
| 38.14 | 2.36 | 1.98 |

One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) sulfate solvate.

One aspect of the present invention is directed to a crystalline form of Compound 170 sulfate solvate, having a powder X-ray diffraction pattern comprising a peak, in terms of 2θ, at 26.26°±0.20°. One aspect of the present invention is directed to a crystalline form of Compound 170 sulfate solvate, having a powder X-ray diffraction pattern comprising a peak, in terms of 2θ, at 23.12°±0.20°. One aspect of the present invention is directed to a crystalline form of Compound 170 sulfate solvate, having a powder X-ray diffraction pattern comprising a peak, in terms of 2θ, at 11.12°±0.20°. One aspect of the present invention is directed to a crystalline form of Compound 170 sulfate solvate, having a powder X-ray diffraction pattern comprising peaks, in terms of 2θ, at 11.12°±0.20°, 23.12°±0.20°, and 26.26°±0.20°. One aspect of the present invention is directed to a crystalline form of Compound 170 sulfate solvate, having a powder X-ray diffraction pattern comprising peaks, in terms of 2θ, at 11.12°±0.20°, 15.42°±0.20°, 16.46°±0.20°, 23.12°±0.20°, and 26.26°±0.20°. One aspect of the present invention is directed to a crystalline form of Compound 170 sulfate solvate, having a powder X-ray diffraction pattern comprising peaks, in terms of 2θ, at 11.12°±0.20°, 15.42°±0.20°, 16.46°±0.20°, 18.47°±0.20°, 23.12°±0.20°, and 26.26°±0.20°. One aspect of the present invention is directed to a crystalline form of Compound 170 sulfate solvate, having a powder X-ray diffraction pattern comprising peaks, in terms of 2θ, at 11.12°±0.20°, 15.42°±0.20°, 16.46°±0.20°, 18.47°±0.20°, 19.31°±0.20°, 19.82°±0.20°, 23.12°±0.20°, 25.98°±0.20°, and 26.26°±0.20°. One aspect of the present invention is directed to a crystalline form of Compound 170 sulfate solvate, having a powder X-ray diffraction pattern comprising one or more peaks listed in Table 10. One aspect of the present invention is directed to a crystalline form of Compound 170 sulfate solvate, having a powder X-ray diffraction pattern substantially as shown in FIG. 21, wherein by “substantially” is meant that the reported peaks can vary by about ±0.2 °2θ and also that the relative intensities of the reported peaks can vary. One aspect of the present invention is directed to a crystalline form of Compound 170 sulfate solvate, having a thermogravimetric analysis profile substantially as shown in FIG. 22, wherein by “substantially” is meant that the reported TGA features can vary by about ±5° C. and by about ±2% weight change.

### Di-Mesylate and Crystalline Form

One aspect of the present invention is directed to (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) di-mesylate. One aspect of the present invention is directed to the crystalline form of (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) di-mesylate. The di-mesylate is an anhydrous crystalline form. Certain solid-state properties of the crystalline form of the di-mesylate are summarized in Table 11 below.

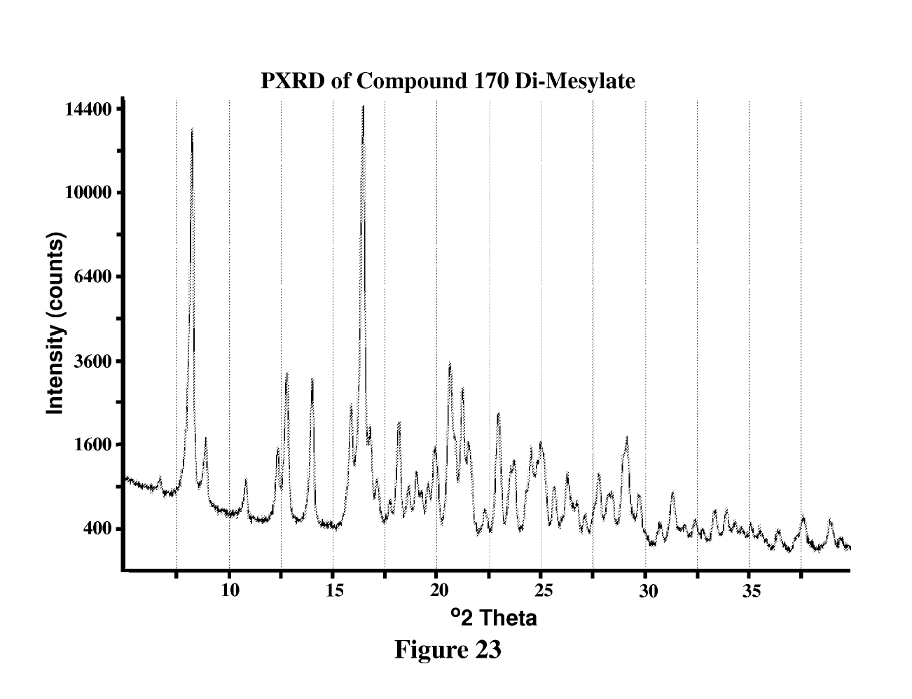

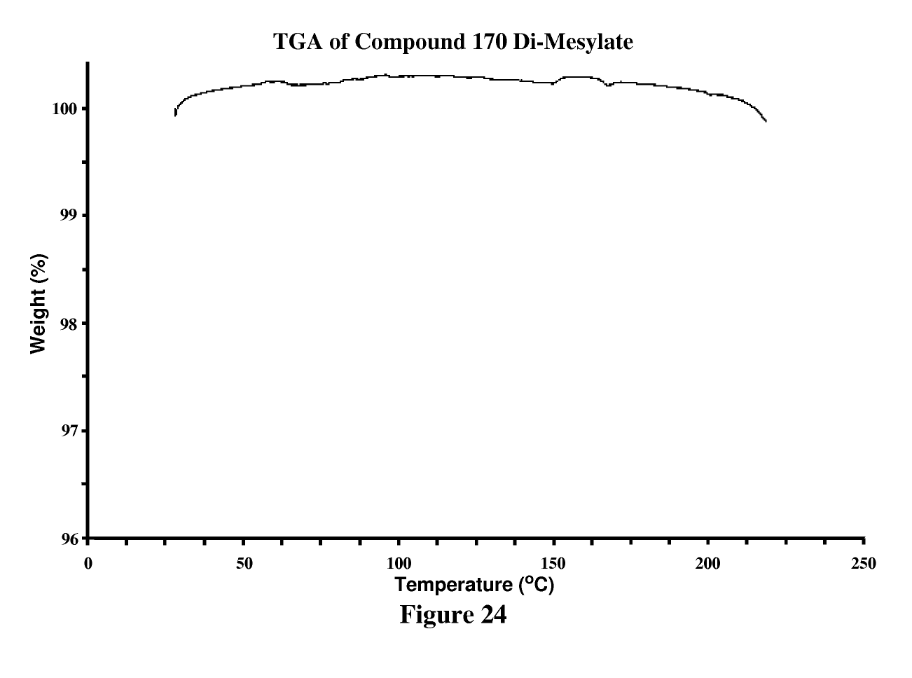

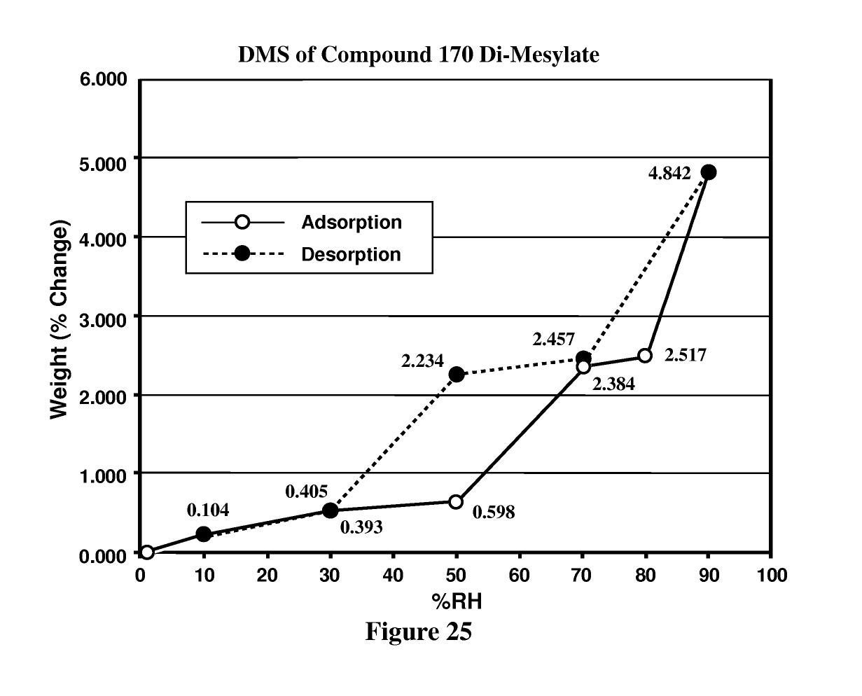

TABLE 11

| | Compound 170 di-mesylate |
|---|---|
| PXRD | FIG. 23: Peaks of about ≧15% relative intensity at 8.23, 12.78, |
| | 14.01, 15.88, 16.44, 20.63, 21.23, and 22.95 °2θ. |
| TGA | FIG. 24: No observed weight loss. |
| DMS | FIG. 25: The isotherm adsorption and desorption cycles show a |
| | plateau forming between 50 and 85% RH. This is likely a labile |
| | hydrate, as the weight gain matches the theoretical amount of |
| | water, 2.3%, for a monohydrate. The critical water activity of |
| | this proposed labile hydrate is between 0.3 and 0.7. |

Certain powder X-ray diffraction peaks for the crystalline form of (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) di-mesylate are shown in Table 12 below.

TABLE 12

| | d-spacing | Rel. Int. |
|---|---|---|
| Pos. [°2θ.] | [Å] | [%] |
| 8.23 | 10.74 | 88.54 |
| 8.88 | 9.95 | 7.96 |
| 12.35 | 7.17 | 7.63 |
| 12.78 | 6.93 | 19.76 |
| 14.01 | 6.32 | 19.01 |
| 15.88 | 5.58 | 14.78 |
| 16.44 | 5.39 | 100.00 |
| 16.79 | 5.28 | 11.34 |
| 18.17 | 4.88 | 11.94 |
| 19.01 | 4.67 | 5.14 |
| 19.89 | 4.46 | 7.69 |
| 20.63 | 4.31 | 22.72 |
| 20.85 | 4.26 | 9.85 |
| 21.23 | 4.19 | 17.73 |
| 21.51 | 4.13 | 9.23 |
| 21.65 | 4.10 | 5.83 |
| 22.95 | 3.88 | 13.59 |
| 23.51 | 3.78 | 5.51 |
| 23.70 | 3.75 | 7.08 |
| 24.52 | 3.63 | 8.30 |
| 24.98 | 3.56 | 9.52 |
| 25.14 | 3.54 | 7.42 |
| 26.27 | 3.39 | 5.16 |
| 27.77 | 3.21 | 5.63 |
| 28.93 | 3.09 | 7.81 |
| 29.12 | 3.07 | 10.31 |

One aspect of the present invention is directed to (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) di-mesylate.

One aspect of the present invention is directed to a crystalline form of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) di-mesylate.

One aspect of the present invention is directed to a crystalline form of Compound 170 di-mesylate, having a powder X-ray diffraction pattern comprising a peak, in terms of 2θ, at 16.44°±0.20°. One aspect of the present invention is directed to a crystalline form of Compound 170 di-mesylate, having a powder X-ray diffraction pattern comprising a peak, in terms of 2θ, at 8.23°±0.20°. One aspect of the present invention is directed to a crystalline form of Compound 170 di-mesylate, having a powder X-ray diffraction pattern comprising peaks, in terms of 2θ, at 8.23°±0.20° and 16.44°±0.20°. One aspect of the present invention is directed to a crystalline form of Compound 170 di-mesylate, having a powder X-ray diffraction pattern comprising peaks, in terms of 2θ, at 8.23°±0.20°, 12.78°±0.20°, 14.01°±0.20°, and 16.44°±0.20°. One aspect of the present invention is directed to a crystalline form of Compound 170 di-mesylate, having a powder X-ray diffraction pattern comprising peaks, in terms of 2θ, at 8.23°±0.20°, 12.78°±0.20°, 14.01°±0.20°, 16.44°±0.20°, 20.63°±0.20°, and 21.23°±0.20°. One aspect of the present invention is directed to a crystalline form of Compound 170 di-mesylate, having a powder X-ray diffraction pattern comprising peaks, in terms of 2θ, at 8.23°±0.20°, 12.78°±0.20°, 14.01°±0.20°, 16.44°±0.20°, 20.63°±0.20°, and 21.23°±0.20°. One aspect of the present invention is directed to a crystalline form of Compound 170 di-mesylate, having a powder X-ray diffraction pattern comprising peaks, in terms of 2θ, at 8.23°±0.20°, 12.78°±0.20°, 14.01°±0.20°, 15.88°±0.20°, 16.44°±0.20°, 20.63°±0.20°, 21.23°±0.20°, and 22.95°±0.20°. One aspect of the present invention is directed to a crystalline form of Compound 170 di-mesylate, having a powder X-ray diffraction pattern comprising one or more peaks listed in Table 12. One aspect of the present invention is directed to a crystalline form of Compound 170 di-mesylate, having a powder X-ray diffraction pattern substantially as shown in FIG. 23, wherein by “substantially” is meant that the reported peaks can vary by about ±0.2 °2θ, and also that the relative intensities of the reported peaks can vary. One aspect of the present invention is directed to a crystalline form of Compound 170 di-mesylate, having a thermogravimetric analysis profile substantially as shown in FIG. 24, wherein by “substantially” is meant that the reported TGA features can vary by about ±5° C. and by about ±2% weight change. One aspect of the present invention is directed to a crystalline form of Compound 170 di-mesylate, having a dynamic moisture sorption profile substantially as shown in FIG. 25, wherein by “substantially” is meant that the reported DMS features can vary by about ±5% relative humidity and by about ±5% weight change.

The crystalline forms described herein can be prepared by any of the suitable procedures known in the art for preparing crystalline polymorphs. In some embodiments the crystalline forms described herein are prepared according to the Examples.

### Other Utilities

Another object of the present invention relates to radiolabeled compounds of the present invention that would be useful not only in radio-imaging but also in assays, both in vitro and in vivo, for localizing and quantitating Mas receptors in tissue samples, including human and for identifying Mas receptor ligands by inhibition binding of a radiolabeled compound. It is a further object of this invention to develop novel Mas receptor assays of which comprise such radiolabeled compounds.

The present disclosure includes all isotopes of atoms occurring in the present compounds, intermediates, salts and crystalline forms thereof. Isotopes include those atoms having the same atomic number but different mass numbers. One aspect of the present invention includes every combination of one or more atoms in the present compounds, intermediates, salts, and crystalline forms thereof that is replaced with an atom having the same atomic number but a different mass number. One such example is the replacement of an atom that is the most naturally abundant isotope, such as ¹H or ¹²C, found in one the present compounds, intermediates, salts, and crystalline forms thereof, with a different atom that is not the most naturally abundant isotope, such as ²H or ³H (replacing ¹H), or ¹¹C, ¹³C, or ¹⁴C (replacing ¹²C). A compound wherein such a replacement has taken place is commonly referred to as being an isotopically-labeled compound. Isotopic-labeling of the present compounds, intermediates, salts, and crystalline forms thereof can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. Isotope of fluorine include ¹⁸F. Isotopes of phosphorous include ³²P and ³³P. Isotopes of sulfur include ³⁵S. Isotopes of chlorine include ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present compounds, intermediates, salts, and crystalline forms thereof, wherein the naturally occurring distribution of the isotopes in the composition is perturbed. Another aspect of the present invention includes compositions and pharmaceutical compositions comprising compounds as described herein wherein the compound is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Drawings and Examples by substituting an isotopically labeled reagent for a non-isotopically labeled reagent. Other synthetic methods that are useful are discussed below. Moreover, it should be understood that all of the atoms represented in the compounds of the invention can be either the most commonly occurring isotope of such atoms or a scarcer radio-isotope or nonradioactive isotope.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas: This procedure normally yields high specific activity products and requires halogenated or unsaturated precursors.

B. Reduction with Sodium Borohydride [³H]: This procedure is rather inexpensive and requires precursors containing reducible functional groups such as aldehydes, ketones, lactones, esters and the like.

C. Reduction with Lithium Aluminum Hydride [³H]: This procedure offers products at almost theoretical specific activities. It also requires precursors containing reducible functional groups such as aldehydes, ketones, lactones, esters and the like.

D. Tritium Gas Exposure Labeling: This procedure involves exposing precursors containing exchangeable protons to tritium gas in the presence of a suitable catalyst.

E. N-Methylation using Methyl Iodide [³H]: This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A representative procedure was reported by Zhu, G-D. and co-workers in J. Org. Chem., 2002, 67, 943-948.

B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.

C. Aryl and heteroaryl bromide exchange with ¹²⁵I: This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm. 2001, 44, S280-S282.

A radiolabeled form of a compound of Formula (I) can be used in a screening assay to identify/evaluate compounds. In general terms, a newly synthesized or identified compound (i.e., test compound) can be evaluated for its ability to reduce binding of a radiolabeled form of a compound of Formula (I) to a Mas receptor. The ability of a test compound to compete with a radiolabeled form of a compound of Formula (I) for the binding to a Mas receptor directly correlates to its binding affinity.

Certain labeled compounds of the present invention bind to certain Mas receptors. In one embodiment the labeled compound has an IC₅₀ less than about 500 μM. In one embodiment the labeled compound has an IC₅₀ less than about 100 μM. In one embodiment the labeled compound has an IC₅₀ less than about 10 μM. In one embodiment the labeled compound has an IC₅₀ less than about 1 μM. In one embodiment the labeled compound has an IC₅₀ less than about 0.1 μM. In one embodiment the labeled compound has an IC₅₀ less than about 0.01 μM. In one embodiment the labeled compound has an IC₅₀ less than about 0.005 μM.

Other uses of the disclosed receptors and methods will become apparent to those skilled in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### EXAMPLES

### Example 1

### Syntheses of Compounds of the Present Invention

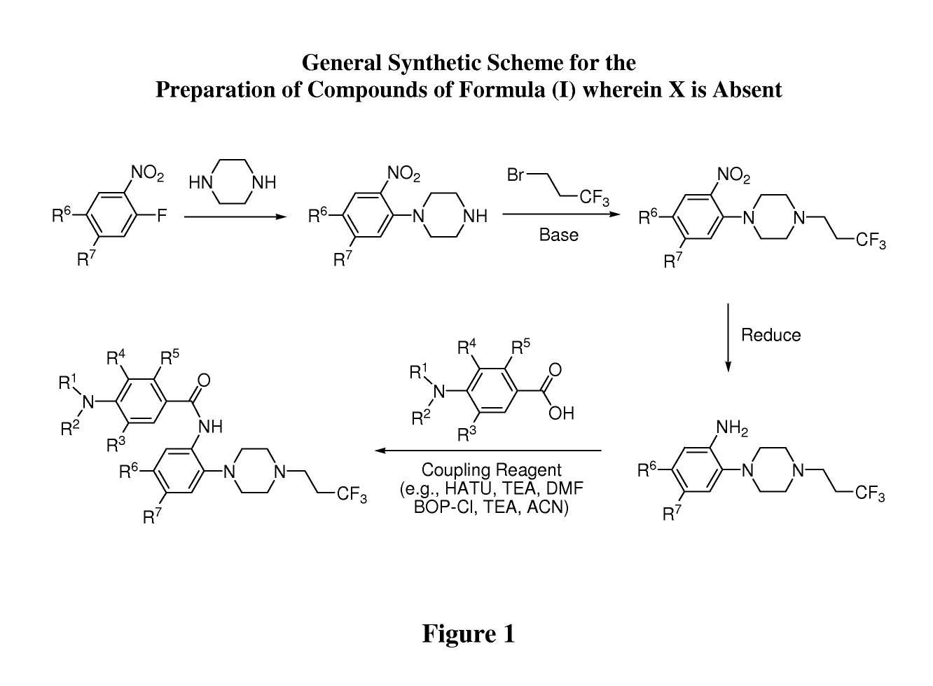

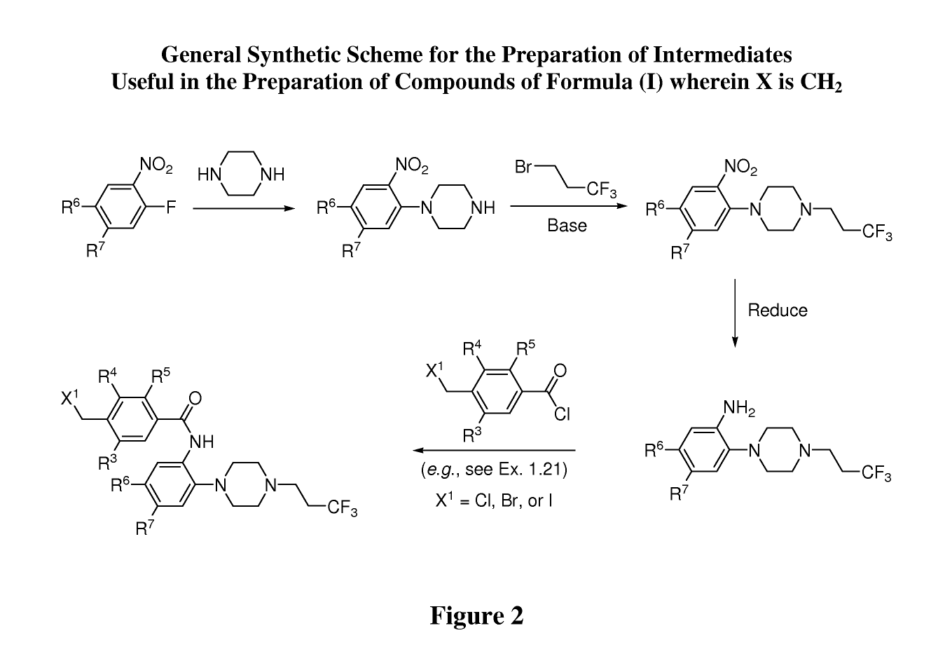

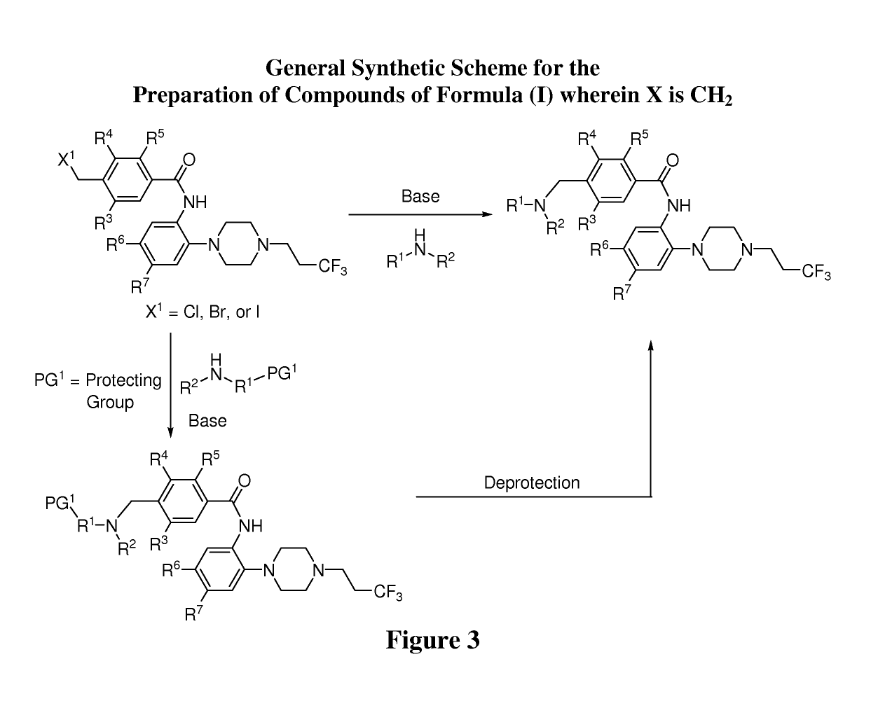

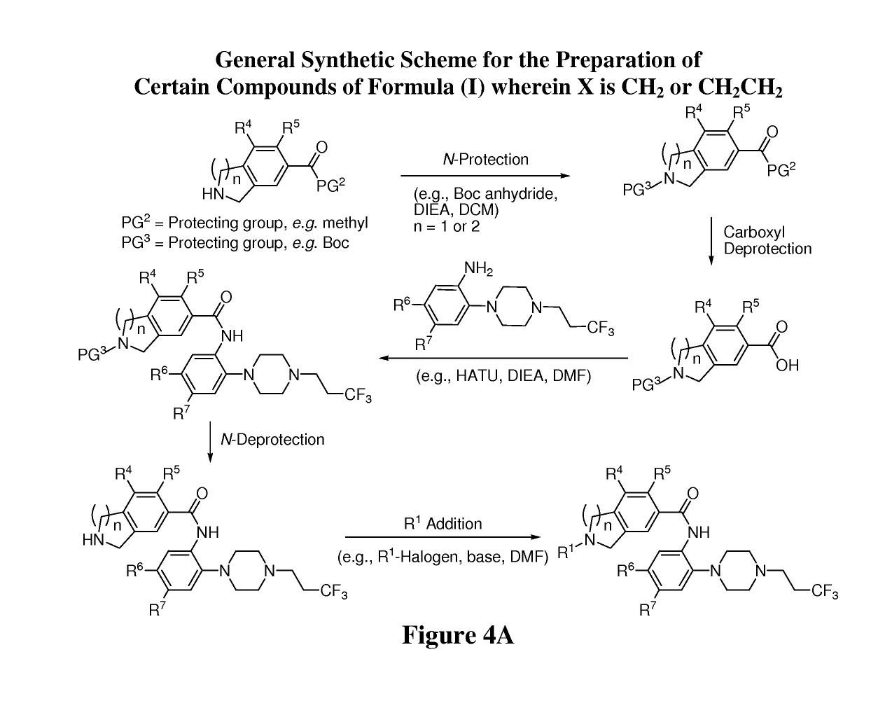

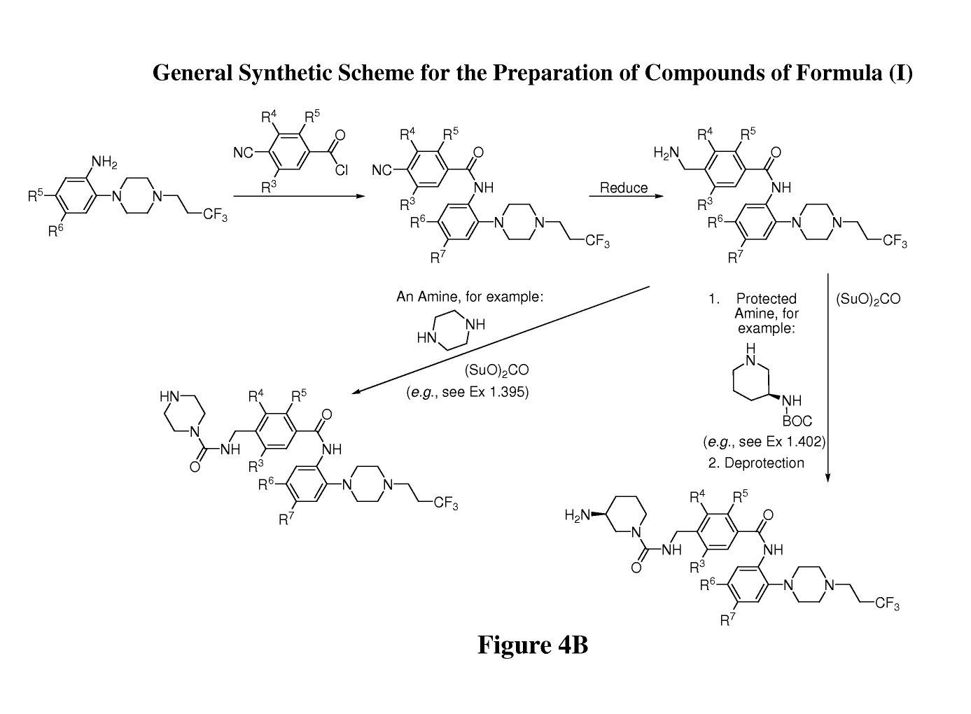

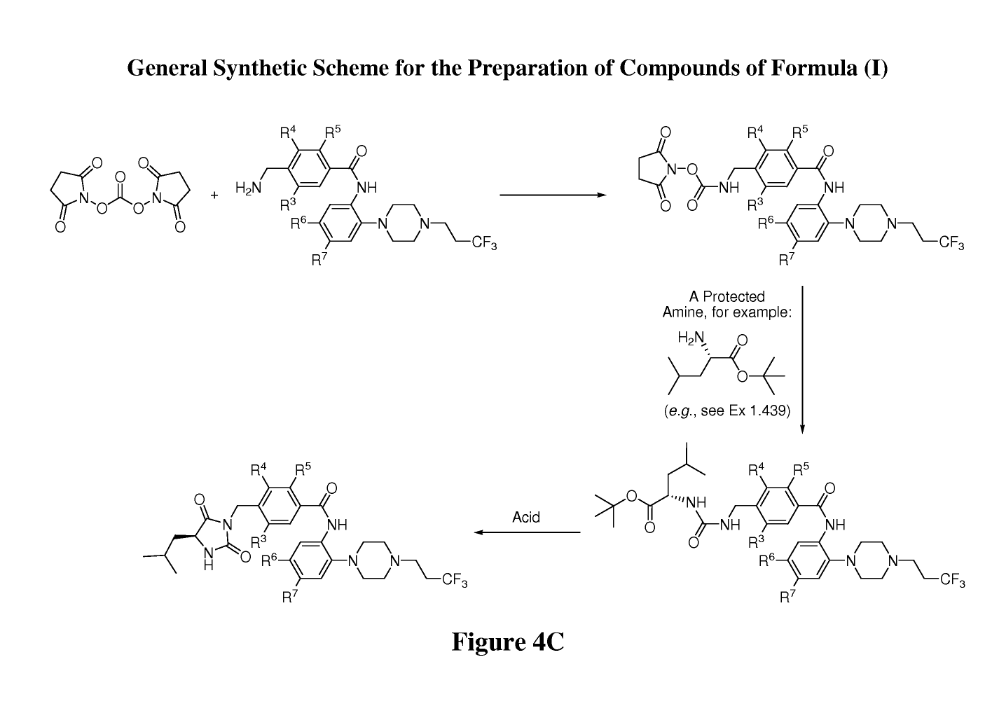

Illustrated syntheses for compounds of the present invention are shown in FIG. 1 to FIG. 4C wherein the variables R¹, R², R³, R⁴, R⁵, R⁶, R⁷, and “X” have the same definitions as used throughout this disclosure.

The compounds of the invention and their syntheses are further illustrated by the following examples. The following examples are provided to further define the invention without, however, limiting the invention to the particulars of these examples. The compounds described herein, supra and infra, are named according to AutoNom version 2.2, AutoNom 2000, CS ChemDraw Ultra Version 7.0.1, or CS ChemDraw Ultra Version 9.0.7. In certain instances common names are used and it is understood that these common names would be recognized by those skilled in the art.

Proton nuclear magnetic resonance (¹H NMR) spectra were recorded on a Bruker Avance-400 equipped with a QNP (Quad Nucleus Probe) or a BBI (Broad Band Inverse) and z-gradient. Chemical shifts are given in parts per million (ppm) with the residual solvent signal used as reference. NMR abbreviations are used as follows: s=singlet, d=doublet, dd=doublet of doublets, ddd=doublet of doublet of doublets, dt=doublet of triplets, t=triplet, td=triplet of doublets, tt=triplet of triplets, q=quartet, m=multiplet, br=broad, bs=broad singlet, bt=broad triplet. Microwave irradiations were carried out using a Smith Synthesizer™ or an Emrys Optimizer™ (Biotage). Thin-layer chromatography (TLC) was performed on silica gel 60 F254 (Merck), preparatory thin-layer chromatography (prep TLC) was preformed on PK6F silica gel 60 A 1 mm plates (Whatman) and column chromatography was carried out on a silica gel column using Kieselgel 60, 0.063-0.200 mm (Merck). Evaporation was done under reduced pressure on a Biichi rotary evaporator.

LCMS spec: HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: API 150EX with Turbo Ion Spray source, AB/MDS Sciex; Software: Analyst 1.2.

### Example 1.1

### Preparation of N-(4-Chloro-2-(4-(3,3,3-trifluoropropyl)piperazin-1-yl)phenyl)-4-((diethylamino)methyl)benzamide (Compound 2)

### Step A: Preparation of 4-Chloro-2-(4-(3,3,3-trifluoropropyl)piperazin-1-yl)aniline

Piperazine (36.8 g, 427 mmol) was dissolved in IPA (150 mL) and cooled in an ice bath. 4-Chloro-2-fluoro-1-nitrobenzene (25 g, 142 mmol), pre-dissolved in IPA (100 mL), was added to the reaction slowly via addition funnel (the solution turned yellowish orange over time). Upon complete addition, the reaction was warmed to room temperature and stirred at this temperature overnight. The next day, the solvent was evaporated and the product was extracted (200 mL each of H₂O and EtOAc). The aqueous layer was extracted twice more with EtOAc (200 mL). The organic layers were combined and back extracted once with H₂O/brine (500 mL). The organic layer was dried over MgSO₄ and concentrated to give 1-(5-chloro-2-nitrophenyl)piperazine as a reddish oil. This material was dissolved in THF (50 mL) and MeOH (10 mL). DIEA (49.7 mL, 285 mmol) and 3-bromo-1,1,1-trifluoropropane (22.84 mL, 214 mmol) were added. The reaction was heated to reflux in an oil bath and stirred at this temperature overnight. The next day, the reaction was around 70% complete. Thus, more 3-bromo-1,1,1-trifluoropropane (10 mL) and DIEA (20 mL) were added and the reaction was heated to reflux again overnight. The solvent was evaporated to give 1-(5-chloro-2-nitrophenyl)-4-(3,3,3-trifluoropropyl)piperazine, as a reddish-yellow waxy solid. The solid was dissolved in EtOH (150 mL) and the reaction was cooled on an ice bath. To the stirred solution was added SnCl₂ (81 g, 427 mmol) portionwise (in 10 g portions; allowing the tin chloride to fully dissolve and the reaction to cool in between). Upon complete addition, the reaction was heated to 80° C. in an oil bath and stirred at this temperature for 1 h. After this time, the reaction was cooled on an ice bath. Conc. NaOH (50 wt %) was added portionwise (in ˜20 mL portions). To the reaction were added CH₂Cl₂, and H₂O (˜1.2 L each), and the layers were separated. The aqueous layer was extracted twice more with CH₂Cl₂ (2×1 L). The organic layers were combined, dried, and concentrated. The residue was purified by column chromatography to give the title compound (42.7 g, 94%), as a light yellowish tan solid. LC/MS m/z=308.2 [M+H]⁺; ¹H NMR (400 MHz, DMSO-d₆) δ ppm 2.43-2.66 (m, 8H), 2.76-2.89 (m, 4H), 4.81-4.82 (m, 2H), 6.69 (d, J=8.21 Hz, 1H), 6.82-6.89 (m, 2H).

### Step B: Preparation of N-(4-Chloro-2-(4-(3,3,3-trifluoropropyl)piperazin-1-yl)phenyl)-4-(chloromethyl)benzamide

4-(Chloromethyl)benzoyl chloride (160 mg, 0.845 mmol) was dissolved in CH₂Cl₂ (2 mL) and cooled in an ice bath. 4-Chloro-2-(4-(3,3,3-trifluoropropyl)piperazin-1-yl)aniline (200 mg, 0.650 mmol) and DIEA (170 μL, 0.975 mmol) were pre-dissolved in CH₂Cl₂ (1 mL), then added slowly to the solution on ice. Upon complete addition, the reaction was warmed to room temperature. After stirring for 30 min, the solvent was evaporated and the residue was purified by column chromatography to give the title compound (291 mg, 95%), as a white solid. LC/MS m/z=460.4 [M+H]⁺; ¹H NMR (400 MHz, DMSO-d₆) δ ppm 2.43-2.64 (m, 8H), 2.85-2.95 (m, 4H), 4.86 (s, 2H), 7.20 (dd, J=8.65, 2.34 Hz, 1H), 7.28 (d, J=2.40 Hz, 1H), 7.64 (d, J=8.21 Hz, 2H), 7.95 (d, J=8.21 Hz, 2H), 8.03 (d, J=8.59 Hz, 1H), 9.52 (s, 1H).

### Step C: Preparation of N-(4-Chloro-2-(4-(3,3,3-trifluoropropyl)piperazin-1-yl)phenyl)-4-((diethylamino)methyl)benzamide (Compound 2) as the bis(2,2,2-trifluoroactetate salt

N-(4-Chloro-2-(4-(3,3,3-trifluoropropyl)piperazin-1-yl)phenyl)-4-(chloromethyl)benzamide (20 mg, 0.043 mmol), DIEA (11.38 μL, 0.065 mmol), and diethylamine (22.70 μL, 0.217 mmol) were dissolved in DMF (0.4 mL). The reaction was heated to 80° C. for 1 h, and then the reaction was stirred at room temperature overnight. The next day, the reaction was complete. The mixture was purified by preparative LC/MS (10-95% ACN/H₂O (0.1% TFA), 30 min) to give the title compound (25.2 mg, 80%) as a white solid (TFA salt). LC/MS m/z=497.4 [M+H]⁺; ¹H NMR (400 MHz, CDCl₃) δ ppm 1.43 (t, J=7.26 Hz, 6H), 2.64-2.80 (m, 2H), 3.11-3.39 (m, 8H), 3.40-3.47 (m, 4H), 3.7-3.48 (bs, 2H), 4.15-4.24 (m, 2H), 7.21-7.30 (m, 2H), 7.58 (d, J=8.21 Hz, 2H), 7.88 (d, J=8.08 Hz, 2H), 8.51 (d, J=8.59 Hz, 1H), 9.09 (s, 1H), 10.66 (bs, 1H).

### Examples 1.2 to 1.11, and 1.183

The following compounds were prepared using the disclosed intermediates and a method similar to the one described in Example 1.1, Step C.

| | | | Product |
|---|---|---|---|
| Example | Cmpd | | (Mass Observed |
| No. | No. | Intermediates Used | LC/MS m/z) |
| 1.2 | 3 | N-(4-chloro-2-(4-(3,3,3-trifluoropropyl)piperazin- | 485.4 [M + H]⁺ |
| | | 1-yl)phenyl)-4-(chloromethyl)benzamide and 2- | |
| | | aminoethanol | |
| 1.3 | 4 | N-(4-chloro-2-(4-(3,3,3-trifluoropropyl)piperazin- | 469.4 [M + H]⁺ |
| | | 1-yl)phenyl)-4-(chloromethyl)benzamide and | |
| | | ethanamine | |
| 1.4 | 5 | N-(4-chloro-2-(4-(3,3,3-trifluoropropyl)piperazin- | 510.8 [M + H]⁺ |
| | | 1-yl)phenyl)-4-(chloromethyl)benzamide and (S)- | |
| | | pyrrolidin-3-ol | |
| 1.5 | 6 | N-(4-chloro-2-(4-(3,3,3-trifluoropropyl)piperazin- | 554.4 [M + H]⁺ |
| | | 1-yl)phenyl)-4-(chloromethyl)benzamide and 2- | |
| | | (piperazin-1-yl)ethanol | |
| 1.6 | 7 | N-(4-chloro-2-(4-(3,3,3-trifluoropropyl)piperazin- | 550.0 [M + H]⁺ |
| | | 1-yl)phenyl)-4-(chloromethyl)benzamide and (S)- | |
| | | octahydropyrrolo[1,2-a]pyrazine | |
| 1.7 | 8 | N-(4-chloro-2-(4-(3,3,3-trifluoropropyl)piperazin- | 499.4 [M + H]⁺ |
| | | 1-yl)phenyl)-4-(chloromethyl)benzamide and 2- | |
| | | (methylamino)ethanol | |
| 1.8 | 1 | N-(4-chloro-2-(4-(3,3,3-trifluoropropyl)piperazin- | 559.2 [M + H]⁺ |
| | | 1-yl)phenyl)-4-(chloromethyl)benzamide and | |
| | | thiomorpholin-1,1-dioxide | |
| 1.9 | 9 | N-(4-chloro-2-(4-(3,3,3-trifluoropropyl)piperazin- | 538.0 [M + H]⁺ |
| | | 1-yl)phenyl)-4-(chloromethyl)benzamide and 1- | |
| | | ethylpiperazine | |
| 1.10 | 10 | N-(4-chloro-2-(4-(3,3,3-trifluoropropyl)piperazin- | 549.2 [M + H]⁺ |
| | | 1-yl)phenyl)-4-(chloromethyl)benzamide and 3- | |
| | | (1H-imidazol-1-yl)propan-1-amine | |
| 1.11 | 11 | N-(4-chloro-2-(4-(3,3,3-trifluoropropyl)piperazin- | 532.2 [M + H]⁺ |
| | | 1-yl)phenyl)-4-(chloromethyl)benzamide and 4- | |
| | | methylpyridin-3-amine | |
| 1.183 | 12 | N-(4-chloro-2-(4-(3,3,3-trifluoropropyl)piperazin- | 509.2 [M + H]⁺ |
| | | 1-yl)phenyl)-4-(chloromethyl)benzamide and | |
| | | piperidine | |

### Example 1.12

### Preparation of N-(4-Chloro-2-(4-(3,3,3-trifluoropropyl)piperazin-1-yl)phenyl)isoindoline-5-carboxamide (Compound 25)

To a solution of 4-chloro-2-(4-(3,3,3-trifluoropropyl)piperazin-1-yl)aniline (0.25 g, 0.812 mmol) in DMF (2 mL), were added 2-(tert-butoxycarbonyl)isoindoline-5-carboxylic acid (0.214 g, 0.812 mmol), HATU (0.309 g, 0.812 mmol), and DIEA (0.105 g, 0.812 mmol) at ambient temperature. After stirring for 12 h, the reaction was extracted with ethyl acetate. The ethyl acetate was dried over MgSO₄ and concentrated. The residue was treated with 4.0 M HCl in dioxane (1 mL) for 5 h and concentrated under reduced pressure to give the title compound (0.25 g, 67.9%). LC/MS m/z=453.2 [M+H]⁺.

### Example 1.13

### Preparation of Ethyl 2-(5-(4-Chloro-2-(4-(3,3,3-trifluoropropyl)piperazin-1-yl)phenylcarbamoyl)isoindolin-2-yl)acetate (Compound 27)

N-(4-Chloro-2-(4-(3,3,3-trifluoropropyl)piperazin-1-yl)phenyl)isoindoline-5-carboxamide dihydrochloride (10 mg, 0.019 mmol), DIEA (33.2 μL, 0.190 mmol), and ethyl bromoacetate (3.8 mg, 0.023 mmol) were added to a vial with DMF (0.2 mL). The reaction was heated to 80° C. for 1 h. The mixture was purified by preparative LC/MS (5-70% ACN/H₂O (0.1% TFA), 25 min) to give the title compound (4.4 mg, 30%), as a TFA salt. LC/MS m/z=539.2 [M+H]⁺; ¹H NMR (400 MHz, DMSO-d₆) δ ppm 1.27 (t, J=7.14 Hz, 3H), 2.75-2.91 (m, 2H), 3.02-3.66 (m, 12H), 4.24 (q, J=7.16 Hz, 2H), 4.31-4.43 (m, 2H), 4.60-4.72 (m, 2H), 7.25 (dd, J=8.59, 2.15 Hz, 1H), 7.32 (d, J=2.40 Hz, 1H), 7.56 (d, J=7.83 Hz, 1H), 7.91-7.98 (m, 3H), 9.54 (s, 1H).

### Examples 1.14 to 1.19

The following compounds were prepared using the disclosed intermediates and a method similar to the one described in Example 1.13.

| | | | Product |
|---|---|---|---|
| Example | Cmpd | | (Mass Observed |
| No. | No. | Intermediates Used | LC/MS m/z) |
| 1.14 | 28 | N-(4-chloro-2-(4-(3,3,3-trifluoropropyl)piperazin- | 497.4 [M + H]⁺ |
| | | 1-yl)phenyl)isoindoline-5-carboxamide | |
| | | dihydrochloride and 2-bromoethanol | |
| 1.15 | 29¹ | N-(4-chloro-2-(4-(3,3,3-trifluoropropyl)piperazin- | 492.0 [M + H]⁺ |
| | | 1-yl)phenyl)isoindoline-5-carboxamide | |
| | | dihydrochloride and 2-bromoacetonitrile | |
| 1.16 | 30 | N-(4-chloro-2-(4-(3,3,3-trifluoropropyl)piperazin- | 510.2 [M + H]⁺ |
| | | 1-yl)phenyl)isoindoline-5-carboxamide | |
| | | dihydrochloride and 2-bromoacetamide | |
| 1.17 | 31 | N-(4-chloro-2-(4-(3,3,3-trifluoropropyl)piperazin- | 525.6 [M + H]⁺ |
| | | 1-yl)phenyl)isoindoline-5-carboxamide | |
| | | dihydrochloride and 1-bromo-2-ethoxyethane | |
| 1.18 | 32 | N-(4-chloro-2-(4-(3,3,3-trifluoropropyl)piperazin- | 525.6 [M + H]⁺ |
| | | 1-yl)phenyl)isoindoline-5-carboxamide | |
| | | dihydrochloride and 2-bromo-N,N- | |
| | | diethylethanamine hydrobromide | |
| 1.19 | 33 | N-(4-chloro-2-(4-(3,3,3-trifluoropropyl)piperazin- | 524.2 [M + H]⁺ |
| | | 1-yl)phenyl)isoindoline-5-carboxamide | |
| | | dihydrochloride and 3-bromopropanamide | |

¹Compound 29: ¹H NMR (400 MHz, DMSO-d₆) δ ppm 2.80-2.92 (m, 2H), 3.02-3.84 (m, 9H), 4.06 (d, J = 7.33 Hz, 7H), 7.25 (dd, J = 8.65, 2.34 Hz, 1H), 7.32 (d, J = 2.27 Hz, 1H), 7.47 (d, J = 7.83 Hz, 1H), 7.83-7.89 (m, 2H), 7.95 (d, J = 8.72 Hz, 1H), 9.46 (s, 1H).

### Example 1.20

### Preparation of N-(4-Chloro-2-(4-(3,3,3-trifluoropropyl)piperazin-1-yl)phenyl)-4-(chloromethyl)-2,3-difluorobenzamide and 4-(Bromomethyl)-N-(4-chloro-2-(4-(3,3,3-trifluoropropyl)piperazin-1-yl)phenyl)-2,3-difluorobenzamide (Intermediate 1)

### Step A: Preparation of 4-(Bromomethyl)-2,3-difluorobenzoic acid

2,3-Difluoro-4-methylbenzoic acid (18 g, 105 mmol) and NBS (18.61 g, 105 mmol) were added to a round bottom flask with CCl₄ (550 mL). AIBN (0.086 g, 0.523 mmol) was added and the reaction was heated to reflux (90° C.) and stirred at this temperature overnight. The next day, the reaction was cooled to room temperature. The resulting mixture was filtered and washed with DCM. The filtrate was concentrated, diluted with DCM (200 mL) and then extracted with H₂O (200 mL). The organic layers were combined, dried, and concentrated to give the title compound (23.5 g, 44.8%). Exact mass calculated for C₈H₅BrF₂O₂: 249.9. found: LCMS m/z=250.2 [M+H]⁺; ¹H NMR (400 MHz, DMSO-d₆) δ ppm 4.76 (s, 2H), 7.42-7.48 (m, 1H), 7.64-7.70 (m, 1H).

### Step B: Preparation of a mixture of N-(4-Chloro-2-(4-(3,3,3-trifluoropropyl)piperazin-1-yl)phenyl)-4-(chloromethyl)-2,3-difluorobenzamide and 4-(Bromomethyl)-N-(4-chloro-2-(4-(3,3,3-trifluoropropyl)piperazin-1-yl)phenyl)-2,3-difluorobenzamide

4-(Bromomethyl)-2,3-difluorobenzoic acid (10 g, 39.8 mmol) was suspended in DCM (60 mL). Oxalyl chloride (3.49 mL, 39.8 mmol) was added at room temperature, followed by DMF (100 μL). The reaction was stirred at room temperature for 1 h. Then the solvent was removed and the resulting dark-purple oil was re-dissolved in DCM (50 mL). The mixture was cooled on ice and DIEA (13.92 mL, 80 mmol) and 4-chloro-2-(4-(3,3,3-trifluoropropyl)piperazin-1-yl)aniline (12.26 g, 39.8 mmol) were added. The reaction was warmed to room temperature and stirred for 1 h. The resulting reaction mixture was purified by column chromatography (0, 5, 10% EtOAc/hexanes) to give the title compounds (9.04 g, 45.7%). LCMS (peak 1) m/z=540.3 [M+H]⁺, (peak 2) m/z=496.3 [M+H]⁺; ¹H NMR (400 MHz, DMSO-d₆) δ ppm 2.45-2.55 (m, 2H), 2.54-2.68 (m, 6H), 2.89 (t, J=4.42 Hz, 4H), 4.79 (s, 0.5H), 4.91 (s, 1.5H), 7.23 (dd, J=8.72, 2.27 Hz, 1H), 7.33 (d, J=1.89 Hz, 1H), 7.53 (t, J=6.88 Hz, 1H), 7.71 (q, J=7.37 Hz, 1H), 8.20 (d, J=8.59 Hz, 1H), 9.78 (d, J=6.95 Hz, 1H).

### Example 1.21

### Preparation of N-(4-Chloro-2-(4-(3,3,3-trifluoropropyl)piperazin-1-yl)phenyl)-4-((cyanomethylamino)methyl)-2-fluorobenzamide (Compound 65)

### Step A: Preparation of 4-(Bromomethyl)-2-fluorobenzoic Acid

To a solution of 2-fluoro-4-methylbenzoic acid (10 g, 64.9 mmol) in CCl₄ (50 mL), was added NBS (11.55 g, 64.9 mmol) followed by AIBN (0.053 g, 0.324 mmol) at room temperature. The reaction was refluxed at 100° C. for 3 h. After cooling to room temperature, the precipitate was filtered and washed with hexane to give the title compound (6.8 g, 45.0%) which was used in the next step without further purification.

### Step B: Preparation of a mixture of 4-(Bromomethyl)-N-(4-chloro-2-(4-(3,3,3-trifluoropropyl)piperazin-1-yl)phenyl)-2-fluorobenzamide and N-(4-Chloro-2-(4-(3,3,3-trifluoropropyl)piperazin-1-yl)phenyl)-4-(chloromethyl)-2-fluorobenzamide (Intermediate 2)

To a solution of 4-(bromomethyl)-2-fluorobenzoic acid (0.76 g, 3.25 mmol) in CH₂Cl₂ (5 mL), was added oxalyl chloride (1.422 mL, 16.25 mmol) followed by a few drops of DMF at room temperature. The reaction was stirred for 1 h at room temperature. The reaction was concentrated under reduced pressure and the resulting residue was dissolved in CH₂Cl₂ (5 mL), and then 4-chloro-2-(4-(3,3,3-trifluoropropyl)piperazin-1-yl)aniline (1.0 g, 3.25 mmol) and DIEA (0.420 g, 3.25 mmol) were added. The reaction was stirred for 30 min at room temperature. The reaction was washed with water and dried over MgSO₄. The organic layer was concentrated under reduced pressure and the residue was crystallized in methanol. The precipitate was filtered and dried under reduced pressure to give the title compound (1.45 g, 85%) as a mixture of bromo- and chloro-intermediates. LC/MS (peak 1) m/z=478.0 [M+H]⁺, (peak 2) m/z=523.2 [M+H]⁺.

### Step C: Preparation of N-(4-Chloro-2-(4-(3,3,3-trifluoropropyl)piperazin-1-yl)phenyl)-4-((cyanomethylamino)methyl)-2-fluorobenzamide (Compound 65)

Intermediate 2 (10 mg, 0.019 mmol, Step B), 2-aminoacetonitrile (3.5 mg, 0.023 mmol), and DIEA (10.02 μL, 0.057 mmol) were added to a vial with DMF (0.2 mL). The reaction was heated to 80° C. and stirred at this temperature for 30 min. The mixture was purified by preparative LC/MS to give the title compound (5.0 mg, 33.8%). LC/MS m/z=498.4 [M+H]⁺.

### Examples 1.22 to 1.31

The following compounds were prepared using the disclosed intermediates and a method similar to the one described in Example 1.21, Step C.

| | | | Product |
|---|---|---|---|
| Example | Cmpd | | (Mass Observed |
| No. | No. | Intermediates Used | LC/MS m/z) |
| 22 | 66¹ | Intermediate 2 and 1-(2- | 638.6 [M + H]⁺ |
| | | cyclohexylethyl)piperazine | |
| 23 | 67 | Intermediate 2 and (S)-2-aminopropanamide | 530.4 [M + H]⁺ |
| 24 | 68² | Intermediate 2 and tetrahydrothiophen-3-amine- | 577.4 [M + H]⁺ |
| | | 1,1-dioxide | |
| 25 | 69³ | Intermediate 2 and (R)-2-amino-4-methylpentan- | 559.2 [M + H]⁺ |
| | | 1-ol | |
| 26 | 70 | Intermediate 2 and 2-(1H-imidazol-5-yl)-N- | 567.4 [M + H]⁺ |
| | | methylethanamine dihydrochloride | |
| 27 | 71 | Intermediate 2 and (1-methyl-1H-imidazol-5- | 553.8 [M + H]⁺ |
| | | yl)methanamine | |
| 28 | 72 | Intermediate 2 and 2,7-diazaspiro[4.4] nonane | 568.2 [M + H]⁺ |
| | | dihydrochloride | |
| 29 | 73 | Intermediate 2 and (1R,2S)-2- | 584.2 [M + H]⁺ |
| | | aminocyclohexanecarboxamide | |
| 30 | 74 | Intermediate 2 and (S)-methyl 2-amino-3- | 560.8 [M + H]⁺ |
| | | hydroxypropanoate hydrochloride | |
| 31 | 75 | Intermediate 2 and 2-aminopropane-1,3-diol | 533.2 [M + H]⁺ |

¹Compound 66: ¹H NMR (400 MHz, DMSO-d₆) δ ppm 0.84-0.98 (m, 2H), 1.05-1.33 (m, 5H), 1.48-1.57 (m, 2H), 1.57-1.73 (m, 5H), 2.80-3.06 (m, 7H), 3.06-3.14 (m, 3H), 3.19 (bs, 5H), 3.34-3.60 (m, 6H), 3.74 (s, 2H), 7.29 (dd, J = 8.78, 2.34 Hz, 1H), 7.34-7.40 (m, 3H), 7.91 (t, J = 7.89 Hz, 1H), 8.28 (d, J = 8.59 Hz, 1H), 9.69 (d, J = 8.34 Hz, 1H).
²Compound 68: ¹H NMR (400 MHz, DMSO-d₆) δ ppm 2.17-2.30 (m, 1H), 2.52-2.63 (m, 1H), 2.70-2.87 (m, 3H), 2.95-3.73 (m, 14H), 4.03 (bs, 1H), 4.25 (s, 2H), 7.28 (dd, J = 8.78, 2.21 Hz, 1H), 7.38 (d, J = 2.02 Hz, 1H), 7.50 (d, J = 8.21 Hz, 1H), 7.55 (d, J = 11.87 Hz, 1H), 7.99 (t, J = 7.77 Hz, 1H), 8.26 (d, J = 8.59 Hz, 1H), 9.74 (d, J = 8.21 Hz, 1H).
³Compound 69: ¹H NMR (400 MHz, DMSO-d₆) δ ppm 0.84 (d, J = 6.32 Hz, 3H), 0.91 (d, J = 6.44 Hz, 3H), 1.41-1.51 (m, 1H), 1.54-1.74 (m, 2H), 2.70-2.90 (m, 2H), 3.03-3.32 (m, 12H), 3.76 (dd, J = 12.32, 3.09 Hz, 2H), 4.31 (s, 2H), 7.28 (dd, J = 8.72, 2.27 Hz, 1H), 7.38 (d, J = 1.77 Hz, 1H), 7.53 (d, J = 8.08 Hz, 1H), 7.59 (d, J = 12.50 Hz, 1H), 7.98 (t, J = 7.96 Hz, 1H), 8.25 (d, J = 8.21 Hz, 1H), 8.80-8.99 (m, 2H), 9.74 (d, J = 7.96 Hz, 1H).

### Example 1.32

### Preparation of (S)-4-(1-Amino-3-hydroxy-1-oxopropan-2-ylamino)methyl)-N-(4-chloro-2-(4-(3,3,3-trifluoropropyl)piperazin-1-yl)phenyl)-2-fluorobenzamide (Compound 76)

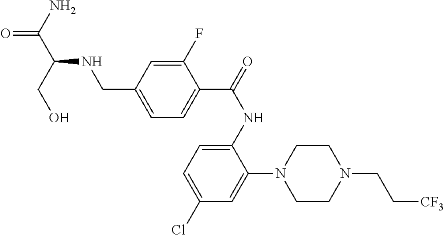 [SMILES: NC(=O)[C@H](CO)NCc1ccc(C(=O)Nc2ccc(Cl)cc2N2CCN(CCC(F)(F)F)CC2)c(F)c1]

From Intermediate 2 and (S)-2-amino-3-hydroxypropanamide hydrochloride, using a similar method to the one described in Example 1.21, Step C, the title compound was obtained. LC/MS m/z=546.2 [M+H]⁺.

### Examples 1.33 to 1.105

The following compounds were prepared using the disclosed intermediates and a method similar to the one described in Example 1.21, Step C.

| | | | Product |
|---|---|---|---|
| Example | Cmpd | | (Mass Observed |
| No. | No. | Intermediates Used | LC/MS m/z) |
| 1.33 | 77 | Intermediate 2 and 3- | 591.4 [M + H]⁺ |
| | | (methylsulfonyl)pyrrolidine | |
| 1.34 | 78 | Intermediate 2 and (1S,2S)-2- | 557.4 [M + H]⁺ |
| | | aminocyclohexanol hydrochloride | |
| 1.35 | 79 | Intermediate 2 and 3-aminoazepan-2-one | 570.4 [M + H]⁺ |
| 1.36 | 80 | Intermediate 2 and 2-(isopropylamino)ethanol | 545.4 [M + H]⁺ |
| 1.37 | 81 | Intermediate 2 and 1-(2- | 571.4 [M + H]⁺ |
| | | aminoethyl)imidazolidin-2-one | |
| 1.38 | 82 | Intermediate 2 and 4,5,6,7-tetrahydro-1H- | 565.2 [M + H]⁺ |
| | | imidazo[4,5-c]pyridine dihydrochloride | |
| 1.39 | 83¹ | Intermediate 2 and piperidin-2-ylmethanol | 557.4 [M + H]⁺ |
| 1.40 | 94 | Intermediate 2 and piperidine-2-carboxamide | 570.4 [M + H]⁺ |
| 1.41 | 95 | Intermediate 2 and 1,4,5,6- | 526.6 [M + H]⁺ |
| | | tetrahydropyrimidine | |
| 1.42 | 96 | Intermediate 2 and (4R)-methyl 4- | 587.4 [M + H]⁺ |
| | | hydroxypyrrolidine-2-carboxylate hydrochloride | |
| 1.43 | 97 | Intermediate 2 and 1-aminopropan-2-ol | 517.4 [M + H]⁺ |
| 1.44 | 98 | Intermediate 2 and piperidin-4-ol | 543.2 [M + H]⁺ |
| 1.45 | 99 | Intermediate 2 and 1-(2-(pyridin-2- | 633.6 [M + H]⁺ |
| | | yl)ethyl)piperazine | |
| 1.46 | 100 | Intermediate 2 and 3-(ethylamino)pyridin-2-ol | 580.4 [M + H]⁺ |
| | | dihydrochloride | |
| 1.47 | 101 | Intermediate 2 and 2-(4-methylpiperazin-1- | 585.4 [M + H]⁺ |
| | | yl)ethanamine | |
| 1.48 | 102 | Intermediate 2 and (S)-2-aminopropan-1-ol | 517.4 [M + H]⁺ |
| 1.49 | 103 | Intermediate 2 and 2-amino-2- | 563.4 [M + H]⁺ |
| | | (hydroxymethyl)propane-1,3-diol | |
| 1.50 | 104 | Intermediate 2 and piperidin-3-ol | 543.6 [M + H]⁺ |
| 1.51 | 105 | Intermediate 2 and N-(2-aminoethyl)acetamide | 544.4 [M + H]⁺ |
| 1.52 | 106 | Intermediate 2 and (R)-2-aminobutan-1-ol | 531.4 [M + H]⁺ |
| 1.53 | 107 | Intermediate 2 and N,N-diethylpiperidine-3- | 626.4 [M + H]⁺ |
| | | carboxamide | |
| 1.54 | 108 | Intermediate 2 and 2,3,4,6,7,8-hexahydro-1H- | 581.8 [M + H]⁺ |
| | | pyrimido[1,2-a]pyrimidine | |
| 1.55 | 109 | Intermediate 2 and 2-(1-methylpyrrolidin-2- | 570.0 [M + H]⁺ |
| | | yl)ethanamine | |
| 1.56 | 110 | Intermediate 2 and N¹,N¹,N²-trimethylethane-1, | 544.6 [M + H]⁺ |
| | | 2-diamine | |
| 1.57 | 159 | Intermediate 2 and 2-(pyrrolidin-1- | 556.2 [M + H]⁺ |
| | | yl)ethanamine | |
| 1.58 | 111 | Intermediate 2 and 1-cyclopentylpiperazine | 596.6 [M + H]⁺ |
| 1.59 | 112 | Intermediate 2 and 1,4-oxazepane | 543.4 [M + H]⁺ |
| | | hydrochloride | |
| 1.60 | 113 | Intermediate 2 and (S)-1,2′- | 596.6 [M + H]⁺ |
| | | methylenedipyrrolidine | |
| 1.61 | 114 | Intermediate 2 and 2-morpholinoethanamine | 572.2 [M + H]⁺ |
| 1.62 | 115 | Intermediate 2 and 4-(piperidin-4- | 612.4 [M + H]⁺ |
| | | yl)morpholine | |
| 1.63 | 116 | Intermediate 2 and N¹,N¹-dimethylethane-1,2- | 530.6 [M + H]⁺ |
| | | diamine | |
| 1.64 | 117 | Intermediate 2 and 3-amino-1-ethylazepan-2- | 598.4 [M + H]⁺ |
| | | one | |
| 1.65 | 118 | Intermediate 2 and 1- | 624.2 [M + H]⁺ |
| | | (cyclohexylmethyl)piperazine | |
| 1.66 | 119 | Intermediate 2 and piperidin-4-one | 541.2 [M + H]⁺ |
| | | hydrochloride hydrate | |
| 1.67 | 120 | Intermediate 2 and 3-(aminomethyl)-N,N- | 602.6 [M + H]⁺ |
| | | dimethyltetrahydrothiophen-3-amine | |
| 1.68 | 121 | Intermediate 2 and N¹,N¹-diethyl-N²- | 572.2 [M + H]⁺ |
| | | methylethane-1,2-diamine | |
| 1.69 | 122 | Intermediate 2 and (2S,3S)-2-amino-3- | 559.4 [M + H]⁺ |
| | | methylpentan-1-ol | |
| 1.70 | 123 | Intermediate 2 and pentane-1,5-diamine | 544.4 [M + H]⁺ |
| 1.71 | 124 | Intermediate 2 and 3-amino-3- | 543.4 [M + H]⁺ |
| | | iminopropanamide hydrochloride | |
| 1.72 | 125 | Intermediate 2 and 1-(piperazin-1-yl)ethanone | 570.6 [M + H]⁺ |
| 1.73 | 126 | Intermediate 2 and 1,4′-bipiperidine | 610.6 [M + H]⁺ |
| 1.74 | 127 | Intermediate 2 and 1- | 571.4 [M + H]⁺ |
| | | (aminomethyl)cyclohexanol hydrochloride | |
| 1.75 | 128 | Intermediate 2 and (S)-3-aminoazepan-2-one | 570.4 [M + H]⁺ |
| 1.76 | 129 | Intermediate 2 and ethyl piperidine-4- | 599.6 [M + H]⁺ |
| | | carboxylate | |
| 1.77 | 130 | Intermediate 2 and (R)-1-aminopropan-2-ol | 517.4 [M + H]⁺ |
| 1.78 | 131 | Intermediate 2 and morpholin-2-ylmethanol | 559.4 [M + H]⁺ |
| 1.79 | 132 | Intermediate 2 and (R)-(1-(2- | 586.4 [M + H]⁺ |
| | | aminoethyl)pyrrolidin-2-yl)methanol | |
| 1.80 | 133 | Intermediate 2 and 2-methyl-2-(piperidin-1- | 598.6 [M + H]⁺ |
| | | yl)propan-1-amine | |
| 1.81 | 134 | Intermediate 2 and (S)-pyrrolidin-2-ylmethanol | 543.4 [M + H]⁺ |
| 1.82 | 135 | Intermediate 2 and azetidin-3-ol hydrochloride | 515.4 [M + H]⁺ |
| 1.83 | 136 | Intermediate 2 and (S)-1-aminopropan-2-ol | 517.4 [M + H]⁺ |
| 1.84 | 137 | Intermediate 2 and 1,4′-bipiperidin-4-ol | 626.6 [M + H]⁺ |
| 1.85 | 138 | Intermediate 2 and N¹,N¹-diethylethane-1,2- | 558.4 [M + H]⁺ |
| | | diamine | |
| 1.86 | 139 | Intermediate 2 and 2-(benzylamino)ethanol | 593.4 [M + H]⁺ |
| 1.87 | 140 | Intermediate 2 and 2- | 549.4 [M + H]⁺ |
| | | (methylsulfinyl)ethanamine | |
| 1.88 | 141 | Intermediate 2 and pyrrolidin-3-ol | 529.4 [M + H]⁺ |
| 1.89 | 142 | Intermediate 2 and 3,3′- | 565.0 [M + H]⁺ |
| | | azanediyldipropanenitrile | |
| 1.90 | 143 | Intermediate 2 and 2-(azepan-1-yl)ethanamine | 584.6 [M + H]⁺ |
| 1.91 | 144 | Intermediate 2 and 2,2′-azanediyldiethanol | 547.4 [M + H]⁺ |
| 1.92 | 145 | Intermediate 2 and 4-aminobutan-2-ol | 531.4 [M + H]⁺ |
| 1.93 | 146 | Intermediate 2 and 2-(tert-butylamino)ethanol | 559.4 [M + H]⁺ |
| 1.94 | 147 | Intermediate 2 and piperidin-3-ylmethanol | 557.6 [M + H]⁺ |
| 1.95 | 148 | Intermediate 2 and 2,5- | 540.6 [M + H]⁺ |
| | | diazabicyclo[2.2.1]heptane dihydrobromide | |
| 1.96 | 149 | Intermediate 2 and (S)-2-amino-3- | 558.4 [M + H]⁺ |
| | | methylbutanamide | |
| 1.97 | 150 | Intermediate 2 and 2,2′-(ethane-1,2- | 590.4 [M + H]⁺ |
| | | diylbis(oxy))diethanamine | |
| 1.98 | 151 | Intermediate 2 and (S)-(1-(2- | 586.2 [M + H]⁺ |
| | | aminoethyl)pyrrolidin-2-yl)methanol | |
| 1.99 | 152 | Intermediate 2 and (R)-methyl 2-amino-3- | 561.4 [M + H]⁺ |
| | | hydroxypropanoate hydrochloride | |
| 1.100 | 153 | Intermediate 2 and (2R,3S)-2-aminobutane-1,3- | 547.4 [M + H]⁺ |
| | | diol hydrochloride | |
| 1.101 | 154 | Intermediate 2 and 2-amino-1- | 586.4 [M + H]⁺ |
| | | morpholinoethanone hydrochloride | |
| 1.102 | 155 | Intermediate 2 and N¹,N¹-dimethyl-1-(pyridin- | 607.6 [M + H]⁺ |
| | | 3-yl)ethane-1,2-diamine | |
| 1.103 | 156 | Intermediate 2 and 1,4-diazepan-5-one | 556.6 [M + H]⁺ |
| 1.104 | 157 | Intermediate 2 and octahydropyrrolo[1,2- | 568.6 [M + H]⁺ |
| | | a]pyrazine | |
| 1.105 | 158 | Intermediate 2 and 2-(methylamino)-1- | 600.0 [M + H]⁺ |
| | | morpholinoethanone dihydrochloride | |

¹Compound 83: ¹H NMR (400 MHz, DMSO-d₆) δ ppm 1.35-2.02 (m, 6H), 2.78-3.05 (m, 3H), 3.07-3.24 (m, 7H), 3.28-3.46 (m, 6H), 4.03 (dd, J = 12.57, 3.35 Hz, 2H), 4.24 (d, J = 13.64 Hz, 1H), 4.75 (d, J = 13.14 Hz, 1H), 7.29 (dd, J = 8.72, 2.27 Hz, 1H), 7.37 (d, J = 1.89 Hz, 1H), 7.53 (d, J = 8.08 Hz, 1H), 7.58 (d, J = 11.49 Hz, 1H), 7.98 (t, J = 7.89 Hz, 1H), 8.23 (d, J = 8.59 Hz, 1H), 9.42 (bs, 1H), 9.76 (d, J = 7.07 Hz, 1H).

### Example 1.106

### Preparation of (S)-4-(1-Amino-3-hydroxy-1-oxopropan-2-ylamino)methyl)-N-(4,5-dichloro-2-(4-(3,3,3-trifluoropropyl)piperazin-1-yl)phenyl)-2-fluorobenzamide (Compound 160)

### Step A: Preparation of 1-(4,5-Dichloro-2-nitrophenyl)piperazine

Piperazine (6.15 g, 71.4 mmol) was dissolved in IPA (50 mL) and cooled in an ice bath. 1,2-dichloro-4-fluoro-5-nitrobenzene (5 g, 23.81 mmol), pre-dissolved in IPA (10 mL), was added to the reaction slowly via addition funnel (the solution turned yellowish orange over time). Upon complete addition, the reaction was warmed in an oil bath to 80° C. and stirred at this temperature for 1 h (the solution turned reddish-orange). After this time, the reaction was complete. The solvent was evaporated and an extraction was performed (100 mL each of H₂O and EtOAc). A tiny amount of insoluble precipitate was filtered off during the extraction. The aqueous layer was extracted twice more with EtOAc (100 mL). The organic layers were combined and back extracted once with H₂O/brine (250 mL). The organic layer was dried and concentrated to give the title compound (7.86 g, 96%), as a reddish-brown oil. LC/MS m/z=276.0 [M+H]⁺; ¹H NMR (400 MHz, DMSO-d₆) δ ppm 2.74-2.79 (m, 4H), 2.92-2.95 (m, 4H), 7.50 (s, HD, 8.12 (s, HD.

### Step B: Preparation of 4,5-Dichloro-2-(4-(3,3,3-trifluoropropyl)piperazin-1-yl)aniline

1-(4,5-Dichloro-2-nitrophenyl)piperazine (6.57 g, 23.79 mmol) was dissolved in THF (40 mL) and MeOH (10 mL). DIEA (8.31 mL, 47.6 mmol) and 3-bromo-1,1,1-trifluoropropane (3.82 mL, 35.7 mmol) were added and the reaction was heated to reflux in an oil bath over night. The next day, the reaction was approximately 40% complete. Thus, more 3-bromo-1,1,1-trifluoropropane (3.82 mL, 35.7 mmol) was added. The reaction was continued to stir at reflux over the weekend. After this time, the starting material had been consumed. The solvent was evaporated to give the crude intermediate, 1-(4,5-dichloro-2-nitrophenyl)-4-(3,3,3-trifluoropropyl)piperazine. This crude material was re-dissolved in EtOH (20 mL) and cooled on ice. To the stirring solution was added tin(II) chloride (13.5 g, 71.4 mmol) portionwise (slight exotherm). After complete addition, the reaction was heated in an oil bath to 80° C. for 1 h. The reaction was cooled on an ice bath. Then, 50% w/v conc. NaOH (˜30 mL), some H₂O (120 mL), and CH₂Cl₂ (150 mL) were added to the reaction until it was completely quenched and the tin precipitate was re-dissolved. The reaction was extracted twice. The organic layer was dried, concentrated, and the residue was purified by column chromatography (0-30% EtOAc/hexanes) to give the title compound (7.71 g, 92%) as a red oil. LC/MS m/z=342.2 [M+H]⁺; ¹H NMR (400 MHz, DMSO-d₆) δ ppm 2.43-2.48 (m, 2H), 2.53-2.61 (m, 6H), 2.76-2.82 (m, 4H), 5.10 (s, 2H), 6.84 (s, 1H), 6.99 (s, 1H).

### Step C: Preparation of 4-(Bromomethyl)-N-(4,5-dichloro-2-(4-(3,3,3-trifluoropropyl)piperazin-1-yl)phenyl)-2-fluorobenzamide

4-(Bromomethyl)-2-fluorobenzoic acid (0.681 g, 2.92 mmol) was suspended in CH₂Cl₂ (10 mL). DIEA (0.766 mL, 4.38 mmol) was added (the starting material dissolved), and the reaction was cooled on an ice bath. Thionyl chloride (0.640 mL, 8.77 mmol) was added (the reaction fumed and turned dark brown). The reaction was warmed to room temperature and stirred for 1 h. After this time, the solvent was completely evaporated. The resulting brown oil was re-dissolved in CH₂Cl₂ (20 mL) and cooled on an ice bath again. Another aliquot of DIEA (0.766 mL, 4.38 mmol) was added, followed by 4,5-dichloro-2-(4-(3,3,3-trifluoropropyl)piperazin-1-yl)aniline (1.0 g, 2.92 mmol). The reaction was warmed to room temperature and stirred for 1 h. After this time, the starting material was consumed. The reaction was extracted (2×50 mL of NaHCO₃/H₂O and CH₂Cl₂). The organic layers were combined, dried, concentrated, and the residue was purified by column chromatography (0-30% EtOAc/hexanes) to give the title compound (1.17 g, 70.4%) as a brown solid. LC/MS m/z=512.2 [M+H]⁺; ¹H NMR (400 MHz, DMSO-d₆) δ ppm 2.44-2.68 (m, 8H), 2.89 (t, T=4.55 Hz, 4H), 4.85 (s, 2H), 7.48 (dd, T=8.08, 1.64 Hz, 1H), 7.52-7.61 (m, 2H), 8.00 (t, T=8.08 Hz, 1H), 8.57 (s, 1H), 9.84 (d, T=10.74 Hz, 1H).

### Step D: Preparation of (S)-4-((1-Amino-3-hydroxy-1-oxopropan-2-ylamino)methyl)-N-(4,5-dichloro-2-(4-(3,3,3-trifluoropropyl)piperazin-1-yl)phenyl)-2-fluorobenzamide (Compound 160) as the Bis(2,2,2-trifluoroacetate) Salt

4-(Bromomethyl)-N-(4,5-dichloro-2-(4-(3,3,3-trifluoropropyl)piperazin-1-yl)phenyl)-2-fluorobenzamide (10 mg, 0.018 mmol), (S)-2-amino-3-hydroxypropanamide hydrochloride (3.03 mg, 0.022 mmol), and DIEA (31.3 μL, 0.179 mmol) were added to a vial with DMF (0.2 mL). The reaction was heated to 80° C. and stirred at this temperature for 30 min. The mixture was purified by preparative LC/MS (5-70% ACN/H₂O, 25 min) to give the title compound. LC/MS m/z=580.6 [M+H]⁺; ¹H NMR (400 MHz, DMSO-d₆) δ ppm 2.78-2.93 (m, 2H), 3.17 (s, 5H), 3.27-3.46 (m, 6H), 3.82-3.91 (m, 5H), 4.27 (s, 2H), 7.51 (dd, J=8.08, 1.39 Hz, 1H), 7.54-7.61 (m, 2H), 7.69 (s, 1H), 7.90 (s, 1H), 7.96 (t, J=7.96 Hz, 1H), 8.51 (s, 1H), 9.35 (bs, 2H), 9.82 (d, J=7.58 Hz, 1H).

### Examples 1.107 to 1.110

The following compounds were prepared using the disclosed intermediates and a method similar to the one described in Example 1.106, Step D.

| | | | Product |
|---|---|---|---|
| Example | Cmpd | | (Mass Observed |
| No. | No. | Intermediates Used | LC/MS m/z) |
| 1.107 | 161¹ | 4-(bromomethyl)-N-(4,5-dichloro-2-(4-(3,3,3- | 611.4 [M + H]⁺ |
| | | trifluoropropyl)piperazin-1-yl)phenyl)-2- | |
| | | fluorobenzamide and thiomorpholin-1,1-dioxide | |
| 1.108 | 162² | 4-(bromomethyl)-N-(4,5-dichloro-2-(4-(3,3,3- | 590.4 [M + H]⁺ |
| | | trifluoropropyl)piperazin-1-yl)phenyl)-2- | |
| | | fluorobenzamide and (S)-pyrrolidine-2- | |
| | | carboxamide | |
| 1.109 | 163³ | 4-(bromomethyl)-N-(4,5-dichloro-2-(4-(3,3,3- | 576.4 [M + H]⁺ |
| | | trifluoropropyl)piperazin-1-yl)phenyl)-2- | |
| | | fluorobenzamide and piperazin-2-one | |
| 1.110 | 164⁴ | 4-(bromomethyl)-N-(4,5-dichloro-2-(4-(3,3,3- | 611.4 [M + H]⁺ |
| | | trifluoropropyl)piperazin-1-yl)phenyl)-2- | |
| | | fluorobenzamide and tetrahydrothiophen-3- | |
| | | amine-1,1-dioxide | |

¹Compound 161: ¹H NMR (400 MHz, DMSO-d₆) δ ppm 2.79-2.98 (m, 7H), 3.07-3.25 (m, 8H), 3.28-3.48 (m, 5H), 3.82 (s, 2H), 7.37 (d, J = 2.02 Hz, 1H), 7.40 (s, 1H), 7.59 (s, 1H), 7.91 (t, J = 7.89 Hz, 1H), 8.55 (s, 1H), 9.76 (d, J = 8.46 Hz, 1H).
²Compound 162: ¹H NMR (400 MHz, DMSO-d₆) δ ppm 1.81-1.97 (m, 2H), 1.98-2.15 (m, 1H), 2.75-2.88 (m, 3H), 3.02-3.32 (m, 12H), 4.10 (bs, 2H), 4.34-4.54 (m, 3H), 7.49-7.62 (m, 4H), 7.66 (bs, 1H), 7.93 (bs, 1H), 7.98 (t, J = 7.89 Hz, 1H), 8.51 (s, 1H), 9.84 (d, J = 7.96 Hz, 1H).
³Compound 163: ¹H NMR (400 MHz, DMSO-d₆) δ ppm 2.77-2.98 (m, 5H), 3.09-3.51 (m, 13H), 3.95 (s, 2H), 7.40-7.47 (m, 2H), 7.59 (s, 1H), 7.94 (t, J = 8.02 Hz, 1H), 8.01 (bs, 1H), 8.54 (s, 1H), 9.80 (d, J = 8.21 Hz, 1H).
⁴Compound 164: ¹H NMR (400 MHz, DMSO-d₆) δ ppm 2.18-2.31 (m, 1H), 2.53-2.64 (m, 1H), 2.74-2.88 (m, 3H), 3.02-3.70 (m, 14H), 4.04 (bs, 1H), 4.27 (s, 2H), 7.49-7.61 (m, 3H), 7.99 (t, J = 7.96 Hz, 1H), 8.52 (s, 1H), 9.83 (d, J = 7.96 Hz, 1H).

### Example 1.111

### Preparation of (S)-4-(1-Amino-3-hydroxy-1-oxopropan-2-ylamino)methyl)-N-(4,5-dichloro-2-(4-(3,3,3-trifluoropropyl)piperazin-1-yl)phenyl)-2,3-difluorobenzamide (Compound 185)

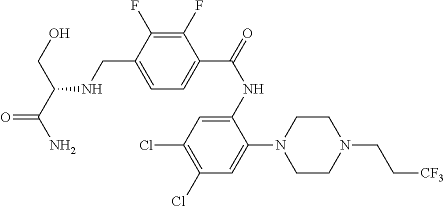 [SMILES: NC(=O)[C@H](CO)NCc1ccc(C(=O)Nc2cc(Cl)c(Cl)cc2N2CCN(CCC(F)(F)F)CC2)c(F)c1F]

### Step A: Preparation of 4-(Bromomethyl)-N-(4,5-dichloro-2-(4-(3,3,3-trifluoropropyl)piperazin-1-yl)phenyl)-2,3-difluorobenzamide

4-(Bromomethyl)-2,3-difluorobenzoic acid (575 mg, 2.291 mmol) and DIEA (600 μL, 3.44 mmol) were dissolved in CH₂Cl₂ (10 mL). The solution was cooled on an ice bath, and then thionyl chloride (502 μL, 6.87 mmol) was added slowly (the solution turned dark). The reaction was warmed to room temperature and stirred at this temperature for 1 h. After this time, the solvent was completely evaporated. The resulting dark purple oil was re-dissolved in CH₂Cl₂ (10 mL), and cooled on an ice bath. Another aliquot of DIEA (600 μL, 3.44 mmol) was added, followed by 4,5-dichloro-2-(4-(3,3,3-trifluoropropyl)piperazin-1-yl)aniline (784 mg, 2.291 mmol). The reaction was warmed to room temperature and stirred for 1 h. The reaction mixture was purified by column chromatography (0-30% EtOAc/hexanes) to give the title compound (319 mg, 21.79%), as a light reddish-brown solid. LC/MS m/z=532.2 [M+H]⁺; ¹H NMR (400 MHz, DMSO-d₆) δ ppm 2.44-2.65 (m, 8H), 2.90 (t, J=4.48 Hz, 4H), 4.91 (s, 2H), 7.51-7.58 (m, 2H), 7.69-7.76 (m, 1H), 8.45 (s, 1H), 9.85 (d, J=7.07 Hz, 1H).

### Step B: Preparation of (S)-4-(1-Amino-3-hydroxy-1-oxopropan-2-ylamino)methyl)-N-(4,5-dichloro-2-(4-(3,3,3-trifluoropropyl)piperazin-1-yl)phenyl)-2,3-difluorobenzamide (Compound 185) as the bis(2,2,2-trifluoroacetate) salt

4-(Bromomethyl)-N-(4,5-dichloro-2-(4-(3,3,3-trifluoropropyl)piperazin-1-yl)phenyl)-2,3-difluorobenzamide (10 mg, 0.017 mmol), (S)-2-amino-3-hydroxypropanamide hydrochloride (2.9 mg, 0.021 mmol), and DIEA (30.4 μL, 0.174 mmol) were added to a vial with DMF (0.2 mL). The reaction was heated to ˜80° C. and stirred at this temperature for 30 min. The reaction mixture was purified by preparative LC/MS (5-70% ACN/H₂O (0.1% TFA), 25 min) to give the title compound. LC/MS m/z=598.4 [M+H]⁺.

### Examples 1.112, 1.113, and 1.115 to 1.118

The following compounds were prepared using the disclosed intermediates and a method similar to the one described in Example 1.111, Step B.

| | | | Product |
|---|---|---|---|
| Example | Cmpd | | (Mass Observed |
| No. | No. | Intermediates Used | LC/MS m/z) |
| 1.112 | 186 | 4-(bromomethyl)-N-(4,5-dichloro-2-(4-(3,3,3- | 629.4 [M + H]⁺ |
| | | trifluoropropyl)piperazin-1-yl)phenyl)-2,3- | |
| | | difluorobenzamide and thiomorpholin-1,1- | |
| | | dioxide | |
| 1.113 | 187¹ | 4-(bromomethyl)-N-(4,5-dichloro-2-(4-(3,3,3- | 608.6 [M + H]⁺ |
| | | trifluoropropyl)piperazin-1-yl)phenyl)-2,3- | |
| | | difluorobenzamide and (S)-pyrrolidine-2- | |
| | | carboxamide | |
| 1.115 | 189 | 4-(bromomethyl)-N-(4,5-dichloro-2-(4-(3,3,3- | 629.2 [M + H]⁺ |
| | | trifluoropropyl)piperazin-1-yl)phenyl)-2,3- | |
| | | difluorobenzamide and tetrahydrothiophen-3- | |
| | | amine-1,1-dioxide | |
| 1.116 | 190² | 4-(bromomethyl)-N-(4,5-dichloro-2-(4-(3,3,3- | 623.6 [M + H]⁺ |
| | | trifluoropropyl)piperazin-1-yl)phenyl)-2,3- | |
| | | difluorobenzamide and 2-(piperidin-4-yl)ethanol | |
| 1.117 | 191 | 4-(bromomethyl)-N-(4,5-dichloro-2-(4-(3,3,3- | 611.4 [M + H]⁺ |
| | | trifluoropropyl)piperazin-1-yl)phenyl)-2,3- | |
| | | difluorobenzamide and morpholin-2-ylmethanol | |
| 1.118 | 192³ | 4-(bromomethyl)-N-(4,5-dichloro-2-(4-(3,3,3- | 567.2 [M + H]⁺ |
| | | trifluoropropyl)piperazin-1-yl)phenyl)-2,3- | |
| | | difluorobenzamide and azetidin-3-ol | |

¹Compound 187: ¹H NMR (400 MHz, DMSO-d₆) δ ppm 1.78-1.94 (m, 2H), 1.96-2.12 (m, 1H), 2.39-2.48 (m, 1H), 2.74-2.92 (m, 3H), 3.06-3.46 (m, 10H), 4.04 (bs, 2H), 4.31-4.58 (m, 3H), 7.50-7.60 (m, 2H), 7.63-7.76 (m, 2H), 7.95 (bs, 1H), 8.41 (s, 1H), 9.92 (d, J = 3.92 Hz, 1H).
²Compound 190: ¹H NMR (400 MHz, DMSO-d₆) δ ppm 1.24-1.43 (m, 4H), 1.56-1.75 (m, 2H), 1.81-1.96 (m, 2H), 2.73-2.90 (m, 2H), 3.08-3.69 (m, 16H), 4.45 (s, 2H), 7.51-7.61 (m, 2H), 7.75 (t, J = 6.95 Hz, 1H), 8.42 (s, 1H), 9.80 (bs, 1H), 9.93 (d, J = 4.29 Hz, 1H).
³Compound 192: ¹H NMR (400 MHz, DMSO-d₆) δ ppm 2.72-2.91 (m, 2H), 3.04-3.74 (m, 11H), 3.92-3.99 (m, 2H), 4.29-4.36 (m, 2H), 4.41-4.55 (m, 1H), 4.59 (s, 2H), 7.51 (t, J = 7.01 Hz, 1H), 7.57 (s, 1H), 7.73 (t, J = 6.88 Hz, 1H), 8.41 (s, 1H), 9.94 (d, J = 4.29 Hz, 1H), 10.22-11.03 (m, 1H).

### Example 1.114

### Preparation of N-(4,5-Dichloro-2-(4-(3,3,3-trifluoropropyl)piperazin-1-yl)phenyl)-2,3-difluoro-4-((3-oxopiperazin-1-yl)methyl)benzamide (Compound 188)

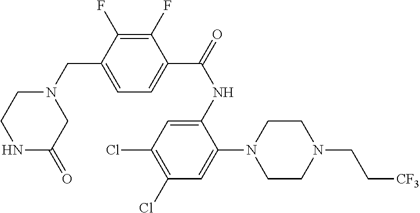 [SMILES: O=C1CN(Cc2ccc(C(=O)Nc3cc(Cl)c(Cl)cc3N3CCN(CCC(F)(F)F)CC3)c(F)c2F)CCN1]

From 4-(bromomethyl)-N-(4,5-dichloro-2-(4-(3,3,3-trifluoropropyl)piperazin-1-yl)phenyl)-2,3-difluorobenzamide and piperazin-2-one, using a similar method to the one described in Example 1.111, Step B, the title compound was obtained. LC/MS m/z=594.4 [M+H]⁺; ¹H NMR (400 MHz, DMSO-d₆) δ ppm 2.75-2.97 (m, 4H), 3.00-3.55 (m, 12H), 3.89 (s, 4H), 7.45 (t, J=6.95 Hz, 1H), 7.58 (s, 1H), 7.67 (t, 1H), 7.91 (s, 1H), 8.45 (s, 1H), 9.84 (d, J=4.67 Hz, 1H).

### Example 1.119

### Preparation of (R)-Methyl 3-amino-2-(4-(4-chloro-2-(4-(3,3,3-trifluoropropyl)piperazin-1-yl)phenylcarbamoyl)-3-fluorobenzylamino)propanoate (Compound 196)

Intermediate 2 (10 mg, 0.019 mmol), (R)-methyl 2-amino-3-(tert-butoxycarbonylamino)propanoate (4.17 mg, 0.019 mmol), and DIEA (3.34 μL, 0.019 mmol) were added to a vial with DMF (0.4 mL). The reaction was heated to 80° C. and stirred at this temperature for 1 h. After this time, the reaction was complete. The N-Boc protected intermediate was purified by preparative LC/MS (5-70% ACN/H₂O, 25 min). After lyophilization, the solid was re-dissolved in MeOH (0.4 mL) and TFA (0.1 mL) (to cleave the N-Boc group). The reaction was allowed to stir at room temperature over night. Then, the solvent was removed, the oil was re-dissolved in ACN (0.4 mL) and H₂O (0.8 mL), frozen, and lyophilized to give the title compound. LC/MS m/z=560.4 [M+H]⁺; ¹H NMR (400 MHz, DMSO-d₆) δ ppm 1.21-1.31 (m, 1H), 2.75-2.91 (m, 3H), 3.05-3.20 (m, 2H), 3.33-3.81 (m, 17H), 7.29 (dd, J=8.84, 2.27 Hz, 1H), 7.36-7.43 (m, 2H), 7.47 (d, J=13.14 Hz, 1H), 7.89 (t, J=8.02 Hz, 1H), 8.30 (d, J=8.08 Hz, 1H), 9.65 (d, J=8.97 Hz, 1H).

### Examples 1.120 and 1.121

The following compounds were prepared using the disclosed intermediates and a method similar to the one described in Example 1.119.

| | | | Product |
|---|---|---|---|
| | | | (Mass |
| Example | Cmpd | | Observed |
| No. | No. | Intermediates Used | LC/MS m/z) |
| 1.120 | 197¹ | Intermediate 2 and (R)-methyl 2- | 574.4 |
| | | amino-4-(tert-butoxycarbonylamino)- | [M + H]⁺ |
| | | butanoate | |
| 1.121 | 198 | Intermediate 2 and (S)-methyl 2- | 560.2 |
| | | amino-3-(tert butoxycarbonylamino)- | [M + H]⁺ |
| | | propanoate | |

¹Compound 197: ¹H NMR (400 MHz, DMSO-d₆) δ ppm 1.90-2.09 (m, 2H), 2.73-3.68 (m, 16H), 3.73 (s, 4H), 3.79-4.14 (m, 3H), 7.29 (dd, J = 8.84, 2.27 Hz, 1H), 7.35-7.45 (m, 3H), 7.93 (t, J = 8.02 Hz, 1H), 8.29 (d, J = 8.46 Hz, 1H), 9.69 (d, J = 8.84 Hz, 1H).

### Example 1.122

### Preparation of (R)-2-(4-(4-Chloro-2-(4-(3,3,3-trifluoropropyl)piperazin-1-yl)phenylcarbamoyl)-3-fluorobenzylamino)-3-hydroxypropanoic acid (Compound 199)

Intermediate 2 (50 mg, 0.096 mmol), (R)-methyl 2-amino-3-hydroxypropanoate hydrochloride (14.88 mg, 0.096 mmol), and DIEA (16.70 μL, 0.096 mmol) were dissolved in DMF (0.5 mL). The reaction was heated to 80° C. and stirred at this temperature for 3 h. After this time, the reaction was substantially complete. The reaction was extracted (2 mL each of H₂O/NaHCO₃ and MTBE). The organic layers were combined, dried, and the solvent was removed. The resulting oil was re-dissolved in THF (0.5 mL). LiOH (6.87 mg, 0.287 mmol) and H₂O (0.2 mL) were added to the solution. The reaction was stirred overnight at room temperature The mixture was purified by preparative LC/MS (5-75% ACN/H₂O (0.1% TFA)) to give the TFA salt of the title compound (27.5 mg, 36.7%) as a white solid. LC/MS m/z=547.4 [M+H]⁺; ¹H NMR (400 MHz, DMSO-d₆) δ ppm 2.62-2.80 (m, 3H), 3.06 (m, 11H), 3.93 (s, 2H), 3.98 (s, 1H), 4.30 (s, 2H), 5.63 (bs, 1H), 7.28 (dd, T=8.78, 2.21 Hz, 1H), 7.36-7.41 (m, 1H), 7.51 (dd, T=8.02, 0.95 Hz, 1H), 7.58 (d, T=12.38 Hz, 1H), 7.98 (t, T=7.96 Hz, 1H), 8.28 (d, J=8.21 Hz, 1H), 9.75 (d, T=8.72 Hz, 1H).

### Example 1.123

### Preparation of (R)-4-(1-Amino-3-hydroxy-1-oxopropan-2-ylamino)methyl)-N-(4-chloro-2-(4-(3,3,3-trifluoropropyl)piperazin-1-yl)phenyl)-2-fluorobenzamide (Compound 195)

### Step A: Preparation of (R)-2-amino-3-hydroxypropanamide Hydrochloride

(R)-2-(tert-Butoxycarbonylamino)-3-hydroxypropanoic acid (200 mg, 0.975 mmol) and HATU (445 mg, 1.170 mmol) were dissolved in DMF (3 mL). Then, ammonia (21.09 μL, 0.975 mmol) (gas, from cylinder) was bubbled into the solution at room temperature (the solution quickly turned yellow, and after 30 seconds a yellow precipitate formed). The reaction was extracted (10 mL each of H₂O and MTBE). The organic layers were combined, dried, and concentrated to give the Boc protected intermediate, (R)-tert-butyl 1-amino-3-hydroxy-1-oxopropan-2-ylcarbamate, as a colorless oil. The intermediate was re-dissolved in CH₂Cl₂ (3 mL) and HCl (4 M in dioxane) (487 μL, 1.949 mmol) was added at room temperature. The reaction was stirred with heating (50° C.) for 15 min. A precipitate was formed. The solution was cooled to room temperature and the precipitate was filtered, washed with MTBE, and dried to give the HCl salt of the title compound (25 mg, 17.88%), as a white solid. LC/MS m/z=105.0 [M+H]⁺; ¹H NMR (400 MHz, DMSO-d₆) δ ppm 3.67-3.81 (m, 3H), 5.46 (bs, 1H), 7.52 (s, 1H), 7.84 (s, 1H), 8.09 (s, 3H).

### Step B: Preparation of (R)-4-(1-Amino-3-hydroxy-1-oxopropan-2-ylamino)methyl)-N-(4-chloro-2-(4-(3,3,3-trifluoropropyl)piperazin-1-yl)phenyl)-2-fluorobenzamide (Compound 195) as the Hydrochloride Salt

Intermediate 2 (15 mg, 0.029 mmol), (R)-2-amino-3-hydroxypropanamide hydrochloride (4.03 mg, 0.029 mmol), and DIEA (15.03 μL, 0.086 mmol) were added to a vial with DMF (0.2 mL). The reaction was stirred with heating to 80° C. for 3 h. The mixture was purified by preparative LC/MS (5-70% ACN/H₂O (0.1% TFA), 25 min). After lyophilization, the product was obtained as a TFA salt. It was converted to its corresponding HCl salt by dissolving the precipitate in ACN (0.5 mL) and H₂O (1 mL), and then adding 5 M HCl (5 eq). The solution was stirred for 1 h, frozen and lyophilized to give the HCl salt of the title compound (5.0 mg, 7.72 μmol, 26.9%), as an off white solid. LC/MS m/z=546.4 [M+H]⁺.

### Example 1.124

### Preparation of 4-(((2H-Tetrazol-5-yl)methylamino)methyl)-N-(4-chloro-2-(4-(3,3,3-trifluoropropyl)piperazin-1-yl)phenyl)-2,3-difluorobenzamide (Compound 200)

Intermediate 1 (10 mg, 0.018 mmol), (2H-tetrazol-5-yl)methanamine (1.833 mg, 0.018 mmol), and DIEA (3.23 μL, 0.018 mmol) were added to a vial with DMF (0.2 mL). The reaction was stirred with heating at 80° C. for 2 h. The mixture was purified by preparative LC/MS (5-70% ACN/H₂O, 25 min) to give the title compound (1.0 mg, 1.245 μmol, 6.73%), as a white solid. LC/MS m/z=559.0 [M+H]⁺.

### Example 1.125

### Preparation of N-(4-Chloro-2-(4-(3,3,3-trifluoropropyl)piperazin-1-yl)phenyl)-2,3-difluoro-4-(((3-oxo-2,3-dihydroisoxazol-5-yl)methylamino)methyl)benzamide (Compound 201)

From Intermediate 1 and 5-(aminomethyl)isoxazol-3(2H)-one, using a similar method to the one described in Example 1.124, the title compound was obtained. LC/MS m/z=574.4 [M+H]⁺; ¹H NMR (400 MHz, DMSO-d₆) δ ppm 2.79-2.96 (m, 3H), 3.01-3.85 (m, 10H), 4.31-4.39 (m, 4H), 6.24 (s, 1H), 7.28 (dd, J=8.84, 2.15 Hz, 1H), 7.36 (s, 1H), 7.53 (dd, J=7.96, 4.29 Hz, 1H), 7.71 (dd, J=7.83, 5.05 Hz, 1H), 8.13 (d, J=8.08 Hz, 1H), 9.80 (s, 1H), 11.55 (bs, 1H).

### Example 1.126

### Preparation of N-(4-Chloro-2-(4-(3,3,3-trifluoropropyl)piperazin-1-yl)phenyl)-4-(diethylamino)benzamide (Compound 34)

To a solution of 4-chloro-2-(4-(3,3,3-trifluoropropyl)piperazin-1-yl)aniline (40 mg, 0.13 mmol) and 4-(diethylamino)benzoic acid (30 mg, 0.16 mmol) in CH₃CN (5 mL) were added bis(2-oxo-3-oxazolidinyl)phosphinic chloride (50 mg, 0.20 mmol) and triethylamine (0.036 mL, 0.26 mmol). The reaction was stirred at 80° C. for 15 h. The mixture was concentrated. The residue was purified by HPLC to give the title compound. LC/MS m/z=483.2 [M+H]⁺.

### Example 1.127

### Preparation of 2-(4-(4-Chloro-2-(4-(3,3,3-trifluoropropyl)piperazin-1-yl)phenylcarbamoyl)-2,3-difluorobenzylamino)acetic acid (Compound 202)

Intermediate 1 (10 mg, 0.018 mmol), methyl 2-aminoacetate (1.648 mg, 0.018 mmol), and DIEA (3.23 μL, 0.018 mmol) were added to a vial with DMF (0.2 μL). The reaction was stirred with heating 80° C. for 2 h. LiOH (1.329 mg, 0.055 mmol) and H₂O (0.1 mL) were added to the reaction to cleave the intermediate ester. The reaction was stirred with slight heating 50° C. until the ester was cleaved. The mixture was purified by preparative LC/MS (5-70% ACN/H₂O, 25 min) to give the TFA salt of the title compound (3.7 mg, 4.80 μmol, 26.0%) as a white solid. LC/MS m/z=535.4 [M+H]⁺; ¹H NMR (400 MHz, DMSO-d₆) δ ppm 2.77-2.90 (m, 3H), 3.07-3.71 (m, 11H), 3.97 (s, 2H), 4.34 (s, 2H), 7.28 (dd, J=8.78, 2.21 Hz, 1H), 7.36 (s, 1H), 7.54 (d, J=7.96 Hz, 1H), 7.72 (d, J=7.45 Hz, 1H), 8.14 (d, J=8.34 Hz, 1H), 9.81 (s, 1H).

### Example 1.128

### Preparation of 4-(4-(4-Chloro-2-(4-(3,3,3-trifluoropropyl)piperazin-1-yl)phenylcarbamoyl)-2,3-difluorobenzylamino)butanoic acid (Compound 203)

From Intermediate 1 and methyl 4-aminobutanoate, using a similar method to the one described in Example 1.127, the title compound was obtained. LC/MS m/z=563.4 [M+H]⁺.

### Example 1.129

### Preparation of N-(4-Chloro-2-(4-(3,3,3-trifluoropropyl)piperazin-1-yl)phenyl)-4-(1H-pyrrol-1-yl)benzamide (Compound 46)

From 4-chloro-2-(4-(3,3,3-trifluoropropyl)piperazin-1-yl)aniline and 4-(1H-pyrrol-1-yl)benzoic acid, using a similar method to the one described in Example 1.126, the title compound was obtained. LC/MS m/z=477.1 [M+H]⁺.

### Example 1.130

### Preparation of (S)-2-(4-(4-Chloro-2-(4-(3,3,3-trifluoropropyl)piperazin-1-yl)phenylcarbamoyl)-2,3-difluorobenzylamino)-3-hydroxypropanoic acid (Compound 204)

From Intermediate 1 and (S)-methyl 2-amino-3-hydroxypropanoate hydrochloride, using a similar method to the one described in Example 1.127, the title compound was obtained. LC/MS m/z=565.2 [M+H]⁺; ¹H NMR (400 MHz, DMSO-d₆) δ ppm 2.46-2.58 (m, 2H), 2.73-2.85 (m, 3H), 2.94-3.78 (m, 10H), 3.93 (s, 2H), 4.04 (s, 1H), 4.33 (s, 2H), 7.25-7.30 (m, 1H), 7.36 (s, 1H), 7.55-7.60 (m, 1H), 7.69-7.74 (m, 1H), 8.15 (d, J=8.46 Hz, 1H), 9.79 (d, J=4.42 Hz, 1H).

### Example 1.131

### Preparation of 2-(1-(4-(4-Chloro-2-(4-(3,3,3-trifluoropropyl)piperazin-1-yl)phenylcarbamoyl)-2,3-difluorobenzyl)pyrrolidin-3-yl)acetic acid (Compound 206)

Intermediate 1 (10 mg, 0.018 mmol), tert-butyl 2-(pyrrolidin-3-yl)acetate (4.1 mg, 0.022 mmol), and DIEA (9.69 μL, 0.055 mmol) were added to a vial with DMF (0.2 mL). The reaction was heated to ˜80° C. for 1 h. After this time, the reaction was complete. The mixture was purified by preparative LC/MS (5-75% ACN/H₂O, 25 min). The resulting intermediate was dissolved in CH₂Cl₂ (0.5 mL) and TFA (0.5 mL). The mixture was stirred at room temperature for 2 h. The solvent was evaporated and the resulting product was dissolved in ACN (0.5 mL) and H₂O (1 mL). HCl (5M in H₂O) was added (6 eq). The reaction was frozen and lyophilized to yield the title compound (11.9 mg, 96%). LC/MS m/z=589.4 [M+H]⁺; ¹H NMR (400 MHz, DMSO-d₆) δ ppm 1.57-1.85 (m, J=48.88 Hz, 1H), 2.11-2.35 (m, 1H), 2.54-2.69 (m, 1H), 2.72-2.92 (m, 1H), 2.90-3.09 (m, 3H), 3.10-3.28 (m, 8H), 3.44-3.56 (m, 3H), 3.56-3.74 (m, 3H), 4.54 (s, 2H), 7.28 (dd, J=8.65, 2.21 Hz, 1H), 7.33 (s, 1H), 7.72 (s, 2H), 8.11 (d, J=8.97 Hz, 1H), 9.83 (s, 1H).

### Examples 1.132 to 1.143

The following compounds were prepared using the disclosed intermediates and a method similar to the one described in Example 1.131.

| | | | Product |
|---|---|---|---|
| Example | Cmpd | | (Mass Observed |
| No. | No. | Intermediates Used | LC/MS m/z) |
| 1.132 | 207¹ | Intermediate 1 and tert-butyl 3- | 549.4 [M + H]⁺ |
| | | aminopropanoate hydrochloride | |
| 1.133 | 208 | Intermediate 1 and (S)-tert-butyl 2,4-diamino- | 592.4 [M + H]⁺ |
| | | 4-oxobutanoate | |
| 1.134 | 209 | Intermediate 1 and (S)-tert-butyl 2-amino-4- | 591.4 [M + H]⁺ |
| | | methylpentanoate hydrochloride | |
| 1.135 | 210 | Intermediate 1 and tert-butyl 2- | 549.2 [M + H]⁺ |
| | | (methylamino)acetate dihydrochloride | |
| 1.136 | 211 | Intermediate 1 and (S)-di-tert-butyl 2- | 607.8 [M + H]⁺ |
| | | aminopentanedioate hydrochloride | |
| 1.137 | 212 | Intermediate 1 and tert-butyl 2-amino-2- | 563.4 [M + H]⁺ |
| | | methylpropanoate hydrochloride | |
| 1.138 | 213 | Intermediate 1 and (S)-5-tert-butyl 1-methyl 2- | 621.6 [M + H]⁺ |
| | | aminopentanedioate hydrochloride | |
| 1.139 | 214 | Intermediate 1 and (S)-4-tert-butyl 1-methyl 2- | 607.6 [M + H]⁺ |
| | | aminosuccinate hydrochloride | |
| 1.140 | 215 | Intermediate 1 and tert-butyl 4- | 560.4 [M + H]⁺ |
| | | aminopiperidine-1-carboxylate | |
| 1.141 | 216 | Intermediate 1 and tert-butyl 2,7- | 586.2 [M + H]⁺ |
| | | diazaspiro[3.5]nonane-7-carboxylate | |
| 1.142 | 217 | Intermediate 1 and (R)-tert-butyl 3- | 560.4 [M + H]⁺ |
| | | aminopiperidine-1-carboxylate | |
| 1.143 | 218 | Intermediate 1 and (R)-morpholin-2-ylmethanol | 577.4 [M + H]⁺ |
| | | hydrochloride | |

¹Compound 207: ¹H NMR (400 MHz, DMSO-d₆) δ ppm 1.19-1.37 (m, 5H), 2.76 (t, J = 7.33 Hz, 2H), 2.83-3.06 (m, 3H), 3.08-3.25 (m, 4H), 3.53-3.73 (m, 2H), 4.35 (s, 2H), 7.22-7.38 (m, 2H), 7.54-7.79 (m, 2H), 8.12 (d, J = 8.21 Hz, 1H), 9.28-9.62 (m, 2H), 9.82 (s, 1H), 11.35 (bs, 1H), 12.70 (bs, 1H).

### Example 1.144

### Preparation of 2-(1-(4-(4-Chloro-2-(4-(3,3,3-trifluoropropyl)piperazin-1-yl)phenylcarbamoyl)-2,3-difluorobenzyl)piperazin-2-yl) acetic Acid (Compound 226)

### Step A: Preparation of 2-(4-(tert-Butoxycarbonyl)-1-(4-(4-chloro-2-(4-(3,3,3-trifluoropropyl)piperazin-1-yl)phenylcarbamoyl)-2,3-difluorobenzyl)piperazin-2-yl)acetic Acid (Compound 219)

Intermediate 1 (10.0 mg, 0.018 mmol), tert-butyl 3-(2-methoxy-2-oxoethyl)piperazine-1-carboxylate (5.73 mg, 0.022 mmol), and DIEA (20.00 μL, 0.115 mmol) were dissolved in DMF (0.2 mL). The reaction was heated to ˜80° C. with stirring for 1 h to provide tert-butyl 4-(4-(4-chloro-2-(4-(3,3,3-trifluoropropyl)piperazin-1-yl)phenylcarbamoyl)-2,3-difluorobenzyl)-3-(2-methoxy-2-oxoethyl)piperazine-1-carboxylate. To the reaction mixture was added LiOH (2M in H₂O, 92 μL, 0.185 mmol) and H₂O (100 μL). Essentially no reaction occurred at room temperature. The reaction mixture was heat to ˜80-100° C. with stirring until complete. The reaction was made slightly acidic with HCl (5 M in H₂O). The mixture was purified by preparative HPLC (5-70% ACN/H₂O (0.1% TFA)) to give the TFA salt of the title compound (13.2 mg, 77%). LC/MS m/z=704.4 [M+H]⁺.

### Step B: Preparation of 2-(1-(4-(4-Chloro-2-(4-(3,3,3-trifluoropropyl)piperazin-1-yl)phenylcarbamoyl)-2,3-difluorobenzyl)piperazin-2-yl) acetic Acid (Compound 226)

The TFA salt of 2-(4-(tert-butoxycarbonyl)-1-(4-(4-chloro-2-(4-(3,3,3-trifluoropropyl)piperazin-1-yl)phenylcarbamoyl)-2,3-difluorobenzyl)piperazin-2-yl)acetic acid (13.2 mg, 0.014 mmol) was dissolved in CH₂Cl₂ (0.5 mL) and TFA (0.2 mL). The reaction was stirred at room temperature overnight. The next day, the solvent was removed and the residue was purified by preparative HPLC (5-70% ACN/H₂O) to give the title compound (11.7 mg, 63.5%). LC/MS m/z=604.6 [M+H]⁺.

### Examples 1.145 to 1.158

The following compounds were prepared using the disclosed intermediates and a method similar to the one described in Example 1.144, Step A or Example 1.144, Step B.

| | | | Product |
|---|---|---|---|
| Example | Cmpd | | (Mass Observed |
| No. | No. | Intermediates Used | LC/MS m/z) |
| 1.145 | 220 | Intermediate 1 and 1-tert-butyl 3-methyl | 690.6 [M + H]⁺ |
| | | piperazine-1,3-dicarboxylate | |
| 1.146 | 221 | Intermediate 1 and (S)-methyl 2-amino-4-(tert- | 678.4 [M + H]⁺ |
| | | butoxycarbonylamino)butanoate dihydrochloride | |
| 1.147 | 222 | Intermediate 1 and (R)-methyl 2-amino-3-(tert- | 664.6 [M + H]⁺ |
| | | butoxycarbonylamino)propanoate | |
| | | dihydrochloride | |
| 1.148 | 223 | Intermediate 1 and (S)-tert-butyl 3-(2-methoxy- | 703.6 [M + H]⁺ |
| | | 2-oxoethyl)piperazine-1-carboxylate | |
| 1.149 | 224 | Intermediate 1 and ethyl piperidine-4- | 589.6 [M + H]⁺ |
| | | carboxylate | |
| 1.150 | 225 | Intermediate 1 and methyl 2-(morpholin-2- | 605.6 [M + H]⁺ |
| | | yl)acetate | |
| 1.151 | 227 | 4-(tert-butoxycarbonyl)-1-(4-(4-chloro-2-(4- | 590.4 [M + H]⁺ |
| | | (3,3,3-trifluoropropyl)piperazin-1- | |
| | | yl)phenylcarbamoyl)-2,3- | |
| | | difluorobenzyl)piperazine-2-carboxylic acid | |
| 1.152 | 228 | Intermediate 1 and (R)-methyl 2-amino-3- | 565.4 [M + H]⁺ |
| | | hydroxypropanoate hydrochloride | |
| 1.153 | 229 | (S)-4-(tert-butoxycarbonylamino)-2-(4-(4- | 578.4 [M + H]⁺ |
| | | chloro-2-(4-(3,3,3-trifluoropropyl)piperazin-1- | |
| | | yl)phenylcarbamoyl)-2,3- | |
| | | difluorobenzylamino)butanoic acid (TFA salt) | |
| 1.154 | 230 | (R)-3-(tert-butoxycarbonylamino)-2-(4-(4- | 564.4 [M + H]⁺ |
| | | chloro-2-(4-(3,3,3-trifluoropropyl)piperazin-1- | |
| | | yl)phenylcarbamoyl)-2,3- | |
| | | difluorobenzylamino)propanoic acid (TFA salt) | |
| 1.155 | 231 | (S)-2-(4-(tert-butoxycarbonyl)-1-(4-(4-chloro-2- | 603.4 [M + H]⁺ |
| | | (4-(3,3,3-trifluoropropyl)piperazin-1- | |
| | | yl)phenylcarbamoyl)-2,3- | |
| | | difluorobenzyl)piperazin-2-yl)acetic acid | |
| 1.156 | 232 | Intermediate 1 and ethyl 2-(piperazin-1- | 604.6 [M + H]⁺ |
| | | yl)acetate | |
| 1.157 | 233 | Intermediate 1 and methyl 6-aminohexanoate | 591.2 [M + H]⁺ |
| | | hydrochloride | |
| 1.158 | 234 | Intermediate 1 and ethyl 5,6,7,8- | 627.6 [M + H]⁺ |
| | | tetrahydroimidazo[1,2-a]pyrazine-2-carboxylate | |
| | | dihydrochloride hydrate | |

### Example 1.159

### Preparation of N-(4-Chloro-2-(4-(3,3,3-trifluoropropyl)piperazin-1-yl)phenyl)-4-(2-cyanoethylamino)methyl)-2,3-difluorobenzamide (Compound 235)

Intermediate 1 (61.7 mg, 0.114 mmol), 3-aminopropanenitrile (9.60 mg, 0.137 mmol), and DIEA (77 μL, 0.442 mmol) were dissolved/suspended in DMF (0.4 mL). The reaction was stirred with heating to ˜100° C. for 1 h. After this time, the reaction was complete. The reaction mixture was purified by preparative LC/MS (5-70% ACN/H₂O, 25 min) to yield the title compound (26 mg, 42.6%). LC/MS m/z=530.2 [M+H]⁺.

### Example 1.160

### Preparation of N-(4-Chloro-2-(4-(3,3,3-trifluoropropyl)piperazin-1-yl)phenyl)-4-(pyrrolidin-1-ylmethyl)benzamide (Compound 44)

From 4-chloro-2-(4-(3,3,3-trifluoropropyl)piperazin-1-yl)aniline and 4-(pyrrolidin-1-ylmethyl)benzoic acid, using a similar method to the one described in Example 1.126, the title compound was obtained. LC/MS m/z=495.1 [M+H]⁺.

### Example 1.161

### Preparation of N-(4-Chloro-2-(4-(3,3,3-trifluoropropyl)piperazin-1-yl)phenyl)-4-(1H-pyrazol-1-yl)benzamide (Compound 45)

From 4-chloro-2-(4-(3,3,3-trifluoropropyl)piperazin-1-yl)aniline and 4-(1H-pyrazol-1-yl)benzoic acid, using a similar method to the one described in Example 1.126, the title compound was obtained. LC/MS m/z=478.1 [M+H]⁺.

### Example 1.162

### Preparation of N-(4-Chloro-2-(4-(3,3,3-trifluoropropyl)piperazin-1-yl)phenyl)-2-(2-(methylsulfonyl)ethyl)-1,2,3,4-tetrahydroisoquinoline-7-carboxamide (Compound 36)

### Step A. Preparation of N-(4-chloro-2-(4-(3,3,3-trifluoropropyl)piperazin-1-yl)phenyl)-1,2,3,4-tetrahydroisoquinoline-7-carboxamide

To a solution of 4-chloro-2-(4-(3,3,3-trifluoropropyl)piperazin-1-yl)aniline (0.25 g, 0.812 mmol) in DMF (2 mL), were added 2-(tert-butoxycarbonyl)-1,2,3,4-tetrahydroisoquinoline-7-carboxylic acid (0.225 g, 0.812 mmol), HATU (0.309 g, 0.812 mmol), and DIEA (0.105 g, 0.812 mmol) at ambient temperature. After stirring for 12 h, the reaction was poured into water and extracted with ethyl acetate. The ethyl acetate layer was dried over MgSO₄ and concentrated under reduced pressure to furnish the crude tert-butyl 7-(4-chloro-2-(4-(3,3,3-trifluoropropyl)piperazin-1-yl)phenylcarbamoyl)-3,4-dihydroisoquinoline-2(1H)-carboxylate. LC/MS m/z=566.6 [M+H]⁺. The crude compound was treated with 4.0 M HCl in dioxane (1 mL) for 5 h and concentrated under reduced pressure to give the HCl salt of N-(4-chloro-2-(4-(3,3,3-trifluoropropyl)piperazin-1-yl)phenyl)-1,2,3,4-tetrahydroisoquinoline-7-carboxamide (0.30 g, 81.6%). LC/MS m/z=466.6 [M+H]⁺.

### Step B. Preparation of N-(4-Chloro-2-(4-(3,3,3-trifluoropropyl)piperazin-1-yl)phenyl)-2-(2-(methylsulfonyl)ethyl)-1,2,3,4-tetrahydroisoquinoline-7-carboxamide (Compound 36)

To a solution of N-(4-chloro-2-(4-(3,3,3-trifluoropropyl)piperazin-1-yl)phenyl)-1,2,3,4-tetrahydroisoquinoline-7-carboxamide dihydrochloride (15 mg, 0.028 mmol) in DMF (2 mL), were added methylsulfonylethene (2.95 mg, 0.028 mmol) and DIEA (3.59 mg, 0.028 mmol) at room temperature. After stirring for 30 min, the reaction was heated to 100° C. for 30 min. The reaction was purified by HPLC to give the title compound, N-(4-chloro-2-(4-(3,3,3-trifluoropropyl)piperazin-1-yl)phenyl)-2-(2-(methylsulfonyl)ethyl)-1,2,3,4-tetrahydroisoquinoline-7-carboxamide (11.2 mg, 70.3%). LC/MS m/z=573.5 [M+H]⁺.

### Example 1.163

### Preparation of 4-(Aminomethyl)-N-(4-bromo-2-(4-(3,3,3-trifluoropropyl)piperazin-1-yl)phenyl)-2,3-difluorobenzamide (Compound 238)

### Step A: Preparation of 1-(5-bromo-2-nitrophenyl)piperazine

Piperazine (5.87 g, 68.2 mmol) was dissolved in IPA (50 mL) and cooled on an ice bath. 4-bromo-2-fluoro-1-nitrobenzene (5.0 g, 22.73 mmol), pre-dissolved in IPA (30 mL; heating briefly with a heat gun to fully dissolve) was added to the solution via an addition funnel. Upon complete addition, the reaction was warmed to room temperature and stirred over night. The next day, the solvent was removed and the reaction partitioned (3×200 mL each of H₂O and EtOAc). The organic layers were combined and back extracted with H₂O (500 mL). The organic layer was dried and concentrated to yield the title compound (6.5 g, 99%). LC/MS m/z=286.2 [M+H]⁺; ¹H NMR (400 MHz, DMSO-d₆) δ ppm 2.74-2.80 (m, 4H), 2.90-2.97 (m, 4H), 7.22 (dd, J=8.59, 2.02 Hz, 1H), 7.40 (d, J=1.89 Hz, 1H), 7.74 (d, J=8.59 Hz, 1H).

### Step B: Preparation of 4-Bromo-2-(4-(3,3,3-trifluoropropyl)piperazin-1-yl)aniline

1-(5-Bromo-2-nitrophenyl)piperazine (6.5 g, 22.72 mmol) was dissolved in THF (50 mL) and MeOH (10 mL). 3-Bromo-1,1,1-trifluoropropane (4.86 mL, 45.4 mmol) and DIEA (3.97 mL, 22.72 mmol) were added and the reaction was heated to reflux over the weekend. Then the solvent was evaporated and the resulting oil was re-dissolved in EtOH (40 mL). Tin(II) chloride (12.92 g, 68.2 mmol) was added in two portions. Then the reaction was heated to 80° C. in an oil bath and stirred at this temperature for 4 h. After this time, the reaction was complete. The reaction was cooled and quenched with the addition of H₂O (20 mL) and conc. NaOH (50 wt %; 20 mL). The reaction was extracted (3×400 mL DCM and H₂O/NaOH). The organic layers were combined, dried, concentrated, and the residue was purified by column chromatography (0-50% EtOAc/hexanes) to yield the title compound (7.2 g, 87%). LC/MS m/z=352.2 [M+H]⁺; ¹H NMR (400 MHz, DMSO-d₆) δ ppm 2.47-2.55 (m, 2H), 2.59-2.70 (m, 6H), 2.78-2.90 (m, 4H), 4.91 (s, 2H), 6.69 (d, J=8.90 Hz, 1H), 6.98-7.03 (m, 2H).

### Step C: Preparation of 4-(Aminomethyl)-N-(4-bromo-2-(4-(3,3,3-trifluoropropyl)piperazin-1-yl)phenyl)-2,3-difluorobenzamide (Compound 238)

4-Bromo-2-(4-(3,3,3-trifluoropropyl)piperazin-1-yl)aniline (300 mg, 0.852 mmol), 4-((tert-butoxycarbonylamino)methyl)-2,3-difluorobenzoic acid (245 mg, 0.852 mmol), and DIEA (298 μL, 1.704 mmol) were dissolved in DMF (2 mL). HATU (389 mg, 1.022 mmol) was added and the reaction was stirred with heating to −80° C. for 1 h. After this time, the starting material was consumed. The reaction was extracted (5 mL each of H₂O and MTBE/EtOAc). The organic layers were combined, dried, concentrated, and the intermediate (i.e., tert-butyl 4-(4-bromo-2-(4-(3,3,3-trifluoropropyl)piperazin-1-yl)phenylcarbamoyl)-2,3-difluorobenzylcarbamate) was purified by column chromatography (0-20% EtOAc/hexanes). The fractions containing the intermediate were reduced to yield a yellow oil and dissolved in ACN (2 mL). HCl (4M in dioxane, 518 μL, 17.04 mmol) was added and the reaction was stirred at room temperature over night (to remove the Boc-protecting group). The next day, the resulting precipitate was filtered, washed with ACN, and dried under reduced pressure in an oven (60° C.) to yield the title compound (406 mg, 79%). LC/MS m/z=521.4 [M+H]⁺; ¹H NMR (400 MHz, DMSO-d₆) δ ppm 2.90-3.10 (m, 2H), 3.13-3.30 (m, 6H), 3.39-3.53 (m, 2H), 3.53-3.72 (m, 2H), 4.19 (s, 2H), 7.37-7.49 (m, 2H), 7.56 (t, J=7.20 Hz, 1H), 7.70 (t, J=7.07 Hz, 1H), 8.08 (d, J=8.21 Hz, 1H), 8.65 (bs, 3H), 9.81 (d, J=3.92 Hz, 1H), 11.73 (bs, 1H).

### Example 1.164

### Preparation of 4-(4-(4-Chloro-2-(4-(3,3,3-trifluoropropyl)piperazin-1-yl)phenylcarbamoyl)-2,3-difluorobenzyl)piperazine-1-carboxamide (Compound 243)

Intermediate 1 (10 mg, 0.020 mmol), piperazine-1-carboxamide (3.1 mg, 0.024 mmol), and DIEA (3.52 μL, 0.020 mmol) were dissolved in a vial with DMF (0.2 mL). The reaction was stirred with heating to ˜80° C. for 1 h. After this time, the reaction was complete. The reaction mixture was purified by preparative HPLC (5-50% ACN/H₂O, 25 min) to yield the title compound (14.1 mg, 85%). LC/MS m/z=589.4 [M+H]⁺; ¹H NMR (400 MHz, DMSO-d₆) δ ppm 2.75-2.94 (m, 4H), 2.95-3.07 (m, 4H), 3.09-3.28 (m, 13H), 4.28 (s, 2H), 6.16 (bs, 1H), 7.28 (dd, J=8.72, 2.27 Hz, 1H), 7.37 (d, J=1.89 Hz, 1H), 7.49-7.56 (m, 1H), 7.67-7.76 (m, 1H), 8.15 (d, J=8.46 Hz, 1H), 9.79 (d, J=3.41 Hz, 1H).

### Examples 1.165 to 1.167

The following compounds were prepared using the disclosed intermediates and a method similar to the one described in Example 1.164.

| | | | Product |
|---|---|---|---|
| | | | (Mass |
| Example | Cmpd | | Observed |
| No. | No. | Intermediates Used | LC/MS m/z) |
| 165 | 244 | Intermediate 1 and piperidine- | 588.4 [M + H]⁺ |
| | | 3-carboxamide | |
| 166 | 245 | Intermediate 1 and piperidine- | 588.6 [M + H]⁺ |
| | | 4-carboxamide | |
| 167 | 246 | Intermediate 1 and (R)-pyrrolidine- | 574.4 [M + H]⁺ |
| | | 2-carboxamide | |

### Example 1.168

### Preparation of (R)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 247)

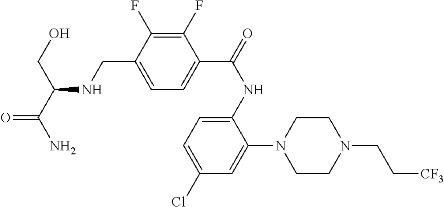 [SMILES: NC(=O)[C@@H](CO)NCc1ccc(C(=O)Nc2ccc(Cl)cc2N2CCN(CCC(F)(F)F)CC2)c(F)c1F]

From Intermediate 1 and (R)-2-amino-3-hydroxypropanamide hydrochloride, using a similar method to the one described in Example 1.164, the title compound was obtained. LC/MS m/z=564.4 [M+H]⁺; ¹H NMR (400 MHz, DMSO-d₆) δ ppm 2.87-3.08 (m, 3H), 3.11-3.33 (m, 10H), 3.78-3.96 (m, 3H), 4.31 (d, J=5.31 Hz, 2H), 7.24-7.38 (m, 2H), 7.60-7.73 (m, 3H), 7.99 (s, 1H), 8.12 (d, T=8.21 Hz, 1H), 9.43 (bs, 1H), 9.58 (bs, 1H), 9.80 (d, T=3.79 Hz, 1H), 11.62 (bs, 1H).

### Example 1.169

### Preparation of (S)-4-(((1-Amino-3-hydroxy-1-oxopropan-2-yl)(ethyl)amino)methyl)-N-(4-chloro-2-(4-(3,3,3-trifluoropropyl)piperazin-1-yl)phenyl)-2,3-difluorobenzamide (Compound 248)

(S)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride (10 mg, 0.016 mmol), ethyl iodide (1.269 μL, 0.016 mmol), and DIEA (10.97 μL, 0.063 mmol) were dissolved in DMF (0.2 mL). The reaction was stirred with heating to ˜70° C. overnight. The next day, the reaction was around 40% complete. Thus, more ethyl iodide (10 eq.) was added. After the reaction was substantially complete, the mixture was purified by preparative HPLC (5-40% ACN/H₂O, 65 min). After lyophilization the product was converted to the corresponding HCl salt to yield the title compound (2.0 mg, 2.98 μmol, 18.96%). LC/MS m/z=592.6 [M+H]⁺.

### Example 1.170

### Preparation of (R)-3-Amino-2-(4-(4-chloro-2-(4-(3,3,3-trifluoropropyl)piperazin-1-yl)phenylcarbamoyl)-2,3-difluorobenzylamino)-3-oxopropyl Dihydrogenphosphate (Compound 250)

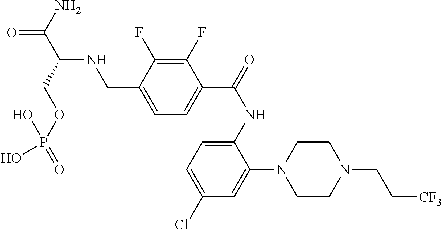 [SMILES: NC(=O)[C@@H](COP(=O)(O)O)NCc1ccc(C(=O)Nc2ccc(Cl)cc2N2CCN(CCC(F)(F)F)CC2)c(F)c1F]

### Step A: Preparation of (R)-3-amino-2-(4-(4-chloro-2-(4-(3,3,3-trifluoropropyl)piperazin-1-yl)phenylcarbamoyl)-2,3-difluorobenzylamino)-3-oxopropyl dihydrogen phosphate

(R)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (35 mg, 0.062 mmol) was dissolved in 1H-tetrazole (0.5 M in ACN) (248 μL, 0.124 mmol) and DCM (1 mL). Diallyl diisopropylphosphoramidite (45.7 mg, 0.186 mmol) was added. The reaction was stirred overnight at room temperature. The solvent was evaporated and the residue was purified by column chromatography (0-50% EtOAc/hexanes). The purified intermediate was dissolved in THF (2 mL), and tert-butyl hydroperoxide (7.22 μL, 0.074 mmol) was added. The reaction was stirred at room temperature for 4 h. The mixture was concentrated and the residue was re-dissolved in DCM and triphenylphosphine (1.628 mg, 6.21 μmol), tetrakis(triphenylphosphine)palladium (0) (3.59 mg, 3.10 μmol), and pyrrolidine (103 μL, 1.241 mmol) were added. The reaction was stirred at room temperature overnight. The mixture was concentrated and the residue was purified by HPLC (5-70% ACN/H₂O, 40 min) to give the TFA salt of the title compound (14.1 mg, 26%). LC/MS m/z=644.0 [M+H]⁺; ¹H NMR (400 MHz, DMSO-d₆) δ ppm 2.73-2.87 (m, 2H), 2.96-3.18 (m, 4H), 3.18-3.37 (m, 5H), 3.87-3.94 (m, 3H), 4.15-4.26 (m, 5H), 7.28 (dd, J=8.77, 2.16 Hz, 1H), 7.36 (d, J=1.78 Hz, 1H), 7.51-7.59 (m, 1H), 7.63-7.74 (m, 2H), 7.91 (s, 1H), 8.15 (d, J=7.88 Hz, 1H), 9.78 (d, J=4.07 Hz, 1H).

### Step B: Preparation of (R)-3-Amino-2-(4-(4-chloro-2-(4-(3,3,3-trifluoropropyl)piperazin-1-yl)phenylcarbamoyl)-2,3-difluorobenzylamino)-3-oxopropyl Dihydrogenphosphate (Compound 250)

The TFA salt of (R)-3-amino-2-(4-(4-chloro-2-(4-(3,3,3-trifluoropropyl)piperazin-1-yl)phenylcarbamoyl)-2,3-difluorobenzylamino)-3-oxopropyl dihydrogen phosphate (14.1 mg, 0.016 mol), was dissolved in NaOH (5 eq.) and H₂O (1 mL). The product was purified on a C18 reverse phase column (5-10% MeOH/H₂O) to give the sodium salt of the title compound (1.6 mg, 2.303 μmmol, 3.71%). LC/MS m/z=643.8 [M+H]⁺; ¹H NMR (400 MHz, D₂O) δ ppm 2.25-2.43 (m, 2H), 2.52-2.66 (m, 6H), 2.82 (s, 4H), 3.39 (t, J=5.72 Hz, 1H), 3.68-3.85 (m, 4H), 7.07 (dd, J=8.65, 2.03 Hz, 1H), 7.19-7.28 (m, 2H), 7.48 (t, J=7.25 Hz, 1H), 7.73 (d, J=8.65 Hz, 1H).

### Example 1.171

### Preparation of N-(4-Chloro-2-(4-(3,3,3-trifluoropropyl)piperazin-1-yl)phenyl)-4-(1H-1,2,4-triazol-1-yl)benzamide (Compound 43)

From 4-chloro-2-(4-(3,3,3-trifluoropropyl)piperazin-1-yl)aniline and 4-(1H-1,2,4-triazol-1-yl)benzoic acid, using a similar method to the one described in Example 1.126, the title compound was obtained. LC/MS m/z=479.1 [M+H]⁺.

### Example 1.172

### Preparation of 7-(4-Chloro-2-(4-(3,3,3-trifluoropropyl)piperazin-1-yl)phenylcarbamoyl)-2,2-dimethyl-1,2,3,4-tetrahydroisoquinolinium-2,2,2-trifluoroacetate

### Step A: Preparation of 2-tert-Butyl 7-methyl 3,4-dihydroisoquinoline-2,7(1H)-dicarboxylate

Methyl 1,2,3,4-tetrahydroisoquinoline-7-carboxylate hydrochloride (395 mg, 1.735 mmol) was suspended in DCM (3 mL). DIEA (909 μL, 5.20 mmol) was added. Boc anhydride (443 μL, 1.908 mmol) was added (vigorous bubbling was observed). The reaction was stirred at room temperature for 1 h. After this time, the solvent was evaporated and the residue was purified by column chromatography (0-5% EtOAc/hexanes) to give the title compound (450 mg, 85%). LC/MS m/z=292.2 [M+H]⁺; ¹H NMR (400 MHz, DMSO-d₆) δ ppm 1.43 (s, 9H), 2.84 (t, J=5.87 Hz, 2H), 3.56 (t, J=5.87 Hz, 2H), 3.84 (s, 3H), 4.56 (s, 2H), 7.31 (d, J=7.83 Hz, 1H), 7.75 (d, J=7.96 Hz, 1H), 7.77 (s, 1H).

### Step B: Preparation of 2-(tert-Butoxycarbonyl)-1,2,3,4-tetrahydroisoquinoline-7-carboxylic acid

2-tert-Butyl 7-methyl 3,4-dihydroisoquinoline-2,7(1H)-dicarboxylate (450 mg, 1.545 mmol) was dissolved in THF (2 mL) and MeOH (1 mL). LiOH (111 mg, 4.63 mmol) and H₂O (1 mL) were added. The reaction was heated to 60° C. and stirred at this temperature for 3 h. After this time the reaction was complete. The solvent was evaporated, until only largely H₂O remained. This aqueous layer was cooled on an ice bath and then made slightly acidic by the addition of 5 M HCl slowly dropwise. The reaction was extracted (10 mL each of H₂O and EtOAc). The aqueous layer was extracted again with EtOAc (10 mL). The organic layers were combined, dried, and concentrated to give a colorless oil that solidified under reduced pressure overnight. This solid precipitate was triturated in MTBE, filtered, and dried to give the title compound (260 mg, 58.3%). LC/MS m/z=278.2 [M+H]⁺; ¹H NMR (400 MHz, DMSO-d₆) δ ppm 1.43 (s, 9H), 2.83 (t, J=5.87 Hz, 2H), 3.56 (t, J=5.94 Hz, 2H), 4.55 (s, 2H), 7.28 (d, J=7.96 Hz, 1H), 7.70-7.75 (m, 2H), 12.84 (brs, 1H).

### Step C: Preparation of tert-butyl 7-(4-chloro-2-(4-(3,3,3-trifluoropropyl)piperazin-1-yl)phenylcarbamoyl)-3,4-dihydroisoquinoline-2(1H)-carboxylate

2-(tert-Butoxycarbonyl)-1,2,3,4-tetrahydroisoquinoline-7-carboxylic acid (260 mg, 0.938 mmol), 4-chloro-2-(4-(3,3,3-trifluoropropyl)piperazin-1-yl)aniline (289 mg, 0.938 mmol), DIEA (246 μL, 1.406 mmol), and HATU (428 mg, 1.125 mmol) were added to DCM (6 mL) and DMF (1 mL). The reaction was stirred with heating to reflux (˜50° C.) and stirred at this temperature for 4 h. After this time, an extraction was performed (10 mL each of H₂O and DCM). The aqueous layer was extracted again with DCM (10 mL). The organic layers were combined, dried, and concentrated. The residue was purified by chromatography (0, 10, 15, 30, 40% EtOAc/hexanes) to give the title compound (270 mg, 49.8%). LC/MS m/z=567.6 [M+H]⁺; ¹H NMR (400 MHz, DMSO-d₆) δ ppm 1.43 (s, 9H), 2.54-2.64 (m, 7H), 2.71-2.95 (m, 7H), 3.59 (t, J=5.87 Hz, 2H), 4.60 (s, 2H), 7.20 (dd, J=8.65, 2.34 Hz, 1H), 7.29 (d, J=2.40 Hz, 1H), 7.37 (d, J=8.34 Hz, 1H), 7.69-7.80 (m, 2H), 8.07 (d, J=8.59 Hz, 1H), 9.46 (s, 1H).

### Step D: Preparation of N-(4-Chloro-2-(4-(3,3,3-trifluoropropyl)piperazin-1-yl)phenyl)-1,2,3,4-tetrahydroisoquinoline-7-carboxamide (Compound 13)

tert-Butyl 7-(4-chloro-2-(4-(3,3,3-trifluoropropyl)piperazin-1-yl)phenylcarbamoyl)-3,4-dihydroisoquinoline-2(1H)-carboxylate (270 mg, 0.476 mmol) was dissolved in DCM (3 mL). HCl (4M in dioxane) (2381 μL, 9.52 mmol) was added to the solution and stirred at room temperature overnight. The next day, the reaction mixture was extracted under basic conditions (50 mL each of 3 M NaOH and DCM). The aqueous layer was extracted again with DCM (50 mL). The organic layers were combined, dried, and concentrated to give the title compound (190.2 mg, 82%). LC/MS m/z=467.2 [M+H]⁺; 1H NMR (400 MHz, DMSO-d₆) δ ppm 2.53-2.64 (m, 7H), 2.82 (t, J=5.75 Hz, 2H), 2.85-2.94 (m, 5H), 3.06 (t, J=5.94 Hz, 2H), 4.01 (s, 2H), 7.16-7.23 (m, 1H), 7.26-7.32 (m, 2H), 7.64 (s, 1H), 7.70 (dd, T=7.89, 1.58 Hz, 1H), 8.08 (d, T=8.72 Hz, 1H), 9.42 (s, 1H).

### Step E: Preparation of 7-(4-Chloro-2-(4-(3,3,3-trifluoropropyl)piperazin-1-yl)phenylcarbamoyl)-2,2-dimethyl-1,2,3,4-tetrahydroisoquinolinium2,2,2-trifluoroacetate

N-(4-Chloro-2-(4-(3,3,3-trifluoropropyl)piperazin-1-yl)phenyl)-1,2,3,4-tetrahydroisoquinoline-7-carboxamide (11 mg, 0.024 mmol), DIEA (12.34 μL, 0.071 mmol), and methyl iodide (2.2 μL, 0.035 mmol) were dissolved in DMF (00.1 mL). The reaction was stirred at room temperature overnight. The reaction was heated to 80° C. and stirred at this temperature for 6 h. The mixture was purified by preparative LC/MS (10-95% ACN/H₂O, 30 min) to give the title compound (3.4 mg, 4.43 μmol, 18.8%). LC/MS m/z=495.2 [M]⁺.

### Examples 1.173 to 1.182

The following compounds were prepared using the disclosed intermediates and a method similar to the one described in Example 1.172, Step E.

| | | | Product |
|---|---|---|---|
| Example | Cmpd | | (Mass Observed |
| No. | No. | Intermediates Used | LC/MS m/z) |
| 1.173 | 15¹ | N-(4-chloro-2-(4-(3,3,3- | 523.6 [M + H]⁺ |
| | | trifluoropropyl)piperazin-1-yl)phenyl)-1,2,3,4- | |
| | | tetrahydroisoquinoline-7-carboxamide and 1- | |
| | | bromobutane | |
| 1.174 | 16 | N-(4-chloro-2-(4-(3,3,3- | 525.6 [M + H]⁺ |
| | | trifluoropropyl)piperazin-1-yl)phenyl)-1,2,3,4- | |
| | | tetrahydroisoquinoline-7-carboxamide and 3- | |
| | | bromopropan-1-ol | |
| 1.175 | 17² | N-(4-chloro-2-(4-(3,3,3- | 551.2 [M + H]⁺ |
| | | trifluoropropyl)piperazin-1-yl)phenyl)-1,2,3,4- | |
| | | tetrahydroisoquinoline-7-carboxamide and 1- | |
| | | bromo-3,3-dimethylbutane | |
| 1.176 | 18 | N-(4-chloro-2-(4-(3,3,3- | 565.2 [M + H]⁺ |
| | | trifluoropropyl)piperazin-1-yl)phenyl)-1,2,3,4- | |
| | | tetrahydroisoquinoline-7-carboxamide and 4- | |
| | | (bromomethyl)tetrahydro-2H-pyran | |
| 1.177 | 19 | N-(4-chloro-2-(4-(3,3,3- | 525.6 [M + H]⁺ |
| | | trifluoropropyl)piperazin-1-yl)phenyl)-1,2,3,4- | |
| | | tetrahydroisoquinoline-7-carboxamide and 1- | |
| | | bromo-2-methoxyethane | |
| 1.178 | 20 | N-(4-chloro-2-(4-(3,3,3- | 538.4 [M + H]⁺ |
| | | trifluoropropyl)piperazin-1-yl)phenyl)-1,2,3,4- | |
| | | tetrahydroisoquinoline-7-carboxamide and 3- | |
| | | bromopropanamide | |
| 1.179 | 21 | N-(4-chloro-2-(4-(3,3,3- | 511.2 [M + H]⁺ |
| | | trifluoropropyl)piperazin-1-yl)phenyl)-1,2,3,4- | |
| | | tetrahydroisoquinoline-7-carboxamide and 2- | |
| | | bromoethanol | |
| 1.180 | 22 | N-(4-chloro-2-(4-(3,3,3- | 553.6 [M + H]⁺ |
| | | trifluoropropyl)piperazin-1-yl)phenyl)-1,2,3,4- | |
| | | tetrahydroisoquinoline-7-carboxamide and ethyl | |
| | | 2-bromoacetate | |
| 1.181 | 23 | N-(4-chloro-2-(4-(3,3,3- | 524.2 [M + H]⁺ |
| | | trifluoropropyl)piperazin-1-yl)phenyl)-1,2,3,4- | |
| | | tetrahydroisoquinoline-7-carboxamide and 2- | |
| | | bromoacetamide | |
| 1.182 | 24 | N-(4-chloro-2-(4-(3,3,3- | 506.4 [M + H]⁺ |
| | | trifluoropropyl)piperazin-1-yl)phenyl)-1,2,3,4- | |
| | | tetrahydroisoquinoline-7-carboxamide and 2- | |
| | | bromoacetonitrile | |

¹Compound 15: ¹H NMR (400 MHz, DMSO-d₆) δ ppm 1.34-1.47 (m, 2H), 1.71-1.84 (m, 1H), 2.78-2.96 (m, J = 23.12 Hz, 2H), 3.09-3.34 (m, 10H), 3.44-3.67 (m, 6H), 3.75-3.84 (m, J = 10.86 Hz, 1H), 4.34-4.47 (m, J = 6.32 Hz, 1H), 4.71 (d, J = 15.16 Hz, 1H), 7.28 (dd, J = 8.72, 2.27 Hz, 1H), 7.35 (d, J = 2.27 Hz, 1H), 7.47 (d, J = 8.08 Hz, 1H), 7.86 (s, 1H), 7.88-7.94 (m, 1H), 7.99 (d, J = 8.59 Hz, 1H), 9.55 (s, 1H), 10.15 (bs, 1H).
²Compound 17: ¹H NMR (400 MHz, DMSO-d₆) δ ppm 0.92-0.98 (m, 9H), 1.56-1.80 (m, 2H), 2.75-2.90 (m, 2H), 3.04-3.31 (m, 9H), 3.31-3.40 (m, 4H), 3.58-3.73 (m, 2H), 3.81 (dd, J = 8.34, 3.54 Hz, 1H), 4.37 (dd, J = 15.03, 6.95 Hz, 1H), 4.64-4.75 (m, J = 17.43 Hz, 1H), 7.26 (dd, J = 8.59, 2.27 Hz, 1H), 7.33 (d, J = 2.40 Hz, 1H), 7.45 (d, J = 8.08 Hz, 1H), 7.82 (s, 1H), 7.89 (dd, J = 8.02, 1.33 Hz, 1H), 7.95-7.98 (m, 1H), 9.51 (s, 1H), 10.07 (bs, 1H).

### Example 1.184

### Preparation of N-(4-Chloro-2-(4-(3,3,3-trifluoropropyl)piperazin-1-yl)phenyl)-2-(2-(methylsulfonyl)ethyl)isoindoline-5-carboxamide (Compound 37)

To a solution of N-(4-chloro-2-(4-(3,3,3-trifluoropropyl)piperazin-1-yl)phenyl)isoindoline-5-carboxamide dihydrochloride (15 mg, 0.029 mmol) in DMF (2 mL), was added methylsulfonylethene (3.03 mg, 0.029 mmol) at ambient temperature. After stirring at 70° C. for 2 h, the reaction was extracted with ethyl acetate. The extract was dried over MgSO₄ and concentrated under reduced pressure to give the title compound (10.2 mg, 64.0%). LC/MS m/z=559.3 [M+H]⁺.

### Example 1.185

### Preparation of 4-(Aminomethyl)-N-(4-chloro-2-(4-(3,3,3-trifluoropropyl)piperazin-1-yl)phenyl)-2-fluorobenzamide (Compound 26)

### Step A: Preparation of N-(4-chloro-2-(4-(3,3,3-trifluoropropyl)piperazin-1-yl)phenyl)-4-cyano-2-fluorobenzamide

To a suspension of 4-cyano-2-fluorobenzoic acid (0.537 g, 3.25 mmol) in CH₂Cl₂ (10 mL), was added oxalyl chloride (1.422 mL, 16.25 mmol) followed by few drops of DMF. After stirring for 2 h, the reaction was concentrated under reduced pressure. The resulting residue was dissolved in fresh CH₂Cl₂ (10 mL) and treated with 4-chloro-2-(4-(3,3,3-trifluoropropyl)piperazin-1-yl)aniline (1.0 g, 3.25 mmol) followed by triethylamine (0.329 g, 3.25 mmol) at ambient temperature. After stirring for 1 h, the reaction was washed with water, dried over MgSO₄, and then concentrated under reduced pressure. The residue was triturated with methanol and filtered to give the title compound (1.25 g, 85%). LC/MS m/z=455.3 [M+H]⁺.

### Step B: Preparation of 4-(Aminomethyl)-N-(4-chloro-2-(4-(3,3,3-trifluoropropyl)piperazin-1-yl)phenyl)-2-fluorobenzamide (Compound 26)

To a suspension of N-(4-chloro-2-(4-(3,3,3-trifluoropropyl)piperazin-1-yl)phenyl)-4-cyano-2-fluorobenzamide (0.1 g, 0.220 mmol) and cobalt(II) chloride hexahydrate (0.105 g, 0.440 mmol) in methanol (2 mL), was added NaBH₄ (0.083 g, 2.199 mmol) at 0° C. After stirring for 1 h at room temperature, the reaction was concentrated under reduced pressure. The residue was quenched with 2M HCl and washed with ether. The aqueous layer was basified with 1M NaOH, and then extracted with CH₂Cl₂. The organic layer was dried over MgSO₂ and concentrated under reduced pressure. The resulting residue was purified by column chromatography to give the title compound (0.056 g, 55.5%). LC/MS m/z=459.2 [M+H]⁺;

### Example 1.186

### Preparation of 4-(Aminomethyl)-N-(4-chloro-2-(4-(3,3,3-trifluoropropyl)piperazin-1-yl)phenyl)-2,3-difluorobenzamide (Compound 48)

### Step A: Preparation of ethyl 4-(bromomethyl)-2,3-difluorobenzoate

To a solution of ethyl 2,3-difluoro-4-methylbenzoate (1.0 g, 5.00 mmol) in CCl₄ (20 mL), was added benzoic peroxyanhydride (0.121 g, 0.500 mmol) followed by N-bromosuccinimide (1.06 g, 5.99 mmol). The reaction was heated at 90° C. for 3 h. The reaction was cooled to room temperature and the solid material was filtered off. The filtrate was concentrated under reduced pressure and the resulting residue was used for the next step without further purification.

### Step B: Preparation of 4-((tert-butoxycarbonylamino)methyl)-2,3-difluorobenzoic acid

To a solution of ethyl 4-(bromomethyl)-2,3-difluorobenzoate (3.0 g, 10.75 mmol) in CH₂Cl₂ (5 mL), was added potassium 1,3-dioxoisoindolin-2-ide (1.991 g, 10.75 mmol) at ambient temperature. The reaction was stirred at room temperature, washed with water, and purified with silica gel to give an intermediate. The intermediate was dissolved in ethanol (50 mL) and hydrazine (0.689 g, 21.50 mmol) was added. After stirring for 2 h at 80° C., the reaction was concentrated under reduced pressure and extracted with ethyl acetate. The ethyl acetate layer was dried over MgSO₄ and concentrated under reduced pressure. The resulting residue was dissolved in CH₂Cl₂ (10 mL) and di-tert-butyl dicarbonate (2.58 g, 11.82 mmol) and DIEA (1.528 g, 11.82 mmol) was added. After stirring for 2 h at room temperature, the reaction was washed with water, dried over MgSO₄, and then concentrated under reduced pressure. The resulting residue was dissolved in THF (10 mL) and a solution of LiOH (2 g) in H₂O (10 mL). After stirring for 5 h, the volume of the reaction mixture was reduced to 10 mL under reduced pressure and acidified with 2M HCl to pH=5. The resulting solid was filtered, washed with water, and dried under reduced pressure to give the title compound (1.38 g, 45%). ¹H NMR (400 MHz, DMSO-d₆) δ ppm 1.42 (s, 9H), 4.27 (d, J=4.2 Hz, 2H), 7.21 (m, 2H), 7.52 (m, 11H), 7.68 (m, 1H), 12.1 (br, 1H).

### Step C: Preparation of 4-(Aminomethyl)-N-(4-chloro-2-(4-(3,3,3-trifluoropropyl)piperazin-1-yl)phenyl)-2,3-difluorobenzamide (Compound 48)

To a solution of 4-chloro-2-(4-(3,3,3-trifluoropropyl)piperazin-1-yl)aniline (1.0 g, 3.25 mmol) in DMF (2 mL), were added 4-((tert-butoxycarbonylamino)methyl)-2,3-difluorobenzoic acid (0.933 g, 3.25 mmol), HATU (1.236 g, 3.25 mmol), and DIEA (0.420 g, 3.25 mmol) at ambient temperature. After stirring for 12 h, the reaction was extracted with ethyl acetate, dried over MgSO₄, and concentrated under reduced pressure. The resulting residue was treated with 4 M HCl in dioxane (2 mL) and stirred for 5 h. The reaction was concentrated under reduced pressure. The residue was poured into water and 1 M NaOH was added to adjust the pH to 3. The reaction was extracted with ethyl acetate, dried over MgSO₄, and concentrated under reduced pressure to give the title compound (1.3 g, 85%). LC/MS m/z=477.5 [M+H]⁺; ¹H NMR (400 MHz, DMSO-d₆) δ ppm 2.12˜2.32 (bs, 2H), 2.71˜2.43 (bs, 10H), 3.31˜3.52 (bs, 2H), 3.85 (s, 2H), 7.25 (m, 1H), 7.32 (s, 1H), 7.51 (m, 1H), 7.75 (m, 1H), 8.23 (d, J=8.7 Hz, 1H), 9.75 (d, J=8.4 Hz, 1H).

### Example 1.187

### Preparation of N-(4-Chloro-2-(4-(3,3,3-trifluoropropyl)piperazin-1-yl)phenyl)-2-fluoro-4-(piperazin-1-ylmethyl)benzamide (Compound 49)

To a solution of Intermediate 2 (30 mg, 0.057 mmol) in DMF (1 mL), was added piperazine (9.89 mg, 0.115 mmol). The reaction was stirred at 45° C. for 2 h. The mixture was purified by HPLC to give the title compound (12 mg, 39.6%). LC/MS m/z=528.8 [M+H]⁺.

### Examples 1.188 to 1.202

The following compounds were prepared using the disclosed intermediates and a method similar to the one described in Example 1.187.

| | | | Product |
|---|---|---|---|
| Example | Cmpd | | (Mass Observed |
| No. | No. | Intermediates Used | LC/MS m/z) |
| 1.188 | 50 | Intermediate 2 and dioxothiomorpholine | 577.5 [M + H]⁺ |
| 1.189 | 51 | Intermediate 2 and (S)-pyrrolidin-3-ol | 529.7 [M + H]⁺ |
| 1.190 | 52 | Intermediate 2 and 3- | 526.5 [M + H]⁺ |
| | | (methylamino)propanenitrile | |
| 1.191 | 53 | Intermediate 2 and N,N-dimethylpiperidin-4- | 570.75 [M + H]⁺ |
| | | amine | |
| 1.192 | 54 | Intermediate 2 and piperidin-4-ylmethanol | 557.7 [M + H]⁺ |
| 1.193 | 55 | Intermediate 2 and 2-aminoacetamide | 516.6 [M + H]⁺ |
| | | hydrochloride | |
| 1.194 | 56 | Intermediate 2 and (S)-pyrrolidine-2- | 556.6 [M + H]⁺ |
| | | carboxamide | |
| 1.195 | 57 | Intermediate 2 and 2-(methylamino)ethanol | 517.6 [M + H]⁺ |
| 1.196 | 58 | Intermediate 2 and 2-(piperidin-2-yl)ethanol | 571.5 [M + H]⁺ |
| 1.197 | 59 | Intermediate 2 and piperazine-1-carboxamide | 571.5 [M + H]⁺ |
| 1.198 | 60 | Intermediate 2 and piperazin-2-one | 542.6 [M + H]⁺ |
| 1.199 | 61 | Intermediate 2 and (1-methylpiperidin-4- | 570.7 [M + H]⁺ |
| | | yl)methanamine | |
| 1.200 | 62 | Intermediate 2 and 2-aminoethanol | 503.4 [M + H]⁺ |
| 1.201 | 63 | Intermediate 2 and 3-(1H-imidazol-1- | 567.6 [M + H]⁺ |
| | | yl)propan-1-amine | |
| 1.202 | 64 | Intermediate 2 and 4-methylpyridin-3-amine | 550.6 [M + H]⁺ |

### Examples 1.203 to 1.212

The following compounds were prepared using the disclosed intermediates and a method similar to the one described in Example 1.187 followed by deprotection in a similar manner as described in Example 1.12.

| | | | Product |
|---|---|---|---|
| Example | Cmpd | | (Mass Observed |
| No. | No. | Intermediates Used | LC/MS m/z) |
| 1.203 | 84 | Intermediate 2 and (S)-tert-butyl pyrrolidin-3- | 528.5 [M + H]⁺ |
| | | ylcarbamate | |
| 1.204 | 85 | Intermediate 2 and (R)-tert-butyl pyrrolidin-3- | 528.6 [M + H]⁺ |
| | | ylcarbamate | |
| 1.205 | 86 | Intermediate 2 and (R)-tert-butyl 2- | 542.6 [M + H]⁺ |
| | | (aminomethyl)pyrrolidine-1-carboxylate | |
| 1.206 | 87 | Intermediate 2 and (S)-tert-butyl 3- | 528.7 [M + H]⁺ |
| | | aminopyrrolidine-1-carboxylate | |
| 1.207 | 88 | Intermediate 2 and (R)-tert-butyl 3- | 542.5 [M + H]⁺ |
| | | methylpiperazine-1-carboxylate | |
| 1.208 | 89 | Intermediate 2 and (S)-tert-butyl piperidin-3- | 542.6 [M + H]⁺ |
| | | ylcarbamate | |
| 1.209 | 90 | Intermediate 2 and (R)-tert-butyl piperidin-3- | 542.6 [M + H]⁺ |
| | | ylcarbamate | |
| 1.210 | 91 | Intermediate 2 and (S)-tert-butyl 3- | 542.4 [M + H]⁺ |
| | | aminopiperidine-1-carboxylate | |
| 1.211 | 92 | Intermediate 2 and tert-butyl piperidin-4- | 542.6 [M + H]⁺ |
| | | ylcarbamate | |
| 1.212 | 93 | Intermediate 2 and tert-butyl 4- | 542.3 [M + H]⁺ |
| | | aminopiperidine-1-carboxylate | |

### Example 1.213

### Preparation of N-(4-Chloro-2-(4-(3,3,3-trifluoropropyl)piperazin-1-yl)phenyl)-2,3-difluoro-4-(4-(hydroxymethyl)piperidin-1-yl)methyl)benzamide (Compound 165)

To a solution of Intermediate 1 (20 mg, 0.037 mmol) in DMF (1 mL), was added piperidin-4-ylmethanol (4.25 mg, 0.037 mmol) and then stirring at 45° C. for 2 h. The mixture was purified by HPLC to give the title compound (14 mg, 65.8%). LC/MS m/z=575.6 [M+H]⁺.

### Examples 1.214 to 1.217

The following compounds were prepared using the disclosed intermediates and a method similar to the one described in Example 1.213.

| | | | Product |
|---|---|---|---|
| Example | Cmpd | | (Mass Observed |
| No. | No. | Intermediates Used | LC/MS m/z) |
| 1.214 | 166 | Intermediate 1 and | 561.4 [M + H]⁺ |
| | | piperidin-4-ol | |
| 1.215 | 167 | Intermediate 1 and | 561.5 [M + H]⁺ |
| | | piperazin-2-one | |
| 1.216 | 168 | Intermediate 1 and | 595.5 [M + H]⁺ |
| | | dioxothiomorpholine | |
| 1.217 | 169 | Intermediate 1 and (S)- | 574.6 [M + H]⁺ |
| | | pyrrolidine-2-carboxamide | |

### Example 1.218

### Preparation of (S)-4-(1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) as the Di-Hydrochloride Salt

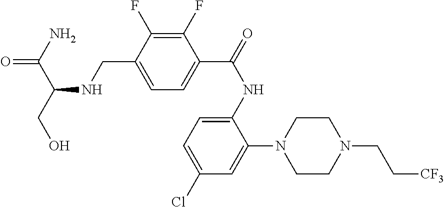 [SMILES: NC(=O)[C@H](CO)NCc1ccc(C(=O)Nc2ccc(Cl)cc2N2CCN(CCC(F)(F)F)CC2)c(F)c1F]

To a mixture of Intermediate 1 (15.1 g, 30.4 mmol), (S)-2-amino-3-hydroxypropanamide hydrochloride (5.99 g, 42.6 mmol), and DIEA (15.94 mL, 91 mmol) was added DMF (60 mL). The reaction was heated at 90° C. for 3 h. The mixture was extracted with H₂O/EtOAc (2×200 mL). The organic layer was back extracted once with H₂O (500 mL). The organic layer was dried and concentrated to give a solid. The solid was re-suspended/dissolved in ACN (150 mL). HCl (4 M in dioxane, 22.82 mL, 91 mmol) was added dropwise to the solution. The reaction was stirred at room temperature for 2 h. The precipitate was filtered, washed with ACN, and dried under reduced pressure in an oven (50° C.) to give a solid (16.9 g). The solid was extracted again under basic conditions with EtOAc/aqueous Na₂CO₃ (2×200 mL). The combined organics were de-colored by activated carbon (˜1 g), dried, filtered, and concentrated. The residue was dissolved completely in ACN (800 mL). Then HCl (4 M in dioxane, 22.82 mL, 91 mmol) was slowly added via an addition funnel to provide a precipitate. Upon complete addition of HCl, the reaction was stirred at room temperature for 2 h. After this time, the precipitate was filtered, washed with ACN, and dried under reduced pressure in an oven (50° C.) over the weekend to give the dihydrochloride salt of the title compound (13.5 g, 69.0%) as a white solid. LC/MS m/z=564.4 [M+H]⁺; ¹H NMR (400 MHz, DMSO-d₆) δ ppm 2.88-3.13 (m, 2H), 3.38-3.57 (m, 8H), 3.57-3.77 (m, 2H), 3.82-4.01 (m, 3H), 4.25-4.42 (m, 2H), 5.61 (bs, 1H), 7.31 (d, J=8.65 Hz, 1H), 7.36 (s, 1H), 7.57-7.81 (m, 3H), 8.02 (s, 1H), 8.15 (d, J=7.63 Hz, 1H), 9.45 (bs, 1H), 9.66 (bs, 1H), 9.83 (d, J=3.05 Hz, 1H), 11.74 (bs, 1H).

A sample was analyzed by ultra performance liquid chromatography (i.e., UPLC) and was observed to contain 86.6% by weight of the free base (theoretical amount of free base is 88.5% for the dihydrochloride and 93.9% for monohydrochloride) indicating dihydrochloride stoichiometry.

### Examples 1.219 to 1.232

The following compounds were prepared using the disclosed intermediates and a method similar to the one described in Example 1.213.

| | | | Product |
|---|---|---|---|
| Example | Cmpd | | (Mass Observed |
| No. | No. | Intermediates Used | LC/MS m/z) |
| 1.219 | 171 | Intermediate 1 and tetrahydrodioxothiophen-3- | 595.6 [M + H]⁺ |
| | | amine | |
| 1.220 | 172 | Intermediate 1 and 2-(methylamino)ethanol | 535.2 [M + H]⁺ |
| 1.221 | 173 | Intermediate 1 and 2-(piperidin-2-yl)ethanol | 589.6 [M + H]⁺ |
| 1.222 | 174 | Intermediate 1 and (R)-pyrrolidin-2-ylmethanol | 561.6 [M + H]⁺ |
| 1.223 | 175 | Intermediate 1 and 2-(piperazin-1-yl)ethanol | 590.6 [M + H]⁺ |
| 1.224 | 176 | Intermediate 1 and (1S,2R)-2- | 575.7 [M + H]⁺ |
| | | aminocyclohexanol | |
| 1.225 | 177 | Intermediate 1 and (S)-pyrrolidin-3-ol | 547.5 [M + H]⁺ |
| 1.226 | 178 | Intermediate 1 and 2,2′-azanediyldiethanol | 565.5 [M + H]⁺ |
| 1.227 | 179 | Intermediate 1 and azetidin-3-ol | 533.5 [M + H]⁺ |
| 1.228 | 180 | Intermediate 1 and morpholin-2-ylmethanol | 577.5 [M + H]⁺ |
| 1.229 | 181 | Intermediate 1 and (R)-piperidin-3-ol | 561.4 [M + H]⁺ |
| 1.230 | 182 | Intermediate 1 and (S)-piperidin-3-ol | 561.5 [M + H]⁺ |
| 1.231 | 183 | Intermediate 1 and 2-(piperidin-4-yl)ethanol | 589.6 [M + H]⁺ |
| 1.232 | 184 | Intermediate 1 and piperazine | 546.4 [M + H]⁺ |

### Example 1.233

### Preparation of 4-Amino-N-(4-chloro-2-(4-(3,3,3-trifluoropropyl)piperazin-1-yl)phenyl)-2,3-difluorobenzamide (Compound 236)

A mixture of 4-amino-2,3-difluorobenzoic acid (45.0 mg, 0.260 mmol), 4-chloro-2-(4-(3,3,3-trifluoropropyl)piperazin-1-yl)aniline (80 mg, 0.260 mmol), HATU (148 mg, 0.390 mmol) and TEA (0.109 mL, 0.780 mmol) in DMF (1 mL) was heated to 50° C. for 18 h in a 5 mL sealed scintillation vial. The mixture was purified by preparative HPLC (10%-95% MeCN/H₂O) to give the product as white solid. LC/MS m/z=463.4 [M+H]⁺; ¹H NMR (400 MHz, CD₃OD) δ ppm 2.92 (m, 2H), 3.31 (m, 4H), 3.68-3.53 (m, 6H), 6.78 (dd, J_{I}=J₂=7.9 Hz, 1H), 7.32 (d, J=8.4 Hz, 1H), 7.42 (s, 1H), 7.66 (dd, J₁=J₂=9.1 Hz, 1H), 8.44 (d, J=8.6 Hz, 1H).

### Examples 1.234 to 1.236

The following compounds were prepared using the disclosed intermediates and a method similar to the one described in Example 1.233.

| | | | Product |
|---|---|---|---|
| Example | Cmpd | | (Mass Observed |
| No. | No. | Intermediates Used | LC/MS m/z) |
| 1.234 | 205¹ | 4-chloro-2-(4-(3,3,3-trifluoropropyl)piperazin-1- | 479.4 [M + H]⁺ |
| | | yl)aniline and 4-amino-3-chloro-2-fluorobenzoic | |
| | | acid | |
| 1.235 | 193 | 4-chloro-2-(4-(3,3,3-trifluoropropyl)piperazin-1- | 445.5 [M + H]⁺ |
| | | yl)aniline and 4-amino-2-fluorobenzoic acid | |
| 1.236 | 194 | 4-chloro-2-(4-(3,3,3-trifluoropropyl)piperazin-1- | 479.3 [M + H]⁺ |
| | | yl)aniline and 4-amino-5-chloro-2-fluorobenzoic | |
| | | acid | |

¹Compound 205: ¹H NMR (400 MHz, CD₃OD) δ ppm 2.82 (m, 2H), 3.21 (m, 4H), 3.48 (m, 6H), 6.73 (dd, J = 8.9, 1.0 Hz, 1H), 7.24 (dd, J = 8.7, 2.2 Hz, 1H), 7.35 (d, J = 2.4 Hz, 1H), 7.72 (dd, J = 8.8 Hz, 1H), 8.35 (d, J = 8.5 Hz, 1H).

### Example 1.237

### Preparation of (S)—N-(4-Chloro-2-(4-(3,3,3-trifluoropropyl)piperazin-1-yl)phenyl)-2,3-difluoro-4-((3-(hydroxymethyl)piperidin-1-yl)methyl)benzamide (Compound 251)

Intermediate 1 (10 mg, 0.020 mmol), (S)-piperidin-3-ylmethanol hydrochloride (3.67 mg, 0.024 mmol), and DIEA (10.56 μL, 0.060 mmol) were added to a vial with DMF (0.3 mL). The reaction was stirred with heating to 80° C. for 2 h. After this time, the reaction was fairly complete. The reaction mixture was purified by preparative HPLC (5-70% ACN/H₂O, 30 min), and then converted to its corresponding HCl salt by re-dissolving the material in ACN (0.2 mL) and H₂O (0.8 mL), and adding HCl (4 eq.). The reaction was stirred at room temperature for 1 h. Then the reaction was frozen and lyophilized to yield the title compound (13.0 mg, 99%). LC/MS m/z=575.4 [M+H]⁺; ¹H NMR (400 MHz, DMSO-d₆) δ ppm 1.02-1.24 (m, 1H), 1.67 (d, J=13.99 Hz, 1H), 1.73-1.90 (m, 2H), 1.92-2.09 (m, 1H), 2.62-2.85 (m, 1H), 2.85-3.06 (m, 4H), 3.06-3.31 (m, 9H), 3.51-3.83 (m, 5H), 4.46 (s, 2H), 7.29 (d, J=8.39 Hz, 1H), 7.35 (s, 1H), 7.59-7.83 (m, 2H), 8.12 (d, J=7.63 Hz, 1H), 9.84 (s, 1H), 10.46-10.68 (m, 1H).

### Example 1.238

### Preparation of (R)—N-(4-Chloro-2-(4-(3,3,3-trifluoropropyl)piperazin-1-yl)phenyl)-2,3-difluoro-4-((3-(hydroxymethyl)piperidin-1-yl)methyl)benzamide (Compound 252)

From Intermediate 1 and (R)-piperidin-3-ylmethanol hydrochloride, using a similar method to the one described in Example 1.237, the title compound was obtained. LC/MS m/z=575.4 [M+H]⁺; ¹H NMR (400 MHz, DMSO-d₆) δ ppm 1.04-1.22 (m, J=12.21 Hz, 1H), 1.67 (d, J=10.68 Hz, 1H), 1.73-1.90 (m, 2H), 1.93-2.08 (m, 1H), 2.64-2.83 (m, 1H), 2.82-3.06 (m, 4H), 3.03-3.31 (m, 9H), 3.53-3.96 (m, 5H), 4.46 (s, 2H), 7.29 (d, J=8.39 Hz, 1H), 7.34 (s, 1H), 7.63-7.79 (m, 2H), 8.04-8.20 (m, 1H), 9.84 (s, 1H), 10.43-10.71 (m, 1H).

### Example 1.239

### Preparation of 1-(4-(4-Chloro-2-(4-(3,3,3-trifluoropropyl)piperazin-1-yl)phenylcarbamoyl)-2,3-difluorobenzyl)piperazine-2-carboxamide (Compound 239)

To a solution of ethyl 4-(tert-butoxycarbonyl)-1-(4-(4-chloro-2-(4-(3,3,3-trifluoropropyl)piperazin-1-yl)phenylcarbamoyl)-2,3-difluorobenzyl)piperazine-2-carboxylic acid (30 mg, 0.043 mmol) in DMF (2 mL), was added ammonia (10 M in methanol, 43 μL, 0.043 mmol). After stirring for 30 min, the reaction was treated with 2-(3H-[1,2,3]triazolo[4,5-b]pyridin-3-yl)-1,1,3,3-tetramethylisouronium hexafluorophosphate(V) (16.53 mg, 0.043 mmol). The reaction was stirred for 1 h at 50° C. The reaction was extracted with ethyl acetate and concentrated under reduced pressure. The resulting residue was purified by column chromatography. The purified compound was dissolved in acetonitrile (2 mL), and then 4.0 M HCl in dioxane (0.5 mL) was added. After stirring for 30 min, the precipitate was filtered off and dried to give the title compound (12 mg, 46.9%). LC/MS m/z=589.7 [M+H]⁺.

### Examples 1.240 to 1.242

The following compounds were prepared using the disclosed intermediates and a method similar to the one described in Example 1.239.

| | | | Product |
|---|---|---|---|
| Example | Cmpd | | (Mass Observed |
| No. | No. | Intermediates Used | LC/MS m/z) |
| 1.240 | 240 | 4-(tert-butoxycarbonyl)-1-(4-(4-chloro-2-(4- | 604.51 [M + H]⁺ |
| | | (3,3,3-trifluoropropyl)piperazin-1- | |
| | | yl)phenylcarbamoyl)-2,3- | |
| | | difluorobenzyl)piperazine-2-carboxylic acid and | |
| | | methylamine | |
| 1.241 | 241 | 4-(tert-butoxycarbonyl)-1-(4-(4-chloro-2-(4- | 634.52 [M + H]⁺ |
| | | (3,3,3-trifluoropropyl)piperazin-1- | |
| | | yl)phenylcarbamoyl)-2,3- | |
| | | difluorobenzyl)piperazine-2-carboxylic acid with | |
| | | 2-aminoethanol | |
| 1.242 | 242 | 4-(tert-butoxycarbonyl)-1-(4-(4-chloro-2-(4- | 648.51 [M + H]⁺ |
| | | (3,3,3-trifluoropropyl)piperazin-1- | |
| | | yl)phenylcarbamoyl)-2,3- | |
| | | difluorobenzyl)piperazine-2-carboxylic acid with | |
| | | (S)-2-aminopropan-1-ol | |

### Example 1.243

### Preparation of (S)-3-Amino-2-(4-(4-chloro-2-(4-(3,3,3-trifluoropropyl)piperazin-1-yl)phenylcarbamoyl)-2,3-difluorobenzylamino)-3-oxopropyl dihydrogenphosphate (Compound 249)

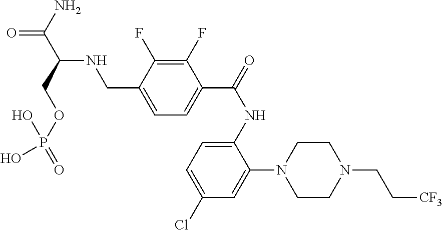 [SMILES: NC(=O)[C@H](COP(=O)(O)O)NCc1ccc(C(=O)Nc2ccc(Cl)cc2N2CCN(CCC(F)(F)F)CC2)c(F)c1F]

To a solution of (S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (1.0 g, 1.773 mmol) in THF (3.00 mL) and H₂O (3.00 mL), were added di-tert-butyl dicarbonate (0.464 g, 2.128 mmol) and K₂CO₃ (0.245 g, 1.773 mmol) to the reaction at room temperature. After stirring for 12 h, the reaction was poured into water, extracted with ethyl acetate. The organics were purified by column chromatography to give (S)-tert-butyl 1-amino-3-hydroxy-1-oxopropan-2-yl(4-(4-chloro-2-(4-(3,3,3-trifluoropropyl)piperazin-1-yl)phenylcarbamoyl)-2,3-difluorobenzyl)carbamate (0.56 g). The above material (300 mg, 0.452 mmol) was dissolved in THF (2 mL) and diallyl diisopropylphosphoramidite (222 mg, 0.904 mmol) was added followed by 1H-tetrazole (95 mg, 1.355 mmol) at room temperature. After stirring for 2 h, the reaction was treated with 2-hydroperoxy-2-methylpropane (81 mg, 0.904 mmol). After stirring for 2 h, the reaction was extracted with ethyl acetate, dried over MgSO₄, concentrated and dried under reduced pressure. The residue was purified by column chromatography to give (S)-tert-butyl 1-amino-3-(bis(allyloxy)phosphoryloxy)-1-oxopropan-2-yl(4-(4-chloro-2-(4-(3,3,3-trifluoropropyl)piperazin-1-yl)phenylcarbamoyl)-2,3-difluorobenzyl)carbamate (123 mg), which was dissolved in THF (1 mL), and Pd(PPh₃)₄ (5.61 mg, 4.85 μmol) and pyrrolidine (51.8 mg, 0.728 mmol) were added. After stirring for 1 h, the reaction was filtered and concentrated under reduced pressure. The residue was treated with 2 eq. TFA and stirred for 10 h. The mixture was concentrated under reduced pressure and treated with 1 M aq. NaOH (1 mL) in acetonitrile (0.5 mL). The resulting solution was purified by C-18 reverse phase column chromatography to give the title compound (89 mg). LC/MS m/z=644.5 [M+H]⁺.

### Examples 1.244 to 1.249

The following compounds were prepared using the disclosed intermediates and a method similar to the one described in Example 1.213.

| | | | Product |
|---|---|---|---|
| Example | Cmpd | | (Mass Observed |
| No. | No. | Intermediates Used | LC/MS m/z) |
| 1.244 | 253 | Intermediate 1 and (S)-pyrrolidin-2-ylmethanol | 561.45 [M + H]⁺ |
| 1.245 | 254 | Intermediate 1 and (S)-pyrrolidin-3-ylmethanol | 561.46 [M + H]⁺ |
| 1.246 | 255 | Intermediate 1 and (1R,2S)-2- | 588.46 [M + H]⁺ |
| | | aminocyclopentanecarboxamide | |
| 1.247 | 237 | Intermediate 1 and (1R,2S)-2- | 602.49 [M + H]⁺ |
| | | aminocyclohexanecarboxamide | |
| 1.248 | 47 | Intermediate 1 and (1S,2R)-2- | 561.42 [M + H]⁺ |
| | | aminocyclopentanol | |
| 1.249 | 482 | Intermediate 1 and (1R,2R)-2- | 561.42 [M + H]⁺ |
| | | aminocyclopentanol | |

### Examples 1.250 to 1.255

The following compounds were prepared using the disclosed intermediates and a method similar to the one described in Example 1.126.

| | | | Product |
|---|---|---|---|
| Example | Cmpd | | (Mass Observed |
| No. | No. | Intermediates Used | LC/MS m/z) |
| 1.250 | 35 | 4-chloro-2-(4-(3,3,3-trifluoropropyl)piperazin-1- | 455.1 [M + H]⁺ |
| | | yl)aniline and 4-(dimethylamino)benzoic acid | |
| 1.251 | 38 | 4-chloro-2-(4-(3,3,3-trifluoropropyl)piperazin-1- | 478.1 [M + H]⁺ |
| | | yl)aniline and 4-(1H-imidazol-1-yl)benzoic acid | |
| 1.252 | 39 | 4-chloro-2-(4-(3,3,3-trifluoropropyl)piperazin-1- | 511.2 [M + H]⁺ |
| | | yl)aniline and 4-(morpholinomethyl)benzoic acid | |
| 1.253 | 40 | 4-chloro-2-(4-(3,3,3-trifluoropropyl)piperazin-1- | 510.2 [M + H]⁺ |
| | | yl)aniline and 4-(4-methylpiperazin-1-yl)benzoic | |
| | | acid | |
| 1.254 | 41 | 4-chloro-2-(4-(3,3,3-trifluoropropyl)piperazin-1- | 481.1 [M + H]⁺ |
| | | yl)aniline and 4-(pyrrolidin-1-yl)benzoic acid | |
| 1.255 | 42 | 4-chloro-2-(4-(3,3,3-trifluoropropyl)piperazin-1- | 497.1 [M + H]⁺ |
| | | yl)aniline and 4-morpholinobenzoic acid | |

### Example 1.256

### Preparation of N-(4-(4-Chloro-2-(4-(3,3,3-trifluoropropyl)piperazin-1-yl)phenylcarbamoyl)-2,3-difluorobenzyl)piperidine-4-carboxamide (Compound 261)

### Step A: Preparation of Ethyl 4-(Bromomethyl)-2,3-difluorobenzoate

To a solution of ethyl 2,3-difluoro-4-methylbenzoate (1.0 g, 5.00 mmol) in CCl₄ (20 mL), was added benzoic peroxyanhydride (0.121 g, 0.500 mmol) followed by 1-brompyrrolidine-2,5-dione (1.06 g, 5.99 mmol). The reaction was heated at 90° C. for 3 h. The reaction was cooled to room temperature and filtered off the solid material. The filtrate was concentrated under reduced pressure to give the title compound without further purification.

### Step B: Preparation of 4-((tert-Butoxycarbonylamino)methyl)-2,3-difluorobenzoic Acid

To a solution of ethyl 4-(bromomethyl)-2,3-difluorobenzoate (3.0 g, 10.75 mmol) in DCM (5 mL), was potassium 1,3-dioxoisoindolin-2-ide (1.991 g, 10.75 mmol) added at room temperature. After stirring for 2 h, the reaction was washed with water, dried and MgSO₄, and concentrated under reduced pressure. The residue was dissolved in ethanol (50 mL) and hydrazine (0.689 g, 21.50 mmol) was added. After stirring for 2 h at 80° C., the reaction was concentrated under reduced pressure and extracted with ethyl acetate. The organic layer was dried over MgSO₄ and concentrated under reduced pressure. The residue was dissolved in DCM (10 mL) and di-tert-butyl dicarbonate (2.58 g, 11.82 mmol) and DIEA (1.528 g, 11.82 mmol) were added at room temperature. After stirring for 2 h, the reaction was washed with water, dried over MgSO₄, and concentrated under reduced pressure to give ethyl 4-((tert-butoxycarbonylamino)methyl)-2,3-difluorobenzoate, which was dissolved in THF (5 mL) and a solution of LiOH (0.95 g) in H₂O (5 mL) was added. After stirring for 5 h, the reaction was reduced the volume to 10 mL under reduced pressure and the pH was adjusted to 5 and 2.0 M HCl. The resulting solid was filtered, washed with water, and dried under reduced pressure to give the title compound (1.98 g, 64.5%). LCMS m/z=316.3 [M+H]⁺. ¹H NMR (400 MHz, DMSO-d₆) δ ppm 1.42 (s, 9H), 4.27 (d, J=4.2 Hz, 2H), 7.21 (m, 2H), 7.52 (m, 1H), 7.68 (m, 1H), 12.1 (br, 1H).

### Step C: Preparation of tert-Butyl 4-(4-(4-Chloro-2-(4-(3,3,3-trifluoropropyl)piperazin-1-yl)phenylcarbamoyl)-2,3-difluorobenzylcarbamoyl)piperidine-1-carboxylate (Compound 243) and 4-(Aminomethyl)-N-(4-chloro-2-(4-(3,3,3-trifluoropropyl)piperazin-1-yl)phenyl)-2,3-difluorobenzamide (Intermediate 3)

To a solution of 4-chloro-2-(4-(3,3,3-trifluoropropyl)piperazin-1-yl)aniline (3.21 g, 10.44 mmol) in DMF (12 mL), was added 4-((tert-butoxycarbonylamino)methyl)-2,3-difluorobenzoic acid (3.0 g, 10.44 mmol), HATU (3.97 g, 10.44 mmol), and DIEA (1.35 g, 10.44 mmol) at ambient temperature. After stirring for 12 h, the reaction was extracted with ethyl acetate, dried over MgSO₄, and concentrated under reduced pressure to provide Compound 243. The resulting compound was treated with 4.0 M HCl in dioxane (2 mL), stirred for 12 h, and the reaction was concentrated under reduced pressure. The residue was dissolved in ethyl acetate and washed with aq. NaHCO₃, dried over MgSO₄, and concentrated under reduced pressure to give the title compound (3.54 g, 71.1%). LCMS m/z=477.5 [M+H]⁺. ¹H NMR (400 MHz, DMSO-d₆) δ ppm 2.12-2.32 (br, 2H), 2.71-2.43 (br, 10H), 3.31-3.52 (br, 2H), 3.85 (s, 2H), 7.25 (m, 1H), 7.32 (s, 1H), 7.51 (m, 1H), 7.75 (m, 1H), 8.23 (d, J=8.7 Hz, 1H), 9.75 (d, J=8.4 Hz, 1H).

### Step D: Preparation of di HCl Salt of N-(4-(4-Chloro-2-(4-(3,3,3-trifluoropropyl)piperazin-1-yl)phenylcarbamoyl)-2,3-difluorobenzyl)piperidine-4-carboxamide (Compound 261)

To a solution of 4-(aminomethyl)-N-(4-chloro-2-(4-(3,3,3-trifluoropropyl)piperazin-1-yl)phenyl)-2,3-difluorobenzamide (30 mg, 0.063 mmol) in DMF (1 mL), was added 1-(tert-butoxycarbonyl)piperidine-4-carboxylic acid (14.4 mg, 0.063 mmol) followed by HATU (23.9 mg, 0.063 mmol) and DIEA (8.3 mg, 0.063 mmol) at room temperature. The reaction was stirred at 60° C. for 1 h. The mixture was purified by HPLC and concentrated under reduced pressure. The resulting material was treated with 4.0 M HCl in dioxane (1 mL) and concentrated under reduced pressure to give the title compound (17 mg, 46.8%). LCMS m/z=588.5[M+H]⁺.

### Examples 1.257 to 1.264

The following compounds were prepared using the disclosed intermediates and a method similar to the one described in Example 1.256.

| | | | Product |
|---|---|---|---|
| Example | Cmpd | | (Mass Observed |
| No. | No. | Intermediates Used | LC/MS m/z) |
| 1.257 | 444 | Intermediate 3 and (1R,2S)-2-(tert- | N.D.¹ |
| | | butoxycarbonylamino)cyclohexanecarboxylic | |
| | | acid | |
| | 262 | Deprotection | 602.6 [M + H]⁺ |
| 1.258 | 445 | Intermediate 3 and (S)-1-(tert- | N.D.¹ |
| | | butoxycarbonyl)piperidine-3-carboxylic acid | |
| | 263 | Deprotection | 588.5 [M + H]⁺ |
| 1.259 | 446 | Intermediate 3 and 4-(tert- | N.D.¹ |
| | | butoxycarbonyl)morpholine-2-carboxylic acid | |
| | 264 | Deprotection | 590.5 [M + H]⁺ |
| 1.260 | 447 | Intermediate 3 and 3-(tert- | N.D.¹ |
| | | butoxycarbonylamino)propanoic acid | |
| | 265 | Deprotection | 548.5 [M + H]⁺ |
| 1.261 | 448 | Intermediate 3 and 2-(tert- | N.D.¹ |
| | | butoxycarbonylamino)acetic acid | |
| | 266 | Deprotection | 534.4 [M + H]⁺ |
| 1.262 | 323 | Intermediate 3 and (2S,4R)-1-(tert- | N.D.¹ |
| | | butoxycarbonyl)-4-hydroxypyrrolidine-2- | |
| | | carboxylic acid | |
| | 267 | Deprotection | 590.5 [M + H]⁺ |
| 1.263 | 450 | Intermediate 3 and (S)-2-(tert- | N.D.¹ |
| | | butoxycarbonylamino)propanoic acid | |
| | 268 | Deprotection | 548.5 [M + H]⁺ |
| 1.264 | 451 | Intermediate 3 and (S)-2-(tert- | N.D.¹ |
| | | butoxycarbonylamino)-3-hydroxypropanoic acid | |
| | 269 | Deprotection | 564.3 [M + H]⁺ |

¹Not Determined

### Example 1.265

### Preparation of (S)-tert-Butyl 2-(4-(4-Chloro-2-(4-(3,3,3-trifluoropropyl)piperazin-1-yl)phenylcarbamoyl)-2,3-difluorobenzylcarbamoyl)morpholine-4-carboxylate (Compound 452) and di HCl Salt of (S)—N-(4-(4-Chloro-2-(4-(3,3,3-trifluoropropyl)piperazin-1-yl)phenylcarbamoyl)-2,3-difluorobenzyl)morpholine-2-carboxamide (Compound 271)

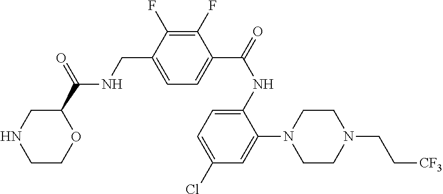 [SMILES: O=C(Nc1ccc(Cl)cc1N1CCN(CCC(F)(F)F)CC1)c1ccc(CNC(=O)[C@@H]2CNCCO2)c(F)c1F]

From Intermediate 3 and (S)-4-(tert-butoxycarbonyl)morpholine-2-carboxylic acid, the title compounds were obtained using a method similar to the one described in Example 1.256.

Compound 271: LCMS m/z=590.5 [M+H]⁺.

### Example 1.266

### Preparation of (R)-tert-butyl 2-(4-(4-chloro-2-(4-(3,3,3-trifluoropropyl)piperazin-1-yl)phenylcarbamoyl)-2,3-difluorobenzylcarbamoyl)morpholine-4-carboxylate (Compound 453) and di HCl Salt of (R)—N-(4-(4-Chloro-2-(4-(3,3,3-trifluoropropyl)piperazin-1-yl)phenylcarbamoyl)-2,3-difluorobenzyl)morpholine-2-carboxamide (Compound 272)

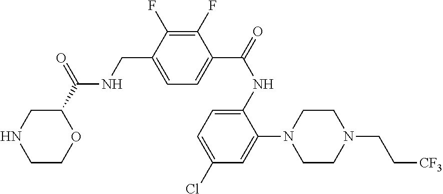 [SMILES: O=C(Nc1ccc(Cl)cc1N1CCN(CCC(F)(F)F)CC1)c1ccc(CNC(=O)[C@H]2CNCCO2)c(F)c1F]

From Intermediate 3 and (R)-4-(tert-butoxycarbonyl)morpholine-2-carboxylic acid, the title compounds were obtained using a method similar to the one described in Example 1.256.

Compound 272: LCMS m/z=590.5 [M+H]⁺.

### Examples 1.267 to 1.273

The following compounds were prepared using the disclosed intermediates and a method similar to the one described in Example 1.256.

| | | | Product |
|---|---|---|---|
| Example | Cmpd | | (Mass Observed |
| No. | No. | Intermediates Used | LC/MS m/z) |
| 1.267 | 454 | Intermediate 3 and (R)-4-(tert- | N.D.¹ |
| | | butoxycarbonyl)thiomorpholine-3-carboxylic | |
| | | acid | |
| | 273 | Deprotection | 606.6 [M + H]⁺ |
| 1.268 | 455 | Intermediate 3 and (S)-1-(tert- | N.D.¹ |
| | | butoxycarbonyl)pyrrolidine-2-carboxylic acid | |
| | 274 | Deprotection | 574.6 [M + H]⁺ |
| 1.269 | 456 | Intermediate 3 and (R)-2-(tert- | N.D.¹ |
| | | butoxycarbonylamino)propanoic acid | |
| | 275 | Deprotection | 548.4 [M + H]⁺ |
| 1.270 | 457 | Intermediate 3 and (2S)-1-(tert- | N.D.¹ |
| | | butoxycarbonyl)-4-hydroxypyrrolidine-2- | |
| | | carboxylic acid | |
| | 276 | Deprotection | 590.5 [M + H]⁺ |
| 1.271 | 458 | Intermediate 3 and 4-(tert- | N.D.¹ |
| | | butoxycarbonylamino)tetrahydro-2H-thiopyran- | |
| | | 4-carboxylic acid | |
| | 284 | Deprotection | 620.6 [M + H]⁺ |
| 1.272 | 459 | Intermediate 3 and 4-tert-butoxy-4-oxobutanoic | N.D.¹ |
| | | acid | |
| | 293 | Deprotection | 577.4 [M + H]⁺ |
| 1.273 | 460 | Intermediate 3 and (R)-4-(tert- | N.D.¹ |
| | | butoxycarbonyl)morpholine-3-carboxylic acid | |
| | 301 | Deprotection | 590.4 [M + H]⁺ |

¹Not Determined

### Example 1.274

### Preparation of N-(4-Chloro-2-(4-(3,3,3-trifluoropropyl)piperazin-1-yl)phenyl)-2,3-difluoro-4-((2-hydroxyacetamido)methyl)benzamide (Compound 270)

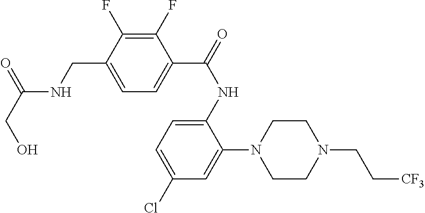 [SMILES: O=C(CO)NCc1ccc(C(=O)Nc2ccc(Cl)cc2N2CCN(CCC(F)(F)F)CC2)c(F)c1F]

To a solution of Intermediate 3 (30 mg, 0.063 mmol) in DMF (1 mL), were added 2-hydroxyacetic acid (4.78 mg, 0.063 mmol), HATU (23.92 mg, 0.063 mmol), and DIEA (8 mg, 0.063 mmol). After stirred for 1 h at 60° C., the reaction was purified by HPLC to give the title compound (15 mg, 44.6%). LCMS m/z=535.4 [M+H]⁺.

### Example 1.275

### Preparation of N-(4-Chloro-2-(4-(3,3,3-trifluoropropyl)piperazin-1-yl)phenyl)-2,3-difluoro-4-((2-morpholinoacetamido)methyl)benzamide (Compound 277)

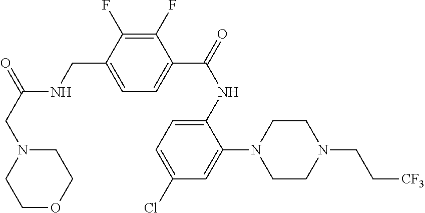 [SMILES: O=C(CN1CCOCC1)NCc1ccc(C(=O)Nc2ccc(Cl)cc2N2CCN(CCC(F)(F)F)CC2)c(F)c1F]

From Intermediate 3 and 2-morpholinoacetic acid, the title compound was obtained using a method similar to the one described in Example 1.274. LCMS m/z=604.4 [M+H]⁺.

### Examples 1.276 to 1.280, 1.282, 1.283, 1.285 to 1.288, 1.290 to 1.294, and 1.299

The following compounds were prepared using the disclosed intermediates and a method similar to the one described in Example 1.274.

| | | | Product |
|---|---|---|---|
| Example | Cmpd | | (Mass Observed |
| No. | No. | Intermediates Used | LC/MS m/z) |
| 1.276 | 278 | Intermediate 3 and 2-(1H-tetrazol-5-yl)acetic acid | 587.6 [M + H]⁺ |
| 1.277 | 279 | Intermediate 3 and 2-(dimethylamino)acetic acid | 562.5 [M + H]⁺ |
| 1.278 | 280 | Intermediate 3 and 3-oxo-2,3-dihydroisoxazole- | 588.4 [M + H]⁺ |
| | | 5-carboxylic acid | |
| 1.279 | 281 | Intermediate 3 and 6-oxo-1,6- | 599.4 [M + H]⁺ |
| | | dihydropyridazine-3-carboxylic acid | |
| 1.280 | 282 | Intermediate 3 and 2,4-dihydroxypyrimidine-5- | 615.3 [M + H]⁺ |
| | | carboxylic acid | |
| 1.282 | 461 | Intermediate 3 and (S)-2-(3-(tert- | N.D. ¹ |
| | | butoxycarbonylamino)-2-oxopyrrolidin-1- | |
| | | yl)acetic acid | |
| | 285 | Deprotection | 617.5 [M + H]⁺ |
| 1.283 | 287 | Intermediate 3 and 6-hydroxynicotinic acid | 598.4 [M + H]⁺ |
| 1.285 | 289 | Intermediate 3 and 2,6-dihydroxyisonicotinic | 614.4 [M + H]⁺ |
| | | acid | |
| 1.286 | 290 | Intermediate 3 and 2,6-dioxo-1,2,3,6- | 615.5 [M + H]⁺ |
| | | tetrahydropyrimidine-4-carboxylic acid | |
| 1.287 | 291 | Intermediate 3 and 5-hydroxy-1-methyl-1H- | 601.2 [M + H]⁺ |
| | | pyrazole-3-carboxylic acid | |
| 1.288 | 292 | Intermediate 3 and 3-(3-hydroxyisoxazol-4- | 616.4 [M + H]⁺ |
| | | yl)propanoic acid | |
| 1.290 | 295 ² | Intermediate 3 and 6-hydroxypicolinic acid | 598.2 [M + H]⁺ |
| 1.291 | 306 | Intermediate 3 and 3-hydroxypropanoic acid | 549.4 [M + H]⁺ |
| 1.292 | 307 | Intermediate 3 and 4- | 603.4 [M + H]⁺ |
| | | hydroxycyclohexanecarboxylic acid | |
| 1.293 | 308 | Intermediate 3 and (R)-3-hydroxypentanoic acid | 577.4 [M + H]⁺ |
| 1.294 | 309 | Intermediate 3 and 3-hydroxypentanoic acid | 577.4 [M + H]⁺ |
| 1.299 | 354 | Intermediate 3 and (1s,4s)-4- | 617.6 [M + H]⁺ |
| | | (hydroxymethyl)cyclohexanecarboxylic acid | |

¹ Not Determined
² Compound 295: ¹H NMR (400 MHz, DMSO-d₆) δ ppm 3.14~3.78 (m, 12H), 4.51 (d, J = 6.1 Hz, 2H), 7.22(m, 4H), 7.61 (m, 1H), 7.96 (m, 2H), 8.13 (m, , 2H), 9.12 (m, 1H), 9.65 (d, J = 4.3 Hz, 1H).

### Example 1.281

### Preparation of N-(4-(4-Chloro-2-(4-(3,3,3-trifluoropropyl)piperazin-1-yl)phenylcarbamoyl)-2,3-difluorobenzyl)-5-oxo-4,5-dihydro-1H-1,2,4-triazole-3-carboxamide (Compound 283)

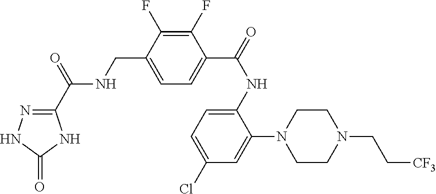 [SMILES: O=C(NCc1ccc(C(=O)Nc2ccc(Cl)cc2N2CCN(CCC(F)(F)F)CC2)c(F)c1F)c1n[nH]c(=O)[nH]1]

From Intermediate 3 and 5-oxo-4,5-dihydro-1H-1,2,4-triazole-3-carboxylic acid, the title compound was obtained using a method similar to the one described in Example 1.274. LCMS m/z=588.3 [M+H]⁺.

### Example 1.284

### Preparation of N-(4-(4-Chloro-2-(4-(3,3,3-trifluoropropyl)piperazin-1-yl)phenylcarbamoyl)-2,3-difluorobenzyl)-2-hydroxynicotinamide (Compound 288)

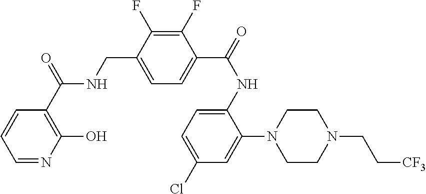 [SMILES: O=C(NCc1ccc(C(=O)Nc2ccc(Cl)cc2N2CCN(CCC(F)(F)F)CC2)c(F)c1F)c1cccnc1O]

From Intermediate 3 and 2-hydroxynicotinic acid, the title compound was obtained using a method similar to the one described in Example 1.274. LCMS m/z=598.4 [M+H]⁺.

### Example 1.289

### Preparation of N-(4-(4-Chloro-2-(4-(3,3,3-trifluoropropyl)piperazin-1-yl)phenylcarbamoyl)-2,3-difluorobenzyl)-5-hydroxypyrazine-2-carboxamide (Compound 294)

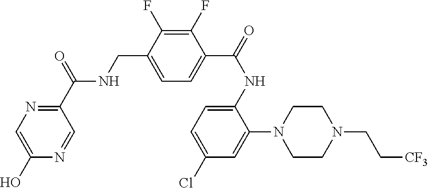 [SMILES: O=C(NCc1ccc(C(=O)Nc2ccc(Cl)cc2N2CCN(CCC(F)(F)F)CC2)c(F)c1F)c1cnc(O)cn1]

From Intermediate 3 and 5-hydroxypyrazine-2-carboxylic acid, the title compound was obtained using a method similar to the one described in Example 1.274. LCMS m/z=599.4 [M+H]⁺. ¹H NMR (400 MHz, DMSO-d₆) δ ppm 3.15-3.72 (m, 12H), 4.52 (d, J=6.1 Hz, 2H), 7.25 (m, 4H), 7.62 (m, 1H), 7.98 (m, 2H), 8.13 (m, 1H), 9.11 (m, 1H), 9.75 (d, J=4.4 Hz, 1H).

### Example 1.295

### Preparation of N-(4-Chloro-2-(4-(3,3,3-trifluoropropyl)piperazin-1-yl)phenyl)-2,3-difluoro-4-((1-hydroxycyclopropanecarboxamido)methyl)benzamide (Compound 311)

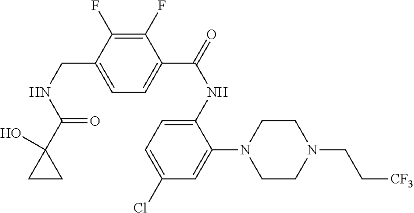 [SMILES: O=C(Nc1ccc(Cl)cc1N1CCN(CCC(F)(F)F)CC1)c1ccc(CNC(=O)C2(O)CC2)c(F)c1F]

From Intermediate 3 and 1-hydroxycyclopropanecarboxylic acid, the title compound was obtained using a method similar to the one described in Example 1.274. LCMS m/z=561.4 [M+H]⁺.

### Example 1.296

### Preparation of N-(4-Chloro-2-(4-(3,3,3-trifluoropropyl)piperazin-1-yl)phenyl)-2,3-difluoro-4-(((1r,4r)-4-hydroxycyclohexanecarboxamido)methyl)benzamide (Compound 312)

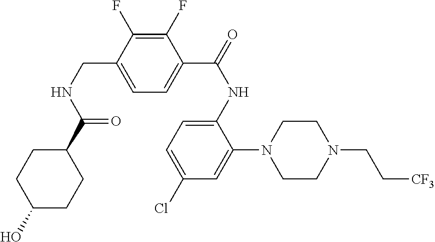 [SMILES: O=C(Nc1ccc(Cl)cc1N1CCN(CCC(F)(F)F)CC1)c1ccc(CNC(=O)[C@H]2CC[C@H](O)CC2)c(F)c1F]

From Intermediate 3 and trans-4-hydroxycyclohexanecarboxylic acid, the title compound was obtained using a method similar to the one described in Example 1.274. LCMS m/z=603.4 [M+H]⁺. ¹H NMR (400 MHz, DMSO-d₆) δ ppm 1.10˜1.29 (m, 4H), 1.78˜1.80 (m, 4H), 2.20 (m, 1H), 3.12˜3.69 (br, 14H), 4.31 (d, J=5.8 Hz, 2H), 7.14˜7.33 (m, 3H), 7.62 (m, HD, 8.12 (m, HD, 8.41 (m, HD, 9.76 (d, J=4.5 Hz, HD.

### Example 1.297

### Preparation of N-(4-Chloro-2-(4-(3,3,3-trifluoropropyl)piperazin-1-yl)phenyl)-2,3-difluoro-4-(((1r,4r)-4-(hydroxymethyl)cyclohexanecarboxamido)methyl)benzamide (Compound 329)

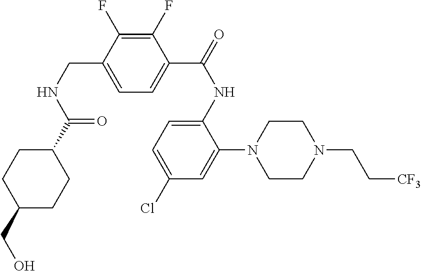 [SMILES: O=C(Nc1ccc(Cl)cc1N1CCN(CCC(F)(F)F)CC1)c1ccc(CNC(=O)[C@H]2CC[C@H](CO)CC2)c(F)c1F]

From Intermediate 3 and (1r,4r)-4-(hydroxymethyl)cyclohexanecarboxylic acid, the title compound was obtained using a method similar to the one described in Example 1.274. LCMS m/z=617.6 [M+H]⁺.

### Example 1.298

### Preparation of N-(4-Chloro-2-(4-(3,3,3-trifluoropropyl)piperazin-1-yl)phenyl)-2,3-difluoro-4-(((1s,4s)-4-hydroxycyclohexanecarboxamido)methyl)benzamide (Compound 330)

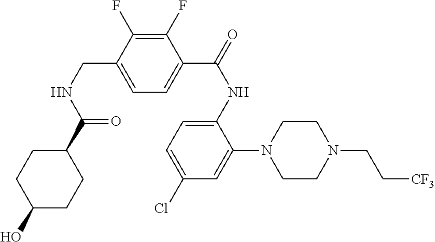 [SMILES: O=C(Nc1ccc(Cl)cc1N1CCN(CCC(F)(F)F)CC1)c1ccc(CNC(=O)[C@H]2CC[C@@H](O)CC2)c(F)c1F]

From Intermediate 3 and (1s,4s)-4-hydroxycyclohexanecarboxylic acid, the title compound was obtained using a method similar to the one described in Example 1.274. LCMS m/z=603.6 [M+H]⁺. ¹H NMR (400 MHz, DMSO-d₆) δ ppm 1.12˜1.32 (m, 4H), 1.72˜1.85 (m, 4H), 2.21 (m, 1H), 3.11˜3.62 (br, 14H), 4.32 (d, J=5.7 Hz, 2H), 7.15˜7.31 (m, 3H), 7.65 (m, 1H), 8.12 (m, 1H), 8.45 (m, 1H), 9.79 (d, J=4.5 Hz, 1H).

### Example 1.300

### Preparation of (R)—N-(4-(4-Chloro-2-(4-(3,3,3-trifluoropropyl)piperazin-1-yl)phenylcarbamoyl)-2,3-difluorobenzyl)-4-(2-hydroxyethyl)morpholine-3-carboxamide (Compound 302)

To a solution of (R)—N-(4-(4-chloro-2-(4-(3,3,3-trifluoropropyl)piperazin-1-yl)phenylcarbamoyl)-2,3-difluorobenzyl)morpholine-3-carboxamide (30 mg, 0.051 mmol) in DMSO (1 mL), was added 2-bromoethanol (6.35 mg, 0.051 mmol) followed by DIEA (6.1 mg, 0.051 mmol). After stirring at 50° C. for 1 h, the reaction was poured into water and extracted with ethyl acetate. The organic layer was dried over MgSO₄ and concentrated under reduced pressure. The residue was purified by HPLC to give the title compound (18 mg, 55.8%). LCMS m/z=635.10 [M+H]⁺.

### Example 1.301

### Preparation of (R)—N-(4-(4-Chloro-2-(4-(3,3,3-trifluoropropyl)piperazin-1-yl)phenylcarbamoyl)-2,3-difluorobenzyl)-4-ethylmorpholine-3-carboxamide (Compound 303)

From (R)—N-(4-(4-chloro-2-(4-(3,3,3-trifluoropropyl)piperazin-1-yl)phenylcarbamoyl)-2,3-difluorobenzyl)morpholine-3-carboxamide and bromoethane, the title compound was obtained using a method similar to the one described in Example 1.300. LCMS m/z=619.10 [M+H]⁺.

### Example 1.302

### Preparation of (R)—N-(4-(4-Chloro-2-(4-(3,3,3-trifluoropropyl)piperazin-1-yl)phenylcarbamoyl)-2,3-difluorobenzyl)-4-(2-hydroxyethyl)thiomorpholine-3-carboxamide (Compound 304)

### Step A: Preparation of (R)—N-(4-(4-Chloro-2-(4-(3,3,3-trifluoropropyl)piperazin-1-yl)phenylcarbamoyl)-2,3-difluorobenzyl)thiomorpholine-3-carboxamide (Compound 273)

To a solution of Intermediate 3 (0.2 g, 0.419 mmol) in DMF (5 mL), were added (R)-4-(tert-butoxycarbonyl)thiomorpholine-3-carboxylic acid (0.104 g, 0.419 mmol) and 2-(3H-[1,2,3]triazolo[4,5-b]pyridin-3-yl)-1,1,3,3-tetramethylisouronium hexafluorophosphate(V) (0.191 g, 0.503 mmol) at an ambient temperature. After stirring for 12 h, the reaction was extracted with ethyl acetate. The organic extract was dried over MgSO₄ and concentrated under reduced pressure to give Compound 454 and was treated with 4.0 M HCl in dioxane (5 mL). After 5 h, the reaction was concentrated under reduced pressure, neutralized with 2.0 M NaOH and then extracted with ethyl acetate. The organic layer was dried over MgSO₄ and concentrated under reduced pressure to give the crude title compound, which was used for the next step without further purification (0.169 g, 66.9%). LCMS m/z=606.50.10 [M+H]⁺.

### Step B: Preparation of (R)—N-(4-(4-Chloro-2-(4-(3,3,3-trifluoropropyl)piperazin-1-yl)phenylcarbamoyl)-2,3-difluorobenzyl)-4-(2-hydroxyethyl)thiomorpholine-3-carboxamide (Compound 304)

From (R)—N-(4-(4-chloro-2-(4-(3,3,3-trifluoropropyl)piperazin-1-yl)phenylcarbamoyl)-2,3-difluorobenzyl)thiomorpholine-3-carboxamide and 2-bromoethanol, the title compound was obtained using a method similar to the one described in Example 1.300. LCMS m/z=650.6 [M+H]⁺.

### Example 1.303

### Preparation of (R)—N-(4-(4-Chloro-2-(4-(3,3,3-trifluoropropyl)piperazin-1-yl)phenylcarbamoyl)-2,3-difluorobenzyl)-4-ethylthiomorpholine-3-carboxamide (Compound 305)

From (R)—N-(4-(4-chloro-2-(4-(3,3,3-trifluoropropyl)piperazin-1-yl)phenylcarbamoyl)-2,3-difluorobenzyl)thiomorpholine-3-carboxamide and bromoethane, the title compound was obtained using a method similar to the one described in Example 1.300. LCMS m/z=634.6 [M+H]⁺.

### Example 1.304

### Preparation of (1r,4r)-4-(4-(4-Chloro-2-(4-(3,3,3-trifluoropropyl)piperazin-1-yl)phenylcarbamoyl)-2,3-difluorobenzylcarbamoyl)cyclohexyl dihydrogen phosphate (Compound 381)

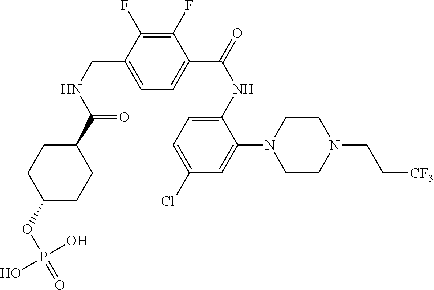 [SMILES: O=C(Nc1ccc(Cl)cc1N1CCN(CCC(F)(F)F)CC1)c1ccc(CNC(=O)[C@H]2CC[C@H](OP(=O)(O)O)CC2)c(F)c1F]

To a solution of N-(4-chloro-2-(4-(3,3,3-trifluoropropyl)piperazin-1-yl)phenyl)-2,3-difluoro-4-(((1r,4r)-4-hydroxycyclohexanecarboxamido)methyl)benzamide (500 mg, 0.829 mmol, Compound 312, see Example 1.296) in THF (2 mL), was added di-tert-butyl diisopropylphosphoramidite (920 mg, 3.32 mmol) followed by 1H-tetrazole (290 mg, 4.15 mmol) at room temperature. After stirring for 2 h, 1-hydroperoxybutane (747 mg, 8.29 mmol) was added to the reaction and stirred for 1 h. The reaction was extracted with ethyl acetate, dried over MgSO₄, and concentrated under reduced pressure. The resulting residue was purified by column chromatography. The t-butyl phosphate compound was treated with 50% TFA in DCM (5 mL). After 5 h, the reaction was concentrated under reduced pressure to provide the TFA salt of Compound 381. The resulting salt was dissolved in acetonitrile (1 mL) and 2.0M aqueous NaOH (3 mL) and then purified by C-18 reverse phase column chromatography (10% acetonitrile in H₂O) to give the title compound (205 mg, 34.0%). LCMS m/z=683.0 [M+H]⁺. ¹H NMR (400 MHz, DMSO-d₆) δ ppm 1.13-1.52 (m, 4H), 1.73-1.95 (m, 4H), 2.23 (m, 1H), 3.11˜3.62 (br, 14H), 4.34 (d, J=5.7 Hz, 2H), 7.18˜7.31 (m, 2H), 7.32 (m, 1H), 8.23 (m, 1H), 8.55 (m, 1H), 9.82 (d, T=8.1 Hz, 1H).

### Example 1.305

### Preparation of (1s,4s)-4-(4-(4-Chloro-2-(4-(3,3,3-trifluoropropyl)piperazin-1-yl)phenylcarbamoyl)-2,3-difluorobenzylcarbamoyl)cyclohexyl dihydrogen phosphate (Compound 392)

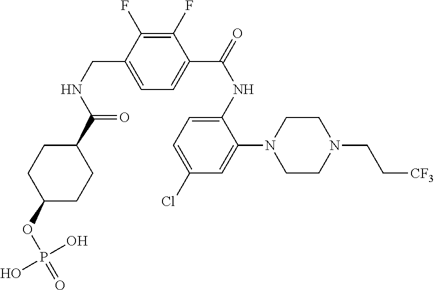 [SMILES: O=C(Nc1ccc(Cl)cc1N1CCN(CCC(F)(F)F)CC1)c1ccc(CNC(=O)[C@H]2CC[C@@H](OP(=O)(O)O)CC2)c(F)c1F]

From N-(4-chloro-2-(4-(3,3,3-trifluoropropyl)piperazin-1-yl)phenyl)-2,3-difluoro-4-(((1s,4s)-4-hydroxycyclohexanecarboxamido)methyl)benzamide hydrochloride and di-tert-butyl diisopropylphosphoramidite, the title compound was obtained using a method similar to the one described in Example 1.304. LCMS m/z=683.0 [M+H]⁺. LCMS m/z=683.0 [M+H]⁺. ¹H NMR (400 MHz, DMSO-d₆) δ ppm 1.13-1.52 (m, 4H), 2.22-1.93 (m, 5H), 3.12-3.60 (br, 14H), 4.31 (d, J=5.7 Hz, 2H), 7.19-7.30 (m, 2H), 7.32 (m, 1H), 8.23 (m, 1H), 8.51 (m, 1H), 9.81 (d, J=8.0 Hz, 1H).

### Example 1.306

### Preparation of N-(4-Chloro-2-(4-(3,3,3-trifluoropropyl)piperazin-1-yl)phenyl)-1,2,3,4-tetrahydroisoquinoline-7-carboxamide (Compound 442), and 2-Butyryl-N-(4-Chloro-2-(4-(3,3,3-trifluoropropyl)piperazin-1-yl)phenyl)-1,2,3,4-tetrahydroisoquinoline-7-carboxamide (Compound 256)

### Step A: Preparation of 2-tert-Butyl 7-methyl 3,4-dihydroisoquinoline-2,7(1H)-dicarboxylate

To a mixture of methyl 1,2,3,4-tetrahydroisoquinoline-7-carboxylate hydrochloride (395 mg, 1.735 mmol) in CH₂Cl₂ (3 mL) were added DIEA (909 μL, 5.20 mmol) and Boc anhydride (443 μL, 1.908 mmol). The reaction was stirred at room temperature for an hour. After this time the solvent was evaporated and the residue was purified by silica gel column chromatography to give the title compound (450 mg, 85%). LCMS m/z=292.2 [M+H]⁺; ¹H NMR (400 MHz, DMSO-d₆) δ ppm 1.43 (s, 9H) 2.84 (t, J=5.87 Hz, 2H) 3.56 (t, J=5.87 Hz, 2H) 3.84 (s, 3H) 4.56 (s, 2H) 7.31 (d, J=7.83 Hz, 1H) 7.73-7.76 (m, 1H) 7.77 (s, 1H).

### Step B: Preparation of 2-(tert-Butoxycarbonyl)-1,2,3,4-tetrahydroisoquinoline-7-carboxylic acid

2-tert-Butyl 7-methyl 3,4-dihydroisoquinoline-2,7(1H)-dicarboxylate (450 mg, 1.545 mmol) was dissolved in THF (2 mL) and MeOH (1 mL). LiOH (111 mg, 4.63 mmol) and H₂O (1 mL) were added. The reaction was heated at 60° C. for 3 hours. The excess solvent was evaporated, until only largely H₂O remained. This aqueous layer was cooled on an ice bath and then made slightly acidic by the dropwise addition of 5M HCl. The reaction was added H₂O (10 mL) and extracted with EtOAc (10 mL). The aqueous layer was extracted again with EtOAc (10 mL). The organic layers were combined, dried, and concentrated to give a colorless oil that solidified under vacuum overnight. This solid precipitate was suspended in MTBE, filtered, and dried to give the title compound (260 mg, 58.3%). LCMS m/z=278.2 [M+H]⁺; ¹H NMR (400 MHz, DMSO-d₆) δ ppm (s, 9H) 2.83 (t, J=5.87 Hz, 2H) 3.56 (t, J=5.94 Hz, 2H) 4.55 (s, 2H) 7.28 (d, J=7.96 Hz, 1H) 7.70-7.76 (m, 2H) 12.84 (bs, 1H).

### Step C: Preparation of tert-Butyl 7-(4-Chloro-2-(4-(3,3,3-trifluoropropyl)piperazin-1-yl)phenylcarbamoyl)-3,4-dihydroisoquinoline-2(1H)-carboxylate

2-(tert-Butoxycarbonyl)-1,2,3,4-tetrahydroisoquinoline-7-carboxylic acid (260 mg, 0.938 mmol), 4-chloro-2-(4-(3,3,3-trifluoropropyl)piperazin-1-yl)aniline (289 mg, 0.938 mmol), DIEA (246 μL, 1.406 mmol), and HATU (428 mg, 1.125 mmol) were added to CH₂Cl₂ (6 mL) and DMF (1 mL). The reaction was stirred at reflux for 4 hours. An extraction was performed (10 mL each of H₂O and CH₂Cl₂). The aqueous layer was extracted again with CH₂Cl₂ (10 mL). The organic layers were combined, dried, concentrated, and the residue was purified by silica gel column chromatography to give the title compound (270 mg, 49.8%). LCMS m/z=567.6 [M+H]⁺; ¹H NMR (400 MHz, DMSO-d₆) δ ppm 1.43 (s, 9H) 2.54-2.64 (m, 7H) 2.83-2.93 (m, 7H) 3.59 (t, J=5.87 Hz, 2H) 4.60 (s, 2H) 7.20 (dd, J=8.65, 2.34 Hz, 1H) 7.29 (d, J=2.40 Hz, 1H) 7.37 (d, J=8.34 Hz, 1H) 7.72-7.77 (m, 2H) 8.07 (d, J=8.59 Hz, 1H) 9.46 (s, 1H).

### Step D: Preparation of N-(4-Chloro-2-(4-(3,3,3-trifluoropropyl)piperazin-1-yl)phenyl)-1,2,3,4-tetrahydroisoquinoline-7-carboxamide (Compound 442)

tert-Butyl 7-(4-chloro-2-(4-(3,3,3-trifluoropropyl)piperazin-1-yl)phenylcarbamoyl)-3,4-dihydroisoquinoline-2(1H)-carboxylate (270 mg, 0.476 mmol) was dissolved in CH₂Cl₂ (3 mL). HCl (4M in dioxane) (2381 μL, 9.52 mmol) was added to the solution and stirred at room temperature overnight (a gummy precipitate formed). The next day, the reaction was extracted under basic conditions (50 mL each of 3M NaOH/H₂O and CH₂Cl₂). The aqueous layer was extracted again with CH₂Cl₂ (50 mL). The organic layers were combined, dried, and concentrated to give the title compound (190.2 mg, 82%). LCMS m/z=467.2 [M+H]⁺; ¹H NMR (400 MHz, DMSO-d₆) δ ppm 2.54-2.65 (m, 7H) 2.82 (t, J=5.75 Hz, 2H) 2.85-2.94 (m, 5H) 3.06 (t, J=5.94 Hz, 2H) 4.01 (s, 2H) 7.16-7.24 (m, 1H) 7.26-7.35 (m, 2H) 7.64 (s, 1H) 7.70 (dd, J=7.89, 1.58 Hz, 1H) 8.08 (d, J=8.72 Hz, 1H) 9.42 (s, 1H).

### Step E: Preparation of 2-Butyryl-N-(4-chloro-2-(4-(3,3,3-trifluoropropyl)piperazin-1-yl)phenyl)-1,2,3,4-tetrahydroisoquinoline-7-carboxamide (Compound 256)

N-(4-Chloro-2-(4-(3,3,3-trifluoropropyl)piperazin-1-yl)phenyl)-1,2,3,4-tetrahydroisoquinoline-7-carboxamide (11 mg, 0.024 mmol) and DIEA (12.34 μL, 0.071 mmol) were added to a vial with DMF (0.1 mL). Butyryl chloride (1.2 eq) was then added. The reaction was stirred at room temperature for a half hour. The mixture was purified by preparative LC/MS to give the title compound (6.3 mg, 40.7%). LCMS m/z=537.4 [M+H]⁺; ¹H NMR (400 MHz, DMSO-d₆) δ ppm 0.86-0.95 (m, 3H) 1.49-1.60 (m, 2H) 2.39 (t, J=7.26 Hz, 2H) 2.69-2.82 (m, 4H) 2.82-2.87 (m, 2H) 2.94 (t, J=5.37 Hz, 2H) 3.00-3.19 (m, 6H) 3.71 (t, J=5.94 Hz, 2H) 4.71 (s, 1H) 4.75 (s, 1H) 7.20-7.28 (m, J=8.59 Hz, 1H) 7.31 (d, J=2.27 Hz, 1H) 7.36 (d, J=7.96 Hz, 1H) 7.71-7.86 (m, 2H) 7.99 (dd, J=24.06, 8.65 Hz, 1H) 9.44 (s, 1H).

### Examples 1.307 to 1.310

The following compounds were prepared using the disclosed intermediates and a method similar to the one described in Example 1.306, Step E.

| | | | Product |
|---|---|---|---|
| Example | Cmpd | | (Mass Observed |
| No. | No. | Intermediates Used | LC/MS m/z) |
| 1.307 | 257 | N-(4-chloro-2-(4-(3,3,3- | 565.2 [M + H]⁺ |
| | | trifluoropropyl)piperazin- | |
| | | 1-yl)phenyl)-1,2,3,4- | |
| | | tetrahydroisoquinoline- | |
| | | 7-carboxamide and 2- | |
| | | ethylbutanoyl chloride | |
| 1.308 | 258 | N-(4-chloro-2-(4-(3,3,3- | 577.2 [M + H]⁺ |
| | | trifluoropropyl)piperazin- | |
| | | 1-yl)phenyl)-1,2,3,4- | |
| | | tetrahydroisoquinoline- | |
| | | 7-carboxamide and thiophene- | |
| | | 2-carbonyl chloride | |
| 1.309 | 259 | N-(4-chloro-2-(4-(3,3,3- | 572.0 [M + H]⁺ |
| | | trifluoropropyl)piperazin- | |
| | | 1-yl)phenyl)-1,2,3,4- | |
| | | tetrahydroisoquinoline- | |
| | | 7-carboxamide and | |
| | | nicotinoyl chloride | |
| 1.310 | 260 ¹ | N-(4-chloro-2-(4-(3,3,3- | 577.6 [M + H]⁺ |
| | | trifluoropropyl)piperazin- | |
| | | 1-yl)phenyl)-1,2,3,4- | |
| | | tetrahydroisoquinoline- | |
| | | 7-carboxamide and 2- | |
| | | cyclopentylacetyl chloride | |

¹ Compound 260): ¹H NMR (400 MHz, DMSO-d₆) δ ppm 1.08-1.21 (m, 2 H) 1.42-1.67 (m, 4 H) 1.69-1.82 (m, 2 H) 2.12-2.24 (m, 1 H) 2.40-2.46 (m, 2 H) 2.76-2.88 (m, 3 H) 2.93 (t, J = 5.62 Hz, 2 H) 3.07-3.22 (m, 5 H) 3.26-3.40 (m, 4 H) 3.67-3.75 (m, 2 H) 4.71 (s, 1 H) 4.76 (s, 1 H) 7.22-7.28 (m, 1 H) 7.30-7.33 (m, 1 H) 7.36 (d, J = 7.96 Hz, 1 H) 7.72-7.84 (m, 2 H) 7.91-8.04 (m, 1 H) 9.43 (d, J = 4.04 Hz, 1 H).

### Example 1.311

### Preparation of N-(4-Chloro-2-(4-(3,3,3-trifluoropropyl)piperazin-1-yl)phenyl)-2,3-difluoro-4-((2-oxopyrrolidin-1-yl)methyl)benzamide (Compound 286)

Intermediate 1 (10 mg, 0.018 mmol, see Example 1.20), methyl 4-aminobutanoate (2.166 mg, 0.018 mmol), and DIEA (3.23 μL, 0.018 mmol) were added to a vial with DMF (0.2 μL). The reaction was stirred at 80° C. for two hours. LiOH (1.329 mg, 0.055 mmol) and H₂O (0.1 mL) were added. The reaction was heated at 80° C. until reaction was complete. The mixture was purified by preparative LC/MS to give the title compound (3.5 mg, 28.7%). LCMS m/z=545.4 [M+H]⁺; ¹H NMR (400 MHz, DMSO-d₆) δ ppm 1.89-2.02 (m, 2H) 2.25-2.35 (m, 2H) 2.77-2.98 (m, 3H) 2.99-3.31 (m, 11H) 4.53 (s, 2H) 7.19-7.32 (m, 2H) 7.36 (d, J=2.27 Hz, 1H) 7.63 (t, J=7.14 Hz, 1H) 8.16 (d, J=8.46 Hz, 1H) 9.71 (d, J=4.67 Hz, 1H).

### Example 1.312

### Preparation of (S)—N-(4-(4-Chloro-2-(4-(3,3,3-trifluoropropyl)piperazin-1-yl)phenylcarbamoyl)-2,3-difluorobenzyl)-4-methylmorpholine-2-carboxamide (Compound 296)

N-(4-(4-chloro-2-(4-(3,3,3-trifluoropropyl)piperazin-1-yl)phenylcarbamoyl)-2,3-difluorobenzyl)morpholine-2-carboxamide dihydrochloride (10 mg, 0.015 mmol, di HCl salt of Compound 264, see Example 1.259), iodomethane (1.13 μL, 0.018 mmol), and DIEA (50.0 μL, 0.286 mmol) were dissolved in DMF (0.2 mL). The reaction was stirred at room temperature overnight. The reaction was then heated to 80° C. until completion. The mixture was purified by preparative HPLC to give the title compound (1.9 mg, 14.99%). LCMS m/z=604.2 [M+H]⁺; ¹H NMR (400 MHz, DMSO-d₆) δ ppm 2.76-2.94 (m, 5H) 2.95-3.29 (m, 10H) 3.63-3.92 (m, 4H) 4.11 (d, J=6.57 Hz, 1H) 4.13-4.21 (m, 1H) 4.25-4.34 (m, 1H) 4.39-4.49 (m, 2H) 4.71 (dd, J=11.62, 2.40 Hz, 1H) 7.19-7.32 (m, 2H) 7.37 (d, J=2.02 Hz, 1H) 7.62 (t, J=7.33 Hz, 1H) 8.17 (d, J=8.34 Hz, 1H) 8.76-8.88 (m, 1H) 9.70 (d, J=5.05 Hz, 1H).

### Examples 1.313 to 1.315

The following compounds were prepared using the disclosed intermediates and a method similar to the one described in Example 1.312.

| | | | Product |
|---|---|---|---|
| Example | Cmpd | | (Mass Observed |
| No. | No. | Intermediates Used | LC/MS m/z) |
| 1.313 | 297 ¹ | N-(4-(4-chloro-2-(4- | 618.4 [M + H]⁺ |
| | | (3,3,3-trifluoro- | |
| | | propyl)piperazin-1- | |
| | | yl)phenylcarbamoyl)- | |
| | | 2,3-difluoro- | |
| | | benzyl)morpholine-2- | |
| | | carboxamide dihydro- | |
| | | chloride and ethyl | |
| | | iodide | |
| 1.314 | 298 | N-(4-(4-chloro-2-(4- | 634.8 [M + H]⁺ |
| | | (3,3,3-trifluoro- | |
| | | propyl)piperazin-1- | |
| | | yl)phenylcarbamoyl)- | |
| | | 2,3-difluoro- | |
| | | benzyl)morpholine-2- | |
| | | carboxamide dihydro- | |
| | | chloride and 2-bromo- | |
| | | ethanol | |
| 1.315 | 299 ² | N-(4-(4-chloro-2-(4- | 674.6 [M + H]⁺ |
| | | (3,3,3-trifluoro- | |
| | | propyl)piperazin-1- | |
| | | yl)phenylcarbamoyl)- | |
| | | 2,3-difluoro- | |
| | | benzyl)morpholine-2- | |
| | | carboxamide dihydro- | |
| | | chloride and 1-bromo- | |
| | | 3,3-dimethylbutane | |

¹ Compound 297: ¹H NMR (400 MHz, DMSO-d₆) δ ppm 1.20-1.27 (m, 3 H) 2.74-2.90 (m, 3 H) 2.91-3.25 (m, 15 H) 3.68 (d, J = 12.00 Hz, 1 H) 3.72-3.85 (m, 1 H) 4.19 (d, J = 11.87 Hz, 1 H) 4.27-4.34 (m, 1 H) 4.44 (d, J = 5.18 Hz, 2 H) 7.19-7.32 (m, 2 H) 7.37 (d, J = 2.27 Hz, 1 H) 7.63 (t, J = 6.88 Hz, 1 H) 8.18 (d, J = 8.34 Hz, 1 H) 8.80 (t, J = 5.37 Hz, 1 H) 9.70 (d, J = 5.43 Hz, 1 H).
² Compound 299: ¹H NMR (400 MHz, DMSO-d₆) δ ppm 0.87-0.95 (m, 9 H) 1.53-1.61 (m, 1 H) 2.78-2.96 (m, 3 H) 2.95-3.27 (m, 10 H) 3.28-3.45 (m, 4 H) 3.72-4.07 (m, 4 H) 4.18 (d, J = 11.49 Hz, 1 H) 4.32 (d, J = 12.63 Hz, 1 H) 4.44 (s, 2 H) 7.17-7.33 (m, 2 H) 7.37 (d, J = 2.27 Hz, 1 H) 7.57-7.66 (m, J = 6.88, 6.88 Hz, 1 H) 8.17 (d, J = 8.46 Hz, 1 H) 8.81 (t, J = 5.43 Hz, 1 H) 9.70 (d, J = 4.80 Hz, 1 H).

### Example 1.316

### Preparation of HCl Salt of (R)—N-(4-Chloro-2-(4-(3,3,3-trifluoropropyl)piperazin-1-yl)phenyl)-2,3-difluoro-4-((3-hydroxybutanamido)methyl)benzamide (Compound 313)

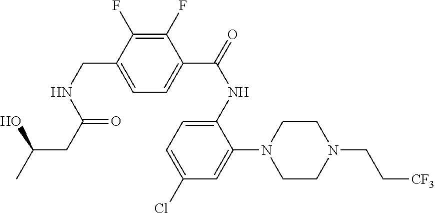 [SMILES: C[C@@H](O)CC(=O)NCc1ccc(C(=O)Nc2ccc(Cl)cc2N2CCN(CCC(F)(F)F)CC2)c(F)c1F]

Intermediate 3 (10 mg, 0.021 mmol), (R)-3-hydroxybutanoic acid (2.40 mg, 0.023 mmol), DIEA (20 μL, 0.115 mmol), and HATU (9.57 mg, 0.025 mmol) were added to a vial with DMF (0.3 mL). The reaction was stirred at 80° C. for an hour. The mixture was purified by preparative HPLC. The resulting material was re-dissolved in ACN (0.4 mL). HCl (4M in dioxane) (21 μL, 0.084 mmol) was added and the reaction was stirred at room temperature for an hour. Then, the excess solvent was removed and the residue was re-dissolved in ACN (0.5 mL) and H₂O (0.5 mL), frozen, and lyophilized to give the title compound. LCMS m/z=563.2 [M+H]⁺.

### Examples 1.317, 1.319 to 1.324, 1.328 to 1.347

The following compounds were prepared using the disclosed intermediates and a method similar to the one described in Example 1.316.

| | | | Product |
|---|---|---|---|
| Example | Cmpd | | (Mass Observed |
| No. | No. | Intermediates Used | LC/MS m/z) |
| 1.317 | 314 | Intermediate 3 and 3-hydroxy-2,2- | 577.6 [M + H]⁺ |
| | | dimethylpropanoic acid | |
| 1.319 | 316 | Intermediate 3 and 4-hydroxybutanoic acid | 563.4 [M + H]⁺ |
| 1.320 | 317 | Intermediate 3 and 2-ethyl-2-hydroxybutanoic | 591.4 [M + H]⁺ |
| | | acid | |
| 1.321 | 318 | Intermediate 3 and 2- | 603.6 [M + H]⁺ |
| | | hydroxycyclohexanecarboxylic acid | |
| 1.322 | 319 | Intermediate 3 and (R)-2-cyclohexyl-2- | 617.4 [M + H]⁺ |
| | | hydroxyacetic acid | |
| 1.323 | 320 | Intermediate 3 and 3-hydroxy-3- | 577.6 [M + H]⁺ |
| | | methylbutanoic acid | |
| 1.324 | 321 | Intermediate 3 and (S)-2-hydroxy-4- | 591.4 [M + H]⁺ |
| | | methylpentanoic acid | |
| 1.328 | 324 | Intermediate 3 and 4-(tert- | N.D. ¹ |
| | | butoxycarbonyl)thiomorpholine-3-carboxylic | |
| | | acid | |
| | 327 | Deprotection | 606.4 [M + H]⁺ |
| 1.329 | 328 | Intermediate 3 and (R)-2-(1-(tert- | 602.6 [M + H]⁺ |
| | | butoxycarbonyl)piperidin-2-yl)acetic acid | |
| 1.330 | 331 | Intermediate 3 and 3- | 576.6 [M + H]⁺ |
| | | (dimethylamino)propanoic acid | |
| 1.331 | 464 | Intermediate 3 and (S)-2-(1-(tert- | N.D. ¹ |
| | | butoxycarbonyl)pyrrolidin-3-yl)acetic acid | |
| | 332 | Deprotection | 588.4 [M + H]⁺ |
| 1.332 | 333 | Intermediate 3 and 3-(piperidin-1-yl)propanoic | 616.4 [M + H]⁺ |
| | | acid | |
| 1.333 | 465 | Intermediate 3 and (1r,4r)-4-(tert- | N.D. ¹ |
| | | butoxycarbonylamino)cyclohexanecarboxylic | |
| | | acid | |
| | 334 | Deprotection | 602.6 [M + H]⁺ |
| 1.334 | 466 | Intermediate 3 and (S)-2-(1-(tert- | N.D. ¹ |
| | | butoxycarbonyl)piperidin-2-yl)acetic acid | |
| | 335 | Deprotection | 602.0 [M + H]⁺ |
| 1.335 | 467 | Intermediate 3 and (S)-1-(tert- | N.D. ¹ |
| | | butoxycarbonyl)pyrrolidine-3-carboxylic acid | |
| | 336 | Deprotection | 574.4 [M + H]⁺ |
| 1.336 | 337 | Intermediate 3 and 3-(diethylamino)propanoic | 604.6 [M + H]⁺ |
| | | acid | |
| 1.337 | 468 | Intermediate 3 and 2-((1s,4s)-4-(tert- | N.D. ¹ |
| | | butoxycarbonylamino)cyclohexyl)acetic acid | |
| | 338 | Deprotection | 616.4 [M + H]⁺ |
| 1.338 | 339 | Intermediate 3 and 3-morpholinopropanoic acid | 618.4 [M + H]⁺ |
| | | hydrochloride | |
| 1.339 | 340 | Intermediate 3 and 1-methylpiperidine-4- | 602.6 [M + H]⁺ |
| | | carboxylic acid hydrochloride | |
| 1.340 | 469 | Intermediate 3 and (1s,4s)-4-(tert- | N.D. ¹ |
| | | butoxycarbonylamino)cyclohexanecarboxylic | |
| | | acid | |
| | 341 | Deprotection | 602.4 [M + H]⁺ |
| 1.341 | 470 | Intermediate 3 and (1R,3S)-3-(tert- | N.D. ¹ |
| | | butoxycarbonylamino)cyclohexanecarboxylic | |
| | | acid | |
| | 342 | Deprotection | 602.6 [M + H]⁺ |
| 1.342 | 471 | Intermediate 3 and (S)-2-(tert- | N.D. ¹ |
| | 472 | butoxycarbonylamino)pentanedioic acid | N.D. ¹ |
| | 343 | Deprotection ² | 606.6 [M + H]⁺ |
| | 344 | | |
| 1.343 | 473 | Intermediate 3 and (1R,3S)-3-(tert- | N.D. ¹ |
| | | butoxycarbonylamino)cyclopentanecarboxylic | |
| | | acid | |
| | 345 | Deprotection | 588.4 [M + H]⁺ |
| 1.344 | 346 | Intermediate 3 and 1-methylpiperidine-3- | 602.6 [M + H]⁺ |
| | | carboxylic acid | |
| 1.345 | 474 | Intermediate 3 and (R)-2-(1-(tert- | N.D. ¹ |
| | | butoxycarbonyl)piperidin-3-yl)acetic acid | |
| | 347 | Deprotection | 602.4 [M + H]⁺ |
| 1.346 | 475 | Intermediate 3 and (R)-2-(1-(tert- | N.D. ¹ |
| | | butoxycarbonyl)pyrrolidin-3-yl)acetic acid | |
| | 348 | Deprotection | 588.4 [M + H]⁺ |
| 1.347 | 476 | Intermediate 3 and 1-(tert- | N.D. ¹ |
| | | butoxycarbonyl)azetidine-3-carboxylic acid | |
| | 349 | Deprotection | 560.4 [M + H]⁺ |

¹ Not Determined
² Mixture of Compounds 343 and 344.

### Example 1.318

### Preparation of (S)—N-(4-Chloro-2-(4-(3,3,3-trifluoropropyl)piperazin-1-yl)phenyl)-2,3-difluoro-4-((3-hydroxybutanamido)methyl)benzamide (Compound 315)

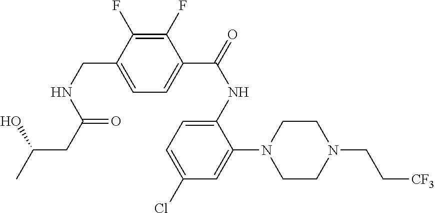 [SMILES: C[C@H](O)CC(=O)NCc1ccc(C(=O)Nc2ccc(Cl)cc2N2CCN(CCC(F)(F)F)CC2)c(F)c1F]

From Intermediate 3 and (S)-3-hydroxybutanoic acid, using a method similar to the one described in Example 1.316, the title compound was obtained. LCMS m/z=563.2 [M+H]⁺.

### Example 1.325

### Preparation of HCl Salt of N-(4-Chloro-2-(4-(3,3,3-trifluoropropyl)piperazin-1-yl)phenyl)-2,3-difluoro-4-((2-hydroxy-2-methylpropanamido)methyl)benzamide (Compound 310)

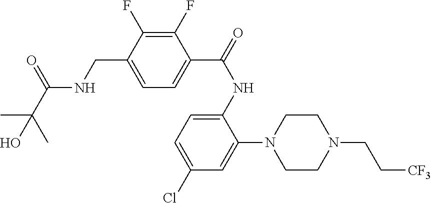 [SMILES: CC(C)(O)C(=O)NCc1ccc(C(=O)Nc2ccc(Cl)cc2N2CCN(CCC(F)(F)F)CC2)c(F)c1F]

From Intermediate 3 and 2-hydroxy-2-methylpropanoic acid, using a method similar to the one described in Example 1.316, the title compound was obtained. LCMS m/z=563.4 [M+H]⁺.

### Example 1.326

### Preparation of HCl Salt of (R)—N-(4-Chloro-2-(4-(3,3,3-trifluoropropyl)piperazin-1-yl)phenyl)-2,3-difluoro-4-((2-hydroxy-4-methylpentanamido)methyl)benzamide (Compound 322)

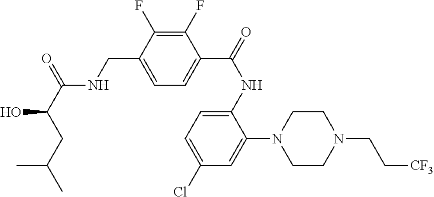 [SMILES: CC(C)C[C@@H](O)C(=O)NCc1ccc(C(=O)Nc2ccc(Cl)cc2N2CCN(CCC(F)(F)F)CC2)c(F)c1F]

From Intermediate 3 and (R)-2-hydroxy-4-methylpentanoic acid, using a method similar to the one described in Example 1.316, the title compound was obtained. LCMS m/z=591.4 [M+H]⁺.

### Example 1.327

### Preparation of HCl Salt of N-(4-Chloro-2-(4-(3,3,3-trifluoropropyl)piperazin-1-yl)phenyl)-2,3-difluoro-4-((3-hydroxy-2-(hydroxymethyl)-2-methylpropanamido)methyl)benzamide (Compound 326)

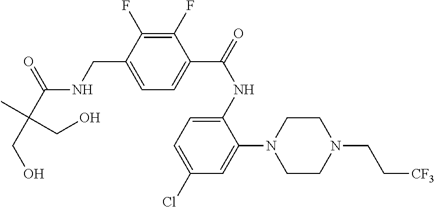 [SMILES: CC(CO)(CO)C(=O)NCc1ccc(C(=O)Nc2ccc(Cl)cc2N2CCN(CCC(F)(F)F)CC2)c(F)c1F]

From Intermediate 3 and 3-hydroxy-2-(hydroxymethyl)-2-methylpropanoic acid, using a method similar to the one described in Example 1.316, the title compound was obtained. LCMS m/z=593.4 [M+H]⁺.

### Example 1.348

### Preparation of (2S,4R)-tert-Butyl 2-(4-(4-chloro-2-(4-(3,3,3-trifluoropropyl)piperazin-1-yl)phenylcarbamoyl)-2,3-difluorobenzylcarbamoyl)-4-hydroxypyrrolidine-1-carboxylate (Compound 323)

Intermediate 3 (10 mg, 0.021 mmol), (2S,4R)-1-(tert-butoxycarbonyl)-4-hydroxypyrrolidine-2-carboxylic acid (5.33 mg, 0.023 mmol), DIEA (20 μL, 0.115 mmol), and HATU (9.57 mg, 0.025 mmol) were added to a vial with DMF (0.3 mL). The reaction was stirred at 80° C. for an hour. The mixture was purified by preparative HPLC to give the title compound (16.1 mg, 92%). LCMS m/z=690.6 [M+H]⁺.

### Examples 1.349, 1.350, 1.352, 1.353, 1.355, and 1.356

The following compounds were prepared using the disclosed intermediates and a method similar to the one described in Example 1.348.

| | | | Product |
|---|---|---|---|
| Example | Cmpd | | (Mass Observed |
| No. | No. | Intermediates Used | LC/MS m/z) |
| 1.349 | 324 | Intermediate 3 and | 706.4 [M + H]⁺ |
| | | 4-(tert-butoxy- | |
| | | carbonyl)thio- | |
| | | morpholine-3- | |
| | | carboxylic acid | |
| 1.350 | 325 | Intermediate 3 and | 702.6 [M + H]⁺ |
| | | (R)-2-(1-(tert-butoxy- | |
| | | carbonyl)piperidin- | |
| | | 2-yl)acetic acid | |
| 1.352 | 479 | Intermediate 3 and | N.D. ¹ |
| | | (S)-2-(tert-butoxy- | |
| | | carbonylamino)-4- | |
| | | methylpentanoic acid | |
| | 386 | Deprotection | 590.4 [M + H]⁺ |
| 1.353 | 480 | Intermediate 3 and | N.D. ¹ |
| | | (S)-2-(tert-butoxy- | |
| | | carbonylamino)-3- | |
| | | cyanopropanoic acid | |
| | 387 | Deprotection | 573.6 [M + H]⁺ |
| 1.355 | 463 | Intermediate 3 and | N.D. ¹ |
| | | (S)-4-amino-2-(tert- | |
| | | butoxycarbonylamino)- | |
| | | 4-oxobutanoic acid | |
| | 389 | Deprotection | 591.4 [M + H]⁺ |
| 1.356 | 449 | Intermediate 3 and | N.D. ¹ |
| | | 3-(tert-butoxy- | |
| | | carbonylamino)-2- | |
| | | hydroxypropanoic acid | |
| | 390 | Deprotection | 564.2 [M + H]⁺ |

¹ Not Determined

### Example 1.351

### Preparation of N-(4-Chloro-2-(4-(3,3,3-trifluoropropyl)piperazin-1-yl)phenyl)-2,3-difluoro-4-((4-(hydroxymethyl)cyclohexanecarboxamido)methyl)benzamide (Compound 385)

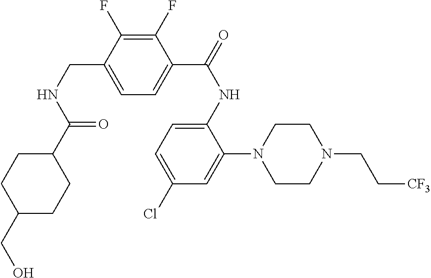 [SMILES: O=C(Nc1ccc(Cl)cc1N1CCN(CCC(F)(F)F)CC1)c1ccc(CNC(=O)C2CCC(CO)CC2)c(F)c1F]

From Intermediate 3 and 4-(hydroxymethyl)cyclohexanecarboxylic acid, using a method similar to the one described in Example 1.348, the title compound was obtained. LCMS m/z=617.4 [M+H]⁺.

### Example 1.354

### Preparation of tert-Butyl 4-(4-(4-Chloro-2-(4-(3,3,3-trifluoropropyl)piperazin-1-yl)phenylcarbamoyl)-2,3-difluorobenzylcarbamoyl)-1,1-dioxotetrahydro-2H-thiopyran-4-ylcarbamate (Compound 481) and 4-Amino-N-(4-(4-chloro-2-(4-(3,3,3-trifluoropropyl)piperazin-1-yl)phenylcarbamoyl)-2,3-difluorobenzyl)-1,1-dioxotetrahydro-2H-thiopyran-4-carboxamide (Compound 388)

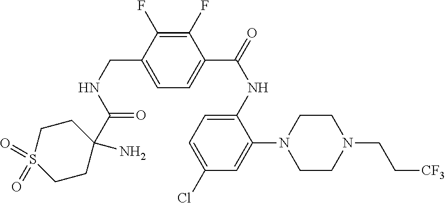 [SMILES: NC1(C(=O)NCc2ccc(C(=O)Nc3ccc(Cl)cc3N3CCN(CCC(F)(F)F)CC3)c(F)c2F)CCS(=O)(=O)CC1]

From Intermediate 3 and 1,1-dioxo-4-(tert-butoxycarbonylamino)tetrahydro-2H-thiopyran-4-carboxylic acid, using a method similar to the one described in Example 1.348, the title compounds were obtained. Compound 449 was deprotected to give Compound 390. Compound 388: LCMS m/z=652.4 [M+H]⁺.

### Example 1.357

### Preparation of N-(4-Chloro-2-(4-(3,3,3-trifluoropropyl)piperazin-1-yl)phenyl)-2,3-difluoro-4-((2-hydroxypropanamido)methyl)benzamide (Compound 391)

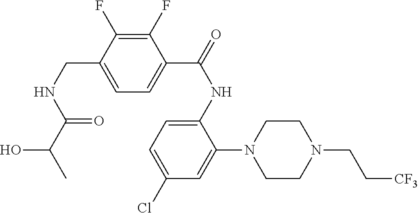 [SMILES: CC(O)C(=O)NCc1ccc(C(=O)Nc2ccc(Cl)cc2N2CCN(CCC(F)(F)F)CC2)c(F)c1F]

From Intermediate 3 and 2-hydroxypropanoic acid, using a method similar to the one described in Example 1.348, the title compound was obtained. LCMS m/z=549.4 [M+H]⁺.

### Example 1.358

### Preparation of N-(4-Chloro-2-(4-(3,3,3-trifluoropropyl)piperazin-1-yl)phenyl)-2,3-difluoro-4-((2-(4-(hydroxymethyl)piperidin-1-yl)acetamido)methyl)benzamide (Compound 350)

Intermediate 3 (10 mg, 0.021 mmol) was dissolved in DMF (0.2 mL). DIEA (7.32 μL, 0.042 mmol) and chloroacetyl chloride (1.680 μL, 0.021 mmol) were added to the solution. The reaction was stirred at room temperature for 15 min. Piperidin-4-ylmethanol (2.90 mg, 0.025 mmol) was added. The reaction was stirred at 80° C. for an hour. The mixture was purified by preparative LC/MS to give the title compound (13.8 mg, 89%). LCMS m/z=632.8 [M+H]⁺; ¹H NMR (400 MHz, DMSO-d₆) δ ppm 1.37-1.54 (m, 2H) 1.54-1.67 (m, 1H) 1.82 (d, T=13.52 Hz, 2H) 2.92-3.11 (m, 4H) 3.13-3.29 (m, 10H) 3.55-3.78 (m, 5H) 3.98 (d, T=4.80 Hz, 2H) 4.50 (d, J=5.56 Hz, 2H) 7.23-7.38 (m, 3H) 7.63 (t, J=7.14 Hz, 1H) 8.16 (d, J=8.46 Hz, 1H) 9.31 (t, J=5.81 Hz, 1H) 9.72 (d, J=4.67 Hz, 1H) 11.54 (bs, 1H).

### Examples 1.359 to 1.387

The following compounds were prepared using the disclosed intermediates and a method similar to the one described in Example 1.358.

| | | | Product |
|---|---|---|---|
| Example | Cmpd | | (Mass Observed |
| No. | No. | Intermediates Used | LC/MS m/z) |
| 1.359 | 351 | Intermediate 3, chloroacetyl chloride, and (S)- | 618.4 [M + H]⁺ |
| | | piperidin-3-ol | |
| 1.360 | 352 | Intermediate 3, chloroacetyl chloride, and | 603.4 [M + H]⁺ |
| | | piperazine | |
| 1.361 | 477 | Intermediate 3, chloroacetyl chloride, and (R)- | N.D. ¹ |
| | | tert-butyl pyrrolidin-3-ylcarbamate | |
| | 353 | Deprotection | 603.4 [M + H]⁺ |
| 1.362 | 355 | Intermediate 3, chloroacetyl chloride, and (R)- | 634.6 [M + H]⁺ |
| | | morpholin-2-ylmethanol | |
| 1.363 | 356 | Intermediate 3, chloroacetyl chloride, and 1- | 645.4 [M + H]⁺ |
| | | propylpiperazine | |
| 1.364 | 357 | Intermediate 3, chloroacetyl chloride, and 1,4- | 631.6 [M + H]⁺ |
| | | diazepan-5-one | |
| 1.365 | 358 | Intermediate 3, chloroacetyl chloride, and | 645.4 [M + H]⁺ |
| | | piperidine-4-carboxamide | |
| 1.366 | 359 | Intermediate 3, chloroacetyl chloride, and (R)- | 631.6 [M + H]⁺ |
| | | pyrrolidine-2-carboxamide | |
| 1.367 | 360 | Intermediate 3, chloroacetyl chloride, and N,N- | 645.0 [M + H]⁺ |
| | | dimethylpiperidin-4-amine | |
| 1.368 | 361 | Intermediate 3, chloroacetyl chloride, and (S)- | 631.6 [M + H]⁺ |
| | | N,N-dimethylpyrrolidin-3-amine | |
| 1.369 | 362 | Intermediate 3, chloroacetyl chloride, and | 618.4 [M + H]⁺ |
| | | piperidin-4-ol | |
| 1.370 | 363 | Intermediate 3, chloroacetyl chloride, and 2,5- | 615.4 [M + H]⁺ |
| | | diazabicyclo[2.2.1]heptane dihydrobromide | |
| 1.371 | 364 | Intermediate 3, chloroacetyl chloride, and (R)- | 631.6 [M + H]⁺ |
| | | N,N-dimethylpyrrolidin-3-amine | |
| 1.372 | 365 | Intermediate 3, chloroacetyl chloride, and (S)- | 643.6 [M + H]⁺ |
| | | octahydropyrrolo[1,2-a]pyrazine | |
| 1.373 | 366 | Intermediate 3, chloroacetyl chloride, and | 632.6 [M + H]⁺ |
| | | piperidin-3-ylmethanol | |
| 1.374 | 478 | Intermediate 3, chloroacetyl chloride, and (S)- | N.D. ¹ |
| | | tert-butyl 2-methylpiperazine-1-carboxylate | |
| | 367 | Deprotection | 617.6 [M + H]⁺ |
| 1.375 | 368 | Intermediate 3, chloroacetyl chloride, and 4- | 616.4 [M + H]⁺ |
| | | methylpiperidine | |
| 1.376 | 369 | Intermediate 3, chloroacetyl chloride, and | 617.0 [M + H]⁺ |
| | | piperazin-2-one | |
| 1.377 | 370 | Intermediate 3, chloroacetyl chloride, and | 646.4 [M + H]⁺ |
| | | piperazine-1-carboxamide | |
| 1.378 | 371 | Intermediate 3, chloroacetyl chloride, and 3- | 616.6 [M + H]⁺ |
| | | methylpiperidine | |
| 1.379 | 372 | Intermediate 3, chloroacetyl chloride, and | 618.4 [M + H]⁺ |
| | | piperidin-3-ol | |
| 1.380 | 373 | Intermediate 3, chloroacetyl chloride, and 1- | 617.4 [M + H]⁺ |
| | | methylpiperazine | |
| 1.3801 | 374 | Intermediate 3, chloroacetyl chloride, and 1- | 631.6 [M + H]⁺ |
| | | ethylpiperazine | |
| 1.3802 | 375 | Intermediate 3, chloroacetyl chloride, and 2- | 646.4 [M + H]⁺ |
| | | (piperidin-2-yl)ethanol | |
| 1.383 | 376 | Intermediate 3, chloroacetyl chloride, and (S)- | 604.6 [M + H]⁺ |
| | | pyrrolidin-3-ol | |
| 1.384 | 377 | Intermediate 3, chloroacetyl chloride, and (R)- | 618.4 [M + H]⁺ |
| | | pyrrolidin-2-ylmethanol | |
| 1.385 | 378 | Intermediate 3, chloroacetyl chloride, and (S)- | 618.2 [M + H]⁺ |
| | | pyrrolidin-2-ylmethanol | |
| 1.386 | 379 | Intermediate 3, chloroacetyl chloride, and | 645.2 [M + H]⁺ |
| | | piperidine-3-carboxamide | |
| 1.387 | 380 | Intermediate 3, chloroacetyl chloride, and (S)- | 631.8 [M + H]⁺ |
| | | pyrrolidine-2-carboxamide | |

¹ Not Determined

### Example 1.388

### Preparation of N-(4-(4-Chloro-2-(4-(3,3,3-trifluoropropyl)piperazin-1-yl)phenylcarbamoyl)-2,3-difluorobenzyl)-5-(hydroxymethyl)-1H-1,2,3-triazole-4-carboxamide (Compound 393)

### Step A: Preparation of N-(4-Chloro-2-(4-(3,3,3-trifluoropropyl)piperazin-1-yl)phenyl)-2,3-difluoro-4-((4-hydroxybut-2-ynamido)methyl)benzamide

A mixture of Intermediate 3 (0.3 g, 0.629 mmol), 4-hydroxybut-2-ynoic acid (0.063 g, 0.629 mmol), HATU (0.359 g, 0.944 mmol) and TEA (0.263 mL, 1.887 mmol) was taken up in DMF (3 mL) and heated to 25° C. for 18 h in a 20 mL sealed scintillation vial. The mixture was purified by preparative HPLC to give the title compound as brown solid. LCMS m/z=559.4 [M+H]⁺.

### Step B: Preparation of N-(4-(4-Chloro-2-(4-(3,3,3-trifluoropropyl)piperazin-1-yl)phenylcarbamoyl)-2,3-difluorobenzyl)-5-(hydroxymethyl)-1H-1,2,3-triazole-4-carboxamide (Compound 393)

N-(4-chloro-2-(4-(3,3,3-trifluoropropyl)piperazin-1-yl)phenyl)-2,3-difluoro-4-((4-hydroxybut-2-ynamido)methyl)benzamide (30 mg, 0.054 mmol) and sodium azide (3.84 mg, 0.059 mmol) were taken up in DMSO (1 mL) and heated to 100° C. for 2 h in a 10 mL heavy walled sealed tube under microwave irradiation. The mixture was purified by preparative LCMS to give the title compound as white solid (11.14%). LCMS m/z=602.6 [M+H]⁺. ¹H NMR (400 MHz, CD₃OD) δ ppm 2.85 (m, 2H), 3.25 (m, 4H), 3.56 (m, 6H), 4.72 (s, 2H), 4.91 (s, 2H), 7.26 (dd, J₁=8.6 Hz, J₂=2.0 Hz, 1H), 7.35-7.40 (m, 2H), 7.72 (dd, J₁=J₂=7.1 Hz, 1H), 8.30 (d, J=8.6 Hz, 1H).

### Example 1.389

### Preparation of (S)-tert-Butyl 2-(4-(4-Chloro-2-(4-(3,3,3-trifluoropropyl)piperazin-1-yl)phenylcarbamoyl)-3-fluorophenylcarbamoyl)morpholine-4-carboxylate (Compound 462) and (S)—N-(4-(4-Chloro-2-(4-(3,3,3-trifluoropropyl)piperazin-1-yl)phenylcarbamoyl)-3-fluorophenyl)morpholine-2-carboxamide (Compound 300)

### Step A: Preparation of 4-Amino-N-(4-chloro-2-(4-(3,3,3-trifluoropropyl)piperazin-1-yl)phenyl)-2-fluorobenzamide

A mixture of 4-chloro-2-(4-(3,3,3-trifluoropropyl)piperazin-1-yl)aniline (0.105 g, 0.341 mmol), 4-amino-2-fluorobenzoic acid (0.053 g, 0.341 mmol), HATU (0.130 g, 0.341 mmol) and TEA (0.048 mL, 0.341 mmol) was taken up in DMF (2 mL) and heated to 50° C. for 18 h in a 5 mL sealed scintillation vial. The crude product was purified by preparative LCMS to give the title compound as white solid (56%). LCMS m/z=445.4 [M+H]⁺.

### Step B: Preparation of (S)—N-(4-(4-Chloro-2-(4-(3,3,3-trifluoropropyl)piperazin-1-yl)phenylcarbamoyl)-3-fluorophenyl)morpholine-2-carboxamide (Compound 300)

A mixture of 4-amino-N-(4-chloro-2-(4-(3,3,3-trifluoropropyl)piperazin-1-yl)phenyl)-2-fluorobenzamide (40 mg, 0.090 mmol), (S)-4-(tert-butoxycarbonyl)morpholine-2-carboxylic acid (20.79 mg, 0.090 mmol), HATU (51.3 mg, 0.135 mmol) and TEA (0.038 mL, 0.270 mmol) was taken up in DMF (1 mL) and heated to 50° C. for 18 h in a 5 mL sealed scintillation vial. The crude product was purified by preparative LCMS to give Compound 462. To a solution of Compound 462 in 0.5 mL acetonitrile was added 0.2 mL HCl solution in 1,4-dioxane at room temperature. The mixture was stirred at room temperature for 4 h, then it was concentrated under reduced pressure. The residue was purified by preparative LCMS to give Compound 300 as white solid (10.9%). LCMS m/z=558.4 [M+H]⁺; ¹H NMR (400 MHz, CD₃OD) δ ppm 2.88 (m, 2H), 3.27 (m, 5H), 3.29 (m, 1H), 3.44 (d, J=13.2 Hz, 2H), 3.51 (m, 5H), 3.74 (dd, J₁=12.9 Hz, J₂=2.8 Hz, 1H), 4.34 (t, J=12.9 Hz, 1H), 4.58 (dd, J₁=10.4 Hz, J₂=3.1 Hz, 1H), 7.33 (dd, J₁=8.6 Hz, J₂=2.5 Hz, 1H), 7.44 (d, J₁=2.2 Hz, 1H), 7.58 (dd, J₁=8.4 Hz, J₂=2.0 Hz, 1H), 8.11 (dd, J₁=J₂=9.0 Hz, 1H), 8.48 (d, J=8.9 Hz, 1H).

### Example 1.390

### Preparation of Sodium Salt of 2-(4-(4-Chloro-2-(4-(3,3,3-trifluoropropyl)piperazin-1-yl)phenylcarbamoyl)-2,3-difluorobenzylamino)-2-oxoethyl dihydrogen Phosphate (Compound 382)

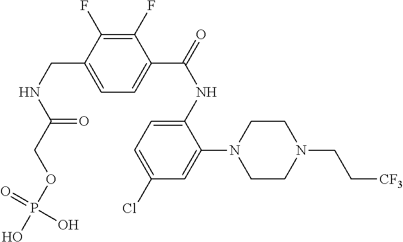 [SMILES: O=C(COP(=O)(O)O)NCc1ccc(C(=O)Nc2ccc(Cl)cc2N2CCN(CCC(F)(F)F)CC2)c(F)c1F]

### Step A: Preparation of N-(4-Chloro-2-(4-(3,3,3-trifluoropropyl)piperazin-1-yl)phenyl)-2,3-difluoro-4-((2-hydroxyacetamido)methyl)benzamide

Intermediate 3 (200 mg, 0.419 mmol), 2-hydroxyacetic acid (35.1 mg, 0.461 mmol), DIEA (110 μL, 0.629 mmol), and HATU (191 mg, 0.503 mmol) were added to a vial with DMF (3 mL). The reaction was stirred at 50° C. for an hour. The reaction was cooled and extracted with H₂O and EtOAc (3×5 mL). The organic layers were combined, dried, and concentrated. The residue was purified by column chromatography to give the title compound (200 mg). The obtained material (40 mg of which) was dissolved in ACN (0.4 mL), added HCl (5M in H₂O; 1 eq.) and H₂O (0.4 mL), frozen, and lyophilized to give the HCl salt of the title compound (42 mg, 94%). LCMS m/z=535.4 [M+H]⁺; ¹H NMR (400 MHz, DMSO-d₆) δ ppm 2.84-3.04 (m, 3H), 3.05-3.28 (m, 4H), 3.37-3.79 (m, 6H), 3.89 (s, 2H), 4.44 (d, J=6.10 Hz, 2H), 7.23-7.32 (m, 2H), 7.34 (s, 1H), 7.61 (t, J=7.25 Hz, 1H), 8.17 (d, J=8.14 Hz, 1H), 8.44 (t, J=5.98 Hz, 1H), 9.68 (d, J=4.58 Hz, 1H).

### Step B: Preparation of Sodium Salt of 2-(4-(4-Chloro-2-(4-(3,3,3-trifluoropropyl)piperazin-1-yl)phenylcarbamoyl)-2,3-difluorobenzylamino)-2-oxoethyl dihydrogen Phosphate (Compound 382)

N-(4-Chloro-2-(4-(3,3,3-trifluoropropyl)piperazin-1-yl)phenyl)-2,3-difluoro-4-((2-hydroxyacetamido)methyl)benzamide (160 mg, 0.299 mmol, Compound 270, see Example 1.274) was dissolved in THF (2 mL). 1H-tetrazole (58.8 mg, 0.839 mmol) and CH₂Cl₂ (2 mL) were added, followed by di-tert-butyl diisopropylphosphoramidite (349 mg, 1.258 mmol). The reaction was stirred at room temperature for 4 hours. tert-Butyl hydroperoxide (203 μL, 2.097 mmol) was added. The reaction was stirred at room temperature overnight. The mixture was purified by column chromatography to give an intermediate phosphate ester (145 mg). The obtained material was dissolved in CH₂Cl₂ (3 mL) and added TFA (500 μL, 6.49 mmol). The reaction was stirred at room temperature overnight. The next day, the solvent was removed and the residue was purified by HPLC. Some obtained material (60 mg) was converted to its corresponding sodium salt by dissolving the material in H₂O and NaOH (6 eq). The material was then subject to a C18 reverse phase column (5% MeOH/H₂β isocratic) to give the sodium salt of the title compound (51 mg, 18.46%). LCMS m/z=615.2 [M+H]⁺; ¹H NMR (400 MHz, D₂O) δ ppm 2.45-2.63 (m, 2H), 2.71-2.87 (m, 6H), 2.98-3.09 (m, 4H), 4.38 (d, J=6.61 Hz, 2H), 4.68 (s, 2H), 7.28 (dd, J=8.90, 1.78 Hz, 1H), 7.38-7.44 (m, 2H), 7.67 (t, J=7.25 Hz, 1H), 7.91 (d, J=8.65 Hz, 1H).

### Example 1.391

### Preparation of Sodium Salt of (S)-1-(4-(4-Chloro-2-(4-(3,3,3-trifluoropropyl)piperazin-1-yl)phenylcarbamoyl)-2,3-difluorobenzylcarbamoyl)pyrrolidin-3-yl Dihydrogen Phosphate (Sodium Salt of Compound 440)

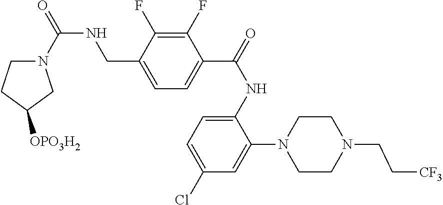

To a solution of (S)—N-(4-(4-chloro-2-(4-(3,3,3-trifluoropropyl)piperazin-1-yl)phenylcarbamoyl)-2,3-difluorobenzyl)-3-hydroxypyrrolidine-1-carboxamide (200 mg, 0.34 mmol) in THF (2 mL), was added di-tert-butyl diisopropylphosphoramidite (188 mg, 0.678 mmol) followed by 1H-tetrazole (71.2 mg, 1.02 mmol) at room temperature. After stirring for 5 h, the reaction was treated with 70% aqueous 2-hydroperoxy-2-methylpropane (87.1 mL, 0.68 mmol) and stirred for 2 h. The reaction was extracted with ethyl acetate, dried over MgSO₄, and concentrated under reduced pressure. The resulting residue was dissolved in acetonitrile (0.3 mL) and treated with NaOH (133.2 mg, 3.42 mmol) in water (1 mL) at room temperature. The reaction solution was directly purified by C-18 reverse phase column chromatography (5% acetonitrile/water) to give the title compound (85 mg, 35.1%). LCMS m/z=670.1[M+H]⁺; NMR (400 M Hz, DMSO-d₆) δ ppm 1.75-1.90 (m, 1H), 2.91-3.72 (m, 16H), 4.12 (m, 1H), 4.35 (d, J=5.5 Hz, 2H), 6.85-6.92 (m, 1H), 7.25-7.41 (m, 3H), 7.69 (m, 1H), 8.21 (d, J=8.4 Hz, 1H), 9.52 (d, J=4.7 Hz, 1H), 11.4 (br, 1H).

### Example 1.392

### Preparation of N-(4-(4,5-Difluoro-2-(4-(3,3,3-trifluoropropyl)piperazin-1-yl)phenylcarbamoyl)-2,3-difluorobenzyl)-4-(hydroxymethyl)piperidine-1-carboxamide (Compound 434)

### Step A: Preparation of 1-(4,5-difluoro-2-nitrophenyl)-4-(3,3,3-trifluoropropyl)piperazine

1,2,4-Trifluoro-5-nitrobenzene (1 g, 5.65 mmol) was dissolved in IPA (20 mL) and cooled in an ice bath. Then, 1-(3,3,3-trifluoropropyl)piperazine dihydrochloride (1.441 g, 5.65 mmol) in IPA (10 mL) and DIEA (3.95 mL, 22.59 mmol) was added slowly to the solution via an addition funnel. Upon complete addition, the reaction was warmed to room temperature and heated at 80° C. in an oil bath for 1 h. The solvent was then evaporated and the residue was partitioned between H₂O and EtOAc (2×100 mL). The organic layers were combined, concentrated and dried to give the title compound (1.5 g). LCMS m/z=340.2 [M+H]⁺.

### Step B: Preparation of 4,5-Difluoro-2-(4-(3,3,3-trifluoropropyl)piperazin-1-yl)aniline

1-(4,5-Difluoro-2-nitrophenyl)-4-(3,3,3-trifluoropropyl)piperazine (1.52 g, 4.48 mmol) was dissolved in EtOH (20 mL). The solution was cooled in an ice bath and SnCl₂ (2.55 g, 13.44 mmol) was added to the reaction. The reaction was warmed to room temperature and then heated in an oil bath at 80° C. for 1 h. After this time, the reaction was cooled in an ice bath and quenched by the slow addition of H₂O (˜10 mL; exothermic). Concentrated NaOH (50 wt %, ˜15 mL) was added via pipette, along with DCM (100 mL) and H₂O (85 mL), until the solids were mostly dissolved and the mixture was stirring vigorously. The reaction was partitioned between NaOH/H₂O (150 mL) and DCM (150 mL) and the organic layer was removed. The aqueous layer was extracted with more DCM (2×150 mL). The organic layers were combined, dried, concentrated, and the residue was purified by column chromatography (0-30% EtOAc/hexanes) to give the title compound (1.38 g). LCMS m/z=310.4 [M+H]⁺; ¹H NMR (400 M Hz, DMSO-d₆) δ ppm 2.51-2.61 (m, 8H), 2.81-2.86 (m, 4H), 4.95 (s, 2H), 6.54 (dd, J=13.64, 8.46 Hz, 1H), 6.75 (dd, J=12.82, 8.02 Hz, 1H).

### Step C: Preparation of 2,3-Difluoro-4-((4-(hydroxymethyl)piperidine-1-carboxamido)methyl)benzoic Acid

Ethyl 4-(aminomethyl)-2,3-difluorobenzoate (1.8 g, 8.36 mmol) was dissolved in DMF (10 mL) and cooled in an ice bath. Bis(2,5-dioxopyrrolidin-1-yl) carbonate (2.357 g, 9.20 mmol), pre-dissolved in DMF (5 mL), was added quickly to the solution on ice. After complete addition, the reaction was warmed to room temperature. Piperidin-4-ylmethanol (1.156 g, 10.04 mmol) was added to the solution. The reaction was heated to 60° C. in an oil bath and stirred at this temperature overnight. An extraction was performed under acidic conditions (100 mL each of 1 M HCl/H₂O/Brine and EtOAc). The organic layers were combined, dried and concentrated to yield the crude ester intermediate. This intermediate was dissolved in THF (10 mL). LiOH (0.601 g, 25.09 mmol) was added along with H₂O (10 mL). The solution was stirred with mixing until the ester cleavage was complete (˜2 h). Then the solution was cooled in an ice bath and made acidic by addition of 5 M HCl. The reaction was extracted with 100 mL each of HCl, H₂O, brine and EtOAc. The organic layers were combined and dried to give the title compound (2.2 g). LCMS m/z=329.2 [M+H]⁺; ¹H NMR (400 M Hz, DMSO-d₆) δ ppm 0.99 (dd, J=11.87, 3.66 Hz, 2H), 1.46-1.56 (m, J=10.61, 4.29 Hz, 1H), 1.61 (d, J=12.88 Hz, 2H), 2.61-2.72 (m, 2H), 3.22-3.28 (m, 2H), 3.97 (d, J=13.14 Hz, 2H), 4.31 (t, J=5.68 Hz, 2H), 4.40-4.46 (m, 1H), 7.09 (t, J=5.62 Hz, 1H), 7.14-7.21 (m, 1H), 7.60-7.66 (m, 1H), 13.43 (bs, 1H).

### Step D: Preparation of N-(4-(4,5-Difluoro-2-(4-(3,3,3-trifluoropropyl)piperazin-1-yl)phenylcarbamoyl)-2,3-difluorobenzyl)-4-(hydroxymethyl)piperidine-1-carboxamide (Compound 434)

4,5-Difluoro-2-(4-(3,3,3-trifluoropropyl)piperazin-1-yl)aniline (10 mg, 0.024 mmol), 2,3-difluoro-4-((4-(hydroxymethyl)piperidine-1-carboxamido)methyl)benzoic acid (7.76 mg, 0.024 mmol), DIEA (30 μL, 0.172 mmol), and HATU (10.78 mg, 0.028 mmol) were dissolved in DMF (0.3 mL). The reaction was heated with stirring to 100° C. for 1 h. The reaction mixture was purified by preparative LC/MS (5-70% ACN/H₂O (0.1% TFA), 25 min) to give the title compound as a mixture (˜60/40) with the TFA ester, (1-(4-(4,5-difluoro-2-(4-(3,3,3-trifluoropropyl)piperazin-1-yl)phenylcarbamoyl)-2,3-difluorobenzylcarbamoyl)piperidin-4-yl)methyl 2,2,2-trifluoroacetate. LCMS m/z=620.6 [M+H]⁺ (for Compound 434) and LCMS m/z=716.8 [M+H]⁺ (for the TFA ester).

### Example 1.393

### Preparation of 4-(Aminomethyl)-N-(4-chloro-2-(4-(3,3,3-trifluoropropyl)piperazin-1-yl)phenyl)-2-fluorobenzamide (Intermediate 4)

### Step A: Preparation of 4-Chloro-2-(4-(3,3,3-trifluoropropyl)piperazin-1-yl)aniline

Piperazine (36.8 g, 427 mmol) was dissolved in IPA (150 mL) and the resulting solution was cooled in an ice bath. 4-Chloro-2-fluoro-1-nitrobenzene (25 g, 142 mmol), pre-dissolved in IPA (100 mL), was added to the solution slowly via an addition funnel. Upon completion of the addition, the reaction was warmed to room temperature and stirred overnight. The next day, the solvent was evaporated and the residue was partitioned between H₂O (200 mL) and EtOAc (200 mL). The aqueous layer was further extracted with EtOAc (2×200 mL). The organic layers were combined, and washed with H₂O/brine (500 mL). The organic layer was dried over MgSO₄ and concentrated to yield 1-(5-chloro-2-nitrophenyl)piperazine as a reddish oil. This material was dissolved in THF (50 mL) and MeOH (10 mL) and diisopropylethylamine (DIEA) (49.7 mL, 285 mmol) and 3-bromo-1,1,1-trifluoropropane (22.84 mL, 214 mmol) were added. The reaction was refluxed overnight. The next day, the reaction was around 70% complete. Thus, more 3-bromo-1,1,1-trifluoropropane (10 mL) and DIEA (20 mL) were added and the reaction was again heated to reflux overnight. The solvent was evaporated to yield 1-(5-chloro-2-nitrophenyl)-4-(3,3,3-trifluoropropyl)piperazine, as a reddish-yellow waxy solid. The solid was dissolved in EtOH (150 mL) and the reaction was cooled in an ice bath. To the stirring solution was added SnCl₂ (81 g, 427 mmol) portionwise (in 10 g portions; allowing the tin chloride to fully dissolve and the reaction to cool in between). Upon completion of the addition, the reaction was heated at 80° C. for 1 h. The reaction was cooled in an ice bath and aqueous NaOH (50 wt %) was added portionwise (in ˜20 mL portions). DCM, and H₂O were added (enough to sufficiently dissolve the tin chloride and form two layers separable in a separation funnel; ˜1.2 L each). The organic layer was removed and the aqueous layer was extracted with DCM (2×1 L). The organic layers were combined, dried, and concentrated. The residue was purified by column chromatography to give the title compound as a light-yellow/tan solid (42.7 g). LCMS m/z=308.2 [M+H]⁺; ¹H NMR (400 M Hz, DMSO-d₆) δ ppm 2.43-2.66 (m, 8H) 2.76-2.89 (m, 4H) 4.81-4.82 (m, 2H) 6.69 (d, T=8.21 Hz, 1H) 6.82-6.89 (m, 2H).

### Step B: Preparation of N-(4-Chloro-2-(4-(3,3,3-trifluoropropyl)piperazin-1-yl)phenyl)-4-cyano-2-fluorobenzamide

To a suspension of 4-cyano-2-fluorobenzoic acid (0.537 g, 3.25 mmol) in DCM (10 mL), was added oxalyl chloride (1.422 mL, 16.25 mmol) followed by few drops of DMF. After stirring for 2 h, the reaction was concentrated under reduced pressure. The residue was dissolved in fresh DCM (10 mL) and treated with 4-chloro-2-(4-(3,3,3-trifluoropropyl)piperazin-1-yl)aniline (1.0 g, 3.25 mmol) followed by triethylamine (0.329 g, 3.25 mmol) at an ambient temperature. After stirring for 1 h, the reaction was washed with water, dried over MgSO₄, and then concentrated under reduced pressure. The residue was triturated with methanol and filtered to give the title compound (1.25 g). LCMS m/z=455.3[M+H]⁺.

### Step C: Preparation of 4-(Aminomethyl)-N-(4-Chloro-2-(4-(3,3,3-trifluoropropyl)piperazin-1-yl)phenyl)-2-fluorobenzamide (Intermediate 4)

To a suspension of N-(4-chloro-2-(4-(3,3,3-trifluoropropyl)piperazin-1-yl)phenyl)-4-cyano-2-fluorobenzamide (0.1 g, 0.220 mmol) and cobalt(II) chloride hexahydrate (0.105 g, 0.440 mmol) in methanol (2 mL), was added NaBH₄ (0.083 g, 2.199 mmol) at 0° C. After stirring for 1 h at room temperature, the reaction was concentrated under reduced pressure. The residue was quenched with 2 M HCl and washed with ether. The aqueous layer was basified with 1 M NaOH, and then extracted with DCM. The organic layer was dried over MgSO₄ and concentrated under reduced pressure. The residue was purified by column chromatography to give the title compound (0.056 g). LCMS m/z=459.2[M+H]⁺.

### Example 1.394

### Preparation of Sodium Salt of (R)-1-(4-(4-Chloro-2-(4-(3,3,3-trifluoropropyl)piperazin-1-yl)phenylcarbamoyl)-2,3-difluorobenzylcarbamoyl)pyrrolidin-3-yl Dihydrogen Phosphate (Compound 441)

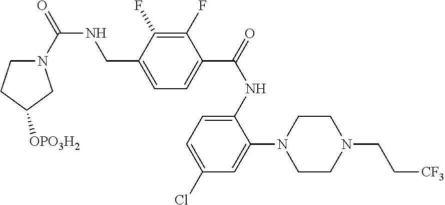

To a solution of (R)—N-(4-(4-chloro-2-(4-(3,3,3-trifluoropropyl)piperazin-1-yl)phenylcarbamoyl)-2,3-difluorobenzyl)-3-hydroxypyrrolidine-1-carboxamide hydrochloride (200 mg, 0.319 mmol) in CH₂Cl₂ (2 mL), was added di-tert-butyl diisopropylphosphoramidite (443 mg, 1.596 mmol) followed by 1H-tetrazole (112 mg, 1.596 mmol) and triethylamine (32.3 mg, 0.319 mmol) at room temperature. After stirring for 2 hr, 2-hydroperoxy-2-methylpropane (57.5 mg, 0.639 mmol) was added to the reaction and stirred for 1 hr. The reaction was extracted with ethyl acetate, dried over MgSO₄, and concentrated under vacuum. The resulting residue was purified by column chromatography. The resulting t-butyl phosphate intermediate was treated with 50% TFA in CH₂Cl₂ (5 mL). After 5 hrs, the reaction was concentrated under vacuum and the resulting residue was dissolved in acetonitrile (1 mL) and 2.0 M aqueous NaOH (3 mL) and then purified by C18 reverse phased column chromatography (10% acetonitrile in H₂O) to give sodium (R)-1-(4-(4-chloro-2-(4-(3,3,3-trifluoropropyl)piperazin-1-yl)phenylcarbamoyl)-2,3-difluorobenzylcarbamoyl)pyrrolidin-3-yl phosphate (125 mg, 54.8%). LCMS m/z=669.5 [M+H]⁺.

### Example 1.395

### Preparation of N-(4-(4-Chloro-2-(4-(3,3,3-trifluoropropyl)piperazin-1-yl)phenylcarbamoyl)-3-fluorobenzyl)piperazine-1-carboxamide (Compound 394)

To a solution of Intermediate 4 (15 mg, 0.033 mmol) in DMF (1 mL) was added bis(2,5-dioxopyrrolidin-1-yl) carbonate (8.45 mg, 0.033 mmol). After stirring for 30 min, piperazine (5.63 mg, 0.065 mmol) was added the reaction was heated to 100° C. for 30 min. The reaction mixture was poured into water and extracted with ethyl acetate. The organic layer was dried over MgSO₄ and concentrated under reduced pressure to give a residue, which was purified by HPLC to give the title compound (12 mg). LCMS m/z=571.6 [M+H]⁺.

### Examples 1.396 to 1.401

The following compounds were prepared using the disclosed intermediates and a method similar to the one described in Example 1.395.

| | | | Product |
|---|---|---|---|
| Example | Cmpd | | (Mass Observed |
| No. | No. | Intermediates Used | LC/MS m/z) |
| 1.396 | 396 | Intermediate 4 and | 620.4 [M + H]+ |
| | | 1,1-dioxo-thiomorpholine | |
| 1.397 | 397 | Intermediate 4 and | 572.4 [M + H]+ |
| | | (S)-pyrrolidin-3-ol | |
| 1.398 | 398 ¹ | Intermediate 4 and | 615.4 [M + H]+ |
| | | 2-(piperazin-1-yl)ethanol | |
| 1.399 | 399 | Intermediate 4 and | 600.4 [M + H]+ |
| | | piperidin-4-ylmethanol | |
| 1.400 | 401 ² | Intermediate 4 and | 558.4 [M + H]+ |
| | | azetidin-3-ol | |
| 1.401 | 395 | Intermediate 4 and | 599.5 [M + H]+ |
| | | N-ethylpiperidine | |

¹ Compound 398: ¹H NMR (400 M Hz, DMSO-d₆) ppm 9.68 (d, J = 8.1 Hz, 1H), 8.34 (d, J = 8.1 Hz, 1H). 7.91(m, 1H), 7.52 (s, 1H), 7.27-7.32 (m, 3H), 4.37 (m, 2H), 4.10-4.23 (br, 4H), 3.78 (m, 2H), 2.92-3.51 (br, 20H).
² Compound 401: ¹H NMR (400 M Hz, DMSO-d₆) ppm 3.10-3.32 (br, 11H), 3.51-3.54 (br, 2H), 3.91-3.93 (br, 2H), 4.21 (br, 2H), 4.35 (m, 2H), 6.92 (m, 1H), 7.14-7.24 (m, 4H), 7.31 (s, 1H), 7.82 (m, 1H), 8.25 (d, J = 8.1 Hz, 1H), 9.57 (d, J = 8.1 Hz, 1H).

### Example 1.402

### Preparation of (S)-tert-Butyl 1-(4-(4-Chloro-2-(4-(3,3,3-trifluoropropyl)piperazin-1-yl)phenylcarbamoyl)-3-fluorobenzylcarbamoyl)piperidin-3-ylcarbamate (Compound 442) and (S)-3-Amino-N-(4-(4-Chloro-2-(4-(3,3,3-trifluoropropyl)piperazin-1-yl)phenylcarbamoyl)-3-fluorobenzyl)piperidine-1-carboxamide (Compound 400)

To a solution of Intermediate 4 (15 mg, 0.033 mmol) in DMF (2 mL), was added bis(2,5-dioxopyrrolidin-1-yl) carbonate (8.37 mg, 0.033 mmol). After stirring for 30 min, the reaction was treated with (S)-tert-butyl piperidin-3-ylcarbamate (6.55 mg, 0.033 mmol). The reaction was heated to 50° C. for 30 min. After cooling to room temperature, the mixture was extracted with ethyl acetate and the organic extracts were concentrated under reduced pressure to provide Compound 484. The resulting compound was treated with 4.0 M HCl in dioxane (2 mL) and stirred overnight. The mixture was concentrated under reduced pressure and purified by HPLC to give the title compound (8.7 mg). Compound 400: LCMS m/z=585.4 [M+H]⁺.

### Examples 1.403 and 1.404

The following compounds were prepared using the disclosed intermediates and a method similar to the one described in Example 1.402.

| | | | Product |
|---|---|---|---|
| Example | Cmpd | | (Mass Observed |
| No. | No. | Intermediates Used | LC/MS m/z) |
| 1.403 | 383 | Intermediate 4 and (R)- | N.D. ¹ |
| | | tert-butyl pyrrolidin- | |
| | | 3-ylcarbamate | |
| | 402 | Deprotection | 571.2 [M + H]⁺ |
| 1.404 | 384 | Intermediate 4 and (S)- | N.D. ¹ |
| | | tert-butyl pyrrolidin- | |
| | | 3-ylcarbamate | |
| | 403 | Deprotection | 571.5 [M + H]⁺ |

¹ Not Determined

### Example 1.405

### Preparation of N-(4-(4-Chloro-2-(4-(3,3,3-trifluoropropyl)piperazin-1-yl)phenylcarbamoyl)-2,3-difluorobenzyl)piperazine-1-carboxamide (Compound 404)

### Step A: Preparation of 2,5-Dioxopyrrolidin-1-yl 4-(4-Chloro-2-(4-(3,3,3-trifluoropropyl)piperazin-1-yl)phenylcarbamoyl)-2,3-difluorobenzylcarbamate (Intermediate 5)

To a solution of Intermediate 3 (1.05 g, 2.202 mmol) in DMF (1 mL), was added bis(2,5-dioxopyrrolidin-1-yl) carbonate (0.564 g, 2.202 mmol) at room temperature. The reaction was stirred for 1 h to provide a solution of Intermediate 5. The solution containing Intermediate 5 was used in the next step without further purification.

### Step B: Preparation of N-(4-(4-Chloro-2-(4-(3,3,3-trifluoropropyl)piperazin-1-yl)phenylcarbamoyl)-2,3-difluorobenzyl)piperazine-1-carboxamide (Compound 404)

To a solution of Intermediate 5 (25 mg, 0.040 mmol) in DMF (1 mL), was added piperazine (3.48 mg, 0.040 mmol), followed by few drops of DIEA at room temperature. The reaction was heated at 50° C. for 1 h. After cooling, the precipitate was filtered off and purified by HPLC to give the title compound. LCMS m/z=589.6 [M+H]⁺.

### Examples 1.406, 1.410 to 1.412, 1.415, and 1.418 to 1.420

The following compounds were prepared using the disclosed intermediates and a method similar to the one described in Example 1.405, Step B.

| | | | Product |
|---|---|---|---|
| Example | Cmpd | | (Mass Observed |
| No. | No. | Intermediates Used | LC/MS m/z) |
| 1.406 | 405 | Intermediate 5 and | 633.7 [M + H]⁺ |
| | | 2-(piperazin-1- | |
| | | yl)ethanol | |
| 1.410 | 409 | Intermediate 5 and | 631.9 [M + H]⁺ |
| | | N,N-dimethyl- | |
| | | piperidin-4-amine | |
| 1.411 | 410 | Intermediate 5 and | 638.6 [M + H]⁺ |
| | | 1,1-dioxo- | |
| | | thiomorpholine | |
| 1.412 | 411 | Intermediate 5 and | 632.5 [M + H]⁺ |
| | | piperazine-1- | |
| | | carboxamide | |
| 1.415 | 414 | Intermediate 5 and | 632.7 [M + H]⁺ |
| | | 2-(piperidin-2- | |
| | | yl)ethanol | |
| 1.418 | 418 | Intermediate 5 and | 604.4 [M + H]⁺ |
| | | azetidine-3- | |
| | | carboxylic acid | |
| 1.419 | 419 | Intermediate 5 and | 620.4 [M + H]⁺ |
| | | (R)-morpholin-2- | |
| | | ylmethanol | |
| 1.420 | 435 | Intermediate 5 and | 618.4 [M + H]⁺ |
| | | (R)-piperidin-3- | |
| | | ylmethanol | |

### Example 1.407

### Preparation of (S)—N-(4-(4-Chloro-2-(4-(3,3,3-trifluoropropyl)piperazin-1-yl)phenylcarbamoyl)-2,3-difluorobenzyl)-3-hydroxypyrrolidine-1-carboxamide (Compound 406)

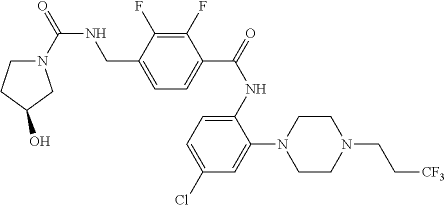 [SMILES: O=C(Nc1ccc(Cl)cc1N1CCN(CCC(F)(F)F)CC1)c1ccc(CNC(=O)N2CC[C@H](O)C2)c(F)c1F]

From Intermediate 5 and (S)-pyrrolidin-3-ol, the title compound was obtained using a method similar to the one described in Example 1.405, Step B. LCMS m/z=590.5 [M+H]⁺; ¹H NMR (400 M Hz, DMSO-d₆) ppm 1.62-1.89 (m, 2H), 2.95-3.14 (br, 2H), 3.01-3.85 (br, 15H), 4.32 (br, 1H), 4.38 (s, 2H), 6.75 (m, 1H), 7.15-7.22 (m, 2H), 7.31 (s, 1H), 7.60 (m, 1H), 8.25 (d, J=8.4 Hz, 1H), 9.69 (d, J=8.1 Hz, 1H).

### Example 1.408

### Preparation of N-(4-(4-Chloro-2-(4-(3,3,3-trifluoropropyl)piperazin-1-yl)phenylcarbamoyl)-2,3-difluorobenzyl)-4-(hydroxymethyl)piperidine-1-carboxamide (Compound 407)

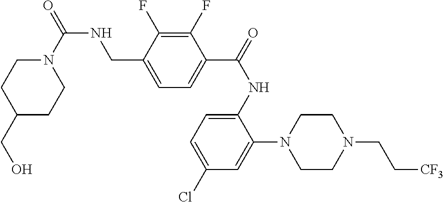 [SMILES: O=C(Nc1ccc(Cl)cc1N1CCN(CCC(F)(F)F)CC1)c1ccc(CNC(=O)N2CCC(CO)CC2)c(F)c1F]

From Intermediate 5 and piperidin-4-ylmethanol, the title compound was obtained using a method similar to the one described in Example 1.405, Step B. LCMS m/z=618.4 [M+H]⁺; ¹H NMR (400 M Hz, DMSO-d₆) ppm 0.98-1.14 (m, 2H), 1.52-1.79 (m, 3H), 2.72-2.85 (m, 2H), 3.15-3.81 (br, 16H), 3.91-3.42 (m, 2H), 4.35 (d, J=8.4 Hz, 2H), 7.15-7.41 (m, 3H), 7.75 (m, 1H), 8.21 (d, J=8.7 Hz, 1H), 9.73 (d, J=8.4 Hz, 1H).

### Example 1.409

### Preparation of (S)—N¹-(4-(4-Chloro-2-(4-(3,3,3-trifluoropropyl)piperazin-1-yl)phenylcarbamoyl)-2,3-difluorobenzyl)pyrrolidine-1,2-dicarboxamide (Compound 408)

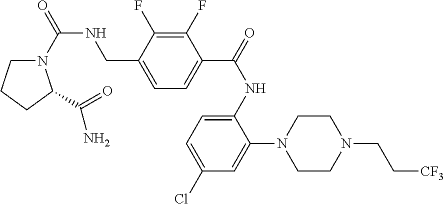 [SMILES: NC(=O)[C@@H]1CCCN1C(=O)NCc1ccc(C(=O)Nc2ccc(Cl)cc2N2CCN(CCC(F)(F)F)CC2)c(F)c1F]

From Intermediate 5 and (S)-pyrrolidine-2-carboxamide, the title compound was obtained using a method similar to the one described in Example 1.405, Step B. LCMS m/z=617.5 [M+H]⁺.

### Example 1.413

### Preparation of N-(4-(4-Chloro-2-(4-(3,3,3-trifluoropropyl)piperazin-1-yl)phenylcarbamoyl)-2,3-difluorobenzyl)-3-oxopiperazine-1-carboxamide (Compound 412)

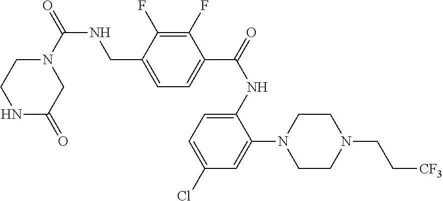 [SMILES: O=C1CN(C(=O)NCc2ccc(C(=O)Nc3ccc(Cl)cc3N3CCN(CCC(F)(F)F)CC3)c(F)c2F)CCN1]

From Intermediate 5 and piperazin-2-one, the title compound was obtained using a method similar to the one described in Example 1.405, Step B. LCMS m/z=603.5 [M+H]⁺.

### Example 1.414

### Preparation of (R)—N-(4-(4-Chloro-2-(4-(3,3,3-trifluoropropyl)piperazin-1-yl)phenylcarbamoyl)-2,3-difluorobenzyl)-2-(hydroxymethyl)pyrrolidine-1-carboxamide (Compound 413)

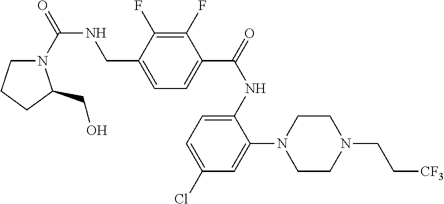 [SMILES: O=C(Nc1ccc(Cl)cc1N1CCN(CCC(F)(F)F)CC1)c1ccc(CNC(=O)N2CCC[C@@H]2CO)c(F)c1F]

From Intermediate 5 and (R)-pyrrolidin-2-ylmethanol, the title compound was obtained using a method similar to the one described in Example 1.405, Step B. LCMS m/z=604.5 [M+H]⁺.

### Example 1.416

### Preparation of N-(4-(4-Chloro-2-(4-(3,3,3-trifluoropropyl)piperazin-1-yl)phenylcarbamoyl)-2,3-difluorobenzyl)-3-hydroxyazetidine-1-carboxamide (Compound 415)

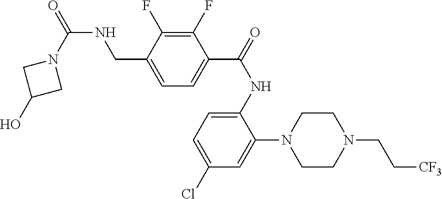 [SMILES: O=C(Nc1ccc(Cl)cc1N1CCN(CCC(F)(F)F)CC1)c1ccc(CNC(=O)N2CC(O)C2)c(F)c1F]

From Intermediate 5 and azetidin-3-ol, the title compound was obtained using a method similar to the one described in Example 1.405, Step B. LCMS m/z=576.6 [M+H]⁺.

### Example 1.417

### Preparation of N-(4-(4-Chloro-2-(4-(3,3,3-trifluoropropyl)piperazin-1-yl)phenylcarbamoyl)-2,3-difluorobenzyl)-2-(hydroxymethyl)morpholine-4-carboxamide (Compound 416)

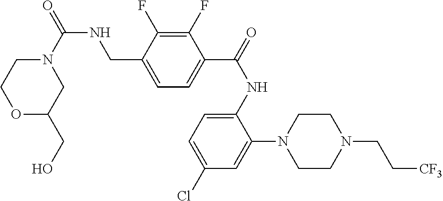 [SMILES: O=C(Nc1ccc(Cl)cc1N1CCN(CCC(F)(F)F)CC1)c1ccc(CNC(=O)N2CCOC(CO)C2)c(F)c1F]

From Intermediate 5 and morpholin-2-ylmethanol, the title compound was obtained using a method similar to the one described in Example 1.405, Step B. LCMS m/z=620.6 [M+H]⁺.

### Example 1.421

### Preparation of (R)—N-(4-(4-Chloro-2-(4-(3,3,3-trifluoropropyl)piperazin-1-yl)phenylcarbamoyl)-2,3-difluorobenzyl)-3-hydroxypiperidine-1-carboxamide (Compound 436)

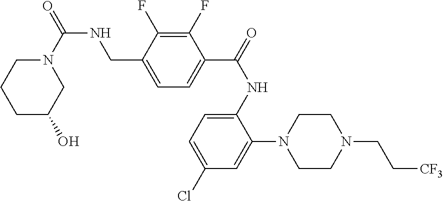 [SMILES: O=C(Nc1ccc(Cl)cc1N1CCN(CCC(F)(F)F)CC1)c1ccc(CNC(=O)N2CCC[C@@H](O)C2)c(F)c1F]

From Intermediate 5 and (R)-piperidin-3-ol, the title compound was obtained using a method similar to the one described in Example 1.405, Step B. LCMS m/z=604.4 [M+H]⁺.

### Example 1.422

### Preparation of (S)—N-(4-(4-Chloro-2-(4-(3,3,3-trifluoropropyl)piperazin-1-yl)phenylcarbamoyl)-2,3-difluorobenzyl)-3-hydroxypiperidine-1-carboxamide (Compound 437)

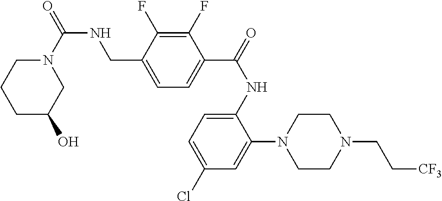 [SMILES: O=C(Nc1ccc(Cl)cc1N1CCN(CCC(F)(F)F)CC1)c1ccc(CNC(=O)N2CCC[C@H](O)C2)c(F)c1F]

From Intermediate 5 and (S)-piperidin-3-ol, the title compound was obtained using a method similar to the one described in Example 1.405, Step B. LCMS m/z=604.4 [M+H]⁺.

### Example 1.423

### Preparation of (S)—N-(4-(4-Chloro-2-(4-(3,3,3-trifluoropropyl)piperazin-1-yl)phenylcarbamoyl)-2,3-difluorobenzyl)-2-(hydroxymethyl)pyrrolidine-1-carboxamide (Compound 438)

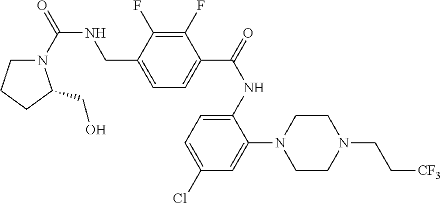 [SMILES: O=C(Nc1ccc(Cl)cc1N1CCN(CCC(F)(F)F)CC1)c1ccc(CNC(=O)N2CCC[C@H]2CO)c(F)c1F]

From Intermediate 5 and (S)-pyrrolidin-2-yl-methanol, the title compound was obtained using a method similar to the one described in Example 1.405, Step B. LCMS m/z=604.6 [M+H]⁺.

### Example 1.424

### Preparation of (R)—N-(4-(4-Chloro-2-(4-(3,3,3-trifluoropropyl)piperazin-1-yl)phenylcarbamoyl)-2,3-difluorobenzyl)-3-(hydroxymethyl)pyrrolidine-1-carboxamide (Compound 439)

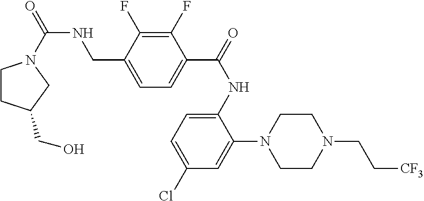 [SMILES: O=C(Nc1ccc(Cl)cc1N1CCN(CCC(F)(F)F)CC1)c1ccc(CNC(=O)N2CC[C@@H](CO)C2)c(F)c1F]

From Intermediate 5 and (R)-pyrrolidin-3-ylmethanol, the title compound was obtained using a method similar to the one described in Example 1.405, Step B. LCMS m/z=604.6 [M+H]⁺.

### Example 1.425

### Preparation of N-(4-(4-Chloro-2-(4-(3,3,3-trifluoropropyl)piperazin-1-yl)phenylcarbamoyl)-3-fluorophenyl)-4-(hydroxymethyl)piperidine-1-carboxamide (Compound 417)

### Step A: Preparation of 4-Amino-N-(4-Chloro-2-(4-(3,3,3-trifluoropropyl)piperazin-1-yl)phenyl)-2-fluorobenzamide

A mixture of 4-amino-2-fluorobenzoic acid (25.2 mg, 0.162 mmol), 4-chloro-2-(4-(3,3,3-trifluoropropyl)piperazin-1-yl)aniline (50 mg, 0.162 mmol), HATU (93 mg, 0.244 mmol) and TEA (0.068 mL, 0.487 mmol) was taken up in DMF (1 mL) and heated to 50° C. for 18 h in a 5 mL sealed scintillation vial. The mixture was purified by preparative LCMS to give the title compound as a brown solid (9.1 mg). LCMS m/z=445.5 [M+H]⁺. ¹H NMR (400 M Hz, CD₃OD) δ ppm 2.83 (m, 2H), 3.20 (m, 4H), 3.52 (m, 6H), 6.46 (dd, J₁=16 Hz, J₂=2 Hz, 1H), 6.55 (dd, J₁=8 Hz, J₂=2 Hz, 1H), 7.23 (dd, J₁=8 Hz, J₂=2 Hz, 1H), 7.34 (d, J=2 Hz, 1H), 7.78 (dd, J₁=J₂=8 Hz, 1H), 8.42 (d, J=8 Hz, 1H).

### Step B: Preparation of N-(4-(4-Chloro-2-(4-(3,3,3-trifluoropropyl)piperazin-1-yl)phenylcarbamoyl)-3-fluorophenyl)-4-(hydroxymethyl)piperidine-1-carboxamide (Compound 417)

To a solution of 4-amino-N-(4-Chloro-2-(4-(3,3,3-trifluoropropyl)piperazin-1-yl)phenyl)-2-fluorobenzamide (20 mg, 0.045 mmol) in DMF (1 mL), was added bis(2,5-dioxopyrrolidin-1-yl)carbonate (11.52 mg, 0.045 mmol). After stirring for 30 min, the reaction was treated with piperidin-4-ylmethanol (5.18 mg, 0.045 mmol). The reaction was heated to 50° C. for 2 h. The mixture was purified by preparative LCMS to give the title compound (2.4 mg) as a brown solid. LCMS m/z=586.7 [M+H]⁺.

### Examples 1.426 to 1.429, and 1.432

The following compounds were prepared using the disclosed intermediates and a method similar to the one described in Example 1.425.

| | | | Product |
|---|---|---|---|
| Example | Cmpd | | (Mass Observed |
| No. | No. | Intermediates Used | LC/MS m/z) |
| 1.426 | 420 ¹ | Intermediate 3 and | 646.6 [M + H]⁺ |
| | | 3-(piperidin-4- | |
| | | yl)propan-1-ol | |
| 1.427 | 421 | Intermediate 3 and | 604.6 [M + H]⁺ |
| | | piperidin-3-ol | |
| 1.428 | 422 | Intermediate 3 and | 613.4 [M + H]⁺ |
| | | piperidine-4-carbonitrile | |
| 1.429 | 423 | Intermediate 3 and | 618.4 [M + H]⁺ |
| | | piperidin-2-ylmethanol | |
| 1.432 | 433 ² | Intermediate 3 and | 618.4 [M + H]⁺ |
| | | (S)-piperidin-3- | |
| | | ylmethanol hydrochloride | |

¹ Compound 420: ¹H NMR (400 M Hz, DMSO-d₆) δ ppm 0.89-1.05 (m, 2H), 1.15-1.33 (m, 2H), 1.34-1.51 (m, 2H), 1.58-1.67 (m, 2H), 1.69-1.79 (m, 1H), 2.66 (t, J = 12.00 Hz, 2H), 2.81-2.98 (m, 2H), 3.04-3.30 (m, 5H), 3.38 (t, J = 6.57 Hz, 2H), 3.44-3.53 (m, 4H), 3.92-4.01 (m, 3H), 4.34 (d, J = 5.31 Hz, 2H), 4.38 (t, J = 6.63 Hz, 1H), 7.15 (q, J = 5.31 Hz, 1H), 7.21-7.31 (m, 2H), 7.36 (d, J = 2.27 Hz, 1H), 7.60 (t, J = 7.14 Hz, 1H), 8.16 (d, J = 8.46 Hz, 1H), 9.67 (d, J = 4.80 Hz, 1H).
² Compound 433: ¹H NMR (400 M Hz, DMSO-d₆) δ ppm 1.03-1.19 (m, 1H), 1.20-1.39 (m, 2H), 1.41-1.54 (m, 1H), 1.59 (d, J = 11.70 Hz, 1H), 1.71 (d, J = 11.19 Hz, 1H), 2.29-2.46 (m, 2H), 2.67-2.77 (m, 1H), 2.78-3.03 (m, 4H), 3.04-3.19 (m, 4H), 3.47-3.75 (m, 5H), 3.83 (d, J = 12.72 Hz, 1H), 3.96 (d, J = 13.22 Hz, 1H), 4.35 (s, 2H), 7.13 (t, J = 5.59 Hz, 1H), 7.21-7.31 (m, 2H), 7.31-7.41 (m, 1H), 7.61 (t, J = 6.99 Hz, 1H), 8.17 (d, J = 8.14 Hz, 1H), 9.67 (d, J = 4.32 Hz, 1H).

### Example 1.430

### Preparation of N-(4-(4-Chloro-2-(4-(3,3,3-trifluoropropyl)piperazin-1-yl)phenylcarbamoyl)-2,3-difluorobenzyl)-4-hydroxypiperidine-1-carboxamide (Compound 424)

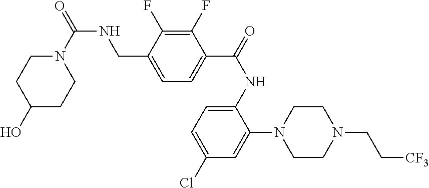 [SMILES: O=C(Nc1ccc(Cl)cc1N1CCN(CCC(F)(F)F)CC1)c1ccc(CNC(=O)N2CCC(O)CC2)c(F)c1F]

From Intermediate 3 and piperidin-4-ol, the title compound was obtained using a method similar to the one described in Example 1.425. LCMS m/z=604.6 [M+H]⁺.

### Example 1.431

### Preparation of (R)—N-(4-(4-Chloro-2-(4-(3,3,3-trifluoropropyl)piperazin-1-yl)phenylcarbamoyl)-2,3-difluorobenzyl)-3-hydroxypyrrolidine-1-carboxamide (Compound 425)

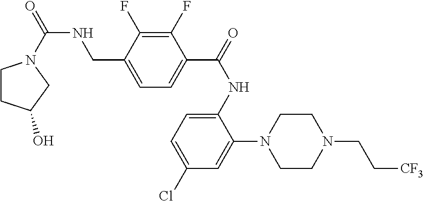 [SMILES: O=C(Nc1ccc(Cl)cc1N1CCN(CCC(F)(F)F)CC1)c1ccc(CNC(=O)N2CC[C@@H](O)C2)c(F)c1F]

From Intermediate 3 and (R)-pyrrolidin-3-ol, the title compound was obtained using a method similar to the one described in Example 1.425. LCMS m/z=590.4 [M+H]⁺.

### Example 1.433

### Preparation of N-(4-(4,5-dichloro-2-(4-(3,3,3-trifluoropropyl)piperazin-1-yl)phenylcarbamoyl)-2,3-difluorobenzyl)-4-(hydroxymethyl)piperidine-1-carboxamide (Compound 426)

### Step A: Preparation of 1-(4,5-Dichloro-2-nitrophenyl)-4-(3,3,3-trifluoropropyl)piperazine

A mixture of 1,2-dichloro-4-fluoro-5-nitrobenzene (0.73 g, 3.48 mmol), 1-(3,3,3-trifluoropropyl)piperazine (0.633 g, 3.48 mmol) and Et₃N (1.454 mL, 10.43 mmol) in DCM (10 mL) was stirred at room temperature overnight. The solvent was removed under reduced pressure, diluted with EtOAc (30 mL), washed with 1M HCl solution and brine. The organic layer was dried over anhydrous MgSO₄ and concentrated to give the title compound without further purification. LCMS m/z=372.3 [M+H]⁺.

### Step B: Preparation of 4,5-Dichloro-2-(4-(3,3,3-trifluoropropyl)piperazin-1-yl)aniline

To a mixture of 1-(4,5-dichloro-2-nitrophenyl)-4-(3,3,3-trifluoropropyl)piperazine (1.29 g) and nickel(II) chloride hexahydrate (0.826 g, 3.48 mmol) in ethanol (15 mL) was added NaBH₄ (0.658 g, 17.38 mmol) in portions at 0° C. The mixture was stirred at 0° C. for 4 h, and quenched with water. The mixture was extracted with EtOAc (3×30 mL), dried over anhydrous MgSO₄, filtered, and concentrated under reduced pressure to give the title compound (0.75 g) as pale brown solid. LCMS m/z=342.2 [M+H]⁺. ¹H NMR (400 M Hz, CD₃OD) δ ppm 2.85 (m, 2H), 3.16 (m, 4H), 3.52 (m, 6H), 6.90 (s, 1H), 7.08 (s, 1H).

### Step C: Preparation of N-(4-(4,5-Dichloro-2-(4-(3,3,3-trifluoropropyl)piperazin-1-yl)phenylcarbamoyl)-2,3-difluorobenzyl)-4-(hydroxymethyl)piperidine-1-carboxamide

A mixture of 4,5-dichloro-2-(4-(3,3,3-trifluoropropyl)piperazin-1-yl)aniline (50 mg, 0.146 mmol), 4-((tert-butoxycarbonylamino)methyl)-2,3-difluorobenzoic acid (42.0 mg, 0.146 mmol), HATU (83 mg, 0.219 mmol) and TEA (0.061 mL, 0.438 mmol) was taken up in DMF (1 mL) and heated to 50° C. for 18 h in a 5 mL sealed scintillation vial. The mixture was purified by preparative LCMS. To the above purified material was added 0.5 mL of 4 M HCl in 1,4-dioxane solution. The mixture was stirred at room temperature for 4 h, and concentrated under reduced pressure to give the title compound without further purification. LCMS m/z=511.2 [M+H]⁺.

### Step D: Preparation of N-(4-(4,5-Dichloro-2-(4-(3,3,3-trifluoropropyl)piperazin-1-yl)phenylcarbamoyl)-2,3-difluorobenzyl)-4-(hydroxymethyl)piperidine-1-carboxamide (Compound 426)

To a solution of 4-(aminomethyl)-N-(4,5-dichloro-2-(4-(3,3,3-trifluoropropyl)piperazin-1-yl)phenyl)-2,3-difluorobenzamide (30 mg, 0.059 mmol) in DMF (1 mL) was added bis(2,5-dioxopyrrolidin-1-yl) carbonate (15.03 mg, 0.059 mmol). After stirring for 30 min, the reaction was treated with piperidin-4-ylmethanol (6.76 mg, 0.059 mmol). The reaction was heated to 50° C. for 2 h. The crude product was purified by preparative LCMS to give the title compound (6.6 mg) as a white solid. LCMS m/z=652.6 [M+H]⁺.

### Example 1.434

### Preparation of N-(4-(4-Chloro-5-fluoro-2-(4-(3,3,3-trifluoropropyl)piperazin-1-yl)phenylcarbamoyl)-2,3-difluorobenzyl)-4-(hydroxymethyl)piperidine-1-carboxamide (Compound 427)

### Step A: Preparation of 1-(5-Chloro-4-fluoro-2-nitrophenyl)-4-(3,3,3-trifluoropropyl)piperazine

A mixture of 1-chloro-2,5-difluoro-4-nitrobenzene (0.5 g, 2.58 mmol), 1-(3,3,3-trifluoropropyl)piperazine (0.518 g, 2.84 mmol) and TEA (1.080 mL, 7.75 mmol) in DCM (15 mL) was stirred at room temperature overnight. The solvent was removed under reduced pressure. The residue was dissolved into EtOAc, washed with 1M HCl solution and brine. The organic layer was dried over anhydrous MgSO₄, filtered, and concentrated under reduced pressure to give the title compound without further purification. LCMS m/z=356.2 [M+H]⁺.

### Step B: Preparation of 4-Chloro-5-fluoro-2-(4-(3,3,3-trifluoropropyl)piperazin-1-yl)aniline

To a mixture of 1-(5-chloro-4-fluoro-2-nitrophenyl)-4-(3,3,3-trifluoropropyl)piperazine (0.65 g, 1.827 mmol) and nickel(II) chloride hexahydrate (0.521 g, 2.193 mmol) in ethanol (15 mL) was added NaBH₄ (0.346 g, 9.14 mmol) in portions at 0° C. The mixture was stirred at 0° C. for 4 h, and then it was quenched with water, extracted with EtOAc (3×30 mL), and dried over anhydrous MgSO₄. It was filtered and concentrated under reduced pressure. The product was used without further purification. Exact LCMS m/z=326.3 [M+H]⁺. ¹H NMR (400 M Hz, CD₃OD) δ ppm 2.45 (m, 2H), 2.68 (m, 6H), 2.86 (m, 4H), 6.57 (d, T=12 Hz, 1H), 6.96 (d, T=8 Hz, 1H).

### Step C: Preparation of 4-(Aminomethyl)-N-(4-chloro-5-fluoro-2-(4-(3,3,3-trifluoropropyl)piperazin-1-yl)phenyl)-2,3-difluorobenzamide

A mixture of 4-chloro-5-fluoro-2-(4-(3,3,3-trifluoropropyl)piperazin-1-yl)aniline (50 mg, 0.154 mmol), 4-((tert-butoxycarbonylamino)methyl)-2,3-difluorobenzoic acid (44.1 mg, 0.154 mmol), HATU (88 mg, 0.230 mmol) and TEA (64.2 μL, 0.461 mmol) was taken up in DMF (1 mL) and heated to 50° C. for 18 h in a 5 mL sealed scintillation vial. The mixture was purified by preparative LCMS. To the above purified material was added 0.5 mL of HCl in 1,4-dioxane solution. The mixture was stirred at room temperature for 4 h, and concentrated under reduced pressure to give the title compound without further purification. LCMS m/z=495.4 [M+H]⁺.

### Step D: Preparation of N-(4-(4-Chloro-5-fluoro-2-(4-(3,3,3-trifluoropropyl)piperazin-1-yl)phenylcarbamoyl)-2,3-difluorobenzyl)-4-(hydroxymethyl)piperidine-1-carboxamide (Compound 427)

To a solution of 4-(aminomethyl)-N-(4-chloro-5-fluoro-2-(4-(3,3,3-trifluoropropyl)piperazin-1-yl)phenyl)-2,3-difluorobenzamide (25 mg, 0.051 mmol) in DMF (1 mL) was added bis(2,5-dioxopyrrolidin-1-yl) carbonate (12.94 mg, 0.051 mmol). After stirring for 30 min, the reaction was treated with piperidin-4-ylmethanol (5.82 mg, 0.051 mmol). The reaction was heated to 50° C. for 2 h. The mixture was purified by preparative LCMS to give the title compound (2.9 mg) as a white solid. LCMS m/z=636.8 [M+H]⁺.

### Example 1.435

### Preparation of N-(4-Chloro-2-(4-(3,3,3-trifluoropropyl)piperazin-1-yl)phenyl)-4-((2,5-dioxoimidazolidin-1-yl) methyl)-2,3-difluorobenzamide (Compound 428)

Bis(2,5-dioxopyrrolidin-1-yl) carbonate (5.91 mg, 0.023 mmol) was dissolved in DMF (0.1 mL). Then, Intermediate 3 (10 mg, 0.021 mmol) dissolved in DMF (0.1 mL) and added to the above solution. The reaction was stirred at room temperature for 10 min. tert-Butyl 2-aminoacetate (3.3 mg, 0.025 mmol) was added. The reaction was heated to 80° C. and stirred at this temperature for 1 h. HCl (4 M in dioxane) (105 μL, 0.419 mmol) was added to the reaction. The reaction was heated at 80° C. and stirred at this temperature for 2 h. The reaction mixture was purified by preparative LC/MS to give the title compound (8.1 mg). LCMS m/z=560.4 [M+H]⁺.

### Examples 1.436 to 1.439

The following compounds were prepared using the disclosed intermediates and a method similar to the one described in Example 1.435.

| | | | Product |
|---|---|---|---|
| Example | Cmpd | | (Mass Observed |
| No. | No. | Intermediates Used | LC/MS m/z) |
| 1.436 | 429 ¹ | Intermediate 3 and | 574.4 [M + H]⁺ |
| | | tert-butyl 3-amino- | |
| | | propanoate hydro- | |
| | | chloride | |
| 1.437 | 430 ² | Intermediate 3 and | 588.4 [M + H]⁺ |
| | | tert-butyl 4-amino- | |
| | | butanoate hydro- | |
| | | chloride | |
| 1.438 | 431 ³ | Intermediate 3 and | 574.4 [M + H]⁺ |
| | | tert-butyl 2-(methyl- | |
| | | amino)acetate hydro- | |
| | | chloride | |
| 1.439 | 432 | Intermediate 3 and | 616.4 [M + H]⁺ |
| | | (S)-tert-butyl 2- | |
| | | amino-4-methyl- | |
| | | pentanoate hydro- | |
| | | chloride | |

¹ Compound 429: ¹H NMR (400 M Hz, DMSO-d₆) δ ppm 2.66-2.77 (m, 3H), 2.79-2.96 (m, 3H), 2.98-3.25 (m, 5H), 3.24-3.34 (m, 5H), 4.93 (s, 2H), 7.14 (t, J = 7.14 Hz, 1H), 7.28 (dd, J = 8.78, 2.34 Hz, 1H), 7.33-7.40 (m, 1H), 7.57 (t, J = 6.95 Hz, 1H), 7.97 (s, 1H), 8.16 (d, J = 8.59 Hz, 1H), 9.70 (d, J = 4.67 Hz, 1H).
² Compound 430: ¹H NMR (400 M Hz, DMSO-d₆) δ ppm 1.88-2.02 (m, 2H), 2.55-2.61 (m, 2H), 2.90-3.03 (m, 2H), 3.05-3.19 (m, 3H), 3.41-3.53 (m, 2H), 3.55-3.58 (m, 4H), 3.66-3.73 (m, 3H), 4.55 (d, J = 6.06 Hz, 2H), 7.22-7.37 (m, 3H), 7.61 (t, J = 7.20 Hz, 1H), 8.16 (d, J = 8.46 Hz, 1H), 8.89 (t, J = 6.06 Hz, 1H), 9.69 (d, J = 4.67 Hz, 1H), 10.81 (bs, 1H).
³ Compound 431: ¹H NMR (400 M Hz, DMSO-d₆) δ ppm 1.18 (t, J = 7.26 Hz, 1H), 2.75-2.96 (m, 6H), 2.98-3.23 (m, 8H), 4.06 (s, 2H), 4.70 (s, 2H), 7.22-7.31 (m, J = 8.72, 2.27 Hz, 2H), 7.36 (d, J = 2.15 Hz, 1H), 7.60 (t, J = 6.95 Hz, 1H), 8.15 (d, J = 8.21 Hz, 1H), 9.72 (d, J = 4.55 Hz, 1H).

### Example 2

### Preparations of Hydrates, Solvates, Salts, and Free Base of (S)-4-(1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) and Crystalline Forms thereof

Preparations and solid-state analyses are described below for the following salt and/or crystalline forms:
(S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide;
(S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride;
(S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride hydrate;
(S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide dihydrochloride small-channel solvate (MeOH or water);
(S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide sulfate solvate; and
(S)-4-((1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide di-mesylate.

### Example 2.1

### Powder X-Ray Diffraction

Powder X-ray Diffraction (PXRD) data were collected on an X'Pert PRO MPD powder diffractometer (PANalytical, Inc.) with a Cu source set at 45 kV and 40 mA, Cu(Kα) radiation and an X'Celerator detector. Samples were added to the sample holder and smoothed flat with a spatula and weigh paper. With the samples spinning, X-ray diffractogram was obtained by a 12-min scan over the range 5-40 °2θ. Diffraction data were viewed and analyzed with the X'Pert Data Viewer Software, version 1.0a and X'Pert HighScore Software, version 1.0b.

### Example 2.2

### Differential Scanning Calorimetry

Differential scanning calorimetry (DSC) studies were conducted using a TA Instruments, Q2000 at heating rate 10° C./min. The instrument was calibrated for temperature and energy using the melting point and enthalpy of fusion of an indium standard. Thermal events (desolvation, melting, etc.) were evaluated using Universal Analysis 2000 software, version 4.1D, Build 4.1.0.16.

### Example 2.3

### Thermal Gravimetric Analysis

Thermogravimetric analyses (TGA) were conducted using a TA Instruments TGA Q5000 at heating rate 10° C./min. The instrument was calibrated using a standard weight for the balance, and Alumel and Nickel standards for the furnace (Curie point measurements). Thermal events such as weight-loss are calculated using the Universal Analysis 2000 software, version 4.1D, Build 4.1.0.16.

### Example 2.4

### Dynamic Moisture-Sorption (DMS)

A dynamic moisture-sorption (DMS) study was conducted using a dynamic moisture-sorption analyzer, VTI Corporation, SGA-100. Samples were prepared for DMS analysis by placing 5 mg to 20 mg of a sample in a tared sample holder. The sample was placed on the hang-down wire of the VTI balance. A drying step was run, typically at 40° C. and 0.5-1% RH for 1 h. The isotherm temperature is 25° C. Defined % RH holds typically ranged from 10% RH to 90% RH, with intervals of 10 to 20% RH. A % weight change smaller than 0.010% over 10 min, or up to 2 h, whichever occurred first, was required before continuing to the next % RH hold. The water content of the sample equilibrated as described above was determined at each % RH hold.

### Example 2.5

### Preparation of Crystal forms of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170)

### Example 2.5A

Sample was made by adding 2 equivalents of NaOH (using 0.1-N NaOH) to neutralize the dihydrochloride salt. Resulting pH was ˜9.8, added dropwise 0.1 N HCl to reduce pH to a final pH of ˜8.7. Sample was left stirring in a Reacti-Therm™ block. Temperature of Reacti-Therm™ block was 27.4° C. when the sample was removed from stirring. Suction filtration was used to isolate the solid. Solid was then rinsed with water, about 2 times the original volume. The sample was transferred from the filter into a tared vial. The damp sample was dried using gentle heating in a Reacti-Therm™ block and gentle air flow (temperature ˜30° C.) for 1-2 h. Yield was about 91%. This solid was then slurried in ethanol overnight at 27° C. The suspension thickened such that a small amount of ethanol was added to aid transfer of the suspension for centrifuge filtration. Once isolated the solid was allowed to dry open to ambient air for ˜30 min prior to running PXRD.

The powder X-ray diffractogram for the crystalline form of Compound 170 as prepared using the procedure described in Example 2.5A is shown in FIG. 7.

The DSC thermogram for the crystalline form of Compound 170 as prepared using the procedure described in Example 2.5A is shown in FIG. 8.

### Example 2.5B

Compound 170 dihydrochloride (1 g) was weighed into a 20 mL clear glass vial. To the vial was added 10 mL of water and vortexed for 20 second resulting in a clear solution with a pH of 2.91. A solution of 1.0 N NaOH was added (0.50 mL) to give a white solid precipitate. The pH of the suspension was 3.94. Additional amount of 1.0 N NaOH was added until a pH of 12.20 was reached, water was added as needed to thin the suspension to facilitate stirring. At this point, the solid free base was isolated by filtration and washed with DI water.

The solid free base was slurried for 2 weeks in acetone, EtOH, MEK, IPA, MeOH, or ACN at ambient temperature to produce a stable polymorph at ambient temperature.

The powder X-ray diffractogram for the crystalline form of Compound 170 as prepared using the procedure described in Example 2.5B is shown in FIG. 9.

The DSC thermogram for the crystalline form of Compound 170 as prepared using the procedure described in Example 2.5B is shown in FIG. 10.

The dynamic moisture-sorption (DMS) profile for the crystalline form of Compound 170 as prepared using the procedure described in Example 2.5B is shown in FIG. 11. This polymorph of the free base is non-hygroscopic, increasing by only ˜0.1% weight at 90% RH and 25° C.

### Example 2.6

### Preparation of (S)-4-(1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) Dihydrochloride

The crystalline form of Compound 170 dihydrochloride was prepared as described in Example 1.218.

The powder X-ray diffractogram for the crystalline form of Compound 170 dihydrochloride is shown in FIG. 12.

The TGA thermogram for the crystalline form of Compound 170 dihydrochloride is shown in FIG. 13.

The dynamic moisture-sorption (DMS) profile for the crystalline form of Compound 170 dihydrochloride is shown in FIG. 14.

### Example 2.7

### Preparation of (S)-4-(1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) Dihydrochloride Hydrate

Method A

Compound 170 dihydrochloride hydrate was made directly from the free base by salt formation as described below.

Compound 170 (free base) was dissolved in acetone at ˜50 mg/mL. The sample was heated to 50° C. in a Reacti-Therm™ block with stirring and 1 mole equivalent of HCl was added in an attempt to make the mono-HCl salt; percent water by volume was ˜4% after addition of 2N HCl. The sample remained clear. The heat from the Reacti-Therm™ was turned off, allowing the sample to cool to near ambient temperature with stirring; after overnight stirring the temperature reading was 28.4° C. for the Reacti-Therm™ block. The sample remained clear. Drops of MTBE were added until the sample became cloudy. The sample was placed in a 0-5° C. refrigerator. After 0.5 h, the sample was removed from the refrigerator and placed on a stir plate and stirred for 3 days at ambient/stir plate temperature. At this time the temperature on the stir plate was 25.1° C. and the sample was recovered by centrifuge filtration. The solid was allowed to dry in open air for 4 h prior to running PXRD.

Method B

Compound 170 dihydrochloride hydrate form was also made by a slurry of dihydrochloride in solvent systems having a water activity of ≧0.75.

The powder X-ray diffractogram for the dihydrochloride hydrate crystalline form of Compound 170 is shown in PXRD FIG. 15.

The TGA thermogram for the dihydrochloride hydrate crystalline form of Compound 170 is shown in FIG. 16.

The dynamic moisture-sorption (DMS) profile for the dihydrochloride hydrate crystalline form of Compound 170 is shown in FIG. 17.

### Example 2.8

### Preparation of (S)-4-(1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) Dihydrochloride Solvate (MeOH or Water)

Method A

Compound 170 dihydrochloride solvate was made directly from the free base by salt formation as described below.

Compound 170 (free base) was dissolved in acetone at ˜50 mg/mL. The sample was heated to 50° C. in a Reacti-Therm™ block with stirring and 2 mole equivalents of HCl were added; percent water was ˜4% after addition of 4 N HCl. The sample showed immediate precipitation. The heat from the Reacti-Therm™ block was turned off; the sample was allowed to cool to near ambient temperature with overnight stirring. Temperature reading was 28.4° C. in the Reacti-Therm™ block the following day. The sample remained a suspension. Seeds of an existing lot of Compound 170 dihydrochloride were added. No noticeable change to the suspension was observed after the seeds were added. Drops of MTBE were added and the sample was placed in a 0-5° C. refrigerator. After 0.5 h, the sample was removed from the refrigerator and placed on a stir plate to stir for 3 days at ambient temperature. At this time the temperature on the stir plate was 25.1° C. and the sample was recovered by centrifuge filtration. The solid was allowed to dry in open air for 4 h prior to running PXRD.

Method B

Compound 170 dihydrochloride solvate was also made by a slurry of dihydrochloride in solvent systems having a water activity of ≈0.25. A form indistinguishable by PXRD can be formed in solvent systems containing ˜30% methanol or more.

The powder X-ray diffractogram for the dihydrochloride solvate (MeOH or water) crystalline form of Compound 170 is shown in FIG. 18.

The TGA thermogram for the dihydrochloride solvate (MeOH or water) crystalline form of Compound 170 is shown in FIG. 19.

The dynamic moisture-sorption profile for the dihydrochloride solvate (MeOH or water) crystalline form of Compound 170 is shown in FIG. 20.

### Example 2.9

### Preparation of (S)-4-(1-amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) Sulfate Solvate

Compound 170 (free base) was dissolved in acetone at ˜55 mg/mL at room temperature; after complete dissolution, a very small amount of flocculent solid appeared to precipitate. The salt was prepared using 1 mole sulfuric acid per 1 mole of Compound 170. 2.075M sulfuric acid was used and the addition was performed at room temperature producing a sample with ˜4.5% v/v water. A solid precipitated immediately. A stir bar was placed in the vial and the sample was allowed to stir at 40° C. Close observation uncovered that a portion of the product appeared to be a yellow-brown sticky gum or oil phase stuck to the bottom of the vial and a white suspended product. Agitation of the sample was continued for ˜2 h at 40° C. Heat was removed and the resulting mixture was stirred overnight in a Reacti-Therm™ block at 26-27° C. The following day the sample was removed from stirring and allowed to stand for 2 h. The sample was centrifuge filtered and the isolated solid was allowed to dry on the bench for ˜3 h open. The product formed an agglomerate upon drying that was broken up into a powder.

The powder X-ray diffractogram for the sulfate salt solvate crystalline form of Compound 170 is shown in FIG. 21.

The TGA thermogram for the crystalline form of Compound 170 sulfate solvate is shown in FIG. 22.

### Example 2.10

### Preparation of (S)-4-(1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170) Di-Mesylate

Compound 170 (free base) was dissolved in acetone at 46 mg/mL at room temperature. The sample was placed in a Chemglass™ hotplate stirring module at 40° C. To the sample was added 2 mole equivalents of methanesulfonic acid to allow a 1:2 salt (di-mesylate). A precipitation was immediately observed. The sample was stirred at 40° C. for 30 minutes and then moved to a Reacti-Therm™ block at 26° C. to stir overnight. The sample was removed from stirring and centrifuged to isolate the solid (no filter). The supernatant was removed and the product rinsed with neat acetone followed by centrifugation (no filter) to recover the solid. The supernatant was removed and the isolated solid was allowed to dry for about 45 minutes open to ambient air in the Chemglass hotplate set at 40° C. The isolated product was poorly crystalline, but was ripened by exposure to high humidity.

The powder X-ray diffractogram for the crystalline form of Compound 170 di-mesylate is shown in FIG. 23.

The TGA thermogram for the crystalline form of Compound 170 di-mesylate is shown in FIG. 24.

The dynamic moisture-sorption profile for the crystalline form of Compound 170 di-mesylate is shown in FIG. 25.

### Example 3

### Mas Receptor Signaling Assays

### Example 3.1

### Homogeneous Time-Resolved Fluorescence (HTRF®) IP1 Assay

Human and rat Mas receptors were either transiently or stably expressed in HEK293 cells. For transient transfections, human or rat Mas in pHM6 vector were transfected into HEK293 cells using Lipofectamine® (Invitrogen #18324-012). HEK293 cells transfected with empty pHM6 vector were used as a control. For generation of stable cell lines, cDNA expression plasmids encoding human or rat Mas genes and the neo^{r} gene were transfected into HEK293 cells (ATCC# CRL-1573) using Lipofectamine® (Invitrogen) according to the manufacturer's instructions. Stable receptor expressing pools were then generated over 3 weeks by standard techniques in the presence of 500 μg/mL Geneticin® (Gibco). Individual receptor stable pools were dilution cloned using standard techniques and clones were comparatively evaluated in inositol phosphate accumulation assays. Preferred clones were banked and cultured as needed. HEK293 cells were used as controls. The 384 well IP-One HTRF® Assay (Cisbio #62P1APEJ) was performed as described by the manufacturer's protocol. Cells were plated in at 100,000 cells per well in 15 μL DMEM (Gibco #11960) and incubated in a CO₂ incubator at 37° C. for 2 h. Five μL of compounds diluted in 2× stimulation buffer plus 0.4% BSA (Sigma (#A3059) were added to each well and serial diluted IP1 standards (Cisbio #62IP1CDA) were also added to corresponding wells at this step. After the cells were incubated for 4 h in a CO₂ incubator at 37° C., d2-labeled IP1 and cryptate-labeled anti-IP1 monoclonal antibody diluted in lysis buffer were added sequentially in 10 μL per well and the assay plates were kept in the dark at room temperature overnight. Ratiometric measurements of fluorescence emission at 665 nm and 620 nm were obtained using a Pherastar fluorometer (BMG Labtech). IP1 levels in each well were calculated according to the standard curves on each plate. IC₅₀ values were obtained by fitting data to a nonlinear curve-fitting program (GraphPad Software, Inc., La Jolla Calif.).

The observed IC₅₀ values for several compounds of the present invention at the rat Mas receptor are listed in Table B.1.

TABLE B.1
(Rat Mas Receptor)

| Cmpd No. | IC₅₀ (nM) |
|---|---|
| 16 | 315.8 |
| 27 | 650.2 |
| 41 | 581.9 |
| 54 | 497.3 |
| 67 | 82.0 |
| 79 | 133.4 |
| 90 | 704.4 |
| 103 | 535.2 |
| 117 | 372.6 |
| 130 | 252.4 |
| 144 | 108.5 |
| 157 | 183.6 |
| 168 | 27.2 |
| 170 | 21.4 |
| 178 | 44.8 |
| 188 | 29.0 |
| 198 | 173.4 |
| 210 | 68.0 |
| 221 | 157.1 |
| 231 | 50.0 |
| 242 | 315.0 |
| 252 | 114.8 |

Compounds 1 to 481 were tested and observed to have IC₅₀ values in the rat HTRF® IP1 assay ranging from about 100 μM to about 2 nM (Compounds 2, 8, 32, 33, 44, 72, 199, 383, 384, and 442 to 481 were not tested).

The observed IC₅₀ values for several compounds of the present invention at the human Mas receptor are listed in Table B.2.

TABLE B.2
(Human Mas Receptor)

| Cmpd No. | IC₅₀ (nM) |
|---|---|
| 13 | 88.2 |
| 17 | 386.2 |
| 20 | 610.1 |
| 26 | 87.3 |
| 56 | 56.1 |
| 76 | 68.2 |
| 167 | 35.3 |
| 170 | 15.6 |
| 178 | 34.4 |
| 186 | 49.1 |
| 200 | 41.7 |
| 202 | 92.1 |
| 226 | 93.9 |
| 231 | 74.3 |
| 235 | 39.1 |
| 250 | 5.7 |
| 259 | 107.8 |
| 284 | 57.2 |
| 293 | 103.0 |
| 330 | 11.7 |
| 392 | 6.13 |
| 428 | 73.3 |

A total of 140 compounds were tested and observed to have IC₅₀ values in the human HTRF® IP1 assay ranging from about 100 μM to about 3 nM (Compounds 8, 10, 14 to 24, 27 to 55, 57 to 75, 77 to 166, 172 to 177, 179 to 183, 190 to 199, 205 to 206, 208 to 225, 228 to 230, 233, 236 to 248, 251 to 255, 261 to 263, 265, 267 to 269, 274 to 276, 278, 279, 285, 289, 302, 304, 305, 309, 321, 323 to 325, 327, 328, 331 to 350, 353, 355 to 380, 383, 384, 386, 387, 389, 390, 398, 402 to 405, 411, 417, 418, 422, 423, 433, 434, and 441 to 481 were not tested).

### Example 3.2

### Homogeneous Time-Resolved Fluorescence IP-One HTRF® Assay (Cisbio)

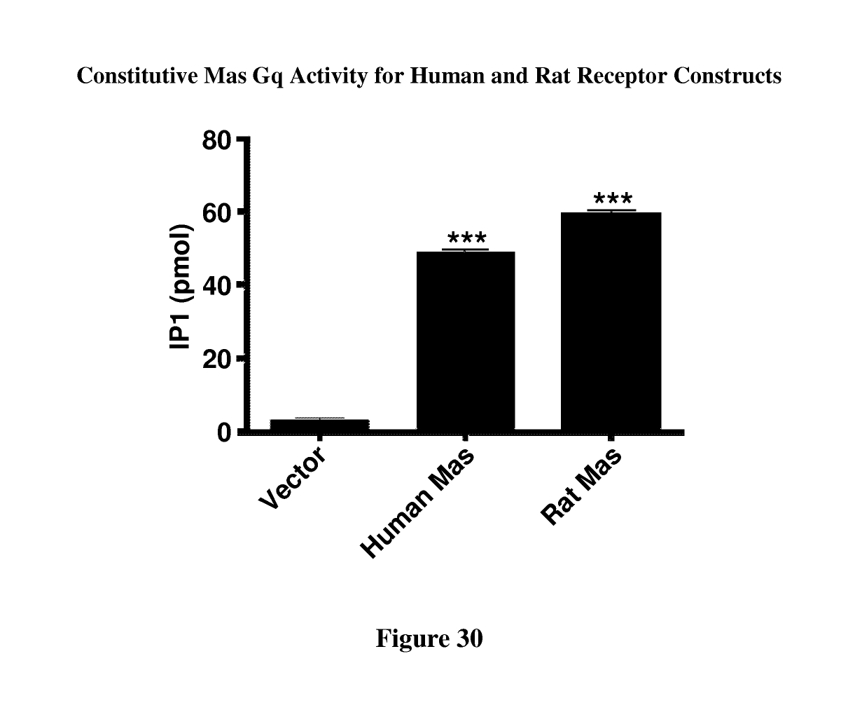

Many GPCRs are able to couple constitutively to their preferred G protein in the absence of ligand. To determine whether the Mas receptor has constitutive G protein coupling activity, human or rat Mas in HEK293 cells were expressed by transient transfection. An antibody to the hemagglutinin (HA) epitope tag on the Mas expression constructs was used to confirm expression by flow cytometry 48 h post transfection (data not shown). G_{q} coupling in these cells was measured by HTRF® IP-one assays. Expression of either human Mas or rat Mas receptor in HEK293 cells resulted in a significant increase in IP1 accumulation compared to cells transfected with empty vector (FIG. 30), indicating constitutive G_{q} coupling of the receptor. Similar results were seen when dog and pig Mas orthologs were transfected into HEK293 cells.

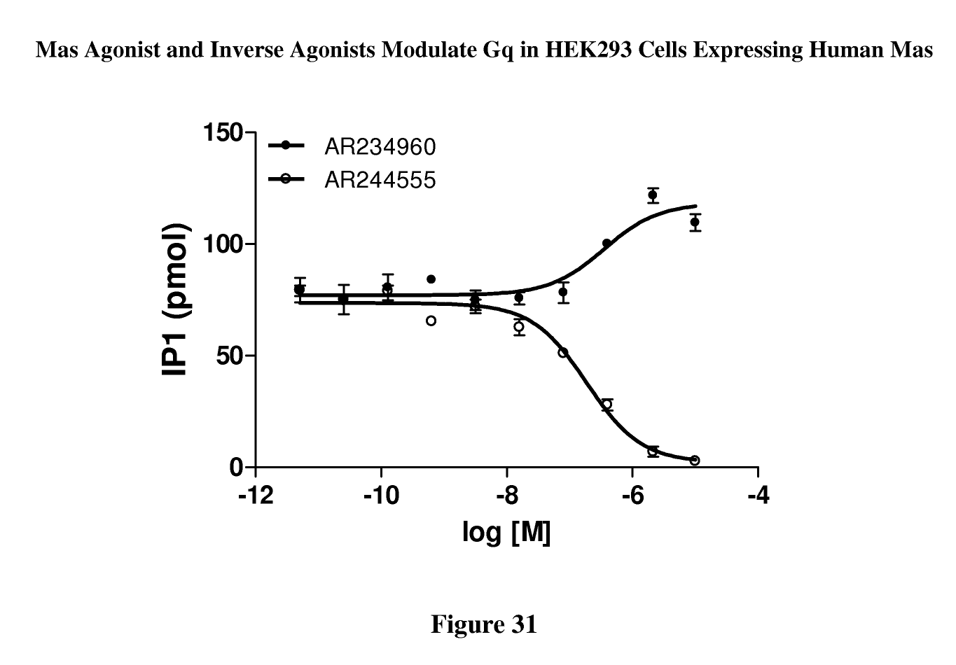

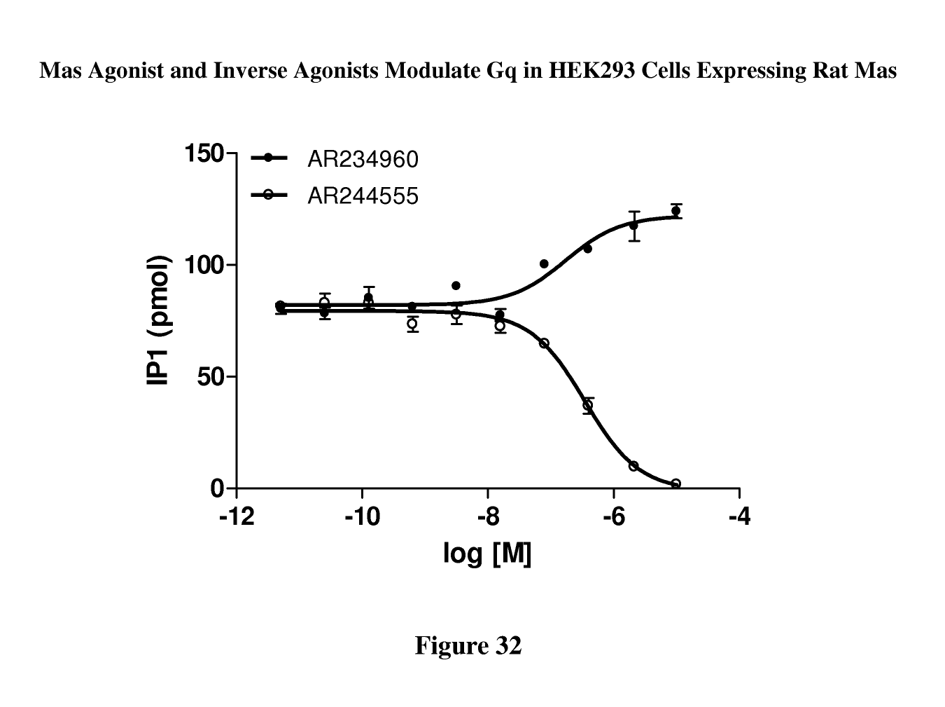

The constitutive G_{q} coupling of the Mas receptor provided a suitable assay signal with which to screen small molecule libraries for Mas receptor modulators. This assay was able to identify and optimize both agonists and inverse agonists to the Mas receptor. Functional G_{q} agonism and inverse agonism for representative compounds (agonist AR234960 and inverse agonist AR244555) was demonstrated in HEK cells stably expressing either human or rat Mas receptor (FIG. 31 and FIG. 32, and TABLE C). There were detected effects of these compounds in control HEK293 cells.

TABLE C
Summary of IP Assay IC₅₀ Data

| Ligand | Human IC₅₀ (μM) | Rat IC₅₀ (μM) |
|---|---|---|
| AR234960 | 0.351 ± 0.055 | 0.172 ± 0.009 |
| | (agonist) | (agonist) |
| AR244555 | 0.186 ± 0.011 | 0.348 ± 0.067 |
| | (inverse agonist) | (inverse agonist) |

Values are means ± SEM

### Example 3.3

### cAMP Assay

cAMP accumulation in HEK293 cells stably expressing human or rat Mas receptors was determined by the 384 well cAMP Dynamic2 Homogenous Time-Resolved Fluorescence (HTRF®) assay (CisBio Cat#62AM4PEB) following the manufacturer's protocol. Briefly, cells were plated at 30,000 or 1000 cells per well in 5 μL stimulation buffer (PBS containing 500 μM IBMX and 0.1% bovine serum albumin), 5 μL of Mas compounds diluted in PBS were added to each well and serial diluted cAMP standards were also added to corresponding wells at this step. For detecting G₁-coupled activities, 10 μM forskolin was included with the compounds for a final concentration of 5 μM at the stimulation step. Following 1 h stimulation at room temperature, d2-labeled cAMP and anti-cAMP cryptate conjugate diluted in detection buffer (included in the kit) were added to cells sequentially at 5 μl per well. The plates were incubated further for 1 h at room temperature. Ratiometic measurements of fluorescence emission at 665 nm and 620 nm were obtained by Pherastar fluorometer (BMG Labtech) and cAMP levels in each well were calculated according to the standard curves on each plate. IC₅₀ values were obtained by fitting data to a nonlinear curve-fitting program (GraphPad Software, Inc., La Jolla Calif.).

Results:

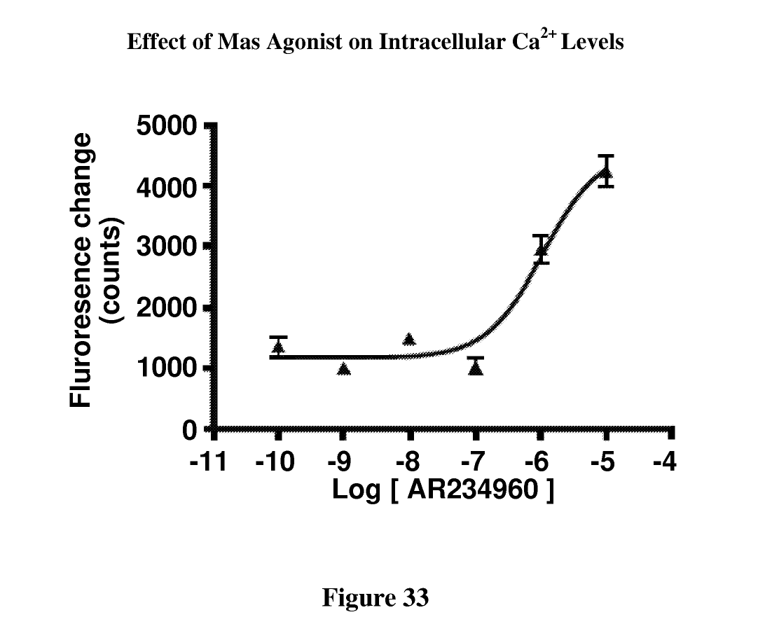

There was no detectable constitutive coupling to Gₛ or Gᵢ in cells stably expressing human or rat Mas receptor. Furthermore, activation of the Gₛ/cAMP pathway by any of the other purported Mas receptor agonists was not detected. However, although no constitutive Mas-Gᵢ signaling was evident in our cAMP assays, the Mas agonist AR234960 was able to stimulate Mas-Gᵢ activity resulting in a dose dependent reduction in forskolin stimulated cAMP levels (FIG. 33).

TABLE D
Summary of cAMP Assay IC₅₀ Data

| | Gᵢ/cAMP IC₅₀ (μM) | | Gₛ/cAMP IC₅₀ (μM) | |
|---|---|---|---|---|
| Ligand | Human | Rat | Human | Rat |
| AR234960 | 0.719 ± 0.012 | 1.710 ± 0.011 | NR | NR |
| | (agonist) | (agonist) | | |
| AR244555 | NR | NR | NR | NR |

Values are means ± SEM; NR = No Response

### Example 3.4

### Ca²⁺ Measurements by Fluorometric Imaging Plate Reader (FLIPR) Assay

HEK293 cells stably expressing human Mas receptors were monitored for changes in intracellular Ca²⁺ using a FLIPR-384 (Molecular Devices, Sunnyvale, Calif.). Cells were seeded into black-walled clear-base 384-well plates at a density of 2×10⁴ cells per well and incubated with Hank's Balanced Salt Solution (HBSS) containing 20 mM HEPES pH 7.4, 2 μM Calcium 3 dye (Molecular Devices Corporation, Sunnyvale, Calif.) and 2.5 mM probenecid at 37° C. for 60 min. Cells were washed with HBSS containing 20 mM HEPES, pH 7.4 and 2.5 mM probenecid and the plates were then placed into the FLIPR instrument to monitor cell fluorescence before and after the addition of the agonists at different concentrations.

Results:

Since GPCR activation of the G_{q}-PLC pathway typically results in increased intracellular calcium, Ca²⁺ was measured in HEK293 cells stably expressing the human Mas receptor. Consistent with its effect on inositol phosphate accumulation, AR234960 elicited a significant increase in intracellular Ca²⁺ in a dose-dependent manner further verifying that Mas activates the downstream G_{q}-PLC-Ca²⁺ pathway.

### Example 4

### Effect of Compounds on Myocardial Ischemia/Reperfusion (UR) Injury

Animals:

Male Sprague-Dawley rats (220-260 g) (Charles River) were housed four per cage and maintained in a humidity-controlled (40-60%) and temperature-controlled (68-72° F.) facility on a 12 h:12 h light/dark cycle with free access to food and water.

Induction of Myocardial I/R Injury:

Adult rats were anesthetized with sodium pentobarbital (50 mg/kg i.p.) and placed in the supine position on a surgery frame with a heating pad (37° C.) beneath. Rats were tracheostomized and ventilated with a SAR-830 small-animal ventilator (Model 683, Harvard Apparatus) to provide room air at a tidal volume of 2.5 mL/stroke and at a rate of 70 stroke/min. Polyethylene catheters were placed in the right internal carotid artery and the external jugular vein for measurement of mean arterial blood pressure and infusion of drug or vehicle, respectively.

Myocardial I/R injury was produced as follows. A left thoracotomy was performed approximately 20 mm from the sternum to expose the heart at the fifth intercostal space. The pericardium was removed, and the left atrial appendage was moved to reveal the location of the left coronary artery. A ligature (6-0 prolene), along with a snare occluder, was placed around the left coronary artery close to the place of origin. After surgical preparation, the rat was allowed to stabilize for 15 min. Regional myocardial ischemia was produced by tightening the previously placed reversible ligature around the coronary artery to completely occlude the vessel. Sham-operated animals underwent the same surgical procedures but without ligation of the coronary artery. The ligature was untied after 30 min, and the ischemic myocardium was reperfused for 2 h.

Animals were randomly divided into the following 4 groups: (1-3) low, medium, and high doses of Compound 170 administered by continuous i.v. infusion starting 30 min before coronary artery ligation (n=6 per dose), and (4) 20% HPBCD (vehicle, hydroxypropyl-β-cyclodextrin) administered by continuous i.v. infusion beginning 30 min before coronary artery ligation (n=6). All the rat hearts listed above were used for calculation of myocardial infarct size (see below). Mean arterial blood pressure was measured after stabilization and just prior to drug infusion (baseline) and again following 25 min of drug or vehicle infusion, before coronary artery ligation.

Measurement of Infarct Size:

After ischemia and reperfusion treatment, the left coronary artery was re-occluded, and 5% Evans blue dye (1 mL) was administered to the circulation via the jugular cannula and allowed to perfuse the non-ischemic portions of the heart. The myocardium not stained with Evans blue dye represents the ischemia area at risk (AAR). Within the area at risk, ischemic injury (infarct) was measured by TTC staining and expressed as myocardial infarct size. Briefly, the entire heart was excised, rinsed of excess Evans blue dye, trimmed of atrial tissue, and sliced transversely into sections 2 mm thick. These slices were incubated in a 1% solution of TTC for 12 minutes to stain the viable myocardium brick red. The samples were then fixed in a 10% formalin solution for 24 h and both sides of each slice were photographed with an Olympus OM2 camera using a 90-mm macrolens and a 2× teleconverter. The ischemic risk area (unstained by Evans blue dye) and the infarcted area (unstained by TTC) were outlined on each photograph and measured by planimetry. The area from each region was averaged from the photographs of each side for each slice. Infarct size was expressed as a percentage of the ischemic area at risk.

Drug Treatment:

Rats were dosed i.v. via the jugular cannula with vehicle or test compound in a single bolus administration (loading dose) followed immediately by continuous i.v. infusion using a Harvard Apparatus 11+ syringe pump at a flow rate of 1 mL/kg/h. Compound 170 was dosed at, 0.626 mg/kg (loading)+0.164 mg/kg/h (low dose), 1.878 mg/kg (loading)+0.492 mg/kg/h (medium dose), and 6.26 mg/kg (loading)+1.64 mg/kg/h (high dose).

Results:

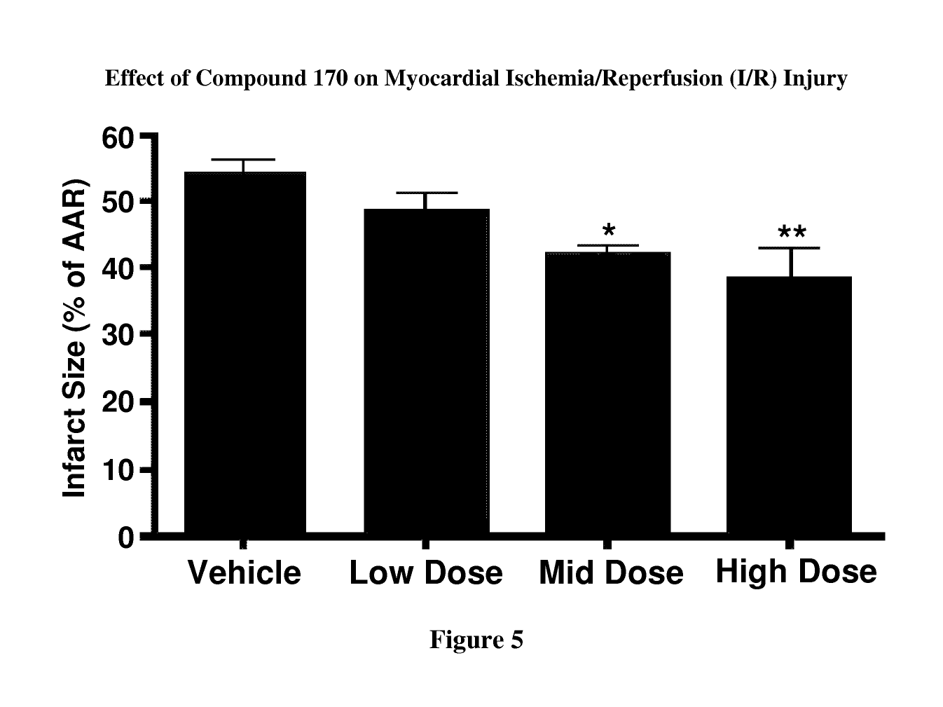

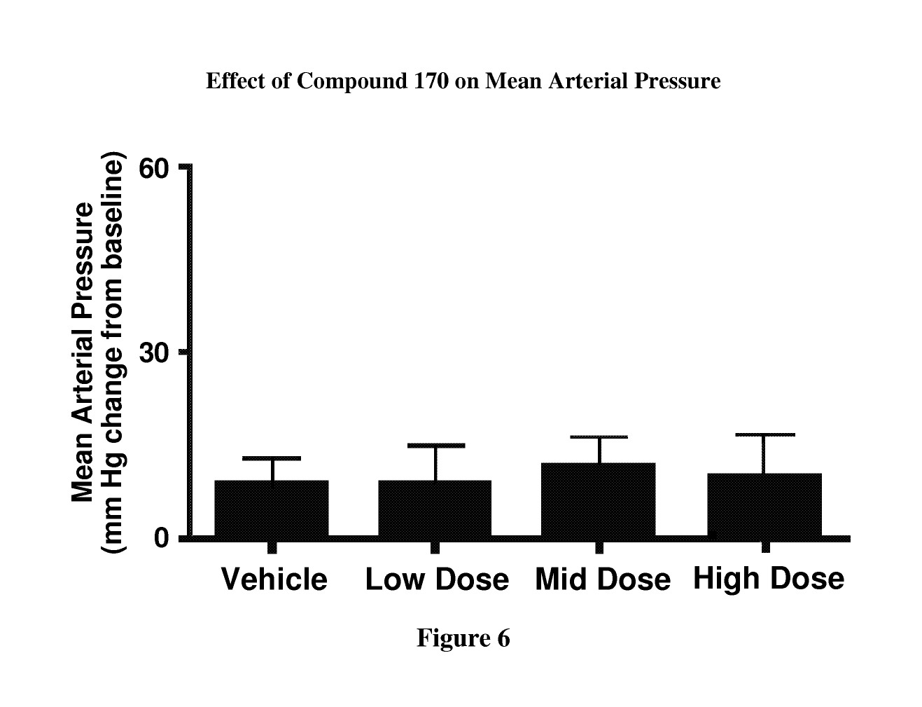

An example of a compound of the invention tested in this assay is shown in FIG. 5. In this example, Compound 170 at both the medium and high dose was found to provide protection against ischemia-reperfusion injury in rat hearts as shown by a significant decrease in myocardial infarct size after reperfusion compared to vehicle treatment. In addition, as shown in FIG. 6, Compound 170, at the three doses tested (i.e., low, medium and high), had no significant effect on mean arterial blood pressure (MAP) compared to vehicle treatment.

### Example 5

### Inhibition of Mas G-Protein Signaling Improves Coronary Flow, Reduces Myocardial Infarct Size and Provides Cardioprotection—Mas Expression in the Heart

### Example 5.1

### Cloning of Human and Rat Mas Genes

The cDNA for human and rat Mas genes were obtained by PCR using genomic DNA as templates. The following were used as primer sets:

(human Mas sense primer)
5′-TGGATGGGTCAAACGTGACATCATT-3′;
(human Mas antisense primer)
5′-CGCGGATCCTCAGACGACAGTCTCAACTGTGACC-3′;
(rat Mas sense primer)
5′-ACCAAGCTTGGACCAATCAAATATGACATCCTTTG-3′;
and
(rat Mas antisense primer)
5′-CAAGAATTCAGACCACAGTCTCAATGGATACA-3′.

PCR was performed using Pfu polymerase (Stratagene, San Diego, Calif.) with the buffer system provided by the manufacturer plus 10% DMSO, 2.5 μM of each primer, and 300 μM each of the four nucleotides. After an initial denaturation at 95° C. for 4 min, 30 cycles of 95° C. for 40 s, 60° C. for 50 s, 72° C. for 1 min 40 s were performed, which was followed by a final extension at 72° C. for 7 min. The 986 bp human Mas PCR fragment was digested with BamHI, and cloned into blunted HindIII (5′)-BamHI (3′) sites of expression vector pHM6 (Invitrogen, Carlsbad, Calif.), while the 988 bp rat Mas PCR fragment was cloned into HindIII (5′)-EcoRI (3′) sites of pHM6 after digestion with HindIII and EcoRI.

### Example 5.2

### Chemicals

Mas agonist (AR234960, 14(4-(3-fluorophenyl)-1-(2-methoxy-4-nitrophenylsulfonyl)pyrrolidin-3-yl)methyl)-4-(pyridin-2-yl)piperazine) and inverse agonist (AR244555, (1′-(but-3-enyl)-1,2-dihydro-5-chloro-1-(2,6-difluoro-benzoyl)-spiro[3H-indole-3,4′-piperidine], see WO2005/063745A2, Compound 359) were dissolved in dimethyl sulfoxide (DMSO) for in vitro and ex vivo assays and in 20% hydroxypropyl-β-cyclodextrin (HPBCD) for in vivo experiments.

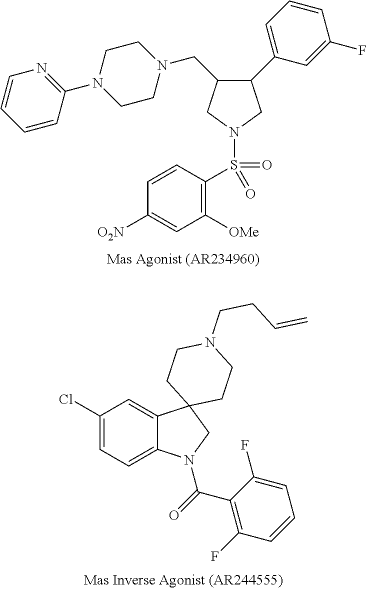 [SMILES: COc1cc([N+](=O)[O-])ccc1S(=O)(=O)N1CC(CN2CCN(c3ccccn3)CC2)C(c2cccc(F)c2)C1 ; C=CCCN1CCC2(CC1)CN(C(=O)c1c(F)cccc1F)c1ccc(Cl)cc12]

PLC inhibitor U-73122 (1-(6-((8R,9S,13S,14S,17S)-3-methoxy-13-methyl-7,8,9,11,12,13,14,15,16,17-decahydro-6H-cyclopenta[a]phenanthren-17-ylamino)hexyl)-1H-pyrrole-2,5-dione) was dissolved in DMSO.

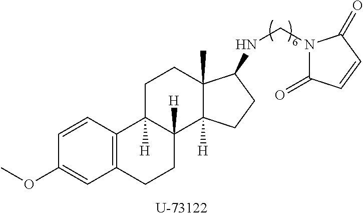

### Example 5.3

### Animals

Male Sprague-Dawley rats (270-330 g) were purchased from Harlan. The Mas knockout mouse line was purchased from Deltagen (San Mateo, Calif.) and confirmation of Mas mRNA deletion was performed by RT-PCR using Mas gene specific primers (sense: TCCCTTGCTGAAGAGAAAGC; anti-sense: ATCTTTGAAAGCCCTGGTCA). All animals were housed in standard cages and were maintained at 25±1° C. under 12-h light and dark cycles. The animals were fed standard diet and water ad libitum.

### Example 5.4

### Preparation of Adenoviral Constructs and Adenoviral Infection of Cultured Cardiomyocytes

Adenoviral constructs were prepared from expression plasmids encoding β-galactosidase (AdLacZ, as a control) or wild-type human Mas receptor (AdMas). Homologously recombinant adenoviruses were generated by Qbiogene (Carlsbad, Calif.). Neonatal rat ventricular myocytes (NRVMs) were purchased from Cell Applications, Inc. (San Diego, Calif.) and plated overnight in serum-containing media at a density of 0.3×10⁶ cells per well in 24-well plates for inositol phosphate assays, or at a density of 0.25×10⁶ cells per well in 2-well chamber slides for immunocytochemistry. After overnight culture, the cells were washed and the medium was replaced with serum-free medium supplemented with insulin/transferrin/selenium (ITS, Sigma). Cells were infected for 6 h with AdLacZ or AdMas adenoviruses (1000 viral particles/cell). Using the control adenovirus encoding “LacZ” (AdLacZ) and β-galactosidase staining of AdLacZ-infected myocytes, it was determined that a viral titer of 1000 viral particles per cell resulted in nearly 100% infection efficiency without cytotoxicity. Cells were subsequently washed and maintained in serum-free medium with supplements for inositol phosphate assay or immunocytochemical staining.

### Example 5.5

### Mas Expression Analysis in Rat and Human Heart

### Example 5.5A

### Immunocytochemistry in Neonatal Rat Ventricular Myocytes (NRVMs)

NRVMs were plated on 2-well chamber slides and infected with adenoviruses as described above. Six h after adenovirus infection, cells were washed and then incubated with either vehicle or the Mas inverse agonist AR244555 at 10 μM for another 42 h. Cells were then fixed with 3.7% formaldehyde, washed with PBS, permeabilized with 0.3% Triton X-100 in PBS and blocked with 10% normal goat serum in PBS. Myocyte sarcomeres (F-actin) were stained with Rhodamin-Phalloidin (Molecular Probes) and visualized on a Zeiss fluorescence microscope. Cell size was quantitated by digital planimetry using Adobe Photoshop.

### Example 5.5B

### RNA Isolation and Semi-Quantitative Reverse Transcription (RT)-PCR

Total RNA was prepared from rat atrial and ventricular tissues using TRIzol® reagent (Invitrogen). First-strand cDNA synthesis was performed using the SuperScript III First-Strand Synthesis System (Invitrogen) according to manufacturer's instructions. Semi-quantitative RT-PCR for the expression of rat Mas (and glyceraldehyde-3-phosphate dehydrogenase (GAPDH) as a control) was performed using Platinum® PCR SuperMix (Invitrogen). Primers used for rat Mas are as follows:

sense:
GTCGGGCGGTCATCATCTTCATA;
and
anti-sense:
ACTCCCCCTGCGGTCCTCA.

Semi-quantitative RT-PCR for mRNA expression of human Mas receptor was performed in a human cardiovascular cDNA panel (AMS Biotechnology) using actin as a control. Human Mas primer sequences were:

sense:
ACGGGCCTCTATCTGCTGACG;
and
anti-sense:
AAGGGTTGGCGCTACTGTTGATT.

### Example 5.5C

### Immunohistochemistry

Snap-frozen heart tissues from male Sprague-Dawley rats were cryosectioned at a thickness of 8 μm and stored at −80° C. Sections were removed from the freezer and allowed to come to room temperature. Sections were fixed with cold acetone, washed with PBS, and blocked with 10% normal goat serum in PBS containing 0.2% Tween (PBST). Sections were incubated with the primary rabbit Mas antibody (Novus Biologicals) diluted 1:100 in PBST containing 1% BSA for overnight at 4° C. Half of the primary antibody solution was pre-absorbed for 30 min at room temperature with 10 μg/mL (final) Mas blocking peptide (Novus Biologicals) and applied to control sections which were incubated overnight at 4° C. Sections were washed three times with PBST and then incubated at room temperature for 45 min with secondary antibody solution composed of Texas Red goat anti-rabbit antibody diluted 1:100 in PBST containing 1% BSA. Sections were then washed with PBS and mounted on glass slides using antifade reagent supplemented with 4′,6-diamidino-2-phenylindole (DAPI; Invitrogen). For co-staining experiments, sections were concurrently stained with Mas antibody and an FITC-conjugated α-smooth muscle actin antibody (Sigma) or a mouse anti-Von Willbrand Factor (VWF) antibody (LifeSpan BioSciences). Immunofluorescence analysis was performed using a Zeiss fluorescence microscope.

Human left ventricular heart sections (AMS Biotechnology) were also used for immunohistochemical detection using a DAB substrate kit (Abcam). Briefly, sections were fixed with acetone, washed with PBS, and blocked with 10% normal goat serum in PBST. Sections were incubated with the primary Mas antibody diluted 1:100 in PBST containing 1% BSA or primary Mas antibody solution pre-absorbed with blocking peptide. Sections were incubated overnight at 4° C., and then washed three times with PBST. Sections were incubated for 15 min in 0.3% H₂O₂/PBS and incubated at room temperature for 45 min with secondary antibody solution composed of HRP-conjugated goat anti-rabbit antibody diluted 1:1000 in PBST containing 1% BSA. After being washed with PBS, sections were stained with DAB Chromagen (Abcam) for 10 min and counterstained with hematoxylin for 1 min Sections were then washed with PBS and dehydrated with ethanol before mounting media and a cover slip were applied. Immunofluorescence analysis was performed on a Zeiss fluorescence microscope.

Results:

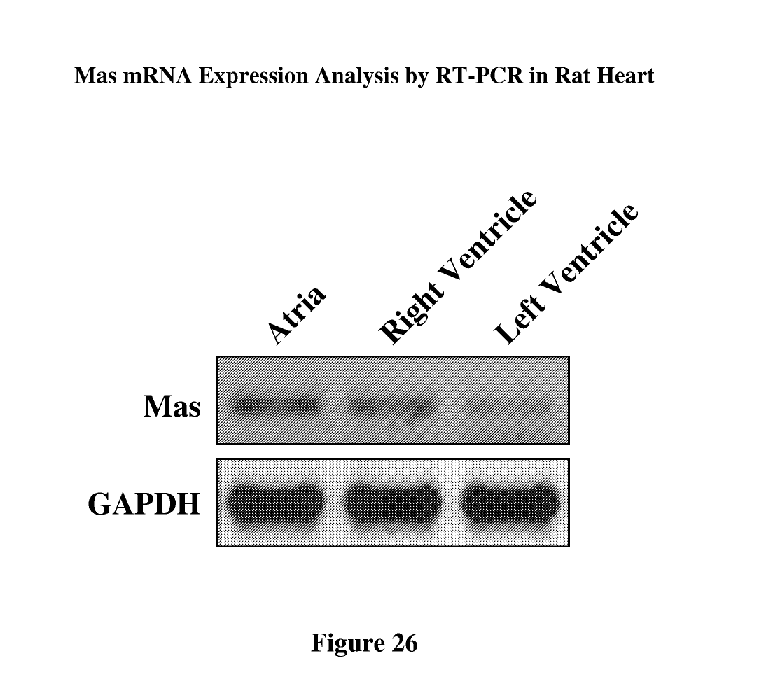

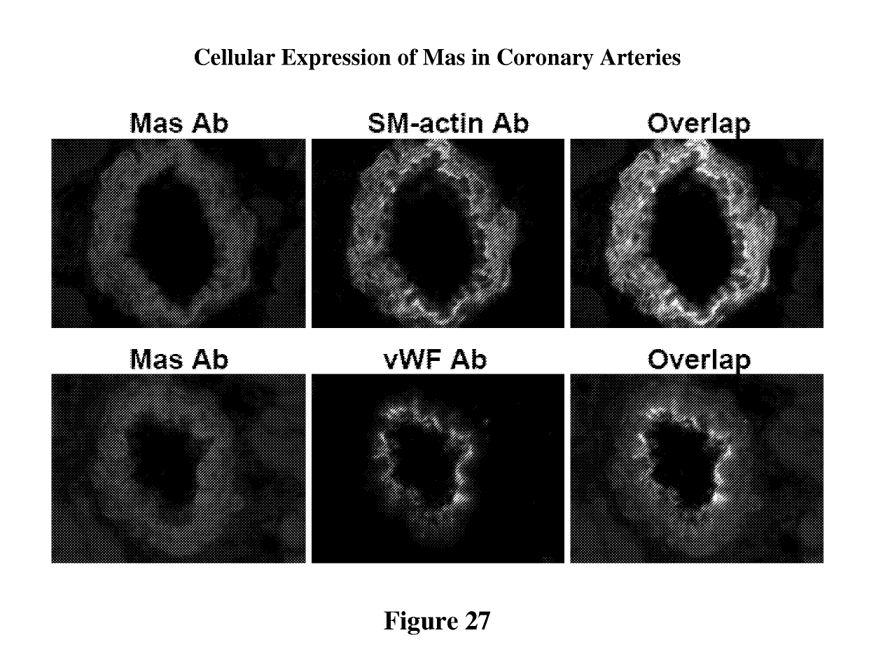

Mas mRNA and protein have been reported to be expressed in rat heart and cardiomyocytes (Tallant et. al., Am. J. Physiol. Heart Circ. Physiol. 289: H1560-H1566 (2005)). To confirm this, RT-PCR and immunohistochemical staining experiments were performed. RT-PCR in rat heart revealed mRNA expression in all chambers (FIG. 26). Expression of Mas protein was evaluated in rat left ventricle by immunohistochemical staining. Mas protein expression was detected in cardiomyocytes and coronary arteries of rat heart. As a control to confirm that the staining was specific for Mas, the antibody solution was pre-absorbed with an equal molar concentration of the corresponding immunogenic peptide prior to incubating with heart sections. The amount of staining was substantially reduced with pre-absorption, verifying that the antibody was specifically recognizing the correct Mas epitope. To clarify which cell types were expressing Mas protein in coronary arteries, left ventricle sections were co-stained concurrently with antibodies for Mas and α-smooth muscle actin (a marker for smooth muscle cells) or with antibodies for Mas and von Willibrand Factor (a marker for endothelial cells). Mas protein expression overlapped with the markers for both smooth muscle cells and endothelial cells, indicating that Mas was expressed in both cell types (FIG. 27).

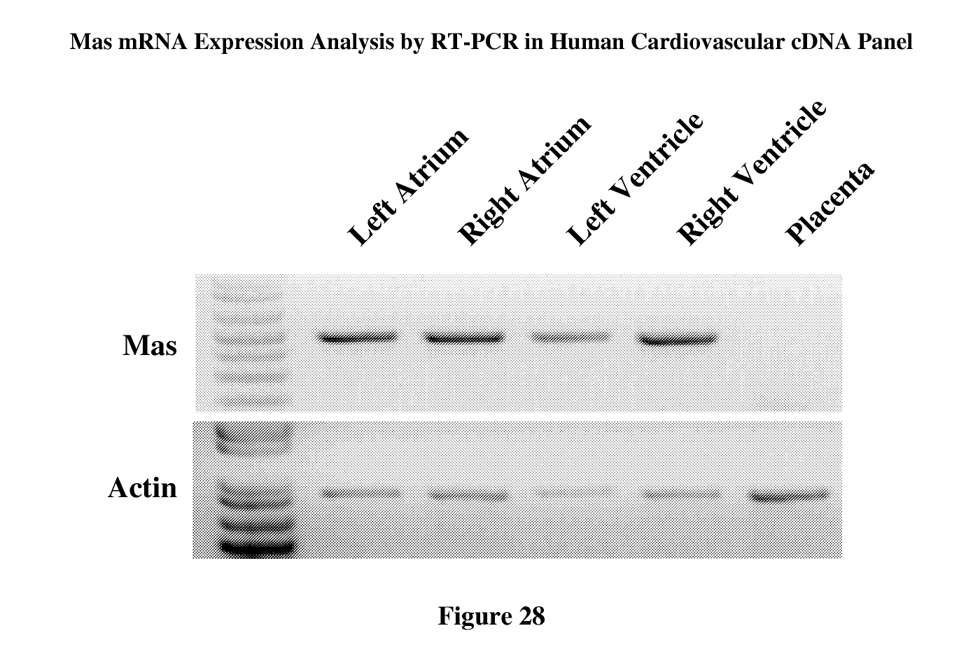

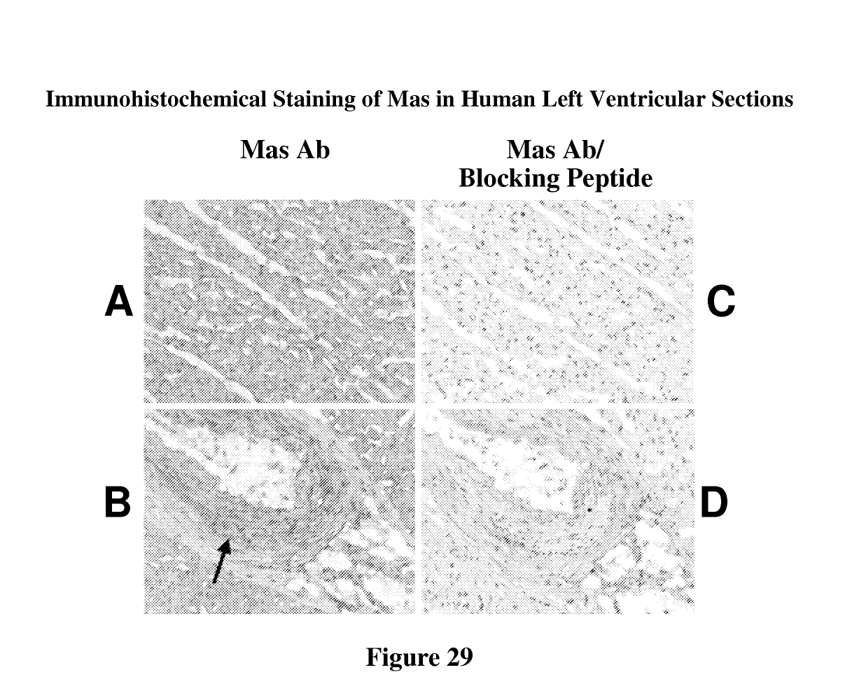

The expression pattern of the Mas receptor in human heart was also examined. RT-PCR analysis with human Mas receptor specific primers in a human cardiovascular cDNA panel demonstrated that the Mas mRNA transcript was detected in all four chambers of human heart, whereas it was not detected in the placenta using the same primers (FIG. 28). Immunohistochemical staining in human left ventricular sections using a Mas specific antibody revealed Mas protein expression in both cardiomyocytes (FIG. 29, Panel A) and coronary arteries (FIG. 29, Panel B). The specificity of Mas antibody was verified in control experiments in which staining was reduced by preincubation of the antibody with the blocking peptide prior to incubation with tissue sections (FIG. 29, Panels C and D).

### Example 5.6

### Decreased Infarct Size in Mas^{-/-} Mice after Ischemia/Reperfusion Injury

### Example 5.6A

### Coronary Artery Ligation Model

Occlusion and reperfusion of the coronary artery was performed in male Mas knockout (Mas^{-/-}) mice and wild type (Mas^{+/+}) controls or in male Sprague-Dawley rats as previously reported (Means et. al., Am. J. Physiol. Heart Circ. Physiol. 292: H2944-H2951 (2007)). Briefly, mice or rats were anesthetized with an intraperitoneal injection of pentobarbital (70 mg/kg) and placed in a supine position under body-temperature control. Each animal was endotracheally intubated and ventilated with a tidal volume of 0.8 mL at a rate of 120 strokes/min (mice) or 2.5 mL at a rate of 70 strokes/min (rats) by using a rodent respirator (Harvard Apparatus). After left thoracotomy, a 8-0 (mice) or 7-0 (rats) surgical suture was passed underneath the left anterior descending coronary artery (LAD) at a position 2 mm from the tip of the left auricle using the aid of a stereoscope (Nikon). PE-10 tubing (1-2 mm in length) was placed along the vessel as a cushion and was secured around the tubing to occlude the LAD. For the sham-operated control animals, the procedure was performed as above except that the suture was not secured around the LAD to occlude the vessel. Myocardial ischemia was verified by blanching of the left ventricle (LV) and by change in electrocardiogram. To induce ischemia/reperfusion injury and determine infarction size the LAD was occluded for 30 min and then the heart was reperfused for 2 h. In experiments using rats with Mas inverse agonist, vehicle (20% HPBCD) or Mas inverse agonist (10 mg/kg) were injected as a bolus through the jugular vein either 10 min before ischemia or 3 min before reperfusion.

### Example 5.6B

### Assessment of LV Area at Risk and Infarct Size

For acute studies, following 2 h of reperfusion, the LAD was reoccluded and 5% Evans blue dye was injected into the LV cavity with a 27-gauge needle to define the nonischemic zone (blue area). The heart was excised immediately and rinsed in saline to remove excess dye, and the LV was cut transversely into five slices of equal thickness. These samples were incubated in 1% 2,3,5-triphenyltetrazolium chloride (TTC)-containing tris-HCl buffer (pH 7.8) at 37° C. for 2×10 min to stain the viable myocardium (red area). The unstained (white) area inside the red area defined the infarcted area. The area at risk (AAR, i.e. the ischemic area) was defined as the white infarcted necrotic tissue plus the red viable salvaged tissue. Each slice was photographed from both sides using a microscope equipped with a high-resolution digital camera. The AAR, infarcted area, and the total LV area were measured by digital planimetry using Adobe Photoshop. Infarct size was expressed as percentage of area at risk. For long-term studies, after measurements of cardiovascular hemodynamics (see below) LV was cut transversely into five slices of equal thickness and the sections were stained with the TTC solution. Infarct size was expressed as a ratio of the infarcted area over the total LV area.

Results: Decreased Infarct Size in Mas^{-/-} Mice after Ischemia/Reperfusion Injury:

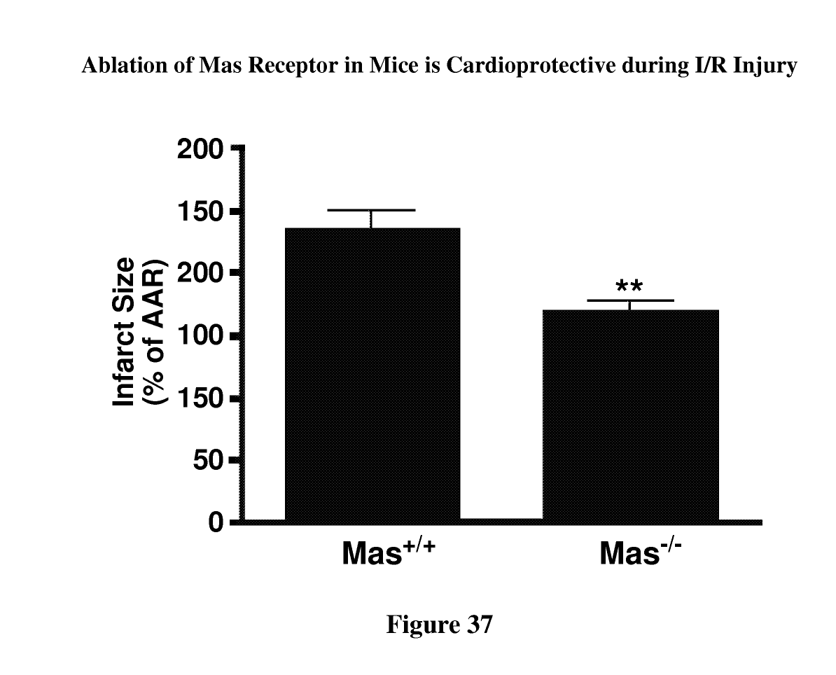

To determine whether the Mas receptor activation might contribute to ischemia/reperfusion injury in vivo, Mas^{+/+} with Mas^{-/-} mice were compared using a well established model of regional myocardial ischemia/reperfusion injury. Myocardial infarction size was measured in hearts exposed to 30 min of left anterior descending coronary occlusion followed by 2 h of reperfusion. The infarct size, expressed as a percentage of the area at risk, was significantly decreased in Mas^{-/-} mice (34%) compared to Mas^{+/+} mice (47%) (FIG. 37). Thus, genetic ablation of the Mas receptor provides protection against in vivo myocardial ischemia/reperfusion injury in the mouse.

Reduction of Myocardial Ischemia/Reperfusion Injury by Pharmacological Inhibition of Mas:

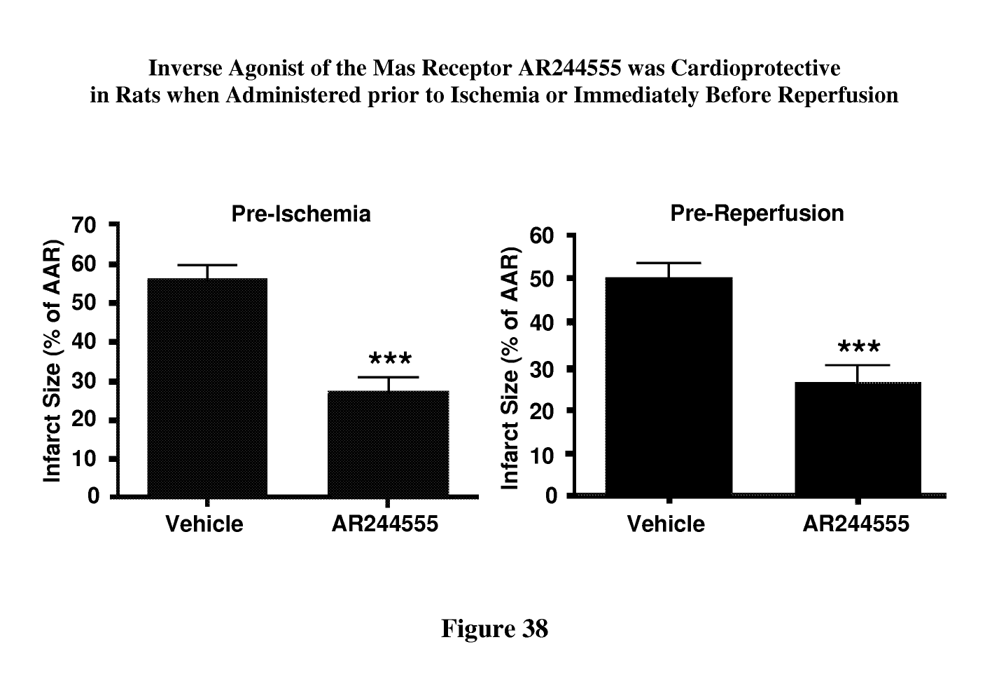

A pharmacological approach was used to verify the role of the Mas-G_{q} signaling pathway in ischemia/reperfusion injury. Since reduced Mas activity in the Mas^{-/-} mice resulted in smaller infarcts, the Mas inverse agonist AR244555 was tested in an in vivo rat ischemia/reperfusion injury model. In these studies, AR244555 was evaluated using two protocols; 1) i.v. bolus administration, then 30 min of ischemia followed by 2 h of reperfusion; and 2) 30 min ischemia, then i.v. bolus administration for 3 min followed by 2 h of reperfusion. Mas inverse agonist AR244555 treatment reduced infarct size by about half when compared with vehicle treatment in both protocols (FIG. 38). These results demonstrate that Mas inverse agonist treatment provides protection from ischemia/reperfusion injury, and that the drug is effective when administered either prior to ischemia or immediately prior to reperfusion.

### Example 5.7

### Ex Vivo Coronary Flow Measurements

Coronary flow was measured in male Mas^{-/-} and Mas^{+/+} mice, and in male Sprague-Dawley rats using Langendorff perfused isolated hearts. Freshly isolated hearts were placed on a Langendorff apparatus (Harvard Apparatus) and perfused at a constant pressure of 80 mmHg with a modified Krebs-Henseleit buffer solution (Sigma K3753) and aerated with 95% oxygen and 5% carbon dioxide, pH 7.35-7.4. The temperature was maintained at 37° C. by surrounding the heart with a water-heated glassware chamber. Coronary flow was measured using a flow measurement system (Harvard Apparatus) which included the transit time flow meter and a flow probe built into an adaptor block located at the perfusate inflow port. Data were recorded continuously using an ISOHEART data acquisition system (Harvard Apparatus). After a 20-min equilibration period, Mas compounds were added to the perfusion buffer reservoir at the desired concentration (agonist AR234960 at 1 μM, or inverse agonist AR244555 at 5 μM) and coronary flow was recorded for 10 min

To determine the role of the Mas-G_{q}-PLC pathway in regulating coronary flow, the Mas inverse agonist AR244555 (5 μM) or the PLC inhibitor U-73122 (0.5 μM) was added to the perfusion buffer for 10 min and then the agonist AR234960 (1 μM) was added to the perfusion buffer. Coronary flow was recorded for another 10 min Changes in coronary flow induced by AR234960 were calculated as percentage of the coronary flow at 10 min following AR234960 treatment, relative to the coronary flow measured immediately prior to addition of AR234960. This protocol allowed for measurement of agonist mediated vasoconstrictor activity, and accounted for changes in baseline coronary flow due to inverse agonist treatment alone.

To determine whether Mas agonist-induced changes in coronary flow were endothelium-dependent, the responses in Langendorff perfused hearts were measured after chemical removal of endothelium with sodium deoxycholate. After a 20-min equilibration period, sodium deoxycholate was added to the perfusion buffer at 0.2 mg/mL for 3 min and then washed out for 10 min Mas compounds were added and coronary flow was recorded for 10 min Adenosine (1 μM), a coronary vasodilator that targets the endothelium, was used as a control to verify effective removal of endothelium.

For ischemia-reperfusion experiments, Langendorff perfused rat hearts were equilibrated for 20 min and then baseline coronary flow was recorded for 10 min Thereafter, all hearts were subjected to 30 min of global ischemia by stopping perfusate flow, followed by 30 min of reperfusion with either vehicle (0.01% DMSO), Mas agonist AR234960 (1 μM) or Mas inverse agonist AR244555 (5 μM) added to the perfusion buffer. Electrocardiography was also continuously recorded during the observation period through electrodes attached directly to the surface of the ventricles to detect cardiac arrhythmias during reperfusion.

Results:

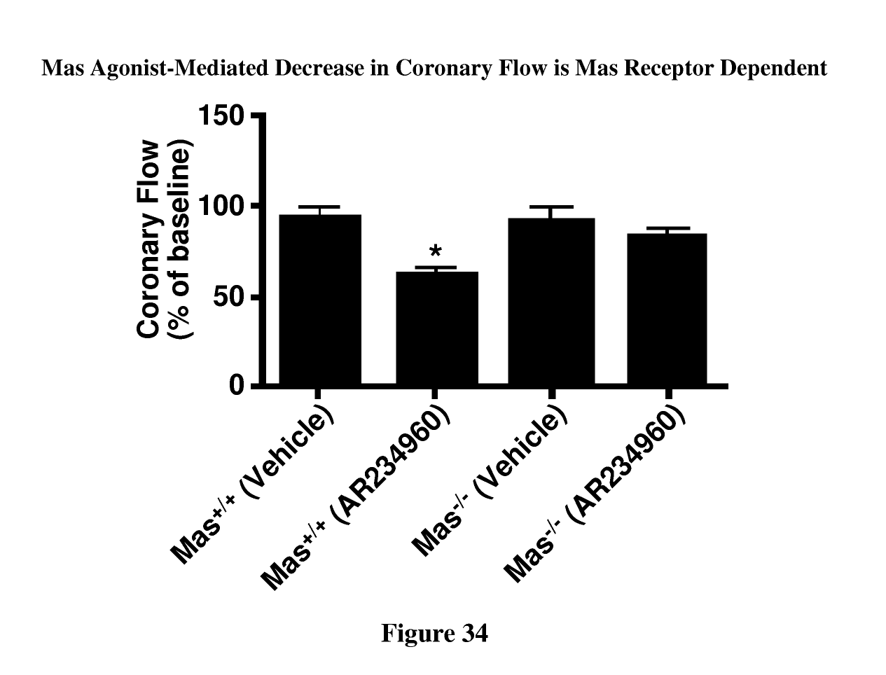

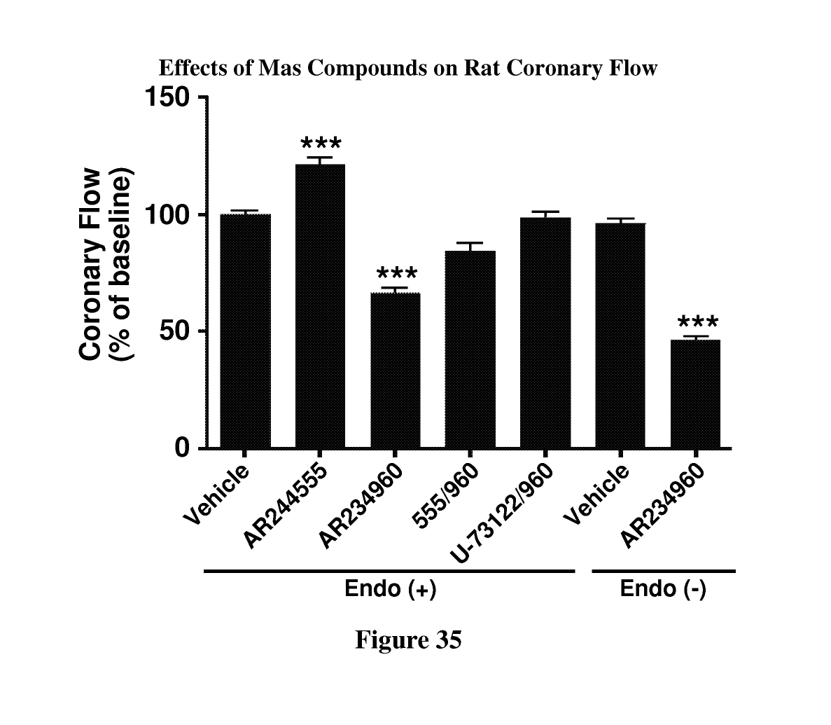

Since Mas expression is enriched in coronary arteries, the experiment was designed to determine whether the Mas receptor plays a role in the regulation of coronary flow. In isolated perfused hearts from genetically altered Mas knockout (Mas^{-/-}) and wild type (Mas^{+/+}) mice there was no detectable difference in coronary flow at baseline (FIG. 34) or after vasoconstriction with Ang II or endothelin-1 (data not shown). However, treatment of Mas^{+/+} mice with the Mas agonist AR234960 resulted in a significant reduction (64% of baseline) in coronary flow. This response was absent in Mas^{-/-} hearts (FIG. 34), indicating that the AR234960-mediated decrease in coronary flow is Mas receptor dependent. A decrease in coronary flow was also observed in isolated perfused rat hearts upon treatment with the agonist AR234960 (FIG. 35). Furthermore, the Mas receptor inverse agonist AR244555 caused a modest but significant increase in coronary flow in rat hearts. Pretreatment with the inverse agonist AR244555 prevented the decrease in coronary flow caused by the agonist AR234960 (FIG. 35). These data demonstrate that agonist stimulation of the Mas receptor causes vasoconstriction whereas inverse agonist treatment reverses the vasoconstriction and promotes dilation of the coronary arteries.

To determine if the Mas agonist-induced decrease in coronary flow was endothelium-mediated or smooth muscle-mediated, the change in coronary flow was measured following treatment with sodium deoxycholate, a chemical that removes the endothelial layer but leaves the smooth muscle intact. To validate this procedure, adenosine was used as an experimental control since it is known to cause vasodilation via activation of adenosine A2 receptors on endothelial cells (de Jong et. al. Pharmacol Ther 87: 141-149 (2000)). The adenosine-mediated increase in coronary flow was abolished after sodium deoxycholate treatment (data not shown), verifying effective removal of endothelium. In contrast, the AR234960-mediated decrease in coronary flow was preserved in hearts denuded of endothelium, (FIG. 35), indicating that vasoconstriction is mediated through Mas receptors on smooth muscle cells. To confirm the role for G_{q}-PLC signaling in the vasoconstriction response, isolated rat hearts were treated with a PLC inhibitor (U-73122) prior to Mas agonist AR234960 treatment. PLC inhibition blocked the decrease in coronary flow caused by AR234960 (FIG. 35).

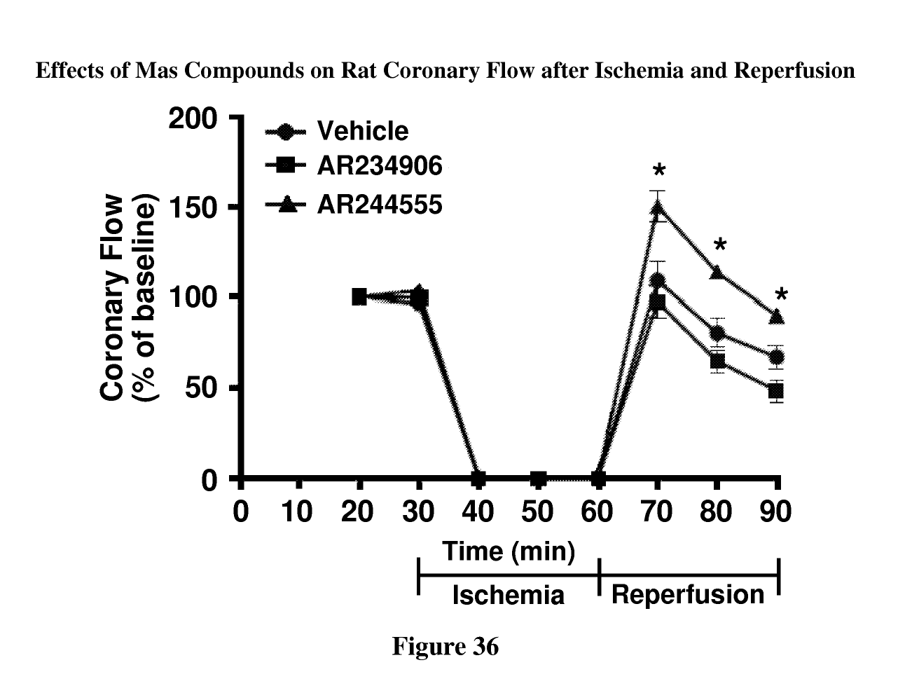

To examine whether Mas activation might promote reperfusion injury following ischemia, isolated perfused rat hearts were subjected to 30 min of global ischemia followed by 30 minutes of reperfusion. During reperfusion, coronary flow in vehicle-treated rats initially returned to pre-ischemia levels but then progressively decreased thereafter (FIG. 36). Treatment with the Mas agonist AR234960 during reperfusion resulted in a trend toward decreased coronary flow during reperfusion. In contrast, treatment of hearts with the Mas inverse agonist AR244555 during reperfusion resulted in significantly elevated coronary flow at all time points during reperfusion compared to vehicle treated hearts. These results suggest that in isolated perfused rat hearts, Mas receptor activity causes decreased coronary flow during reperfusion following ischemia, and that inhibition of Mas during reperfusion can significantly increase coronary flow under these conditions.

Electrocardiography was also continuously recorded during the observation period to detect cardiac arrhythmias during reperfusion. Two out of six hearts (33.3%) in the vehicle group exhibited prolonged (>10 min) ventricular arrhythmias, mainly ventricular fibrillation, during reperfusion. The frequency of arrhythmias was increased to three out of seven hearts (42.9%) with treatment with the Mas agonist AR234960. In contrast, no arrhythmias were observed in the six hearts treated with the Mas inverse agonist AR244555 during reperfusion.

### Example 5.8

### Statistical Analysis

All data are reported as mean+SEM. Statistical significance between two groups was determined using unpaired t-test or using one-way ANOVA followed by Tukey post-hoc test for three or more groups. A p value of <0.05 was considered statistically significant.

### Example 6

### Expression of Mas is Upregulated on Thioglycollate Elicited Peritoneal Mouse Macrophages Following LPS Stimulation

Animals:

Male C57BL6 male mice [25-30 g] (Charles River Laboratories) were housed three per cage and maintained in a humidity controlled room under 12:12 hour light/dark cycles. All animal studies were performed according to the Guide for the Care and Use of Laboratory Animals published by the National Academy of Sciences (1996). All study protocols were reviewed and approved by Arena Pharmaceuticals Institutional Animal Care and Use Committee (IACUC). Water and standard diet was provided ad libitum.

### Preparation of Thioglycollate Peritoneal Macrophages

Mice were injected intraperitoneally, with 5 ml of 3% (w/v) Brewers thioglycollate medium (Difco; sterilized by autoclave). Approximately 5 days, Macrophages were harvested from the peritoneal cavity of euthanized animals by injecting the intact cavity with roughly 5 mL of ice cold RPMI 1640 (+10% fetal calf serum; PSN). Peritoneal macrophages were then collected by spinning at 400×g, 4° C. for 5 minutes. Cells were then seeded in 6 well plates at 1.7×10⁶ cells/ml (2 mL per plate) in RPMI medium and incubated at 5% CO2, 37° C. overnight.

Treatment of Macrophages with LPS:

After plating, overnight, the macrophages were either left in medium, or treated with 1 μg/mL lipopolysaccharide (LPS; SigmaAldrich) for 30, 60, 90, 120, 180, 240, or 360 minutes before harvesting in TRIzol® (Invitrogen). mRNA was prepared using the phenol chloroform method of extraction and the mRNA reverse transcribed into cDNA for qPCR analysis. qPCR was performed for Mas receptor and TNFα and normalized against the house keeping gene beta actin.

Mouse TNFα (f)
5-CACCGTCAGCCATTTGC-3′
Mouse TNFα (r)
5′TTGACGGCAGAGAGGAGGTT-3′
Mouse TNFα (probe)
6FAM-ATCTCATACCAGGAGAAAG-MGBNFQ
Mouse beta-actin (f)
5′-TCCTGGCCTCACTGTCCAC-3′
Mouse beta actin (r)
5′-GGGCCGGACTCATCGTACT-3′
Mouse beta actin probe
VIC-CTGCTTGCTGATCCACATCTGCTGG

Mas 1 gene expression was detected using primer/probe set Mm00434823 (Life technologies).

### Results:

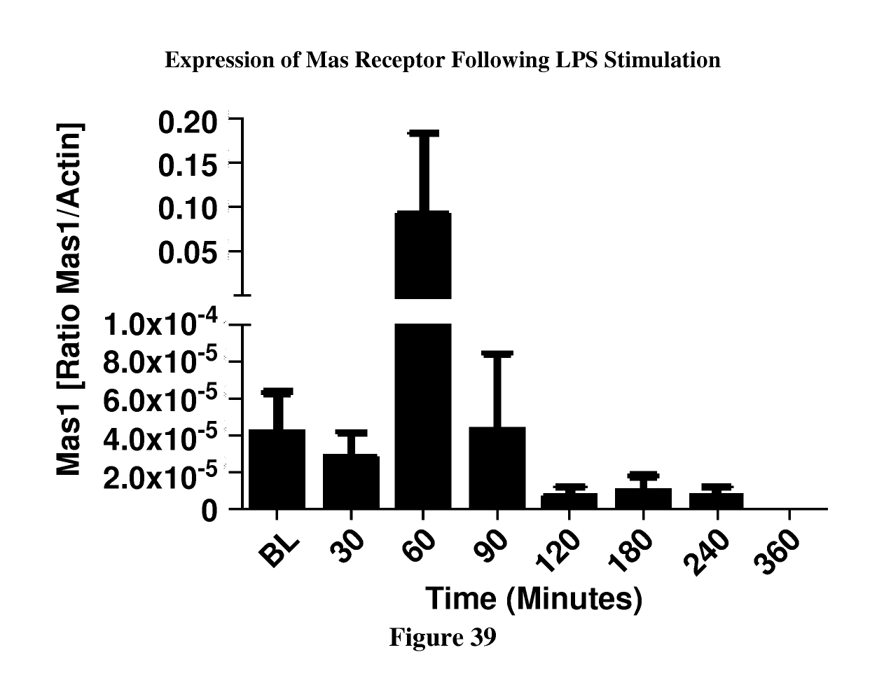

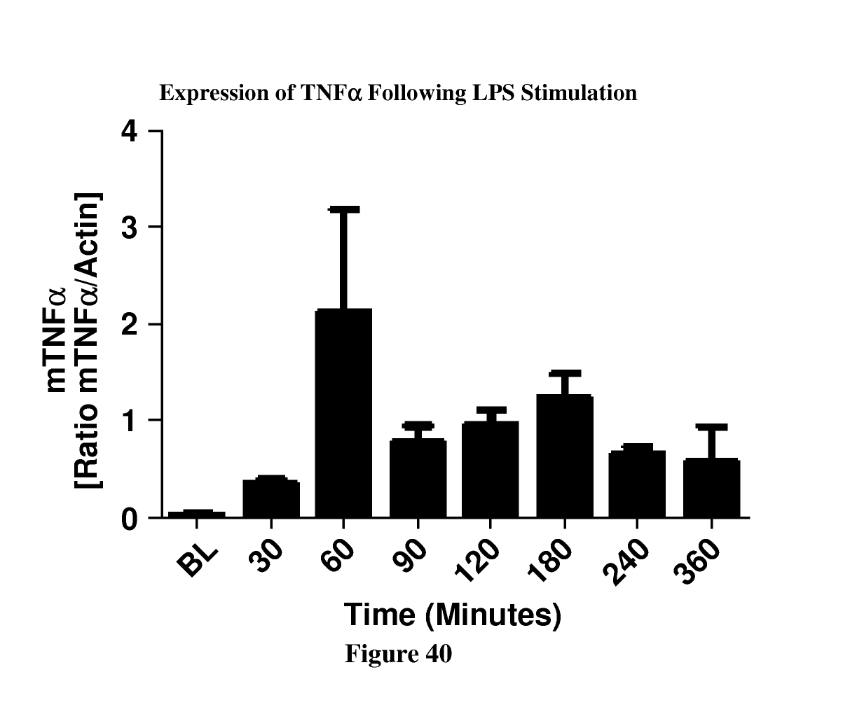

Combined triplicate experiments show that there is a baseline expression of Mas receptor that peaks 1 hour following LPS stimulation. Notably, Mas expression correlates with TNFα expression. This experiment shows that Mas receptor is upregulated in macrophages in response to endotoxin LPS; see FIG. 39 and FIG. 40.

### Example 7

### Mas Receptor Inverse Agonists Suppress LPS Induced TNFα Expression in Mice

Systemic administration of endotoxin, such as lipopolysaccharide (LPS), is a common animal model for sepsis as it induces proinflammatory cytokines, such as TNFα, which correlate with the severity of disease (Rittirsch et. al., J. Leukocyte biology. 81:137-143 (2007)).

Animals:

Male C57BL6 male mice [25-30 g] (Charles River Laboratories) were housed three per cage and maintained in a humidity controlled room under 12:12 hour light/dark cycles. All animal studies were performed according to the Guide for the Care and Use of Laboratory Animals published by the National Academy of Sciences (1996). All study protocols were reviewed and approved by Arena Pharmaceuticals Institutional Animal Care and Use Committee (IACUC). Water and standard diet was provided ad libitum.

LPS Model for Sepsis Induction:

Mice were treated intravenously with either 20% DMSO (vehicle), 1, 3 or 10 mg/kg Compound 170 in 20% DMSO. Alternatively animals were treated with 1 mg/kg IB-MECA in 0.1% DMSO as a positive control (n=6 per group). 1 hour after drug treatment, animals received 500 μg of LPS intraperitoneally Animals were bled 75 minutes after LPS treatment and the blood was spun down for serum. An ELISA for mouse TNFα (Invitrogen) was performed on 1:50 diluted samples the following day.

Result:

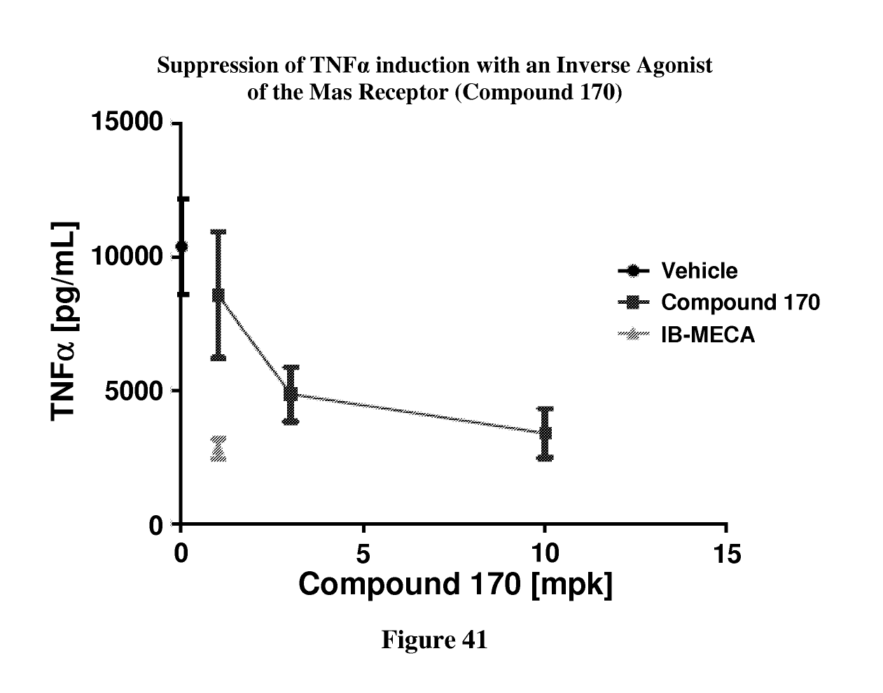

Suppression of TNFα induction with Compound 170 was significant to control treated animals at the 10 and 3 mg/kg dose. The 10 mg/kg dose was not statistically different from the positive control IB-MECA. This experiment demonstrates that Mas inverse agonists can suppress LPS induced TNFα; see FIG. 41.

### Example 8

### Mas receptor Inverse Agonists Suppress Paw Swelling in the Carrageenan-Induced Inflammatory Paw Swelling Model

The carrageenan-induced paw swelling model is associated with elevated levels of proinflammatory cytokines, such as TNFα, that peak at 3 house post-carrageenan injection (Lorman et. al., J. Pain 8(2):127-36 (2007)).

Animals:

Male Sprague Dawley rats (Harlan Laboratories) were housed three per cage and maintained in a humidity controlled room under 12:12 hour light/dark cycles. All animal studies were performed according to the Guide for the Care and Use of Laboratory Animals published by the National Academy of Sciences (1996). All study protocols were reviewed and approved by Arena Pharmaceuticals Institutional Animal Care and Use Committee (IACUC). Water and standard diet was provided ad libitum.

Drug Treatment:

In a blinded and randomized fashion, rats were intraperitoneally dosed with a Mas receptor inverse agonist (AR305352, N-(4-chloro-2-(4-(3,3,3-trifluoropropyl)piperazin-1-yl)phenyl)-2,6-difluorobenzamide, see Zhang, T., et. al., Am J Physiol Heart Circ Physiol 302:H299-H311, (2012)) at 1, 3 and 10 mg/kg, 20% HPCD or 2 mg/kg of dexamethasone, 30 minutes prior to injection of 100 μl of saline or 100 μg of λ-carrageenan into the left or right foot pad of anesthetized animals. The difference in footpad width was measured using a caliper at 1, 2, 3, 6 and 24 hours post-injection.

Results:

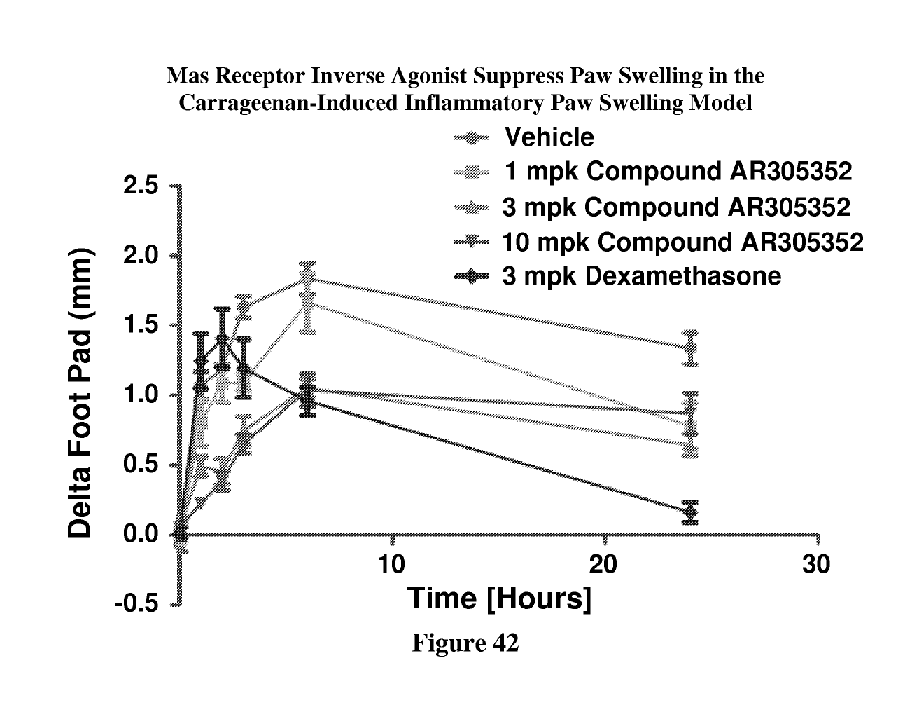

Duplicate experiments with the Mas receptor inverse agonist, AR305352, demonstrated that pre-treatment with Mas inverse agonists suppress foot-pad inflammation in a dose-dependent manner at all time points for the 3 and 10 mg/kg dose for up to 24 hours. The positive control, 2 mg/kg of dexamethasone, inhibited swelling from 3 hours onwards which is consistent with its pharmacokinetics. This experiment demonstrates that Mas inverse agonists can suppress inflammation in the carrageenan foot-pad model; see FIG. 42.

### Example 9

### Mas Inverse Agonist (Compound 170) can Protect Kidneys Subjected to Ischemia Reperfusion Injury

Animals:

Male Sprague-Dawley Rats (Charles River Laboratories) were maintained in a humidity controlled room under 12:12 hour light/dark cycles. All animal studies were performed according to the Guide for the Care and Use of Laboratory Animals published by the National Academy of Sciences (1996). Water and standard diet was provided ad libitum.

Ischemia-Reperfusion Injury Model:

Rats were anesthetized with pentobarbital (70 mg/kg). Normal body temperature was maintained by placing the animals on heating pads until recovery from anesthesia. Following a midline abdominal incision, the left renal pedicle was localized and the renal artery and vein were dissected. An atraumatic microvascular clamp was placed, and the left renal artery was occluded for 45 minutes. After inspection for signs of ischemia, the wound was covered with PBS soaked cotton. After release of the clamp, restoration of blood-flow was inspected visually and the wound was closed with surgical staples and the animal was allowed to recover. 24 hours following the end of ischemia blood was collected and markers of kidney function, creatinine and blood urea nitrogen (BUN) were measured.

Treatment of Animals with an Mas Inverse Agonist (Compound 170):

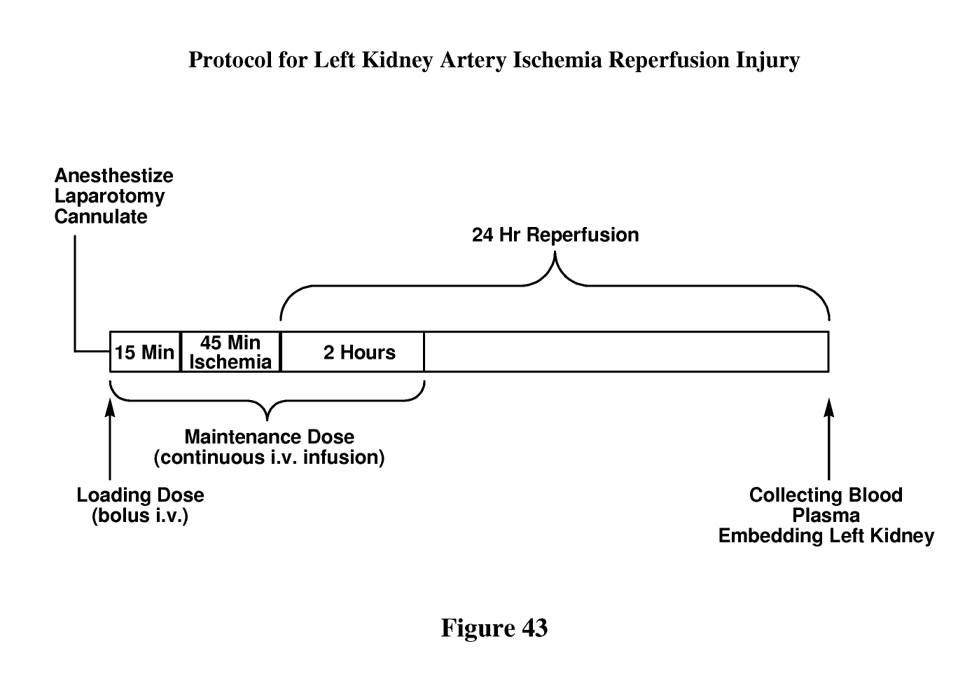

15 minutes prior to renal artery occlusion either vehicle (20% hydroxypropyl-β-cyclodextrin) or a loading dose of 6.26 mg/kg Mas inverse agonist (Compound 170) was administered i.v. followed by a i.v. maintenance dose of 1.64 mg/kg/hr lasting for 2 hours following removal of the renal artery clamp. A renoprotective positive control peptide, Atrial natriuretic peptide (ANP, 0.2 μg/kg/min), was administered by continuous i.v. infusion starting 15 minutes prior to renal artery occlusion and lasting for 2 hours following removal of the renal artery clamp, see FIG. 43 for protocol.

Results:

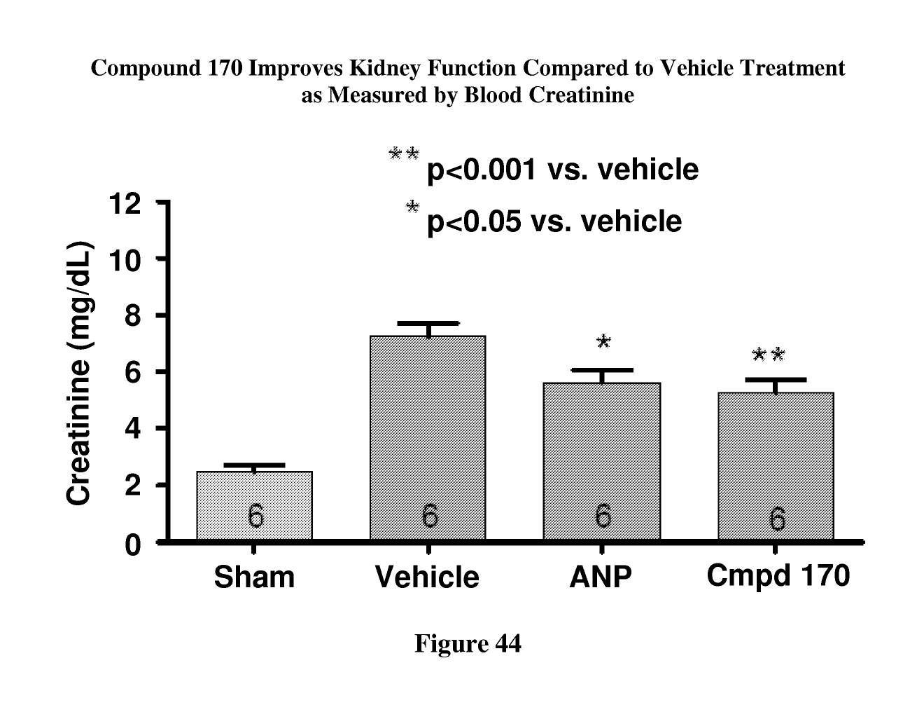

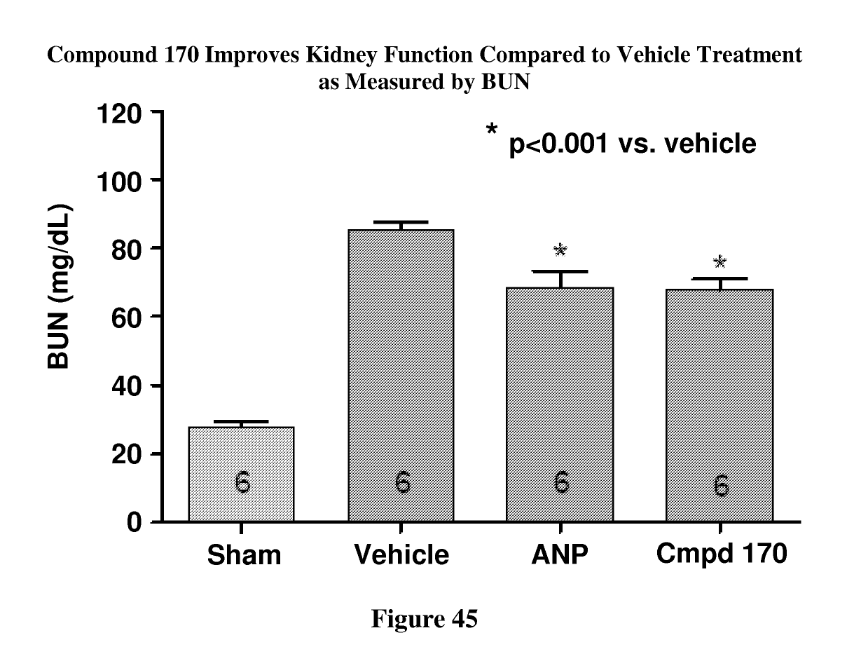

The data demonstrate that administration of the Mas inverse agonist, Compound 170, improves kidney function compared to vehicle treatment as measured by blood creatinine (FIG. 44) and BUN levels (FIG. 45). The degree of renal protection is at the same activity compared to ANP which is known to be renoprotective in this model (Chujo, K. et. al., J. Biosci. Bioeng. June, 109(6):526-30 (2010)) and in human clinical studies (Nigwekar, S. U., Cochrane Database Syst. Rev. October 7, (4):CD006028 (2009)).

### Example 10

### Mas Receptor Inverse Agonist (Compound 170) Reduces Brain Damage in a Rat Model of Stroke

Animals:

Male Sprague Dawley rats (Charles River Laboratories) were housed three per cage and maintained in a humidity controlled room under 12:12 hour light/dark cycles. All animal studies were performed according to the Guide for the Care and Use of Laboratory Animals published by the National Academy of Sciences (1996). All study protocols were reviewed and approved by Arena Pharmaceuticals Institutional Animal Care and Use Committee (IACUC). Water and standard diet was provided ad libitum.

Rat Model of Transient Cerebral Ischemia/Stroke:

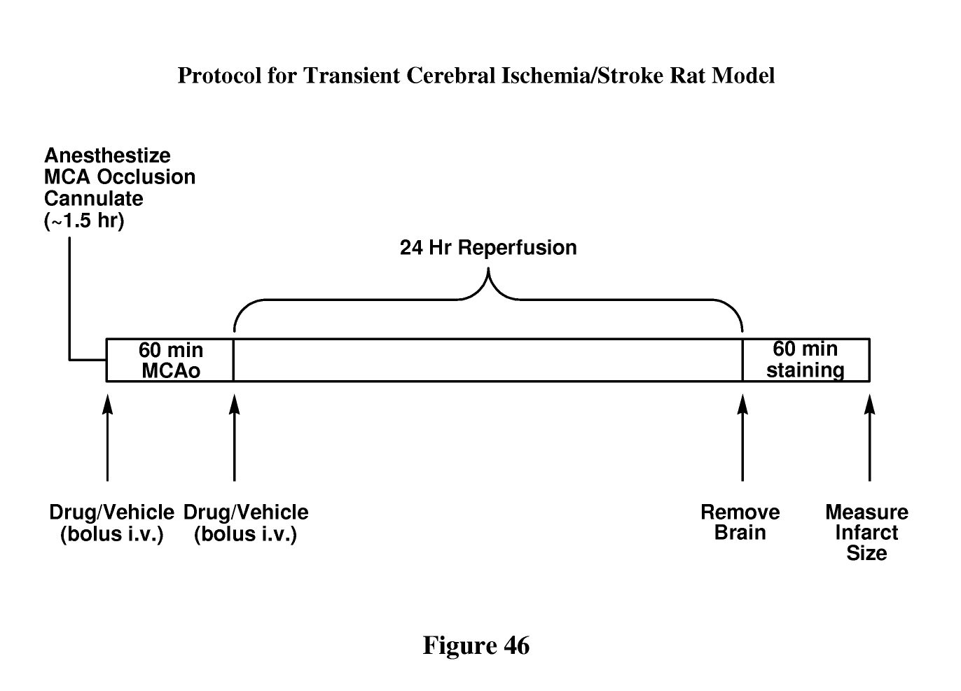

Rats were anesthetized with pentobarbital (70 mg/kg). Normal body temperature was maintained by placing the animals on heating pads. A midline neck incision was made and soft tissues were pulled apart, and the external carotid artery (ECA), and internal carotid artery (ICA) were exposed. The ECA was ligated with a 5-0 silk suture and the ICA was temporarily blocked by a microvascular clip. An incision was made around the left hind leg area and exposing the femoral vein. A 30-mm length of 3-0 monofilament nylon suture coated with 0.1% poly-lysine was inserted into the right ICA lumen through a small incision at about 4-mm proximal to the carotid bifurcation. The filament was advanced 18-22 mm from the start of the carotid bifurcation to block the origin of the right middle cerebral artery (MCA) for the ischemia time designed. Reperfusion was obtained by pulling out the occluding filament at the end of the MCA. After occlusion the tissues around the neck were closed with 4-0 silk suture (Ethicon, 1677G). The skin around the neck was closed with metal clips and the cutting area was treated with iodine. At the end of the reperfusion period (e.g. 24 h) the rats were anesthetized with Nembutal (70 mg/kg) and the brains were removed. Brains were then sectioned coronally with a razor blade at 2-mm intervals and incubated for 60 min in a 2% solution of 2,3,5 triphenyltetrazolium chloride (TTC) at 37° C. for vital staining. Whole area (WA) and infarct area (IA) were measured for each coronal section. Brain damage was assessed by IA/(IA+WA). See FIG. 46 for protocol.

Treatment of Animals with Mas Inverse Agonists:

A bolus i.v. dose of vehicle (20% hydroxypropyl-β-cyclodextrin) or 3 mg/kg Mas inverse agonist (Compound 170) was administered either 1 min prior to MCA occlusion or at the onset of reperfusion. A known neuroprotective drug Tacrolimus (FK506, 0.32 mg/min i.v. bolus) was administered in the same fashion as a positive control (Bochelen D. et. al., J Pharmacol Exp Ther. February, 288(2):653-9 (1999)).

Result:

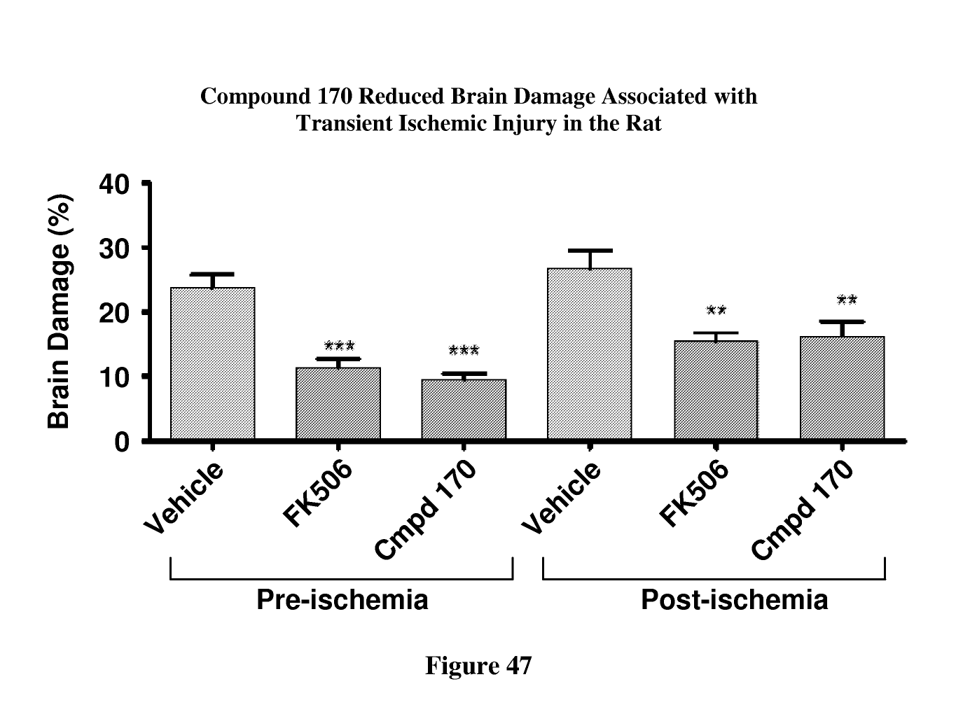

Inhibition of Mas G-protein signaling with the Mas Inverse Agonist (Compound 170) reduced brain damage associated with transient ischemic injury in the rat (FIG. 47).

Those skilled in the art will recognize that various modifications, additions, and substitutions to the illustrative examples set forth herein can be made without departing from the spirit of the invention and are, therefore, considered within the scope of the invention.

1. A compound selected from compounds of Formula (I) and pharmaceutically acceptable salts, solvates, and hydrates thereof: 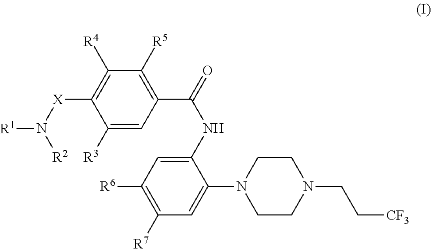
wherein:
X is CH₂ or CH₂CH₂; or X is absent;
R⁴, R⁵, R⁶, and R⁷ are each selected independently from: H and halogen; and
(A) R¹ is selected from: H, C₁-C₆ alkyl, C₁-C₆-alkyl-O—C₁-C₆-alkyl, C₃-C₇ cycloalkyl, C₁-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy;
R² is selected from: H and C₁-C₆ alkyl, wherein said C₁-C₆ alkyl is optionally substituted with one or more substituents selected from: hydroxyl and cyano; and
R³ is selected from: H and halogen; or
(B) R¹ and R² together with the nitrogen atom to which they are both bonded form a group selected from: heteroaryl and heterocyclyl, each optionally substituted with one or more substituents selected from: C₁-C₆ alkoxycarbonyl, C₁-C₆ alkoxycarbonylamino, C₁-C₆ alkyl, C₁-C₆ alkylcarboxamide, C₁-C₆ alkylsulfonyl, amino, C₃-C₇ cycloalkyl, C₁-C₁₃ cycloalkylalkyl, carboxamide, carboxyl, C₂-C₆ dialkylamino, C₂-C₆ dialkylcarboxamide, heteroaryl-C₁-C₆-alkyl, heterocyclyl, heterocyclyl-C₁-C₆-alkyl, hydroxyl, hydroxyheterocyclyl, and oxo, wherein said C₁-C₆ alkyl and C₁-C₆ alkylcarboxamide are each optionally substituted with one or more substituents selected from: carboxyl, hydroxyl, and oxo; and
R³ is selected from: H and halogen; or
(C) R¹ is selected from: H, C₁-C₆ alkyl, C₁-C₆-alkyl-O—C₁-C₆-alkyl, C₃-C₇ cycloalkyl, C₁-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy; and
R² and R³ together form CH₂.

2. The compound according to claim 1, wherein:
R¹ is selected from: H, C₁-C₆ alkyl, C₁-C₆-alkyl-O—C₁-C₆-alkyl, C₃-C₇ cycloalkyl, C₁-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy;
R² is selected from: H and C₁-C₆ alkyl, wherein said C₁-C₆ alkyl is optionally substituted with one or more substituents selected from: hydroxyl and cyano; and
R³ is selected from: H and halogen.

3. The compound according to claim 1, wherein:
R¹ is selected from: H, ethyl, 2-hydroxyethyl, 3-(1H-imidazol-1-yl)propyl, 4-methylpyridin-3-yl, methyl, 2-cyanoethyl, 2-amino-2-oxoethylamino, (1-methylpiperidin-4-yl)methyl, cyanomethyl, 1-amino-1-oxopropan-2-yl, 1,1-dioxo-tetrahydrothiophen-3-yl, 1-hydroxy-4-methylpentan-2-yl, 2-(1H-imidazol-5-yl)ethyl, (1-methyl-1H-imidazol-5-yl)methyl, 2-carbamoylcyclohexyl, 3-hydroxy-1-methoxy-1-oxopropan-2-yl, 1,3-dihydroxypropan-2-yl, 1-amino-3-hydroxy-1-oxopropan-2-yl, 2-hydroxycyclohexyl, 2-oxoazepan-3-yl, 2-(2-oxoimidazolidin-1-yl)ethyl, pyrrolidin-2-ylmethyl, pyrrolidin-3-yl, piperidin-3-yl, piperidin-4-yl, 2-hydroxypropyl, 2-hydroxypyridin-3-yl, 2-(4-methylpiperazin-1-yl)ethyl, 1-hydroxypropan-2-yl, 1,3-dihydroxy-2-(hydroxymethyl)propan-2-yl, 2-acetamidoethyl, 1-hydroxybutan-2-yl, 2-(1-methylpyrrolidin-2-yl)ethyl, 2-(dimethylamino)ethyl, 2-morpholinoethyl, 1-ethyl-2-oxoazepan-3-yl, 3-(dimethylamino)tetrahydrothiophen-3-yl)methyl, 2-(diethylamino)ethyl, 1-hydroxy-3-methylpentan-2-yl, 5-aminopentyl, 3-amino-1-imino-3-oxopropyl, (1-hydroxycyclohexyl)methyl, 2-(hydroxymethyl)pyrrolidin-1-yl)ethyl, 2-methyl-2-(piperidin-1-yl)propyl, benzyl, 2-(methylsulfinyl)ethyl, 2-(azepan-1-yl)ethyl, 3-hydroxybutyl, 1-amino-3-methyl-1-oxobutan-2-yl, 2-(2-(2-aminoethoxy)ethoxy)ethyl, 2-(hydroxymethyl)pyrrolidin-1-yl, 1,3-dihydroxybutan-2-yl, 2-morpholino-2-oxoethyl, 2-(dimethylamino)-2-(pyridin-3-yl)ethyl, 2-(pyrrolidin-1-yl)ethyl, 3-amino-1-methoxy-1-oxopropan-2-yl, 4-amino-1-methoxy-1-oxobutan-2-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, 2-hydroxycyclopentyl, piperidine-4-carbonyl, 2-aminocyclohexanecarbonyl, morpholine-2-carbonyl, 3-aminopropanoyl, 2-aminoacetyl, 4-hydroxypyrrolidine-2-carbonyl, 2-aminopropanoyl, 2-amino-3-hydroxypropanoyl, 2-hydroxyacetyl, thiomorpholine-3-carbonyl, pyrrolidine-2-carbonyl, 2-(morpholin-4-yl)acetyl, 2-(1H-tetrazol-5-yl)acetyl, 2-(dimethylamino)acetyl, 3-oxo-2,3-dihydroisoxazole-5-carbonyl, 6-oxo-1,6-dihydropyridazine-3-carbonyl, 2,4-dihydroxypyrimidine-5-carbonyl, 5-oxo-4,5-dihydro-1H-1,2,4-triazole-3-carbonyl, 4-aminotetrahydro-2H-thiopyran-4-carbonyl, 2-(3-amino-2-oxopyrrolidin-1-yl)acetyl, 6-hydroxynicotinoyl, 2-hydroxynicotinoyl, 2,6-dihydroxyisonicotinoyl, 2,6-dioxo-1,2,3,6-tetrahydropyrimidine-4-carbonyl, 5-hydroxy-1-methyl-1H-pyrazole-3-carbonyl, 3-(3-hydroxyisoxazol-4-yl)propanoyl, 3-carboxypropanoyl, 5-hydroxypyrazine-2-carbonyl, 6-hydroxypicolinoyl, 4-methylmorpholine-2-carbonyl, 4-ethylmorpholine-2-carbonyl, 4-(2-hydroxyethyl)morpholine-2-carbonyl, 4-(3,3-dimethylbutyl)morpholine-2-carbonyl, 4-(2-hydroxyethyl)morpholine-3-carbonyl, 4-ethylmorpholine-3-carbonyl, 4-(2-hydroxyethyl)thiomorpholine-3-carbonyl, 4-ethylthiomorpholine-3-carbonyl, 3-hydroxypropanoyl, 4-hydroxycyclohexanecarbonyl, 3-hydroxypentanoyl, 2-hydroxy-2-methylpropanoyl, 1-hydroxycyclopropanecarbonyl, 3-hydroxybutanoyl, 3-hydroxy-2,2-dimethylpropanoyl, 4-hydroxybutanoyl, 2-ethyl-2-hydroxybutanoyl, 2-hydroxycyclohexanecarbonyl, 2-cyclohexyl-2-hydroxyacetyl, 3-hydroxy-3-methylbutanoyl, 2-hydroxy-4-methylpentanoyl, 1-(tert-butoxycarbonyl)-4-hydroxypyrrolidine-2-carbonyl, 4-(tert-butoxycarbonyl)thiomorpholine-3-carbonyl, 2-(1-(tert-butoxycarbonyl)piperidin-2-yl)acetyl, 3-hydroxy-2-(hydroxymethyl)-2-methylpropanoyl, 2-(piperidin-2-yl)acetyl, 4-(hydroxymethyl)cyclohexanecarbonyl, 3-(dimethylamino)propanoyl, 2-(pyrrolidin-3-yl)acetyl, 3-(piperidin-1-yl)propanoyl, 4-aminocyclohexanecarbonyl, pyrrolidine-3-carbonyl, 3-(diethylamino)propanoyl, 2-(4-aminocyclohexyl)acetyl, 3-morpholinopropanoyl, 1-methylpiperidine-4-carbonyl, 3-aminocyclohexanecarbonyl, 2-amino-4-carboxybutanoyl, 4-amino-4-carboxybutanoyl, 3-aminocyclopentanecarbonyl, 1-methylpiperidine-3-carbonyl, 2-(piperidin-3-yl)acetyl, azetidine-3-carbonyl, 2-(4-(hydroxymethyl)piperidin-1-yl)acetyl, 2-(3-hydroxypiperidin-1-yl)acetyl, 2-(piperazin-1-yl)acetyl, 2-(3-aminopyrrolidin-1-yl)acetyl, 2-(2-(hydroxymethyl)morpholino)acetyl, 2-(4-propylpiperazin-1-yl)acetyl, 2-(5-oxo-1,4-diazepan-1-yl)acetyl, 2-(4-carbamoylpiperidin-1-yl)acetyl, 2-(2-carbamoylpyrrolidin-1-yl)acetyl, 2-(4-(dimethylamino)piperidin-1-yl)acetyl, 2-(3-(dimethylamino)pyrrolidin-1-yl)acetyl, 2-(4-hydroxypiperidin-1-yl)acetyl, 2-(2,5-diazabicyclo[2.2.1]heptan-2-yl)acetyl, 2-(hexahydropyrrolo[1,2-a]pyrazin-2(1H)-yl)acetyl, 2-(3-(hydroxymethyl)piperidin-1-yl)acetyl, 2-(3-methylpiperazin-1-yl)acetyl, 2-(4-methylpiperidin-1-yl)acetyl, 2-(3-oxopiperazin-1-yl)acetyl, 2-(4-carbamoylpiperazin-1-yl)acetyl, 2-(3-methylpiperidin-1-yl)acetyl, 2-(4-methylpiperazin-1-yl)acetyl, 2-(4-ethylpiperazin-1-yl)acetyl, 2-(2-(2-hydroxyethyl)piperidin-1-yl)acetyl, 2-(3-hydroxypyrrolidin-1-yl)acetyl, 2-(2-(hydroxymethyl)pyrrolidin-1-yl)acetyl, 2-(3-carbamoylpiperidin-1-yl)acetyl, 4-(phosphonooxy)cyclohexanecarbonyl, 2-(phosphonooxy)acetyl, 3-(tert-butoxycarbonylamino)pyrrolidine-1-carbonyl, 2-amino-4-methylpentanoyl, 2-amino-3-cyanopropanoyl, 4-amino-1,1-dioxotetrahydro-2H-thiopyran-4-carbonyl, 2,4-diamino-4-oxobutanoyl, 3-amino-2-hydroxypropanoyl, 2-hydroxypropanoyl, 5-(hydroxymethyl)-1H-1,2,3-triazole-4-carbonyl, piperazine-1-carbonyl, 4-ethylpiperazine-1-carbonyl, 1,1-dioxotetrahydro-2H-thiopyran-4-carbonyl, 3-hydroxypyrrolidine-1-carbonyl, 4-(2-hydroxyethyl)piperazine-1-carbonyl, 4-(hydroxymethyl)piperidine-1-carbonyl, 3-aminopiperidine-1-carbonyl, 3-hydroxyazetidine-1-carbonyl, 3-aminopyrrolidine-1-carbonyl, 2-carbamoylpyrrolidine-1-carbonyl, 4-(dimethylamino)piperidine-1-carbonyl, 4-carbamoylpiperazine-1-carbonyl, 3-oxopiperazine-1-carbonyl, 2-(hydroxymethyl)pyrrolidine-1-carbonyl, 2-(2-hydroxyethyl)piperidine-1-carbonyl, 2-(hydroxymethyl)morpholine-4-carbonyl, 3-carboxyazetidine-1-carbonyl, 4-(3-hydroxypropyl)piperidine-1-carbonyl, 3-hydroxypiperidine-1-carbonyl, 4-cyanopiperidine-1-carbonyl, 2-(hydroxymethyl)piperidine-1-carbonyl, 4-hydroxypiperidine-1-carbonyl, 2-oxopyrrolidine-1-carbonyl, 3-(hydroxymethyl)piperidine-1-carbonyl, 3-(hydroxymethyl)pyrrolidine-1-carbonyl, 3-(phosphonooxy)pyrrolidine-1-carbonyl, 1-(tert-butoxycarbonyl)piperidine-4-carbonyl, 2-(tert-butoxycarbonylamino)-cyclohexanecarbonyl, 1-(tert-butoxycarbonyl)piperidine-3-carbonyl, 3-(tert-butoxycarbonylamino)piperidine-1-carbonyl, 4-(tert-butoxycarbonyl)morpholine-2-carbonyl, 3-(tert-butoxycarbonylamino)propanoyl, 2-(tert-butoxycarbonylamino)acetyl, 3-(tert-butoxycarbonylamino)-2-hydroxypropanoyl, 2-(tert-butoxycarbonylamino)propanoyl, 2-(tert-butoxycarbonylamino)-3-hydroxypropanoyl, 1-(tert-butoxycarbonyl)pyrrolidine-2-carbonyl, 4-(tert-butoxycarbonylamino)tetrahydro-2H-thiopyran-4-carbonyl, 4-tert-butoxy-4-oxobutanoyl, 2-(3-(tert-butoxycarbonylamino)-2-oxopyrrolidin-1-yl)acetyl, 4-amino-2-(tert-butoxycarbonylamino)-4-oxobutanoyl, 2-(1-(tert-butoxycarbonyl)pyrrolidin-3-yl)acetyl, 4-(tert-butoxycarbonylamino)cyclohexanecarbonyl, 1-(tert-butoxycarbonyl)pyrrolidine-3-carbonyl, 2-(4-(tert-butoxycarbonylamino)cyclohexyl)acetyl, 3-(tert-butoxycarbonylamino)cyclohexanecarbonyl, 2-(tert-butoxycarbonylamino)-4-carboxybutanoyl, 4-(tert-butoxycarbonylamino)-4-carboxybutanoyl, 3-(tert-butoxycarbonylamino)cyclopentanecarbonyl, 2-(1-(tert-butoxycarbonyl)piperidin-3-yl)acetyl, 1-(tert-butoxycarbonyl)azetidine-3-carbonyl, 2-(3-(tert-butoxycarbonylamino)pyrrolidin-1-yl, 2-(4-(tert-butoxycarbonyl)-3-methylpiperazin-1-yl)acetyl, 2-(tert-butoxycarbonylamino)-4-methylpentanoyl, 2-(tert-butoxycarbonylamino)-3-cyanopropanoyl, and 4-(tert-butoxycarbonylamino)-1,1-dioxotetrahydro-2H-thiopyran-4-carbonyl;
R² is selected from: H, ethyl, methyl, isopropyl, 2-hydroxyethyl, 2-cyanoethyl, and tert-butyl; and
R³ is selected from: H and chloro.

4. The compound according to claim 1, wherein:
R¹ is selected from: H, ethyl, 2-hydroxyethyl, 3-(1H-imidazol-1-yl)propyl, 4-methylpyridin-3-yl, methyl, 2-cyanoethyl, 2-amino-2-oxoethylamino, (1-methylpiperidin-4-yl)methyl, cyanomethyl, 1-amino-1-oxopropan-2-yl, 1,1-dioxo-tetrahydrothiophen-3-yl, 1-hydroxy-4-methylpentan-2-yl, 2-(1H-imidazol-5-yl)ethyl, (1-methyl-1H-imidazol-5-yl)methyl, 2-carbamoylcyclohexyl, 3-hydroxy-1-methoxy-1-oxopropan-2-yl, 1,3-dihydroxypropan-2-yl, 1-amino-3-hydroxy-1-oxopropan-2-yl, 2-hydroxycyclohexyl, 2-oxoazepan-3-yl, 2-(2-oxoimidazolidin-1-yl)ethyl, pyrrolidin-2-ylmethyl, pyrrolidin-3-yl, piperidin-3-yl, piperidin-4-yl, 2-hydroxypropyl, 2-hydroxypyridin-3-yl, 2-(4-methylpiperazin-1-yl)ethyl, 1-hydroxypropan-2-yl, 1,3-dihydroxy-2-(hydroxymethyl)propan-2-yl, 2-acetamidoethyl, 1-hydroxybutan-2-yl, 2-(1-methylpyrrolidin-2-yl)ethyl, 2-(dimethylamino)ethyl, 2-morpholinoethyl, 1-ethyl-2-oxoazepan-3-yl, 3-(dimethylamino)tetrahydrothiophen-3-yl)methyl, 2-(diethylamino)ethyl, 1-hydroxy-3-methylpentan-2-yl, 5-aminopentyl, 3-amino-1-imino-3-oxopropyl, (1-hydroxycyclohexyl)methyl, 2-(hydroxymethyl)pyrrolidin-1-yl)ethyl, 2-methyl-2-(piperidin-1-yl)propyl, benzyl, 2-(methylsulfinyl)ethyl, 2-(azepan-1-yl)ethyl, 3-hydroxybutyl, 1-amino-3-methyl-1-oxobutan-2-yl, 2-(2-(2-aminoethoxy)ethoxy)ethyl, 2-(hydroxymethyl)pyrrolidin-1-yl, 1,3-dihydroxybutan-2-yl, 2-morpholino-2-oxoethyl, 2-(dimethylamino)-2-(pyridin-3-yl)ethyl, 2-(pyrrolidin-1-yl)ethyl, 3-amino-1-methoxy-1-oxopropan-2-yl, 4-amino-1-methoxy-1-oxobutan-2-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, and 2-hydroxycyclopentyl;
R² is selected from: H, ethyl, methyl, isopropyl, 2-hydroxyethyl, 2-cyanoethyl, and tert-butyl; and
R³ is selected from: H and chloro.

5. The compound according to claim 1, wherein:
R¹ and R² together with the nitrogen atom to which they are both bonded form a group selected from: heteroaryl and heterocyclyl, each optionally substituted with one or more substituents selected from: C₁-C₆ alkoxycarbonyl, C₁-C₆ alkoxycarbonylamino, C₁-C₆ alkyl, C₁-C₆ alkylcarboxamide, C₁-C₆ alkylsulfonyl, amino, C₃-C₇ cycloalkyl, C₁-C₁₃ cycloalkylalkyl, carboxamide, carboxyl, C₂-C₆ dialkylamino, C₂-C₆ dialkylcarboxamide, heteroaryl-C₁-C₆-alkyl, heterocyclyl, heterocyclyl-C₁-C₆-alkyl, hydroxyl, hydroxyheterocyclyl, and oxo, wherein said C₁-C₆ alkyl and C₁-C₆ alkylcarboxamide are each optionally substituted with one or more substituents selected from: carboxyl, hydroxyl, and oxo; and
R³ is selected from: H and halogen.

6. The compound according to claim 1, wherein:
R¹ and R² together with the nitrogen atom to which they are both bonded form a group selected from: 1,1-dioxo-thiomorpholin-4-yl, 3-hydroxypyrrolidin-1-yl, 4-(2-hydroxyethyl)piperazin-1-yl, hexahydropyrrolo[1,2-a]pyrazin-2(1H)-yl, 4-ethylpiperazin-1-yl, piperidin-1-yl, 1H-imidazol-1-yl, morpholino, 4-methylpiperazin-1-yl, pyrrolidin-1-yl, 1H-1,2,4-triazol-1-yl, 1H-pyrazol-1-yl, 1H-pyrrol-1-yl, 2H-tetrazol-5-yl, piperazin-1-yl, 4-(dimethylamino)piperidin-1-yl, 4-(hydroxymethyl)piperidin-1-yl, 2-carbamoylpyrrolidin-1-yl, 2-(2-hydroxyethyl)piperidin-1-yl, 4-carbamoylpiperazin-1-yl, 3-oxopiperazin-1-yl, 4-(2-cyclohexylethyl)piperazin-1-yl, 2,7-diazaspiro[4.4]nonan-2-yl, 3-(methylsulfonyl)pyrrolidin-1-yl, 6,7-dihydro-1H-imidazo[4,5-c]pyridin-5(4H)-yl, 2-(hydroxymethyl)piperidin-1-yl, 3-aminopyrrolidin-1-yl, 2-methylpiperazin-1-yl, 3-aminopiperidin-1-yl, 4-aminopiperidin-1-yl, 2-carbamoylpiperidin-1-yl, 5,6-dihydropyrimidin-1(4H)-yl, 4-hydroxy-2-(methoxycarbonyl)pyrrolidin-1-yl, 4-hydroxypiperidin-1-yl, 4-(2-(pyridin-2-yl)ethyl)piperazin-1-yl, 3-hydroxypiperidin-1-yl, 3-(diethylcarbamoyl)piperidin-1-yl, 2,3,4,6,7,8-hexahydro-1H-pyrimido[1,2-a]pyrimidin-1-yl, 4-cyclopentylpiperazin-1-yl, 1,4-oxazepan-4-yl, 2-(pyrrolidin-1-ylmethyl)pyrrolidin-1-yl, 4-morpholinopiperidin-1-yl, 4-(cyclohexylmethyl)piperazin-1-yl, 4-oxopiperidin-1-yl, 4-acetylpiperazin-1-yl, 1,4′-bipiperidin-1′-yl, 4-(ethoxycarbonyl)piperidin-1-yl, 2-(hydroxymethyl)morpholino, 2-(hydroxymethyl)pyrrolidin-1-yl, 3-hydroxyazetidin-1-yl, 4-hydroxy-1,4′-bipiperidin-1′-yl, 3-(hydroxymethyl)piperidin-1-yl, 2,5-diazabicyclo[2.2.1]heptan-2-yl, 5-oxo-1,4-diazepan-1-yl, 4-(2-hydroxyethyl)piperidin-1-yl, 3-(carboxymethyl)pyrrolidin-1-yl, 2,7-diazaspiro[3.5]nonan-2-yl, 4-(tert-butoxycarbonyl)-2-(carboxymethyl)piperazin-1-yl, 4-(tert-butoxycarbonyl)-2-carboxypiperazin-1-yl, 4-carboxypiperidin-1-yl, 2-(carboxymethyl)morpholino, 2-(carboxymethyl)piperazin-1-yl, 2-carboxypiperazin-1-yl, 4-(carboxymethyl)piperazin-1-yl, 2-carboxy-5,6-dihydroimidazo[1,2-a]pyrazin-7(8H)-yl, 2-carbamoylpiperazin-1-yl, 2-(methylcarbamoyl)piperazin-1-yl, 2-(2-hydroxyethylcarbamoyl)piperazin-1-yl, 2-(1-hydroxypropan-2-ylcarbamoyl)piperazin-1-yl, 3-carbamoylpiperidin-1-yl, 4-carbamoylpiperidin-1-yl, 3-(hydroxymethyl)pyrrolidin-1-yl, 2-oxopyrrolidin-1-yl, 2,5-dioxoimidazolidin-1-yl, 2,6-dioxotetrahydropyrimidin-1(2H)-yl, 3-methyl-2,5-dioxoimidazolidin-1-yl, and 4-isobutyl-2,5-dioxoimidazolidin-1-yl; and
R³ is H.

7. The compound according to claim 1, wherein:
R¹ is selected from: H, C₁-C₆ alkyl, C₁-C₆-alkyl-O—C₁-C₆-alkyl, C₃-C₇ cycloalkyl, C₁-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy; and
R² and R³ together form CH₂.

8. The compound according to claim 1, wherein:
R¹ is selected from: H, methyl, butyl, 3-hydroxypropyl, 3,3-dimethylbutyl, (tetrahydro-2H-pyran-4-yl)methyl, 2-methoxyethyl, 3-amino-3-oxopropyl, 2-hydroxyethyl, 2-ethoxy-2-oxoethyl, 2-amino-2-oxoethyl, cyanomethyl, 2-ethoxyethyl, 2-(diethylamino)ethyl, 2-(methylsulfonyl)ethyl, butyr-1-yl, 2-ethylbutanoyl, thiophene-2-carbonyl, nicotinoyl, and 2-cyclopentylacetyl; and
R² and R³ together form CH₂.

9. The compound according to claim 1, wherein X is CH₂.

10. The compound according to claim 1, wherein R⁴ is selected from: H, fluoro, and chloro.

11. The compound according to claim 1, wherein R⁵ is selected from: H and fluoro.

12. The compound according to claim 1, wherein R⁶ is selected from: H and chloro.

13. The compound according to claim 1, wherein R⁷ is selected from: bromo and chloro.

14. The compound according to claim 1, selected from compounds of Formula (Ic) and pharmaceutically acceptable salts, solvates, and hydrates thereof: 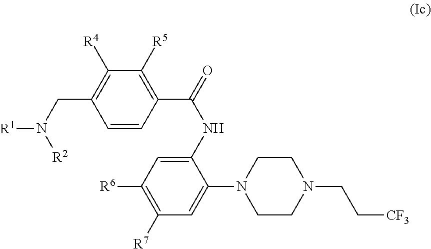
wherein:
R¹ is selected from: H, C₁-C₆ alkyl, C₁-C₆-alkyl-O—C₁-C₆-alkyl, C₃-C₇ cycloalkyl, C₁-C₁₃ cycloalkylalkyl, heteroaryl, heteroaryl-C₁-C₆-alkyl, heterocyclyl, and heterocyclyl-C₁-C₆-alkyl, each optionally substituted with one or more substituents selected from: C₁-C₆ alkoxycarbonylamino, amino-C₁-C₆-alkoxy, C₁-C₆ alkoxycarbonyl, C₁-C₆ alkyl, C₁-C₆ alkylcarboxamide, C₁-C₆ alkylsulfinyl, amino, carboxamide, carboxyl, cyano, C₂-C₆ dialkylamino, hydroxyl, hydroxy-C₁-C₆-alkyl, imino, oxo, phenyl, and phosphonooxy;
R² is selected from: H and C₁-C₆ alkyl, wherein said C₁-C₆ alkyl is optionally substituted with one or more substituents selected from: hydroxyl and cyano; and
R⁴, R⁵, R⁶, and R⁷ are each selected independently from: H and halogen.

15. The compound according to claim 1, selected from compounds of Formula (Ic) and pharmaceutically acceptable salts, solvates, and hydrates thereof: 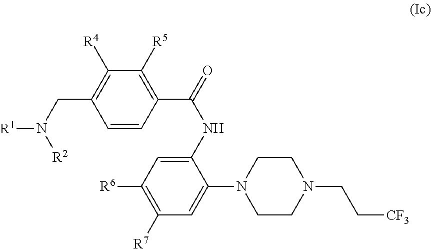
wherein:
R¹ is selected from: H, ethyl, 2-hydroxyethyl, 3-(1H-imidazol-1-yl)propyl, 4-methylpyridin-3-yl, methyl, 2-cyanoethyl, 2-amino-2-oxoethylamino, (1-methylpiperidin-4-yl)methyl, cyanomethyl, 1-amino-1-oxopropan-2-yl, 1,1-dioxo-tetrahydrothiophen-3-yl, 1-hydroxy-4-methylpentan-2-yl, 2-(1H-imidazol-5-yl)ethyl, (1-methyl-1H-imidazol-5-yl)methyl, 2-carbamoylcyclohexyl, 3-hydroxy-1-methoxy-1-oxopropan-2-yl, 1,3-dihydroxypropan-2-yl, 1-amino-3-hydroxy-1-oxopropan-2-yl, 2-hydroxycyclohexyl, 2-oxoazepan-3-yl, 2-(2-oxoimidazolidin-1-yl)ethyl, pyrrolidin-2-ylmethyl, pyrrolidin-3-yl, piperidin-3-yl, piperidin-4-yl, 2-hydroxypropyl, 2-hydroxypyridin-3-yl, 2-(4-methylpiperazin-1-yl)ethyl, 1-hydroxypropan-2-yl, 1,3-dihydroxy-2-(hydroxymethyl)propan-2-yl, 2-acetamidoethyl, 1-hydroxybutan-2-yl, 2-(1-methylpyrrolidin-2-yl)ethyl, 2-(dimethylamino)ethyl, 2-morpholinoethyl, 1-ethyl-2-oxoazepan-3-yl, 3-(dimethylamino)tetrahydrothiophen-3-yl)methyl, 2-(diethylamino)ethyl, 1-hydroxy-3-methylpentan-2-yl, 5-aminopentyl, 3-amino-1-imino-3-oxopropyl, (1-hydroxycyclohexyl)methyl, 2-(hydroxymethyl)pyrrolidin-1-yl)ethyl, 2-methyl-2-(piperidin-1-yl)propyl, benzyl, 2-(methylsulfinyl)ethyl, 2-(azepan-1-yl)ethyl, 3-hydroxybutyl, 1-amino-3-methyl-1-oxobutan-2-yl, 2-(2-(2-aminoethoxy)ethoxy)ethyl, 2-(hydroxymethyl)pyrrolidin-1-yl, 1,3-dihydroxybutan-2-yl, 2-morpholino-2-oxoethyl, 2-(dimethylamino)-2-(pyridin-3-yl)ethyl, 2-(pyrrolidin-1-yl)ethyl, 3-amino-1-methoxy-1-oxopropan-2-yl, 4-amino-1-methoxy-1-oxobutan-2-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, 2-hydroxycyclopentyl, piperidine-4-carbonyl, 2-aminocyclohexanecarbonyl, morpholine-2-carbonyl, 3-aminopropanoyl, 2-aminoacetyl, 4-hydroxypyrrolidine-2-carbonyl, 2-aminopropanoyl, 2-amino-3-hydroxypropanoyl, 2-hydroxyacetyl, thiomorpholine-3-carbonyl, pyrrolidine-2-carbonyl, 2-(morpholin-4-yl)acetyl, 2-(1H-tetrazol-5-yl)acetyl, 2-(dimethylamino)acetyl, 3-oxo-2,3-dihydroisoxazole-5-carbonyl, 6-oxo-1,6-dihydropyridazine-3-carbonyl, 2,4-dihydroxypyrimidine-5-carbonyl, 5-oxo-4,5-dihydro-1H-1,2,4-triazole-3-carbonyl, 4-aminotetrahydro-2H-thiopyran-4-carbonyl, 2-(3-amino-2-oxopyrrolidin-1-yl)acetyl, 6-hydroxynicotinoyl, 2-hydroxynicotinoyl, 2,6-dihydroxyisonicotinoyl, 2,6-dioxo-1,2,3,6-tetrahydropyrimidine-4-carbonyl, 5-hydroxy-1-methyl-1H-pyrazole-3-carbonyl, 3-(3-hydroxyisoxazol-4-yl)propanoyl, 3-carboxypropanoyl, 5-hydroxypyrazine-2-carbonyl, 6-hydroxypicolinoyl, 4-methylmorpholine-2-carbonyl, 4-ethylmorpholine-2-carbonyl, 4-(2-hydroxyethyl)morpholine-2-carbonyl, 4-(3,3-dimethylbutyl)morpholine-2-carbonyl, 4-(2-hydroxyethyl)morpholine-3-carbonyl, 4-ethylmorpholine-3-carbonyl, 4-(2-hydroxyethyl)thiomorpholine-3-carbonyl, 4-ethylthiomorpholine-3-carbonyl, 3-hydroxypropanoyl, 4-hydroxycyclohexanecarbonyl, 3-hydroxypentanoyl, 2-hydroxy-2-methylpropanoyl, 1-hydroxycyclopropanecarbonyl, 3-hydroxybutanoyl, 3-hydroxy-2,2-dimethylpropanoyl, 4-hydroxybutanoyl, 2-ethyl-2-hydroxybutanoyl, 2-hydroxycyclohexanecarbonyl, 2-cyclohexyl-2-hydroxyacetyl, 3-hydroxy-3-methylbutanoyl, 2-hydroxy-4-methylpentanoyl, 1-(tert-butoxycarbonyl)-4-hydroxypyrrolidine-2-carbonyl, 4-(tert-butoxycarbonyl)thiomorpholine-3-carbonyl, 2-(1-(tert-butoxycarbonyl)piperidin-2-yl)acetyl, 3-hydroxy-2-(hydroxymethyl)-2-methylpropanoyl, 2-(piperidin-2-yl)acetyl, 4-(hydroxymethyl)cyclohexanecarbonyl, 3-(dimethylamino)propanoyl, 2-(pyrrolidin-3-yl)acetyl, 3-(piperidin-1-yl)propanoyl, 4-aminocyclohexanecarbonyl, pyrrolidine-3-carbonyl, 3-(diethylamino)propanoyl, 2-(4-aminocyclohexyl)acetyl, 3-morpholinopropanoyl, 1-methylpiperidine-4-carbonyl, 3-aminocyclohexanecarbonyl, 2-amino-4-carboxybutanoyl, 4-amino-4-carboxybutanoyl, 3-aminocyclopentanecarbonyl, 1-methylpiperidine-3-carbonyl, 2-(piperidin-3-yl)acetyl, azetidine-3-carbonyl, 2-(4-(hydroxymethyl)piperidin-1-yl)acetyl, 2-(3-hydroxypiperidin-1-yl)acetyl, 2-(piperazin-1-yl)acetyl, 2-(3-aminopyrrolidin-1-yl)acetyl, 2-(2-(hydroxymethyl)morpholino)acetyl, 2-(4-propylpiperazin-1-yl)acetyl, 2-(5-oxo-1,4-diazepan-1-yl)acetyl, 2-(4-carbamoylpiperidin-1-yl)acetyl, 2-(2-carbamoylpyrrolidin-1-yl)acetyl, 2-(4-(dimethylamino)piperidin-1-yl)acetyl, 2-(3-(dimethylamino)pyrrolidin-1-yl)acetyl, 2-(4-hydroxypiperidin-1-yl)acetyl, 2-(2,5-diazabicyclo[2.2.1]heptan-2-yl)acetyl, 2-(hexahydropyrrolo[1,2-a]pyrazin-2(1H)-yl)acetyl, 2-(3-(hydroxymethyl)piperidin-1-yl)acetyl, 2-(3-methylpiperazin-1-yl)acetyl, 2-(4-methylpiperidin-1-yl)acetyl, 2-(3-oxopiperazin-1-yl)acetyl, 2-(4-carbamoylpiperazin-1-yl)acetyl, 2-(3-methylpiperidin-1-yl)acetyl, 2-(4-methylpiperazin-1-yl)acetyl, 2-(4-ethylpiperazin-1-yl)acetyl, 2-(2-(2-hydroxyethyl)piperidin-1-yl)acetyl, 2-(3-hydroxypyrrolidin-1-yl)acetyl, 2-(2-(hydroxymethyl)pyrrolidin-1-yl)acetyl, 2-(3-carbamoylpiperidin-1-yl)acetyl, 4-(phosphonooxy)cyclohexanecarbonyl, 2-(phosphonooxy)acetyl, 3-(tert-butoxycarbonylamino)pyrrolidine-1-carbonyl, 2-amino-4-methylpentanoyl, 2-amino-3-cyanopropanoyl, 4-amino-1,1-dioxotetrahydro-2H-thiopyran-4-carbonyl, 2,4-diamino-4-oxobutanoyl, 3-amino-2-hydroxypropanoyl, 2-hydroxypropanoyl, 5-(hydroxymethyl)-1H-1,2,3-triazole-4-carbonyl, piperazine-1-carbonyl, 4-ethylpiperazine-1-carbonyl, 1,1-dioxotetrahydro-2H-thiopyran-4-carbonyl, 3-hydroxypyrrolidine-1-carbonyl, 4-(2-hydroxyethyl)piperazine-1-carbonyl, 4-(hydroxymethyl)piperidine-1-carbonyl, 3-aminopiperidine-1-carbonyl, 3-hydroxyazetidine-1-carbonyl, 3-aminopyrrolidine-1-carbonyl, 2-carbamoylpyrrolidine-1-carbonyl, 4-(dimethylamino)piperidine-1-carbonyl, 4-carbamoylpiperazine-1-carbonyl, 3-oxopiperazine-1-carbonyl, 2-(hydroxymethyl)pyrrolidine-1-carbonyl, 2-(2-hydroxyethyl)piperidine-1-carbonyl, 2-(hydroxymethyl)morpholine-4-carbonyl, 3-carboxyazetidine-1-carbonyl, 4-(3-hydroxypropyl)piperidine-1-carbonyl, 3-hydroxypiperidine-1-carbonyl, 4-cyanopiperidine-1-carbonyl, 2-(hydroxymethyl)piperidine-1-carbonyl, 4-hydroxypiperidine-1-carbonyl, 2-oxopyrrolidine-1-carbonyl, 3-(hydroxymethyl)piperidine-1-carbonyl, 3-(hydroxymethyl)pyrrolidine-1-carbonyl, 3-(phosphonooxy)pyrrolidine-1-carbonyl, 1-(tert-butoxycarbonyl)piperidine-4-carbonyl, 2-(tert-butoxycarbonylamino)-cyclohexanecarbonyl, 1-(tert-butoxycarbonyl)piperidine-3-carbonyl, 3-(tert-butoxycarbonylamino)piperidine-1-carbonyl, 4-(tert-butoxycarbonyl)morpholine-2-carbonyl, 3-(tert-butoxycarbonylamino)propanoyl, 2-(tert-butoxycarbonylamino)acetyl, 3-(tert-butoxycarbonylamino)-2-hydroxypropanoyl, 2-(tert-butoxycarbonylamino)propanoyl, 2-(tert-butoxycarbonylamino)-3-hydroxypropanoyl, 1-(tert-butoxycarbonyl)pyrrolidine-2-carbonyl, 4-(tert-butoxycarbonylamino)tetrahydro-2H-thiopyran-4-carbonyl, 4-tert-butoxy-4-oxobutanoyl, 2-(3-(tert-butoxycarbonylamino)-2-oxopyrrolidin-1-yl)acetyl, 4-amino-2-(tert-butoxycarbonylamino)-4-oxobutanoyl, 2-(1-(tert-butoxycarbonyl)pyrrolidin-3-yl)acetyl, 4-(tert-butoxycarbonylamino)cyclohexanecarbonyl, 1-(tert-butoxycarbonyl)pyrrolidine-3-carbonyl, 2-(4-(tert-butoxycarbonylamino)cyclohexyl)acetyl, 3-(tert-butoxycarbonylamino)cyclohexanecarbonyl, 2-(tert-butoxycarbonylamino)-4-carboxybutanoyl, 4-(tert-butoxycarbonylamino)-4-carboxybutanoyl, 3-(tert-butoxycarbonylamino)cyclopentanecarbonyl, 2-(1-(tert-butoxycarbonyl)piperidin-3-yl)acetyl, 1-(tert-butoxycarbonyl)azetidine-3-carbonyl, 2-(3-(tert-butoxycarbonylamino)pyrrolidin-1-yl, 2-(4-(tert-butoxycarbonyl)-3-methylpiperazin-1-yl)acetyl, 2-(tert-butoxycarbonylamino)-4-methylpentanoyl, 2-(tert-butoxycarbonylamino)-3-cyanopropanoyl, and 4-(tert-butoxycarbonylamino)-1,1-dioxotetrahydro-2H-thiopyran-4-carbonyl;
R² is selected from: H, ethyl, methyl, isopropyl, 2-hydroxyethyl, 2-cyanoethyl, and tert-butyl;
R⁴ is selected from: H, fluoro, and chloro;
R⁵ is selected from: H and fluoro;
R⁶ is selected from: H, fluoro, and chloro; and
R⁷ is selected from: bromo and chloro.

16. The compound according to claim 1, selected from compounds of Formula (Ie) and pharmaceutically acceptable salts, solvates, and hydrates thereof: 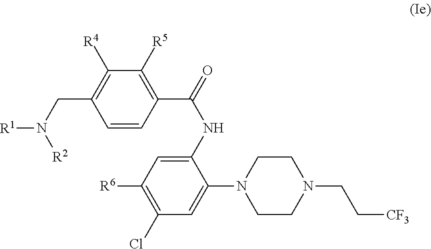
wherein:
R¹ and R² together with the nitrogen atom to which they are both bonded form a group selected from: 1,1-dioxo-thiomorpholin-4-yl, 3-hydroxypyrrolidin-1-yl, 4-(2-hydroxyethyl)piperazin-1-yl, hexahydropyrrolo[1,2-a]pyrazin-2(1H)-yl, 4-ethylpiperazin-1-yl, piperidin-1-yl, 1H-imidazol-1-yl, morpholino, 4-methylpiperazin-1-yl, pyrrolidin-1-yl, 1H-1,2,4-triazol-1-yl, 1H-pyrazol-1-yl, 1H-pyrrol-1-yl, 2H-tetrazol-5-yl, piperazin-1-yl, 4-(dimethylamino)piperidin-1-yl, 4-(hydroxymethyl)piperidin-1-yl, 2-carbamoylpyrrolidin-1-yl, 2-(2-hydroxyethyl)piperidin-1-yl, 4-carbamoylpiperazin-1-yl, 3-oxopiperazin-1-yl, 4-(2-cyclohexylethyl)piperazin-1-yl, 2,7-diazaspiro[4.4]nonan-2-yl, 3-(methylsulfonyl)pyrrolidin-1-yl, 6,7-dihydro-1H-imidazo[4,5-c]pyridin-5(4H)-yl, 2-(hydroxymethyl)piperidin-1-yl, 3-aminopyrrolidin-1-yl, 2-methylpiperazin-1-yl, 3-aminopiperidin-1-yl, 4-aminopiperidin-1-yl, 2-carbamoylpiperidin-1-yl, 5,6-dihydropyrimidin-1(4H)-yl, 4-hydroxy-2-(methoxycarbonyl)pyrrolidin-1-yl, 4-hydroxypiperidin-1-yl, 4-(2-(pyridin-2-yl)ethyl)piperazin-1-yl, 3-hydroxypiperidin-1-yl, 3-(diethylcarbamoyl)piperidin-1-yl, 2,3,4,6,7,8-hexahydro-1H-pyrimido[1,2-a]pyrimidin-1-yl, 4-cyclopentylpiperazin-1-yl, 1,4-oxazepan-4-yl, 2-(pyrrolidin-1-ylmethyl)pyrrolidin-1-yl, 4-morpholinopiperidin-1-yl, 4-(cyclohexylmethyl)piperazin-1-yl, 4-oxopiperidin-1-yl, 4-acetylpiperazin-1-yl, 1,4′-bipiperidin-1′-yl, 4-(ethoxycarbonyl)piperidin-1-yl, 2-(hydroxymethyl)morpholino, 2-(hydroxymethyl)pyrrolidin-1-yl, 3-hydroxyazetidin-1-yl, 4-hydroxy-1,4′-bipiperidin-1′-yl, 3-(hydroxymethyl)piperidin-1-yl, 2,5-diazabicyclo[2.2.1]heptan-2-yl, 5-oxo-1,4-diazepan-1-yl, 4-(2-hydroxyethyl)piperidin-1-yl, 3-(carboxymethyl)pyrrolidin-1-yl, 2,7-diazaspiro[3.5]nonan-2-yl, 4-(tert-butoxycarbonyl)-2-(carboxymethyl)piperazin-1-yl, 4-(tert-butoxycarbonyl)-2-carboxypiperazin-1-yl, 4-carboxypiperidin-1-yl, 2-(carboxymethyl)morpholino, 2-(carboxymethyl)piperazin-1-yl, 2-carboxypiperazin-1-yl, 4-(carboxymethyl)piperazin-1-yl, 2-carboxy-5,6-dihydroimidazo[1,2-a]pyrazin-7(8H)-yl, 2-carbamoylpiperazin-1-yl, 2-(methylcarbamoyl)piperazin-1-yl, 2-(2-hydroxyethylcarbamoyl)piperazin-1-yl, 2-(1-hydroxypropan-2-ylcarbamoyl)piperazin-1-yl, 3-carbamoylpiperidin-1-yl, 4-carbamoylpiperidin-1-yl, 3-(hydroxymethyl)pyrrolidin-1-yl, 2-oxopyrrolidin-1-yl, 2,5-dioxoimidazolidin-1-yl, 2,6-dioxotetrahydropyrimidin-1(2H)-yl, 3-methyl-2,5-dioxoimidazolidin-1-yl, and 4-isobutyl-2,5-dioxoimidazolidin-1-yl;
R⁴ is selected from: H and fluoro;
R⁵ is selected from: H and fluoro; and
R⁶ is selected from: H and chloro.

17. The compound according to claim 1, selected from compounds of Formula (Ig) and pharmaceutically acceptable salts, solvates, and hydrates thereof: 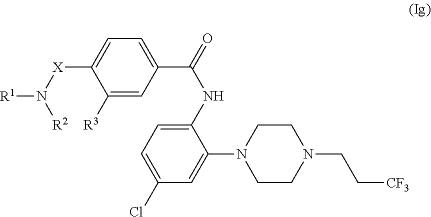
wherein:
X is CH₂ or CH₂CH₂;
R¹ is selected from: H, C₁-C₆ alkyl, C₁-C₆-alkyl-O—C₁-C₆-alkyl, and heterocyclyl-C₁-C₆-alkyl, each optionally substituted with one or more substituents selected from: C₁-C₆ alkoxycarbonyl, C₁-C₆ alkylsulfonyl, carboxamide, cyano, C₂-C₆ dialkylamino, and hydroxyl; and
R² and R³ together form CH₂.

18. The compound according to claim 1, selected from compounds of Formula (Ig) and pharmaceutically acceptable salts, solvates, and hydrates thereof: 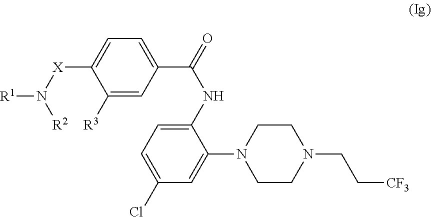
wherein:
X is CH₂ or CH₂CH₂;
R¹ is selected from: H, methyl, butyl, 3-hydroxypropyl, 3,3-dimethylbutyl, (tetrahydro-2H-pyran-4-yl)methyl, 2-methoxyethyl, 3-amino-3-oxopropyl, 2-hydroxyethyl, 2-ethoxy-2-oxoethyl, 2-amino-2-oxoethyl, cyanomethyl, 2-ethoxyethyl, 2-(diethylamino)ethyl, 2-(methylsulfonyl)ethyl, butyr-1-yl, 2-ethylbutanoyl, thiophene-2-carbonyl, nicotinoyl, and 2-cyclopentylacetyl;
R² and R³ together form CH₂.

19. The compound according to claim 1, selected from compounds of Formula (II) and pharmaceutically acceptable salts, solvates, and hydrates thereof: 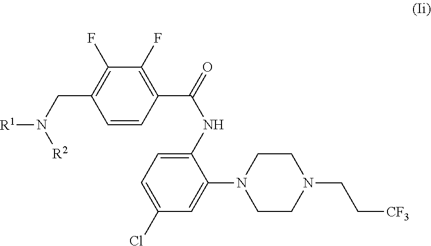
wherein:
R¹ is selected from: H, 2-hydroxyethyl, 2-cyanoethyl, 1,1-dioxo-tetrahydrothiophen-3-yl, 2-carbamoylcyclohexyl, 1-amino-3-hydroxy-1-oxopropan-2-yl, 2-hydroxycyclohexyl, piperidin-3-yl, piperidin-4-yl, 1-carboxy-2-hydroxyethyl, (2H-tetrazol-5-yl)methyl, 3-oxo-2,3-dihydroisoxazol-5-yl)methyl, carboxymethyl, 3-carboxypropyl, 2-carboxyethyl, 3-amino-1-carboxy-3-oxopropyl, 1-carboxy-3-methylbutyl, 1,3-dicarboxypropyl, 2-carboxypropan-2-yl, 4-carboxy-1-methoxy-1-oxobutan-2-yl, 3-carboxy-1-methoxy-1-oxopropan-2-yl, 3-(tert-butoxycarbonylamino)-1-carboxypropyl, 2-(tert-butoxycarbonylamino)-1-carboxyethyl, 3-amino-1-carboxypropyl, 2-amino-1-carboxyethyl, 5-carboxypentyl, 1-amino-1-oxo-3-(phosphonooxy)propan-2-yl, 2-carbamoylcyclopentyl, and 2-hydroxycyclopentyl; and
R² is selected from: H, ethyl, methyl, and 2-hydroxyethyl.

20. The compound according to claim 1, selected from compounds of Formula (Ik) and pharmaceutically acceptable salts, solvates, and hydrates thereof: 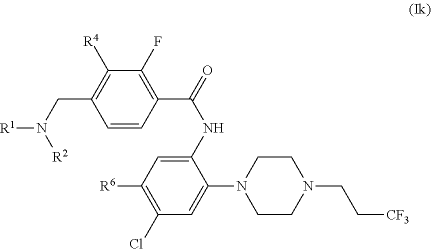
wherein:
R¹ is selected from: C₁-C₆ alkyl, C₁-C₁₃ cycloalkylalkyl, heteroaryl-C₁-C₆-alkyl, and heterocyclyl-C₁-C₆-alkyl, each optionally substituted with one or more substituents selected from: amino, carboxamide, hydroxyl, hydroxy-C₁-C₆-alkyl, oxo, and phosphonooxy; and
R² is H; or
R¹ and R² together with the nitrogen atom to which they are both bonded form a heterocyclyl group optionally substituted with one or more oxo substituents; and
R⁴ and R⁶ are each selected independently from: H and halogen.

21. The compound according to claim 1, selected from compounds of Formula (Ik) and pharmaceutically acceptable salts, solvates, and hydrates thereof: 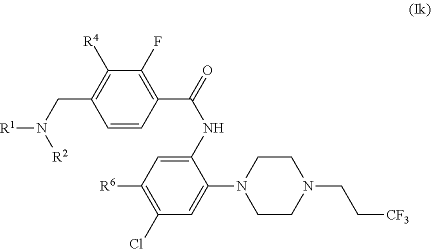
wherein:
R¹ is selected from: ethyl, propan-1-yl, propan-2-yl, butan-1-yl, isobutyl, morpholin-2-ylmethyl, 2-(morpholin-4-yl)ethyl, (4,5-dihydro-1H-1,2,4-triazol-3-yl)methyl, pyridin-3-ylmethyl, pyrazin-2-ylmethyl, cyclohexylmethyl, 4-methylpentyl, pyrrolidin-1-ylmethyl, (1,1-dioxotetrahydro-2H-thiopyran-4-yl)methyl, piperidin-1-ylmethyl, piperazin-1-ylmethyl, azetidin-1-ylmethyl, and (morpholin-4-yl)methyl; each optionally substituted with one or more substituents selected from: amino, carboxamide, hydroxyl, hydroxymethyl, oxo, and phosphonooxy; and
R² is H; or
R¹ and R² together with the nitrogen atom to which they are both bonded form a piperazinyl group optionally substituted with one or more oxo substituents; and
R⁴ and R⁶ are each selected independently from: H, fluoro, and chloro.

22. The compound according to claim 1, selected from compounds of Formula (Ik) and pharmaceutically acceptable salts, solvates, and hydrates thereof: 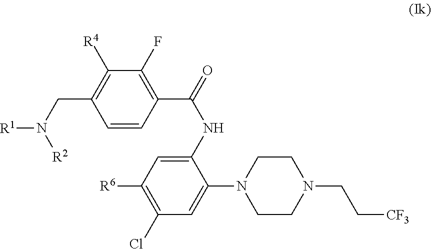
wherein:
R¹ is selected from: 1-amino-3-hydroxy-1-oxopropan-2-yl, 1-amino-1-oxo-3-(phosphonooxy)propan-2-yl, 2-hydroxyacetyl, morpholine-2-carbonyl, 2-(morpholin-4-yl)acetyl, 5-oxo-4,5-dihydro-1H-1,2,4-triazole-3-carbonyl, 2-hydroxynicotinoyl, 5-hydroxypyrazine-2-carbonyl, 4-hydroxycyclohexanecarbonyl, 2-hydroxy-2-methylpropanoyl, 1-hydroxycyclopropanecarbonyl, 3-hydroxybutanoyl, 2-hydroxy-4-methylpentanoyl, 3-hydroxy-2-(hydroxymethyl)-2-methylpropanoyl, 4-(hydroxymethyl)cyclohexanecarbonyl, 4-(phosphonooxy)cyclohexanecarbonyl, 2-(phosphonooxy)acetyl, 4-amino-1,1-dioxotetrahydro-2H-thiopyran-4-carbonyl, 2-hydroxypropanoyl, 3-hydroxypyrrolidine-1-carbonyl, 4-(hydroxymethyl)piperidine-1-carbonyl, 3-hydroxyazetidine-1-carbonyl, 2-carbamoylpyrrolidine-1-carbonyl, 3-oxopiperazine-1-carbonyl, 2-(hydroxymethyl)pyrrolidine-1-carbonyl, 2-(hydroxymethyl)morpholine-4-carbonyl, 3-hydroxypiperidine-1-carbonyl, 4-hydroxypiperidine-1-carbonyl, 3-(phosphonooxy)pyrrolidine-1-carbonyl, and 3-(hydroxymethyl)pyrrolidine-1-carbonyl; and
R² is H; or
R¹ and R² together with the nitrogen atom to which they are both bonded form a 3-oxopiperazin-1-yl group;
R⁴ is selected from: H and fluoro; and
R⁶ is selected from: H and chloro.

23. The compound according to claim 1, selected from the following compounds and pharmaceutically acceptable salts, solvates, and hydrates thereof:
N-(4-chloro-2-(4-(3,3,3-trifluoropropyl)piperazin-1-yl)phenyl)-2,3-difluoro-4-((2-hydroxyacetamido)methyl)benzamide (Compound 270);
(S)—N-(4-(4-chloro-2-(4-(3,3,3-trifluoropropyl)piperazin-1-yl)phenylcarbamoyl)-2,3-difluorobenzyl)morpholine-2-carboxamide (Compound 271);
(R)—N-(4-(4-chloro-2-(4-(3,3,3-trifluoropropyl)piperazin-1-yl)phenylcarbamoyl)-2,3-difluorobenzyl)morpholine-2-carboxamide (Compound 272);
N-(4-chloro-2-(4-(3,3,3-trifluoropropyl)piperazin-1-yl)phenyl)-2,3-difluoro-4-((2-morpholinoacetamido)methyl)benzamide (Compound 277);
N-(4-(4-chloro-2-(4-(3,3,3-trifluoropropyl)piperazin-1-yl)phenylcarbamoyl)-2,3-difluorobenzyl)-5-oxo-4,5-dihydro-1H-1,2,4-triazole-3-carboxamide (Compound 283);
N-(4-(4-chloro-2-(4-(3,3,3-trifluoropropyl)piperazin-1-yl)phenylcarbamoyl)-2,3-difluorobenzyl)-2-hydroxynicotinamide (Compound 288);
N-(4-(4-chloro-2-(4-(3,3,3-trifluoropropyl)piperazin-1-yl)phenylcarbamoyl)-2,3-difluorobenzyl)-5-hydroxypyrazine-2-carboxamide (Compound 294);
N-(4-chloro-2-(4-(3,3,3-trifluoropropyl)piperazin-1-yl)phenyl)-2,3-difluoro-4-((2-hydroxy-2-methylpropanamido)methyl)benzamide (Compound 310);
N-(4-chloro-2-(4-(3,3,3-trifluoropropyl)piperazin-1-yl)phenyl)-2,3-difluoro-4-((1-hydroxycyclopropanecarboxamido)methyl)benzamide (Compound 311);
N-(4-chloro-2-(4-(3,3,3-trifluoropropyl)piperazin-1-yl)phenyl)-2,3-difluoro-4-(((1r,4r)-4-hydroxycyclohexanecarboxamido)methyl)benzamide (Compound 312);
(R)—N-(4-chloro-2-(4-(3,3,3-trifluoropropyl)piperazin-1-yl)phenyl)-2,3-difluoro-4-((3-hydroxybutanamido)methyl)benzamide (Compound 313);
(S)—N-(4-chloro-2-(4-(3,3,3-trifluoropropyl)piperazin-1-yl)phenyl)-2,3-difluoro-4-((3-hydroxybutanamido)methyl)benzamide (Compound 315);
(R)—N-(4-chloro-2-(4-(3,3,3-trifluoropropyl)piperazin-1-yl)phenyl)-2,3-difluoro-4-((2-hydroxy-4-methylpentanamido)methyl)benzamide (Compound 322);
N-(4-chloro-2-(4-(3,3,3-trifluoropropyl)piperazin-1-yl)phenyl)-2,3-difluoro-4-((3-hydroxy-2-(hydroxymethyl)-2-methylpropanamido)methyl)benzamide (Compound 326);
N-(4-chloro-2-(4-(3,3,3-trifluoropropyl)piperazin-1-yl)phenyl)-2,3-difluoro-4-(((1r,4r)-4-(hydroxymethyl)cyclohexanecarboxamido)methyl)benzamide (Compound 329);
N-(4-chloro-2-(4-(3,3,3-trifluoropropyl)piperazin-1-yl)phenyl)-2,3-difluoro-4-(((1s,4s)-4-hydroxycyclohexanecarboxamido)methyl)benzamide (Compound 330);
(1r,4r)-4-(4-(4-chloro-2-(4-(3,3,3-trifluoropropyl)piperazin-1-yl)phenylcarbamoyl)-2,3-difluorobenzylcarbamoyl)cyclohexyl dihydrogen phosphate (Compound 381);
2-(4-(4-chloro-2-(4-(3,3,3-trifluoropropyl)piperazin-1-yl)phenylcarbamoyl)-2,3-difluorobenzylamino)-2-oxoethyl dihydrogen phosphate (Compound 382);
N-(4-chloro-2-(4-(3,3,3-trifluoropropyl)piperazin-1-yl)phenyl)-2,3-difluoro-4-((4-(hydroxymethyl)cyclohexanecarboxamido)methyl)benzamide (Compound 385);
4-amino-N-(4-(4-chloro-2-(4-(3,3,3-trifluoropropyl)piperazin-1-yl)phenylcarbamoyl)-2,3-difluorobenzyl)-1,1-dioxotetrahydro-2H-thiopyran-4-carboxamide (Compound 388);
N-(4-chloro-2-(4-(3,3,3-trifluoropropyl)piperazin-1-yl)phenyl)-2,3-difluoro-4-((2-hydroxypropanamido)methyl)benzamide (Compound 391);
(1s,4s)-4-(4-(4-chloro-2-(4-(3,3,3-trifluoropropyl)piperazin-1-yl)phenylcarbamoyl)-2,3-difluorobenzylcarbamoyl)cyclohexyl dihydrogen phosphate (Compound 392);
(S)—N-(4-(4-Chloro-2-(4-(3,3,3-trifluoropropyl)piperazin-1-yl)phenylcarbamoyl)-2,3-difluorobenzyl)-3-hydroxypyrrolidine-1-carboxamide (Compound 406);
N-(4-(4-Chloro-2-(4-(3,3,3-trifluoropropyl)piperazin-1-yl)phenylcarbamoyl)-2,3-difluorobenzyl)-4-(hydroxymethyl)piperidine-1-carboxamide (Compound 407);
(S)—N¹-(4-(4-Chloro-2-(4-(3,3,3-trifluoropropyl)piperazin-1-yl)phenylcarbamoyl)-2,3-difluorobenzyl)pyrrolidine-1,2-dicarboxamide (Compound 408);
N-(4-(4-Chloro-2-(4-(3,3,3-trifluoropropyl)piperazin-1-yl)phenylcarbamoyl)-2,3-difluorobenzyl)-3-oxopiperazine-1-carboxamide (Compound 412);
(R)—N-(4-(4-Chloro-2-(4-(3,3,3-trifluoropropyl)piperazin-1-yl)phenylcarbamoyl)-2,3-difluorobenzyl)-2-(hydroxymethyl)pyrrolidine-1-carboxamide (Compound 413);
N-(4-(4-Chloro-2-(4-(3,3,3-trifluoropropyl)piperazin-1-yl)phenylcarbamoyl)-2,3-difluorobenzyl)-3-hydroxyazetidine-1-carboxamide (Compound 415);
N-(4-(4-Chloro-2-(4-(3,3,3-trifluoropropyl)piperazin-1-yl)phenylcarbamoyl)-2,3-difluorobenzyl)-2-(hydroxymethyl)morpholine-4-carboxamide (Compound 416);
N-(4-(4-Chloro-2-(4-(3,3,3-trifluoropropyl)piperazin-1-yl)phenylcarbamoyl)-2,3-difluorobenzyl)-4-hydroxypiperidine-1-carboxamide (Compound 424);
(R)—N-(4-(4-Chloro-2-(4-(3,3,3-trifluoropropyl)piperazin-1-yl)phenylcarbamoyl)-2,3-difluorobenzyl)-3-hydroxypyrrolidine-1-carboxamide (Compound 425);
(R)—N-(4-(4-Chloro-2-(4-(3,3,3-trifluoropropyl)piperazin-1-yl)phenylcarbamoyl)-2,3-difluorobenzyl)-3-hydroxypiperidine-1-carboxamide (Compound 436);
(S)—N-(4-(4-Chloro-2-(4-(3,3,3-trifluoropropyl)piperazin-1-yl)phenylcarbamoyl)-2,3-difluorobenzyl)-3-hydroxypiperidine-1-carboxamide (Compound 437);
(S)—N-(4-(4-Chloro-2-(4-(3,3,3-trifluoropropyl)piperazin-1-yl)phenylcarbamoyl)-2,3-difluorobenzyl)-2-(hydroxymethyl)pyrrolidine-1-carboxamide (Compound 438);
(R)—N-(4-(4-Chloro-2-(4-(3,3,3-trifluoropropyl)piperazin-1-yl)phenylcarbamoyl)-2,3-difluorobenzyl)-3-(hydroxymethyl)pyrrolidine-1-carboxamide (Compound 439);
(S)-1-(4-(4-Chloro-2-(4-(3,3,3-trifluoropropyl)piperazin-1-yl)phenylcarbamoyl)-2,3-difluorobenzylcarbamoyl)pyrrolidin-3-yl dihydrogen phosphate (Compound 440); and
(R)-1-(4-(4-Chloro-2-(4-(3,3,3-trifluoropropyl)piperazin-1-yl)phenylcarbamoyl)-2,3-difluorobenzylcarbamoyl)pyrrolidin-3-yl dihydrogen phosphate (Compound 441).

24. The compound according to claim 1, selected from the following compounds and pharmaceutically acceptable salts, solvates, and hydrates thereof:
(S)-4-((1-Amino-3-hydroxy-1-oxopropan-2-ylamino)methyl)-N-(4-chloro-2-(4-(3,3,3-trifluoropropyl)piperazin-1-yl)phenyl)-2-fluorobenzamide (Compound 76);
(S)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170);
(S)-4-((1-Amino-3-hydroxy-1-oxopropan-2-ylamino)methyl)-N-(4,5-dichloro-2-(4-(3,3,3-trifluoropropyl)piperazin-1-yl)phenyl)-2,3-difluorobenzamide (Compound 185);
N-(4,5-Dichloro-2-(4-(3,3,3-trifluoropropyl)piperazin-1-yl)phenyl)-2,3-difluoro-4-((3-oxopiperazin-1-yl)methyl)benzamide (Compound 188);
(R)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 247);
(S)-3-Amino-2-(4-(4-chloro-2-(4-(3,3,3-trifluoropropyl)piperazin-1-yl)phenylcarbamoyl)-2,3-difluorobenzylamino)-3-oxopropyl dihydrogenphosphate (Compound 249); and
(R)-3-Amino-2-(4-(4-chloro-2-(4-(3,3,3-trifluoropropyl)piperazin-1-yl)phenylcarbamoyl)-2,3-difluorobenzylamino)-3-oxopropyl dihydrogenphosphate (Compound 250).

25. The compound according to claim 1, selected from the following compound and pharmaceutically acceptable salts, solvates, and hydrates thereof:
(S)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170).

26. The compound according to claim 1, selected from the following compound and pharmaceutically acceptable salts, solvates, and hydrates thereof:
(S)-4-((1-Amino-3-hydroxy-1-oxopropan-2-ylamino)methyl)-N-(4-chloro-2-(4-(3,3,3-trifluoropropyl)piperazin-1-yl)phenyl)-2-fluorobenzamide (Compound 76).

27. The compound according to claim 1, selected from the following compound and pharmaceutically acceptable salts, solvates, and hydrates thereof:
(R)-3-Amino-2-(4-(4-chloro-2-(4-(3,3,3-trifluoropropyl)piperazin-1-yl)phenylcarbamoyl)-2,3-difluorobenzylamino)-3-oxopropyl Dihydrogenphosphate (Compound 250).

28. The compound according to claim 1, selected from the following compound and pharmaceutically acceptable salts, solvates, and hydrates thereof:
(S)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170).

29. The compound according to claim 1, selected from the following compound and pharmaceutically acceptable salts, solvates, and hydrates thereof:
(S)-3-Amino-2-(4-(4-chloro-2-(4-(3,3,3-trifluoropropyl)piperazin-1-yl)phenylcarbamoyl)-2,3-difluorobenzylamino)-3-oxopropyl dihydrogenphosphate (Compound 249).

30. The compound according to claim 1, selected from the following compound and pharmaceutically acceptable salts, solvates, and hydrates thereof:
(S)—N-(4-(4-Chloro-2-(4-(3,3,3-trifluoropropyl)piperazin-1-yl)phenylcarbamoyl)-2,3-difluorobenzyl)morpholine-2-carboxamide (Compound 271).

31. The compound according to claim 1, selected from the following compound and pharmaceutically acceptable salts, solvates, and hydrates thereof:
(R)—N-(4-(4-Chloro-2-(4-(3,3,3-trifluoropropyl)piperazin-1-yl)phenylcarbamoyl)-2,3-difluorobenzyl)morpholine-2-carboxamide (Compound 272).

32. The compound according to claim 1, selected from the following compound and pharmaceutically acceptable salts, solvates, and hydrates thereof:
N-(4-Chloro-2-(4-(3,3,3-trifluoropropyl)piperazin-1-yl)phenyl)-2,3-difluoro-4-(((1r,4r)-4-hydroxycyclohexanecarboxamido)methyl)benzamide (Compound 312).

33. The compound according to claim 1, selected from the following compound and pharmaceutically acceptable salts, solvates, and hydrates thereof:
(1r,4r)-4-(4-(4-Chloro-2-(4-(3,3,3-trifluoropropyl)piperazin-1-yl)phenylcarbamoyl)-2,3-difluorobenzylcarbamoyl)cyclohexyl dihydrogen phosphate (Compound 381).

34. A crystalline form of the compound according to claim 25, selected from the following compound and pharmaceutically acceptable salts, solvates, and hydrates thereof:
(S)-4-((1-Amino-3-hydroxy-1-oxopropan-2-ylamino)methyl)-N-(4-chloro-2-(4-(3,3,3-trifluoropropyl)piperazin-1-yl)phenyl)-2,3-difluorobenzamide (Compound 170).

35. A composition comprising a compound according to claim 1.

36. A pharmaceutical product selected from: a pharmaceutical composition, a formulation, a unit dosage form, and a kit, each comprising a compound according to claim 1.

37. A pharmaceutical composition comprising a compound according to claim 1, and a pharmaceutically acceptable carrier.

38. A method for preparing a pharmaceutical composition comprising the step of admixing a compound according to claim 1, and a pharmaceutically acceptable carrier.

39. A method for the treatment of a Mas receptor-mediated disorder in an individual, comprising administering to said individual in need thereof, a therapeutically effective amount of a compound according to claim 1.

40. A method for the treatment of: a disorder alleviated by vasodilation in an individual, ischemia reperfusion injury during and/or following coronary bypass surgery, ischemia reperfusion myocardial injury during and/or following coronary bypass surgery, a disorder alleviated by inhibiting calcium signaling in cells in an individual, a disorder alleviated by correcting improper calcium handling by cells in an individual, arrhythmia in an individual, ischemia reperfusion-induced arrhythmia, reperfusion-induced myocardial injury in an individual, reperfusion-induced cardiomyocyte injury in an individual, reperfusion-induced cardiomyocyte cell death in an individual, or an inflammatory disorder in an individual, comprising administering to said individual in need thereof, a therapeutically effective amount of a compound according to claim 1.

41. A method for: reducing injury due to blood clot formation in an individual, reducing injury due to blood clot formation following angioplasty in an individual, providing neuroprotection in an individual, or providing renal protection in an individual, comprising administering to said individual in need thereof, a therapeutically effective amount of a compound according to claim 1.

42. (canceled)

43. (canceled)

44. (canceled)

45. (canceled)

46. (canceled)

47. (canceled)

48. (canceled)

49. A method according to claim 39; wherein said Mas receptor-mediated disorder is selected from: coronary heart disease, atherosclerosis, ischemia, reperfusion injury, reperfusion injury following cardioplegia, reperfusion injury following angioplasty, angina pectoris, myocardial infarction, no-reflow phenomenon, hypertension, pulmonary hypertension, anxiety, transient ischemic attack, erectile dysfunction, ischemic colitis, mesenteric ischemia, acute limb ischemia, skin discoloration caused by reduced blood flow to the skin, renal artery stenosis, renovascular hypertension, renal failure, chronic kidney disease, and diabetic nephropathy.

50. A method according to claim 39; wherein said Mas receptor-mediated disorder is selected from: stroke, brain attack, neuroprotection, brain ischemia (thrombotic, embolic and hypoperfusion), focal or multifocal brain ischemia, global brain ischemia, ischemic brain injury, acute ischemic brain damage, acute ischemic brain injury, brain infarction, brain reperfusion injury, brain hypoxia, cerebral reperfusion injury, neuronal reperfusion injury, ischemic neurological disorders, ischemic brain damage, cerebral hypoxia, cerebral ischemia, cerebral ischemic injury, hypoxic-ischemic brain injury, anoxic brain injury, anoxic brain damage, anoxic encepalopathy, subcortical ischemic depression, moyamoya disease, and cardiorespiratory arrest.

51. A method according to claim 39; wherein said Mas receptor-mediated disorder is selected from: nephropathy, nephrotic syndrome, obstruction nephropathy, obstructive nephropathy, diabetic nephropathy, renal hypertension, renovascular hypertension, renal ischemia, renal ischemic injury, renal ischemia-reperfusion injury, renal reperfusion injury, acute renal injury, acute kidney injury, acute renal failure, acute kidney failure, acute tubular necrosis, contrast nephropathy, chronic kidney disease, chronic renal failure, chronic renal insufficiency, end stage renal disease, end stage renal failure, focal segmental glomerulosclerosis, glomerulonephritis, diabetes and diabetic kidney disease, diabetes insipidus, Fabry's disease, focal segmental glomerulosclerosis, focal sclerosis, focal glomerulosclerosis, Gitelman syndrome, glomerular diseases, high blood pressure and kidney disease, IgA nephropathy (Berger's disease), interstitial nephritis, lupus, malignant hypertension, microscopic polyangiitis (MPA), preeclampsia, polyarteritis, proteinuria, renal artery stenosis, renal infarction, reflux nephropathy, scleroderma renal crisis, tuberous sclerosis, and warfarin-related nephropathy.

52. A method according to claim 39; wherein said Mas receptor-mediated disorder is reperfusion-induced myocardial injury in an individual.

53. A method according to claim 40; wherein said inflammatory disorder is selected from: a TNFα mediated disorder, inflammatory bowel disease (IBD), inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, septic shock, endotoxic shock, atherosclerosis, ischemia-reperfusion injury, coronary heart disease, vasculitis, amyloidosis, multiple sclerosis, sepsis, chronic recurrent uveitis, hepatitis C virus infection, malaria, ulcerative colitis, cachexia, plasmocytoma, endometriosis, Behcet's disease, Wegenrer's granulomatosis, autoimmune diseases such as Crohn's disease, psoriasis or ankylosing spondylitis, immune deficiency, common variable immunodeficiency (CVID), chronic graft-versus-host disease, trauma and transplant rejection, adult respiratory distress syndrome, pulmonary fibrosis, recurrent ovarian cancer, lymphoproliferative disease, refractory multiple myeloma, myeloproliferative disorder, diabetes, juvenile diabetes, meningitis, skin delayed type hypersensitivity disorders, Alzheimer's disease, systemic lupus erythematosus and allergic asthma.
